SEQUENCE LISTING (109 sequences)

SEQ ID NO: 1 - protein, 85 aa, ARTIFICIAL SEQUENCE
  TARGET BINDING MOEITY
DEDLEELERLYRKAEEVAKEAKDASRRGDDERAKEQMERAMRLFDQVFELAQELQEKQTDGNRQKATHLDKAVKEAADELYQRVR

SEQ ID NO: 2 - protein, 75 aa, ARTIFICIAL SEQUENCE
  TARGET BINDING MOEITY
ELEEQVMHVLDQVSELAHELLHKLTGEELERAAYFNWWATEMMLELIKSDDEREIREIEEEARRILEHLEELARK

SEQ ID NO: 3 - protein, 56 aa, ARTIFICIAL SEQUENCE
  TARGET BINDING MOEITY
DKEWILQKIYEIMRLLDELGHAEASMRVSDLIYEFMKKGDERLLEEAERLLEEVER

SEQ ID NO: 4 - protein, 56 aa, ARTIFICIAL SEQUENCE
  TARGET BINDING MOEITY
DKEEILNKIYEIMRLLDELGNAEASMRVSDLILEFMKKGDERLLEEAERLLEEVER

SEQ ID NO: 5 - protein, 56 aa, ARTIFICIAL SEQUENCE
  TARGET BINDING MOEITY
SDDEDSVRYLLYMAELRYEQGNPEKAKKILEMAEFIAKRNNNEELERLVREVKKRL

SEQ ID NO: 6 - protein, 64 aa, ARTIFICIAL SEQUENCE
  TARGET BINDING MOEITY
NDDELHMLMTDLVYEALHFAKDEEIKKRVFQLFELADKAYKNNDRQKLEKVVEELKELLERLLS

SEQ ID NO: 7 - protein, 56 aa, ARTIFICIAL SEQUENCE
  TARGET BINDING MOEITY
QREKRLKQLEMLLEYAIERNDPYLMFDVAVEMLRLAEENNDERIIERAKRILEEYE

SEQ ID NO: 8 - protein, 65 aa, ARTIFICIAL SEQUENCE
  TARGET BINDING MOEITY
SLEELKEQVKELKKELSPEMRRLIEEALRFLEEGNPAMAMMVLSDLVYQLGDPRVIDLYMLVTKT

SEQ ID NO: 9 - protein, 56 aa, ARTIFICIAL SEQUENCE
  TARGET BINDING MOEITY
DREQRLVRFLVRLASKFNLSPEQILQLFEVLEELLERGVSEEEIRKQLEEVAKELG

SEQ ID NO: 10 - protein, 57 aa, ARTIFICIAL SEQUENCE
  TARGET BINDING MOEITY
DDDIRYLIYMAKLRLEQGNPEEAEKVLEMARFLAERLGMEELLKEVRELLRKIEELR

SEQ ID NO: 11 - protein, 65 aa, ARTIFICIAL SEQUENCE
  TARGET BINDING MOEITY
PIIELLREAKEKNDEFAISDALYLVNELLQRTGDPRLEEVLYLIWRALKEKDPRLLDRAIELFER

SEQ ID NO: 12 - protein, 4 aa, Artificial sequence
  Peptide unit
APAR

SEQ ID NO: 13 - protein, 4 aa, Artificial sequence
  peptide unit
RAPA

SEQ ID NO: 14 - protein, 4 aa, artificial sequence
  peptide unite
  misc_feature (1)..(1): aromatic amino acid
  misc_feature (2)..(2): any amino acid
  misc_feature (3)..(4): positively charged amino acid
XXXX

SEQ ID NO: 15 - protein, 4 aa, artificial sequence
  unit peptide
  misc_feature (1)..(2): positively charged amino acid
  misc_feature (3)..(3): any amino acid
  misc_feature (4)..(4): aromatic amino acid
XXXX

SEQ ID NO: 16 - protein, 7 aa, artificial sequence
  unit peptide
  misc_feature (1)..(1): any positively charged amino acid
  misc_feature (2)..(2): any aromatic amino acid
  misc_feature (3)..(3): any positively charged amino acid
  misc_feature (4)..(4): any amino acid
  misc_feature (5)..(5): any positively charged amino acid
  misc_feature (6)..(6): any aromatic amino acid
  misc_feature (7)..(7): any positively charged amino acid
XXXXXXX

SEQ ID NO: 17 - protein, 5 aa, artificial sequence
  unit peptide
  misc_feature (1)..(2): any positively charged amino acid
  misc_feature (3)..(4): any aromatic amino acid
  misc_feature (5)..(5): any positively charged amino acid
XXXXX

SEQ ID NO: 18 - protein, 4 aa, artificial sequence
  unit peptide
  misc_feature (1)..(3): any positively charged amino acid
  misc_feature (4)..(4): any aromatic amino acid
XXXX

SEQ ID NO: 19 - protein, 6 aa, artificial sequence
  peptide unit
HWRGWA

SEQ ID NO: 20 - protein, 4 aa, artificial sequence
  peptide unit
WGRR

SEQ ID NO: 21 - protein, 4 aa, artificial sequence
  peptide unit
RRGW

SEQ ID NO: 22 - protein, 8 aa, artificial sequence
  peptide unit
NKFRGKYK

SEQ ID NO: 23 - protein, 8 aa, artificial sequence
  peptide unit
NRFRGKYK

SEQ ID NO: 24 - protein, 7 aa, artificial sequence
  peptide unit
NARKFYK

SEQ ID NO: 25 - protein, 8 aa, artificial sequence
  peptide unit
NARKFYKG

SEQ ID NO: 26 - protein, 6 aa, artificial sequence
  peptide unit
HWRGWV

SEQ ID NO: 27 - protein, 7 aa, artificial sequence
  peptide unit
KHFRNKD

SEQ ID NO: 28 - protein, 7 aa, artificial sequence
  peptide unit
RHRFNKD

SEQ ID NO: 29 - protein, 4 aa, artificial sequence
  peptide unit
RTYK

SEQ ID NO: 30 - protein, 4 aa, artificial sequence
  peptide unit
ELVW

SEQ ID NO: 31 - protein, 5 aa, artificial sequence
  peptide unit
GELVW

SEQ ID NO: 32 - protein, 9 aa, artificial sequence
  peptide unit
AWHLGELVW

SEQ ID NO: 33 - protein, 9 aa, artificial sequence
  peptide unit
AWDLGELVW

SEQ ID NO: 34 - protein, 9 aa, artificial sequence
  peptide unit
  misc_feature (3)..(3): any amino acid residue comprising -COOH
AWXLGELVW

SEQ ID NO: 35 - protein, 13 aa, artificial sequence
  peptide unit
  disulfid (2)..(12)
DCAWHLGELVWCT

SEQ ID NO: 36 - protein, 13 aa, artificial sequence
  peptide unit
  disulfid (2)..(12)
  misc_feature (5)..(5): an amino acid residue comprising -COOH
  BINDING (5)..(5): binding to another moiety
DCAWXLGELVWCT

SEQ ID NO: 37 - protein, 13 aa, artificial sequence
  peptide unit
  disulfid (2)..(12)
DCAWDLGELVWCT

SEQ ID NO: 38 - protein, 11 aa, artificial sequence
  peptide unit
  SITE (1)..(1): D-amino acid
  SITE (2)..(2): L-amino acid
  misc_feature (5)..(5): any amino acid residue comprising -COOH
PPAWXLGELVW

SEQ ID NO: 39 - protein, 11 aa, artificial sequence
  peptide unit
  SITE (1)..(1): D-amino acid
  SITE (2)..(2): L-amino acid
PPAWDLGELVW

SEQ ID NO: 40 - protein, 11 aa, artificial sequence
  peptide unit
  SITE (1)..(1): D-amino acid
  SITE (2)..(2): L-amino acid
PPAWHLGELVW

SEQ ID NO: 41 - protein, 15 aa, artificial sequence
  peptide unit
  SITE (1)..(1): D-amino acid
  SITE (2)..(2): L-amino acid
  disulfid (4)..(14)
  misc_feature (7)..(7): any amino acid comprising -COOH
PPDCAWXLGELVWCT

SEQ ID NO: 42 - protein, 15 aa, artificial sequence
  peptide unit
  SITE (1)..(1): D-amino acid
  SITE (2)..(2): L-amino acid
  disulfid (4)..(14)
PPDCAWHLGELVWCT

SEQ ID NO: 43 - protein, 15 aa, artificial sequence
  peptide unit
  SITE (1)..(1): D-amino acid
  SITE (2)..(2): L-amino acid
  disulfid (4)..(14)
PPDCAWDLGELVWCT

SEQ ID NO: 44 - protein, 15 aa, artificial sequence
  peptide unit
  disulfid (1)..(15)
  disulfid (3)..(13)
  misc_feature (6)..(6): any amino acid residue comprising -COOH
CDCAWXLGELVWCTC

SEQ ID NO: 45 - protein, 15 aa, artificial sequence
  peptide unit
  disulfid (1)..(15)
  disulfid (3)..(13)
CDCAWHLGELVWCTC

SEQ ID NO: 46 - protein, 15 aa, artificial sequence
  peptide unit
  disulfid (1)..(15)
  disulfid (3)..(13)
CDCAWDLGELVWCTC

SEQ ID NO: 47 - protein, 13 aa, artificial sequence
  antibody binding moiety
  disulfid (2)..(12)
  binding (10)..(10): any amino acid
  misc_feature (10)..(10): X is an residue with COOH
  misc_feature (10)..(10): X is an amino acid residue
DCAWHLGELXWCT

SEQ ID NO: 48 - protein, 13 aa, artificial sequence
  antibody binding moiety
  disulfid (2)..(12)
  binding (6)..(6): any amino acid
  misc_feature (6)..(6): antibody binding moiety
  misc_feature (6)..(6): any amino acid residue
DCAWHXGELVWCT

SEQ ID NO: 49 - protein, 13 aa, artificial sequence
  antibody binding moiety
  disulfid (2)..(12)
  binding (8)..(8): X is any amino acid
  binding (8)..(8): antibody binding moiety
  misc_feature (8)..(8): X is any amino acid
DCAWHLGXLVWCT

SEQ ID NO: 50 - protein, 33 aa, artificial sequence
  antibody binding moiety
  Mod_Res (33)..(33): amidation
FNMQQQRRFYEALHDPNLNEEQRNAKIKSIRDD

SEQ ID NO: 51 - protein, 34 aa, artificial sequence
  antibody binding moiety
  disulfid (5)..(34)
FNMQCQRRFYEALHDPNLNEEQRNAKIKSIRDDC

SEQ ID NO: 52 - protein, 17 aa, artificial sequence
  antibody binding moiety
  disulfid (4)..(14)
RGNCAYHRGQLVWCTYH

SEQ ID NO: 53 - protein, 17 aa, artificial sequence
  antibody binding moiety
  disulfid (4)..(14)
  SITE (8)..(8): optional binding site
RGNCAYHKGQLVWCTYH

SEQ ID NO: 54 - protein, 17 aa, artificial sequence
  antibody binding moiety
  disulfid (4)..(14)
  SITE (5)..(5): optional binding site
RGNCKYHRGQLVWCTYH

SEQ ID NO: 55 - protein, 17 aa, artificial sequence
  antibody binding moiety
  disulfid (4)..(14)
  SITE (10)..(10): optional binding site
RGNCAWHRGKLVWCTYH

SEQ ID NO: 56 - protein, 17 aa, artificial sequence
  antibody binding moiety
  disulfid (4)..(14)
  SITE (5)..(5): optional binding site
RGNCKWHRGELVWCTYH

SEQ ID NO: 57 - protein, 17 aa, artificial sequence
  antibody binding moiety
  disulfid (4)..(14)
  site (5)..(5): optional binding site
RGNCKWHRGQLVWCTYH

SEQ ID NO: 58 - protein, 17 aa, artificial sequence
  antibody binding moiety
  disulfid (4)..(14)
  site (5)..(5): optional binding site
RGNCKYHLGELVWCTYH

SEQ ID NO: 59 - protein, 17 aa, artificial sequence
  antibody binding moiety
  disulfid (4)..(14)
  site (5)..(5): optional binding site
RGNCKYHLGQLVWCTYH

SEQ ID NO: 60 - protein, 13 aa, artificial sequence
  antibody binding moiety
  disulfid (2)..(12)
  site (3)..(3): optional binding site
DCKWHLGELVWCT

SEQ ID NO: 61 - protein, 13 aa, artificial sequence
  antibody binding moiety
  disulfid (2)..(12)
  site (3)..(3): optional binding site
DCKYHLGELVWCT

SEQ ID NO: 62 - protein, 13 aa, artificial sequence
  antibody binding moiety
  disulfid (2)..(12)
  site (3)..(3): optional binding site
DCKWHRGELVWCT

SEQ ID NO: 63 - protein, 13 aa, Artificial Sequence
  antibody binding moiety
  disulfid (2)..(12)
  site (3)..(3): optional binding site
DCKWHLGQLVWCT

SEQ ID NO: 64 - protein, 13 aa, artificial sequence
  antibody binding moiety
  disulfid (2)..(12)
  site (3)..(3): optional binding site
  site (3)..(3): optional binding site
DCKYHRGELVWCT

SEQ ID NO: 65 - protein, 13 aa, artificial sequence
  antibody binding moiety
  disulfid (2)..(12)
  site (3)..(3): optional binding site
DCKYHLGQLVWCT

SEQ ID NO: 66 - protein, 13 aa, artificial sequence
  antibody binding moiety
  disulfid (2)..(12)
  site (3)..(3): optional bindng site
DCKWHRGQLVWCT

SEQ ID NO: 67 - protein, 13 aa, artificial sequence
  antibody binding moiety
  disulfid (2)..(12)
  site (3)..(3): optional binding site
DCKYHRGQLVWCT

SEQ ID NO: 68 - protein, 34 aa, artificial sequence
  antibody binding moiety
  site (3)..(3): optional binding site
  disulfid (5)..(34)
FNKQCQRRFYEALHDPNLNEEQRNARIRSIRDDC

SEQ ID NO: 69 - protein, 34 aa, artificial sequence
  antibody binding moiety
  disulfid (5)..(34)
  site (31)..(31): optional binding site
FNMQCQRRFYEALHDPNLNEEQRNARIRSIKDDC

SEQ ID NO: 70 - protein, 34 aa, artificial sequence
  antibody binding moiety
  disulfid (5)..(34)
  site (20)..(20): optional binding site
FNMQCQRRFYEALHDPNLNKEQRNARIRSIRDDC

SEQ ID NO: 71 - protein, 34 aa, artificial sequence
  peptide unit
  disulfid (5)..(34)
FNMQCQRRFYEALHDPNLNEEQRNARIRSIRDDC

SEQ ID NO: 72 - protein, 17 aa, artificial sequence
  peptide unite
  disulfid (4)..(14)
  site (15)..(15): optional binding site
RGNCAWHLGQLVWCKYH

SEQ ID NO: 73 - protein, 17 aa, artificial sequence
  Peptide unit
  disulfid (4)..(14)
  site (15)..(15): optional binding site
RGNCAWHLGELVWCKYH

SEQ ID NO: 74 - protein, 17 aa, artificial sequence
  peptide unite
  disulfid (4)..(14)
  site (16)..(16): optional binding site
RGNCAYHLGQLVWCTKH

SEQ ID NO: 75 - protein, 17 aa, artificial sequence
  peptide unit
  disulfid (4)..(14)
  site (17)..(17): optional binding site
RGNCAYHLGQLVWCTYK

SEQ ID NO: 76 - protein, 17 aa, artificial sequence
  peptide unit
  disulfid (4)..(14)
  site (16)..(16): optional binding site
RGNCAYHRGQLVWCTKH

SEQ ID NO: 77 - protein, 34 aa, artificial sequence
  peptide unit
  site (1)..(1): optional binding site
  disulfid (5)..(34)
KNMQCQRRFYEALHDPNLNEEQRNARIRSIRDDC

SEQ ID NO: 78 - protein, 34 aa, artificial sequence
  peptide unit
  disulfid (5)..(34)
  site (7)..(7): optional binding site
FNMQCQKRFYEALHDPNLNEEQRNARIRSIRDDC

SEQ ID NO: 79 - protein, 34 aa, artificial sequence
  peptide unit
  disulfid (5)..(34)
  site (13)..(13): optional binding site
FNMQCQRRFYEAKHDPNLNEEQRNARIRSIRDDC

SEQ ID NO: 80 - protein, 34 aa, artificial sequence
  peptide unite
  disulfid (5)..(34)
  site (24)..(24): optional binding site
FNMQCQRRFYEALHDPNLNEEQRKARIRSIRDDC

SEQ ID NO: 81 - protein, 34 aa, artificial sequence
  peptide unit
  disulfid (5)..(34)
  site (6)..(6): optional binding site
FNMQCKRRFYEALHDPNLNEEQRNARIRSIRDDC

SEQ ID NO: 82 - protein, 34 aa, artificial sequence
  peptide unit
  disulfid (5)..(34)
  site (32)..(32): optional binding site
FNMQCQRRFYEALHDPNLNEEQRNARIRSIRKDC

SEQ ID NO: 83 - protein, 17 aa, artificial sequence
  peptide unit
  Mod-Res (1)..(1): acetylation
  disulfid (4)..(14)
  misc_feature (8)..(8): X is K or R
GPDCAYHXGELVWCTFH

SEQ ID NO: 84 - protein, 5 aa, artificial sequence
  Peptide tag
GGGGG

SEQ ID NO: 85 - protein, 5 aa, artificial sequence
  peptide tag
LPETG

SEQ ID NO: 86 - protein, 5 aa, artificial sequence
  peptide tag
  misc_feature (3)..(3): any amino acid
LPXTG

SEQ ID NO: 87 - protein, 5 aa, artificial sequence
  peptide tag
  misc_feature (3)..(3): any amino acid
  Non_Ter (5)..(5): any amino acid
LPXTG

SEQ ID NO: 88 - protein, 5 aa, artificial sequence
  peptide tag
  Non_Ter (5)..(5)
LPETG

SEQ ID NO: 89 - protein, 7 aa, artificial sequence
  reactive group
  misc_feature (6)..(7): any amino acid
LPETGXX

SEQ ID NO: 90 - protein, 43 aa, artificial sequence
  reactive group
GGSEQKLISEEDLGSGGGGSLPETGGGGSGGSSHHHHHHHHHH

SEQ ID NO: 91 - protein, 56 aa, artificial sequence; identical to SEQ ID NO: 4
  artificial compound
  Mod_Res (1)..(1): Acetylation
  Mod_Res (56)..(56): Amidation

SEQ ID NO: 92 - protein, 56 aa, artificial sequence; identical to SEQ ID NO: 4
  artificial compound
  Binding (1)..(1): Chemical group
  Mod_Res (56)..(56): Amidation

SEQ ID NO: 93 - protein, 56 aa, artificial sequence; identical to SEQ ID NO: 3
  artificial compound
  Mod_Res (1)..(1): Acetylation

SEQ ID NO: 94 - protein, 56 aa, artificial sequence; identical to SEQ ID NO: 3
  artificial compound
  Binding (1)..(1): Chemical group

SEQ ID NO: 95 - protein, 64 aa, artificial sequence; identical to SEQ ID NO: 6
  artificial compound
  Mod_Res (1)..(1): Acetylation

SEQ ID NO: 96 - protein, 64 aa, artificial sequence; identical to SEQ ID NO: 6
  artificial compound
  Binding (1)..(1): Chemical group

SEQ ID NO: 97 - protein, 56 aa, artificial sequence; identical to SEQ ID NO: 3
  artificial compound
  Mod_Res (56)..(56): Amidation

SEQ ID NO: 98 - protein, 56 aa, artificial sequence; identical to SEQ ID NO: 3
  artificial compound
  Mod_Res (1)..(1): Acetylation
  Mod_Res (56)..(56): Amidation

SEQ ID NO: 99 - protein, 56 aa, artificial sequence; identical to SEQ ID NO: 3
  artificial compound
  Binding (1)..(1): Chemical group
  Mod_Res (56)..(56): amidation

SEQ ID NO: 100 - protein, 64 aa, artificial sequence; identical to SEQ ID NO: 6
  artificial compound
  Mod_Res (64)..(64): Amidation

SEQ ID NO: 101 - protein, 64 aa, artificial sequence; identical to SEQ ID NO: 6
  artificial compound
  Mod_Res (1)..(1): acetylation
  Mod_Res (64)..(64): amidation

SEQ ID NO: 102 - protein, 64 aa, artificial sequence; identical to SEQ ID NO: 6
  artificial compound
  Binding (1)..(1): Chemical group
  Mod_Res (64)..(64): amidation

SEQ ID NO: 103 - protein, 56 aa, artificial sequence; identical to SEQ ID NO: 3
  artificial compound
  Mod_Res (1)..(1): Acetylation
  Binding (56)..(56): Chemical group

SEQ ID NO: 104 - protein, 56 aa, artificial sequence; identical to SEQ ID NO: 3
  artificial compound
  Binding (56)..(56): chemical group

SEQ ID NO: 105 - protein, 99 aa, artificial sequence
  artificial compound
  SITE (57)..(99): Reactive group
DKEEILNKIYEIMRLLDELGNAEASMRVSDLILEFMKKGDERLLEEAERLLEEVERGGSEQKLISEEDLGSGGGGSLPETGGGGSGGSSHHHHHHHHHH

SEQ ID NO: 106 - protein, 107 aa, artificial sequence
  artificial compound
  Site (65)..(107): reactive group
NDDELLMLVTDLVAEALLFAKDEEIKKRVFTLFELADKAYKNNDRDTLSKVVSELKELLERLQSGGSEQKLISEEDLGSGGGGSLPETGGGGSGGSSHHHHHHHHHH

SEQ ID NO: 107 - protein, 80 aa, artificial sequence
  artificial compound
  binding (1)..(1): Chemical group
DKEEILNKIYEIMRLLDELGNAEASMRVSDLILEFMKKGDERLLEEAERLLEEVERGGSEQKLISEEDLGSGGGGSLPET

SEQ ID NO: 108 - protein, 88 aa, artificial sequence
  artificial compound
  binding (1)..(1): Chemical group
NDDELLMLVTDLVAEALLFAKDEEIKKRVFTLFELADKAYKNNDRDTLSKVVSELKELLERLQSGGSEQKLISEEDLGSGGGGSLPET

SEQ ID NO: 109 - protein, 64 aa, artificial sequence
  Peptide unit
NDDELLMLVTDLVAEALLFAKDEEIKKRVFTLFELADKAYKNNDRDTLSKVVSELKELLERLQS

(19) United States
(12) Patent Application Publication
(10) Pub. No.: US 2024/0335555 A1
(43) Pub. Date: Oct. 10, 2024

(54) TECHNOLOGIES FOR PREVENTING OR TREATING INFECTIONS

(71) Applicant: Biohaven Therapeutics Ltd., New Haven, CT (US)

(72) Inventors: Wieslaw KAZMIERSKI, Raleigh, NC (US); Tetyana BERBASOVA, Hamden, CT (US); Luca RASTELLI, Madison, CT (US); Lawrence G. IBEN, Bethany, CT (US); Alexander BAYDEN, Hamden, CT (US); Anna BUNIN, Brooklyn, NY (US); Scott CONROY, Coventry, CT (US)

(21) Appl. No.: 18/263,492

(22) PCT Filed: Feb. 6, 2022
(86) PCT No.: PCT/US2022/015390

§ 371 Date: Jul. 28, 2023

Related U.S. Application Data
(60) Provisional application No. 63/151,785, filed on Feb. 21, 2021.
(60) Provisional application No. 63/146,584, filed on Feb. 6, 2021.

(51) Int. Cl. A61K 47/68; C07K 16/10

(52) U.S. Cl. CPC A61K 47/6841; A61K 47/6811; A61K 47/6849; C07K 16/1002

(57) ABSTRACT

Among other things, the present disclosure provides agents that can bind to viruses such as SARS-CoV-2 and/or cells infected thereby. In some embodiments, the present disclosure provides methods for preventing and/or treating conditions, disorders or diseases associated with SARS-CoV-2 infection. In some embodiments, the present disclosure provides methods for preventing and/or treating COVID-19.

### CROSS REFERENCE TO RELATED APPLICATION

This application is a National Stage application of PCT/US2022/015390, filed on Feb. 6, 2022, which claims priority to U.S. Provisional Application No. 63/146,584 filed Feb. 6, 2021 and U.S. Provisional Application No. 63/151,785 filed Feb. 21, 2021, the contents of which applications are incorporated herein their entireties by reference.

### SEQUENCE LISTING

The Instant Application contains a Sequence Listing which has been submitted electronically in ASCII format and is hereby incorporated by reference in its entirety. Said ASCII copy, created on Apr. 12, 2024 is named “BHL0001US3_ST25” and is 49,485 bytes in size. The Sequence Listing does not go beyond the disclosure in the application as filed.

### BACKGROUND

Coronaviruses are a diverse group of viruses that can infect many animals including humans, and can cause mild to severe respiratory infections in humans. Severe acute respiratory syndrome coronavirus 2 (SARS-CoV-2) is a highly transmissible coronavirus. It has been reported to cause a pandemic of acute respiratory disease, named “coronavirus disease 2019” (COVID-19), which threatens human health and public safety.

### TECHNICAL FIELD

In some embodiments, the present disclosure provides chemical and biological technologies. In some embodiments, provided technologies are useful for preventing and/or treating various conditions, disorders or diseases.

### SUMMARY

Among other things, the present disclosure provides technologies (e.g., agents, compositions, methods, etc.) for preventing and/or treating conditions, disorders or diseases associated with SARS-CoV-2. In some embodiments, a condition, disorder or disease is Coronavirus disease 2019, COVID-19. In some embodiments, provided technologies disrupts or reduces interaction between a cell and a SARS-CoV-2 virus. In some embodiments, provided technologies disrupts or reduces interactions between a spike protein (S protein) of SARS-CoV-2 and a receptor, e.g., ACE2, or a cell. In some embodiments, provided technologies disrupting or reducing an infection of a SARS-CoV-2 virus of a cell. In some embodiments, provided technologies inhibit, kill or remove SARS-CoV-2 viruses. In some embodiments, provided technologies inhibit, kill or remove cells infected by SARS-CoV-2 viruses. In some embodiments, provided technologies inhibit, kill or remove a cell expressing a spike protein of SARS-CoV-2 or a fragment thereof. In some embodiments, a cell is a mammalian cell that can be infected by SARS-CoV-2. In some embodiments, a cell is a human cell.

In some embodiments, an agent of present disclosure comprises a moiety that can binds to a target, e.g., a target binding moiety as described herein (e.g., a moiety that can bind to or recognize a SARS-CoV-2 virus, in some embodiments, through a spike protein, and in some embodiments, through RBD of a spike protein), and a second moiety. In some embodiments, a second moiety can promote, induce, and/or recruit immune activities. In some embodiments, a second moiety is or comprises an antibody moiety. In some embodiments, a second moiety is or comprises an antibody binding moiety.

In some embodiments, the present disclosure provides an agent comprising:
an antibody moiety,
a target binding moiety, and
optionally a linker moiety linking an antibody moiety and a target binding moiety.

In some embodiments, the present disclosure provides an agent having the structure of formula M-I:

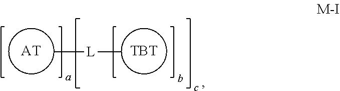

or a pharmaceutically acceptable salt thereof, wherein:
each of a, b and c is independently 1-200;
each AT is independently an antibody moiety;
L is a linker moiety; and
each TBT is independently a target binding moiety.

In some embodiments, the present disclosure provides an agent having the structure of formula M-II:

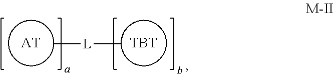

or a pharmaceutically acceptable salt thereof, wherein:
each of a and b is independently 1-200;
each AT is independently an antibody moiety;
L is a linker moiety; and
each TBT is independently a target binding moiety.

In some embodiments, a is 1. In some embodiments, b is 1. In some embodiments, c is 1. In some embodiments, c is 2. In some embodiments, for agents in a composition, c is a ratio of target binding moieties and antibody moieties as described herein, e.g., in some embodiments, about 0.1-6, 0.5-2.5, 1-2, 1.5-2, about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5 or 3, etc. As appreciated by those skilled in the art, various technologies can be utilized to prepare provided as described herein. In some embodiments, the present disclosure provides technologies that can provide selective conjugation at certain residues of antibody moieties and/or provide narrower or specific ratio ranges for target binding moieties and antibody moieties. In some embodiments, provided compositions are particularly homogenous.

In some embodiments, an antibody moiety is a moiety of an antibody in an IVIG composition. In some embodiments, an antibody moiety is a moiety of an antibody in a polyclonal antibody composition. In some embodiments, an antibody moiety is a moiety of an antibody in a monoclonal antibody composition. Among other things, IVIG is readily available and is approved for treating several diseases. In some embodiments, antibody moieties are a subject's own IgG or fragments thereof. In some embodiments, antibody moieties are a pooled IgG preparation, e.g., certain IVIG preparations, or fragments thereof. Various antibody moieties can be utilized in accordance with the present disclosure. Certain antibody moieties are described herein as examples.

In some embodiments, the present disclosure provides an agent comprising:
an antibody binding moiety,
a target binding moiety, and
optionally a linker moiety linking an antibody binding moiety and a target binding moiety.

In some embodiments, the present disclosure provides an agent has the structure of formula I:

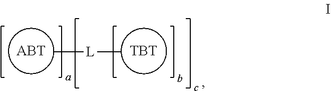

or a pharmaceutically acceptable salt thereof, wherein:
each of a, b and c is independently 1-200;
each ABT is independently an antibody binding moiety;
L is a linker moiety; and
each TBT is independently a target binding moiety.

In some embodiments, a is 1. In some embodiments, b is 1. In some embodiments, c is 1. In some embodiments, each of a, b and c is 1.

In some embodiments, an agent or antibody binding moiety can recruit antibodies. In some embodiments, an agent or antibody binding moiety can recruit different types of antibodies. In some embodiments, recruited antibodies or antibody moieties of agents can recruit immune cells. In some embodiments, recruited antibodies or antibody moieties of agents comprise IgG or a fragment thereof. In some embodiments, they comprise IgG1 or a fragment thereof. In some embodiments, they comprise IgG2 or a fragment thereof. In some embodiments, they comprise IgG3 or a fragment thereof. In some embodiments, they comprise IgG4 or a fragment thereof. In some embodiments, an antibody or a fragment thereof is or comprises a Fc region. In some embodiments, recruited antibodies or antibody moieties of agents interact with hFcγRIIIA. In some embodiments, they interact with hFcγRIIIA on macrophages. In some embodiments, they interact with hFcγRIIA. In some embodiments, they interact with hFcγRIIA on dendritic cells. In some embodiments, they recruit dendritic cells. In some embodiments, they recruit NK cells. In some embodiments, they recruit macrophages.

In some embodiments, a moiety, e.g., a target binding moiety described herein, targets SARS-CoV-2. In some embodiments, a moiety or agent binds to a spike protein of a SARS-CoV-2 virus. In some embodiments, a moiety or agent binds to spike receptor binding domain (RBD). In some embodiments, a moiety or agent competes with binding of a spike protein (or complexes thereof such as trimers thereof) to human angiotensin-converting enzyme 2 (ACE2) receptor. In some embodiments, moieties are or comprise -(Xaa)y- as described herein. In some embodiments, an agent has the structure of formula T-I,

R^{CN}-(Xaa)y-R^{CC}, T-I

or a salt form thereof, wherein:
R^{CN} and R^{CC} is independently R^{C};
each Xaa is independently a residue of an amino acid or an amino acid analog;
y is 5-50;
each R^{c} is independently -L^{a}-R′;
each L^{a} is independently a covalent bond, or an optionally substituted bivalent group selected from C₁-C₅₀ aliphatic or C₁-C₅₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, a moiety, e.g., a target binding moiety targeting SARS-CoV-2 (e.g., through binding to a spike protein), is linked to another moiety, e.g., an antibody moiety, an antibody binding moiety, etc., optionally through a linker moiety, to provide agents that can recruit immune activities (e.g., antibodies, immune cells, etc.). In some embodiments, linking is through N-terminus, C-terminus, and/or a side chain of a peptide moiety (e.g., of a target binding moiety which is or comprises a peptide moiety).

In some embodiments, a moiety, e.g., a target binding moiety, is a moiety of an agent having the structure of T-I or a salt thereof (e.g., as appreciated by those skilled in the art, by removing one or more —H to form a monovalent, bivalent or polyvalent moiety). In some embodiments, a moiety has the structure of —(R^{CN}-(Xaa)y-R^{CC}).

In some embodiments, a target binding moiety is of R^{CN}-(Xaa)y-R^{CC}. In some embodiments, a target binding moiety is —(R^{CN}-(Xaa)y-R^{CC})

In some embodiments, a target binding moiety or -(Xaa)y- is or comprises a sequence that is or shares at least about 50%, 55%, 60%, 65%, 70%, 75%, 80%, 85% 90%, 91%, 92%, 93%, 94%, 95%, 96%, 97%, 98% or 99% identity with a sequence selected from the below, or is or comprises a sequence selected from the below with 0-10 deletions 0-10 additions and 0-10 replacements:

(SEQ ID NO: 1)
DEDLEELERLYRKAEEVAKEAKDASRRGDDERAKEQMERAMRLFDQVFE
LAQELQEKQTDGNRQKATHLDKAVKEAADELYQRVR,
(SEQ ID NO: 2)
ELEEQVMHVLDQVSELAHELLHKLTGEELERAAYFNWWATEMMLELIKS
DDEREIREIEEEARRILEHLEELARK,
(SEQ ID NO: 3)
DKEWILQKIYEIMRLLDELGHAEASMRVSDLIYEFMKKGDERLLEEAER
LLEEVER,
(SEQ ID NO: 4)
DKEEILNKIYEIMRLLDELGNAEASMRVSDLILEFMKKGDERLLEEAER
LLEEVER,
(SEQ ID NO: 5)
SDDEDSVRYLLYMAELRYEQGNPEKAKKILEMAEFIAKRNNNEELERLV
REVKKRL,
(SEQ ID NO: 6)
NDDELHMLMTDLVYEALHFAKDEEIKKRVFQLFELADKAYKNNDRQKLE
KVVEELKELLERLLS,
(SEQ ID NO: 7)
QREKRLKQLEMLLEYAIERNDPYLMFDVAVEMLRLAEENNDERIIERAK
RILEEYE,
(SEQ ID NO: 8)
SLEELKEQVKELKKELSPEMRRLIEEALRFLEEGNPAMAMMVLSDLVYQ
LGDPRVIDLYMLVTKT,
(SEQ ID NO: 9)
DREQRLVRFLVRLASKFNLSPEQILQLFEVLEELLERGVSEEEIRKQLE
EVAKELG,
(SEQ ID NO: 10)
DDDIRYLIYMAKLRLEQGNPEEAEKVLEMARFLAERLGMEELLKEVREL
LRKIEELR,
and
(SEQ ID NO: 11)
PIIELLREAKEKNDEFAISDALYLVNELLQRTGDPRLEEVLYLIWRALK
EKDPRLLDRAIELFER.

In some embodiments, a selected sequence is DKEWILQKIYEIMRLLDELGHAEASMRVSDLIYEFMKKGDERLLEEAERLLEEVER (SEQ ID NO:3). In some embodiments, a selected sequence is DKEEILNKIYEIMRLLDELGNAEASMRVSDLILEFMKKGDERLLEEAERLLEEVER (SEQ ID NO:4). In some embodiments, a selected sequence is NDDELHMLMTDLVYEALHFAKDEEIKKRVFQLFELADKAYKNNDRQKLEKVVEELKELLERLL S (SEQ ID NO:6).

As described herein, in some embodiments, provided technologies comprise antibody moieties. In some embodiments, provided agents comprise antibody moieties linked to target binding moieties optionally through linker moieties (in some embodiments, such reagents referred to as MATE agents). In some embodiments, provided technologies comprise antibody binding moieties linked to target binding moieties optionally through linker moieties (in some embodiments, such reagents referred to as ARM agents). Without the intention to be bound by any theory, in some embodiments, provided technologies can recruit antibodies to an entity expressing a SARS-CoV-2 spike protein (unless otherwise indicated, including mutants thereof (e.g., those in viruses and/or infected cells)) or a fragment thereof (e.g., a SARS-CoV-2 virus, a cell infected by a SARS-CoV-2 virus, etc.). In some embodiments, antibody recruitment is or comprises binding of an agent comprising an antibody moiety and a target binding moiety. In some embodiments, antibody recruitment is or comprises binding of an agent comprising an antibody binding moiety, which can bind to antibodies, and a target binding moiety. In some embodiments, recruited antibodies reduces, inhibits or prevents interaction of SARS-CoV-2 viruses with other cells (e.g., mammalian cells that can be infected), in some embodiments, through disrupting, inhibiting or preventing interactions between SARS-CoV-2 spike proteins and cell proteins, e.g., receptors such as ACE2. In some embodiments, recruited antibodies can induce, recruit, promote, encourage, or enhance one or more immune activities to inhibit, suppress, kill, or remove SARS-CoV-2 viruses and/or celled infected thereby. In some embodiments, as appreciated by those skilled in the art, recruited antibodies recruit various types of immune cells.

In some embodiments, provided agents recruit antibodies or comprise antibody moieties. In some embodiments, provided agents bind spike proteins (e.g., at S1/2 domain, the RBD domain, etc.) on virus surfaces, preventing viruses from binding to cells (e.g., preventing viruses from binding to ACE2 receptors on human cells). In some embodiments, provided technologies inhibit viruses from infecting cells. In some embodiments, provided technologies neutralize SARS-CoV-2 viruses. In some embodiments, provided technologies provide direct virus neutralization and/or killing. In some embodiments, provided technologies block virus entry into cells (e.g., human cells).

In some embodiments, provided technologies recruit antibodies, or comprise antibody moieties, that can interact with various Fc receptors, recruit various effector cells and provide various immune activities. In some embodiments, antibodies or antibody moieties effectively interact with FcγRII and/or FcγRIII, e.g., those expressed by macrophages, NK cells, etc. In some embodiments, recruited antibodies or agents comprising antibody moieties recruit macrophages. In some embodiments, recruited antibodies or agents comprising antibody moieties recruit NK cells. In some embodiments, recruited antibodies or agents comprising antibody moieties recruit macrophages and NK cells. In some embodiments, agents of the present disclosure provides inhibition, killing, and removal of SARS-CoV-2 viruses and/or cells infected thereby. Recruited immune cells can provide various immune activities. In some embodiments, macrophages can remove viral particles, e.g., through phagocytosis. In some embodiments, NK cells can kill infected cells. In some embodiments, provided technology provide immune-mediated virus killing (of viruses and/or cells infected thereby).

In some embodiments, provided technologies (e.g., through antibody moieties of provided agents or recruited antibodies by provided agents) can recruit antigen presenting cells, e.g., dendritic cells. In some embodiments, recruited dendritic cells express FcγRII. In some embodiments, provided technologies can deliver viral proteins (e.g., expressed by viruses and/or infected cells) to antigen presenting cells. In some embodiments, provided technologies can provide antigen presentation to various immune cells, e.g., B cell, T cells, etc. In some embodiments, provided technologies can induce, recruit, promote, facilitate, encourage, or enhance priming and activation of immune memory cells (e.g., B-cells and T-cells). In some embodiments, provided technologies can instill long-term immunity (e.g., in some embodiments, like one or more aspects of a vaccine). In some embodiments, provided technologies provide long-term vaccination effect.

In some embodiments, provided agents, e.g., those comprising antibody moieties, bind to FcRn. In some embodiments, provided agents comprising antibody moieties bind to FcRn for antibody recycle and/or prolonged half-life.

In some embodiments, an immune activity is associated with immune cells. In some embodiments, an immune activity is associated with macrophages. In some embodiments, immune cells are or comprise macrophages. In some embodiments, an immune activity is associated with NK cells. In some embodiments, immune cells are or comprise NK cells. In some embodiments, immune cells are engineered cells. In some embodiments, immune cells are prepared in vitro. For example, in some embodiments, NK cells are or comprise engineered cells. In some embodiments, NK cells are or comprise autologous NK cells. In some embodiments, NK cells are collected, expanded and/or stored autologous NK cells. In some embodiments, NK cells are or comprise allogeneic NK cells. In some embodiments, NK cells are or comprises peripheral blood-derived NK cells. In some embodiments, NK cells are or comprises cord blood-derived NK cells. In some embodiments, provided technologies comprise immune cells in addition to provided agents. In some embodiments, immune cells are administered concurrently with provided agents; in certain embodiments, in the same composition. In some embodiments, immune cells are administered prior to or subsequently to provided agents.

In some embodiments, the present disclosure provides a method for treating a condition, disorder or disease associated with SARS-CoV-2 infection, comprising administering to a subject suffering therefrom a provided agent or composition. In some embodiments, the present disclosure provides a method for treating COVID-19, comprising administering to a subject suffering therefrom a provided agent or composition. In some embodiments, the present disclosure provides a method for inhibiting, killing or removing a SARS-CoV-2, comprising contacting a SARS-CoV-2 with a provided agent or composition. In some embodiments, the present disclosure provides a method for disrupting, reducing or preventing an interaction between a cell and a SARS-CoV-2, comprising contacting a SARS-CoV-2 with a provided agent or composition. In some embodiments, the present disclosure provides a method for disrupting, reducing or preventing an infection of a SARS-CoV-2 of a cell, comprising contacting a SARS-CoV-2 with a provided agent or composition. In some embodiments, the present disclosure provides a method for inhibiting, killing or removing a cell infected by a SARS-CoV-2, comprising contacting the cell with a provided agent or composition. In some embodiments, a cell is a mammalian cell. In some embodiments, a cell is a human cell. In some embodiments, provided agents or compositions are utilized in amounts effective to provide desired effects. As described herein, in some embodiments, immune cells, such as various NK cells (e.g., allogeneic NK cells, peripheral blood-derived NK cells, MG4101 NK cells, CB-NK NK cells, cord blood-derived NK cells, etc.), may be utilized together with provided agents and/or compositions, and may be administered prior to, concurrently with, or subsequently to provided agents and/or compositions.

In some embodiments, the present disclosure provides pharmaceutical compositions comprising or delivering a provided agent or a pharmaceutically acceptable salt thereof and a pharmaceutically acceptable carrier. In some embodiments, provided technologies are administered to subjects in pharmaceutical compositions.

Provided technologies can provide various benefits and advantages. In some embodiments, provided agents (e.g., certain MATE and/or ARM agents) can be produced through chemical synthesis with both speed and quantity. In some embodiments, provided agents are more stable that therapeutic agents such as certain types of antibodies and/or serums, and can be readily stored and distributed in complex global logistical networks. In some embodiments, provided agents are sufficiently stable and do not require cold-chain distribution. In some embodiments, provided agents can be stockpiled (which can be particularly useful for fighting pandemics). In some embodiments, provided agents (e.g., certain ARM agents) are smaller in size than many therapeutic antibodies, and can penetrate and be delivered to locations that cannot be readily reached by therapeutic antibodies. In some embodiments, provided technologies can quickly convert antibodies into agents (e.g., MATE agents) targeting targets such as SARS-CoV-2. In some embodiments, readily available antibody preparations, e.g., IVIG, can be utilized and can provide many advantages such as speed, safety, quality and/or low cost of goods. In some embodiments, provided agents can penetrate tissues more quickly and/or at higher levels than other agents. In some embodiments, provided agents provide suitable safety profile, and in some embodiments, have been demonstrated to be safer in animal models (e.g., monkeys) than certain therapeutic monoclonal antibodies. In some embodiments, provided agents can be safely administered at higher concentrations compared to certain monoclonal antibodies.

Among other things, the present disclosure demonstrates that provided technologies can provide high binding affinity and/or virus neutralization.

### BRIEF DESCRIPTION OF THE DRAWING

These and/or other aspects will become apparent and more readily appreciated from the following description of the embodiments, taken in conjunction with the accompanying drawings in which:

FIG. 1. Reduced SDS-PAGE assessment of certain I-24 and I-25 preparation. From left to right: ladder, I-24 and I-25. 4-12% NuPAGE gel. 150V 1.5 hours. MOPS running buffer.

FIG. 2. Certain data of I-26 and I-27. From left to right: ladder, IVIG, reduced I-26, reduced I-27, reduced IVIG, empty, I-26, and I-27. Peptide: I-24 or I-25. Markers that ran off gel: 15 kDa and 10 kDa.

FIG. 3. Certain data of I-24 and I-28. From left to right: ladder, IVIG (Gamunex-C), I-24, empty, I-28-1 (Gamunex), I-28-2 (Flebogamma), cleaned up MATEs. The lowest arrow indicates low molecular agents, which might include agents comprising unconjugated target binding moiety peptide.

FIG. 4. Certain results for I-28 and I-40. From left to right: I-40 (IVIG+I-39) and I-28-3.

FIG. 5. Schematic showing the preparation of a MATE compound. The exemplified compound is I-36, the shown as an IVIG congjugate3 in Table I, below.

FIG. 6. LC-MS spectra for I-20 coupled to Ig. CUTAQUIG was used as the Ig. The peak at 31599 mass represents the Fc domain of antibody conjugated to I-20, forming I-36. The peak at 23 1.757 is unconjugated antibody. The LC-MS spectra for I-36 having a BAR (binder/antibody ratio) of 1.54 (1.50 intended is shown. The LC-MS sample was prepared by incubating 100 μg protein (comprised of conjugate and unconjugated Ig) with 1 μl each of the enzymes IdeZ and PNGase F at 37° C. for 1 hour.

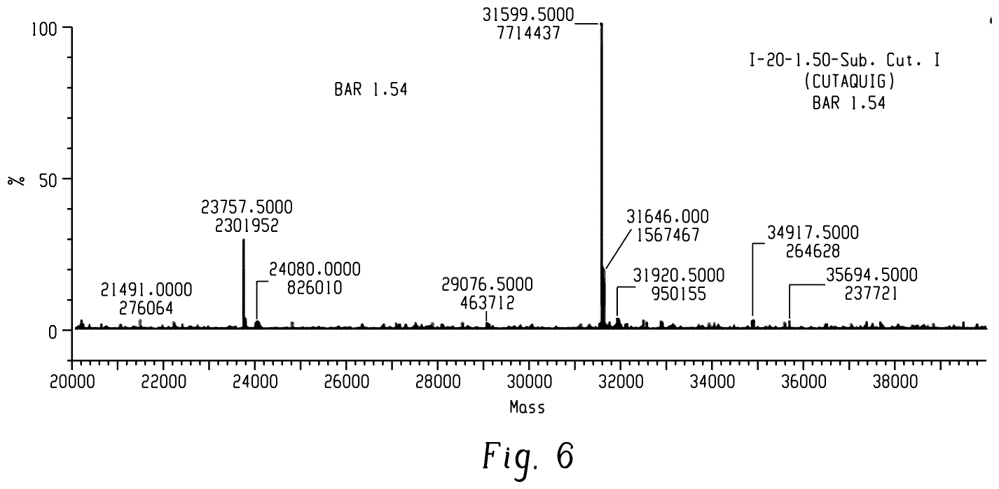

FIG. 7. Analysis of IGSC-I-20 conjugates by SEC-HPLC. The column was a TSKgel Super SW3000 column (Tosoh Bioscience, King of Prussia, PA). The top trace shows unconjugated CUTAQUIG in HEPES buffer. Conjugation with increasing ratios of I-20 shows the appearance of conjugated species. Chromatography was performed using a PBS, pH 6.8 mobile phase, 0.35 mL/min flow rate, isocratic gradient, 18 minutes, and 280 nm UV detection.

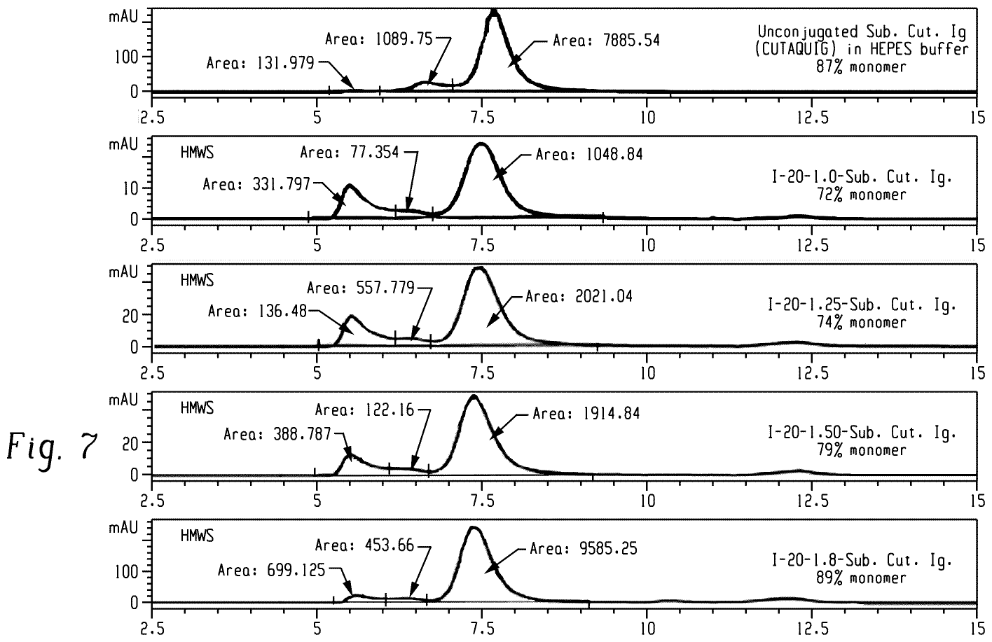

FIG. 8. HPLC of I-36 reaction mixture (I-20 conjugated to IVIG). Peak 1 is unknown, Peak 2 is unconjugated antibody, peak 4/5 is conjugated antibody with a drug/antibody ratio of 1 and peak 6 is conjugated antibody with a drug/antibody ratio of 2. Peak 9 is excess MATE reagent (I-20). Peak identifications were determined via mass spectroscopy of the isolated peaks. The HPLC trace was obtained using an Agilent 1260 quat pump HPLC system. Solvent A was 0.05% TFA in water, Solvent B was 0.05% TFA in MeCN. The gradient was run at a column temperature of 80° C., 20 μl injection volume with a flow rate of 1 ml/min. The column was a HALO 1000A diphenyl, 10×100 mm, 2.7 μm column, P/N 92710-626.

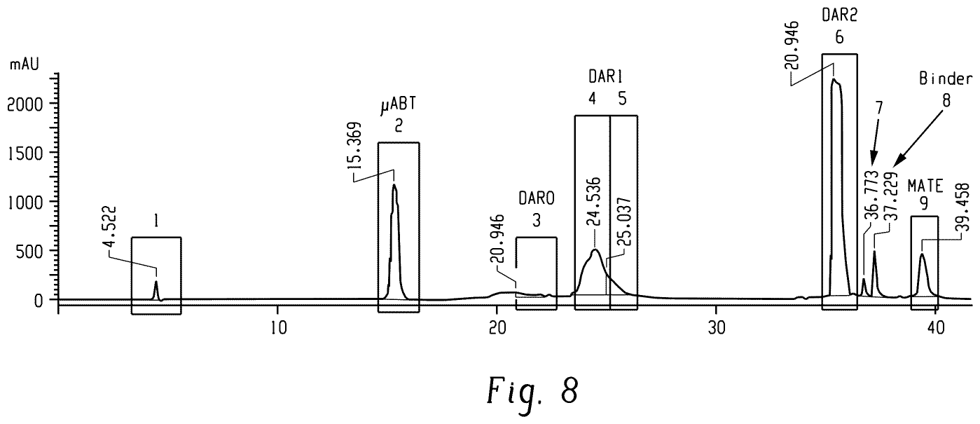

### DETAILED DESCRIPTION OF CERTAIN EMBODIMENTS

### 1. General Description of Certain Embodiments

In some embodiments, the present disclosure provides agents, e.g., antibody conjugates (e.g., agents comprising antibody moieties such as MATE agents (or MATEs)), antibody-recruiting molecules (ARM agents (or ARMs)), etc., that comprise target binding moieties that can bind to entities expressing SARS-CoV-2 spike protein or a fragment thereof (e.g., SARS-CoV-2 viruses and cells infected thereby). In some embodiments, provided agents, e.g., ARMs, comprise universal antibody binding moieties that can bind to antibodies with different Fab structures. In some embodiments, the present disclosure provides agents, e.g., ARMs, MATEs, etc., that comprises antibody binding moieties that bind to antibodies, e.g., Fc regions of antibodies, and such binding of antibodies do not interfere one or more immune activities of the antibodies, e.g., interaction with Fc receptors (e.g., CD16a), recruitment of effector cells like NK cells for ADCC, macrophage for ADCP, etc. As those skilled in the art will appreciate, provided technologies (agents, compounds, compositions, methods, etc.) of the present disclosure can provide various advantages, for example, provided technologies can utilize antibodies having various Fab regions in the immune system to avoid or minimize undesired effects of antibody variations among a patient population, can trigger, and/or enhance, immune activities toward targets, e.g., killing target entities such as SARS-CoV-2 viruses and cells infected thereby. In some embodiments, provided technologies can target one or more or all variants of SARS-CoV-2. In some embodiments, provided technologies can target one or more other virus that express entities that provided technologies recognize and bind to.

In some embodiments, provided technologies are useful for reducing, suppressing, inhibiting, blocking or preventing interactions of SARS-CoV-2 viruses with cells, e.g., those may be infected. In some embodiments, provided technologies are useful for reducing, suppressing, inhibiting, blocking or preventing infection of cells, tissues, organs, or subjects by SARS-CoV-2 viruses. In some embodiments, provided technologies are useful for modulating immune activities against targets (e.g., viruses, infected cells, etc.) expressing a SARS-CoV-2 spike protein or a fragment thereof. In some embodiments, technologies of the present disclosure are useful for recruiting antibodies to targets, particularly those expressing a SARS-CoV-2 spike protein or a fragment thereof. In some embodiments, provided agents can inhibit protein activities and/or interactions, e.g., those of a spike protein (e.g., expressed by a SARS-CoV-2 or a cell infected thereby). In some embodiments, a target binding moiety is an inhibitor moiety.

In some embodiments, the present disclosure provide an agent comprising:
an antibody moiety,
a target binding moiety which can bind a SARS-CoV-2 spike protein or a fragment thereof, and optionally a linker moiety.

In some embodiments, provided agents comprise one and only one antibody moiety. In some embodiments, provided agents comprise two or more antibody moieties. In some embodiments, provided agents comprise one and only one target binding moiety. In some embodiments, provided agents comprise two and only two target binding moieties. In some embodiments, provided agents comprise two or more target binding moieties. In some embodiments, target binding moieties are selectively linked to antibody binding moieties through particular amino acid residues. In some embodiments, target binding moieties are selectively linked to antibody binding moieties through amino acid residues at particular positions. In some embodiments, target binding moieties are selectively linked to antibody binding moieties through amino acid residues at one or two particular positions. In some embodiments, target binding moieties are selectively linked to antibody binding moieties through amino acid residues at a single particular position. Certain particular positions are as described herein, e.g., K246 and K248 of an IgG1 heavy chain and amino acid residues corresponding thereto, K251 and K253 of an IgG2 heavy chain and amino acid residues corresponding thereto, K239 and K241 of an IgG4 heavy chain and amino acid residues corresponding thereto, etc.

In some embodiments, the present disclosure provide an agent comprising:
an antibody binding moiety,
a target binding moiety which can bind a SARS-CoV-2 spike protein or a fragment thereof, and optionally a linker moiety, wherein the antibody binding moiety can bind to two or more antibodies which have different Fab regions.

In some embodiments, the present disclosure provide an agent comprising:
an antibody binding moiety,
a target binding moiety which can bind a SARS-CoV-2 spike protein or a fragment thereof, and optionally a linker moiety, wherein the antibody binding moiety can bind to two or more antibodies toward different antigens.

In some embodiments, provided agents comprise one and only one antibody binding moiety. In some embodiments, provided agents comprise two or more antibody binding moieties. In some embodiments, provided agents comprise one and only one target binding moiety. In some embodiments, provided agents comprise two or more target binding moieties.

An antibody binding moiety may interact with any portion of an antibody. In some embodiments, an antibody binding moiety binds to an Fc region of an antibody. In some embodiments, an antibody binding moiety binds to a conserved Fc region of an antibody. In some embodiments, an antibody binding moiety binds to an Fc region of an IgG antibody. As appreciated by those skilled in the art, various antibody binding moieties, linkers, and target binding moieties can be utilized in accordance with the present disclosure.

In some embodiments, the present disclosure provides antibody binding moieties and/or agents (e.g., compounds of various formulae described in the present disclosure, ARM molecules of the present disclosure, etc.) comprising antibody binding moieties that can bind to a Fc region that is bound to Fc receptors, e.g., FcγRIIIa, CD16a, etc. In some embodiments, provided moieties and/or agents comprising antibody binding moieties that bind to a complex comprising an Fc region and an Fc receptor.

In some embodiments, the present disclosure provides a complex comprising:
an agent comprising:
   an antibody binding moiety,
   a target binding moiety, and
   optionally a linker moiety,
an Fc region, and
an Fc receptor.

In some embodiments, an Fc region is an Fc region of an endogenous antibody of a subject. In some embodiments, an Fc region is an Fc region of an exogenous antibody. In some embodiments, an Fc region is an Fc region of an administered agent. In some embodiments, an Fc receptor is of a diseased cell in a subject.

In some embodiments, the present disclosure provides agents having a structure of:

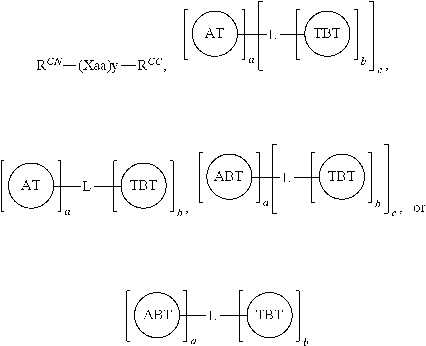

or a salt thereof. In some embodiments, an agent has the structure of

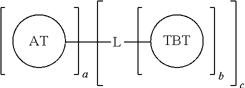

or a salt thereof. In some embodiments, an agent has the structure of

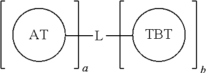

or a salt thereof. In some embodiments, an agent has the structure of

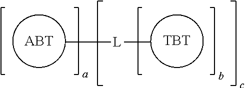

or a salt thereof. In some embodiments, an agent has the structure of

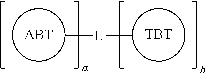

or a salt thereof. In some embodiments, each target binding moiety independently has the structure of —(R^{CN}-(Xaa)y-R^{CC}) or salt form thereof.

In some embodiments, a target binding moiety comprises one or more amino acid residues. In some embodiments, a target binding moiety is or comprises a peptide moiety. In some embodiments, a target binding moiety comprises one or more natural amino acid residues. In some embodiments, a target binding moiety comprises one or more unnatural natural amino acid residues. In some embodiments, a target binding moiety comprises an alpha-helical structure. In some embodiments, a target binding moiety comprises two alpha-helical structures. In some embodiments, a target binding moiety comprises three alpha-helical structures. In some embodiments, a target binding moiety comprises four alpha-helical structures.

In some embodiments, target binding moieties of an agent share a common or the same amino acid sequence. In some embodiments, target binding moieties of an agent share the same amino acid sequence. In some embodiments, target binding moieties of an agent have the same structure. In some embodiments, target binding moieties of a plurality of agents share a common or the same amino acid sequence. In some embodiments, target binding moieties of a plurality of agents share the same amino acid sequence. In some embodiments, target binding moieties of a plurality of agents have the same structure.

In some embodiments, an antibody binding moiety is a universal antibody binding moiety.

In some embodiments, an antibody binding moiety comprises one or more amino acid residues. In some embodiments, an antibody binding moiety is or comprises a peptide moiety. In some embodiments, an antibody binding moiety is or comprises a cyclic peptide moiety. In some embodiments, such antibody binding moiety comprises one or more natural amino acid residues. In some embodiments, such antibody binding moiety comprises one or more unnatural natural amino acid residues.

In some embodiments, an antibody-binding moiety is a cyclic peptide moiety. In some embodiments, an antibody binding moiety is or comprises

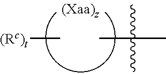

or a salt form thereof. In some embodiments, an antibody binding moiety is

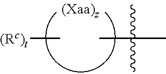

or a salt form thereof. In some embodiments, each antibody binding moiety in an agent independently is or comprises

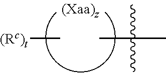

or a salt form thereof. In some embodiments, each antibody binding moiety in an agent is independently

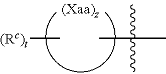

or a salt form thereof. In some embodiments, each antibody binding moiety in an agent is of the same antibody binding moiety or a salt thereof.

In some embodiments, the present disclosure provides a compound of formula I-a:

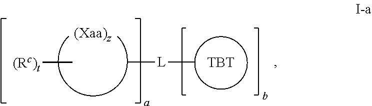

or a salt thereof, wherein:
each Xaa is independently a residue of an amino acid or an amino acid analog;
t is 0-50;
z is 1-50;
L is a linker moiety;
TBT is a target binding moiety;
each R^{c} is independently -L^{a}-R′;
each of a and b is independently 1-200;
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, a is 1. In some embodiments, b is 1. In some embodiments, a is 1 and b is 1, and a compound of formula I-a has the structure of

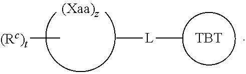

In some embodiments, each residue, e.g., Xaa, is independently a residue of an amino acid or an amino acid analog, wherein the amino acid or the amino acid analog has the structure of H-L^{a1}-L^{a1}-C(R^{a2})(R^{a3})-L^{a2}-L^{a2}-H or a salt thereof. In some embodiments, an amino acid has the structure of NH(R^{a1})-L^{a1}-C(R^{a2})(R^{a3})-L^{a2}-COOH or a salt thereof. In some embodiments, an amino acid analog has the structure of H-L^{a1}-L^{a1}-C(R^{a2})(R^{a3})-L^{a2}-L^{a2}-H or a salt thereof. In some embodiments, in such an amino acid analog, the first -L^{a1}-(bonded to —H in the formula) is not —N(R^{a1})— (e.g., is optionally substituted bivalent C₁₋₆ aliphatic). In some embodiments, in H-L^{a1}-L^{a1}-L^{a1}-L^{a1}- bonds to the —H through an atom that is not nitrogen. In some embodiments, in -L^{a2}-L^{a2}-H, —L^{a2}-L^{a2}- is not bonded to the —H through —C(O)O—. In some embodiments, each residue, e.g., each Xaa in formula I-a, is independently a residue of an amino acid having the structure of formula A-I.

In some embodiments, each Xaa independently has the structure of -L^{a1}-L^{a1}-C(R^{a2})(R^{a3})-L^{a2}-L^{a2}-. In some embodiments, each Xaa independently has the structure of -L^{aX1}-L^{a1}-C(R^{a2})(R^{a3})-L^{a2}-L^{aX2}-, wherein L^{aX1} is optionally substituted —NH—, optionally substituted —CH₂—, —N(R^{a1})—, or —S—, L^{aX2} is optionally substituted —NH—, optionally substituted —CH₂—, —N(R^{a1})— or —S—, and each other variable is independently as described herein. In some embodiments, L^{aX1} is optionally substituted —NH—, or —N(R^{a1})—. In some embodiments, L^{aX1} is optionally substituted —CH₂—, or —S—. In some embodiments, L^{aX2} is optionally substituted —NH—, optionally substituted —CH₂—, —N(R^{a1})—, or —S—. In some embodiments, optionally substituted —CH₂— is —C(O)—. In some embodiments, optionally substituted —CH₂— is not —C(O)—. In some embodiments, L^{aX2} is —C(O)—. In some embodiments, each Xaa independently has the structure of —N(R^{a1})-L^{a1}-C(R^{a2})(R^{a3})-L^{a2}-CO—.

In many embodiments, two or more residues, e.g., two or more Xaa residues, are linked together such that one or more cyclic structures are formed. Residues can be linked, optionally through a linker (e.g., L^{T}) at any suitable positions. For example, a linkage between two residues can connect each residue independently at its N-terminus, C-terminus, a point on the backbone, or a point on a side chain, etc. In some embodiments, two or more side chains of residues, e.g., in compounds of formula I-a, (e.g., R^{a2} or R^{a3} of one amino acid residue with R^{a2} or R^{a3} of another amino acid residue) are optionally take together to form a bridge, e.g., in some embodiments, two cysteine residues form a —S—S— bridge as typically observed in natural proteins. In some embodiments, a formed bridge has the structure of L^{b} wherein L^{b} is L^{a} as described in the present disclosure. In some embodiments, each end of L^{b} independently connects to a backbone atom of a cyclic peptide (e.g., a ring atom of the ring formed by -(Xaa)_{z}- in formula I-a). In some embodiments, L^{b} comprises an R group (e.g., when a methylene unit of L^{b} is replaced with —C(R)₂— or —N(R)—), wherein the R group is taken together with an R group attached to a backbone atom (e.g., R^{a1}, R^{a2}, R^{a3}, etc. if being R) and their intervening atoms to form a ring. In some embodiments, L^{b} connects to a ring, e.g., the ring formed by -(Xaa)_{z}- in formula I-a through a side chain of an amino acid residue (e.g., Xaa in formula I-a). In some embodiments, such a side chain comprises an amino group or a carboxylic acid group. In some embodiments, L^{T} is L^{b} as described herein. In some embodiments, a linkage, e.g., L^{b} or L^{T}, connects a side chain with a N-terminus or a C-terminus of a residue. In some embodiments, a linkage connects a side chain with an amino group of a residue. In some embodiments, a linkage connects a side chain with an alpha-amino group of a residue. In some embodiments, as illustrated herein, a linkage, e.g., L^{b} or L^{T}, is —CH₂—C(O)—. In some embodiments, the —CH₂— is bonded to a side chain, e.g., bound to —S— of a cysteine residue, and the —C(O)— is bonded to an amino group, e.g., an alpha-amino group of a residue. In some embodiments, a linkage, e.g., L^{b} or L^{T}, is optionally substituted —CH₂—S—CH₂—C(O)—NH—, wherein each end is bonded to the alpha-carbon of a residue. In some embodiments, the —NH— is of an alpha-amino group of a residue, e.g., of a N-terminal residue.

In some embodiments,

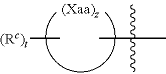

is an antibody binding moiety

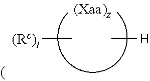

binds to an antibody). In some embodiments,

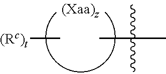

is a universal antibody binding moiety. In some embodiments,

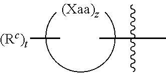

is a universal antibody binding moiety which can bind to antibodies having different Fab regions. In some embodiments,

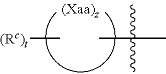

is a universal antibody binding moiety that can bind to a Fc region. In some embodiments, an antibody binding moiety, e.g., a universal antibody binding moiety having the structure of

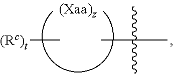

can bind to a Fc region bound to an Fc receptor. In some embodiments, an antibody binding moiety, e.g., of an antibody binding moiety having the structure of

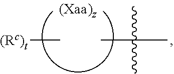

has the structure of

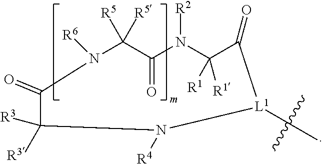

In some embodiments,

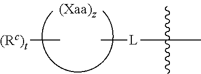

has the structure of

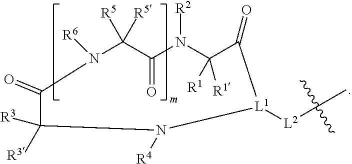

In certain embodiments, the present disclosure provides a compound of formula II:

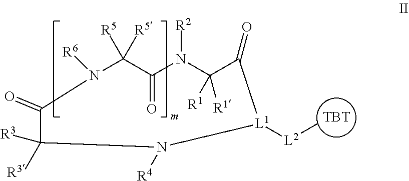

or a pharmaceutically acceptable salt thereof, wherein:
each of R¹, R³ and R⁵ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or:
R¹ and R^{1′} are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur;
R³ and R^{3′} are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur;
an R⁵ group and the R^{5′} group attached to the same carbon atom are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur; or
two R⁵ groups are optionally taken together with their intervening atoms to form a C₁₋₁₀ optionally substituted bivalent straight or branched saturated or unsaturated hydrocarbon chain wherein 1-3 methylene units of the chain are independently and optionally replaced with —S—, —SS—, —N(R)—, —O—, —C(O)—, —OC(O)—, —C(O)O—, —C(O)N(R)—, —N(R)C(O)—, —S(O)—, —S(O)₂—, or -Cy¹-, wherein each -Cy¹- is independently a 5-6 membered heteroarylenyl with 1-4 heteroatoms independently selected from nitrogen, oxygen or sulfur;
each of R^{1′}, R^{3′} and R^{5′} is independently hydrogen or optionally substituted C₁₋₃ aliphatic;
each of R², R⁴ and R⁶ is independently hydrogen, or optionally substituted C₁₋₄ aliphatic, or:
   R² and R¹ are optionally taken together with their intervening atoms to form a 4-8 membered, optionally substituted saturated or partially unsaturated monocyclic heterocyclic ring having 1-2 heteroatoms independently selected from nitrogen, oxygen, or sulfur;
   R⁴ and R³ are optionally taken together with their intervening atoms to form a 4-8 membered optionally substituted saturated or partially unsaturated monocyclic heterocyclic ring having 1-2 heteroatoms independently selected from nitrogen, oxygen, or sulfur; or
   an R⁶ group and its adjacent R⁵ group are optionally taken together with their intervening atoms to form a 4-8 membered optionally substituted saturated or partially unsaturated monocyclic heterocyclic ring having 1-2 heteroatoms independently selected from nitrogen, oxygen, or sulfur;
L¹ is a trivalent linker moiety that connects

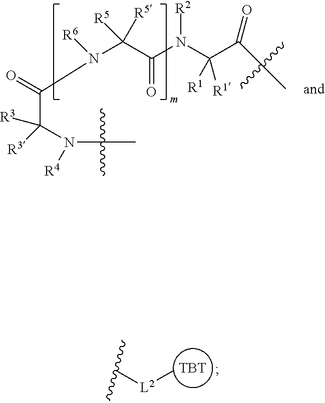

L² is a covalent bond or a C₁₋₃₀ optionally substituted bivalent straight or branched saturated or unsaturated hydrocarbon chain wherein 1-10 methylene units of the chain are independently and optionally replaced with —S—, —N(R)—, —O—, —C(O)—, —OC(O)—, —C(O)O—, —C(O)N(R)—, —N(R)C(O)—, —S(O)—, —S(O)₂—, —(CH₂OCH₂)n-, —(OCH₂CH₂)n-, or -Cy¹-, wherein each -Cy¹- is independently a 5-6 membered heteroarylenyl with 1-4 heteroatoms independently selected from nitrogen, oxygen or sulfur;
TBT is a target binding moiety; and
each of m and n is independently 1, 2, 3, 4, 5, 6, 7, 8, 9, or 10.

In some embodiments, an antibody binding moiety is or comprises a peptide moiety. In some embodiments, the present disclosure provides a compound having the structure of formula I-b:

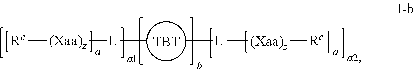

or a salt thereof, wherein:
each Xaa is independently a residue of an amino acid or an amino acid analog;
each z is independently 1-50;
each L is independently a linker moiety;
TBT is a target binding moiety,
each R^{c} is independently -L^{a}-R′;
each of a1 and a2 is independently 0 or 1, wherein at least one of a1 and a2 is not 0;
each of a and b is independently 1-200;
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, a1 is 1. In some embodiments, a2 is 1. In some embodiments, b is 1. In some embodiments, a compound of formula I-b has the structure of

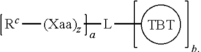

In some embodiments, a compound of formula I-b has the structure of

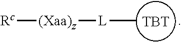

In some embodiments, a compound of formula I-b has the structure of

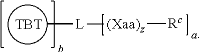

In some embodiments, a compound of formula I-b has the structure of

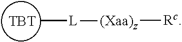

In some embodiments, each residue, e.g., each Xaa in formula I-a, I-b, etc., is independently a residue of amino acid having the structure of formula A-I. In some embodiments, each Xaa independently has the structure of —N(R^{a1})-L^{a1}-C(R^{a2})(R^{a3})-L^{a2}-CO—. In some embodiments, two or more side chains of the amino acid residues, e.g., in compounds of formula I-a, (e.g., R^{a2} or R³ of one amino acid residue with R^{a2} or R^{a3} of another amino acid residue) are optionally take together to form a bridge, e.g., in some embodiments, two cysteine residues form a —S—S— bridge as typically observed in natural proteins. In some embodiments, a formed bridge has the structure of L^{b}, wherein L^{b} is L^{a} as described in the present disclosure. In some embodiments, each end of L^{b} independently connects to a backbone atom of a cyclic peptide (e.g., a ring atom of the ring formed by -(Xaa)_{z}- in formula I-a). In some embodiments, L^{b} comprises an R group (e.g., when a methylene unit of L^{b} is replaced with —C(R)₂— or —N(R)—), wherein the R group is taken together with an R group attached to a backbone atom (e.g., R^{a1}, R^{a2}, R^{a3}, etc. if being R) and their intervening atoms to form a ring. In some embodiments, L^{b} connects to a ring, e.g., the ring formed by -(Xaa)Z— in formula I-b through a side chain of an amino acid residue (e.g., Xaa in formula I-a). In some embodiments, such a side chain comprises an amino group or a carboxylic acid group.

In some embodiments, R^{c}-(Xaa)z- is an antibody binding moiety (R-(Xaa)z-H binds to an antibody). In some embodiments, R^{c}-(Xaa)z- is a universal antibody binding moiety. In some embodiments, R^{c}-(Xaa)z- is a universal antibody binding moiety which can bind to antibodies having different Fab regions. In some embodiments, R^{c}-(Xaa)z- is a universal antibody binding moiety that can bind to a Fc region. In some embodiments, an antibody binding moiety, e.g., a universal antibody binding moiety having the structure of R^{c}-(Xaa)z-, can bind to a Fc region which binds to an Fc receptor. In some embodiments, R^{c}-(Xaa)z- has the structure of

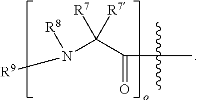

In some embodiments, R^{c}-(Xaa)z-L- has the structure of

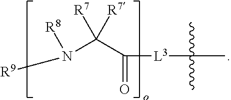

In certain embodiments, the present disclosure provides a compound of formula III:

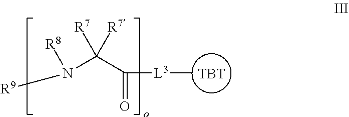

or a pharmaceutically acceptable salt thereof, wherein:
each of R⁷ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or:
   an R⁷ group and the R^{7′} group attached to the same carbon atom are optionally taken together with their intervening carbon atom to form a 3-8 membered optionally substituted saturated or partially unsaturated spirocyclic carbocyclic ring or a 3-8 membered optionally substituted saturated or partially unsaturated spirocyclic heterocyclic ring having 1-2 heteroatoms independently selected from nitrogen, oxygen, or sulfur;
each of R^{7′} is independently hydrogen or optionally substituted C₁₋₃ aliphatic;
each of R⁸ is independently hydrogen, or optionally substituted C₁₋₄ aliphatic, or:
   an R⁸ group and its adjacent R⁷ group are optionally taken together with their intervening atoms to form a 4-8 membered optionally substituted saturated or partially unsaturated monocyclic heterocyclic ring having 1-2 heteroatoms independently selected from nitrogen, oxygen, or sulfur;
R⁹ is hydrogen, optionally substituted C₁₋₃ aliphatic, or —C(O)— (optionally substituted C₁₋₃ aliphatic);
L³ is a bivalent linker moiety that connects

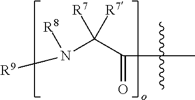

with TBT;
TBT is a target binding moiety; and
o is 1, 2, 3, 4, 5, 6, 7, 8, 9, or 10.

In some embodiments, an amino acid has the structure of formula A-I:

NH(R^{a1})-L^{a1}-C(R^{a2})(R^{a3})-L^{a2}-COOH, A-I

or a salt thereof, wherein:
each of R^{a1}, R^{a2}, R^{a3} is independently -L^{a}-R′;
each of L^{a1} and L^{a2} is independently L^{a};
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, a residue has the structure of —N(R^{a1})-L^{a1}-C(R^{a2})(R^{a3})-L^{a2}-COO— or a salt form thereof.

In some embodiments, an amino acid analog is a compound in which the amino group and/or carboxylic acid group are independently replaced with an optionally substituted aliphatic or heteroaliphatic moiety. As those skilled in the art will appreciate, many amino acid analogs, which mimics structures, properties and/or functions of amino acids, are described in the art and can be utilized in accordance with the present disclosure, e.g., in various moieties. In some embodiments, one or more peptide groups are optionally and independently replaced with non-peptide groups. In some embodiments, an amino acid moiety in a polypeptide or peptide is replaced with an amino acid analog moiety.

### 2. Definitions

Compounds of the present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included. Unless otherwise specified, compounds described herein may be provided and/or utilized in a salt form, particularly a pharmaceutically acceptable salt form.

Agent: In general, the term “agent”, as used herein, may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety. In some embodiments, an agent is a compound.

Aliphatic: As used herein, “aliphatic” means a straight-chain (i.e., unbranched) or branched, substituted or unsubstituted hydrocarbon chain that is completely saturated or that contains one or more units of unsaturation, or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, an alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Alkynyl: As used herein, the term “alkynyl” refers to an aliphatic group, as defined herein, having one or more triple bonds.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)- an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH₂ domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered such glycosylation. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present disclosure is in a format selected from, but not limited to, intact IgA, IgG, IgE or IgM antibodies; bi- or multi-specific antibodies (e.g., Zybodies®, additional bi- or multi-specific antibodies described in Ulrich Brinkmann & Roland E. Kontermann (2017) The making of bispecific antibodies, mAbs, 9:2, 182-212, doi: 10.1080/19420862.2016.1268307, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; KALBITOR®s; CovX-Bodies; and CrossMabs. In some embodiments, antibodies may have enhanced Fc domains. In some embodiments, antibodies may comprise one or more unnatural amino acid residues. In some embodiments, an antibody may lack a covalent modification (e.g., attachment of a glycan) that it would have if produced naturally. In some embodiments, an antibody is an afucosylated antibody. In some embodiments, an antibody is conjugated with another entity. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.]).

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, an aryl group is a biaryl group. The term “aryl” may be used interchangeably with the term “aryl ring.” In certain embodiments of the present disclosure, “aryl” refers to an aromatic ring system which includes, but is not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

Cycloaliphatic: The term “cycloaliphatic,” “carbocycle,” “carbocyclyl,” “carbocyclic radical,” and “carbocyclic ring,” are used interchangeably, and as used herein, refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, a cycloaliphatic group has 3-6 carbons. In some embodiments, a cycloaliphatic group is saturated and is cycloalkyl. The term “cycloaliphatic” may also include aliphatic rings that are fused to one or more aromatic or nonaromatic rings, such as decahydronaphthyl or tetrahydronaphthyl. In some embodiments, a cycloaliphatic group is bicyclic. In some embodiments, a cycloaliphatic group is tricyclic. In some embodiments, a cycloaliphatic group is polycyclic. In some embodiments, “cycloaliphatic” refers to C₃-C₆ monocyclic hydrocarbon, or C₈-C₁₀ bicyclic or polycyclic hydrocarbon, that is completely saturated or that contains one or more units of unsaturation, but which is not aromatic, that has a single point of attachment to the rest of the molecule, or a C₉-C₁₆ polycyclic hydrocarbon that is completely saturated or that contains one or more units of unsaturation, but which is not aromatic, that has a single point of attachment to the rest of the molecule.

Dosing regimen: As used herein, a “dosing regimen” or “therapeutic regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount.

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In some embodiments, one or more units selected from C, CH, CH₂, and CH₃ are independently replaced by one or more heteroatoms (including oxidized and/or substituted forms thereof). In some embodiments, a heteroaliphatic group is heteroalkyl. In some embodiments, a heteroaliphatic group is heteroalkenyl.

Heteroalkyl: The term “heteroalkyl”, as used herein, is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-”, as used herein, used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic or polycyclic), in some embodiments 5, 6, 9, or 10 ring atoms. In some embodiments, a heteroaryl group has 6, 10, or 14π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom”, as used herein, means an atom that is not carbon or hydrogen. In some embodiments, a heteroatom is boron, oxygen, sulfur, nitrogen, phosphorus, or silicon (including various forms of such atoms, such as oxidized forms (e.g., of nitrogen, sulfur, phosphorus, or silicon), quaternized form of a basic nitrogen or a substitutable nitrogen of a heterocyclic ring (for example, N as in 3,4-dihydro-2H-pyrrolyl), NH (as in pyrrolidinyl) or NW (as in N-substituted pyrrolidinyl) etc.). In some embodiments, a heteroatom is oxygen, sulfur or nitrogen.

Heterocycle: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Lower alkyl: The term “lower alkyl” refers to a C₁₋₄ straight or branched alkyl group. Example lower alkyl groups are methyl, ethyl, propyl, isopropyl, butyl, isobutyl, and tert-butyl.

Lower haloalkyl: The term “lower haloalkyl” refers to a C₁₋₄ straight or branched alkyl group that is substituted with one or more halogen atoms.

Optionally Substituted: As described herein, compounds of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In some embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Certain substituents are described below.

Suitable monovalent substituents on a substitutable atom, e.g., a suitable carbon atom, are independently halogen; —(CH₂)₀₋₄R^{◯}; —(CH₂)₀₋₄OR^{◯}; —O(CH₂)₀₋₄R^{◯}, —O—(CH₂)₀₋₄C(O)OR^{◯}; —(CH₂)₀₋₄CH(OR^{◯})₂; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —(CH₂)₀₋₄O(CH₂)₀₋₁-pyridyl which may be substituted with R^{◯}; —NO₂; —CN; —N₃; —(CH₂)₀₋₄N(R^{◯})₂; —(CH₂)₀₋₄N(R^{◯})C(O)R^{◯}; —N(R^{◯})C(S)R^{◯}; —(CH₂)₀₋₄N(R^{◯})C(O)NR^{◯}₂; —N(R^{◯})C(S)NR^{◯}₂; —(CH₂)₀₋₄N(R^{◯})C(O)OR^{◯}; —N(R^{◯})N(R^{◯})C(O)R^{◯}; —N(R^{◯})N(R^{◯})C(O)NR^{◯}₂; —N(R^{◯})N(R^{◯})C(O)OR^{◯}; —(CH₂)₀₋₄C(O)R^{◯}; —C(S)R^{◯}; —(CH₂)₀₋₄C(O)OR^{◯}; —(CH₂)₀₋₄C(O)SR^{◯}; —(CH₂)₀₋₄C(O)OSiR^{◯}₃; —(CH₂)₀₋₄OC(O)R^{◯}; —OC(O)(CH₂)₀₋₄SR^{◯}, —SC(S)SR^{◯}; —(CH₂)₀₋₄SC(O)R^{◯}; —(CH₂)₀₋₄C(O)NR⁰²; —C(S)NR^{◯}₂; —C(S)SR^{◯}; —(CH₂)₀₋₄OC(O)NR^{◯}₂; —C(O)N(OR^{◯})R^{◯}; —C(O)C(O)R^{◯}; —C(O)CH₂C(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; —(CH₂)₀₋₄S(O)R^{◯}; —N(R^{◯})S(O)₂NR^{◯}₂; —N(R^{◯})S(O)₂R^{◯}; —N(OR^{◯})R^{◯}; —C(NH)NR^{◯}₂; —Si(R^{◯})₃; —OSi(R^{◯})₃; —B(R^{◯})₂; —OB(R^{◯})₂; —OB(OR^{◯})₂; —P(R^{◯})₂; —P(OR^{◯})₂; —P(R^{◯})(OR^{◯}); —OP(R^{◯})₂; —OP(OR^{◯})₂; —OP(R^{◯})(OR^{◯}); —P(O)(R^{◯})₂; —P(O)(OR^{◯})₂; —OP(O)(R^{◯})₂; —OP(O)(OR^{◯})₂; —OP(O)(OR^{◯})(SR^{◯}); —SP(O)(R^{◯})₂; —SP(O)(OR^{◯})₂; —N(R^{◯})P(O)(R^{◯})₂; —N(R^{◯})P(O)(OR^{◯})₂; —P(R^{◯})₂[B(R^{◯})₃]; —P(OR^{◯})₂[B(R^{◯})₃]; —OP(R^{◯})₂[B(R^{◯})₃]; —OP(OR^{◯})₂[B(R^{◯})₃]; —(C₁₋₄ straight or branched alkylene)O—N(R^{◯})₂; or —(C₁₋₄ straight or branched alkylene)C(O)O—N(R^{◯})₂, wherein each R^{◯} may be substituted as defined herein and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH2)₀₋₁(C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{◯}, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{◯} (or the ring formed by taking two independent occurrences of R^{◯} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR′, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of R^{◯} include ═O and ═S.

Suitable divalent substituents, e.g., on a suitable carbon atom, are independently the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5-6-membered saturated, partially unsaturated, and aryl ring having 0-4 heteroatoms independently selected from nitrogen, oxygen, and sulfur.

Suitable substituents on the aliphatic group of R* are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

In some embodiments, suitable substituents on a substitutable nitrogen are independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Peptide: The term “peptide” as used herein refers to a polypeptide that is typically relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids. In some embodiments, a peptide has a length of about 5-100, e.g., about 5, 10, 15, 20, 30, 40, 50, 60, 70, 80, 90, or 100 amino acids.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a compound comprises one or more acidic groups and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a compound comprises more than one acid groups. In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations.

Polypeptide: As used herein refers to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplar polypeptides within the class whose amino acid sequences and/or functions are known; in some embodiments, such exemplar polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. June 2012, the entirety of Chapter 2 is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S— benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifiuoroacetyl, pivaloyl, 9-fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-y1 (MOX). In some embodiments, each of the hydroxyl protecting groups is, independently selected from acetyl, benzyl, t-butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group. In some embodiments, a phosphorous linkage protecting group is a group attached to the phosphorous linkage (e.g., an internucleotidic linkage) throughout oligonucleotide synthesis. In some embodiments, a protecting group is attached to a sulfur atom of an phosphorothioate group. In some embodiments, a protecting group is attached to an oxygen atom of an internucleotide phosphorothioate linkage. In some embodiments, a protecting group is attached to an oxygen atom of the internucleotide phosphate linkage. In some embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl, N-methyl)aminoethyl, or 4-[N-methyl-N-(2,2,2-trifluoroacetyl)amino]butyl.

Subject: As used herein, the term “subject” refers to any organism to which an agent, a compound or a composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In some embodiments, a subject is a human. In some embodiments, a subject may be suffering from and/or susceptible to a disease, disorder and/or condition.

Substantially: As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition is predisposed to have that disease, disorder and/or condition. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the term “therapeutic agent” in general refers to any agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, pre-existing clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, a therapeutic agent is a compound described herein.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unit dose: The expression “unit dose” as used herein refers to an amount administered as a single dose and/or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included as described infra. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

Unsaturated: The term “unsaturated,” as used herein, means that a moiety has one or more units of unsaturation.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the present disclosure. Unless otherwise stated, all tautomeric forms of the compounds are within the scope of the present disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of the present disclosure. Such compounds are useful, for example, as analytical tools, as probes in biological assays, or as therapeutic agents in accordance with the present disclosure. Unless otherwise stated, salts/salt forms of compounds, agents, moieties, etc., are included.

### 3. Description of Certain Exemplified Embodiments

In some embodiments, the present disclosure provide an agent comprising a target binding moiety as described herein.

In some embodiments, agents bind to spike proteins (e.g., at S1 and/or S2 domains), blocking viruses from binding ACE2 receptor and infecting human cells. In some embodiments, agents recruit immune cells to attack, inhibit, kill or remove viruses and/or virus-infected cells (e.g., macrophages, NK cells, etc.), in some embodiments, through interactions with FcγRII-III receptors. In some embodiments, agents recruit dendritic cells, and in some embodiments, induce, promote, encourage, enhance, or trigger an immune system to adapt to proteins. In some embodiments, long-term immunity is provided. In some embodiments, immune memory cells (e.g., T-cells and/or B-cells) are generated to instill long-term immunity. In some embodiments, agents recruit IgG1 and IgG2 (e.g., those in human blood stream). In some embodiments, agents recruit IgG1, IgG2 and IgG4 (e.g., those in human blood stream). In some embodiments, agents recruit IgG1, IgG2, IgG3 and IgG4 (e.g., those in human blood stream). In some embodiments, agents comprise IgG1 and IgG2 (e.g., in antibody moieties). In some embodiments, agents comprise IgG1, IgG2 and IgG4. In some embodiments, agents comprise IgG1, IgG2, IgG3 and IgG4.

In some embodiments, a provide agent or a target binding moiety, e.g., of or comprising -(Xaa)y-, is selective for SARS-CoV-2 or a protein or a fragment thereof. In some embodiments, a provided agent or target binding moiety can target two or more types of virus, e.g., through interactions with proteins having similar sequences and/or structures. In some embodiments, provided agents and/or compositions thereof can effectively target two or more or all coronaviruses. In some embodiments, provided agents and/or target binding moieties can effectively target two or more or all coronaviruses that infect humans. In some embodiments, provided agents and/or compositions thereof can effectively target two or more or all coronaviruses that share similar sequences/structures of spike proteins or fragments thereof (e.g., portions outside of viruses, portions interacting with human receptors, portions involved in infection humans, etc.). In some embodiments, provided agents and/or target binding moieties target SARS-CoV. In some embodiments, provided agents and/or target binding moieties target MERS-CoV. In some embodiments, provided agents and/or target binding moieties can target SARS-CoV, SARS-CoV-2 and/or MERS-CoV. In some embodiments, provided agents and/or target binding moieties can target SARS-CoV and SARS-CoV-2. In some embodiments, provided agents and/or target binding moieties can target SARS-CoV, SARS-CoV-2 and MERS-CoV. Among other things, the present disclosure provides technologies for inducing, promoting, encouraging, enhancing, triggering, or generating an immune response toward one or two or all of SARS-CoV, SARS-CoV-2 and MERS-CoV. In some embodiments, an immune response is or comprises ADCC, ADCP and/or long-term immunity as described herein. In some embodiments, the present disclosure provides technologies for inhibiting, killing or removing SARS-CoV, SARS-CoV-2 and/or MERS-CoV viruses. In some embodiments, the present disclosure provides technologies for inhibiting, killing or removing cells infected by SARS-CoV, SARS-CoV-2 and/or MERS-CoV viruses. In some embodiments, the present disclosure provides technologies for preventing or treating conditions, disorders or diseases associated with SARS-CoV, SARS-CoV-2 and/or MERS-CoV. In some embodiments, the present disclosure provides technologies for preventing or treating conditions, disorders or diseases associated with SARS-CoV (e.g., severe acute respiratory syndrome). In some embodiments, the present disclosure provides technologies for preventing or treating conditions, disorders or diseases associated with SARS-CoV-2 (e.g., COVID-19). In some embodiments, the present disclosure provides technologies for preventing or treating conditions, disorders or diseases associated with MERS-CoV (e.g., Middle East respiratory syndrome). In some embodiments, the present disclosure provides a method for disrupting, reducing or preventing an infection by SARS-CoV, SARS-CoV-2 and/or MERS-CoV viruses. In some embodiments, provided technologies are useful for inducing, promoting, encouraging, enhancing, triggering, or generating an immune response toward, and/or for inhibiting, killing or removing, and/or for inhibiting, killing or removing cells infected by, and/or for preventing or treating conditions, disorders or diseases associated with, and/or for disrupting, reducing or preventing an infection by, SARS-CoV, SARS-CoV-2 and MERS-CoV viruses. In some embodiments, provided technologies comprise contacting viruses with an effective amount of an agent or composition as described herein. In some embodiments, provided technologies comprise administering to a subject susceptible to or suffering from viral infections and/or conditions, disorders or diseases associated with viral infections an effective amount of an agent or composition as described herein.

In some embodiments, an agent binds to its target or a portion thereof with a K_{D} that is about or no more than about 1, 2, 3, 4, 5, 6, 7, 8, 9, or 10 uM, or is about or no more than about 500, 200, 100, 50, 10, 1, 0.5, 0.2 or 0.1 nM. In some embodiments, an agent binds to a SARS-CoV-2 spike receptor binding domain with K_{D} of no more than 10,000, 5,000, 2,000, 1,000, 500, 200, 100, 50, 10, 1, 0.5, 0.2 or 0.1 nM. Various technologies can be utilized to measure binding K_{D} in accordance with the present disclosure; certain technologies are described in the Examples.

### Target Binding Moieties

In some embodiments, the present disclosure provides agents that can bind to a SARS-CoV-2 virus or a cell infected thereby. Among other things, agents of the present disclosure comprise target binding moieties that can bind to a SARS-CoV-2 spike protein or a fragment thereof. In some embodiments, the present disclosure provides agents that can bind to a SARS-CoV-2 spike protein or a fragment thereof. In some embodiments, target binding moieties are or comprise peptide moieties.

In some embodiments, a target binding moiety is or comprises a peptide agent. In some embodiments, a target binding moiety is a peptide moiety. In some embodiments, a peptide moiety can either be linier or cyclic. In some embodiments, a target binding moiety is or comprises a peptide moiety comprising a cyclic structure.

In some embodiments, a provided agent has the structure of R^{CN}-(Xaa)y-R^{CC} or a salt thereof. In some embodiments, a provided target binding moiety is a moiety of R^{CN}-(Xaa)y-R^{CC} or a salt thereof (e.g., removing one or more —H to form a monovalent, bivalent or polyvalent moiety). In some embodiments, a target binding moiety is or comprises -(Xaa)y- as described herein. In some embodiments, as described herein a target binding moiety may be connected to the rest of the molecule, an antibody moiety, or an antibody binding moiety through a N-terminus, C-terminus and/or a middle residue (e.g. through a side chain thereof). In some embodiments, a target binding moiety comprises -(Xaa)y-.

In some embodiments, a target binding moiety, or -(Xaa)y-, is or comprises a sequence that is or shares at least about 50%, 55%, 60%, 65%, 70%, 75%, 80%, 85% 90%, 91%, 92%, 93%, 94%, 95%, 96%, 97%, 98% or 99% identity with a sequence selected from: DEDLEELERLYRKAEEVAKEAKDASRRGDDERAKEQMERAMRLFDQVFELAQELQEKQTDGN RQKATHLDKAVKEAADELYQRVR (SEQ ID NO: 1), ELEEQVMHVLDQVSELAHELLHKLTGEELERAAYFNWWATEMMLELIKSDDEREIREIEEEARR ILEHLEELARK (SEQ ID NO: 2), DKEWILQKIYEIMRLLDELGHAEASMRVSDLIYEFMKKGDERLLEEAERLLEEVER (SEQ ID NO: 3), DKEEILNKIYEIMRLLDELGNAEASMRVSDLILEFMKKGDERLLEEAERLLEEVER (SEQ ID NO: 4), SDDEDSVRYLLYMAELRYEQGNPEKAKKILEMAEFIAKRNNNEELERLVREVKKRL (SEQ ID NO: 5), NDDELHMLMTDLVYEALHFAKDEEIKKRVFQLFELADKAYKNNDRQKLEKVVEELKELLERLL S (SEQ ID NO: 6), QREKRLKQLEMLLEYAIERNDPYLMFDVAVEMLRLAEENNDERIIERAKRILEEYE (SEQ ID NO: 7), SLEELKEQVKELKKELSPEMRRLIEEALRFLEEGNPAMAMMVLSDLVYQLGDPRVIDLYMLVTK T (SEQ ID NO: 8), DREQRLVRFLVRLASKFNLSPEQILQLFEVLEELLERGVSEEEIRKQLEEVAKELG (SEQ ID NO: 9), DDDIRYLIYMAKLRLEQGNPEEAEKVLEMARFLAERLGMEELLKEVRELLRKIEELR (SEQ ID NO: 10), and PIIELLREAKEKNDEFAISDALYLVNELLQRTGDPRLEEVLYLIWRALKEKDPRLLDRAIELFER (SEQ ID NO: 11).

In some embodiments, an identity is 50% or more. In some embodiments, an identity is 55% or more. In some embodiments, an identity is 60% or more. In some embodiments, an identity is 65% or more. In some embodiments, an identity is 70% or more. In some embodiments, an identity is 75% or more. In some embodiments, an identity is 80% or more. In some embodiments, an identity is 85% or more. In some embodiments, an identity is 90% or more. In some embodiments, an identity is 91% or more. In some embodiments, an identity is 92% or more. In some embodiments, an identity is 93% or more. In some embodiments, an identity is 94% or more. In some embodiments, an identity is 95% or more. In some embodiments, an identity is 96% or more. In some embodiments, an identity is 97% or more. In some embodiments, an identity is 98% or more. In some embodiments, an identity is 99% or more. In some embodiments, a target binding moiety, or -(Xaa)y-, is or comprises a sequence that is selected from SEQ ID NOs 1-11. In some embodiments, a target binding moiety, or -(Xaa)y-, is a peptide moiety whose sequence comprises a sequence selected from SEQ ID NOs 1-11. In some embodiments, a target binding moiety, or -(Xaa)y-, is a peptide moiety whose sequence is a sequence selected from SEQ ID NOs 1-11. In some embodiments, a sequence is SEQ ID NO: 1. In some embodiments, a sequence is SEQ ID NO: 2. In some embodiments, a sequence is SEQ ID NO: 3. In some embodiments, a sequence is SEQ ID NO: 4. In some embodiments, a sequence is SEQ ID NO: 5. In some embodiments, a sequence is SEQ ID NO: 6. In some embodiments, a sequence is SEQ ID NO: 7. In some embodiments, a sequence is SEQ ID NO: 8. In some embodiments, a sequence is SEQ ID NO: 9. In some embodiments, a sequence is SEQ ID NO: 10. In some embodiments, a sequence is SEQ ID NO: 11.

In some embodiments, a target binding moiety, or -(Xaa)y-, is or comprises a sequence that is selected from SEQ ID NOs 1-11 with 0-10 deletions, 0-10 additions and 0-10 replacements. In some embodiments, there are one or more deletions, additions and/or replacements. In some embodiments, there is one or more deletions. In some embodiments, there is one or more additions. In some embodiments, there is one or more replacements. In some embodiments, the total number of deletions, additions and replacements, if any, is no more than 1, 2, 3, 4, 5, 6, 7, 8, 9, 10, 11, 12, 13, 14, 15, 16, 17, 18, 19, 20, 21, 22, 23, 24, or 25. In some embodiments, it is no more than 20. In some embodiments, it is no more than 19. In some embodiments, it is no more than 18. In some embodiments, it is no more than 17. In some embodiments, it is no more than 16. In some embodiments, it is no more than 15. In some embodiments, it is no more than 14. In some embodiments, it is no more than 13. In some embodiments, it is no more than 12. In some embodiments, it is no more than 11. In some embodiments, it is no more than 10. In some embodiments, it is no more than 9. In some embodiments, it is no more than 8. In some embodiments, it is no more than 7. In some embodiments, it is no more than 6. In some embodiments, it is no more than 5. In some embodiments, it is no more than 4. In some embodiments, it is no more than 3. In some embodiments, it is no more than 2. In some embodiments, it is no more than 1.

In some embodiments, R^{CN} is R—C(O)—. In some embodiments, R is optionally substituted C₁₋₆ aliphatic. In some embodiments, R is methyl.

In some embodiments, R^{CC} is —N(R′)₂. In some embodiments, R^{CC} is —N(R)₂. In some embodiments, R^{CC} is —NH₂.

In some embodiments, a peptide unit, e.g., a target binding moiety, comprises a functional group in an amino acid residue that can react with a functional group of another amino acid residue. In some embodiments, a peptide unit comprises an amino acid residue with a side chain which comprises a functional group that can react with another functional group of the side chain of another amino acid residue to form a linkage. In some embodiments, one functional group of one amino acid residue is connected to a functional group of another amino acid residue to form a linkage (or bridge). Linkages are bonded to backbone atoms of peptide units and comprise no backbone atoms. In some embodiments, a peptide unit comprises a linkage formed by two side chains of non-neighboring amino acid residues. In some embodiments, a linkage is bonded to two backbone atoms of two non-neighboring amino acid residues. In some embodiments, both backbone atoms bonded to a linkage are carbon atoms. In some embodiments, a linkage has the structure of L^{b}, wherein L^{b} is L^{a} as described in the present disclosure, wherein L^{a} is not a covalent bond. In some embodiments, L^{a} comprises -Cy-. In some embodiments, L^{a} comprises -Cy-, wherein -Cy- is optionally substituted heteroaryl. In some embodiments, -Cy- is

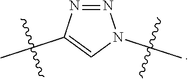 [SMILES: *c1cn(*)nn1]

In some embodiments, L^{a} is

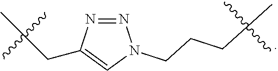 [SMILES: *CCCn1cc(C*)nn1]

In some embodiments, such an L^{a} can be formed by a —N₃ group of the side chain of one amino acid residue, and the -≡- of the side chain of another amino acid residue. In some embodiments, a linkage is formed through connection of two thiol groups, e.g., of two cysteine residues. In some embodiments, L^{a} comprises —S—S—. In some embodiments, L^{a} is —CH₂—S—S—CH₂—. In some embodiments, a linkage is formed through connection of an amino group (e.g., —NH₂ in the side chain of a lysine residue) and a carboxylic acid group (e.g., —COOH in the side chain of an aspartic acid or glutamic acid residue). In some embodiments, L^{a} comprises —C(O)—N(R′)—. In some embodiments, L^{a} comprise —C(O)—NH—. In some embodiments, L^{a} is —CH₂CONH—(CH₂)₃—. In some embodiments, L^{a} comprises —C(O)—N(R′)—, wherein R′ is R, and is taken together with an R group on the peptide backbone to form a ring (e.g., in A-34). In some embodiments, L^{a} is —(CH₂)₂—N(R′)—CO—(CH₂)₂—. In some embodiments, -Cy- is optionally substituted phenylene. In some embodiments, -Cy- is optionally substituted 1,2-phenylene. In some embodiments, L^{a} is

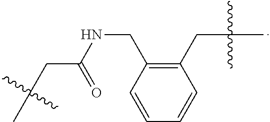 [SMILES: *CC(=O)NCc1ccccc1C*]

In some embodiments, L^{a} is

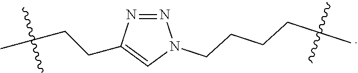 [SMILES: *CCCCn1cc(CC*)nn1]

In some embodiments, L^{a} is optionally substituted bivalent C₂₋₂₀ bivalent aliphatic. In some embodiments, L^{a} is optionally substituted —(CH₂)₉—CH═CH—(CH₂)₉—. In some embodiments, L^{a} is —(CH₂)₃—CH═CH—(CH₂)₃—.

In some embodiments, two amino acid residues bonded to a linkage are separated by 1, 2, 3, 4, 5, 6, 7, 8, 9, 10, 11, 12, 13, 14, 15, or more than 15 amino acid residues between them (excluding the two amino acid residues bonded to the linkage). In some embodiments, the number is 1. In some embodiments, the number is 2. In some embodiments, the number is 3. In some embodiments, the number is 4. In some embodiments, the number is 5. In some embodiments, the number is 6. In some embodiments, the number is 7. In some embodiments, the number is 8. In some embodiments, the number is 9. In some embodiments, the number is 10. In some embodiments, the number is 11. In some embodiments, the number is 12. In some embodiments, the number is 13. In some embodiments, the number is 14. In some embodiments, the number is 15.

In some embodiments, a target binding moiety comprises a peptide unit, and an antibody binding moiety is connected to a backbone atom of the peptide unit optionally via a linker. In some embodiments, a target binding moiety comprises a peptide unit, and an antibody binding moiety is connected to an atom of a side chain, e.g., through an atom or group in the side chain, of an amino acid residue of the peptide unit optionally via a linker. For example, in some embodiments, an antibody binding moiety is connected through a —SH, —OH, —COOH, or —NH₂ of a side chain.

In some embodiments, a target binding moiety binds to its target or a portion thereof with a K_{D} that is about or no more than about 1, 2, 3, 4, 5, 6, 7, 8, 9, or 10 uM, or is about or no more than about 500, 200, 100, 50, 10, 1, 0.5, 0.2 or 0.1 nM. In some embodiments, an agent binds to its target or a portion thereof with a K_{D} that is about or no more than about 1, 2, 3, 4, 5, 6, 7, 8, 9, or 10 uM, or is about or no more than about 500, 200, 100, 50, 10, 1, 0.5, 0.2 or 0.1 nM. In some embodiments, a target binding moiety binds to a SARS-CoV-2 spike protein with K_{D} of no more than 10,000, 5,000, 2,000, 1,000, 500, 200, 100, 50, 10, 5, 2, 1, 0.5, 0.2 or 0.1 nM. In some embodiments, an agent binds to a SARS-CoV-2 spike protein with K_{D} of no more than 10,000, 5,000, 2,000, 1,000, 500, 200, 100, 50, 10, 5, 2, 1, 0.5, 0.2 or 0.1 nM. In some embodiments, a target binding moiety binds to a SARS-CoV-2 spike receptor binding domain with K_{D} of no more than 10,000, 5,000, 2,000, 1,000, 500, 200, 100, 50, 10, 5, 2, 1, 0.5, 0.2 or 0.1 nM. In some embodiments, an agent binds to a SARS-CoV-2 spike receptor binding domain with K_{D} of no more than 10,000, 5,000, 2,000, 1,000, 500, 200, 100, 50, 10, 5, 2, 1, 0.5, 0.2 or 0.1 nM. Various technologies can be utilized to measure binding K_{D} in accordance with the present disclosure; certain technologies are described in the Examples. In some embodiments, K_{D} is assessed using an ELISA technology. In some embodiments, K_{D} is assessed using an Octer technology. In some embodiments, a target binding moiety targets SARS-CoV-2. In some embodiments, a target binding moiety binds to a protein of a SARS-CoV-2 virus. In some embodiments, a target binding moiety binds to a spike protein of a SARS-CoV-2 virus. In some embodiments, a target binding moiety binds to a spike protein or a fragment thereof of a SARS-CoV-2 virus expressed by an infected cell. In some embodiments, a target binding moiety binds to a SARS-CoV-2 spike receptor binding domain. In some embodiments, a target binding moiety binds to a SARS-CoV-2 spike receptor binding domain with K_{D} of no more than about 10000, 5000, 2000, 1000, 500, 200, 100, 50, 10, 5, 2, 1, 0.5, 0.2 or 0.1 nM. In some embodiments, an agent targets SARS-CoV-2. In some embodiments, an agent binds to a protein of a SARS-CoV-2 virus. In some embodiments, an agent binds to a spike protein of a SARS-CoV-2 virus. In some embodiments, an agent binds to a spike protein or a fragment thereof of a SARS-CoV-2 virus expressed by an infected cell. In some embodiments, an agent binds to a SARS-CoV-2 spike receptor binding domain. In some embodiments, an agent binds to a SARS-CoV-2 spike receptor binding domain with K_{D} of no more than about 10000, 5000, 2000, 1000, 500, 200, 100, 50, 10, 5, 2, 1, 0.5, 0.2 or 0.1 nM. In some embodiments, K_{D} is no more than 2000 nM. In some embodiments, K_{D} is no more than about 1000 nM. In some embodiments, K_{D} is no more than about 500 nM. In some embodiments, K_{D} is no more than about 200 nM. In some embodiments, K_{D} is no more than about 100 nM. In some embodiments, K_{D} is no more than about 50 nM. In some embodiments, K_{D} is no more than about 20 nM. In some embodiments, K_{D} is no more than about 10 nM. In some embodiments, K_{D} is no more than about 5 nM. In some embodiments, K_{D} is no more than about 2 nM. In some embodiments, K_{D} is no more than about 1 nM. In some embodiments, K_{D} is no more than about 0.5 nM. In some embodiments, K_{D} is no more than about 0.2 nM. In some embodiments, K_{D} is no more than about 0.1 nM. In some embodiments, K_{D} is for a spike protein of SARS-CoV-2. In some embodiments, K_{D} is for a RBD of a spike protein of SARS-CoV-2. In some embodiments, K_{D} is for a monomer RBD or spike protein. In some embodiments, K_{D} is for a trimer RBD or spike protein. In some embodiments, a target binding moiety competes with binding of human angiotensin-converting enzyme 2 (ACE2) receptor. In some embodiments, an agent competes with binding of human angiotensin-converting enzyme 2 (ACE2) receptor.

In some embodiments, an agent disrupts or reduces the interaction of a SARS-CoV-2 virus with a mammalian cell. In some embodiments, an agent disrupts or reduces an infection of SARS-CoV-2 of a mammalian cell. In some embodiments, an agent inhibits, kills or removes a SARS-CoV-2 virus. In some embodiments, an agent inhibits, kills or removes a cell infected by SARS-CoV-2. In some embodiments, an agent inhibits, kills or removes a cell expressing a spike protein or a fragment thereof of a SARS-CoV-2 virus. In some embodiments, an agent inhibits, kills or removes a SARS-CoV-2 virus. In some embodiments, an agent neutralizes a SARS-CoV-2 virus. In some embodiments, an agent provides long term immunity. In some embodiments, an agent provides memory T and/or B cells against SARS-CoV-2

### Mates

In some embodiments, the present disclosure provides an agent comprising:
an antibody moiety,
a target binding moiety, and
optionally a linker moiety linking an antibody moiety and a target binding moiety.

Such an agent may be referred to as a MATE agent or MATE. In some embodiments, an agent comprises an antibody moiety, a target binding moiety, and a linker moiety linking an antibody moiety and a target binding moiety.

In some embodiments, an agent has the structure of formula M-I:

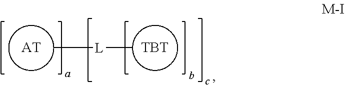

or a pharmaceutically acceptable salt thereof, wherein:
each of a, b and c is independently 1-200;
each AT is independently an antibody moiety;
L is a linker moiety; and
each TBT is independently a target binding moiety.

In some embodiments, an agent has the structure of formula M-II:

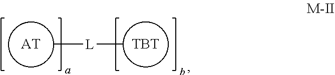

or a pharmaceutically acceptable salt thereof, wherein:
each of a and b is independently 1-200;
each AT is independently an antibody moiety;
L is a linker moiety; and
each TBT is independently a target binding moiety.

In some embodiments, an agent comprises one and no more than one antibody moiety. In some embodiments, one or no more than one antibody moiety is bound to a linker moiety. In some embodiments, a is 1. In some embodiments, an agent comprises two or more antibody moieties. In some embodiments, two or more antibody moieties are bound to a single linker moiety. In some embodiments, a is 2 or more. In some embodiments, one and no more than one target binding moiety is bonded to a linker moiety. In some embodiments, b is 1. In some embodiments, two or more target binding moiety is bonded to a single linker moiety. In some embodiments, b is 2 or more. In some embodiments, an agent comprises one and no more than one target binding moiety. In some embodiments, c is 1. In some embodiments, b is 1 and c is 1. In some embodiments, a is 1, b is 1 and c is 1. In some embodiments, an agent comprises two or more target binding moieties. In some embodiments, b is 2 or more and c is 1. In some embodiments, b is 2 or more and c is 2 or more. In some embodiments, b is 1 and c is 2 or more.

In some embodiments, c is 1-20, e.g., 1, 2, 3, 4, 5, 6, 7, 8, 9, 10, 11, 12, 13, 14, 15, 16, 17, 18, 19, or 20. In some embodiments, c is 1-15. In some embodiments, c is 1-10. In some embodiments, c is 1-9. In some embodiments, c is 1-8. In some embodiments, c is 1-7. In some embodiments, c is 1-6. In some embodiments, c is 1-5. In some embodiments, c is 1-4. In some embodiments, c is 1-3. In some embodiments, c is 1-2. In some embodiments, c is 1. In some embodiments, c is 2. In some embodiments, c is 3. In some embodiments, c is 4. In some embodiments, c is 5. In some embodiments, c is 6. In some embodiments, c is 7. In some embodiments, c is 8. In some embodiments, c is 9.

In some embodiments, each target binding moiety in an agent is the same. In some embodiments, each linker moiety connecting a target binding moiety to an antibody moiety is the same. In some embodiments, TBT in an agent are the same. In some embodiments, -L-(TBT)_{b} are the same.

In some embodiments, b is 1. In some embodiments, c is 1. In some embodiments, c is two or more. In some embodiments, c is 2. Those skilled in the art appreciate that various technologies can be utilized to conjugate antibody moieties with target binding moieties (e.g., certain technologies utilized for preparing antibody-drug conjugates) in accordance with the present disclosure. In some embodiments, target binding moieties are connected to antibody moieties through certain types of groups and/or amino acid residues. For example, in some embodiments, target binding moieties are connected to lysine residues optionally through linker moieties. In some embodiments, target binding moieties are connected to cysteine residues optionally through linker moieties. In some embodiments, target binding moieties are connected to unnatural amino acid residues optionally through linker moieties. In some embodiments, the present disclosure provides technologies for selectively linking target binding moieties to certain particular amino acid residues optionally through linker moieties. In some embodiments, provided technologies selectively connect target binding moieties to certain types of amino acid residues, e.g., lysine residues, optionally through linker moieties. In some embodiments, provided technologies selectively connect target binding moieties to particular sites of antibody moieties optionally through linker moieties. In some embodiments, provided technologies selectively connect target binding moieties to certain types of amino acid residues at particular sites optionally through linker moieties. For example, in some embodiments, target binding moieties are connected to K246 and K248 of an IgG1 heavy chain and amino acid residues corresponding thereto optionally through linker moieties. For example, in some embodiments, target binding moieties are connected to K251 and K253 of an IgG2 heavy chain and amino acid residues corresponding thereto optionally through linker moieties. For example, in some embodiments, target binding moieties are connected to K239 and K241 of an IgG4 heavy chain and amino acid residues corresponding thereto optionally through linker moieties. In some embodiments, a target binding moiety is connected to a particular amino acid residue or site optionally through a linker. In some embodiments, each target binding moiety is independently connected to a particular amino acid residue or site optionally through a linker. As appreciated by those skilled in the art, an antibody agent may comprise more than one particular sites (e.g., one on each of the more than one chain (e.g., one or each heavy chain)). In some embodiments, an antibody moiety comprise two heavy chains and one or both of the amino acid residues or amino acid residues corresponding thereto are each independently connected to a target binding moiety optionally through a linker. In some embodiments, one and no more than one is connected. In some embodiments, c is 1. In some embodiments, both are connected. In some embodiments, c is 2. In some embodiments, both target binding moieties and/or both linker moieties (if any) are the same.

In some embodiments, an agent is or comprises a Fc region. In some embodiments, an agent interacts hFcγRIIIA. In some embodiments, an agent interacts hFcγRIIIA on macrophages. In some embodiments, an agent recruit macrophages. In some embodiments, an agent interacts hFcγRIIA. In some embodiments, an agent interacts hFcγRIIA on dendritic cells. In some embodiments, an agent recruit dendritic cells. In some embodiments, an agent recruit NK cells.

Various antibody moieties, target binding moieties and linker moieties may be utilized in accordance with the present disclosure. Certain such moieties are described herein.

### Antibody Moieties

Various antibody moieties may be utilized in accordance with the present disclosure. In some embodiments, an antibody moiety is of an IgG antibody or a fragment thereof. In some embodiments, an antibody moiety is of an antibody in IVIG or a fragment thereof. In some embodiments, an antibody moiety is of a monoclonal antibody. In some embodiments, an antibody moiety is of an antibody in a polyclonal antibody composition or a fragment thereof. In some embodiments, an antibody moiety is of IgG or a fragment thereof. In some embodiments, an antibody moiety is of IgG1 or a fragment thereof. In some embodiments, an antibody moiety is of IgG2 or a fragment thereof. In some embodiments, an antibody moiety is of IgG3 or a fragment thereof. In some embodiments, an antibody moiety is of IgG4 or a fragment thereof. In some embodiments, an antibody moiety is of IgM or a fragment thereof.

In some embodiments, an antibody moiety is of a fragment of an antibody, e.g., of an IgG antibody, an IgM antibody, etc. In some embodiments, a fragment performs one or more functions of a full antibody. In some embodiments, a fragment recruits one or more or all immune activities typically recruited by a full antibody. In some embodiments, an antibody moiety is or comprises a Fc region. In some embodiments, an antibody moiety interacts hFcγRIIIA. In some embodiments, an antibody moiety interacts hFcγRIIIA on macrophages. In some embodiments, an antibody moiety recruit macrophages. In some embodiments, an antibody moiety interacts hFcγRIIA. In some embodiments, an antibody moiety interacts hFcγRIIA on dendritic cells. In some embodiments, an antibody moiety recruit dendritic cells. In some embodiments, an antibody moiety recruit NK cells.

### Arms

In some embodiments, the present disclosure provide an agent comprising:
an antibody binding moiety,
a target binding moiety, and
optionally a linker moiety.

In some embodiments, an agent comprising an antibody binding moiety, a target binding moiety and optionally a linker moiety which links an antibody binding moiety and a target binding moiety is referred to as an ARM agent or ARM.

In some embodiments, an agent contains one and only one antibody binding moiety.

In some embodiments, an antibody binding moiety is a uABT (universal antibody binding moiety) which can bind to antibodies against different antigens as described herein. In some embodiments, an antibody binding moiety can bind to two or more antibodies which have different Fab regions. In some embodiments, an antibody binding moiety can bind to two or more antibodies which have different antigens. In some embodiments, an antibody binding moiety can bind to Fc regions.

In some embodiments, the present disclosure provides agents that can bind to a SARS-CoV-2 spike protein or a fragment thereof. In some embodiments, an agent is a compound of formula M-I, M-II, I, I-a, I-b, II, or III, or a salt thereof. In some embodiments, the present disclosure provides compounds of formula M-I, M-II, I, I-a, I-b, II, or III, or pharmaceutically acceptable salts thereof. Various embodiments of provided technologies are described herein as examples.

In some embodiments, linker moieties of ARM agents comprise reactive groups, and are useful for preparing MATEs, e.g., through reactions with antibody agents. In some embodiments, the present disclosure provides technologies that can selectively conjugate antibody moieties, independently and optionally through linker moieties, to particular amino acid residues and/or sites of antibody agents. In some embodiments, an ARM agent is an agent having the structure of formula R-I or a salt thereof described herein.

Various antibody moieties, target binding moieties and linker moieties may be utilized in accordance with the present disclosure. Certain such moieties are described herein.

### Antibody Binding Moieties

In some embodiments, targets are antibody agents. In some embodiments, antibody binding moieties are antibody binding moieties. In some embodiments, provided compounds and/or agents comprise antibody binding moieties. Various antibody binding moieties can be utilized in accordance with the present disclosure. In some embodiments, antibody binding moieties are universal antibody binding moieties which can bind to antibodies having different Fab regions and different specificity. Among other things, agents/compounds comprising such antibody binding moieties may be utilized for conjugation with antibodies having different specificity. In some embodiments, antibody binding moieties of the present disclosure, e.g., universal antibody binding moieties, bind to Fc regions. In some embodiments, binding of antibody binding moieties to Fc regions can happen at the same time as binding of Fc receptors, e.g., CD16a, to the same Fc regions (e.g., may at different locations/amino acid residues of the same Fc regions). In some embodiments, upon binding of antibody binding moieties, e.g., those in provided agents, compounds, methods, etc., an Fc region can still interact with Fc receptors and perform one or more or all of its immune activities, including recruitment of immune cells (e.g., effector cells such as NK cells), and/or triggering, generating, encouraging, and/or enhancing immune system activities toward target cells, tissues, objects and/or entities, for example, antibody-dependent cell-mediated cytotoxicity (ADCC) and/or antibody-dependent cellular phagocytosis (ADCP).

Various antibody binding moieties including universal antibody binding moieties can be utilized in accordance with the present disclosure. Certain antibody binding moieties and technologies for identifying and/or assessing antibody binding moieties are described in WO2019/023501 and WO2019/136442, and are incorporated herein by reference. Those skilled in the art appreciates that additional technologies in the art may be suitable for identifying and/or assessing antibody binding moieties in accordance with the present disclosure. In some embodiments, an antibody binding moiety comprises one or more amino acid residues, each independently natural or unnatural.

In some embodiments, an antibody binding moiety, e.g., a protein binding moiety (e.g., an antibody binding moiety (e.g., a universal antibody binding moiety)), has the structure of

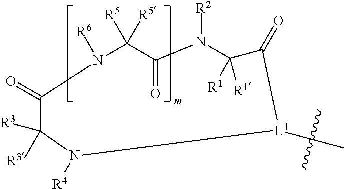

or a salt form thereof, wherein:
each of R¹, R³ and R⁵ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or:
   R¹ and R^{1′} are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur;
   R³ and R^{3′} are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur;
   an R⁵ group and the R^{5′} group attached to the same carbon atom are optionally taken together with their intervening carbon atom to form a 3-8 membered optionally substituted saturated or partially unsaturated spirocyclic carbocyclic ring or a 3-8 membered saturated or partially unsaturated spirocyclic heterocyclic ring having 1-2 heteroatoms independently selected from nitrogen, oxygen, or sulfur; or
   two R⁵ groups are optionally taken together with their intervening atoms to form a C₁₋₁₀ optionally substituted bivalent straight or branched saturated or unsaturated hydrocarbon chain wherein 1-3 methylene units of the chain are independently and optionally replaced with —S—, —SS—, —N(R)—, —O—, —C(O)—, —OC(O)—, —C(O)O—, —C(O)N(R)—, —N(R)C(O)—, —S(O)—, —S(O)₂—, or -Cy¹-, wherein each -Cy¹- is independently a 5-6 membered heteroarylenyl with 1-4 heteroatoms independently selected from nitrogen, oxygen or sulfur;
each of R^{1′}, R^{3′} and R^{5′} is independently hydrogen or optionally substituted C₁₋₃ aliphatic;
each of R², R⁴ and R⁶ is independently hydrogen, or optionally substituted C₁₋₄ aliphatic, or:
   R² and R¹ are optionally taken together with their intervening atoms to form a 4-8 membered, optionally substituted saturated or partially unsaturated monocyclic heterocyclic ring having 1-2 heteroatoms independently selected from nitrogen, oxygen, or sulfur;
   R⁴ and R³ are optionally taken together with their intervening atoms to form a 4-8 membered optionally substituted saturated or partially unsaturated monocyclic heterocyclic ring having 1-2 heteroatoms independently selected from nitrogen, oxygen, or sulfur; or
   an R⁶ group and its adjacent R⁵ group are optionally taken together with their intervening atoms to form a 4-8 membered optionally substituted saturated or partially unsaturated monocyclic heterocyclic ring having 1-2 heteroatoms independently selected from nitrogen, oxygen, or sulfur;
L¹ is a trivalent linker moiety; and
each of m and n is independently 1, 2, 3, 4, 5, 6, 7, 8, 9, 10, 11, 12, 13, 14, 15, 16, 17, 18, 19, or 20.

In some embodiments, L¹ is an optionally substituted trivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments L¹ is —(CH₂CH₂O)₂₋₄— or —(CH₂CH₂O)₂—.

In some embodiments, an antibody binding moiety, e.g. a protein binding moiety (e.g., an antibody binding moiety (e.g., a universal antibody binding moiety)), has the structure of

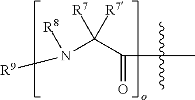

or a salt form thereof, wherein:
each of R⁷ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or:
   an R⁷ group and the R^{7′} group attached to the same carbon atom are optionally taken together with their intervening carbon atom to form a 3-8 membered optionally substituted saturated or partially unsaturated spirocyclic carbocyclic ring or a 3-8 membered optionally substituted saturated or partially unsaturated spirocyclic heterocyclic ring having 1-2 heteroatoms independently selected from nitrogen, oxygen, or sulfur;
each of R^{7′} is independently hydrogen or optionally substituted C₁₋₃ aliphatic;
each of R⁸ is independently hydrogen, or optionally substituted C₁₋₄ aliphatic, or:
   an R⁸ group and its adjacent R⁷ group are optionally taken together with their intervening atoms to form a 4-8 membered optionally substituted saturated or partially unsaturated monocyclic heterocyclic ring having 1-2 heteroatoms independently selected from nitrogen, oxygen, or sulfur; and
R⁹ is hydrogen, optionally substituted C₁₋₃ aliphatic, or —C(O)—.

In some embodiments, an antibody binding moiety, e.g., a universal antibody binding moiety is or comprises a peptide moiety, e.g., a moiety having the structure of R^{c}-(Xaa)z- or a salt form thereof, wherein each of R, z and Xaa is independently as described herein. In some embodiments, one or more Xaa are independently an unnatural amino acid residue. In some embodiments, side chains of two or more amino acid residues may be linked together to form bridges. For example, in some embodiments, side chains of two cysteine residues may form a disulfide bridge comprising —S—S— (which, as in many proteins, can be formed by two —SH groups).

In some embodiments, an antibody binding moiety, e.g. a protein binding moiety (e.g., an antibody binding moiety (e.g., a universal antibody binding moiety)), is or comprises a cyclic peptide moiety, e.g., a moiety having the structure of

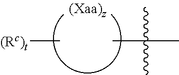

or a salt form thereof, wherein:
each Xaa is independently a residue of an amino acid or an amino acid analog;
t is 0-50;
z is 1-50;
each R^{c} is independently -L^{a}-R′;
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, a heteroatom is independently selected from oxygen, nitrogen, sulfur, phosphorus and silicon.

In some embodiments, an antibody binding moiety is or comprises R^{c}-(Xaa)z- or a salt form thereof, wherein each variable is as described herein. In some embodiments, a protein binding moiety is or comprises R^{c}-(Xaa)z- or a salt form thereof, wherein each variable is as described herein. In some embodiments, an antibody binding moiety, e.g., a universal antibody binding moiety, is or comprises R^{c}-(Xaa)z- or a salt form thereof, wherein each variable is as described herein. In some embodiments, an antibody binding moiety is or comprises

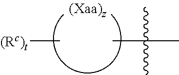

or a salt form thereof, wherein each variable is as described herein. In some embodiments, a protein binding moiety is or comprises

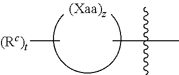

or a salt form thereof, wherein each variable is as described herein. In some embodiments, an antibody binding moiety, e.g., a universal antibody binding moiety, is or comprises

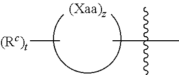

or a salt form thereof, wherein each variable is as described herein. In some embodiments, an antibody binding moiety, e.g., a universal antibody binding moiety is R^{c}-(Xaa)z- or

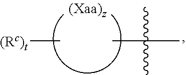

or a salt form thereof, and is or comprises a peptide unit. In some embodiments, -(Xaa)z- is or comprises a peptide unit. In some embodiments, amino acid residues may form bridges, e.g., connections formed by side chains optionally through linker moieties (e.g., L); for example, as in many polypeptides, cysteine residues may form disulfide bridges. In some embodiments, a peptide unit comprises an amino acid residue (e.g., at physiological pH about 7.4, “positively charged amino acid residue”, Xaa^{P}), e.g., a residue of an amino acid of formula A-I that has a positively charged side chain. In some embodiments, a peptide unit comprises R. In some embodiments, at least one Xaa is R. In some embodiments, a peptide unit is or comprises APAR (SEQ ID NO: 12). In some embodiments, a peptide unit is or comprises RAPA (SEQ ID NO: 13). In some embodiments, a peptide unit comprises an amino acid residue, e.g., a residue of an amino acid of formula A-I, that has a side chain comprising an aromatic group (“aromatic amino acid residue”, Xaa^{A}). In some embodiments, a peptide unit comprises a positively charged amino acid residue and an aromatic amino acid residue. In some embodiments, a peptide unit comprises W. In some embodiments, a peptide unit comprises a positively charged amino acid residue and an aromatic amino acid residue. In some embodiments, a peptide unit is or comprises Xaa^{A}XaaXaa^{P}Xaa^{p} (SEQ ID NO: 14). In some embodiments, a peptide unit is or comprises Xaa^{P}Xaa^{P}XaaXaa^{A} (SEQ ID NO: 15). In some embodiments, a peptide unit is or comprises Xaa^{P}Xaa^{A}Xaa^{P}. In some embodiments, a peptide unit is or comprises two or more Xaa^{P}Xaa^{A}Xaa^{P}. In some embodiments, a peptide unit is or comprises Xaa^{P}Xaa^{A}Xaa^{P}XaaXaa^{P}Xaa^{A}Xaa^{P} (SEQ ID NO: 16). In some embodiments, a peptide unit is or comprises Xaa^{P}Xaa^{P}Xaa^{A}Xaa^{A}Xaa^{p} (SEQ ID NO:17). In some embodiments, a peptide unit is or comprises Xaa^{P}Xaa^{P}Xaa^{P}Xaa^{A} (SEQ ID NO: 18). In some embodiments, a peptide unit is or comprises two or more Xaa^{A}Xaa^{A}Xaa^{P}. In some embodiments, a peptide residue comprises one or more proline residues. In some embodiments, a peptide unit is or comprises HWRGWA (SEQ ID NO: 19). In some embodiments, a peptide unit is or comprises WGRR (SEQ ID NO:20). In some embodiments, a peptide unit is or comprises RRGW (SEQ ID NO:21). In some embodiments, a peptide unit is or comprises NKFRGKYK (SEQ ID NO:22). In some embodiments, a peptide unit is or comprises NRFRGKYK (SEQ ID NO:23). In some embodiments, a peptide unit is or comprises NARKFYK (SEQ ID NO:24). In some embodiments, a peptide unit is or comprises NARKFYKG (SEQ ID NO:25). In some embodiments, a peptide unit is or comprises HWRGWV (SEQ ID NO:26). In some embodiments, a peptide unit is or comprises KHFRNKD (SEQ ID NO:27). In some embodiments, a peptide unit comprises a positively charged amino acid residue, an aromatic amino acid residue, and an amino acid residue, e.g., a residue of an amino acid of formula A-I, that has a negatively charged side chain (e.g., at physiological pH about 7.4, “negatively charged amino acid residue”, Xaa^{N}). In some embodiments, a peptide unit comprises RHRFNKD (SEQ ID NO:28). In some embodiments, a peptide unit is RHRFNKD (SEQ ID NO:28). In some embodiments, a peptide unit comprises TY. In some embodiments, a peptide unit is TY. In some embodiments, a peptide unit comprises TYK. In some embodiments, a peptide unit is TYK. In some embodiments, a peptide unit comprises RTY. In some embodiments, a peptide unit is RTY. In some embodiments, a peptide unit comprises RTYK (SEQ ID NO:29). In some embodiments, a peptide unit is RTYK (SEQ ID NO:29). In some embodiments, a peptide unit is or comprises a sequence selected from PAM. In some embodiments, a peptide unit comprises WHL. In some embodiments, a peptide unit is WHL. In some embodiments, a peptide unit is or comprises WXL, wherein X is an amino acid residue as described herein, e.g., one suitable for connection with another moiety (e.g., an amino acid residue comprising —COOH or a salt or activated form thereof such as D, E, etc.). In some embodiments, a peptide unit comprises WDL. In some embodiments, a peptide unit is WDL. In some embodiments, a peptide unit comprises ELVW (SEQ ID NO:30). In some embodiments, a peptide unit is ELVW (SEQ ID NO:30). In some embodiments, a peptide unit comprises GELVW (SEQ ID NO:31). In some embodiments, a peptide unit is GELVW (SEQ ID NO:31). In some embodiments, a peptide unit is or comprises a sequence selected from AWHLGELVW (SEQ ID NO:32). In some embodiments, a peptide unit is or comprises AWHLGELVW (SEQ ID NO:32). In some embodiments, a peptide unit is or comprises a sequence selected from AWDLGELVW (SEQ ID NO:33). In some embodiments, a peptide unit is or comprises AWDLGELVW (SEQ ID NO:33). In some embodiments, a peptide unit is or comprises AWXLGELVW (SEQ ID NO:34), wherein X is an amino acid residue as described herein, e.g., one suitable for connection with another moiety (e.g., an amino acid residue comprising —COOH or a salt or activated form thereof such as D, E, etc.). In some embodiments, a peptide unit is or comprises a sequence selected from DCAWHLGELVWCT (SEQ ID NO:35), wherein the two cysteine residues can form a disulfide bond as found in natural proteins. In some embodiments, a peptide unit is or comprises DCAWHLGELVWCT (SEQ ID NO:35), wherein the two cysteine residues can form a disulfide bond as found in natural proteins. In some embodiments, a peptide unit is or comprises a sequence selected from DCAWXLGELVWCT (SEQ ID NO:36), wherein the two cysteine residues can form a disulfide bond as found in natural proteins, and X is an amino acid residue as described herein, e.g., one suitable for connection with another moiety (e.g., an amino acid residue comprising —COOH or a salt or activated form thereof such as D, E, etc.). In some embodiments, a peptide unit is or comprises DCAWXLGELVWCT (SEQ ID NO:36), wherein the two cysteine residues can form a disulfide bond as found in natural proteins, and X is an amino acid residue as described herein, e.g., one suitable for connection with another moiety (e.g., an amino acid residue comprising —COOH or a salt or activated form thereof such as D, E, etc.). In some embodiments, X comprises —COOH or a salt or activated form thereof in its side chain. In some embodiments, a peptide unit is or comprises a sequence selected from DCAWDLGELVWCT (SEQ ID NO:37), wherein the two cysteine residues can form a disulfide bond as found in natural proteins. In some embodiments, a peptide unit is or comprises DCAWDLGELVWCT (SEQ ID NO:37), wherein the two cysteine residues can form a disulfide bond as found in natural proteins. In some embodiments, a peptide unit is or comprises a sequence selected from Fc-III. In some embodiments, a peptide unit is or comprises Fc-III. In some embodiments, a peptide unit is or comprises a sequence selected from ^{D}P^{L}PAWXLGELVW (SEQ ID NO:38), wherein X is an amino acid residue as described herein, e.g., one suitable for connection with another moiety (e.g., an amino acid residue comprising —COOH or a salt or activated form thereof such as D, E, etc.). In some embodiments, a peptide unit is or comprises ^{D}P^{L}PAWXLGELVW (SEQ ID NO:38), wherein X is an amino acid residue as described herein, e.g., one suitable for connection with another moiety (e.g., an amino acid residue comprising —COOH or a salt or activated form thereof such as D, E, etc.). In some embodiments, a peptide unit is or comprises a sequence selected from ^{D}P^{L}PAWDLGELVW (SEQ ID NO:39). In some embodiments, a peptide unit is or comprises ^{D}P^{L}PAWDLGELVW (SEQ ID NO:39). In some embodiments, a peptide unit is or comprises a sequence selected from ^{D}P^{L}PAWHLGELVW (SEQ ID NO:40), wherein the two cysteine residues can form a disulfide bond as found in natural proteins. In some embodiments, a peptide unit is or comprises ^{D}P^{L}PAWHLGELVW (SEQ ID NO:40) (e.g., FcBP-1), wherein the two cysteine residues can form a disulfide bond as found in natural proteins. In some embodiments, a peptide unit is or comprises a sequence selected from FcBP-1. In some embodiments, a peptide unit is or comprises a sequence selected from ^{D}P^{L}PDCAWXLGELVWCT (SEQ ID NO:41), wherein the two cysteine residues can form a disulfide bond as found in natural proteins, and X is an amino acid residue as described herein, e.g., one suitable for connection with another moiety (e.g., an amino acid residue comprising —COOH or a salt or activated form thereof such as D, E, etc.). In some embodiments, a peptide unit is or comprises ^{D}P^{L}PDCAWXLGELVWCT (SEQ ID NO:41), wherein the two cysteine residues can form a disulfide bond as found in natural proteins, and X is an amino acid residue as described herein, e.g., one suitable for connection with another moiety (e.g., an amino acid residue comprising —COOH or a salt or activated form thereof such as D, E, etc.). In some embodiments, a peptide unit is or comprises a sequence selected from ^{D}P^{L}PDCAWHLGELVWCT (SEQ ID NO:42), wherein the two cysteine residues can form a disulfide bond as found in natural proteins. In some embodiments, a peptide unit is or comprises ^{D}P^{L}PDCAWHLGELVWCT (SEQ ID NO:42) (e.g., FcBP-2), wherein the two cysteine residues can form a disulfide bond as found in natural proteins. In some embodiments, a peptide unit is or comprises a sequence selected from ^{D}P^{L}PDCAWDLGELVWCT (SEQ ID NO:43), wherein the two cysteine residues can form a disulfide bond as found in natural proteins. In some embodiments, a peptide unit is or comprises ^{D}P^{L}PDCAWDLGELVWCT (SEQ ID NO:43), wherein the two cysteine residues can form a disulfide bond as found in natural proteins. In some embodiments, a peptide unit is or comprises a sequence selected from FcBP-2. In some embodiments, a peptide unit is or comprises a sequence selected from CDCAWXLGELVWCTC (SEQ ID NO:44), wherein the first and the last cysteines, and the two cysteines in the middle of the sequence, can each independently form a disulfide bond as in natural proteins, and X is an amino acid residue as described herein, e.g., one suitable for connection with another moiety (e.g., an amino acid residue comprising —COOH or a salt or activated form thereof such as D, E, etc.). In some embodiments, a peptide unit is or comprises CDCAWXLGELVWCTC (SEQ ID NO:44), wherein the first and the last cysteines, and the two cysteines in the middle of the sequence, can each independently form a disulfide bond as in natural proteins, and X is an amino acid residue as described herein, e.g., one suitable for connection with another moiety (e.g., an amino acid residue comprising —COOH or a salt or activated form thereof such as D, E, etc.). In some embodiments, a peptide unit is or comprises a sequence selected from CDCAWHLGELVWCTC (SEQ ID NO:45), wherein the first and the last cysteines, and the two cysteines in the middle of the sequence, can each independently form a disulfide bond as in natural proteins. In some embodiments, a peptide unit is or comprises CDCAWHLGELVWCTC (SEQ ID NO:45), wherein the first and the last cysteines, and the two cysteines in the middle of the sequence, can each independently form a disulfide bond as in natural proteins. In some embodiments, a peptide unit is or comprises a sequence selected from CDCAWDLGELVWCTC (SEQ ID NO:46), wherein the first and the last cysteines, and the two cysteines in the middle of the sequence, can each independently form a disulfide bond as in natural proteins. In some embodiments, a peptide unit is or comprises CDCAWDLGELVWCTC (SEQ ID NO:46), wherein the first and the last cysteines, and the two cysteines in the middle of the sequence, can each independently form a disulfide bond as in natural proteins. In some embodiments, a peptide unit is or comprises a sequence selected from Fc-III-4c. In some embodiments, a peptide unit is or comprises a sequence selected from FcRM. In some embodiments, a peptide unit is or comprises a cyclic peptide unit. In some embodiments, a cyclic peptide unit comprises amide group formed by an amino group of a side chain and the C-terminus —COOH. It is appreciated by those skilled in the art that in various embodiments, when a peptide unit is connected to another moiety, an amino acid residue of a peptide unit may be connected through various positions, e.g., its backbone, its side chain, etc. In some embodiments, an amino acid residue is modified for connection. In some embodiments, an amino acid residue is replaced with another suitable residue for connection while maintaining one or more properties and/or activities a peptide unit (e.g., binding to an antibody as described herein). For example, in some embodiments, an amino acid residue is replaced with an amino acid residue with a side chain comprising —COOH or a salt or activated form thereof (e.g., side chain being —CH₂—COOH or a salt or activated form thereof). As exemplified herein, in various sequences H may be replaced with D (e.g., in various peptide units comprising WHL). In some embodiments, a peptide unit is connected to another moiety through —COOH or a salt or activated form thereof, e.g., through formation of e.g., —CON(R′)—. In some embodiments, R′ is —H. In some embodiments, —COOH is in a side chain of an amino acid residue. In some embodiments, in a sequence described herein (e.g., DCAWHLGELVWCT, SEQ ID NO:35), 1-5 (e.g., 1, 2, 3, 4, or 5) amino acid residues may be independently and optionally replaced with another amino acid residue, 1-5 (e.g., 1, 2, 3, 4, or 5) amino acid residues may be independently and optionally deleted, and/or 1-5 (e.g., 1, 2, 3, 4, or 5) amino acid residues may be independently and optionally inserted. In some embodiments, a peptide moiety is connected to the rest of a molecule through its N-terminus. In some embodiments, it is connected to the rest of a molecule through its C-terminus. In some embodiments, it is connected to the rest of a molecule through a side chain of an amino acid residue (e.g., various X residues as described in the present disclosure). In some embodiments, two cysteine residues may independently and optionally form a disulfide bond. In some embodiments, the total number of replacements, deletions and insertions is no more than 10 (e.g., 0, or no more than 1, 2, 3, 4, 5, 6, 7, 8, 9, or 10). In some embodiments, the total number is 0. In some embodiments, the total number is no more than 1. In some embodiments, the total number is no more than 2. In some embodiments, the total number is no more than 3. In some embodiments, the total number is no more than 4. In some embodiments, the total number is no more than 5. In some embodiments, the total number is no more than 6. In some embodiments, the total number is no more than 7. In some embodiments, the total number is no more than 8. In some embodiments, the total number is no more than 9. In some embodiments, the total number is no more than 10. In some embodiments, there are no insertions. In some embodiments, there are no deletions.

In some embodiments, an antibody binding moiety comprises or has the structure of DCAWHLGELVWCT (SEQ ID NO:35) or a salt form thereof, wherein the two C residues are linked by a —S—S—. In some embodiments, an antibody binding moiety comprises or has the structure of DCAWHLGELVWCT (SEQ ID NO:35) or a salt form thereof, wherein the N-terminus is capped with R—C(O)—. In some embodiments, wherein R is methyl. In some embodiments, an antibody binding moiety is connected to the rest of a molecule through its C-terminus.

In some embodiments, -(Xaa)z- is or comprises [X¹]ₚ₁[X²]ₚ₂—X³X⁴X⁵X⁶X⁷X⁸X⁹X¹⁰X¹¹X¹²—[X¹³]ₚ₁₃—[X¹⁴]ₚ₁₄[X¹⁵]ₚ₁₅[X¹⁶]ₚ₁₆, wherein each of X¹, X², X³, X⁴, X⁵, X⁶, X⁷, X⁸, X⁹, X¹⁰, X¹¹, X¹², and X¹³ is independently an amino acid residue, e.g., of an amino acid of formula A-I, and each of p1, p2, p13, p14, p15 and p16 is independently 0, 1, 2, 3, 4, 5, 6, 7, 8, 9, or 10. In some embodiments, each of X¹, X², X³, X⁴, X⁵, X⁶, X⁷, X⁸, X⁹, X¹⁰, X¹¹, X¹², and X¹³ is independently an amino acid residue of an amino acid of formula A-I. In some embodiments, each of X¹, X², X³, X⁴, X⁵, X⁶, X⁷, X⁸, X⁹, X¹⁰, X¹¹, X¹², and X¹³ is independently a natural amino acid residue. In some embodiments, one or more of X¹, X², X³, X⁴, X⁵, X⁶, X⁷, X⁸, X⁹, X¹⁰, X¹¹, X¹², and X¹³ are independently an unnatural amino acid residue as described in the present disclosure.

In some embodiments, a peptide unit comprises a functional group in an amino acid residue that can react with a functional group of another amino acid residue. In some embodiments, a peptide unit comprises an amino acid residue with a side chain which comprises a functional group that can react with another functional group of the side chain of another amino acid residue to form a linkage (e.g., see moieties described in Table A-1, Table 1, etc.). In some embodiments, one functional group of one amino acid residue is connected to a functional group of another amino acid residue to form a linkage (or bridge). Linkages are bonded to backbone atoms of peptide units and comprise no backbone atoms. In some embodiments, a peptide unit comprises a linkage formed by two side chains of non-neighboring amino acid residues. In some embodiments, a linkage is bonded to two backbone atoms of two non-neighboring amino acid residues. In some embodiments, both backbone atoms bonded to a linkage are carbon atoms. In some embodiments, a linkage has the structure of L^{b}, wherein L^{b} is L^{a} as described in the present disclosure, wherein L^{a} is not a covalent bond. In some embodiments, L^{a} comprises -Cy-. In some embodiments, L^{a} comprises -Cy-, wherein -Cy- is optionally substituted heteroaryl. In some embodiments, -Cy- is

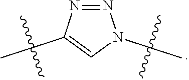 [SMILES: *c1cn(*)nn1]

In some embodiments, L^{a} is

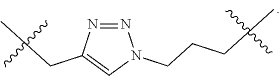 [SMILES: *CCCn1cc(C*)nn1]

In some embodiments, such an L^{a} can be formed by a —N₃ group of the side chain of one amino acid residue, and the -≡- of the side chain of another amino acid residue. In some embodiments, a linkage is formed through connection of two thiol groups, e.g., of two cysteine residues. In some embodiments, L^{a} comprises —S—S—. In some embodiments, L^{a} is —CH₂—S—S—CH₂—. In some embodiments, a linkage is formed through connection of an amino group (e.g., —NH₂ in the side chain of a lysine residue) and a carboxylic acid group (e.g., —COOH in the side chain of an aspartic acid or glutamic acid residue). In some embodiments, L^{a} comprises —C(O)—N(R′)—. In some embodiments, L^{a} comprise —C(O)—NH—. In some embodiments, L^{a} is —CH₂CONH—(CH₂)₃—. In some embodiments, L^{a} comprises —C(O)—N(R′)—, wherein R′ is R, and is taken together with an R group on the peptide backbone to form a ring (e.g., in A-34). In some embodiments, L^{a} is —(CH₂)₂—N(R′)—CO—(CH₂)₂—. In some embodiments, -Cy- is optionally substituted phenylene. In some embodiments, -Cy- is optionally substituted 1,2-phenylene. In some embodiments, L^{a} is

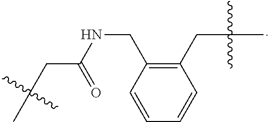 [SMILES: *CC(=O)NCc1ccccc1C*]

In some embodiments, L^{a} is

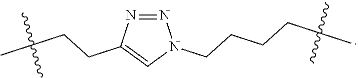 [SMILES: *CCCCn1cc(CC*)nn1]

In some embodiments, L^{a} is optionally substituted bivalent C₂₋₂₀ bivalent aliphatic. In some embodiments, L^{a} is optionally substituted —(CH₂)₉—CH═CH—(CH₂)₉—. In some embodiments, L^{a} is —(CH₂)₃—CH═CH—(CH₂)₃—.

In some embodiments, two amino acid residues bonded to a linkage are separated by 1, 2, 3, 4, 5, 6, 7, 8, 9, 10, 11, 12, 13, 14, 15, or more than 15 amino acid residues between them (excluding the two amino acid residues bonded to the linkage). In some embodiments, the number is 1. In some embodiments, the number is 2. In some embodiments, the number is 3. In some embodiments, the number is 4. In some embodiments, the number is 5. In some embodiments, the number is 6. In some embodiments, the number is 7. In some embodiments, the number is 8. In some embodiments, the number is 9. In some embodiments, the number is 10. In some embodiments, the number is 11. In some embodiments, the number is 12. In some embodiments, the number is 13. In some embodiments, the number is 14. In some embodiments, the number is 15.

In some embodiments, each of p1, p2, p13, p14, p15 and p16 is 0. In some embodiments, -(Xaa)z- is or comprises —X³X⁴X⁵X⁶X⁷X⁸X⁹X¹⁰X¹¹X¹²—, wherein:
each of X³, X⁴, X⁵, X⁶, X⁷, X⁸, X⁹, X¹⁰, X¹¹, and X¹² is independently an amino acid residue;
X⁶ is Xaa^{A} or Xaa^{P};
X⁹ is Xaa^{N}; and
X¹² is Xaa^{A} or Xaa^{P}.

In some embodiments, each of X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, X⁵ is Xaa^{A} or Xaa^{P}. In some embodiments, X⁵ is Xaa^{A}. In some embodiments, X⁵ is Xaa^{P}. In some embodiments, X⁵ is an amino acid residue whose side chain comprises an optionally substituted saturated, partially saturated or aromatic ring. In some embodiments, X⁵ is

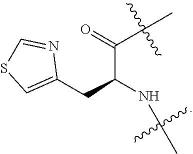 [SMILES: *N[C@@H](Cc1cscn1)C(*)=O]

In some embodiments, X⁵ is

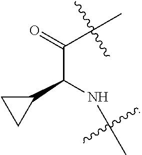 [SMILES: *N[C@H](C(*)=O)C1CC1]

In some embodiments, X⁶ is Xaa^{A}. In some embodiments, X⁶ is Xaa^{P}. In some embodiments, X⁶ is His. In some embodiments, X¹² is Xaa^{A}. In some embodiments, X¹² is Xaa^{P}. In some embodiments, X⁹ is Asp. In some embodiments, X⁹ is Glu. In some embodiments, X¹² is

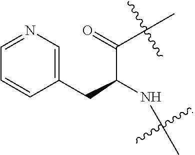 [SMILES: *N[C@@H](Cc1cccnc1)C(*)=O]

In some embodiments, X¹² is

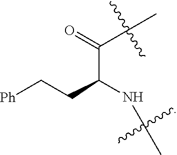 [SMILES: *N[C@@H](CCc1ccccc1)C(*)=O]

In some embodiments, each of X⁷, X¹⁰, and X¹¹ is independently an amino acid residue with a hydrophobic side chain (“hydrophobic amino acid residue”, Xaa^{H}). In some embodiments, X⁷ is Xaa^{H}. In some embodiments, X⁷ is

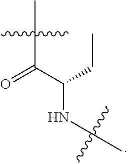 [SMILES: *N[C@@H](CC)C(*)=O]

In some embodiments, X⁷ is Val. In some embodiments, X¹⁰ is Xaa^{H}. In some embodiments, X¹⁰ is Met. In some embodiments, X¹⁰ is

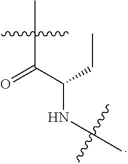 [SMILES: *N[C@@H](CC)C(*)=O]

In some embodiments, X¹¹ is Xaa^{H}. In some embodiments, X¹¹ is

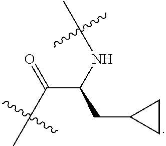 [SMILES: *N[C@@H](CC1CC1)C(*)=O]

In some embodiments, X⁸ is Gly. In some embodiments, X⁴ is Pro. In some embodiments, X³ is Lys. In some embodiments, the —COOH of X¹² forms an amide bond with the side chain amino group of Lys (X³), and the other amino group of the Lys (X³) is connected to a linker moiety and then an antibody binding moiety.

In some embodiments, -(Xaa)z- is or comprises —X³X⁴X⁵X⁶X⁷X⁸X⁹X¹⁰X¹¹X¹²—, wherein:
each of X³, X⁴, X⁵, X⁶, X⁷, X⁸, X⁹, X¹⁰, X¹¹, and X¹² is independently an amino acid residue;
at least two amino acid residues are connected through one or more linkages L^{b};
L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁶ is Xaa^{A} or Xaa^{P};
X⁹ is Xaa^{N}; and
X¹² is Xaa^{A} or Xaa^{P}.

In some embodiments, each of X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, X⁵ and X¹⁰ are connected by L^{b}. In some embodiments, there is one linkage L^{b}. In some embodiments, X⁶ is Xaa^{A}. In some embodiments, X⁶ is Xaa^{P}. In some embodiments, X⁶ is His. In some embodiments, X⁹ is Asp. In some embodiments, X⁹ is Glu. In some embodiments, X¹² is Xaa^{A}. In some embodiments, X¹² is

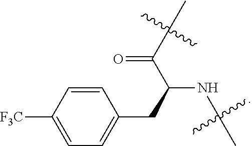 [SMILES: *N[C@@H](Cc1ccc(C(F)(F)F)cc1)C(*)=O]

In some embodiments, X¹² is

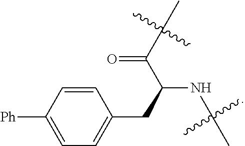 [SMILES: *N[C@@H](Cc1ccc(-c2ccccc2)cc1)C(*)=O]

In some embodiments, X¹² is

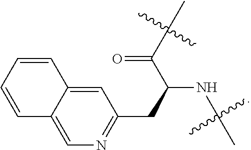 [SMILES: *N[C@@H](Cc1cc2ccccc2cn1)C(*)=O]

In some embodiments, each of X⁴, X⁷, and X¹¹ is independently Xaa^{H}. In some embodiments, X⁴ is Xaa^{H}. In some embodiments, X⁴ is Ala. In some embodiments, X⁷ is Xaa^{H}. In some embodiments, X⁷ is

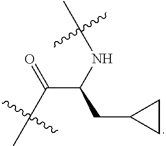 [SMILES: *N[C@@H](CC1CC1)C(*)=O]

In some embodiments, X¹¹ is Xaa^{H}. In some embodiments, X¹¹ is

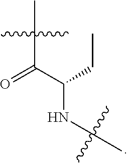 [SMILES: *N[C@@H](CC)C(*)=O]

In some embodiments, X⁸ is Gly. In some embodiments, X³ is Lys. In some embodiments, the —COOH of X¹² forms an amide bond with the side chain amino group of Lys (X³), and the other amino group of the Lys (X³) is connected to a linker moiety and then an antibody binding moiety. In some embodiments, L^{b} is

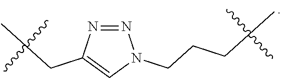 [SMILES: *CCCn1cc(C*)nn1]

In some embodiments, L^{b} is

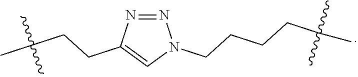 [SMILES: *CCCCn1cc(CC*)nn1]

In some embodiments, L^{b} connects two alpha-carbon atoms of two different amino acid residues. In some embodiments, both X⁵ and X¹⁰ are Cys, and the two —SH groups of their side chains form —S—S— (L^{b} is —CH₂—S—S—CH₂—).

In some embodiments, -(Xaa)z- is or comprises —X²X³X⁴X⁵X⁶X⁷X⁸X⁹X¹⁰X¹¹X¹²—, wherein:
each of X², X³, X⁴, X⁵, X⁶, X⁷, X⁸, X⁹, X¹⁰, X¹¹, and X¹² is independently an amino acid residue;
at least two amino acid residues are connected through one or more linkages L^{b};
L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁴ is Xaa^{A};
X⁵ is Xaa^{A} or Xaa^{P};
X⁸ is Xaa^{N}; and
X¹¹ is Xaa^{A}.

In some embodiments, each of X², X³, X⁴, X⁵, X⁶, X⁷, X¹, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, there is one linkage L^{b}. In some embodiments, X² and X¹² are connected by L^{b}. In some embodiments, L^{b} is —CH₂—S—S—CH₂—. In some embodiments, L^{b} is —CH₂—CH₂—S—CH₂—. In some embodiments, L is

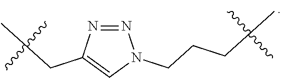 [SMILES: *CCCn1cc(C*)nn1]

In some embodiments, L^{b} is

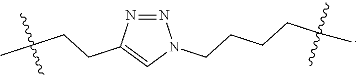 [SMILES: *CCCCn1cc(CC*)nn1]

In some embodiments, L^{b} is —CH₂CH₂CO—N(R′)—CH₂CH₂—. In some embodiments, R′ are taken together with an R group on the backbone atom that —N(R′)—CH₂CH₂— is bonded to form a ring, e.g., as in A-34. In some embodiments, a formed ring is 3-, 4-, 5-, 6-, 7- or 8-membered. In some embodiments, a formed ring is monocyclic. In some embodiments, a formed ring is saturated. In some embodiments, L^{b} is

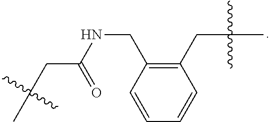 [SMILES: *CC(=O)NCc1ccccc1C*]

In some embodiments, L^{b} connects two alpha-carbon atoms of two different amino acid residues. In some embodiments, X⁴ is Xaa^{A}. In some embodiments, X⁴ is Tyr. In some embodiments, X⁵ is Xaa^{A}. In some embodiments, X⁵ is Xaa^{P}. In some embodiments, X⁵ is His. In some embodiments, X⁸ is Asp. In some embodiments, X⁸ is Glu. X¹¹ is Tyr. In some embodiments, both X² and X¹² are Cys, and the two —SH groups of their side chains form —S—S— (L^{b} is —CH₂—S—S—CH₂—). In some embodiments, each of X³, X⁶, X⁹, and X¹⁰ is independently Xaa^{H}. In some embodiments, X³ is Xaa^{H}. In some embodiments, X³ is Ala. In some embodiments, X⁶ is Xaa^{H}. In some embodiments, X⁶ is Leu. In some embodiments, X⁹ is Xaa^{H}. In some embodiments, X⁹ is Leu. In some embodiments, X⁹ is

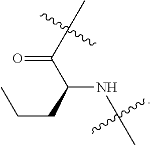 [SMILES: *N[C@@H](CCC)C(*)=O]

In some embodiments, X¹⁰ is Xaa^{H}. In some embodiments, X¹⁰ is Val. In some embodiments, X¹⁰ is

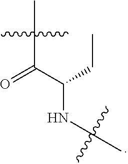 [SMILES: *N[C@@H](CC)C(*)=O]

In some embodiments, X⁷ is Gly. In some embodiments, p1 is 1. In some embodiments, X¹ is Asp. In some embodiments, p13 is 1. In some embodiments, p14, p15 and p16 are 0. In some embodiments, X³ is an amino acid residue comprising a polar uncharged side chain (e.g., at physiological pH, “polar uncharged amino acid residue”, Xaa^{L}). In some embodiments, X¹³ is Thr. In some embodiments, X¹³ is Val. In some embodiments, p13 is 0. In some embodiments, R^{c} is —NHCH₂CH(OH)CH₃. In some embodiments, R^{c} is (R)—NHCH₂CH(OH)CH₃. In some embodiments, R^{c} is (S)—NHCH₂CH(OH)CH₃.

In some embodiments, -(Xaa)z- is or comprises —X²X³X⁴X⁵X⁶X⁷X⁸X⁹X¹⁰, X¹¹X¹²—, wherein:
each of X², X³, X⁴, X⁵, X⁶, X⁷, X⁸, X⁹, X¹⁰, X¹¹, and X¹² is independently an amino acid residue;
at least two amino acid residues are connected through one or more linkages L^{b};
L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X⁵ is Xaa^{A} or Xaa^{P};
X⁸ is Xaa^{N}; and
X¹¹ is Xaa^{A}.

In some embodiments, each of X², X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, there is one linkage L^{b}. In some embodiments, there are two or more linkages L^{b}. In some embodiments, there are two linkages L^{b}. In some embodiments, X² and X¹² are connected by L^{b}. In some embodiments, X⁴ and X⁹ are connected by L^{b}. In some embodiments, X⁴ and X¹⁰ are connected by L^{b}. In some embodiments, L^{b} is —CH₂—S—S—CH₂—. In some embodiments, L^{b} is

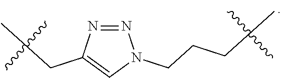 [SMILES: *CCCn1cc(C*)nn1]

In some embodiments, L^{b} is

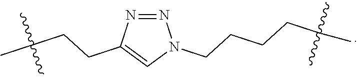 [SMILES: *CCCCn1cc(CC*)nn1]

In some embodiments, both X² and X¹² are Cys, and the two —SH groups of their side chains form —S—S— (L^{b} is —CH₂—S—S—CH₂—). In some embodiments, both X⁴ and X¹⁰ are Cys, and the two —SH groups of their side chains form —S—S— (L^{b} is —CH₂—S—S—CH₂—). In some embodiments, X⁴ and X⁹ are connected by L^{b}, wherein L^{b} is

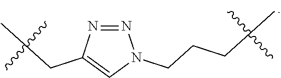 [SMILES: *CCCn1cc(C*)nn1]

In some embodiments, X⁴ and X⁹ are connected by L^{b}, wherein L^{b} is

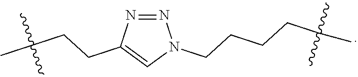 [SMILES: *CCCCn1cc(CC*)nn1]

In some embodiments, X⁵ is Xaa^{A}. In some embodiments, X⁵ is Xaa^{P}. In some embodiments, X⁵ is His. In some embodiments, X⁸ is Asp. In some embodiments, X⁸ is Glu. In some embodiments, X¹¹ is Tyr. In some embodiments, X¹¹ is

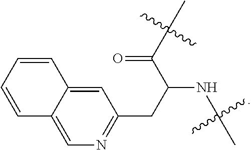 [SMILES: *NC(Cc1cc2ccccc2cn1)C(*)=O]

In some embodiments, X² and X¹² are connected by L^{b}, wherein L^{b} is —CH₂—S—CH₂CH₂—. In some embodiments, L^{b} connects two alpha-carbon atoms of two different amino acid residues. In some embodiments, each of X³, X⁶, and X⁹ is independently Xaa^{H}. In some embodiments, X³ is Xaa^{H}. In some embodiments, X³ is Ala. In some embodiments, X⁶ is Xaa^{H}. In some embodiments, X⁶ is Leu. In some embodiments, X⁶ is

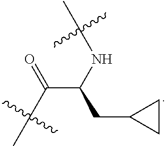 [SMILES: *N[C@@H](CC1CC1)C(*)=O]

In some embodiments, X⁹ is Xaa^{H}. In some embodiments, X⁹ is Leu. In some embodiments, X⁹ is

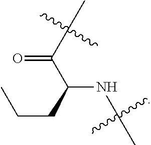 [SMILES: *N[C@@H](CCC)C(*)=O]

In some embodiments, X¹⁰ is Xaa^{H}. In some embodiments, X¹⁰ is Val. In some embodiments, X⁷ is Gly. In some embodiments, p1 is 1. In some embodiments, X is Xaa^{N} some embodiments, X¹ is Asp. In some embodiments, X¹ is Glu. In some embodiments, p13 is 1. In some embodiments, p14, p15 and p16 are 0. In some embodiments, X¹³ is Xaa^{L}. In some embodiments, X¹³ is Thr. In some embodiments, X¹³ is Val.

In some embodiments, -(Xaa)z- is or comprises —X²X³X⁴X⁵X⁶X⁷X⁸X⁹X¹⁰X¹¹X¹²X¹³X¹⁴X¹⁵X¹⁶—, wherein:
each of X², X³, X⁴, X⁵, X⁶, X⁷, X⁸, X⁹, X¹⁰, X¹¹, X¹², X¹³, X¹⁴, X¹⁵, and X¹⁶ is independently an amino acid residue;
at least two amino acid residues are connected through a linkage L^{b};
L^{b} is an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, wherein L^{b} is bonded to a backbone atom of one amino acid residue and a backbone atom of another amino acid residue, and comprises no backbone atoms;
X³ is Xaa^{N};
X⁶ is Xaa^{A};
X⁷ is Xaa^{A} or Xaa^{P};
X⁹ is Xaa^{N}; and
X¹³ is Xaa^{A}.

In some embodiments, each of X², X³, X⁴, X⁵, X⁶, X⁷, X⁸, X⁹, X¹⁰, X¹¹, and X¹² is independently an amino acid residue of an amino acid of formula A-I as described in the present disclosure. In some embodiments, two non-neighboring amino acid residues are connected by L^{b}. In some embodiments, there is one linkage L^{b}. In some embodiments, there are two or more linkages L. In some embodiments, there are two linkages L^{b}. In some embodiments, X² are connected to X¹⁶ by L^{b}. In some embodiments, X⁴ are connected to X¹⁴ by L^{b}. In some embodiments, both X² and X¹⁶ are Cys, and the two —SH groups of their side chains form —S—S-(L^{b} is —CH₂—S—S—CH₂—). In some embodiments, both X⁴ and X¹⁴ are Cys, and the two —SH groups of their side chains form —S—S— (L^{b} is —CH₂—S—S—CH₂—). In some embodiments, L^{b} connects two alpha-carbon atoms of two different amino acid residues. In some embodiments, X³ is Asp. In some embodiments, X³ is Glu. In some embodiments, X⁵ is Xaa^{H}. In some embodiments, X⁵ is Ala. In some embodiments, X⁶ is Xaa^{A}. In some embodiments, X⁶ is Tyr. In some embodiments, X⁷ is Xaa^{A}. In some embodiments, X⁷ is Xaa^{P}. In some embodiments, X⁷ is His. In some embodiments, X⁸ is Xaa^{H}. In some embodiments, X⁸ is Ala. In some embodiments, X⁹ is Gly. In some embodiments, X¹⁰ is Asp. In some embodiments, X¹⁰ is Glu. In some embodiments, X¹¹ is Xaa^{H}. In some embodiments, X¹¹ is Leu. In some embodiments, X¹² is Xaa^{H}. In some embodiments, X¹² is Val. In some embodiments, X¹³ is Xaa^{A}. In some embodiments, X¹³ is Tyr. In some embodiments, X¹⁵ is Xaa^{L}. In some embodiments, X¹⁵ is Thr. In some embodiments, X¹⁵ is Val. In some embodiments, p1 is 1. In some embodiments, In some embodiments, X¹ is Xaa^{N}. In some embodiments, X¹ is Asp. In some embodiments, X¹ is Glu.

As appreciated by those skilled in the art, an amino acid residue may be replaced by another amino acid residue having similar properties, e.g., one Xaa^{H} (e.g., Val, Leu, etc.) may be replaced with another Xaa^{H} (e.g., Leu, Ile, Ala, etc.), one Xaa^{A} may be replaced with another Xaa^{A}, one Xaa^{P} may be replaced with another Xaa^{p}, one Xaa^{N} may be replaced with another Xaa^{N}, one Xaa^{L} may be replaced with another Xaa^{L}, etc.

In some embodiments, an antibody binding moiety is or comprises optionally substituted moiety of Table A-1. In some embodiments, a protein binding moiety is or comprises optionally substituted moiety of Table A-1. In some embodiments, an antibody binding moiety, e.g., a universal antibody binding moiety, is or comprises optionally substituted moiety of Table A-1. In some embodiments, an antibody binding moiety is selected from able A-1. In some embodiments, a protein binding moiety is selected from able A-1. In some embodiments, an antibody binding moiety, e.g., a universal antibody binding moiety, is selected from able A-1. In some embodiments, C-terminus and/or N-terminus are optionally capped (e.g., for C-terminus, by converting —COOH into —C(O)N(R′)₂ like —C(O)NH₂; for N-terminus, by adding R′C(O)— like CH₃C(O)— to an amino group).

TABLE A-1
Exemplified antibody binding moieties.

| | |
|---|---|
| 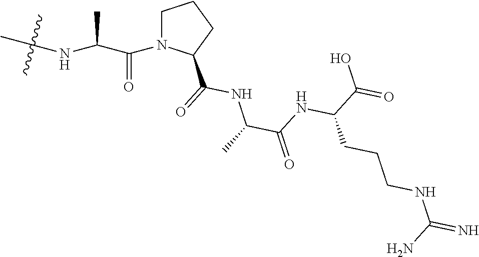 [SMILES: *N[C@@H](C)C(=O)N1CCC[C@H]1C(=O)N[C@@H](C)C(=O)N[C@@H](CCCNC(=N)N)C(=O)O] | A-1 |
| 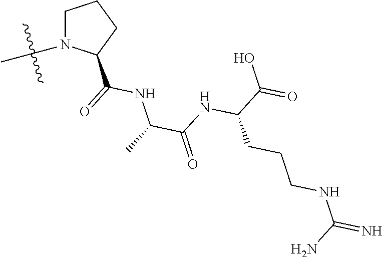 [SMILES: *N1CCC[C@H]1C(=O)N[C@@H](C)C(=O)N[C@@H](CCCNC(=N)N)C(=O)O] | A-2 |
| 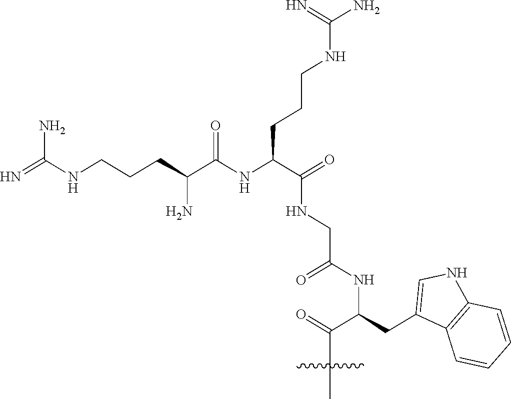 [SMILES: *C(=O)[C@H](Cc1c[nH]c2ccccc12)NC(=O)CNC(=O)[C@H](CCCNC(=N)N)NC(=O)[C@@H](N)CCCNC(=N)N] | A-3 |
| 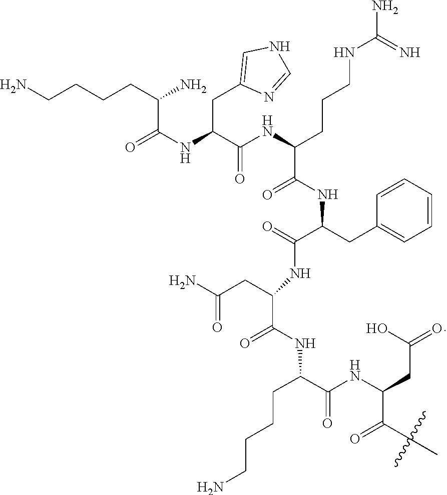 [SMILES: *C(=O)[C@H](CC(=O)O)NC(=O)[C@H](CCCCN)NC(=O)[C@H](CC(N)=O)NC(=O)[C@H](Cc1ccccc1)NC(=O)[C@H](CCCNC(=N)N)NC(=O)[C@H](Cc1c[nH]cn1)NC(=O)[C@@H](N)CCCCN] | A-4 |
| 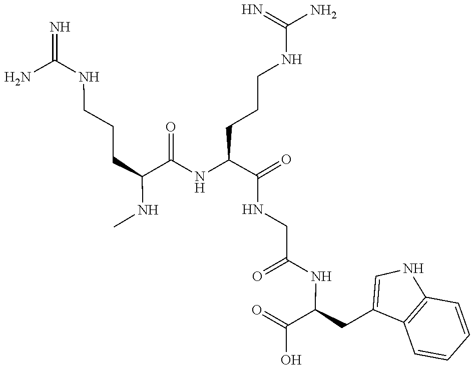 [SMILES: CN[C@@H](CCCNC(=N)N)C(=O)N[C@@H](CCCNC(=N)N)C(=O)NCC(=O)N[C@@H](Cc1c[nH]c2ccccc12)C(=O)O] | A-5 |
| 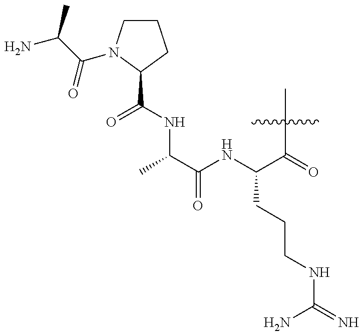 [SMILES: *C(=O)[C@H](CCCNC(=N)N)NC(=O)[C@H](C)NC(=O)[C@@H]1CCCN1C(=O)[C@H](C)N] | A-6 |
| 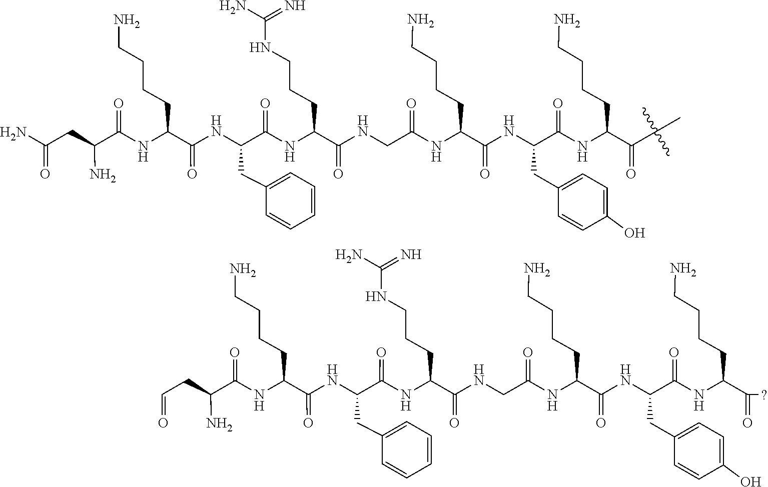 | A-7 |
| 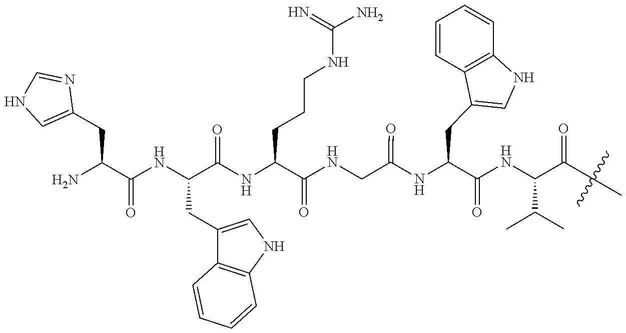 [SMILES: *C(=O)[C@@H](NC(=O)[C@H](Cc1c[nH]c2ccccc12)NC(=O)CNC(=O)[C@H](CCCNC(=N)N)NC(=O)[C@H](Cc1c[nH]c2ccccc12)NC(=O)[C@@H](N)Cc1c[nH]cn1)C(C)C] | A-8 |
| 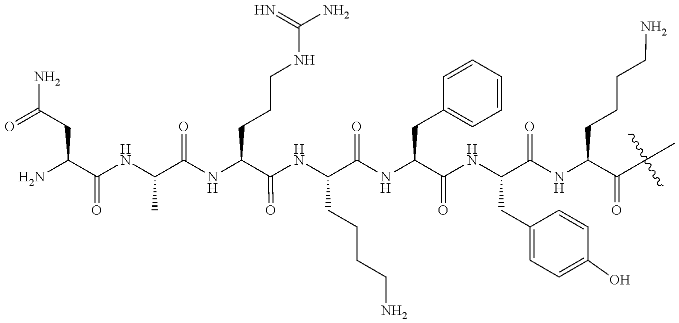 [SMILES: *C(=O)[C@H](CCCCN)NC(=O)[C@H](Cc1ccc(O)cc1)NC(=O)[C@H](Cc1ccccc1)NC(=O)[C@H](CCCCN)NC(=O)[C@H](CCCNC(=N)N)NC(=O)[C@H](C)NC(=O)[C@@H](N)CC(N)=O] | A-9 |
| 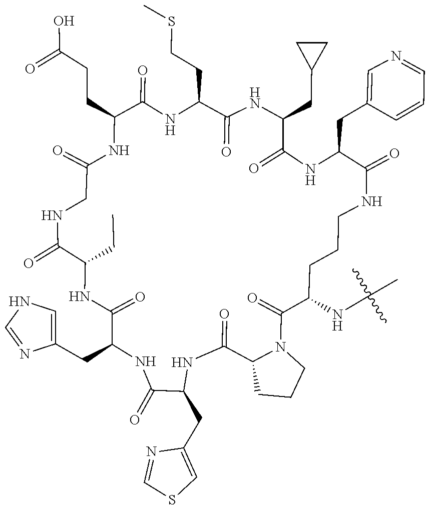 [SMILES: *N[C@H]1CCCNC(=O)[C@H](Cc2cccnc2)NC(=O)[C@H](CC2CC2)NC(=O)[C@H](CCSC)NC(=O)[C@H](CCC(=O)O)NC(=O)CNC(=O)[C@H](CC)NC(=O)[C@H](Cc2c[nH]cn2)NC(=O)[C@H](Cc2cscn2)NC(=O)[C@H]2CCCN2C1=O] | A-10 |
| 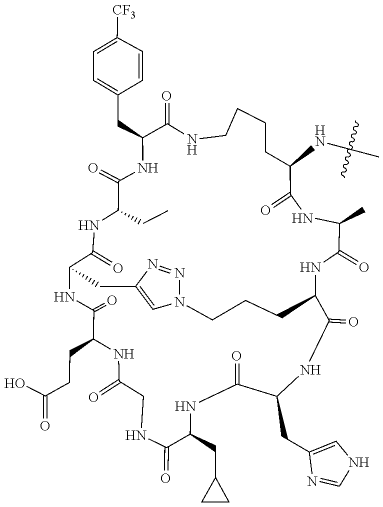 [SMILES: *N[C@@H]1CCCCNC(=O)[C@H](Cc2ccc(C(F)(F)F)cc2)NC(=O)[C@H](CC)NC(=O)[C@H]2Cc3cn(nn3)CCC[C@@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-11 |
| 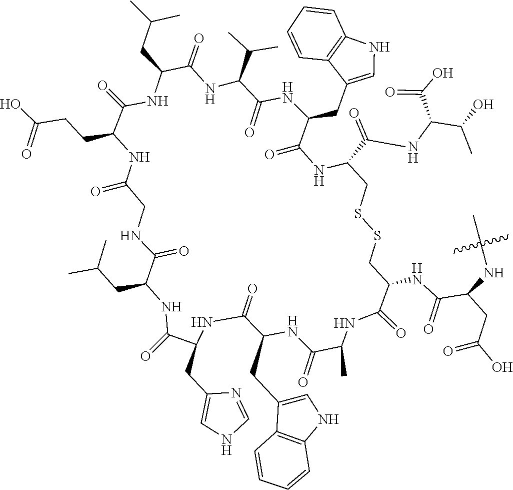 [SMILES: *N[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-12 |
| 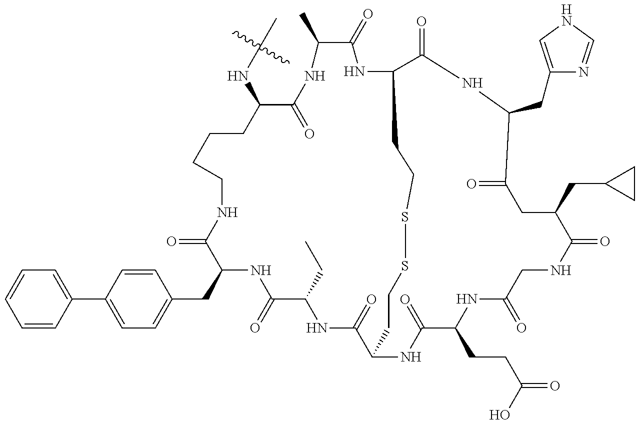 [SMILES: *N[C@@H]1CCCCNC(=O)[C@H](Cc2ccc(-c3ccccc3)cc2)NC(=O)[C@H](CC)NC(=O)[C@H]2CCSSCC[C@@H](NC(=O)[C@H](C)NC1=O)C(=O)N[C@@H](Cc1c[nH]cn1)C(=O)C[C@@H](CC1CC1)C(=O)NCC(=O)N[C@@H](CCC(=O)O)C(=O)N2] | A-13 |
| 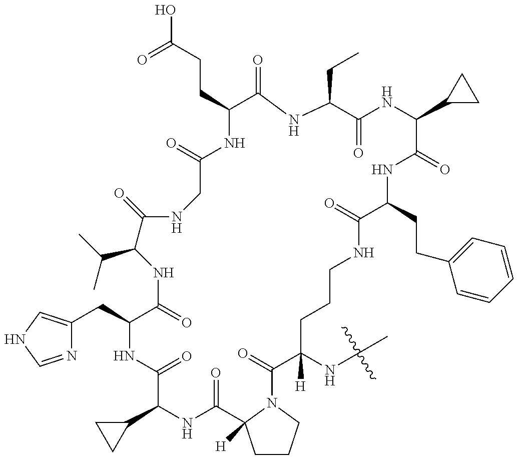 [SMILES: *N[C@H]1CCCNC(=O)[C@H](CCc2ccccc2)NC(=O)[C@H](C2CC2)NC(=O)[C@H](CC)NC(=O)[C@H](CCC(=O)O)NC(=O)CNC(=O)[C@H](C(C)C)NC(=O)[C@H](Cc2c[nH]cn2)NC(=O)[C@H](C2CC2)NC(=O)[C@H]2CCCN2C1=O] | A-14 |
| 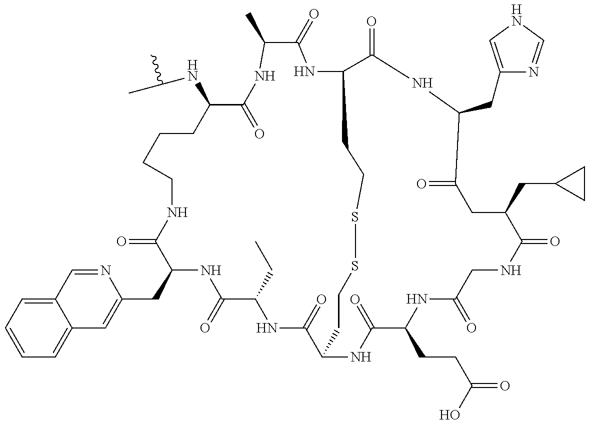 | A-15 |
| 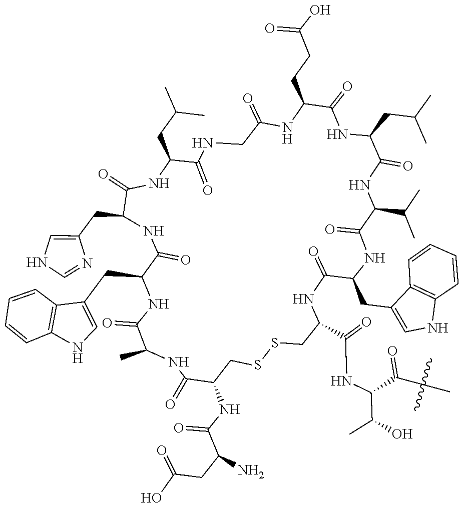 [SMILES: *C(=O)[C@@H](NC(=O)[C@@H]1CSSC[C@H](NC(=O)[C@@H](N)CC(=O)O)C(=O)N[C@@H](C)C(=O)N[C@@H](Cc2c[nH]c3ccccc23)C(=O)N[C@@H](Cc2c[nH]cn2)C(=O)N[C@@H](CC(C)C)C(=O)NCC(=O)N[C@@H](CCC(=O)O)C(=O)N[C@@H](CC(C)C)C(=O)N[C@@H](C(C)C)C(=O)N[C@@H](Cc2c[nH]c3ccccc23)C(=O)N1)[C@@H](C)O] | A-16 |
| 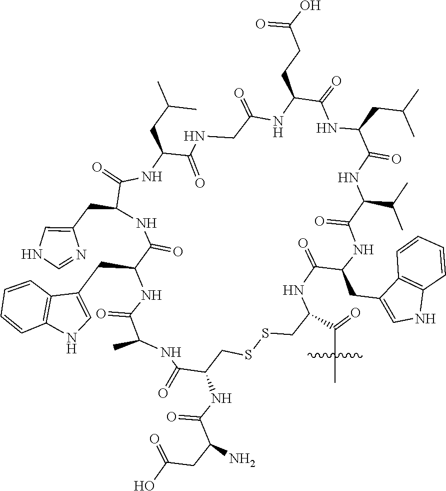 [SMILES: *C(=O)[C@@H]1CSSC[C@H](NC(=O)[C@@H](N)CC(=O)O)C(=O)N[C@@H](C)C(=O)N[C@@H](Cc2c[nH]c3ccccc23)C(=O)N[C@@H](Cc2c[nH]cn2)C(=O)N[C@@H](CC(C)C)C(=O)NCC(=O)N[C@@H](CCC(=O)O)C(=O)N[C@@H](CC(C)C)C(=O)N[C@@H](C(C)C)C(=O)N[C@@H](Cc2c[nH]c3ccccc23)C(=O)N1] | A-17 |
| 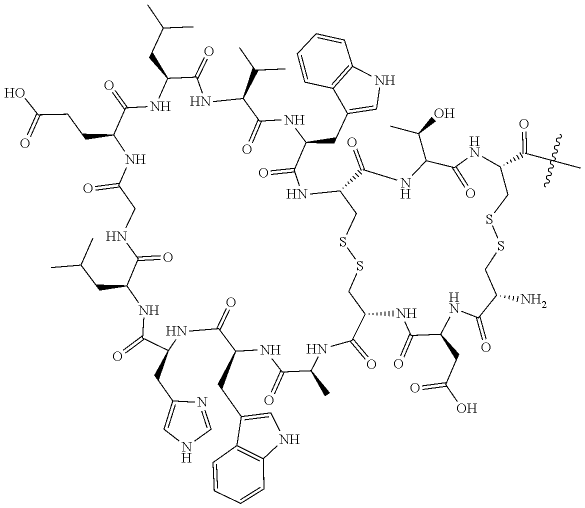 [SMILES: *C(=O)[C@@H]1CSSC[C@H](N)C(=O)N[C@@H](CC(=O)O)C(=O)N[C@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc3c[nH]cn3)NC(=O)[C@H](Cc3c[nH]c4ccccc34)NC(=O)[C@H](C)NC2=O)C(=O)NC([C@@H](C)O)C(=O)N1] | A-18 |
| 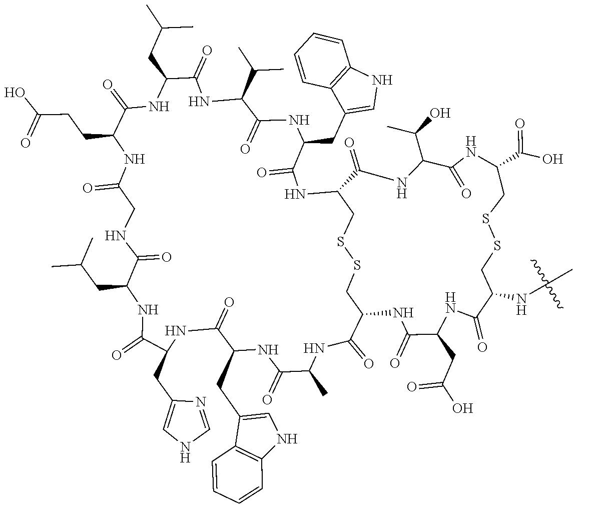 | A-19 |
| 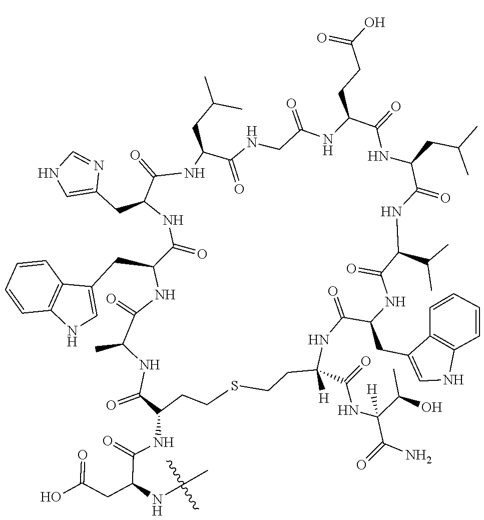 | A-20 |
| 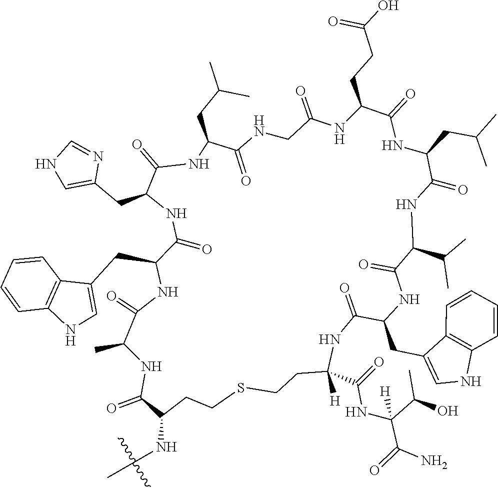 | A-21 |
| 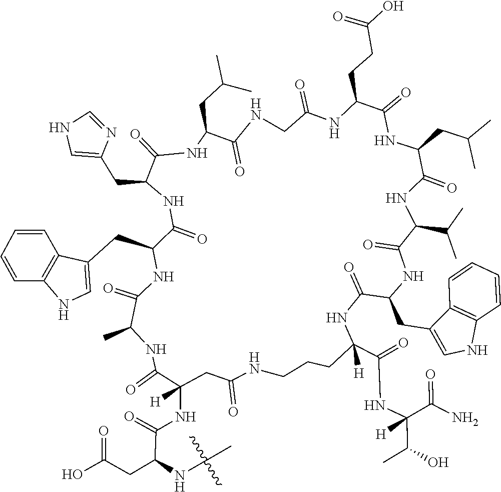 [SMILES: *N[C@@H](CC(=O)O)C(=O)N[C@H]1CC(=O)NCCC[C@@H](C(=O)N[C@H](C(N)=O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-22 |
| 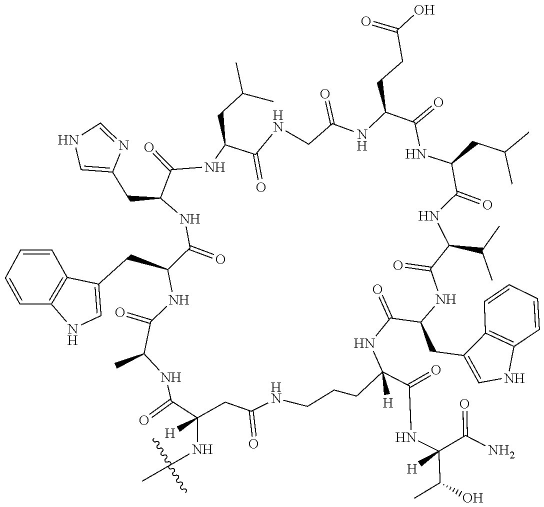 [SMILES: *N[C@H]1CC(=O)NCCC[C@@H](C(=O)N[C@H](C(N)=O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-23 |
| 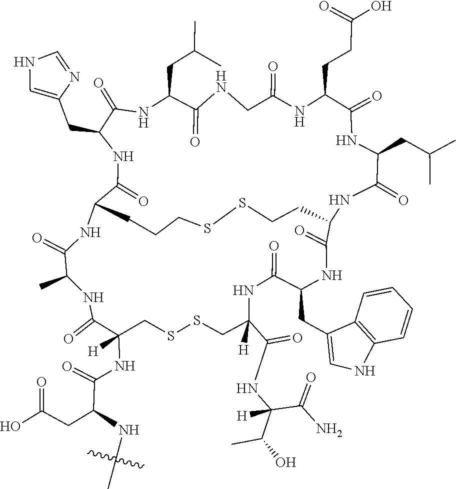 [SMILES: *N[C@@H](CC(=O)O)C(=O)N[C@H]1CSSC[C@@H](C(=O)N[C@H](C(N)=O)[C@@H](C)O)NC(=O)[C@H](Cc2c[nH]c3ccccc23)NC(=O)[C@@H]2CCSSCCC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CC(C)C)C(=O)N2] | A-24 |
| 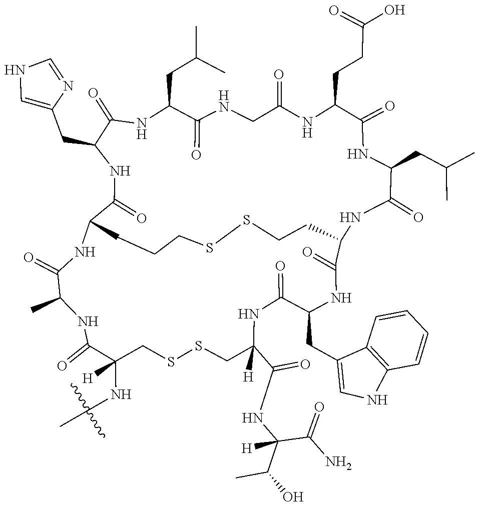 [SMILES: *N[C@H]1CSSC[C@@H](C(=O)N[C@H](C(N)=O)[C@@H](C)O)NC(=O)[C@H](Cc2c[nH]c3ccccc23)NC(=O)C[C@@H]2CCSSCCC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CC(C)C)C(=O)N2] | A-25 |
| 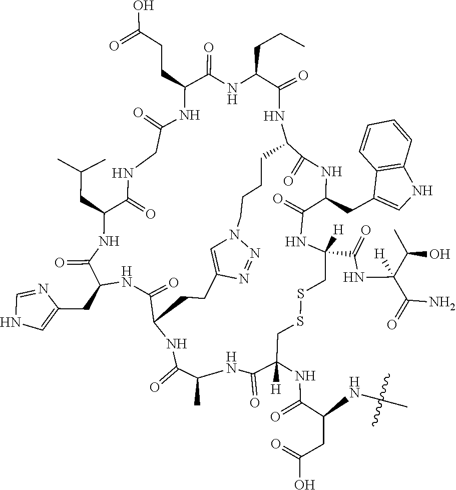 [SMILES: *N[C@@H](CC(=O)O)C(=O)N[C@H]1CSSC[C@@H](C(=O)N[C@H](C(N)=O)[C@@H](C)O)NC(=O)[C@H](Cc2c[nH]c3ccccc23)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CCC)C(=O)N2] | A-26 |
| 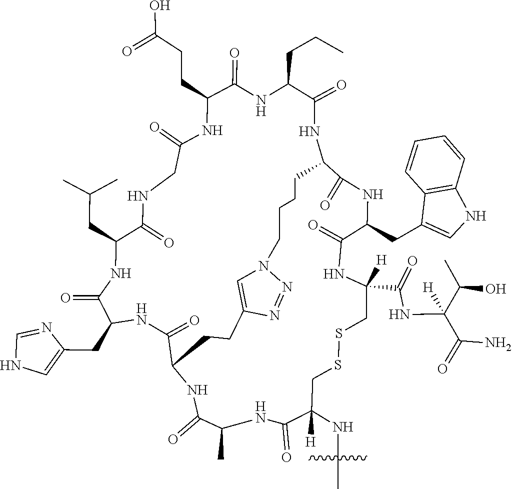 [SMILES: *N[C@H]1CSSC[C@@H](C(=O)N[C@H](C(N)=O)[C@@H](C)O)NC(=O)[C@H](Cc2c[nH]c3ccccc23)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC(C)C)C(=O)NCC(=O)N[C@@H](CCC(=O)O)C(=O)N[C@@H](CCC)C(=O)N2] | A-27 |
| 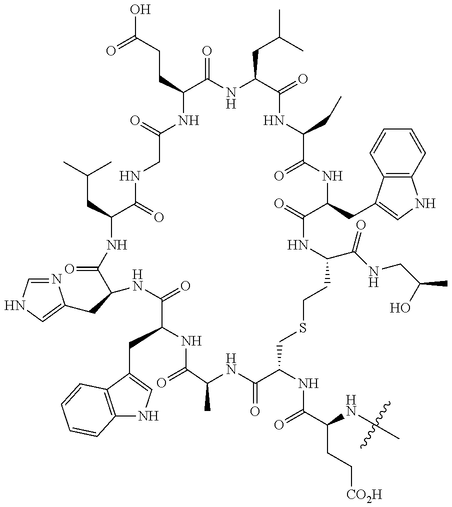 [SMILES: *N[C@@H](CCC(=O)O)C(=O)N[C@H]1CSCC[C@@H](C(=O)NC[C@@H](C)O)NC(=O)[C@H](Cc2c[nH]c3ccccc23)NC(=O)[C@H](CC)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-28 |
| 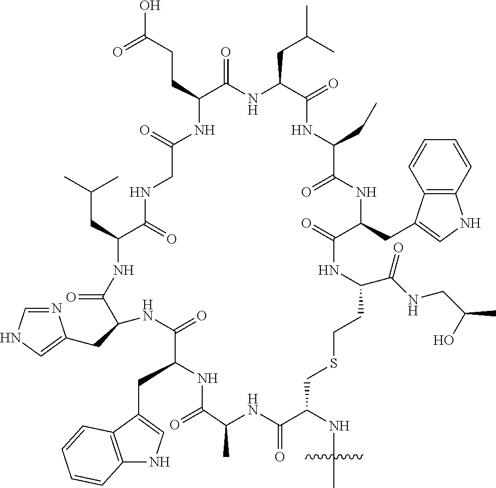 [SMILES: *N[C@H]1CSCC[C@@H](C(=O)NC[C@@H](C)O)NC(=O)[C@H](Cc2c[nH]c3ccccc23)NC(=O)[C@H](CC)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-29 |
| 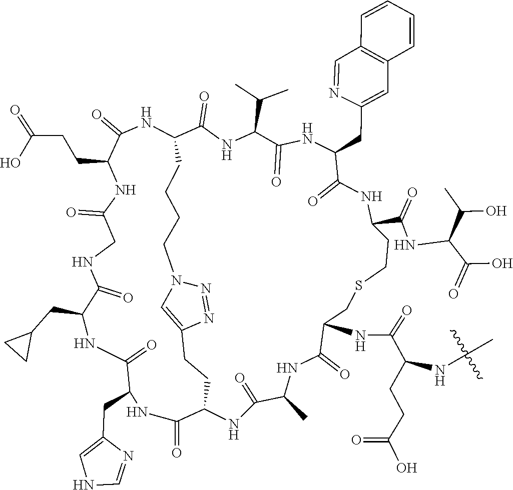 [SMILES: *N[C@@H](CCC(=O)O)C(=O)N[C@H]1CSCC[C@@H](C(=O)N[C@H](C(=O)O)C(C)O)NC(=O)[C@H](Cc2cc3ccccc3cn2)NC(=O)[C@H](C(C)C)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-30 |
| 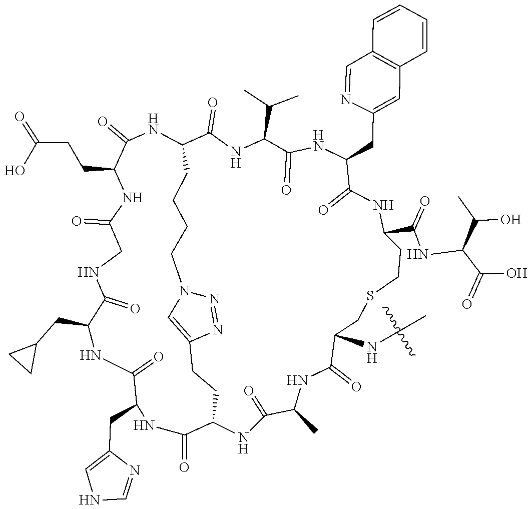 [SMILES: *N[C@H]1CSCC[C@@H](C(=O)N[C@H](C(=O)O)C(C)O)NC(=O)[C@H](Cc2cc3ccccc3cn2)NC(=O)[C@H](C(C)C)NC(=O)[C@@H]2CCCCn3cc(nn3)CC[C@H](NC(=O)[C@H](C)NC1=O)C(=O)N[C@@H](Cc1c[nH]cn1)C(=O)N[C@@H](CC1CC1)C(=O)NCC(=O)N[C@@H](CCC(=O)O)C(=O)N2] | A-31 |
| 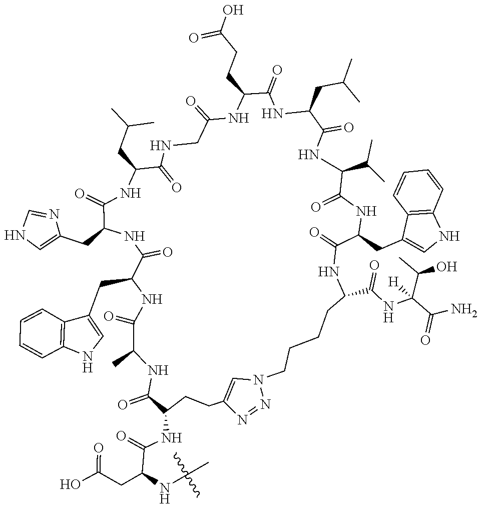 | A-32 |
| 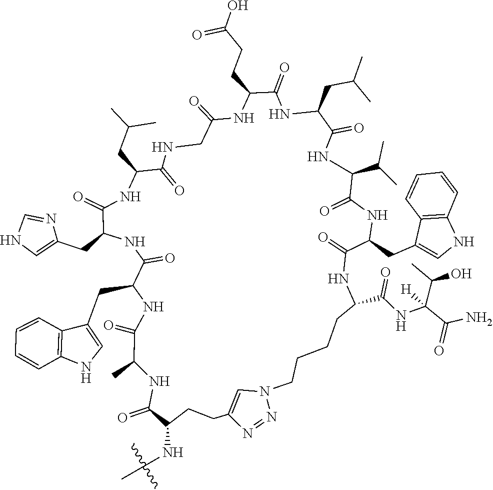 | A-33 |
| 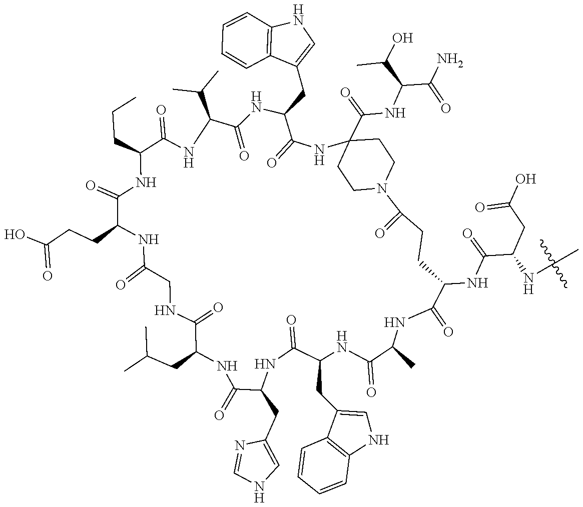 [SMILES: *N[C@@H](CC(=O)O)C(=O)N[C@H]1CCC(=O)N2CCC(C(=O)N[C@H](C(N)=O)C(C)O)(CC2)NC(=O)[C@H](Cc2c[nH]c3ccccc23)NC(=O)[C@H](C(C)C)NC(=O)[C@H](CCC)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-34 |
| 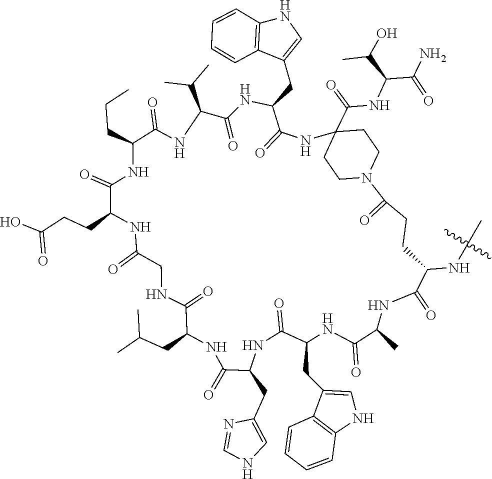 [SMILES: *N[C@H]1CCC(=O)N2CCC(C(=O)N[C@H](C(N)=O)C(C)O)(CC2)NC(=O)[C@H](Cc2c[nH]c3ccccc23)NC(=O)[C@H](C(C)C)NC(=O)[C@H](CCC)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-35 |
| 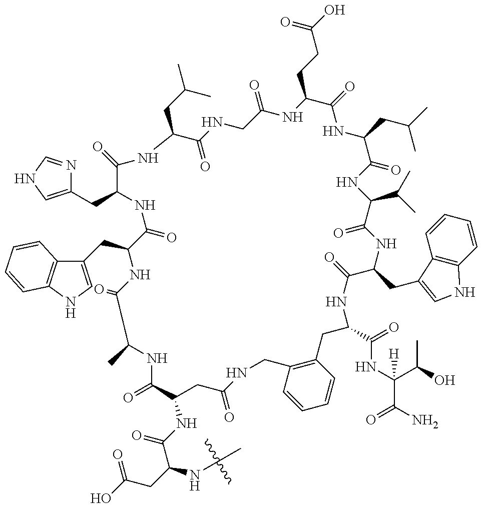 | A-36 |
| 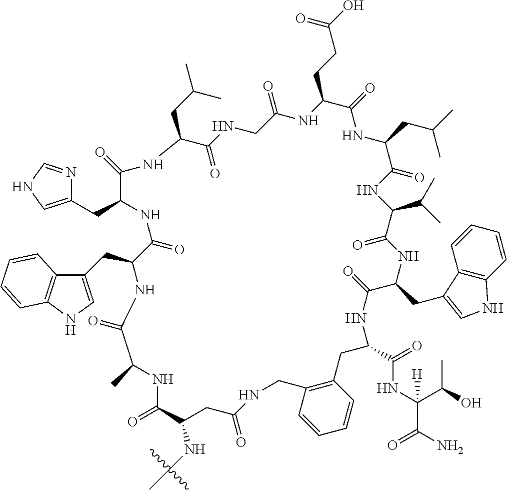 | A-37 |
| 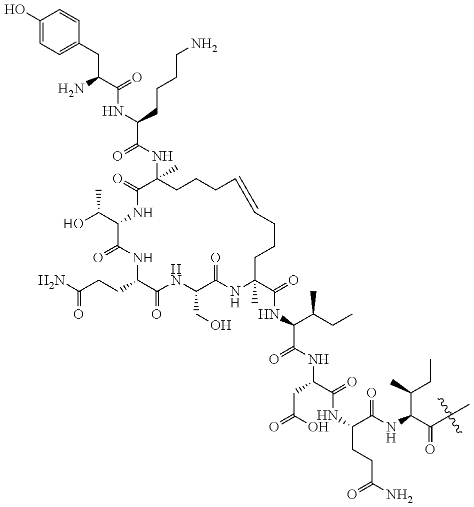 [SMILES: *C(=O)[C@@H](NC(=O)[C@H](CCC(N)=O)NC(=O)[C@H](CC(=O)O)NC(=O)[C@@H](NC(=O)[C@]1(C)CCC/C=C/CCC[C@](C)(NC(=O)[C@H](CCCCN)NC(=O)[C@@H](N)Cc2ccc(O)cc2)C(=O)N[C@@H]([C@@H](C)O)C(=O)N[C@@H](CCC(N)=O)C(=O)N[C@@H](CO)C(=O)N1)[C@@H](C)CC)[C@@H](C)CC] | A-38 |
| 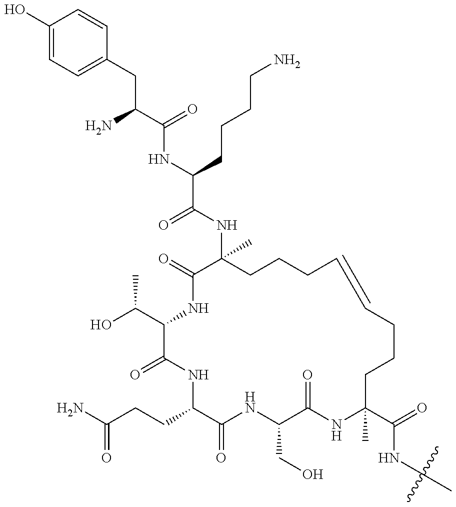 [SMILES: *NC(=O)[C@]1(C)CCC/C=C/CCC[C@](C)(NC(=O)[C@H](CCCCN)NC(=O)[C@@H](N)Cc2ccc(O)cc2)C(=O)N[C@@H]([C@@H](C)O)C(=O)N[C@@H](CCC(N)=O)C(=O)N[C@@H](CO)C(=O)N1] | A-39 |
| 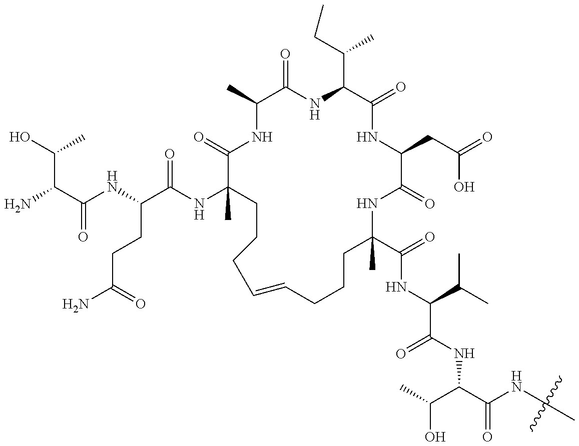 [SMILES: *NC(=O)[C@@H](NC(=O)[C@@H](NC(=O)[C@]1(C)CCC/C=C/CCC[C@](C)(NC(=O)[C@H](CCC(N)=O)NC(=O)[C@H](N)[C@@H](C)O)C(=O)N[C@@H](C)C(=O)N[C@@H]([C@@H](C)CC)C(=O)N[C@@H](CC(=O)O)C(=O)N1)C(C)C)[C@@H](C)O] | A-40 |
| 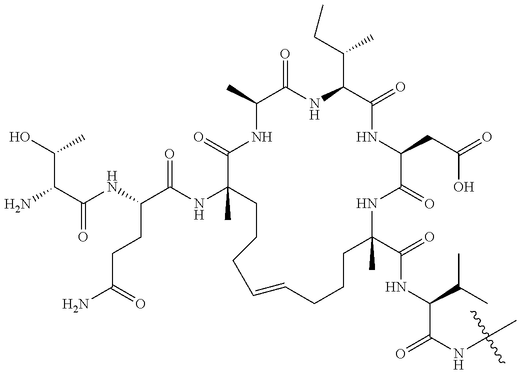 [SMILES: *NC(=O)[C@@H](NC(=O)[C@]1(C)CCC/C=C/CCC[C@](C)(NC(=O)[C@H](CCC(N)=O)NC(=O)[C@H](N)[C@@H](C)O)C(=O)N[C@@H](C)C(=O)N[C@@H]([C@@H](C)CC)C(=O)N[C@@H](CC(=O)O)C(=O)N1)C(C)C] | A-41 |
| 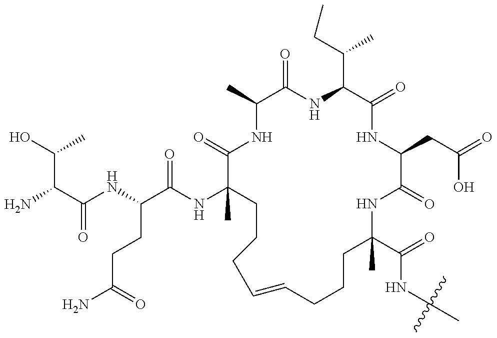 [SMILES: *NC(=O)[C@]1(C)CCC/C=C/CCC[C@](C)(NC(=O)[C@H](CCC(N)=O)NC(=O)[C@H](N)[C@@H](C)O)C(=O)N[C@@H](C)C(=O)N[C@@H]([C@@H](C)CC)C(=O)N[C@@H](CC(=O)O)C(=O)N1] | A-42 |
| 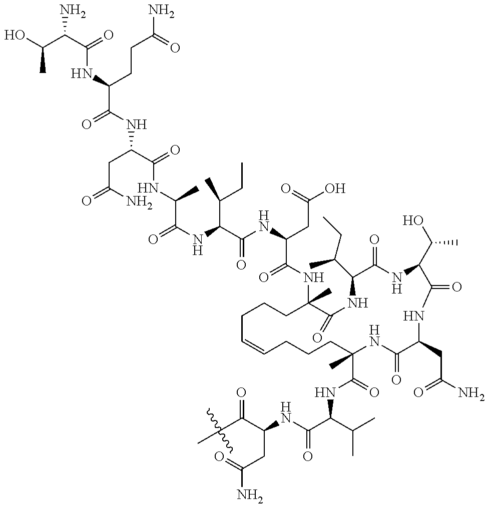 [SMILES: *C(=O)[C@H](CC(N)=O)NC(=O)[C@@H](NC(=O)[C@]1(C)CCC/C=C\CCC[C@](C)(NC(=O)[C@H](CC(=O)O)NC(=O)[C@@H](NC(=O)[C@H](C)NC(=O)[C@H](CC(N)=O)NC(=O)[C@H](CCC(N)=O)NC(=O)[C@@H](N)[C@@H](C)O)[C@@H](C)CC)C(=O)N[C@@H]([C@@H](C)CC)C(=O)N[C@@H]([C@@H](C)O)C(=O)N[C@@H](CC(N)=O)C(=O)N1)C(C)C] | A-43 |
| 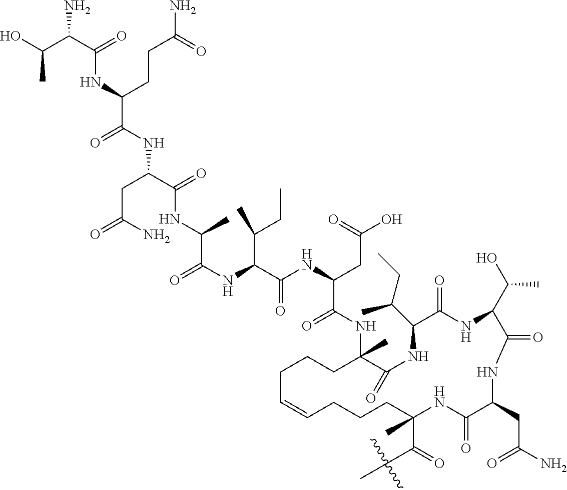 [SMILES: *C(=O)[C@]1(C)CCC/C=C\CCC[C@](C)(NC(=O)[C@H](CC(=O)O)NC(=O)[C@@H](NC(=O)[C@H](C)NC(=O)[C@H](CC(N)=O)NC(=O)[C@H](CCC(N)=O)NC(=O)[C@@H](N)[C@@H](C)O)[C@@H](C)CC)C(=O)N[C@@H]([C@@H](C)CC)C(=O)N[C@@H]([C@@H](C)O)C(=O)N[C@@H](CC(N)=O)C(=O)N1] | A-44 |
| 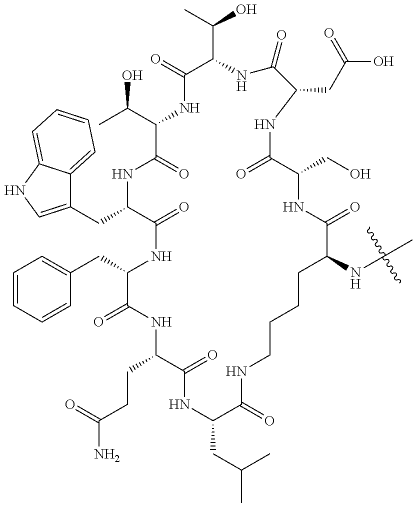 [SMILES: *N[C@H]1CCCCNC(=O)[C@H](CC(C)C)NC(=O)[C@H](CCC(N)=O)NC(=O)[C@H](Cc2ccccc2)NC(=O)[C@H](Cc2c[nH]c3ccccc23)NC(=O)[C@H]([C@@H](C)O)NC(=O)[C@H]([C@@H](C)O)NC(=O)[C@H](CC(=O)O)NC(=O)[C@H](CO)NC1=O] | A-45 |
| 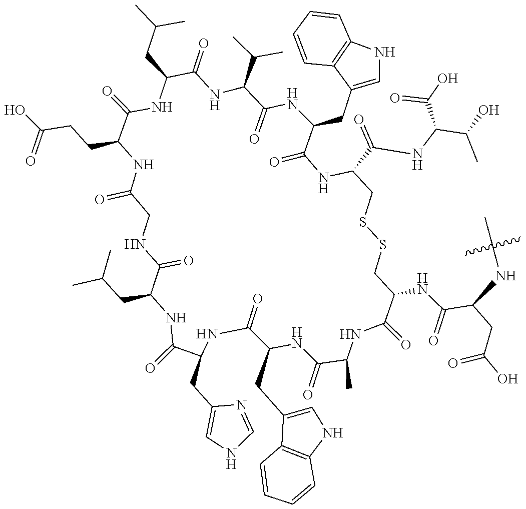 [SMILES: *N[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-46 |
| 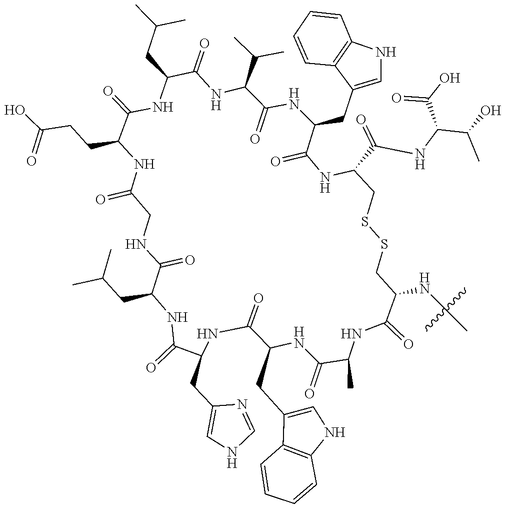 [SMILES: *N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2c[nH]cn2)NC(=O)[C@H](Cc2c[nH]c3ccccc23)NC(=O)[C@H](C)NC1=O] | A-47 |
| 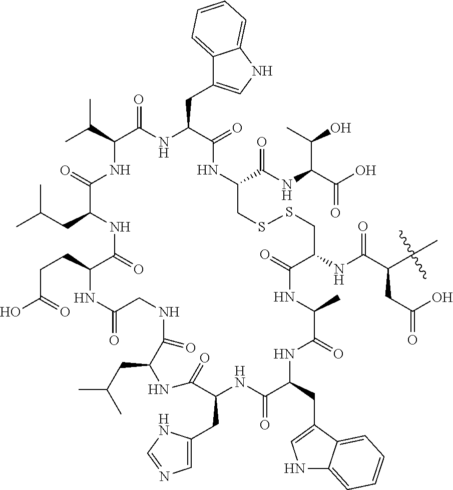 [SMILES: *[C@@H](CC(=O)O)C(=O)N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2cnc[nH]2)NC(=O)[C@H](Cc2c[nH]c3ccccc23)NC(=O)[C@H](C)NC1=O] | A-48 |
| 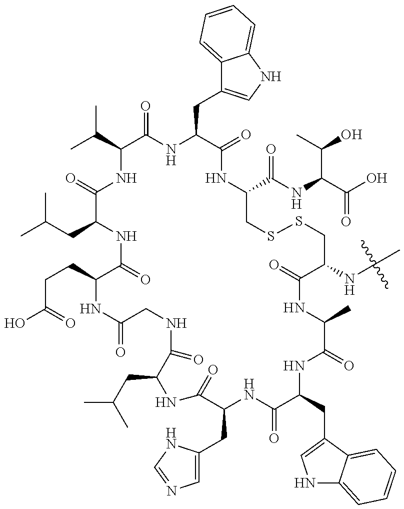 [SMILES: *N[C@H]1CSSC[C@@H](C(=O)N[C@H](C(=O)O)[C@@H](C)O)NC(=O)[C@H](Cc2c[nH]c3ccccc23)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc2cnc[nH]2)NC(=O)[C@H](Cc2c[nH]c3ccccc23)NC(=O)[C@H](C)NC1=O] | A-49 |
| 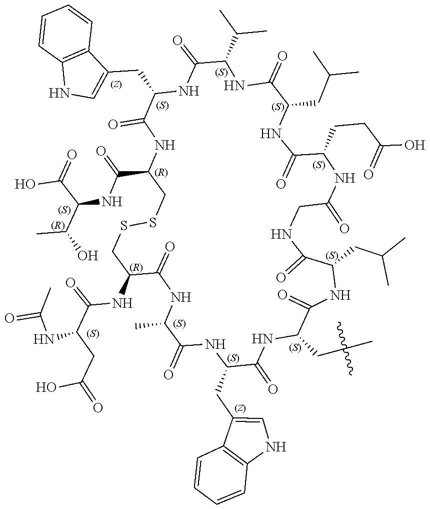 | A-50 |

In some embodiments, an antibody binding moiety is an antibody binding moiety described herein. In some embodiments, a protein binding moiety is an antibody binding moiety described herein. In some embodiments, —COOH and/or amino groups of amino acid residues, e.g., those at the C-terminus or N-terminus, is optionally capped. For example, in some embodiments, a —COOH group (e.g., a C-terminus —COOH) is amidated (e.g., converted into —CON(R′)₂, e.g., —C(O)NHR (e.g., —C(O)NH₂)), and in some embodiments, an amino group, e.g. —NH₂ (e.g., a N-terminus —NH₂) is capped with R′— or R′C(O)— (e.g., in some embodiments, by conversion —NH₂ into —NHR′ (e.g., —NHC(O)R, (e.g., —NHC(O)CH₃))).

In some embodiments, an antibody binding moiety is or comprises optionally substituted A-1. In some embodiments, an antibody binding moiety is or comprises optionally substituted A-2. In some embodiments, an antibody binding moiety is or comprises optionally substituted A-3. In some embodiments, an antibody binding moiety is or comprises optionally substituted A-4. In some embodiments, an antibody binding moiety is or comprises optionally substituted A-5. In some embodiments, an antibody binding moiety is or comprises optionally substituted A-6. In some embodiments, an antibody binding moiety is or comprises optionally substituted A-7. In some embodiments, an antibody binding moiety is or comprises optionally substituted A-8. In some embodiments, an antibody binding moiety is or comprises optionally substituted A-9. In some embodiments, an antibody binding moiety is or comprises optionally substituted A-10. In some embodiments, an antibody binding moiety is or comprises optionally substituted A-11. In some embodiments, an antibody binding moiety is or comprises optionally substituted A-12. In some embodiments, an antibody binding moiety is or comprises optionally substituted A-13. In some embodiments, an antibody binding moiety is or comprises optionally substituted A-14. In some embodiments, an antibody binding moiety is or comprises optionally substituted A-15. In some embodiments, an antibody binding moiety is or comprises optionally substituted A-16. In some embodiments, an antibody binding moiety is or comprises optionally substituted A-17. In some embodiments, an antibody binding moiety is or comprises optionally substituted A-18. In some embodiments, an antibody binding moiety is or comprises optionally substituted A-19. In some embodiments, an antibody binding moiety is or comprises optionally substituted A-20. In some embodiments, an antibody binding moiety is or comprises optionally substituted A-21. In some embodiments, an antibody binding moiety is or comprises optionally substituted A-22. In some embodiments, an antibody binding moiety is or comprises optionally substituted A-23. In some embodiments, an antibody binding moiety is or comprises optionally substituted A-24. In some embodiments, an antibody binding moiety is or comprises optionally substituted A-25. In some embodiments, an antibody binding moiety is or comprises optionally substituted A-26. In some embodiments, an antibody binding moiety is or comprises optionally substituted A-27. In some embodiments, an antibody binding moiety is or comprises optionally substituted A-28. In some embodiments, an antibody binding moiety is or comprises optionally substituted A-29. In some embodiments, an antibody binding moiety is or comprises optionally substituted A-30. In some embodiments, an antibody binding moiety is or comprises optionally substituted A-31. In some embodiments, an antibody binding moiety is or comprises optionally substituted A-32. In some embodiments, an antibody binding moiety is or comprises optionally substituted A-33. In some embodiments, an antibody binding moiety is or comprises optionally substituted A-34. In some embodiments, an antibody binding moiety is or comprises optionally substituted A-35. In some embodiments, an antibody binding moiety is or comprises optionally substituted A-36. In some embodiments, an antibody binding moiety is or comprises optionally substituted A-37. In some embodiments, an antibody binding moiety is or comprises optionally substituted A-38. In some embodiments, an antibody binding moiety is or comprises optionally substituted A-39. In some embodiments, an antibody binding moiety is or comprises optionally substituted A-40. In some embodiments, an antibody binding moiety is or comprises optionally substituted A-41. In some embodiments, an antibody binding moiety is or comprises optionally substituted A-42. In some embodiments, an antibody binding moiety is or comprises optionally substituted A-43. In some embodiments, an antibody binding moiety is or comprises optionally substituted A-44. In some embodiments, an antibody binding moiety is or comprises optionally substituted A-45. In some embodiments, an antibody binding moiety is or comprises optionally substituted A-46. In some embodiments, an antibody binding moiety is or comprises optionally substituted A-47. In some embodiments, an antibody binding moiety is or comprises optionally substituted A-48. In some embodiments, an antibody binding moiety is or comprises optionally substituted A-49. In some embodiments, an antibody binding moiety is or comprises optionally substituted A-50. In some embodiments, such an antibody binding moiety is an antibody binding moiety. In some embodiments, such an antibody binding moiety is a universal antibody binding moiety.

In some embodiments, an antibody binding moiety is A-1. In some embodiments, an antibody binding moiety is A-2. In some embodiments, an antibody binding moiety is A-3. In some embodiments, an antibody binding moiety is A-4. In some embodiments, an antibody binding moiety is A-5. In some embodiments, an antibody binding moiety is A-6. In some embodiments, an antibody binding moiety is A-7. In some embodiments, an antibody binding moiety is A-8. In some embodiments, an antibody binding moiety is A-9. In some embodiments, an antibody binding moiety is A-10. In some embodiments, an antibody binding moiety is A-11. In some embodiments, an antibody binding moiety is A-12. In some embodiments, an antibody binding moiety is A-13. In some embodiments, an antibody binding moiety is A-14. In some embodiments, an antibody binding moiety is A-15. In some embodiments, an antibody binding moiety is A-16. In some embodiments, an antibody binding moiety is A-17. In some embodiments, an antibody binding moiety is A-18. In some embodiments, an antibody binding moiety is A-19. In some embodiments, an antibody binding moiety is A-20. In some embodiments, an antibody binding moiety is A-21. In some embodiments, an antibody binding moiety is A-22. In some embodiments, an antibody binding moiety is A-23. In some embodiments, an antibody binding moiety is A-24. In some embodiments, an antibody binding moiety is A-25. In some embodiments, an antibody binding moiety is A-26. In some embodiments, an antibody binding moiety is A-27. In some embodiments, an antibody binding moiety is A-28. In some embodiments, an antibody binding moiety is A-29. In some embodiments, an antibody binding moiety is A-30. In some embodiments, an antibody binding moiety is A-31. In some embodiments, an antibody binding moiety is A-32. In some embodiments, an antibody binding moiety is A-33. In some embodiments, an antibody binding moiety is A-34. In some embodiments, an antibody binding moiety is A-35. In some embodiments, an antibody binding moiety is A-36. In some embodiments, an antibody binding moiety is A-37. In some embodiments, an antibody binding moiety is A-38. In some embodiments, an antibody binding moiety is A-39. In some embodiments, an antibody binding moiety is A-40. In some embodiments, an antibody binding moiety is A-41. In some embodiments, an antibody binding moiety is A-42. In some embodiments, an antibody binding moiety is A-43. In some embodiments, an antibody binding moiety is A-44. In some embodiments, an antibody binding moiety is A-45. In some embodiments, an antibody binding moiety is A-46. In some embodiments, an antibody binding moiety is A-47. In some embodiments, an antibody binding moiety is A-48. In some embodiments, an antibody binding moiety is A-49. In some embodiments, such an antibody binding moiety is an antibody binding moiety. In some embodiments, such an antibody binding moiety is a universal antibody binding moiety.

In some embodiments, an antibody binding moiety, e.g., a protein binding moiety (e.g., an antibody binding moiety (e.g., a universal antibody binding moiety)) comprises a peptide unit, and is connected to a linker moiety through the C-terminus of the peptide unit. In some embodiments, it is connected to a linker moiety through the N-terminus of the peptide unit. In some embodiments, it is connected to a linker through a side chain group of the peptide unit. In some embodiments, an antibody binding moiety, e.g., a universal antibody binding moiety comprises a peptide unit, and is connected to an antibody binding moiety optionally through a linker moiety through the C-terminus of the peptide unit. In some embodiments, an antibody binding moiety, e.g., a protein binding moiety (e.g., an antibody binding moiety (e.g., a universal antibody binding moiety)) comprises a peptide unit, and is connected to an antibody binding moiety optionally through a linker moiety through the N-terminus of the peptide unit. In some embodiments, In some embodiments, an antibody binding moiety, e.g., a protein binding moiety (e.g., an antibody binding moiety (e.g., a universal antibody binding moiety)) comprises a peptide unit, and is connected to an antibody binding moiety optionally through a linker moiety through a side chain of the peptide unit.

In some embodiments, an antibody binding moiety is or comprises (DCAWHLGELVWCT)-, (SEQ ID NO:35), wherein 1-5 (e.g., 1, 2, 3, 4, or 5) amino acid residues may be independently and optionally replaced with another amino acid residue, 1-5 (e.g., 1, 2, 3, 4, or 5) amino acid residues may be independently and optionally deleted, and/or 1-5 (e.g., 1, 2, 3, 4, or 5) amino acid residues may be independently and optionally inserted. In some embodiments, it is connected to the rest of a molecule through its N-terminus. In some embodiments, it is connected to the rest of a molecule through its C-terminus. In some embodiments, it is connected to the rest of a molecule through a side chain of an amino acid residue (e.g., various X residues as described in the present disclosure). In some embodiments, two cysteine residues form a disulfide bond. In some embodiments, an antibody binding moiety is or comprises

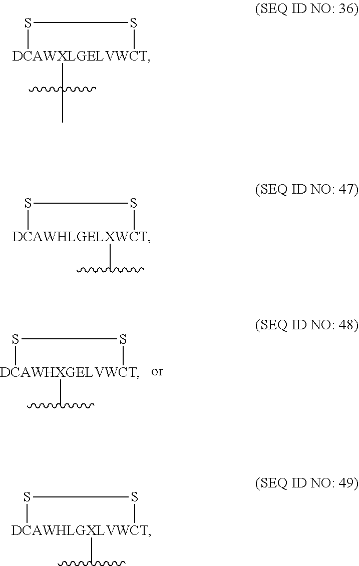

wherein X is an amino acid residue bonded to the rest of a compound or agent, and wherein 1-5 (e.g., 1, 2, 3, 4, or 5) amino acid residues may be independently and optionally replaced with another amino acid residue, 1-5 (e.g., 1, 2, 3, 4, or 5) amino acid residues may be independently and optionally deleted, and/or 1-5 (e.g., 1, 2, 3, 4, or 5) amino acid residues may be independently and optionally inserted. In some embodiments, the total number of replacements, deletions and insertions is no more than 10 (e.g., 0, or no more than 1, 2, 3, 4, 5, 6, 7, 8, 9, or 10). In some embodiments, the total number is 0. In some embodiments, the total number is no more than 1. In some embodiments, the total number is no more than 2. In some embodiments, the total number is no more than 3. In some embodiments, the total number is no more than 4. In some embodiments, the total number is no more than 5. In some embodiments, the total number is no more than 6. In some embodiments, the total number is no more than 7. In some embodiments, the total number is no more than 8. In some embodiments, the total number is no more than 9. In some embodiments, the total number is no more than 10. In some embodiments, there are no insertions. In some embodiments, there are no deletions. In some embodiments, there are no replacements. In some embodiments, an antibody binding moiety is or comprises

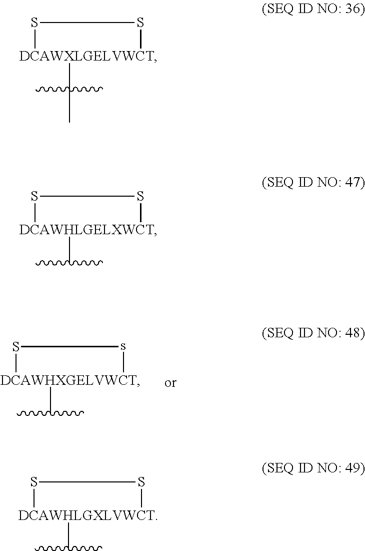

wherein X is an amino acid residue bonded to the rest of a compound or agent. In some embodiments, X is —N(R′)—CH(−)—C(O)—. In some embodiments, X is —N(R′)—CH(-L^{LG1}-)-C(O)—. In some embodiments, X is —N(R′)—CH(-L^{LG1}-L^{LG2}-)-C(O)—. In some embodiments, X is —N(R′)—CH(-L^{LG1}-L^{LG2}-L^{LG3}-)-C(O)—. In some embodiments, X is —N(R′)—CH(-L^{LG1}-L^{LG2}-L^{LG3}-L^{LG4}-)-C(O)—. In some embodiments, an antibody binding moiety is or comprises

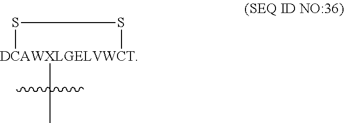

In some embodiments, an antibody binding moiety is or comprises

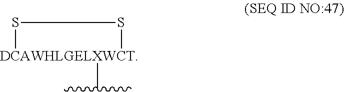

In some embodiments, an antibody binding moiety is or comprises

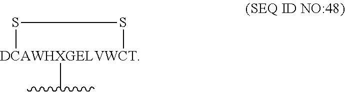

In some embodiments, an antibody binding moiety is or comprises

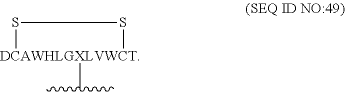

In some embodiments, X is a residue of

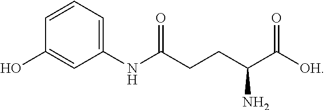 [SMILES: N[C@@H](CCC(=O)Nc1cccc(O)c1)C(=O)O]

In some embodiments, X is a residue of

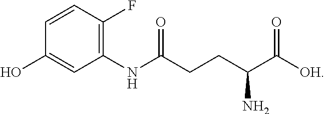 [SMILES: N[C@@H](CCC(=O)Nc1cc(O)ccc1F)C(=O)O]

In some embodiments, X is a residue of

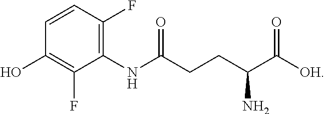 [SMILES: N[C@@H](CCC(=O)Nc1c(F)ccc(O)c1F)C(=O)O]

In some embodiments, X is a residue of

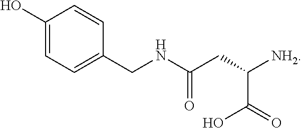 [SMILES: N[C@@H](CC(=O)NCc1ccc(O)cc1)C(=O)O]

In some embodiments, X is a residue of

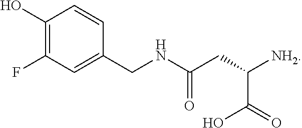 [SMILES: N[C@@H](CC(=O)NCc1ccc(O)c(F)c1)C(=O)O]

In some embodiments, X is a residue of

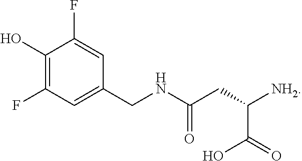 [SMILES: N[C@@H](CC(=O)NCc1cc(F)c(O)c(F)c1)C(=O)O]

In some embodiments, X is a residue of

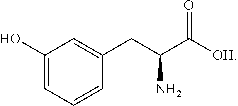 [SMILES: N[C@@H](Cc1cccc(O)c1)C(=O)O]

In some embodiments, X is a residue of

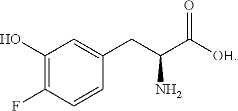 [SMILES: N[C@@H](Cc1ccc(F)c(O)c1)C(=O)O]

In some embodiments, X is a residue of

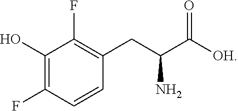 [SMILES: N[C@@H](Cc1ccc(F)c(O)c1F)C(=O)O]

In some embodiments, X is K. In some embodiments, X is D. In some embodiments, X is a residue of Dab. In some embodiments, X is E. In some embodiments, X is a residue of

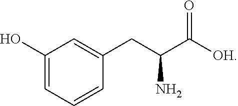 [SMILES: N[C@@H](Cc1cccc(O)c1)C(=O)O]

In some embodiments, the present disclosure provides an amino acid having the structure of

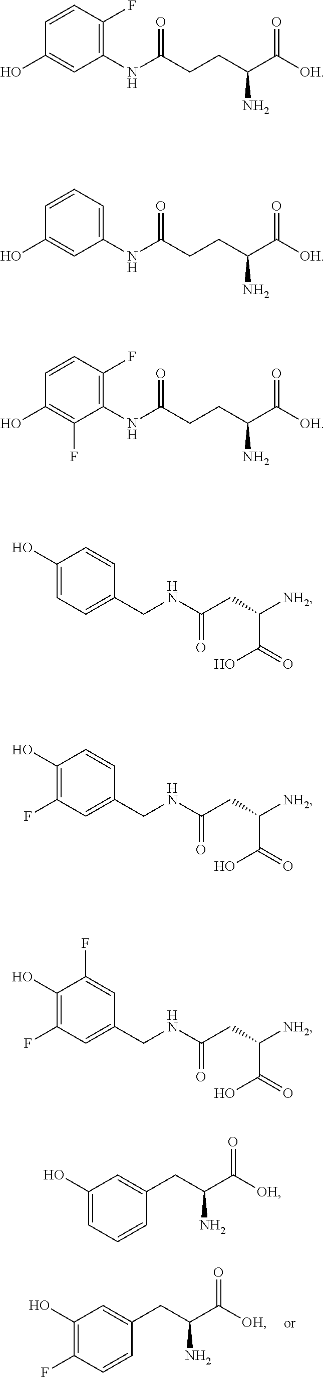

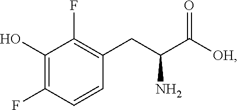 [SMILES: N[C@@H](Cc1ccc(F)c(O)c1F)C(=O)O]

or a salt thereof, or an ester thereof, or an activated ester thereof, or a stereoisomer thereof, or an ester or an activated ester of a stereoisomer. In some embodiments, such antibody binding moieties are antibody binding moieties.

In some embodiments, an antibody binding moiety, e.g., a universal antibody binding moiety, is or comprises a small molecule entity, with a molecular weight of, e.g., less than 10000, 9000, 8000, 7000, 6000, 5000, 4000, 3000, 2000, 1500, 1000, etc. Suitable such antibody binding moieties include small molecule Fc binder moieties, e.g., those described in U.S. Pat. No. 9,745,339, US 201/30131321, etc. In some embodiments, an antibody binding moiety is of such a structure that its corresponding compound is a compound described in U.S. Pat. No. 9,745,339 or US 2013/0131321, the compounds of each of which are independently incorporated herein by reference. In some embodiments, an antibody binding moiety ABT is of such a structure that H-ABT is a compound described in U.S. Pat. No. 9,745,339 or US 2013/0131321, the compounds of each of which are independently incorporated herein by reference. In some embodiments, such a compound can bind to an antibody. In some embodiments, such a compound can bind to Fc region of an antibody.

In some embodiments, an antibody binding moiety is or comprises optionally substituted

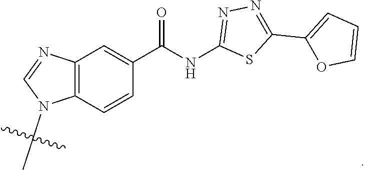 [SMILES: *n1cnc2cc(C(=O)Nc3nnc(-c4ccco4)s3)ccc21]

In some embodiments, an antibody binding moiety is or comprises

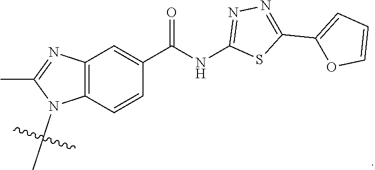 [SMILES: *n1c(C)nc2cc(C(=O)Nc3nnc(-c4ccco4)s3)ccc21]

In some embodiments, an antibody binding moiety is or comprises optionally substituted

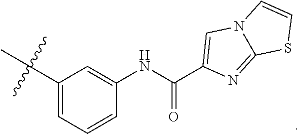 [SMILES: *c1cccc(NC(=O)c2cn3ccsc3n2)c1]

In some embodiments, an antibody binding moiety is or comprises

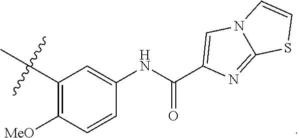 [SMILES: *c1cc(NC(=O)c2cn3ccsc3n2)ccc1OC]

In some embodiments, an antibody binding moiety is or comprises

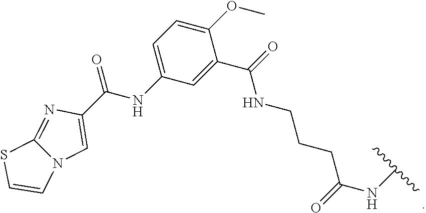 [SMILES: *NC(=O)CCCNC(=O)c1cc(NC(=O)c2cn3ccsc3n2)ccc1OC]

In some embodiments, an antibody binding moiety is or comprises optionally substituted

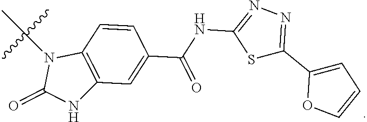 [SMILES: *n1c(=O)[nH]c2cc(C(=O)Nc3nnc(-c4ccco4)s3)ccc21]

In some embodiments, an antibody binding moiety is or comprises

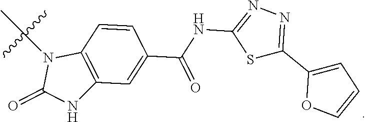 [SMILES: *n1c(=O)[nH]c2cc(C(=O)Nc3nnc(-c4ccco4)s3)ccc21]

In some embodiments, an antibody binding moiety is or comprises optionally substituted

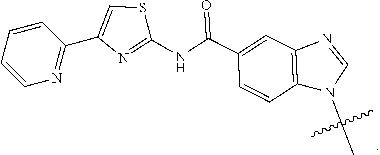 [SMILES: *n1cnc2cc(C(=O)Nc3nc(-c4ccccn4)cs3)ccc21]

In some embodiments, an antibody binding moiety is or comprises

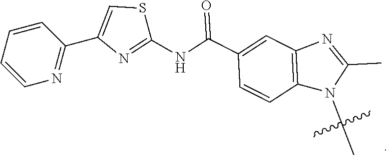 [SMILES: *n1c(C)nc2cc(C(=O)Nc3nc(-c4ccccn4)cs3)ccc21]

In some embodiments, antibody binding moiety is or comprises

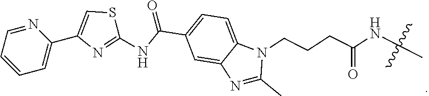 [SMILES: *NC(=O)CCCn1c(C)nc2cc(C(=O)Nc3nc(-c4ccccn4)cs3)ccc21]

In some embodiments, antibody binding moiety is or or comprises

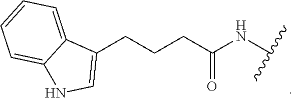 [SMILES: *NC(=O)CCCc1c[nH]c2ccccc12]

In some embodiments, antibody binding moiety is comprises optionally substituted

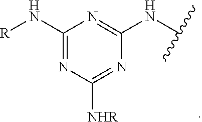

In some embodiments, antibody binding moiety is or comprises optionally substituted

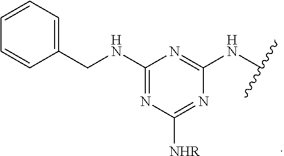

In some embodiments, antibody binding moiety is or comprises

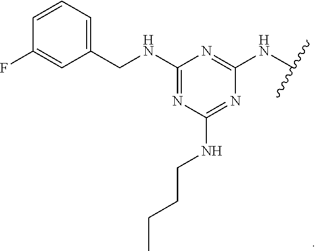 [SMILES: *Nc1nc(NCCCC)nc(NCc2cccc(F)c2)n1]

In some embodiments, an antibody binding moiety is or comprises optionally substituted

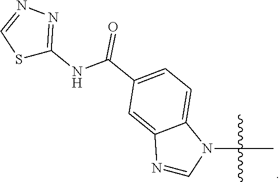 [SMILES: *n1cnc2cc(C(=O)Nc3nncs3)ccc21]

In some embodiments, an antibody binding moiety is or comprises

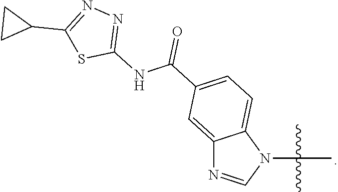 [SMILES: *n1cnc2cc(C(=O)Nc3nnc(C4CC4)s3)ccc21]

In some embodiments, an antibody binding moiety is or comprises

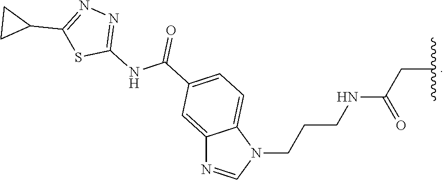 [SMILES: *CC(=O)NCCCn1cnc2cc(C(=O)Nc3nnc(C4CC4)s3)ccc21]

In some embodiments, an antibody binding moiety is or comprises optionally substituted

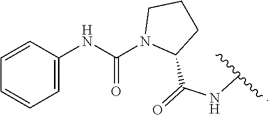 [SMILES: *NC(=O)[C@H]1CCCN1C(=O)Nc1ccccc1]

In some embodiments, an antibody binding moiety is or comprises

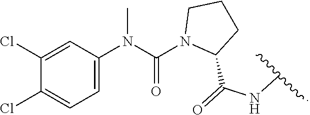 [SMILES: *NC(=O)[C@H]1CCCN1C(=O)N(C)c1ccc(Cl)c(Cl)c1]

In some embodiments, an antibody binding moiety is or comprises optionally substituted

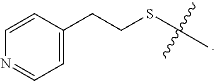 [SMILES: *SCCc1ccncc1]

In some embodiments, an antibody binding moiety is or comprises

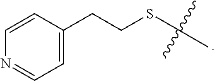 [SMILES: *SCCc1ccncc1]

In some embodiments, such antibody binding moieties are antibody binding moieties.

In some embodiments, antibody binding moiety is or comprises

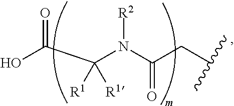

wherein each variable is independently as described herein. In some embodiments, m is 4 to 13. In some embodiments, an antibody binding moiety is or comprises

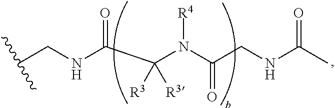

wherein b is 1-20, and each other variable is independently as described herein. In some embodiments, b is 4-13. In some embodiments, an antibody binding moiety, e.g., R^{c}-(Xaa)z-, is or comprises

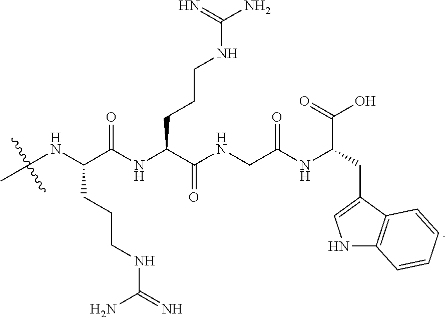 [SMILES: *N[C@@H](CCCNC(=N)N)C(=O)N[C@@H](CCCNC(=N)N)C(=O)NCC(=O)N[C@@H](Cc1c[nH]c2ccccc12)C(=O)O]

In some embodiments, an antibody binding moiety, e.g., R^{c}-(Xaa)z-, is or comprises

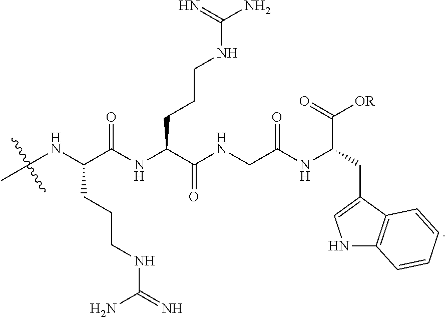

In some embodiments, an antibody binding moiety, e.g., R^{c}-(Xaa)z-, is or comprises

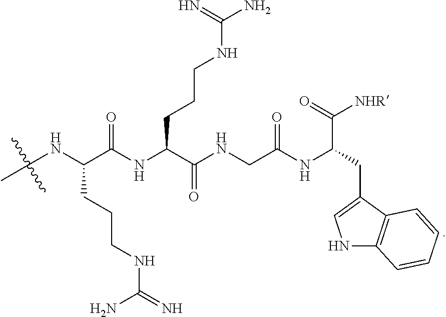

In some embodiments, an antibody binding moiety, e.g., R^{c}-(Xaa)z-, is or comprises

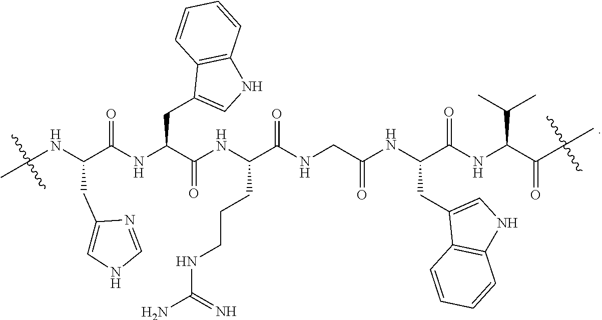 [SMILES: *N[C@@H](Cc1c[nH]cn1)C(=O)N[C@@H](Cc1c[nH]c2ccccc12)C(=O)N[C@@H](CCCNC(=N)N)C(=O)NCC(=O)N[C@@H](Cc1c[nH]c2ccccc12)C(=O)N[C@H](C(*)=O)C(C)C]

In some embodiments, an antibody binding moiety, e.g., R^{c}-(Xaa)z-, is or comprises

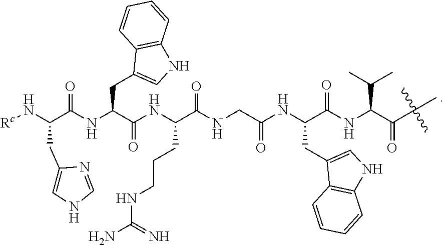

In some embodiments, an antibody binding moiety, e.g., R^{c}-(Xaa)z-, is or comprises

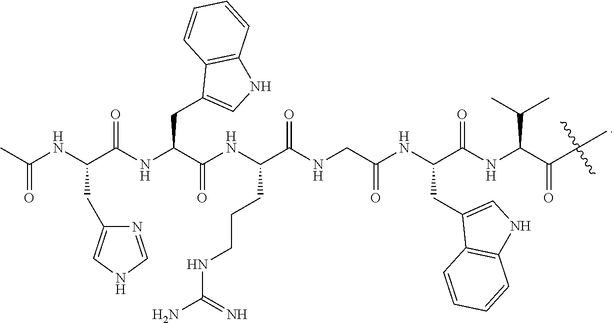 [SMILES: *C(=O)[C@@H](NC(=O)[C@H](Cc1c[nH]c2ccccc12)NC(=O)CNC(=O)[C@H](CCCNC(=N)N)NC(=O)[C@H](Cc1c[nH]c2ccccc12)NC(=O)[C@H](Cc1c[nH]cn1)NC(C)=O)C(C)C]

In some embodiments, an antibody binding moiety, e.g., R^{c}-(Xaa)z-, is or comprises

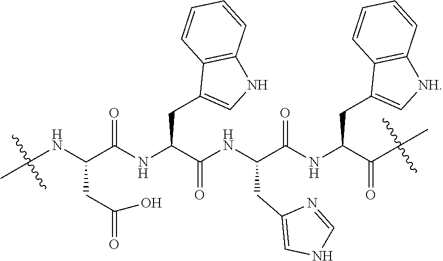 [SMILES: *N[C@@H](CC(=O)O)C(=O)N[C@@H](Cc1c[nH]c2ccccc12)C(=O)N[C@@H](Cc1c[nH]cn1)C(=O)N[C@@H](Cc1c[nH]c2ccccc12)C(*)=O]

In some embodiments, an antibody binding moiety, e.g., R^{c}-(Xaa)z-, is or comprises

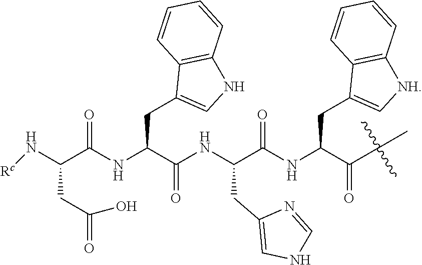

In some embodiments, an antibody binding moiety, e.g., R^{c}-(Xaa)z-, is or comprises

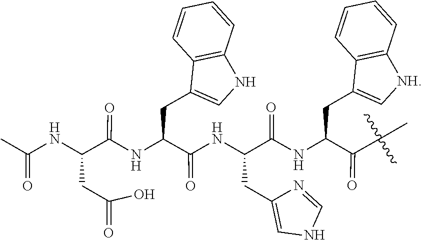 [SMILES: *C(=O)[C@H](Cc1c[nH]c2ccccc12)NC(=O)[C@H](Cc1c[nH]cn1)NC(=O)[C@H](Cc1c[nH]c2ccccc12)NC(=O)[C@H](CC(=O)O)NC(C)=O]

In some embodiments, an antibody binding moiety, e.g., R^{c}-(Xaa)z-, is or comprises

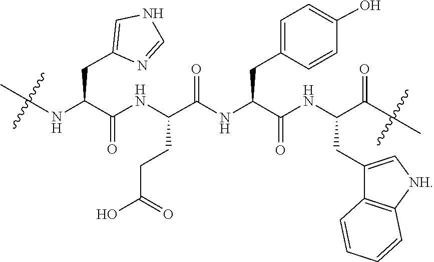 [SMILES: *N[C@@H](Cc1c[nH]cn1)C(=O)N[C@@H](CCC(=O)O)C(=O)N[C@@H](Cc1ccc(O)cc1)C(=O)N[C@@H](Cc1c[nH]c2ccccc12)C(*)=O]

In some embodiments, an antibody binding moiety, e.g., R^{c}-(Xaa)z-, is or comprises

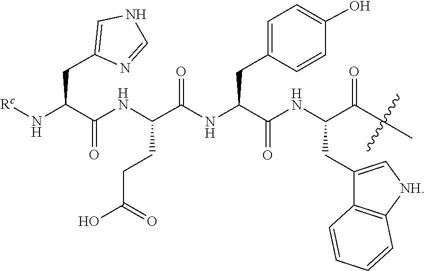

In some embodiments, an antibody binding moiety, e.g., R^{c}-(Xaa)z-, is or comprises

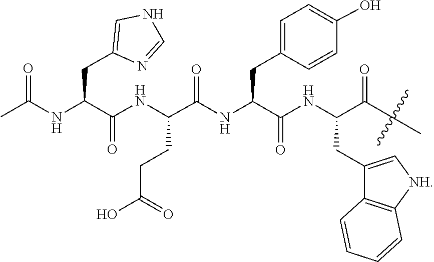 [SMILES: *C(=O)[C@H](Cc1c[nH]c2ccccc12)NC(=O)[C@H](Cc1ccc(O)cc1)NC(=O)[C@H](CCC(=O)O)NC(=O)[C@H](Cc1c[nH]cn1)NC(C)=O]

In some embodiments, an antibody binding moiety, e.g., R^{c}-(Xaa)z-, is or comprises

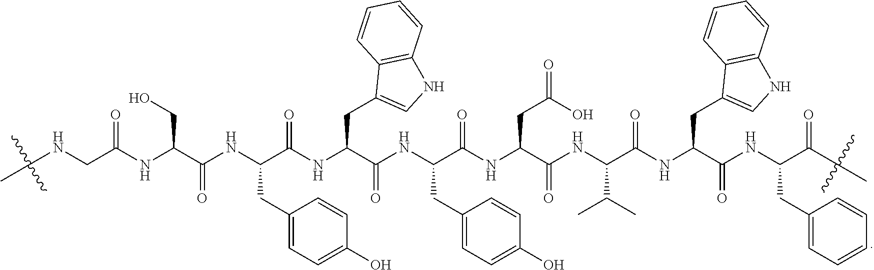 [SMILES: *NCC(=O)N[C@@H](CO)C(=O)N[C@@H](Cc1ccc(O)cc1)C(=O)N[C@@H](Cc1c[nH]c2ccccc12)C(=O)N[C@@H](Cc1ccc(O)cc1)C(=O)N[C@@H](CC(=O)O)C(=O)N[C@H](C(=O)N[C@@H](Cc1c[nH]c2ccccc12)C(=O)N[C@@H](Cc1ccccc1)C(*)=O)C(C)C]

In some embodiments, an antibody binding moiety, e.g., R^{c}-(Xaa)z-, is or comprises

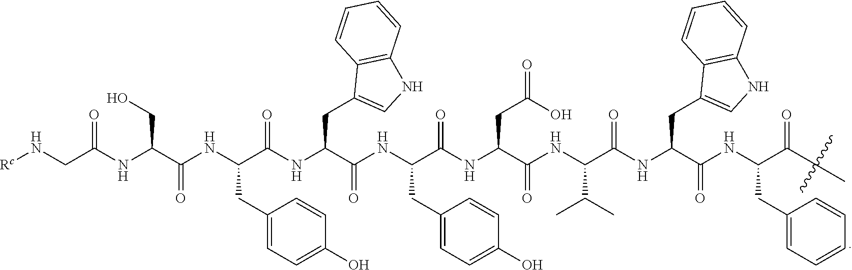

In some embodiments, an antibody binding moiety, e.g., R^{c}-(Xaa)z-, is or comprises

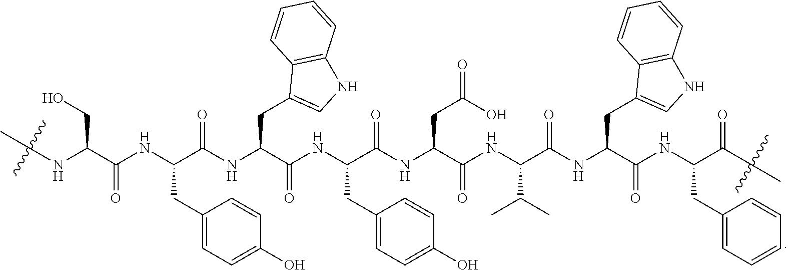 [SMILES: *N[C@@H](CO)C(=O)N[C@@H](Cc1ccc(O)cc1)C(=O)N[C@@H](Cc1c[nH]c2ccccc12)C(=O)N[C@@H](Cc1ccc(O)cc1)C(=O)N[C@@H](CC(=O)O)C(=O)N[C@H](C(=O)N[C@@H](Cc1c[nH]c2ccccc12)C(=O)N[C@@H](Cc1ccccc1)C(*)=O)C(C)C]

In some embodiments, an antibody binding moiety, e.g., R^{c}-(Xaa)z-, is or comprises

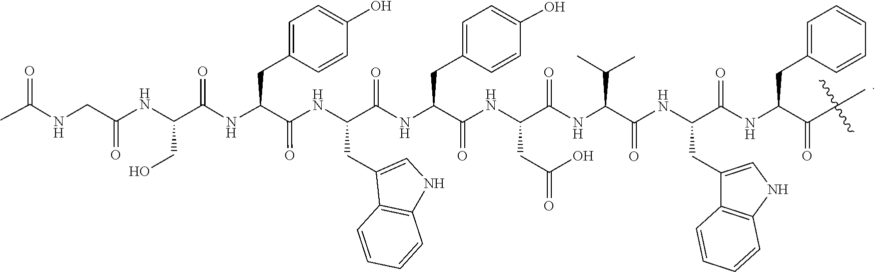 [SMILES: *C(=O)[C@H](Cc1ccccc1)NC(=O)[C@H](Cc1c[nH]c2ccccc12)NC(=O)[C@@H](NC(=O)[C@H](CC(=O)O)NC(=O)[C@H](Cc1ccc(O)cc1)NC(=O)[C@H](Cc1c[nH]c2ccccc12)NC(=O)[C@H](Cc1ccc(O)cc1)NC(=O)[C@H](CO)NC(=O)CNC(C)=O)C(C)C]

In some embodiments, an antibody binding moiety, e.g., R^{c}-(Xaa)z-, is or comprises

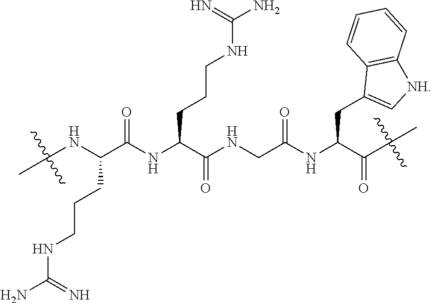 [SMILES: *N[C@@H](CCCNC(=N)N)C(=O)N[C@@H](CCCNC(=N)N)C(=O)NCC(=O)N[C@@H](Cc1c[nH]c2ccccc12)C(*)=O]

In some embodiments, an antibody binding moiety, e.g., R^{c}-(Xaa)z-, is or comprises

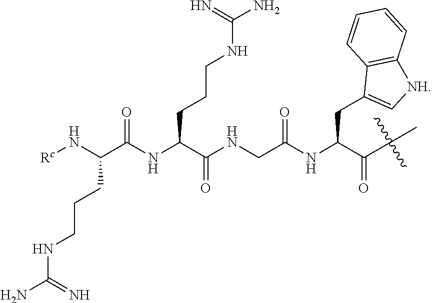

In some embodiments, an antibody binding moiety, e.g., R^{c}-(Xaa)z-, is or comprises

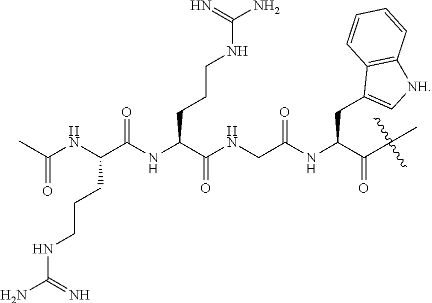 [SMILES: *C(=O)[C@H](Cc1c[nH]c2ccccc12)NC(=O)CNC(=O)[C@H](CCCNC(=N)N)NC(=O)[C@H](CCCNC(=N)N)NC(C)=O]

In some embodiments, —NH— is bonded to a R^{c} group. In some embodiments, R^{c} is R—C(O)—. In some embodiments, R^{c} is CH₃C(O)—. In some embodiments, such antibody binding moieties are antibody binding moieties.

In some embodiments, an antibody binding moiety, e.g.,

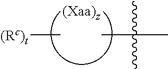

or R^{c}-(Xaa)z-, is or comprises

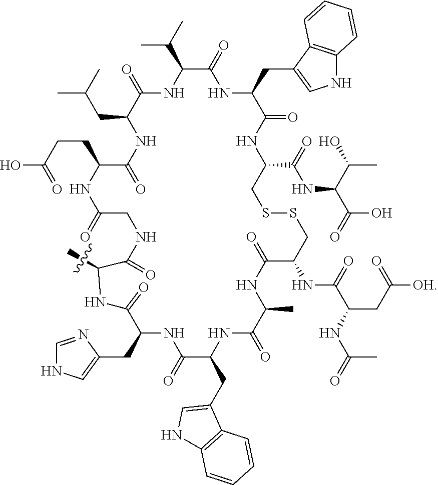

In some embodiments, an antibody binding moiety, e.g.,

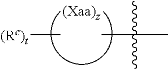

or R^{c}-(Xaa)z-, is or comprises

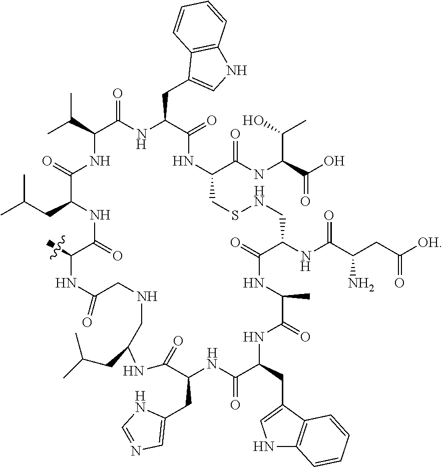

In some embodiments, an antibody binding moiety, e.g.,

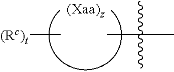

or R^{c}-(Xaa)z-, is or comprises

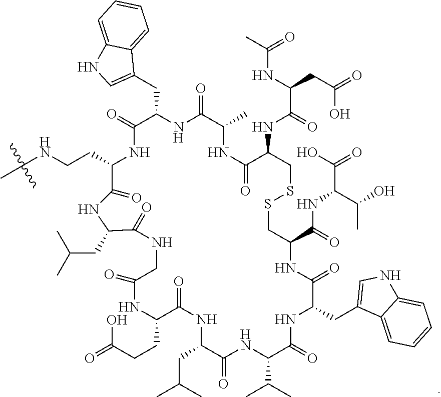

In some embodiments, an antibody binding moiety, e.g.,

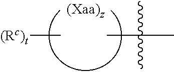

or R^{c}-(Xaa)z-, is or comprises

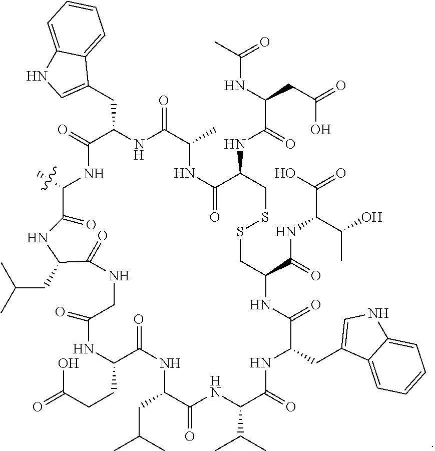

In some embodiments, an antibody binding moiety, e.g.,

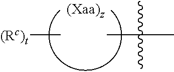

or R^{c}-(Xaa)z-, is or comprises

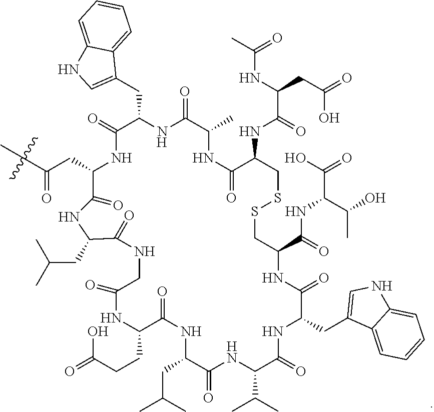

In some embodiments, an antibody binding moiety, e.g.,

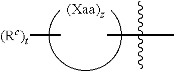

or R^{c}-(Xaa)z-, is or comprises

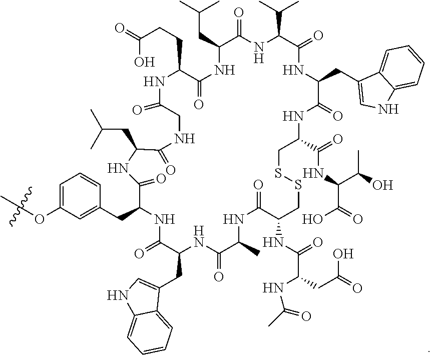 [SMILES: *Oc1cccc(C[C@@H]2NC(=O)[C@H](Cc3c[nH]c4ccccc34)NC(=O)[C@H](C)NC(=O)[C@@H](NC(=O)[C@H](CC(=O)O)NC(C)=O)CSSC[C@@H](C(=O)N[C@H](C(=O)O)[C@@H](C)O)NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC(=O)[C@H](CC(C)C)NC2=O)c1]

In some embodiments, an antibody binding moiety, e.g.,

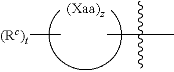

or R^{c}-(Xaa)z-, is or comprises

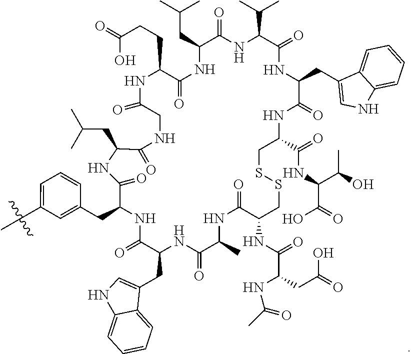 [SMILES: *c1cccc(C[C@@H]2NC(=O)[C@H](Cc3c[nH]c4ccccc34)NC(=O)[C@H](C)NC(=O)[C@@H](NC(=O)[C@H](CC(=O)O)NC(C)=O)CSSC[C@@H](C(=O)N[C@H](C(=O)O)[C@@H](C)O)NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC(=O)[C@H](CC(C)C)NC2=O)c1]

In some embodiments, an antibody binding moiety, e.g.,

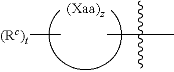

or R^{c}-(Xaa)z-, is or comprises

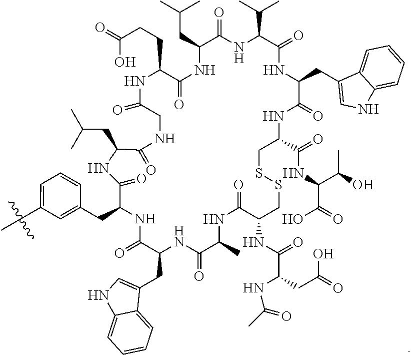 [SMILES: *c1cccc(C[C@@H]2NC(=O)[C@H](Cc3c[nH]c4ccccc34)NC(=O)[C@H](C)NC(=O)[C@@H](NC(=O)[C@H](CC(=O)O)NC(C)=O)CSSC[C@@H](C(=O)N[C@H](C(=O)O)[C@@H](C)O)NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC(=O)[C@H](CC(C)C)NC2=O)c1]

In some embodiments, an antibody binding moiety, e.g.,

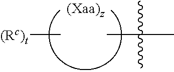

or R^{c}-(Xaa)z-, is or comprises

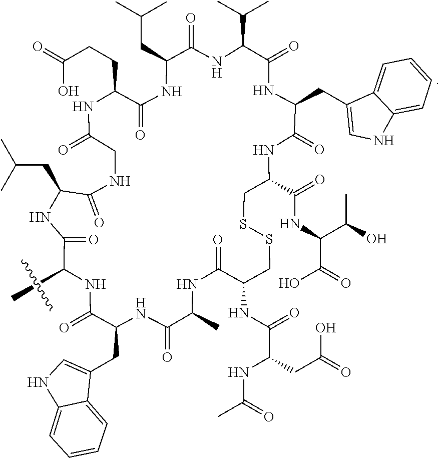

In some embodiments, such antibody binding moieties are antibody binding moieties.

In some embodiments, an antibody binding moiety, e.g., R^{c}-(Xaa)z-, is or comprises a Z33 peptide moiety. In some embodiments, an antibody binding moiety, e.g., R^{c}-(Xaa)z-, is or comprises -FNMQQQRRFYEALHDPNLNEEQRNAKIKSIRDD-NH₂ (SEQ ID NO:50) or a fragment thereof. In some embodiments, an antibody binding moiety, e.g., R^{c}-(Xaa)z-, is or comprises FNMQCQRRFYEALHDPNLNEEQRNAKIKSIRDDC (SEQ ID NO:51) or a fragment thereof. In some embodiments, an antibody binding moiety, e.g.,

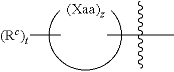

or R^{c}-(Xaa)z-, is or comprises a moiety of a peptide such as FNMQCQRRFYEALHDPNLNEEQRNAKIKSIRDDC (SEQ ID NO:51), RGNCAYHRGQLVWCTYH (SEQ ID NO:52), RGNCAYHKGQLVWCTYH (SEQ ID NO:53), RGNCKYHRGQLVWCTYH (SEQ ID NO:54), RGNCAWHRGKLVWCTYH (SEQ ID NO:55), RGNCKWHRGELVWCTYH (SEQ ID NO:56), RGNCKWHRGQLVWCTYH (SEQ ID NO:57), RGNCKYHLGELVWCTYH (SEQ ID NO:58), RGNCKYHLGQLVWCTYH (SEQ ID NO:59), DCKWHLGELVWCT (SEQ ID NO:60), DCKYHLGELVWCT (SEQ ID NO: 61), DCKWHRGELVWCT (SEQ ID NO:62), DCKWHLGQLVWCT (SEQ ID NO:63), DCKYHRGELVWCT (SEQ ID NO:64), DCKYHLGQLVWCT (SEQ ID NO:65), DCKWHRGQLVWCT (SEQ ID NO:66), DCKYHRGQLVWCT (SEQ ID NO:67), FNKQCQRRFYEALHDPNLNEEQRNARIRSIRDDC (SEQ ID NO:68), FNMQCQRRFYEALHDPNLNEEQRNARIRSIKDDC (SEQ ID NO:69), FNMQCQRRFYEALHDPNLNKEQRNARIRSIRDDC (SEQ ID NO:70), FNMQCQRRFYEALHDPNLNEEQRNARIRSIRDDC (SEQ ID NO:71), RGNCAWHLGQLVWCKYH (SEQ ID NO:72), RGNCAWHLGELVWCKYH (SEQ ID NO:73), RGNCAYHLGQLVWCTKH (SEQ ID NO:74), RGNCAYHLGQLVWCTYK (SEQ ID NO:75), RGNCAYHRGQLVWCTKH (SEQ ID NO:76), KNMQCQRRFYEALHDPNLNEEQRNARIRSIRDDC (SEQ ID NO:77), FNMQCQKRFYEALHDPNLNEEQRNARIRSIRDDC (SEQ ID NO:78), FNMQCQRRFYEAKHDPNLNEEQRNARIRSIRDDC (SEQ ID NO:79), FNMQCQRRFYEALHDPNLNEEQRKARIRSIRDDC (SEQ ID NO:80), FNMQCQRRFYEALHDPNLNKEQRNARIRSIRDDC (SEQ ID NO:70), FNMQCQRRFYEALHDPNLNEEQRNARIRSIKDDC (SEQ ID NO:69), FNKQCQRRFYEALHDPNLNEEQRNARIRSIRDDC (SEQ ID NO:68), FNMQCKRRFYEALHDPNLNEEQRNARIRSIRDDC SEQ ID NO:81), FNMQCQRRFYEALHDPNLNEEQRNARIRSIRKDC (SEQ ID NO:82), Fc-III, FcBP-2, Fc-III-4C,

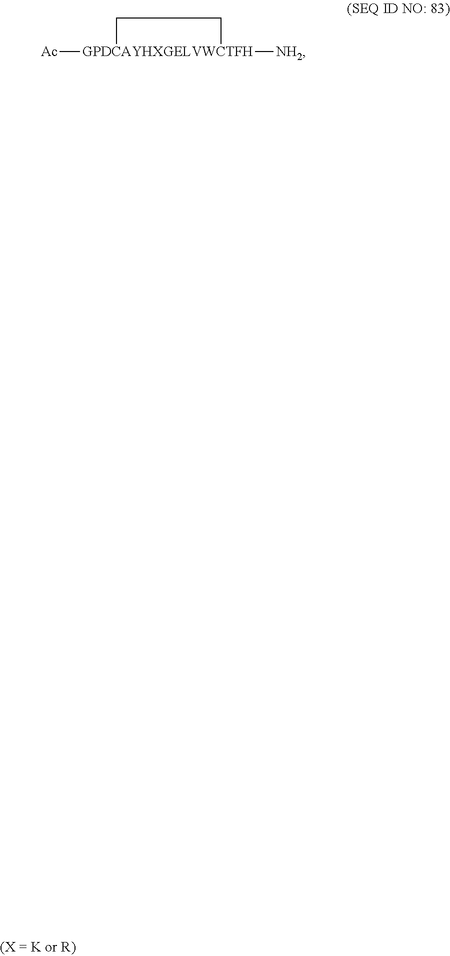

etc., wherein two cysteine residues may optionally form a disulfide bond. In some embodiments, in a peptide described herein, two cysteine residues form a disulfide bond. In some embodiments, a peptide, such as Z33, FNMQCQRRFYEALHDPNLNEEQRNAKIKSIRDDC (SEQ ID NO:51), RGNCAYHRGQLVWCTYH (SEQ ID NO:52), RGNCKYHRGQLVWCTYH (SEQ ID NO:54), RGNCAYHKGQLVWCTYH (SEQ ID NO:53), RGNCAWHRGKLVWCTYH (SEQ ID NO:55), RGNCKWHRGQLVWCTYH (SEQ ID NO:57), RGNCKWHRGELVWCTYH (SEQ ID NO:56), RGNCKYHLGELVWCTYH (SEQ ID NO:58), RGNCKYHLGQLVWCTYH (SEQ ID NO:59), DCKWHLGELVWCT (SEQ ID NO:60), DCKYHLGELVWCT (SEQ ID NO:61), DCKWHRGELVWCT (SEQ ID NO:62), DCKWHLGQLVWCT (SEQ ID NO:63), DCKYHRGELVWCT (SEQ ID NO:64), DCKYHLGQLVWCT (SEQ ID NO:65), DCKWHRGQLVWCT (SEQ ID NO:66), DCKYHRGQLVWCT (SEQ ID NO:67), FNKQCQRRFYEALHDPNLNEEQRNARIRSIRDDC (SEQ ID NO:68), FNMQCQRRFYEALHDPNLNEEQRNARIRSIKDDC (SEQ ID NO:69), FNMQCQRRFYEALHDPNLNKEQRNARIRSIRDDC SEQ ID NO:70), FNMQCQRRFYEALHDPNLNEEQRNARIRSIRDDC (SEQ ID NO:71), RGNCAWHLGQLVWCKYH (SEQ ID NO:72), RGNCAWHLGELVWCKYH (SEQ ID NO:73), RGNCAYHLGQLVWCTKH (SEQ ID NO:74), RGNCAYHLGQLVWCTYK (SEQ ID NO:75), RGNCAYHRGQLVWCTKH (SEQ ID NO:76), KNMQCQRRFYEALHDPNLNEEQRNARIRSIRDDC (SEQ ID NO:77), FNMQCQKRFYEALHDPNLNEEQRNARIRSIRDDC (SEQ ID NO:78), FNMQCQRRFYEAKHDPNLNEEQRNARIRSIRDDC (SEQ ID NO:79), FNMQCQRRFYEALHDPNLNEEQRKARIRSIRDDC (SEQ ID NO:80), FNMQCQRRFYEALHDPNLNKEQRNARIRSIRDDC (SEQ ID NO:70), FNMQCQRRFYEALHDPNLNEEQRNARIRSIKDDC (SEQ ID NO:69), FNKQCQRRFYEALHDPNLNEEQRNARIRSIRDDC (SEQ ID NO:68), FNMQCKRRFYEALHDPNLNEEQRNARIRSIRDDC (SEQ ID NO:81), FNMQCQRRFYEALHDPNLNEEQRNARIRSIRKDC (SEQ ID NO:82), Fc-III, FcBP-2, Fc-III-4C,

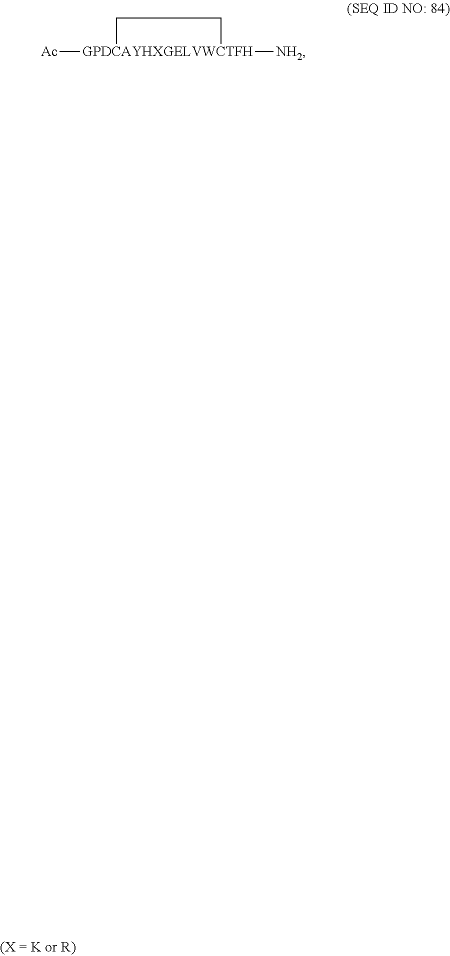

etc., is connected through its N-terminus, C-terminus, or a side chain (e.g., of K (e.g., of underlined K residues in RGNCAYHKGQLVWCTYH (SEQ ID NO:53), RGNCKYHRGQLVWCTYH (SEQ ID NO:54), RGNCAWHRGKLVWCTYH (SEQ ID NO:55), RGNCKWHRGELVWCTYH (SEQ ID NO:56), RGNCKWHRGQLVWCTYH (SEQ ID NO:57), RGNCKYHLGELVWCTYH (SEQ ID NO:58), RGNCKYHLGQLVWCTYH (SEQ ID NO:59), DCKWHLGELVWCT (SEQ ID NO:60), DCKYHLGELVWCT (SEQ ID NO:61), DCKWHRGELVWCT (SEQ ID NO:62), DCKWHLGQLVWCT (SEQ ID NO:63), DCKYHRGELVWCT (SEQ ID NO:64), DCKYHLGQLVWCT (SEQ ID NO:65), DCKWHRGQLVWCT (SEQ ID NO:66), DCKYHRGQLVWCT (SEQ ID NO:67), RGNCAWHLGQLVWCKYH (SEQ ID NO:72), FNKQCQRRFYEALHDPNLNEEQRNARIRSIRDDC (SEQ ID NO:68), FNMQCQRRFYEALHDPNLNEEQRNARIRSIKDDC (SEQ ID NO:69), FNMQCQRRFYEALHDPNLNKEQRNARIRSIRDDC (SEQ ID NO:70), RGNCAWHLGELVWCKYH (SEQ ID NO:73), RGNCAYHLGQLVWCTKH (SEQ ID NO:74), RGNCAYHLGQLVWCTYK (SEQ ID NO:75), RGNCAYHRGQLVWCTKH (SEQ ID NO:76), KNMQCQRRFYEALHDPNLNEEQRNARIRSIRDDC (SEQ ID NO:77), FNMQCQKRFYEALHDPNLNEEQRNARIRSIRDDC (SEQ ID NO:78), FNMQCQRRFYEAKHDPNLNEEQRNARIRSIRDDC (SEQ ID NO:79), FNMQCQRRFYEALHDPNLNEEQRKARIRSIRDDC (SEQ ID NO:80), FNMQCQRRFYEALHDPNLNKEQRNARIRSIRDDC (SEQ ID NO:70), FNMQCQRRFYEALHDPNLNEEQRNARIRSIKDDC (SEQ ID NO:69), FNKQCQRRFYEALHDPNLNEEQRNARIRSIRDDC (SEQ ID NO:68), FNMQCKRRFYEALHDPNLNEEQRNARIRSIRDDC (SEQ ID NO:81), FNMQCQRRFYEALHDPNLNEEQRNARIRSIRKDC (SEQ ID NO:82), etc.)). In some embodiments, one or more amino acid residues of a sequence may be independently and optionally replaced (e.g., 1-5), deleted (e.g., 1-5) and/or inserted (e.g., 1-5) as described herein. In some embodiments, an antibody binding moiety, e.g.,

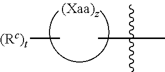

or R^{c}-(Xaa)z-, is or comprises -CXYHXXXLVWC-, -XCXYHXXXLVWC-, -CXYHXXXLVWCX-, —X₀₋₃CXYHXXXLVWCX₀₋₃-, -XCXYHXXXLVWCXXX--XXXCXYHXXXLVWCXXX-, wherein each X is independently an amino acid residue, and the two C residues optionally form a disulfide bond. In some embodiments, X⁸ (the X after H) is Orn. In some embodiments, X⁸ is Dab. In some embodiments, X⁸ is Lys(Ac). In some embodiments, X⁸ is Om(Ac). In some embodiments, X⁸ is Dab(Ac). In some embodiments, X⁸ is Arg. In some embodiments, X⁸ is Nle. In some embodiments, X⁸ is Nva. In some embodiments, X⁸ is Val. In some embodiments, X⁸ is Tle. In some embodiments, X⁸ is Leu. In some embodiments, X⁸ is Ala(tBu). In some embodiments, X⁸ is Cha. In some embodiments, X⁸ is Phe. In some embodiments, an antibody binding moiety, e.g.,

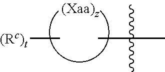

or R^{c}-(Xaa)z-, is or comprises DCAWHLGELVWCT (SEQ ID NO:35). In some embodiments, a C-terminus and/or a N-terminus of a protein agent/peptide agent moiety are independently capped (e.g., RC(O)— such as CH₃C(O)— for N-terminus, —N(R′)₂ such as —NH₂ for C-terminus, etc.). In some embodiments, such antibody binding moieties are antibody binding moieties. In some embodiments, as described herein, a residue may be modified or replaced for connection with another moiety, e.g., in some embodiments, H may be replaced with an amino acid residue comprises a side chain that contain —COOH or a salt or activated form thereof (e.g., D).

In some embodiments, an antibody binding moiety, e.g.,

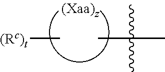

or R^{c}-(Xaa)z-, is or comprises (X₁₋₃)—C—(X₂)—H-(Xaa1)-G-(Xaa2)-L-V—W—C—(X₁₋₃), wherein each of X and Xaa is independently an amino acid residue and optionally not a cysteine residue. In some embodiments, Xaa1 is R, L, L, D, E, a 2-amino suberic acid residue, or a diaminopropionic acid residue. In some embodiments, Xaa2 is L, D, E, N, or Q. In some embodiments, Xaa1 is a lysine residue, a cysteine residue, an aspartic acid residue, a glutamic acid residue, a 2-amino suberic acid residue, or a diaminopropionic acid residue. In some embodiments, Xaa2 is a glutamic acid residue or an aspartic acid residue. In some embodiments, Xaa1 is an arginine residue or a leucine residue. In some embodiments, Xaa2 is a lysine residue, a glutamine residue, or an aspartic acid residue. In some embodiments, such antibody binding moieties are antibody binding moieties.

In some embodiments, an antibody binding moiety, e.g.,

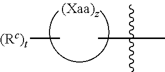

or R^{c}-(Xaa)z-, is or comprises (X1-3)-C-(Xaa3)-(xaa4)-H-(Xaa1)-G-(Xaa2)-L-V—W—C-(Xaa5)-(Xaa6)-(Xaa7), wherein each of X and Xaa is independently an amino acid residue and optionally not a cysteine residue. In some embodiments, Xaa3 is an alanine residue or a lysine residue. In some embodiments, Xaa4 is a tryptophan residue or a tyrosine residue. In some embodiments, Xaa1 is an arginine residue, a leucine residue, a lysine residue, an aspartic acid residue, a glutamic acid residue, a 2-amino suberic acid residue, or a diaminopropionic acid residue. In some embodiments, Xaa2 is a lysine residue, a glutamine residue, a glutamic acid residue, an asparagine residue, or an aspartic acid residue. In some embodiments, Xaa5 is a threonine residue or a lysine residue. In some embodiments, Xaa6 is a tyrosine residue, a lysine residue, or absent. In some embodiments, Xaa7 is a histidine residue, a lysine residue, or absent. In some embodiments, such antibody binding moieties are antibody binding moieties.

In some embodiments, an antibody binding moiety, e.g.,

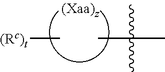

or R^{c}-(Xaa)z-, is or comprises D-C-(Xaa3)-(Xaa4)-H-(Xaa1)-G-(Xaa2)-L-V—W—C-(Xaa5)-(Xaa6)-(Xaa7), wherein each of X and Xaa is independently an amino acid residue and optionally not a cysteine residue. In some embodiments, Xaa3 is an alanine residue or a lysine residue. In some embodiments, Xaa4 is a tryptophan residue or a tyrosine residue. In some embodiments, Xaa1 is an arginine residue, a leucine residue, a lysine residue, an aspartic acid residue, a glutamic acid residue, a 2-amino suberic acid residue, or a diaminopropionic acid residue. In some embodiments, Xaa2 is a lysine residue, a glutamine residue, a glutamic acid residue, an asparagine residue, or an aspartic acid residue. In some embodiments, Xaa5 is a threonine residue or a lysine residue. In some embodiments, Xaa6 is a tyrosine residue, a lysine residue, or absent. In some embodiments, Xaa7 is a histidine residue, a lysine residue, or absent. In some embodiments, such antibody binding moieties are antibody binding moieties.

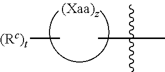

In some embodiments, an antibody binding moiety, e.g., or R^{c}-(Xaa)z-, is or comprises D-C-(Xaa3)-(Xaa4)-H-(Xaa1)-G-(Xaa2)-L-V—W—C-T, wherein each of X and Xaa is independently an amino acid residue and optionally not a cysteine residue. In some embodiments, Xaa3 is an alanine residue or a lysine residue. In some embodiments, Xaa4 is a tryptophan residue or a tyrosine residue. In some embodiments, Xaa1 is an arginine residue, a leucine residue, a lysine residue, an aspartic acid residue, a glutamic acid residue, a 2-amino suberic acid residue, or a diaminopropionic acid residue. In some embodiments, Xaa2 is a lysine residue, a glutamine residue, a glutamic acid residue, an asparagine residue, or an aspartic acid residue. In some embodiments, such antibody binding moieties are antibody binding moieties.

In some embodiments, an antibody binding moiety, e.g.,

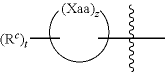

or R^{c}-(Xaa)z-, is or comprises R-G-N—C-(Xaa3)-(Xaa4)-H-(Xaa1)-G-(Xaa2)-L-V—W—C-(Xaa5)-(Xaa6)-(Xaa7), wherein each of X and Xaa is independently an amino acid residue and optionally not a cysteine residue. In some embodiments, Xaa3 is an alanine residue or a lysine residue. In some embodiments, Xaa4 is a tryptophan residue or a tyrosine residue. In some embodiments, Xaa1 is an arginine residue, a leucine residue, a lysine residue, an aspartic acid residue, a glutamic acid residue, a 2-amino suberic acid residue, or a diaminopropionic acid residue. In some embodiments, Xaa2 is a lysine residue, a glutamine residue, a glutamic acid residue, an asparagine residue, or an aspartic acid residue. In some embodiments, Xaa5 is a threonine residue or a lysine residue. In some embodiments, Xaa6 is a tyrosine residue, a lysine residue, or absent. In some embodiments, Xaa7 is a histidine residue, a lysine residue, or absent. In some embodiments, such antibody binding moieties are antibody binding moieties.

In some embodiments, antibody binding moieties, e.g., various antibody binding moieties described above, are protein binding moieties. In some embodiments, antibody binding moieties are antibody binding moieties. In some embodiments, LG is or comprises such an antibody binding moiety. In some embodiments, LG is or comprises a protein binding moiety. In some embodiments, LG is or comprises an antibody binding moiety.

In some embodiments, antibody binding moieties, e.g., antibody binding moieties, and useful technologies for developing and/or assessing such moieties are described in, e.g., Alves, Langmuir 2012, 28, 9640-9648; Choe et al., Materials 2016, 9, 994; doi:10.3390/ma9120994; Gupta et al., Nature Biomedical Engineering, vol. 3, 2019, 917-929; Muguruma, et al., ACS Omega 2019, 4, 14390-14397, doi: 10.1021/acsomega.9b01104; Yamada, et al., Angew Chem Int Ed Engl. 2019 Apr. 16; 58(17):5592-5597, doi: 10.1002/anie.201814215; Kruljec, et al., Bioconjug Chem. 2017, 28(8): 2009-2030, doi: 10.1021/acs.bioconjchem.7b00335 (e.g., Fabsorbent, triazines, etc.); Kruljec, et al., Bioconjugate Chem. 2018, 29, 8, 2763-2775, doi: 10.1021/acs.bioconjchem.8b00395; WO2012017021A2, etc., the binding moieties (e.g., antibody binding moieties) of each of which are incorporated herein by reference.

In some embodiments, an antibody binding moiety, e.g., a protein binding moiety (e.g., an antibody binding moiety), is an affinity substance described in AU 2018259856 or WO 2018199337, the affinity substance of each of which is incorporated herein by reference.

In some embodiments, an antibody binding moiety, e.g., an antibody binding moiety, is or comprises an adapter protein agent, e.g., as described in Hui, et al., Bioconjugate Chem. 2015, 26, 1456-1460, doi: 10.1021/acs.bioconjchem. 5b00275. In some embodiments, when utilized in accordance with the present disclosure, adapter proteins do not require reactive residues (e.g., BPA) to achieve one or more or all advantages.

In some embodiments, antibody binding moiety, e.g., an antibody binding moiety is or comprises a triazine moiety, e.g., one described in US 2009/0286693. In some embodiments, an antibody binding moiety, e.g., an antibody binding moiety is of such a structure that its corresponding compound is a compound described in US 2009/0286693, the compounds of which are independently incorporated herein by reference. In some embodiments, an antibody binding moiety, e.g., an antibody binding moiety, is ABT. In some embodiments, ABT is of such a structure that H-ABT is a compound described in US 2009/0286693, the compounds of which are independently incorporated herein by reference. In some embodiments, such a compound can bind to an antibody. In some embodiments, such a compound can bind to Fc region of an antibody.

In some embodiments, an antibody binding moiety, e.g., an antibody binding moiety is or comprises a triazine moiety, e.g., one described in Teng, et al., A strategy for the generation of biomimetic ligands for affinity chromatography. Combinatorial synthesis and biological evaluation of an IgG binding ligand, J. Mol. Recognit. 1999; 12:67-75 (“Teng”). In some embodiments, an antibody binding moiety, e.g., an antibody binding moiety is of such a structure that its corresponding compound is a compound described in Teng, the compounds of which are independently incorporated herein by reference. In some embodiments, an antibody binding moiety, e.g., an antibody binding moiety, ABT is of such a structure that H-ABT is a compound described in Teng, the compounds of which are independently incorporated herein by reference. In some embodiments, such a compound can bind to an antibody. In some embodiments, such a compound can bind to Fc region of an antibody.

In some embodiments, an antibody binding moiety, e.g., an antibody binding moiety is a triazine moiety, e.g., one described in Uttamchandani, et al., Microarrays of Tagged Combinatorial Triazine Libraries in the Discovery of Small-Molecule Ligands of Human IgG, J Comb Chem. 2004 November-December; 6(6):862-8 (“Uttamchandani”). In some embodiments, an antibody binding moiety, e.g., an antibody binding moiety is of such a structure that its corresponding compound is a compound described in Uttamchandani, the compounds of which are independently incorporated herein by reference. In some embodiments, an antibody binding moiety, e.g., an antibody binding moiety, ABT is of such a structure that H-ABT is a compound described in Uttamchandani, the compounds of which are independently incorporated herein by reference. In some embodiments, such a compound can bind to an antibody. In some embodiments, such a compound can bind to Fc region of an antibody.

In some embodiments, an antibody binding moiety binds to one or more binding sites of protein A. In some embodiments, an antibody binding moiety binds to one or more binding sites of protein G. In some embodiments, an antibody binding moiety binds to one or more binding sites of protein L. In some embodiments, an antibody binding moiety binds to one or more binding sites of protein Z. In some embodiments, an antibody binding moiety binds to one or more binding sites of protein LG. In some embodiments, an antibody binding moiety binds to one or more binding sites of protein LA. In some embodiments, an antibody binding moiety binds to one or more binding sites of protein AG. In some embodiments, an antibody binding moiety is described in Choe, W., Durgannavar, T. A., & Chung, S. J. (2016). Fc-binding ligands of immunoglobulin G: An overview of high affinity proteins and peptides. Materials, 9(12). https://doi.org/10.3390/ma9120994.

In some embodiments, an antibody binding moiety, e.g., an antibody binding moiety can bind to a nucleotide-binding site. In some embodiments, an antibody binding moiety, e.g., an antibody binding moiety is a small molecule moiety that can bind to a nucleotide-binding site. In some embodiments, a small molecule is tryptamine. In some embodiments, an antibody binding moiety, e.g., an antibody binding moiety, ABT is of such a structure that H-ABT is tryptamine. Certain useful technologies were described in Mustafaoglu, et al., Antibody Purification via Affinity Membrane Chromatography Method Utilizing Nucleotide Binding Site Targeting With A Small Molecule, Analyst. 2016 November 28; 141(24): 6571-6582.

Many technologies are available for identifying and/or assessing and/or characterizing antibody binding moieties, including protein binding moieties (e.g., antibody binding moieties such as universal antibody binding moieties), and/or their utilization in provided technologies, e.g., those described in WO/2019/023501, the technologies of which are incorporated herein by reference. In some embodiments, an antibody binding moiety is a moiety (e.g., small molecule moiety, peptide moiety, nucleic acid moiety, etc.) that can selectively bind to IgG, and when used in provided technologies can provide and/or stimulate ADCC and/or ADCP. In some embodiments, peptide display technologies (e.g., phase display, non-cellular display, etc.) can be utilized to identify antibody binding moieties. In some embodiments, an antibody binding moiety is a moiety (e.g., small molecule moiety, peptide moiety, nucleic acid moiety, etc.) that can bind to IgG and optionally can compete with known antibody binders, e.g., protein A, protein G, protein L, etc.

As appreciated by those skilled in the art, antibodies of various properties and activities (e.g., antibodies recognizing different antigens, having optional modifications, etc.) may be targeted by antibody binding moieties described in the present disclosure. In some embodiments, such antibodies include antibodies administered to a subject, e.g., for therapeutic purposes. In some embodiments, antibody binding moieties described herein may bind antibodies toward different antigens and are useful for conjugating moieties of interest with various antibodies.

In some embodiments, an antibody binding moiety, e.g., an antibody binding moiety, is or comprises a meditope agent moiety. In some embodiments, a meditope agent is described in, e.g., US 2019/0111149.

In some embodiments, an antibody binding moiety, e.g., an antibody binding moiety, can bind to human IgG. In some embodiments, an antibody binding moiety, e.g., an antibody binding moiety, can bind to rabbit IgG. In some embodiments, an antibody binding moiety, e.g., an antibody binding moiety, binds to IgG1. In some embodiments, an antibody binding moiety, e.g., an antibody binding moiety, binds to IgG2. In some embodiments, an antibody binding moiety, e.g., an antibody binding moiety, binds to IgG3. In some embodiments, an antibody binding moiety, e.g., an antibody binding moiety, binds to IgG4. In some embodiments, an antibody binding moiety, e.g., an antibody binding moiety, binds to IgG1, IgG2 and/or IgG4. In some embodiments, an antibody binding moiety, e.g., an antibody binding moiety, binds to IgG1, IgG2 and IgG4.

In some embodiments,

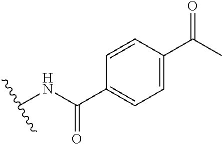 [SMILES: *NC(=O)c1ccc(C(C)=O)cc1]

is utilized in a reference technology as a non-antibody binding moiety. In some embodiments, CH₃— is utilized in a reference technology a non-antibody binding moiety. In some embodiments, CH₃C(O)— is utilized in a reference technology a non-antibody binding moiety. In some embodiments, CH₃C(O)NH— is utilized in a reference technology a non-antibody binding moiety. In some embodiments, CH₃C(O)NHCH₂— is utilized in a reference technology a non-antibody binding moiety. In some embodiments, CH₃CH₂— is utilized in a reference technology a non-antibody binding moiety. In some embodiments, CH₃CH₂NH— is utilized in a reference technology a non-antibody binding moiety. In some embodiments, CH₃CH₂NHC(O)— is utilized in a reference technology a non-antibody binding moiety.

In some embodiments, antibody binding moieties (e.g., antibody binding moieties) bind to targets (e.g., antibody agents for antibody binding moieties) with a Kd that is about 1 mM-1 pM or less. In some embodiments, a Kd is about 1 mM, 0.5 mM, 0.2 mM, 0.1 mM, 0.05 mM, 0.02 mM, 0.01 mM, 0.005 mM, 0.002 mM, 0.001 mM, 500 nM, 200 nM, 100 nM, 50 nM, 20 nM, 10 nM, 5 nM, 2 nM, 1 nM, 0.5 nM, 0.2 nM, 0.1 nM, or less. In some embodiments, Kd is about 1 mM or less. In some embodiments, Kd is about 0.5 mM or less. In some embodiments, Kd is about 0.1 mM or less. In some embodiments, Kd is about 0.05 mM or less. In some embodiments, Kd is about 0.01 mM or less. In some embodiments, Kd is about 0.005 mM or less. In some embodiments, Kd is about 0.001 mM or less. In some embodiments, Kd is about 500 nM or less. In some embodiments, Kd is about 200 nM or less. In some embodiments, Kd is about 100 nM or less. In some embodiments, Kd is about 50 nM or less. In some embodiments, Kd is about 20 nM or less. In some embodiments, Kd is about 10 nM or less. In some embodiments, Kd is about 5 nM or less. In some embodiments, Kd is about 2 nM or less. In some embodiments, Kd is about 1 nM or less. For example, in some embodiments, antibody binding moieties bind to IgG antibody agents with Kd described herein.

As appreciated by those skilled in the art, antibodies of various properties and activities (e.g., antibodies recognizing different antigens, having optional modifications, etc.) may be recruited by antibody binding moieties described in the present disclosure. In some embodiments, such antibodies include antibodies administered to a subject, e.g., for therapeutic purposes. In some embodiments, antibodies recruited by antibody binding moieties comprise antibodies toward different antigens. In some embodiments, antibodies recruited by antibody binding moieties comprise antibodies whose antigens are not present on the surface or cell membrane of target cells (e.g., target cells such as cells infected by SARS-CoV-2). In some embodiments, antibodies recruited by antibody binding moieties comprise antibodies which are not targeting antigens present on surface or cell membrane of targets (e.g., target cells such as cells infected by SARS-CoV-2). In some embodiments, antigens on surface of target cells may interfere with the structure, conformation, and/or one or more properties and/or activities of recruited antibodies which bind such antigens. In some embodiments, as appreciated by those skilled in the art, provided technologies comprise universal antibody binding moieties which recruit antibodies of diverse specificities, and no more than 1%, 2%, 5%, 10%, 15%, 20%, 25%, 30%, 35%, 40%, 45%, 50%, 55%, 60%, 65%, 70%, 75%, 80%, 85%, 90%, 95% or 99% percent of recruited antibodies are toward the same antigen, protein, lipid, carbohydrate, etc. Among other things, one advantage of the present disclosure is that provided technologies comprising universal antibody binding moieties can utilize diverse pools of antibodies such as those present in serum. In some embodiments, universal antibody binding moieties of the present disclosure (e.g., those in ARMs) are contacted with a plurality of antibodies, wherein no more than 1%, 2%, 5%, 10%, 15%, 20%, 25%, 30%, 35%, 40%, 45%, 50%, 55%, 60%, 65%, 70%, 75%, 80%, 85%, 90%, 95% or 99% percent of the plurality of antibodies are toward the same antigen, protein, lipid, carbohydrate, etc. In some embodiments, recruited antibodies are those in IVIG. In some embodiments, IVIG may be administered prior to, concurrently with or subsequently to an agent or composition. Among other things, antibodies of various types of immunoglobulin structures may be recruited. In some embodiments, one or more subclasses of IgG are recruited. In some embodiments, recruited antibodies comprise IgG1. In some embodiments, recruited antibodies comprise IgG2. In some embodiments, recruited antibodies comprise IgG3. In some embodiments, recruited antibodies comprise IgG4. In some embodiments, recruited antibodies are or comprise IgG1 and IgG2. In some embodiments, recruited antibodies are or comprise IgG1, IgG2 and IgG4. In some embodiments, recruited antibodies are or comprise IgG1, IgG2, IgG3 and IgG4. Recruited antibodies may interact various types of receptors, e.g., those expressed by various types of immune cells. In some embodiments, recruited antibodies can effectively interact various types of Fc receptors and provide desired immune activities. In some embodiments, recruited antibodies can recruit immune cells. In some embodiments, recruited antibodies can effectively interact with hFcγRIIIA. In some embodiments, recruited antibodies can effectively interact with hFcγRIIIA on macrophages. In some embodiments, macrophages are recruited to provide ADCC and/or ADCP activities toward a virus, e.g., a SARS-CoV-2 virus, and/or cells infected thereby. In some embodiments, NK cells are recruited to provide immune activities. In some embodiments, recruited antibodies can effectively interact with hFcγRIIA. In some embodiments, recruited antibodies can effectively interact with hFcγRIIA on dendritic cells. In some embodiments, antibody moieties in agents of the present disclosure comprise one or more properties, structures and/or activities of recruited antibodies described herein.

### SARS-CoV-2

It is reported that SARS-CoV-2 may belong to lineage B beta-coronavirus and can cause severe respiratory problems. Coughing, fever, difficulties in breathing and/or shortage of breath are reported to be among the common symptoms. Infection by SARS-CoV-2 is reported to lead to COVID-19. SARS-CoV-2 has caused a large number of confirmed cases and deaths globally.

Reportedly, SARS-CoV-2 can utilize human angiotensin-converting enzyme 2 (ACE2) as a receptor to infect human cells. There have been reports that SARS-CoV-2 spike (S) protein S2 subunit plays an important role in mediating virus fusion with and entry into the host cell, in which a heptad repeat 1 (HR1) and heptad repeat 2 (HR2) can interact to form six-helical bundle (6-HB), in some cases, reportedly bringing viral and cellular membranes in close proximity for fusion.

Genetic variations have been reported for SARS-CoV-2. In some embodiments, provided technologies can target one or more or all SARS-CoV-2 variants (e.g., by targeting specific or universal elements).

### Amino Acids

In some embodiments, provided compounds and agents may comprise one or more amino acid moieties, e.g., in antibody binding moieties, linker moieties, etc. Amino acid moieties can either be those of natural amino acids or unnatural amino acids. In some embodiments, an amino acid has the structure of formula A-I:

NH(R^{a1})-L^{a1}-C(R^{a2})(R^{a3})-L^{a2}-COOH, A-I

or a salt thereof, wherein:
each of R^{a1}, R^{a2} and R^{a3} is independently -L^{a}-R′ or an amino acid side chain;
each of L^{a1} and L^{a2} is independently L^{a};
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.
   In some embodiments, an amino acid residue, e.g., of an amino acid having the structure of formula A-I, has the structure of —N(R^{a1})-L^{a1}-C(R²)(R^{a3})-L^{a2}-CO—. In some embodiments, each amino acid residue in a peptide independently has the structure of —N(R^{a1})-L^{a1}-C(R^{a2})(R^{a3})-L^{a2}-CO—.

In some embodiments, the present disclosure provides a derivative of an amino acid of formula A-I or a salt thereof. In some embodiments, a derivative is an ester. In some embodiments, the present disclosure provides a compound of formula NH(R^{a1})-L^{a1}-C(R^{a2})(R^{a3})-L^{a2}-COOR^{CT} or salt thereof, wherein R^{CT} is R′ and each other variable is independently as described herein. In some embodiments, R^{CT} is R. In some embodiments, R^{CT} is optionally substituted aliphatic. In some embodiments, R^{CT} is t-butyl.

In some embodiments, L^{a1} is a covalent bond. In some embodiments, a compound of formula A-I is of the structure NH(R^{a1})—C(R^{a2})(R^{a3})-L^{a2}-COOH. In some embodiments, L^{a2} is —CH₂SCH₂—.

In some embodiments, L^{a2} is a covalent bond. In some embodiments, a compound of formula A-I is of the structure NH(R^{a1})-L^{a1}-C(R^{a2})(R^{a3})—COOH. In some embodiments, an amino acid residue has the structure of —N(R^{a1})-L^{a1}-C(R²)(R^{a3})—CO—. In some embodiments, L^{a1} is —CH₂CH₂S—. In some embodiments, L^{a1} is —CH₂CH₂S—, wherein the CH₂ is bonded to NH(R^{a1})

In some embodiments, L^{a1} is a covalent bond and L^{a2} is a covalent bond. In some embodiments, a compound of formula A-I is of the structure NH(R^{a1})—C(R^{a2})(R^{a3})—COOH. In some embodiments, a compound of formula A-I is of the structure NH(R^{a1})—CH(R^{a2})—COOH. In some embodiments, a compound of formula A-I is of the structure NH(R^{a1})—CH(R^{a3})—COOH. In some embodiments, a compound of formula A-I is of the structure NH₂—CH(R^{a2})—COOH. In some embodiments, a compound of formula A-I is of the structure NH₂—CH(R^{a3})—COOH. In some embodiments, an amino acid residue has the structure of —N(R^{a1})—C(R^{a2})(R^{a3})—CO—. In some embodiments, an amino acid residue has the structure of —N(R^{a1})—CH(R^{a2})—CO—. In some embodiments, an amino acid residue has the structure of —N(R^{a1})—CH(R^{a3})—CO—. In some embodiments, an amino acid residue has the structure of —NH—CH(R^{a2})—CO—. In some embodiments, an amino acid residue has the structure of —NH—CH(R^{a3})—CO—.

In some embodiments, L^{a} is a covalent bond. In some embodiments, L^{a} is optionally substituted C₁₋₆ bivalent aliphatic. In some embodiments, L^{a} is optionally substituted C₁₋₆ alkylene. In some embodiments, L^{a} is —CH₂—. In some embodiments, L^{a} is —CH₂CH₂—. In some embodiments, L^{a} is —CH₂CH₂CH₂—.

In some embodiments, L^{a} is bivalent optionally substituted C₁₋₂₀ aliphatic, wherein one or more methylene units are independently replaced with —C(O)—, —N(R′)—, -Cy-, and/or —O—. In some embodiments, L^{a} is bivalent optionally substituted C₁₋₂₀ aliphatic, wherein one or more methylene units are independently replaced with —C(O)N(R′)—, -Cy-, and —O—. In some embodiments, L^{a} is bivalent optionally substituted C₁₋₂₀ aliphatic, wherein two or more methylene units are independently replaced with —C(O)N(R′)—, and -Cy- in addition to other optional replacements. In some embodiments, -Cy- is optionally substituted. In some embodiments, -Cy- is optionally substituted with an electron-withdrawing group as described herein. In some embodiments, -Cy- is substituted with one or more —F. In some embodiments, -Cy- is optionally substituted 1,3-phenylene. In some embodiments, -Cy- is optionally substituted 1,4-phenylene. In some embodiments, L^{a} is or comprises

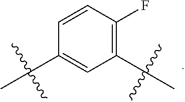 [SMILES: *c1ccc(F)c(*)c1]

In some embodiments, L^{a} is or comprises

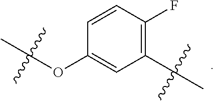 [SMILES: *Oc1ccc(F)c(*)c1]

In some embodiments, L^{a} is or comprises

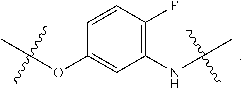 [SMILES: *Nc1cc(O*)ccc1F]

In some embodiments, L^{a} is or comprises

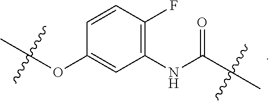 [SMILES: *Oc1ccc(F)c(NC(*)=O)c1]

In some embodiments, L^{a} is or comprises

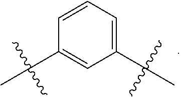 [SMILES: *c1cccc(*)c1]

In some embodiments, L^{a} is or comprises

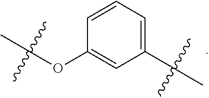 [SMILES: *Oc1cccc(*)c1]

In some embodiments, L^{a} is or comprises

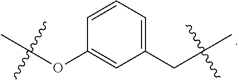 [SMILES: *Cc1cccc(O*)c1]

In some embodiments, L^{a} is or comprises

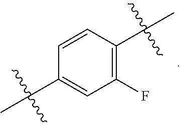 [SMILES: *c1ccc(*)c(F)c1]

In some embodiments, L^{a} is or comprises

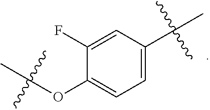 [SMILES: *Oc1ccc(*)cc1F]

In some embodiments, L^{a} is or comprises

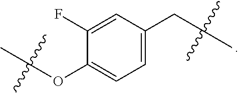 [SMILES: *Cc1ccc(O*)c(F)c1]

In some embodiments, L^{a} is or comprises

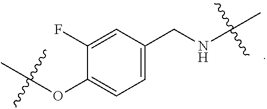 [SMILES: *NCc1ccc(O*)c(F)c1]

In some embodiments, L^{a} is or comprises

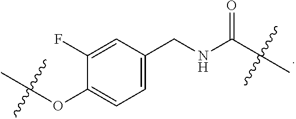 [SMILES: *Oc1ccc(CNC(*)=O)cc1F]

In some embodiments, L^{a} is or comprises

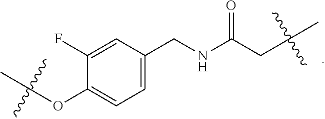 [SMILES: *CC(=O)NCc1ccc(O*)c(F)c1]

In some embodiments, L^{a} is or comprises

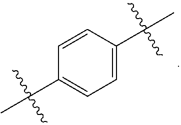 [SMILES: *c1ccc(*)cc1]

In some embodiments, L^{a} is or comprises

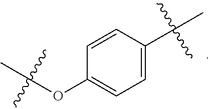 [SMILES: *Oc1ccc(*)cc1]

In some embodiments, L^{a} is or comprises

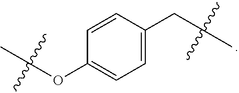 [SMILES: *Cc1ccc(O*)cc1]

In some embodiments, L^{a} is or comprises

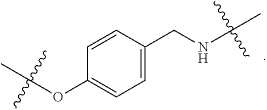 [SMILES: *NCc1ccc(O*)cc1]

In some embodiments, L^{a} is or comprises

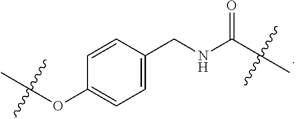 [SMILES: *Oc1ccc(CNC(*)=O)cc1]

In some embodiments, L^{a} is or comprises

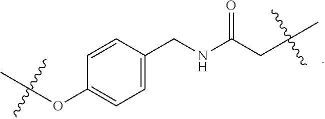 [SMILES: *CC(=O)NCc1ccc(O*)cc1]

In some embodiments, R′ is R. In some embodiments, R^{a1} is R, wherein R is as described in the present disclosure. In some embodiments, R^{a1} is R, wherein R methyl. In some embodiments, R^{a2} is R, wherein R is as described in the present disclosure. In some embodiments, R^{a3} is R, wherein R is as described in the present disclosure. In some embodiments, each of R^{a1}, R^{a2}, and R^{a3} is independently R, wherein R is as described in the present disclosure.

In some embodiments, R^{a1} is hydrogen. In some embodiments, R^{a1} is a protective group. In some embodiments, R^{a1} is -Fmoc. In some embodiments, R^{a1} is -Dde.

In some embodiments, each of R^{a1}, R^{a2} and R^{a3} is independently -L^{a}-R′.

In some embodiments, R^{a2} is hydrogen. In some embodiments, R^{a3} is hydrogen. In some embodiments, R^{a1} is hydrogen, and at least one of R^{a2} and R^{a3} is hydrogen. In some embodiments, R^{a1} is hydrogen, one of R^{a2} and R^{a3} is hydrogen, and the other is not hydrogen. In some embodiments, R^{a2} is -L^{a}-R and R^{a3} is —H. In some embodiments, R^{a3} is -L^{a}-R and R^{a2} is —H. In some embodiments, R^{a2} is —CH₂—R and R^{a3} is —H. In some embodiments, R^{a3} is —CH₂—R and R^{a2} is —H. In some embodiments, R^{a2} is R and R^{a3} is —H. In some embodiments, R^{a3} is R and R^{a2} is —H.

In some embodiments, R^{a2} is -L^{a}-R, wherein R is as described in the present disclosure. In some embodiments, R^{a2} is -L^{a}-R, wherein R is an optionally substituted group selected from C₃₋₃₀ cycloaliphatic, C₅₋₃₀ aryl, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, R^{a2} is -L^{a}-R, wherein R is an optionally substituted group selected from C₆₋₃₀ aryl and 5-30 membered heteroaryl having 1-10 heteroatoms independently selected from oxygen, nitrogen, sulfur, phosphorus and silicon. In some embodiments, R^{a2} is a side chain of an amino acid. In some embodiments, R^{a2} is a side chain of a standard amino acid.

In some embodiments, R^{a3} is -L^{a}-R, wherein R is as described in the present disclosure. In some embodiments, R^{a3} is -L^{a}-R, wherein R is an optionally substituted group selected from C₃₋₃₀ cycloaliphatic, C₅₋₃₀ aryl, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon. In some embodiments, R^{a3} is -L^{a}-R, wherein R is an optionally substituted group selected from C₆₋₃₀ aryl and 5-30 membered heteroaryl having 1-10 heteroatoms independently selected from oxygen, nitrogen, sulfur, phosphorus and silicon. In some embodiments, R^{a3} is a side chain of an amino acid. In some embodiments, R^{a3} is a side chain of a standard amino acid.

In some embodiments, one or R^{a2} and R^{a3} is —H. In some embodiments, one or R^{a2} and R^{a3} is -L^{a}-R, wherein L^{a} is as described herein. In some embodiments, L^{a} is not a covalent bond. In some embodiments, one or more methylene units of L^{a} are independently and optionally replaced as described herein, e.g., with —C(O)—, —N(R′)—, —O—, —C(O)—N(R′)— and/or -Cy-, etc. In some embodiments, L^{a} is or comprises —C(O)—, —N(R′)— and -Cy-. In some embodiments, L^{a} is or comprises —C(O)N(R′)— and -Cy-. In some embodiments, as described herein, -Cy- is substituted and one or more substituents are independently an electron-withdrawing group.

In some embodiments, an amino acid side chain is R^{a2} or R^{a3}. In some embodiments, an amino acid side chain is or comprises -L^{LG1}-L^{LG2}-L^{LG3}-L^{LG4}-H. In some embodiments, an amino acid side chain is or comprises -L^{LG2}-L^{LG3}-L^{LG4}-H. In some embodiments, an amino acid side chain is or comprises -L^{LG3}-L^{LG4}-H. In some embodiments, an amino acid side chain is or comprises -L^{LG4}-H. In some embodiments, such a side chain is

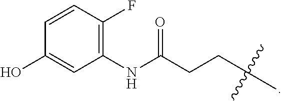 [SMILES: *CCC(=O)Nc1cc(O)ccc1F]

In some embodiments, such a side chain is

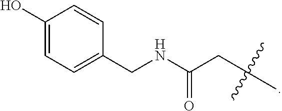 [SMILES: *CC(=O)NCc1ccc(O)cc1]

In some embodiments, such a side chain is

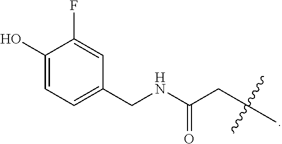 [SMILES: *CC(=O)NCc1ccc(O)c(F)c1]

In some embodiments, such a side chain is

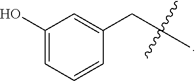 [SMILES: *Cc1cccc(O)c1]

In some embodiments, R is an optionally substituted C₁₋₆ aliphatic. In some embodiments, R is an optionally substituted C₁₋₆ alkyl. In some embodiments, R is —CH₃. In some embodiments, R is optionally substituted pentyl. In some embodiments, R is n-pentyl.

In some embodiments, R is a cyclic group. In some embodiments, R is an optionally substituted C₃₋₃₀ cycloaliphatic group. In some embodiments, R is cyclopropyl.

In some embodiments, R is an optionally substituted aromatic group, and an amino acid residue of an amino acid of formula A-I is a Xaa^{A}. In some embodiments, R^{a2} or R^{a3} is —CH₂—R, wherein R is an optionally substituted aryl or heteroaryl group. In some embodiments, R is optionally substituted phenyl. In some embodiments, R is phenyl. In some embodiments, R is optionally substituted phenyl. In some embodiments, R is 4-trifluoromethylphenyl. In some embodiments, R is 4-phenylphenyl. In some embodiments, R is optionally substituted 5-30 membered heteroaryl having 1-10 heteroatoms independently selected from oxygen, nitrogen, sulfur, phosphorus and silicon. In some embodiments, R is optionally substituted 5-14 membered heteroaryl having 1-5 heteroatoms independently selected from oxygen, nitrogen, and sulfur. In some embodiments, R is

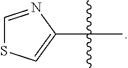 [SMILES: *c1cscn1]

In some embodiments, R is optionally substituted pyridinyl. In some embodiments, R is 1-pyridinyl. In some embodiments, R is 2-pyridinyl. In some embodiments, R is 3-pyridinyl. In some embodiments, R is

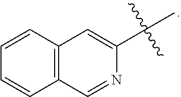 [SMILES: *c1cc2ccccc2cn1]

In some embodiments, R′ is-COOH. In some embodiments, a compound of and an amino acid residue of an amino acid of formula A-I is a Xaa^{N}.

In some embodiments, R′ is —NH₂. In some embodiments, a compound of an amino acid residue of an amino acid of formula A-I is a Xaa^{p}.

In some embodiments, R^{a2} or R^{a3} is R, wherein R is C₁₋₂₀ aliphatic as described in the present disclosure. In some embodiments, a compound of an amino acid residue of an amino acid of formula A-I is a Xaa^{H}. In some embodiments, R is —CH₃. In some embodiments, R is ethyl. In some embodiments, R is propyl. In some embodiments, R is n-propyl. In some embodiments, R is butyl. In some embodiments, R is n-butyl. In some embodiments, R is pentyl. In some embodiments, R is n-pentyl. In some embodiments, R is cyclopropyl.

In some embodiments, two or more of R^{a1}, R^{a2}, and R^{a3} are R and are taken together to form an optionally substituted ring as described in the present disclosure.

In some embodiments, R^{a1} and one of R^{a2} and R^{a3} are R and are taken together to form an optionally substituted 3-6 membered ring having no additional ring heteroatom other than the nitrogen atom to which R^{a1} is bonded to. In some embodiments, a formed ring is a 5-membered ring as in proline.

In some embodiments, R^{a2} and R^{a3} are R and are taken together to form an optionally substituted 3-6 membered ring as described in the present disclosure. In some embodiments, R^{a2} and R^{a3} are R and are taken together to form an optionally substituted 3-6 membered ring having one or more nitrogen ring atom. In some embodiments, R^{a2} and R^{a3} are R and are taken together to form an optionally substituted 3-6 membered ring having one and no more than one ring heteroatom which is a nitrogen atom. In some embodiments, a ring is a saturated ring.

In some embodiments, an amino acid is a natural amino acid. In some embodiments, an amino acid is an unnatural amino acid. In some embodiments, an amino acid is an alpha-amino acid. In some embodiments, an amino acid is a beta-amino acid. In some embodiments, a compound of formula A-I is a natural amino acid. In some embodiments, a compound of formula A-I is an unnatural amino acid.

In some embodiments, an amino acid comprises a hydrophobic side chain. In some embodiments, an amino acid with a hydrophobic side chain is A, V, I, L, M, F, Y or W. In some embodiments, an amino acid with a hydrophobic side chain is A, V, I, L, M, or F. In some embodiments, an amino acid with a hydrophobic side chain is A, V, I, L, or M. In some embodiments, an amino acid with a hydrophobic side chain is A, V, I, or L. In some embodiments, a hydrophobic side chain is R wherein R is C₁₋₁₀ aliphatic. In some embodiments, R is C₁₋₁₀ alkyl. In some embodiments, R is methyl. In some embodiments, R is ethyl. In some embodiments, R is propyl. In some embodiments, R is butyl. In some embodiments, R is pentyl. In some embodiments, R is n-pentyl. In some embodiments, an amino acid with a hydrophobic side chain is NH₂CH(CH₂CH₂CH₂CH₂CH₃)COOH. In some embodiments, an amino acid with a hydrophobic side chain is (S)—NH₂CH(CH₂CH₂CH₂CH₂CH₃)COOH. In some embodiments, an amino acid with a hydrophobic side chain is (R)—NH₂CH(CH₂CH₂CH₂CH₂CH₃)COOH. In some embodiments, a hydrophobic side chain is —CH₂R wherein R is optionally substituted phenyl. In some embodiments, R is phenyl. In some embodiments, R is phenyl substituted with one or more hydrocarbon group. In some embodiments, R is 4-phenylphenyl. In some embodiments, an amino acid with a hydrophobic side chain is NH₂CH(CH₂₋₄-phenylphenyl)COOH. In some embodiments, an amino acid with a hydrophobic side chain is (S)—NH₂CH(CH₂₋₄-phenylphenyl)COOH. In some embodiments, an amino acid with a hydrophobic side chain is (R)—NH₂CH(CH₂₋₄-phenylphenyl)COOH.

In some embodiments, an amino acid comprises a positively charged side chain (e.g., at physiological pH) as described herein. In some embodiments, such an amino acid comprises a basic nitrogen in its side chain. In some embodiments, such an amino acid is Arg, His or Lys. In some embodiments, such an amino acid is Arg. In some embodiments, such an amino acid is His. In some embodiments, such an amino acid is Lys.

In some embodiments, an amino acid comprises a negatively charged side chain (e.g., at physiological pH) as described herein. In some embodiments, such an amino acid comprises a —COOH in its side chain. In some embodiments, such an amino acid is Asp. In some embodiments, such an amino acid is Glu.

In some embodiments, an amino acid comprises a side chain comprising an aromatic group as described herein. In some embodiments, such an amino acid is Phe, Tyr, Trp, or His. In some embodiments, such an amino acid is Phe. In some embodiments, such an amino acid is Tyr. In some embodiments, such an amino acid is Trp. In some embodiments, such an amino acid is His. In some embodiments, such an amino acid is NH₂—CH(CH₂₋₄-phenylphenyl)-COOH. In some embodiments, such an amino acid is (S)—NH₂—CH(CH₂₋₄-phenylphenyl)-COOH. In some embodiments, such an amino acid is (R)—NH₂—CH(CH₂₋₄-phenylphenyl)-COOH.

In some embodiments, an amino acid is

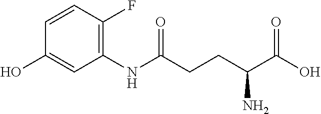 [SMILES: N[C@@H](CCC(=O)Nc1cc(O)ccc1F)C(=O)O]

or a salt thereof. In some embodiments, an amino acid is

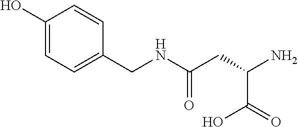 [SMILES: N[C@@H](CC(=O)NCc1ccc(O)cc1)C(=O)O]

or a salt thereof. In some embodiments, an amino acid is

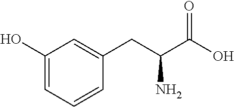 [SMILES: N[C@@H](Cc1cccc(O)c1)C(=O)O]

or a salt thereof. In some embodiments, an amino acid is

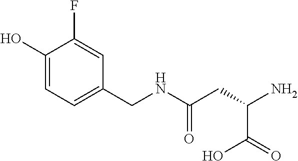 [SMILES: N[C@@H](CC(=O)NCc1ccc(O)c(F)c1)C(=O)O]

or a salt thereof. In some embodiments, an amino acid is

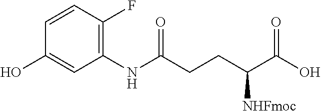

or a salt thereof. In some embodiments, an amino acid is

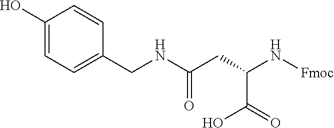

or a salt thereof. In some embodiments, an amino acid is

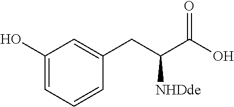

or a salt thereof. In some embodiments, an amino acid is

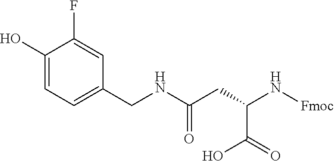

or a salt thereof. In some embodiments, a provided compound is

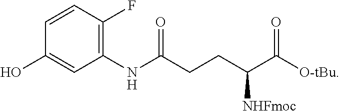

In some embodiments, the present disclosure provides polypeptide agents comprising one or more amino acid residues described in the present disclosure.

### Target

In some embodiments, the present disclosure provides technologies for selectively directing agents comprising target binding moieties (e.g. ARM agents, MATE agents, etc.) and/or antibodies (and optionally immune cells recruited by antibodies, e.g., NK cells) to desired target sites comprising one or more targets. As those skilled in the art will appreciate, provided technologies are useful for various types of targets, particularly those comprising components of SARS-CoV-2, e.g. SARS-CoV-2 viruses, cells infected thereby, cells expressing a SARS-CoV-2 spike protein or a fragment thereof, etc.

In some embodiments, targets are damaged or defective tissues. In some embodiments, a target is a damaged tissue. In some embodiments, a target is a defective tissue. In some embodiments, a target is associated with a disease, disorder or condition, e.g., COVID-19. In some embodiments, targets are or comprise diseased cells. In some embodiments, targets are or comprise cells infected by SARS-CoV-2 viruses. In some embodiments, a target is a foreign object. In some embodiments, a target is or comprises an infectious agent, e.g., a SARS-CoV-2 virus. In some embodiments, a target is or comprises viruses, e.g. SARS-CoV-2 viruses. In some embodiments, targets comprise or express a SARS-CoV-2 spike protein or a fragment thereof.

### General Methods of Providing Various Agents

Agents of the present disclosure may be prepared or isolated in general by synthetic and/or semi-synthetic methods or recombinant methods in accordance with the present disclosure. Certain technologies are described in the Examples. In some embodiments, polypeptide agents, e.g., target binding moiety peptide agents, maybe be prepared using biological expression systems. In some embodiments, provided agents are prepared synthetically. In some embodiments, provided agents are prepared using certain technologies described in WO 2019/023501.

Various technologies, e.g., those for preparing antibody-drug conjugates, may be utilized in preparation of MATE agents. In many such technologies, conjugation is not selective with respect to amino acid residue sites, and product compositions usually contain various different types of agents which may differ from each other with respect to number of target binding moieties conjugated and/or conjugation sites. In some embodiments, the present disclosure provides technologies that can be utilized for selective conjugation of target binding moieties at particular amino acid residue sites.

In some embodiments, the present disclosure provides a method, comprising:
contacting a first agent comprising a target binding moiety linked to a first reactive group optionally through a first linker with a second agent comprising an antibody moiety linked to a second reactive group optionally through a second linker, wherein the first reactive group reacts with a second reactive group, and
forming a product agent comprising a target binding moiety and an antibody binding moiety optionally through a linker.

In some embodiments, the present disclosure provides a method comprising:
contacting a first composition comprising a plurality of first agents each independently comprising a target binding moiety linked to a first reactive group optionally through a first linker moiety with a second composition comprising a plurality of second agents each independently comprising an antibody moiety optionally linked to a second reactive group optionally through a second linker moiety,
wherein a product composition comprising a plurality of product agents each independently comprising a target binding moiety and an antibody binding moiety optionally through a linker is formed.

In some embodiments, a first composition is a composition comprising a first agent as described herein. In some embodiments, second agents independently comprise second reactive groups. In some embodiments, a second composition is a composition comprising a plurality of agents as described herein, wherein each moiety of interest is independently a reactive group as described herein. In some embodiments, a second composition is an antibody composition, wherein antibodies in the composition are not chemically modified. In some embodiments, a second composition is an IVIG preparation. In some embodiments, a product composition is a composition comprising a plurality of agents as described herein, wherein each moiety of interest is independently a target binding moiety as described herein.

In some embodiments, a target binding moiety in a product agent is a target binding moiety in a first agent. In some embodiments, an antibody moiety in a product agent is an antibody moiety in a second agent. In some embodiments, a second agent is an antibody agent, e.g., a monoclonal antibody, an antibody in a polyclonal antibody, an antibody in an IVIG preparation, etc. In some embodiments, a second reactive group is a function group of an amino acid residue, e.g., —NH₂ of Lys, —SH of Cys, etc. In some embodiments, a second reactive group is —NH₂ of a Lys residue, e.g., of a residue selected from K246 and K248 of an IgG1 heavy chain amino acid residues corresponding thereto, K251 and K253 of an IgG2 heavy chain and amino acid residues corresponding thereto, and K239 and K241 of an IgG4 heavy chain and amino acid residues corresponding thereto. In some embodiments, the present disclosure provides selective reactions at particular amino acid residues of antibody moieties.

In some embodiments, a second reactive group is installed to an antibody moiety optionally through a linker. In some embodiments, a second reactive group is installed to an antibody moiety through a linker. In some embodiments, a second reactive group is selectively linked to certain location(s) of an antibody moiety, e.g., certain location(s) selected from K246 and K248 of an IgG1 heavy chain amino acid residues corresponding thereto, K251 and K253 of an IgG2 heavy chain and amino acid residues corresponding thereto, and K239 and K241 of an IgG4 heavy chain and amino acid residues corresponding thereto. In some embodiments, the present disclosure provides selective reactions at particular amino acid residues of antibody moieties.

In some embodiments, the present disclosure provides agents each independently comprising an antibody binding moiety that binds to an antibody agent, a reactive group, a moiety of interest, and optionally one or more linker moieties linking such groups/moieties. In some embodiments, such agents are useful as reaction partners (e.g., first agents) for conjugating moieties of interest, e.g., target binding moieties, reactive groups (e.g., second reactive groups) to agents comprising antibody moieties (e.g., second agents). In some embodiments, the present disclosure provides agents for conjugating moieties of interest to antibody moieties in various agents or antibody agents (e.g., monoclonal antibody agents, polyclonal antibody agents, antibody agents of IVIG preparations, etc.). In some embodiments, provided agents each comprise a moiety of interest, a reactive group, an antibody binding moiety, and optionally one or more linker moieties (linkers) linking such moieties. In some embodiments, an antibody binding moiety is part of a leaving group that is released upon contacting such an agent (e.g., a first agent) with an antibody moiety (e.g., of a second agent) and reacting a reactive group of such an agent (e.g., a first reactive group of a first agent) with a reactive group of an antibody moiety (e.g., a second reactive group of a second agent, such as —NH₂ of a Lys residue of an antibody protein). In some embodiments, provided technologies among other things can provide improved conjugation efficiency, high selectivity, and/or fewer steps (in some cases, single step) to conjugation product agents. In some embodiments, a provided agent, e.g., a first agent, is a compound of formula R-I or a salt thereof:

LG-RG-L^{RM}-MOI, (R-I)

or a salt thereof, wherein:
LG is a group comprising an antibody binding moiety;
RG is a reactive group;
L^{RM} is a linker; and
MOI is a moiety of interest.

In some embodiments, LG is or comprises an antibody binding moiety as described herein, and a linker which links an antibody binding moiety and RG.

As used in the present disclosure, a moiety generally refers to a part of a molecule, e.g., in an ester RCOOR′, the alcohol moiety is RO—. In some embodiments, a moiety of an agent (e.g., a target agent, a peptide agent, an antibody agent, etc.) retains one or more or all desirable structural features, properties, functions, and/or activities of a compound. For example, in some embodiments, a target binding moiety can bind to a target, optionally in a comparable fashion, as its corresponding target binding agent; in some embodiments, a target agent moiety maintains one or more desired structural features, properties, functions, and/or properties comparable to its corresponding target agent; in some embodiments, an antibody agent moiety maintains one or more desired structural features, properties, functions, and/or properties (e.g., 3-dimension structure, antigen specificity, antigen-binding capacity, and/or immunological functions, etc.) comparable to its corresponding antibody agent. In some embodiments, a moiety of an agent, e.g., a target agent moiety, a peptide agent moiety, an antibody agent moiety, etc. is a monovalent (for a monovalent moiety), bivalent (for a bivalent moiety), or polyvalent (for a polyvalent moiety) radical of an agent, e.g., a target agent (for a target agent moiety), a peptide agent (for a peptide agent moiety), an antibody agent (for an antibody agent moiety), etc. In some embodiments, a monovalent radical is formed by removing a monovalent part (e.g., hydrogen, halogen, another monovalent group like alkyl, aryl, etc.) from a compound/agent. In some embodiments, a bivalent or polyvalent radical is formed by removing one or more monovalent (e.g., hydrogen, halogen, monovalent groups like alkyl, aryl, etc.), bivalent and/or polyvalent parts from a compound/agent. In some embodiments, radicals are formed by removing hydrogen atoms. In some embodiments, a moiety is monovalent. In some embodiments, a moiety is bivalent. In some embodiments, a moiety is polyvalent.

In some embodiments, LG is or comprises R^{LG}-L^{LG}-, wherein R^{LG} is or comprises an antibody binding moiety, and L^{LG} is a linker moiety as described herein. In some embodiments, LG is ABT-L^{LG}-. In some embodiments, L^{LG} is -L^{LG1}-L^{LG2}-, wherein each of L^{LG1} and L^{LG2} is independently a linker moiety as described herein. In some embodiments, L^{LG} is -L^{LG1}-L^{LG2}-L^{LG3}-, wherein each of L^{LG1}, L^{LG2} and L^{LG3} is independently as linker moiety described herein. In some embodiments, L^{LG} is L^{LG1}-L^{LG2}-L^{LG3}-L^{LG4}-, wherein each of L^{LG1}, L^{LG2}, L^{LG3} and L^{LG4} is independently a linker moiety as described herein. In some embodiments, L^{LG1} is bonded to R^{LG}. In some embodiments, L^{LG1} is bonded to moiety of interest. In some embodiments, L^{LG} is -L^{LG1}-, and a reactive group comprises L^{LG2}, L^{LG3} and L^{LG4}. In some embodiments, L^{LG} is -L^{LG1}-L^{LG2}-, and a reactive group comprises L^{LG3} and L^{LG4} In some embodiments, L^{LG} is -L^{LG1}-L^{LG2}-L^{LG3}-, and a reactive group comprises L^{LG4} In some embodiments, each of L^{LG1}, L^{LG2}, L^{LG3} and L^{LG4} is independently L as described herein.

In some embodiments, antibody binding moieties, LG, etc. are released after reactions, e.g., after first agents (e.g., wherein MOIs are target binding moieties) react with second agents (e.g., which are antibody agents comprising reactive amino acid residues such as amino groups as second reactive groups and/or second agents comprising second reactive groups introduced to antibody agents), or after first agents (e.g., wherein MOIs are reactive groups such as second reactive groups) react with second agents which are antibody agents. In some embodiments, an antibody binding moiety is released after a reaction. In some embodiments, LG is released after a reaction. In some embodiments, a leaving group is released as part of a compound having the structure of LG-H or a salt thereof. In some embodiments, an antibody binding moiety is released as part of a compound having the structure of LG-H or a salt thereof. In some embodiments, LG is released as part of a compound having the structure of LG-H or a salt thereof. In some embodiments, a released compound has the structure of R^{LG}-L^{LG1}-L^{LG2}-L^{LG3}-L^{LG4}-H or a salt thereof. In some embodiments, an antibody binding moiety is released as part of a compound having the structure of R^{LG}-L^{LG1}-L^{LG2}-L^{LG3}-L^{LG4}-H or a salt thereof. In some embodiments, an antibody binding moiety is released as part of a compound having the structure of R^{LG}-L^{LG1}L^{LG2}-L^{LG3}-L^{LG4}-H or a salt thereof, wherein R^{LG} is or comprises an antibody binding moiety. In some embodiments, LG is released as part of a compound having the structure of R^{LG}-L^{LG1}L^{LG2}-L^{LG3}-L^{LG4}-H or a salt thereof, wherein LG is R^{LG}-L^{LG}, and L^{LG} is -L^{LG1}-, -L^{LG1}-L^{LG2}-, -L^{LG1}-L^{LG2}-L^{LG3}-, or -L^{LG1}-L^{LG2}-L^{LG3}-L^{LG4}-. In some embodiments, LG is released as part of a compound having the structure of R^{LG}-L^{LG1}-L^{LG2}-L^{LG3}-L^{LG4}-H or a salt thereof, wherein LG is R^{LG}-L^{LG1}-. In some embodiments, LG is released as part of a compound having the structure of R^{LG}-L^{LG1}L^{LG2}-L^{LG3}-L^{LG4}-H or a salt thereof, wherein LG is R^{LG}-L^{LG1}-L^{LG2} In some embodiments, LG is released as part of a compound having the structure of R^{LG}-L^{LG1}-L^{LG2}-L^{LG3}-L^{LG4}-H or a salt thereof, wherein LG is R^{LG}-L^{LG1}-L^{LG2}-L^{LG3}. In some embodiments, LG is released as part of a compound having the structure of R^{LG}-L^{LG1}-L^{LG2}-L^{LG3}-L^{LG4}-H or a salt thereof, wherein LG is R^{LG}-L^{LG1}-L^{LG2}-L^{LG3}-L^{LG4}.

In some embodiments, L is a covalent bond, or a bivalent optionally substituted, linear or branched C₁₋₁₀₀ group comprising one or more aliphatic moieties, aryl moieties, heteroaliphatic moieties each independently having 1-20 heteroatoms, heteroaromatic moieties each independently having 1-20 heteroatoms, or any combinations of any one or more of such moieties, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or —[(—O—C(R′)₂—C(R′)₂—)n]-, wherein n is 1-20. In some embodiments, L is a covalent bond, or a bivalent optionally substituted, linear or branched C₁₋₁₀₀ aliphatic or heteroaliphatic group 1-20 heteroatoms, wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, or —[(—O—C(R′)₂—C(R′)₂—)ₙ]—, wherein n is 1-20. In some embodiments, L is a covalent bond, or a bivalent optionally substituted, linear or branched C₁, C₂, C₃, C₄, C₅, C₁₀, C₁₅, C₂₀, C₂₅, C₃₀, C₄₀, C₅₀, C₆₀, C₁₋₂, C₁₋₅, C₁₋₁₀, C₁₋₁₅, C₁₋₂₀, C₁₋₃₀, C₁₋₄₀, C₁₋₅₀, C₁₋₆₀, C₁₋₇₀, C₁₋₈₀, or C₁₋₉₀ aliphatic or heteroaliphatic group 1-10 heteroatoms, wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, amino acid residues, or —[(—O—C(R′)₂—C(R′)₂—)ₙ]—, wherein n is 1-20. In some embodiments, L is a covalent bond, or a bivalent optionally substituted, linear or branched C₁, C₂, C₃, C₄, C₅, C₁₀, C₁₅, C₂₀, C₂₅, C₃₀, C₄₀, C₅₀, C₆₀, C₁₋₂, C₁₋₅, C₁₋₁₀, C₁₋₁₅, C₁₋₂₀, C₁₋₃₀, C₁₋₄₀, C₁₋₅₀, C₁₋₆₀, C₁₋₇₀, C₁₋₈₀, or C₁₋₉₀ aliphatic or heteroaliphatic group 1-10 heteroatoms, wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, amino acid residues, or —[(—O—C(R′)₂—C(R′)₂—)ₙ]—, wherein n is 1-10. In some embodiments, L is a covalent bond, or a bivalent optionally substituted, linear or branched C₁, C₂, C₃, C₄, C₅, C₁₀, C₁₅, C₂₀, C₂₅, C₃₀, C₄₀, C₅₀, C₆₀, C₁₋₂, C₁₋₅, C₁₋₁₀, C₁₋₁₅, C₁₋₂₀, C_{1- 30}, C₁₋₄₀, C₁₋₅₀, C₁₋₆₀, C₁₋₇₀, C₁₋₈₀, or C₁₋₉₀ aliphatic group, wherein one or more methylene units of the group are optionally and independently replaced with —O—, —N(R′)—, —C(O)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, amino acid residues, or —[(—O—C(R′)₂—C(R′)₂—)ₙ]—, wherein n is 1-10. In some embodiments, L is a covalent bond, or a bivalent optionally substituted, linear or branched C₁, C₂, C₃, C₄, C₅, C₁₀, C₁₅, C₂₀, C₂₅, C₃₀, C₄₀, C₅₀, C₆₀, C₁₋₂, C₁₋₅, C₁₋₁₀, C₁₋₁₅, C₁₋₂₀, C₁₋₃₀, C₁₋₄₀, C₁₋₅₀, C₁₋₆₀, C₁₋₇₀, C₁₋₈₀, or C₁₋₉₀ aliphatic group, wherein one or more methylene units of the group are optionally and independently replaced with —O—, —N(R′)—, —C(O)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, or —[(—O—C(R′)₂—C(R′)₂—)ₙ]—, wherein n is 1-10. In some embodiments, L is a covalent bond, or a bivalent optionally substituted, linear or branched C₁₋₁₀ aliphatic group, wherein one or more methylene units of the group are optionally and independently replaced with —O—, —N(R′)—, —C(O)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, -Cy-, or —[(—O—C(R′)₂—C(R′)₂—),]-, wherein n is 1-10. In some embodiments, L is a covalent bond, or a bivalent optionally substituted, linear or branched C₁₋₁₀ aliphatic group, wherein one or more methylene units of the group are optionally and independently replaced with —O—, —N(R′)—, —C(O)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, or —[(—O—C(R′)₂—C(R′)₂—)ₙ]—, wherein n is 1-10. In some embodiments, L comprises no —C(O)O—. In some embodiments, L comprises no —C(O)—N(R′)—. In some embodiments, L comprises no —S—. In some embodiments, L comprises no —S-Cy-. In some embodiments, L comprises no —S—S—. In some embodiments, L does not contain one or more or any of —C(O)O—, —C(O)—N(R′)—, —S—, and —S—S—. In some embodiments, L does not contain one or more or any of —C(O)O—, —C(O)—N(R′)—, —S-Cy-, and —S—S—. In some embodiments, L does not contain one or more or any of —C(O)O—, —S—, and —S—S—. In some embodiments, L does not contain one or more or any of —C(O)O—, —S-Cy-, and —S—S—. In some embodiments, L contains none of —C(O)O—, —S—, and —S—S—. In some embodiments, L contains none of —C(O)O—, —S-Cy-, and —S—S—. In some embodiments, L contains none of —C(O)O— and —S—S—.

In some embodiments, L is a covalent bond. In some embodiments, L is not a covalent bond.

In some embodiments, L^{LG1} is a covalent bond. In some embodiments, L^{LG1} is not a covalent bond. In some embodiments, L^{LG1} is or comprises —(CH₂CH₂O)n-. In some embodiments, L^{LG1} is or comprises —(CH₂)n-O—(CH₂CH₂O)n-(CH₂)n-, wherein each n is independently as described herein, and each —CH₂— is independently optionally substituted. In some embodiments, L^{LG1} is —(CH₂)n-O—(CH₂CH₂O)n-(CH₂)n-, wherein each n is independently as described herein, and each —CH₂— is independently optionally substituted. In some embodiments, L^{LG1} is —(CH₂)₂—O—(CH₂CH₂O)n-(CH₂)₂—, wherein n is as described herein, and each —CH₂— is independently optionally substituted. In some embodiments, L^{LG1} is —(CH₂)₂—O—(CH₂CH₂O)n-(CH₂)₂—, wherein n is as described herein.

In some embodiments, L^{LG1} is —CH₂—. In some embodiments, L^{LG1} is —(CH₂)₂—. In some embodiments, L^{LG1} is —(CH₂)₂—C(O)—. In some embodiments, L^{LG1} is —(CH₂)₂—C(O)—NH—. In some embodiments, L^{LG1} is —(CH₂)₃—. In some embodiments, L^{LG1} is —(CH₂)₃NH—. In some embodiments, L^{LG1} is —(CH₂)₃NH—C(O)—. In some embodiments, L^{LG1} is —C(O)—(CH₂)₃NH—C(O)—. In some embodiments, L^{LG1} is —C(O)—(CH₂)₃—. In some embodiments, L^{LG1} is —NH—C(O)—(CH₂)₃—. In some embodiments, L^{LG1} is —NHC(O)—(CH₂)₃NH—C(O)—. In some embodiments, a —CH₂— is bonded to an antibody binding moiety.

In some embodiments, L^{LG1} is —CH₂CH₂—O—CH₂CH₂—O—CH₂CH₂—. In some embodiments, L^{LG1} is —CH₂CH₂—O—CH₂CH₂—O—CH₂CH₂—C(O)—. In some embodiments, L^{LG1} is —CH₂CH₂—O—CH₂CH₂—O—CH₂CH₂—C(O)NH—. In some embodiments, L^{LG1} is —CH₂CH₂—O—CH₂CH₂—O—CH₂CH₂—C(O)NH—CH₂—. In some embodiments, —CH₂CH₂— is bonded to an antibody binding moiety.

In some embodiments, L^{LG1} is —(CH₂CH₂O)n-. In some embodiments, L^{LG1} is —(CH₂CH₂O)n-CH₂—CH₂—. In some embodiments, L^{LG1} is —(CH₂CH₂O)n-CH₂—CH₂—C(O)—. In some embodiments, L^{LG1} is —(CH₂CH₂O)₂—CH₂—CH₂—C(O)—. In some embodiments, L^{LG1} is —(CH₂CH₂O)₄—CH₂—CH₂—C(O)—. In some embodiments, L^{LG1} is —(CH₂CH₂O)₈—CH₂—CH₂—C(O)—. In some embodiments, —C(O)— is bonded to an antibody binding moiety.

In some embodiments, L^{LG1} is —N(R′)—. In some embodiments, L^{LG1} is —NH—. In some embodiments, L^{LG1} is —NH—[(—CH₂CH₂—O—)]n-. In some embodiments, L^{LG1} is —NH—[(—CH₂CH₂—O—)]n-CH₂CH₂—. In some embodiments, L^{LG1} is —NH—[(—CH₂CH₂—O—)]n-CH₂CH₂—NH—. In some embodiments, L^{LG1} is —NH—[(—CH₂CH₂—O—)]n-CH₂CH₂—NH—C(O)—. In some embodiments, n is 1. In some embodiments, n is 2. In some embodiments, n is 3. In some embodiments, n is 4. In some embodiments, n is 5. In some embodiments, L^{LG1} is —NH—CH₂CH₂—O—. In some embodiments, L^{LG1} is —NH—CH₂CH₂—O—CH₂CH₂—. In some embodiments, L^{LG1} is —NH—CH₂CH₂—O—CH₂CH₂—NH—. In some embodiments, L^{LG1} is —NH—CH₂CH₂—O—CH₂CH₂—NH—C(O)—.

In some embodiments, L^{LG1} is —NH—[(—CH₂CH₂—O—)]₂—. In some embodiments, L^{LG1} is —NH—[(—CH₂CH₂—O—)]₂—CH₂CH₂—. In some embodiments, L^{LG1} is —NH—[(—CH₂CH₂—O—)]₂—CH₂CH₂—NH—. In some embodiments, L^{LG1} is —NH—[(—CH₂CH₂—O—)]₂—CH₂CH₂—NH—C(O)—.

In some embodiments, L^{LG1} is —NH—[(—CH₂CH₂—O—)]₃—. In some embodiments, L^{LG1} is —NH—[(—CH₂CH₂—O—)]₃—CH₂CH₂—. In some embodiments, L^{LG1} is —NH—[(—CH₂CH₂—O—)]₃—CH₂CH₂—NH—. In some embodiments, L^{LG1} is —NH—[(—CH₂CH₂—O—)]₃—CH₂CH₂—NH—C(O)—. In some embodiments L^{G1} is —NH—[(—CH₂⁻CH₂—O—)]₂—C(O)—NH. In some embodiments, L^{LG1} is —NH—[(—CH₂CH₂—O—)]₄—. In some embodiments, L^{LG1} is —NH—[(—CH₂CH₂—O—)]₄—CH₂CH₂—. In some embodiments, L^{LG1} is —NH—[(—CH₂CH₂—O—)]₄—CH₂CH₂—NH—. In some embodiments, L^{LG1} is —NH—[(—CH₂CH₂—O—)]₄—CH₂CH₂—NH—C(O)—. In some embodiments, L^{LG1} is —NH—[(—CH₂CH₂—O—)]₅—. In some embodiments, L^{LG1} is —NH—[(—CH₂CH₂—O—)]₅—CH₂CH₂—. In some embodiments, L^{LG1} is —NH—[(—CH₂CH₂—O—)]₅—CH₂CH₂—NH—. In some embodiments, L^{LG1} is —NH—[(—CH₂CH₂—O—)]₅—CH₂CH₂—NH—C(O)—. In some embodiments, —NH— is bonded to an antibody binding moiety.

In some embodiments, L^{LG1} is —CH₂—. In some embodiments, L^{LG1} is —CH₂CH₂—. In some embodiments, L^{LG1} is —CH₂CH₂NH—. In some embodiments, L^{LG1} is —CH₂CH₂NH—(CO)—. In some embodiments, —CH₂— is bonded to an antibody binding moiety.

In some embodiments, L^{LG1} is —CH₂—. In some embodiments, L^{LG1} is —CH₂C(O)—. In some embodiments, L^{LG1} is —CH₂C(O)NH—. In some embodiments, L^{LG1} is —CH₂(CO)NHCH₂—. In some embodiments, —CH₂—C(O)— is bonded to an antibody binding moiety at —CH₂—.

In some embodiments, L^{LG2} is a covalent bond. In some embodiments, L^{LG2} is not a covalent bond. In some embodiments, L^{LG2} is —N(R′)C(O)—. In some embodiments, L^{LG2} is —NHC(O)—. In some embodiments, L^{LG2} is —(CH₂)n-N(R′)C(O)—, wherein —(CH₂)n- is optionally substituted. In some embodiments, L^{LG2} is —(CH₂)n-OC(O)—, wherein —(CH₂)n- is optionally substituted. In some embodiments, L^{LG2} is —(CH₂)n-OC(O)N(R′)—, wherein —(CH₂)n- is optionally substituted. In some embodiments, L^{LG2} is —(CH₂)n-OC(O)NH—, wherein —(CH₂)n- is optionally substituted. In some embodiments, n is 1-10, e.g., 1, 2, 3, 4, 5, 6, 7, 8, 9, or 10. In some embodiments, n is 1. In some embodiments, n is 2. In some embodiments, n is 3. In some embodiments, —(CH₂)n- is substituted. In some embodiments, —(CH₂)n- is unsubstituted. In some embodiments, L^{LG2} is —CH₂N(CH₂CH₂CH₂S(O)₂OH)—C(O)—. In some embodiments, L^{LG2} is —C(O)—NHCH₂—. In some embodiments, L^{LG2} is —C(O)—NHCH₂CH₂—. In some embodiments, L^{LG2} is —C(O)O—CH₂—. In some embodiments, L^{LG2} is —NH—C(O)O—CH₂—. In some embodiments, —C(O)— is bonded to L^{LG3} In some embodiments, —N(R′)—, —NH—, or an optionally substituted —CH₂— unit (of optionally substituted —(CH₂)n-) is bonded to L^{LG3}. In some embodiments L^{LG2} is —NH—, —NHC(O)—, —(CH₂)n-NHC(O)—, —(CH₂)n-OC(O)—, —(CH₂)n-OC(O)NH—, —C(O)—NHCH₂—, —C(O)—NHCH₂CH₂—, —C(O)O—CH₂—, or —NH—C(O)O—CH₂—.

In some embodiments, L^{LG2} is —N(R′)—. In some embodiments, L^{LG2} is —N(R)—. In some embodiments, L^{LG2} is —NH—.

In some embodiments, L^{LG2} is optionally substituted bivalent C₁₋₆ aliphatic. In some embodiments, L^{LG2} is —CH₂—. In some embodiments, L^{LG2} is —CH₂NH—. In some embodiments, L^{LG2} is —CH₂NH—C(O)—. In some embodiments, L^{LG2} is —CH₂NH—C(O)—CH₂—.

In some embodiments, L^{LG3} is or comprises an optionally substituted aryl ring. In some embodiments, L^{LG3} is or comprises an optionally substituted phenyl ring. In some embodiments, L^{LG3} is a phenyl ring substituted with one or more electron-withdrawing groups. As appreciated by those skilled in the art, various electron-withdrawing groups are known in the art and may be utilized in accordance with the present disclosure. In some embodiments, an electron-withdrawing group is halogen. In some embodiments, an electron-withdrawing group is —F. In some embodiments, it is —Cl. In some embodiments, it is —Br. In some embodiments, it is —I. In some embodiments, an electron-withdrawing group comprises an X═Y double bond, wherein X is bonded to the group to which the electron-withdrawing group is a substituent, and at least one of X and Y is a heteroatom. In some embodiments, X is a heteroatom. In some embodiments, Y is a heteroatom. In some embodiments, each of X and Y is independently a heteroatom. In some embodiments, Y is O. In some embodiments, Y is S. In some embodiments, X is C. In some embodiments, X is N. In some embodiments, X is P. In some embodiments, X is S. In some embodiments, X═Y is C═O. In some embodiments, X═Y is N═O. In some embodiments, X═Y is S═O. In some embodiments, X═Y is P═O. In some embodiments, an electron-withdrawing group is —C(O)-L-R′. In some embodiments, an electron-withdrawing group is —C(O)—R′. In some embodiments, it is —NO₂. In some embodiments, it is —S(O)-L-R′. In some embodiments, it is —S(O)—R′. In some embodiments, it is —S(O)₂-L-R′. In some embodiments, it is —S(O)₂—O—R′. In some embodiments, it is —S(O)₂—N(R′)₂. In some embodiments, it is —P(O)(-L-R′)₂. In some embodiments, it is —P(O)(R′)₂. In some embodiments, it is —P(O)(OR′)₂. In some embodiments, it is —P(O)[N(R′)₂]₂.

In some embodiments, L^{LG3} is -L^{LG3a}-L^{LG3b}-, wherein L^{LG3a} is a covalent bond or —C(O)O—CH₂—, wherein —CH₂— is optionally substituted, and L^{LG3b} is an optionally substituted aryl ring. In some embodiments, L^{LG3a} is bonded to L^{LG2}, and L^{LG3b} is bonded to L^{LG4}.

In some embodiments, L^{LG3a} is a covalent bond. In some embodiments, L^{LG3a} is —C(O)O—CH₂—, wherein —CH₂— is optionally substituted. In some embodiments, L^{LG3a} is —C(O)O—CH₂—, wherein —CH₂— is substituted. In some embodiments, L^{LG3a} is —C(O)O—CH₂—, wherein —CH₂— is unsubstituted.

In some embodiments, a first group, an antibody binding moiety, and/or LG is released as part of a compound having the structure of R^{LG}-L^{LG1}-L^{LG2}-H or a salt thereof.

In some embodiments, L^{LG3b} is an optionally substituted phenyl ring. In some embodiments, at least one substituent is an electron-withdrawing group as described herein.

In some embodiments, L^{LG3} is

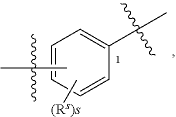

wherein s is 0-4, each R^{s} is independently halogen, —NO₂, -L-R′, —C(O)-L-R′, —S(O)-L-R′, —S(O)₂-L-R′, or —P(O)(-L-R′)₂. In some embodiments, C₁ is bonded to L^{LG4}. In some embodiments, L^{LG3} is

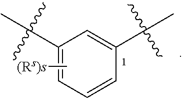

In some embodiments, L^{LG3} is

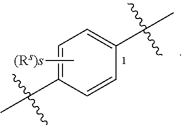

In some embodiments, L^{LG3} is

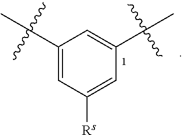

In some embodiments, L^{LG3} is

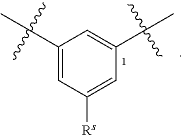

In some embodiments, L^{LG3} is

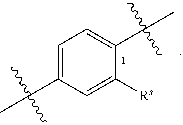

In some embodiments, L^{LG3} is

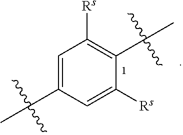

In some embodiments, L^{LG3b} is

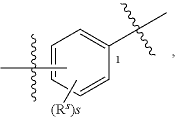

wherein s is 0-4, each R^{s} is independently halogen, —NO₂, -L-R′, —C(O)-L-R′, —S(O)-L-R′, —S(O)₂-L-R′, or —P(O)(-L-R′)₂. In some embodiments, C1 is bonded to L^{LG4}. In some embodiments, L^{LG3b} is

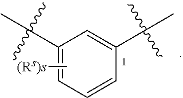

In some embodiments, L^{LG3b} is

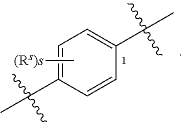

In some embodiments, L^{LG3b} is

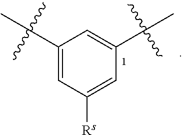

In some embodiments, L^{LG3b} is

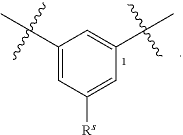

In some embodiments, L^{LG3b} is

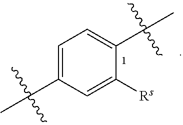

In some embodiments, L^{LG3b} is

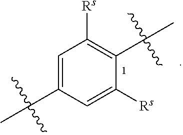

In some embodiments, s is 0. In some embodiments, s is 1-4. In some embodiments, s is 1. In some embodiments, s is 2. In some embodiments, s is 3. In some embodiments, s is 4.

In some embodiments, s is 1-4, and at least one R^{s} is an electron-withdrawing group, e.g., an electron-withdrawing group described above. In some embodiments, at least one R^{s} is —NO₂. In some embodiments, at least one R^{s} is —F. In some embodiments, each R^{s} is independently an electron-withdrawing group. In some embodiments, each R^{s} is —NO₂. In some embodiments, each R^{s} is —F.

In some embodiments, an electron-withdrawing group or R^{s} is at C2. In some embodiments, an electron-withdrawing group or R^{s} is at C₃. In some embodiments, an electron-withdrawing group or R^{s} is at C4. In some embodiments, an electron-withdrawing group or R^{s} is at C2 and C₅.

In some embodiments, L^{LG3} is

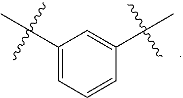 [SMILES: *c1cccc(*)c1]

In some embodiments, L^{LG3} is

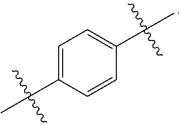 [SMILES: *c1ccc(*)cc1]

In some embodiments, L^{LG3} is

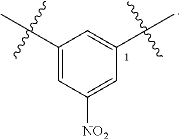 [SMILES: *c1cc(*)cc([N+](=O)[O-])c1]

In some embodiments, L^{LG3} is

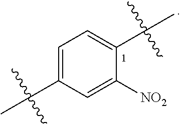 [SMILES: *c1ccc(*)c([N+](=O)[O-])c1]

In some embodiments, L^{LG3} is

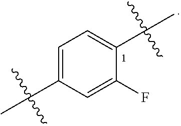 [SMILES: *c1ccc(*)c(F)c1]

In some embodiments, L^{LG3} is

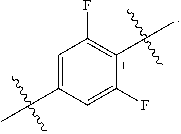 [SMILES: *c1cc(F)c(*)c(F)c1]

In some embodiments, L^{LG3} is

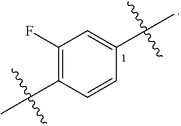 [SMILES: *c1ccc(*)c(F)c1]

In some embodiments, L^{LG3} is

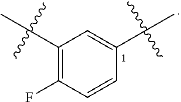 [SMILES: *c1ccc(F)c(*)c1]

In some embodiments, L^{LG3b} is

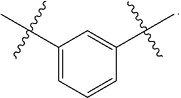 [SMILES: *c1cccc(*)c1]

In some embodiments, L^{LG3b} is

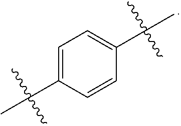 [SMILES: *c1ccc(*)cc1]

In some embodiments, L^{LG3b} is

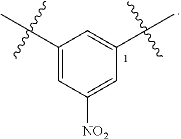 [SMILES: *c1cc(*)cc([N+](=O)[O-])c1]

In some embodiments, L^{LG3b} is

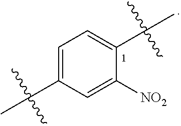 [SMILES: *c1ccc(*)c([N+](=O)[O-])c1]

In some embodiments, L^{LG3b} is

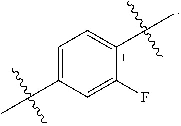 [SMILES: *c1ccc(*)c(F)c1]

In some embodiments, L^{LG3b} is

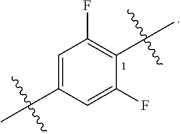 [SMILES: *c1cc(F)c(*)c(F)c1]

In some embodiments, L^{LG3b} is

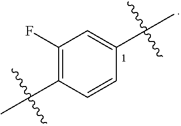 [SMILES: *c1ccc(*)c(F)c1]

In some embodiments, L^{LG3b} is

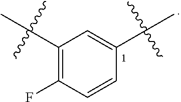 [SMILES: *c1ccc(F)c(*)c1]

In some embodiments, L^{LG3b} is optionally substituted

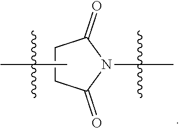

In some embodiments, the nitrogen atom is bound to L^{LG4} which is —O—. In some embodiments, the nitrogen atom is bound to L^{LG4} which is —O—, and -L^{RG1}-L^{RG2}- is —C(O)—.

In some embodiments, -L^{LG4}-L^{RG1}-L^{RG2}- is —O—C(O)—. In some embodiments, -L^{LG4}-L^{RG1}-L^{RG2}- is —S—C(O)—.

In some embodiments, L^{LG4} is a covalent bond. In some embodiments, L^{LG4} is not a covalent bond. In some embodiments, L^{LG4} is —O—. In some embodiments, L^{LG4} is —N(R′)—. In some embodiments, L^{LG4} is —NH—. In some embodiments, L^{LG4} is —N(CH₃)—. In some embodiments, L^{LG4} is —N(R′)—, and L^{LG3} is —O—. In some embodiments, R′ is optionally substituted C₁₋₆ alkyl. In some embodiments, L^{LG4} is —S—.

As described herein, in some embodiments, R^{LG} is or comprises an antibody binding moiety. In some embodiments, R^{LG} is or comprises a protein binding moiety. In some embodiments, R^{LG} is or comprises an antibody binding moiety. In some embodiments, R^{LG} is an antibody binding moiety. In some embodiments, R^{LG} is a protein binding moiety. In some embodiments, R^{LG} is an antibody binding moiety.

In some embodiments, R^{LG} is

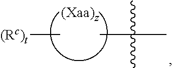

R^{c}-(Xaa)z-, a nucleic acid moiety, or a small molecule moiety. In some embodiments, R^{LG} is or comprises

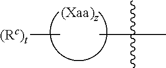

as described herein. In some embodiments, R^{LG} is or comprises R^{c}-(Xaa)z- as described herein. In some embodiments, R^{LG} is or comprises a small molecule moiety. In some embodiments, R^{LG} is or comprises a peptide agent. In some embodiments, R^{LG} is or comprises a nucleic acid agent. In some embodiments, R^{LG} is or comprises an aptamer agent. In some embodiments, an antibody binding moiety is or comprises

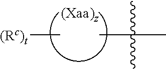

as described herein. In some embodiments, a protein binding moiety is or comprises

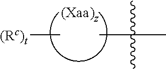

as described herein. In some embodiments, an antibody binding moiety is or comprises

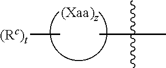

as described herein. In some embodiments, an antibody binding moiety is or comprises R^{c}-(Xaa)z- as described herein. In some embodiments, a protein binding moiety is or comprises R^{c}-(Xaa)z- as described herein. In some embodiments, an antibody binding moiety is or comprises R^{c}-(Xaa)z- as described herein.

In some embodiments, target binding moieties may be conjugated to antibody moieties optionally through linker moieties utilizing technologies described in US 2020/0190165 in accordance with the present disclosure.

In some embodiments, where a particular protecting group (“PG”), leaving group (“LG”), or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

In some embodiments, leaving groups include but are not limited to, halogens (e.g. fluoride, chloride, bromide, iodide), sulfonates (e.g. mesylate, tosylate, benzenesulfonate, brosylate, nosylate, triflate), diazonium, and the like.

In some embodiments, an oxygen protecting group includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

One of skill in the art will appreciate that provided agents may contain one or more stereocenters, and may be present as a racemic or diastereomeric mixture. One of skill in the art will also appreciate that there are many methods known in the art for the separation of isomers to obtain stereoenriched or stereopure isomers of those compounds, including but not limited to HPLC, chiral HPLC, fractional crystallization of diastereomeric salts, kinetic enzymatic resolution (e.g. by fungal-, bacterial-, or animal-derived lipases or esterases), and formation of covalent diastereomeric derivatives using an enantioenriched reagent.

One of skill in the art will appreciate that various functional groups present in compounds of the present disclosure such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. “March's Advanced Organic Chemistry”, 5^{th} Ed., Ed.: Smith, M. B. and March, J., John Wiley & Sons, New York: 2001, the entirety of which is incorporated herein by reference. Such interconversions may require one or more of the aforementioned techniques, and certain methods for synthesizing compounds of the present disclosure are described below in the Exemplification.

As appreciated by those skilled in the art, reaction partners are generally contacted with each other under conditions and for a time sufficient for production of the desired results, e.g., formation of product agents and compositions thereof to desired extents. Many reaction conditions/reaction times may be assessed and utilized if they are suitable for desired purposes in accordance with the present disclosure; certain such conditions, reaction times, assessment, etc. are described in the Examples.

In some embodiments, an agent formed, e.g., a product MATE agent, has the structure of formula M-I or M-II, or a salt thereof. In some embodiments, a target binding moiety in a product agent (e.g., a MATE agent) is the same as a target binding moiety in a reaction partner (e.g., a first agent comprising a target binding moiety) utilized to prepare a product agent. In some embodiments, an antibody moiety in a product agent (e.g., a MATE agent) is the same as an antibody moiety in a reaction partner (e.g., a second agent comprising an antibody moiety) utilized to prepare a product agent.

In some embodiments, linker moieties (or a part thereof) connected to target binding moieties and/or antibody moieties may be transferred from reaction partners (e.g., L^{RM} of formula R-I or a salt thereof). In some embodiments, a linker moiety in a product agent (may be referred to as L^{PM}; e.g., L in formula M-I or M-II) is or comprises a linker moiety in a reaction partner (e.g., one between a reactive group and a moiety of interest, e.g., L^{RM}). In some embodiments, L^{PM} is or comprises L^{RM}. In some embodiments, L^{PM} is -L^{RM}-L^{RG2}-. In some embodiments, L^{RG2} is —C(O)—. In some embodiments, L^{RG2} is —C(O)—, and is bonded to —NH— of a target agent moiety, e.g., —NH— in a side chain of a lysine residue of a protein moiety, which in some embodiments, is an antibody moiety.

Reaction partners, e.g., compounds of formula R-I or salts thereof, typically do not contain moieties that can react with reactive groups under conditions under which reactive groups react with target agents. In some embodiments, to the extent that some moieties in reaction partners may react with reactive groups under conditions under which reactive groups react with target agents, reactions between such moieties and reactive groups are significantly slower and/or less efficient compared to reactions between reactive groups and target agents. In some embodiments, reactions between such moieties and reactive groups do not significantly reduce (e.g., no more than about 5%, 10%, 15%, 20%, 25%, 30%, 40%, 50%, 60%, 70%, 80%, 90%, etc. of reduction) efficiencies, yields, rates, and/or conversions, etc., of reactions between reactive groups and target agents. In some embodiments, reactive groups (e.g., ester groups, activated carboxylic acid derivatives, etc.) react with amino groups (e.g., —NH₂ groups) of target agents (e.g., protein agents such as antibody agents). In some embodiments, reaction partners, e.g., compounds of formula R-I or salts thereof, do not contain amine groups. In some embodiments, compounds of formula R-I or salts thereof (or portions thereof, such as R^{LG}, L^{LG}, L^{LG1}, L^{LG2}, L^{LG3}, L^{LG4}, L^{RG1}, L^{RG2}, L^{RM}, and/or MOI) do not contain amine groups. In some embodiments, they do not contain primary amine groups (—NH₂). In some embodiments, they do not contain —CH₂NH₂. In some embodiments, they do not contain —CH₂CH₂NH₂. In some embodiments, they do not contain —CH₂CH₂CH₂NH₂. In some embodiments, they do not contain —CH₂CH₂CH₂CH₂NH₂. In some embodiments, amine groups, e.g., primary amine groups, are capped (e.g., by introduction of acyl groups (e.g., R—C(O)— (e.g., acetyl)) to form amide groups) to prevent or reduce undesired reactions.

In some embodiments, reactions are performed in buffer systems. In some embodiments, buffer systems of present disclosure maintains structures and/or functions of target agents, moiety of interest, etc. In some embodiments, a buffer is a phosphate buffer. In some embodiments, a buffer is a PBS buffer. In some embodiments, a buffer is a borate buffer. In some embodiments, buffers of the present disclosure provide and optionally maintain certain pH value or range. For example, in some embodiments, a useful pH is about 7-9, e.g., 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 9.0, etc. In some embodiments, a pH is 7.4. In some embodiments, a pH is 7.5. In some embodiments, a pH is 7.8. In some embodiments, a pH is 8.0. In some embodiments, a pH is 8.2. In some embodiments, a pH is 8.3.

Provided technologies can provide various advantages. Among other things, in some embodiments, connection of a moiety of interest in a reaction partner (e.g., a compound comprising a reactive group located between an antibody binding moiety and a moiety of interest (e.g., a compound of formula R-I or a salt thereof)) to an agent comprising an antibody moiety (e.g., a second agent such as an antibody agent) and release of an antibody binding moiety in a provided reaction partner can be achieved in one reaction and/or in one pot. Thus, in many embodiments, no separate reactions/steps are performed to remove antibody binding moieties. As appreciated by those skilled in the art, by performing connection of moiety of interest and release of antibody binding moiety in a single reaction/operation, provided technologies can avoid separate steps for antibody binding moiety removal and can improve overall efficiency (e.g., by simplify operations, increasing overall yield, etc.), reduce manufacturing cost, improve product purity (e.g., by avoiding exposure to antibody binding moiety removal conditions, which typically involve one or more of reduction, oxidation, hydrolysis (e.g., of ester groups), etc., conditions and may damage target agent moieties (e.g., for protein agent moieties, protein amino acid residues, overall structures, and/or post-translational modifications (e.g., glycans of antibodies) thereof. In some embodiments, provided technologies among other things can provided improved efficiency (e.g., in terms of reaction rates and/or conversion percentages), increased yield, increased purity/homogeneity, and/or enhanced selectivity, particularly compared to reference technologies wherein a reaction partner containing no antibody binding moieties is used, without introducing step(s) for antibody binding moiety removal (e.g., antibody binding moiety is removed in the same step as moiety of interest conjugation).

In some embodiments, the present disclosure provides products of provided processes, which, among other things, contain low levels of damage to antibody moieties compared to processes comprising steps which are performed for antibody binding moiety removal but not for substantial conjugation of moieties of interest (e.g. target binding moieties). In some embodiments, provided product agent compositions have high homogeneity (e.g., with respect to the number of moiety of interest per antibody moiety, and/or positions of amino acid residues in antibody moieties conjugated to moieties of interest) compared to reference product compositions (e.g., those from technologies without using antibody binding moieties, or utilizing extra step(s) for antibody binding moiety removal (e.g., not utilizing reaction partners described herein which comprise a reactive group located between an antibody binding moiety and a moiety of interest).

In some embodiments, the present disclosure provides a product agent which is an agent comprising an antibody moiety, a target binding moiety and optionally a linker moiety linking an antibody binding moiety and a target binding moiety. In some embodiments, the present disclosure provides compositions of such agents.

In some embodiments, the present disclosure provides a composition comprising a plurality of agents, wherein each agent independently comprises:
an antibody moiety,
a target binding moiety, and
optionally a linker moiety linking an antibody binding moiety and a target binding moiety.

In some embodiments, product agents are MATE agents. In some embodiments, an antibody agent moiety comprises IgG Fc region. In some embodiments, an antibody moiety is connected to a moiety of interest through an amino group optionally through a linker. In some embodiments, it is through a lysine residue wherein the amino group of the side chain is connected to a moiety of interest optionally through a linker (e.g., forming —NH—C(O)— as part of an amide group, a carbamate group, etc.).

In some embodiments, selected locations of antibody moieties are utilized for conjugation. For example, in some embodiments, K246 or K248 of an antibody agent (EU numbering, or corresponding residues) are conjugation locations. In some embodiments, a conjugation location is K246 of heavy chain (unless otherwise specified, locations herein include corresponding residues in, e.g., modified sequence (e.g., longer, shorter, rearranged, etc., sequences)). In some embodiments, a location is K248 of heavy chain. In some embodiments, a location is K288 or K290 of heavy chain. In some embodiments, a location is K288 of heavy chain. In some embodiments, a location is K290 of heavy chain. In some embodiments, a location is K317. In some embodiments, an antibody moiety is a moiety of an IgG1 antibody or a fragment thereof. In some embodiments, an antibody moiety is a moiety of an IgG2 antibody or a fragment thereof. In some embodiments, an antibody moiety is a moiety of an IgG4 antibody or a fragment thereof. In some embodiments, a composition comprises a plurality of MATE agents, wherein antibody moieties of the plurality of MATE agents are independently an antibody moiety of an IgG1, IgG2, or IgG4 antibody, or a fragment thereof.

In some embodiments, antibody heavy chains are selectively conjugated/labeled over light chains.

Among other things, the present disclosure can provide controlled moiety of interest (e.g., a target binding moiety)/antibody moiety ratios (e.g., for moiety of interest being peptide target binding moiety, peptide target binding moiety/antibody ratio (PAR)). In some embodiments, a ratio is about 0.1-10, 0.5-6, etc., e.g., about 0.1, 0.2, 0.3, 0.4, 0.5, 0.6, 0.7, 0.8, or 0.9 to about 1, 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 4.5, 5.0, 5.5, 6, 7, 8, 9, 10, about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 4.5, 5.0, 5.5, 6, 7, 8, 9, 10, etc.). In some embodiments, a ratio is of moieties of interest conjugated to antibody moiety and antibody moieties conjugated to moieties of interest (e.g., when a ratio is in the context of a ratio of an agent). In some embodiments, a ratio is of moieties of interest conjugated to antibody moieties and all antibodies in a composition (e.g., when a ratio is in the context of a ratio of a composition). In some embodiments, a ratio is about 0.1-6. In some embodiments, a ratio is about 0.5-2.5. In some embodiments, a ratio is about 0.5-2. In some embodiments, a ratio is about 1-2. In some embodiments, a ratio is about 1.5-2. In some embodiments, a ratio is about 1.5-2 for IgG1, IgG2 and/or IgG4 antibodies or fragments thereof. In some embodiments, for a composition, e.g., target binding moieties conjugated to IVIG, a ratio is about 1.5-2.5. In some embodiments, a ratio is about 0.1. In some embodiments, a ratio is about 0.2. In some embodiments, a ratio is about 0.3. In some embodiments, a ratio is about 0.4. In some embodiments, a ratio is about 0.5. In some embodiments, a ratio is about 0.6. In some embodiments, a ratio is about 0.7. In some embodiments, a ratio is about 0.8. In some embodiments, a ratio is about 0.9. In some embodiments, a ratio is about 1. In some embodiments, a ratio is about 1.1. In some embodiments, a ratio is about 1.2. In some embodiments, a ratio is about 1.3. In some embodiments, a ratio is about 1.4. In some embodiments, a ratio is about 1.5. In some embodiments, a ratio is about 1.6. In some embodiments, a ratio is about 1.7. In some embodiments, a ratio is about 1.8. In some embodiments, a ratio is about 1.9. In some embodiments, a ratio is about 2. In some embodiments, a ratio is about 2.1. In some embodiments, a ratio is about 2.2. In some embodiments, a ratio is about 2.3. In some embodiments, a ratio is about 2.4. In some embodiments, a ratio is about 2.5. In some embodiments, a ratio is about 1.8 for a composition wherein antibody moieties of a plurality of agents are those of an IVIG preparation. The term IVIG preparation can also include similar human Ig preparation for other modes of administration such as subcutaneous (IGSC) or IM injection. For example the immunoglobin can be subcutaneous Ig (IGSC, such as the human subcutaneous immunoglobin products, including the brands CUTAQUIG (16.5% Ig solution, from Octapharma, Lachen, Switzerland), HIZENTRA (human subcutaneous immunoglobin, 20% Ig solution from CSL Bering, King of Prussia, NJ, USA), or XEMBIFY (human subcutaneous immunoglobin, 20% Ig solution from Grifols, Barcelona, Spain) or intravenous immunoglobin (IVIG, such as human intravenous immunoglobin products, including the brands BIVIGAM (human intravenous Ig, 10% solution, from ADMA Biologics, Ramsey NJ, USA) and GAMUNEX-C(human injectable Ig, 10% solution, from Grifols, Barcelona, Spain).

In some embodiments, a ratio of target binding moieties and antibody moieties is about 1.5-2 wherein antibody moieties of a plurality of agents are those of IgG1. In some embodiments, a ratio of target binding moieties and antibody moieties is about 1.5-2 wherein antibody moieties of a plurality of agents are those of IgG2. In some embodiments, a ratio of target binding moieties and antibody moieties is about 1.5-2 wherein antibody moieties of a plurality of agents are those of IgG4. In some embodiments, a ratio is about 1.9-2.

In some embodiments, in provided agents (e.g., agents of formula M-I or M-II, or a salt thereof) substantially all conjugation sites of antibody moieties have the same modifications (e.g., all share the same moieties of interest optionally connected through the same linker moieties). In some embodiments, no conjugation sites bear different modifications (e.g., different moieties of interest and/or no moieties of interest and/or different linker moieties).

In some embodiments, about 10%-100% of all, or substantially all, moieties of interest, e.g., target binding moieties, conjugated to antibody moieties of a particular type of antibodies (e.g., IgG1) or fragments thereof are conjugated to one or more particularly sites, typically one or two particularly sites (e.g., K246 and K248 of an IgG1 heavy chain and amino acid residues corresponding thereto). In some embodiments, about 10%-100% of all, or substantially all, moieties of interest, e.g., target binding moieties, conjugated to antibody moieties of IgG2 antibodies or fragments thereof are at K251 and K253 of an IgG2 heavy chain and amino acid residues corresponding thereto. In some embodiments, about 10%-100% of all, or substantially all, moieties of interest, e.g., target binding moieties, conjugated to antibody moieties of IgG2 antibodies or fragments thereof are at K239 and K241 of an IgG4 heavy chain and amino acid residues corresponding thereto. In some embodiments, about 10%-100% of all, or substantially all, moieties of interest (e.g., for a plurality of agents, for a composition, etc.) are conjugated to antibody moieties of IgG1, IgG2, and/or IgG4 antibodies, or fragments thereof (e.g., for conjugation products with IgG1 antibodies or fragments thereof (antibody moieties being of IgG1 antibodies or fragments thereof), IgG2 antibodies or fragments thereof (antibody moieties being of IgG2 antibodies or fragments thereof), IgG4 antibodies or fragments thereof (antibody moieties being of IgG4 antibodies or fragments thereof), or for conjugation products with IVIG (when certain provided technologies described herein are utilized, selective conjugation with IgG1, IgG2 and IgG4). In some embodiments, a percentage is about 10% or more. In some embodiments, a percentage is about 20% or more. In some embodiments, a percentage is about 25% or more. In some embodiments, a percentage is about 30% or more. In some embodiments, a percentage is about 40% or more. In some embodiments, a percentage is about 50% or more. In some embodiments, a percentage is about 60% or more. In some embodiments, a percentage is about 65% or more. In some embodiments, a percentage is about 70% or more. In some embodiments, a percentage is about 75% or more. In some embodiments, a percentage is about 80% or more. In some embodiments, a percentage is about 85% or more. In some embodiments, a percentage is about 90% or more. In some embodiments, a percentage is about 95% or more. In some embodiments, a percentage is about 100%.

In some embodiments, a composition comprises a plurality of agents (e.g., MATE agents, agents of formula M-I or M-II, or a salt thereof), each independent comprising a target binding moiety, an antibody moiety, and optionally a linker moiety linking a target binding moiety and an antibody moiety. In some embodiments, substantially all target binding moieties of a plurality of agents are the same. In some embodiments, substantially all target binding moieties of a plurality of agents comprise peptide moieties of a common amino acid sequence. In some embodiments, substantially all target binding moieties of a plurality of agents are peptide moieties of a common amino acid sequence. In some embodiments, substantially all conjugation sites of antibody moieties in a plurality of agents have the same modifications (e.g., all share the same moieties of interest optionally connected through the same linker moieties). In some embodiments, no conjugation sites of a plurality of agents bear different modifications (e.g., different moieties of interest and/or no moieties of interest and/or different linker moieties). In some embodiments, a plurality of agents do not contain agents that share the same (or substantially the same) antibody moieties but different modifications (e.g., different moieties of interest and/or no moieties of interest and/or different linker moieties). In some embodiments, agents that share the same (or substantially the same) antibody moieties but different modifications (e.g., different moieties of interest and/or no moieties of interest and/or different linker moieties) are intermediates of multiple-step preparations (e.g., comprising steps for removal of antibody binding moieties in addition to steps for moiety of interest conjugation) of final product agents.

In some embodiments, the present disclosure provides a composition comprising a plurality of agents each of which independently comprising:
an antibody moiety,
a moiety of interest, and
optionally a linker moiety linking the antibody moiety and the moiety of interest;
   wherein antibody moieties of agents of the plurality comprise a common amino acid sequence, and agents of the plurality share a common moiety of interest independently at at least one common amino acid residue of the common amino acid sequence; and
wherein about 1%-100% of all agents that comprise an antibody moiety that comprise the common amino acid sequence and the moiety of interest are agents of the plurality.

In some embodiments, the present disclosure provides a composition comprising a plurality of agents each of which independently comprising:
an antibody moiety,
a moiety of interest, and
optionally a linker moiety linking an antibody moiety and a moiety of interest;
   wherein agents of the plurality share the same or substantially the same antibody moiety, and a moiety of interest at at least one common location; and
wherein about 1%-100% of all agents that comprise the antibody moiety and the moiety of interest are agents of the plurality.

In some embodiments, an antibody moiety is a moiety of an IgG1 antibody or a fragment thereof. In some embodiments, an antibody moiety is a moiety of an IgG2 antibody or a fragment thereof. In some embodiments, an antibody moiety is a moiety of an IgG3 antibody or a fragment thereof. In some embodiments, an antibody moiety is a moiety of an IgG4 antibody or a fragment thereof. In some embodiments, about 1-100% of all moieties of interest are at common location(s). In some embodiments, a moiety of interest is a target binding moiety as described herein. In some embodiments, agents of a plurality are each independently of formula M-I or M-II, or a salt thereof.

In some embodiments, antibody moieties of agents of a plurality comprise a common amino acid sequence. In some embodiments, antibody moieties of agents of a plurality comprise a common amino acid sequence in a Fc region. In some embodiments, antibody moieties of agents of a plurality comprise a common Fc region. In some embodiments, antibody moieties of agents of a plurality can bind a common antigen specifically. In some embodiments, antibody moieties are monoclonal antibody moieties. In some embodiments, antibody moieties are polyclonal antibody moieties. In some embodiments, antibody moieties bind to two or more different antigens. In some embodiments, antibody moieties bind to two or more different proteins. In some embodiments, antibody moieties are IVIG moieties.

In some embodiments, a moiety of interest in an agent of a plurality is a target binding moiety. In some embodiments, each moiety of interest is independently a target binding moiety. In some embodiments, a composition comprises a plurality of agents, antibody moieties of agents of the plurality comprise a common amino acid sequence, and agents of a plurality share a common target binding moiety independently linked to a common amino acid residue in the common amino acid sequence, each independently and optionally through a linker; and wherein about 1%-100% of all agents that comprise an antibody moiety that comprises a common amino acid sequence and a common target binding moiety independently comprise a common target binding moiety linked to a common amino acid residue independently and optionally through a linker. In some embodiments, a composition comprises a plurality of agents, antibody moieties of agents of a plurality comprise a common amino acid sequence, and agents of a plurality share a common target binding moiety independently linked to a common amino acid residue in the common amino acid sequence, each independently through a common linker; and wherein about 1%-100% of all agents that comprise an antibody moiety that comprises a common amino acid sequence and a common target binding moiety independently comprise a common target binding moiety linked to a common amino acid residue independently and through a common linker. In some embodiments, a composition comprises a plurality of agents, antibody moieties of agents of a plurality comprise a common amino acid sequence, and agents of a plurality share a common target binding moiety independently linked to a common amino acid residue in the common amino acid sequence, each independently and optionally through a linker; and wherein about 1%-100% of all agents that comprise an antibody moiety that comprises a common amino acid sequence and a common target binding moiety are agents of a plurality. In some embodiments, a composition comprises a plurality of agents, wherein antibody moieties of agents of a plurality comprise a common amino acid sequence, and agents of a plurality share a common target binding moiety independently linked to a common amino acid residue in the common amino acid sequence, each independently through a common linker; and wherein about 1%-100% of all agents that comprise an antibody moiety that comprises a common amino acid sequence, a common target binding moiety, and a common linker are agents of a plurality.

As used in the present disclosure, in some embodiments, “at least one” or “one or more” is 1-1000, 1-500, 1-200, 1-100, 1-90, 1-80, 1-70, 1-60, 1-50, 1-40, 1-30, 1-20, 1-10, 1-5, or about 1, 2, 3, 4, 5, 6, 7, 8, 9, 10, 11, 12, 13, 14, 15, 16, 17, 18, 19, 20, or more. In some embodiments, it is one. In some embodiments, it is two or more. In some embodiments, it is about 3. In some embodiments, it is about 4. In some embodiments, it is about 5. In some embodiments, it is about 6. In some embodiments, it is about 7. In some embodiments, it is about 8. In some embodiments, it is about 9. In some embodiments, it is about 10. In some embodiments, it is about 10 or more.

In some embodiments, a common amino acid sequence comprises 1-1000, 1-500, 1-400, 1-300, 1-200, 1-100, 1-50, 10-1000, 10-500, 10-400, 10-300, 10-200, 10-100, 10-50, 20-1000, 20-500, 20-400, 20-300, 20-200, 20-100, 20-50, 50-1000, 50-500, 50-400, 50-300, 50-200, 50-100, 1, 2, 3, 4, 5, 6, 7, 8, 9, 10, 11, 12, 13, 14, 15, 16, 17, 18, 19, 20, 25, 30, 40, 50, 60, 70, 80, 90, 100, 110, 120, 130, 140, 150, 200, 250, 300, 400, 500, 600 or more amino acid residues. In some embodiments, a length is at least 5 amino acid residues. In some embodiments, a length is at least 10 amino acid residues. In some embodiments, a length is at least 50 amino acid residues. In some embodiments, a length is at least 100 amino acid residues. In some embodiments, a length is at least 150 amino acid residues. In some embodiments, a length is at least 200 amino acid residues. In some embodiments, a length is at least 300 amino acid residues. In some embodiments, a length is at least 400 amino acid residues. In some embodiments, a length is at least 500 amino acid residues. In some embodiments, a length is at least 600 amino acid residues.

In some embodiments, a common amino acid sequence is at least 10%-100%, 50%-100%, 50%, 55%, 60%, 65%, 70%, 75%, 80%, 85%, 90%, 91%, 92%, 93%, 94%, 95%, 96%, 97%, 98% or 99% of an amino acid sequence of an antibody moiety, a protein agent moiety, etc. In some embodiments, it is 10% or more. In some embodiments, it is 20% or more. In some embodiments, it is 30% or more. In some embodiments, it is 40% or more. In some embodiments, it is 50% or more. In some embodiments, it is 60% or more. In some embodiments, it is 70% or more. In some embodiments, it is 80% or more. In some embodiments, it is 90% or more. In some embodiments, it is 100%.

In some embodiments, in a common amino acid sequence, one and only one amino acid residue is linked to a common moiety of interest, e.g., a common target binding moiety. In some embodiments, in a common amino acid sequence, two and only two amino acid residues are linked to a common moiety of interest, e.g., a common target binding moiety. In some embodiments, in a common amino acid sequence, two or more amino acid residues are linked to a common moiety of interest, e.g., a common target binding moiety. In some embodiments, each common moiety of interest, e.g., a common target binding moiety, is independently linked to an amino acid residue in a common amino acid sequence.

In some embodiments, a common amino acid sequence comprises one or more amino acid residues selected from K246 and K248 of an IgG1 heavy chain and amino acid residues corresponding thereto, K251 and K253 of an IgG2 heavy chain and amino acid residues corresponding thereto, and K239 and K241 of an IgG4 heavy chain and amino acid residues corresponding thereto. In some embodiments, a common amino acid sequence comprises one or more amino acid residues selected from K246 and K248 of an IgG1 heavy chain and amino acid residues corresponding thereto. In some embodiments, a common amino acid sequence comprises one or more amino acid residues selected from K251 and K253 of an IgG2 heavy chain and amino acid residues corresponding thereto. In some embodiments, a common amino acid sequence comprises one or more amino acid residues selected from K239 and K241 of an IgG4 heavy chain and amino acid residues corresponding thereto. In some embodiments, a moiety of interest is connected to such an amino acid residue (unless explicitly noted, optionally through a linker moiety). In some embodiments, each moiety of interest is connected to such an amino acid residue each optionally and independently through a linker moiety.

In some embodiments, antibody moieties share a high percentage of amino acid sequence homology. In some embodiments, it is about 50%-100%. In some embodiments, it is 50%. In some embodiments, it is 60%. In some embodiments, it is 70%. In some embodiments, it is 80%. In some embodiments, it is 90%. In some embodiments, it is 91%. In some embodiments, it is 50%. In some embodiments, it is 92%. In some embodiments, it is 93%. In some embodiments, it is 94%. In some embodiments, it is 95%. In some embodiments, it is 96%. In some embodiments, it is 97%. In some embodiments, it is 98%. In some embodiments, it is 99%. In some embodiments, it is 100%. In some embodiments, it is at least 50%. In some embodiments, it is at least 60%. In some embodiments, it is at least 70%. In some embodiments, it is at least 80%. In some embodiments, it is at least 90%. In some embodiments, it is at least 91%. In some embodiments, it is at least 50%. In some embodiments, it is at least 92%. In some embodiments, it is at least 93%. In some embodiments, it is at least 94%. In some embodiments, it is at least 95%. In some embodiments, it is at least 96%. In some embodiments, it is at least 97%. In some embodiments, it is at least 98%. In some embodiments, it is at least 99%.

In some embodiments, a percentage used herein, e.g., about 1%-100%, is about 10% or more. In some embodiments, a percentage is about 20% or more. In some embodiments, a percentage is about 25% or more. In some embodiments, a percentage is about 30% or more. In some embodiments, a percentage is about 40% or more. In some embodiments, a percentage is about 50% or more. In some embodiments, a percentage is about 60% or more. In some embodiments, a percentage is about 65% or more. In some embodiments, a percentage is about 70% or more. In some embodiments, a percentage is about 75% or more. In some embodiments, a percentage is about 80% or more. In some embodiments, a percentage is about 85% or more. In some embodiments, a percentage is about 90% or more. In some embodiments, a percentage is about 95% or more. In some embodiments, a percentage is about 100%.

In some embodiments, antibody moiety of agents of a plurality comprise a common Fc region or a fragment thereof.

In some embodiments, moieties of interest of agents of a plurality are at particular locations. In some embodiments, all moieties of interest are at amino acid residues of a common amino acid sequence. In some embodiments, all moieties of interest are at common locations of amino acid residues of a common amino acid sequence. In some embodiments, the number of common locations is 1. In some embodiments, it is 2. In some embodiments, it is 3. In some embodiments, it is 4. In some embodiments, antibody moieties comprise two heavy chains or fragments thereof, and the number of common locations is 2 (one on each chain). In some embodiments, common locations are selected from K246 and K248 of an IgG1 heavy chain and amino acid residues corresponding thereto, K251 and K253 of an IgG2 heavy chain and amino acid residues corresponding thereto, and K239 and K241 of an IgG4 heavy chain and amino acid residues corresponding thereto.

In some embodiments, agents of a plurality share a common moiety of interest independently at at least one location. In some embodiments, agents of a plurality share a common moiety of interest and linker independently at at least one location. In some embodiments, moieties of interest at two or more or all locations comprise a common moiety of interest. In some embodiments, moieties of interest are the same.

In some embodiments, agents share a common modification at least one common amino acid residue. In some embodiments, agents of a plurality share a common modification at each location which is connected to a moiety of interest and optionally a linker. In some embodiments, agents of a plurality the same -L^{PM}-MOI at each location that is connected to a linker moiety.

In some embodiments, a location is selected from K246, K248, K288, K290, K317 of antibody agents and locations corresponding thereto. In some embodiments, a location is selected from K246 and K248, and locations corresponding thereto. In some embodiments, a location is selected from K288 and K290, and locations corresponding thereto. In some embodiments, a location is K246 or a location corresponding thereto. In some embodiments, a location is K248 or a location corresponding thereto. In some embodiments, a location is K288 or a location corresponding thereto. In some embodiments, a location is K290 or a location corresponding thereto. In some embodiments, a location is K317 or a location corresponding thereto. In some embodiments, a location is K185 of light chain or a location corresponding thereto. In some embodiments, a location is K187 of light chain or a location corresponding thereto. In some embodiments, a location is K133 of heavy chain or a location corresponding thereto. In some embodiments, a location is K246 or K248 of heavy chain or a location corresponding thereto. In some embodiments, a location is K414 of heavy chain or a location corresponding thereto. In some embodiments, a common sequence is a sequence that is about or at least about 10-100, 20-50, e.g., about or at least about 10, 20, 30, 40, 50, 60, 70, 80, 90, or 100, amino acid residues in length, and comprises one or more of such residues or residues corresponding thereto. In some embodiments, a common sequence is a sequence that is about or at least about 10-100, 20-50, e.g., about or at least about 10, 20, 30, 40, 50, 60, 70, 80, 90, or 100, amino acid residues in length, and comprises one, two or more residues selected from K246 and K248 of an IgG1 heavy chain and amino acid residues corresponding thereto, K251 and K253 of an IgG2 heavy chain and amino acid residues corresponding thereto, and K239 and K241 of an IgG4 heavy chain and amino acid residues corresponding thereto.

In some embodiments, about 1%-100% of all agents that comprise an antibody moiety and a moiety of interest are agents of a plurality. In some embodiments, about 1%-100% of all agents that comprise an antibody moiety that comprises a common amino acid sequence and a moiety of interest are agents of a plurality. In some embodiments, about 1%-100% of all agents that comprise an antibody moiety that comprise a common amino acid sequence or can bind to a common antigen and a moiety of interest are agents of a plurality. In some embodiments, about 1%-100% of all agents that comprise an antibody moiety are agents of a plurality. In some embodiments, about 1%-100% of all agents that comprise an antibody moiety that comprise the common amino acid sequence are agents of a plurality. In some embodiments, about 1%-100% of all agents that comprise a protein agent moiety that comprise the common amino acid sequence are agents of a plurality. In some embodiments, about 1%-100% of all agents that comprise an antibody agent moiety that comprise the common amino acid sequence or can bind to the common antigen are agents of a plurality. In some embodiments, a percentage is about 5%-100%. In some embodiments, a percentage is about 10%-100%. In some embodiments, a percentage is about 20%-100%. In some embodiments, a percentage is about 25%-100%. In some embodiments, a percentage is about 30%-100%. In some embodiments, a percentage is about 40%-100%. In some embodiments, a percentage is about 50%-100%. In some embodiments, it is about 5%. In some embodiments, it is about 10%. In some embodiments, it is about 20%. In some embodiments, it is about 25%. In some embodiments, it is about 30%. In some embodiments, it is about 40%. In some embodiments, it is about 50%. In some embodiments, it is about 60%. In some embodiments, it is about 70%. In some embodiments, it is about 80%. In some embodiments, it is about 90%. In some embodiments, it is about 91%. In some embodiments, it is about 50%. In some embodiments, it is about 92%. In some embodiments, it is about 93%. In some embodiments, it is about 94%. In some embodiments, it is about 95%. In some embodiments, it is about 96%. In some embodiments, it is about 97%. In some embodiments, it is about 98%. In some embodiments, it is about 99%. In some embodiments, it is about 100%. In some embodiments, it is at least about 5%. In some embodiments, it is at least about 10%. In some embodiments, it is at least about 20%. In some embodiments, it is at least about 25%. In some embodiments, it is at least about 30%. In some embodiments, it is at least about 40%. In some embodiments, it is at least about 50%. In some embodiments, it is at least about 60%. In some embodiments, it is at least about 70%. In some embodiments, it is at least about 80%. In some embodiments, it is at least about 90%. In some embodiments, it is at least about 91%. In some embodiments, it is at least about 50%. In some embodiments, it is at least about 92%. In some embodiments, it is at least about 93%. In some embodiments, it is at least about 94%. In some embodiments, it is at least about 95%. In some embodiments, it is at least about 96%. In some embodiments, it is at least about 97%. In some embodiments, it is at least about 98%. In some embodiments, it is at least about 99%.

In some embodiments, each agent of the plurality does not contain —S-Cy-, wherein -Cy- is optionally substituted 5-membered monocyclic ring, does not contain —S—S— which is not formed by cysteine residues and does not contain —SH or salt form thereof that is not of a cysteine residue. In some embodiments, each agent of the plurality does not contain —S—CH₂—CH₂—. In some embodiments, each agent of the plurality does not contain a moiety that can specifically bind to an antibody agent. In some embodiments, a composition is substantially free from a moiety that can specifically bind to an antibody agent.

In some embodiments, provided agents, compounds, etc., e.g., those of formula R-I, M-I, M-II, etc. and salts thereof have high purity. In some embodiments, a percentage is about 5%-100%. In some embodiments, a percentage is about 10%-100%. In some embodiments, a percentage is about 20%-100%. In some embodiments, a percentage is about 25%-100%. In some embodiments, a percentage is about 30%-100%. In some embodiments, a percentage is about 40%-100%. In some embodiments, a percentage is about 50%-100%. In some embodiments, it is about 5%. In some embodiments, it is about 10%. In some embodiments, it is about 20%. In some embodiments, it is about 25%. In some embodiments, it is about 30%. In some embodiments, it is about 40%. In some embodiments, it is about 50%. In some embodiments, it is about 60%. In some embodiments, it is about 70%. In some embodiments, it is about 80%. In some embodiments, it is about 90%. In some embodiments, it is about 91%. In some embodiments, it is about 50%. In some embodiments, it is about 92%. In some embodiments, it is about 93%. In some embodiments, it is about 94%. In some embodiments, it is about 95%. In some embodiments, it is about 96%. In some embodiments, it is about 97%. In some embodiments, it is about 98%. In some embodiments, it is about 99%. In some embodiments, it is about 100%. In some embodiments, it is at least about 5%. In some embodiments, it is at least about 10%. In some embodiments, it is at least about 20%. In some embodiments, it is at least about 25%. In some embodiments, it is at least about 30%. In some embodiments, it is at least about 40%. In some embodiments, it is at least about 50%. In some embodiments, it is at least about 60%. In some embodiments, it is at least about 70%. In some embodiments, it is at least about 80%. In some embodiments, it is at least about 90%. In some embodiments, it is at least about 91%. In some embodiments, it is at least about 50%. In some embodiments, it is at least about 92%. In some embodiments, it is at least about 93%. In some embodiments, it is at least about 94%. In some embodiments, it is at least about 95%. In some embodiments, it is at least about 96%. In some embodiments, it is at least about 97%. In some embodiments, it is at least about 98%. In some embodiments, it is at least about 99%.

In some embodiments, the present disclosure provides product agent compositions comprising product agents (e.g., agents of formula M-I or M-II, or a salt thereof). In some embodiments, a product agent composition (e.g., a formed agent composition from certain methods) comprises a product agent comprising an antibody moiety and a moiety of interest and optionally a linker (e.g., an agent of formula M-I or M-II, or a salt thereof), a released antibody binding moiety (e.g., a compound comprising R^{LG}-(L^{LG1})₀₋₁-(L^{LG2})₀₋₁-(L^{LG3})₀₋₁-(L^{LG4})₀₋₁-) or a compound comprising a released antibody binding moiety (e.g., a compound having the structure of R^{LG}-(L^{LG1})₀₋₁-(L^{LG2})₀₋₁-(L^{LG3})₀₋₁-(L^{LG4})₀₋₁-H or a salt thereof), and a reaction partner (e.g., a compound of formula R-I or a salt thereof). In some embodiments, released antibody binding moieties may bind to antibody moieties in target agents and/or formed product agents. Various technologies are available to separate released antibody binding moieties from antibody moieties in accordance with the present disclosure, for example, in some embodiments, contacting a composition with a composition comprising glycine at certain pH. In some embodiments, each agent of a plurality is independently such a product agent.

### Reactive Group

In some embodiments, provided agents, compounds, e.g., those useful as reaction partners such as first agents, comprise reactive groups (e.g., RG). In some embodiments, reactive groups (e.g., RG) are located between antibody binding moieties (e.g., ABT) and moieties of interest (e.g., MOI), and are optionally and independently linked to antibody binding moieties and moieties of interest via linkers. In some embodiments, RG is a reaction group as described herein.

In some embodiments, reactive groups when utilized in agents that comprise no antibody binding moieties react slowly and provide low level of, in some embodiments, substantially no conjugation of moieties of interest with target agents. As demonstrated herein, combination of reactive groups with antibody binding moieties in the same agents, e.g., as in compounds of formula R-I or salts thereof, can, among other things, promote reactions between reactive groups and target agents, enhance reaction efficiency, reduce side reactions, and/or improve reaction selectivity (e.g., in terms of target sites wherein conjugation of moieties of interest with target agents occurs).

Reactive groups in agents can react with various types of groups in target agents. In some embodiments, reactive groups in agents selectively react with amino groups of target agents, e.g., —NH₂ groups on side chains of lysine residues of proteins. In some embodiments, reactive groups when utilized in agents, e.g., those of formula R-I or salts thereof, selectively react with particular sites of target agents, e.g., as shown in examples herein, one or more of K246, K248, K288, K290, K317, etc. of IgG1, K251, K 253, etc. for IgG2, K239, K241 for IgG4, etc. In some embodiments, a site is K246 or K248 of an antibody heavy chain. In some embodiments, sites are K246 and/or K248 of an antibody heavy chain. In some embodiments, a site is K246 of an antibody heavy chain. In some embodiments, a site is K248 of an antibody heavy chain. In some embodiments, a site is K288 or K290 of an antibody heavy chain. In some embodiments, a site is K288 of an antibody heavy chain. In some embodiments, a site is K290 of an antibody heavy chain. In some embodiments, a site is K317. In some embodiments, a site is K414 of an antibody heavy chain. In some embodiments, a site is K185 of an antibody light chain. In some embodiments, a site is K187 of an antibody light chain. In some embodiments, sites are K251 and/or K253 of an IgG2 heavy chain. In some embodiments, a site is K251 of an IgG2 heavy chain. In some embodiments, a site is K253 of an IgG2 heavy chain. In some embodiments, sites are K239 and/or K241 of an IgG4 heavy chain. In some embodiments, a site is K239 of an IgG4 heavy chain. In some embodiments, a site is K241 of an IgG4 heavy chain. In some embodiments, conjugation selectively occurs at one or more heavy chain sites over light chain sites. In some embodiments, for technologies without antibody binding moieties, conjugation occurs at light chain sites more than heavy chain sites.

In some embodiments, a reactive group, e.g., RG, is or comprises an ester group. In some embodiments, a reactive group, e.g., RG, is or comprises an electrophilic group, e.g., a Michael acceptor.

In some embodiments, a reactive group, e.g., RG, is or comprises -L^{RG1}-L^{RG2}-, wherein each of L^{RG1} and L^{RG2} is independently L as described herein. In some embodiments, a reactive group, e.g., RG, is or comprises -L^{LG4}-L^{RG1}-L^{RG2}-, wherein each variable is as described herein. In some embodiments, a reactive group, e.g., RG, is or comprises -L^{LG3}-L^{LG4}-L^{RG1}-L^{RG2}-, wherein each variable is as described herein. In some embodiments, a reactive group, e.g., RG, is or comprises -L^{LG2}-L^{LG3}-L^{LG4}-L^{RG1}-L^{RG2}-, wherein each variable is as described herein. In some embodiments, a reactive group, e.g., RG, is or comprises -L^{LG4}-L^{RG2}-, wherein each variable is as described herein. In some embodiments, a reactive group, e.g., RG, is or comprises -L^{LG3}-L^{LG4}-L^{RG2}-, wherein each variable is as described herein. In some embodiments, a reactive group, e.g., RG, is or comprises -L^{LG2}-L^{LG3}-L^{LG4}-L^{RG2}-, wherein each variable is as described herein.

In some embodiments, as described herein, L^{LG4} is —O—. In some embodiments, L^{LG4} is —N(R)—. In some embodiments, L^{LG4} is —NH—.

In some embodiments, as described herein, L^{LG3} is or comprises an optionally substituted aryl ring. In some embodiments, L^{LG3} is or comprises a phenyl ring. In some embodiments, an aryl or phenyl ring is substituted. In some embodiments, a substituent is an electron-withdrawing group as described herein, e.g., —NO₂, —F, etc.

In some embodiments, L^{RG1} is a covalent bond. In some embodiments, L^{RG1} is not a covalent bond. In some embodiments, L^{RG1} is —S(O)₂—.

In some embodiments, L^{RG2} is —C(O)—. In some embodiments, a reactive group is or comprises -L^{LG4}-C(O)—, wherein each variable is as described herein. In some embodiments, a reactive group is or comprises -L^{LG3}-L^{LG4}-C(O)—, wherein each variable is as described herein. In some embodiments, a reactive group is or comprises -L^{LG2}-L^{LG3}-L^{LG4}-C(O)—, wherein each variable is as described herein.

In some embodiments, L^{RG2} is -L^{RG3}-C(═CR^{RG1}R^{RG2})—CR^{RG3}R^{RG4}—, wherein each of R^{RG1}, R^{RG2}, R^{RG3} and R^{RG4} is independently -L-R′, and L^{RG3} is —C(O)—, —C(O)O—, —C(O)N(R′)—, —S(O)—, —S(O)₂—, —P(O)(OR′)—, —P(O)(SR′)—, or —P(O)(N(R′)₂)—. In some embodiments, each of R^{RG1}, R^{RG2}, R^{RG3} and R^{RG4} is independently R′. In some embodiments, one or more of R^{RG1}, R^{RG2}, R^{RG3} and R^{RG4} is independently —H. In some embodiments, L^{RG3} is —C(O)—. In some embodiments, L^{RG3} is —C(O)O—. In some embodiments, —O—, —N(R′)—, etc. of L^{RG3} is bonded to L^{PM}.

In some embodiments, R^{RG1} is —H. In some embodiments, R^{RG3} is —H.

In some embodiments, L^{RG2} is optionally substituted -L^{RG3}-C(═CHR^{RG2})—CHR^{RG4}—, wherein each variable is as described herein.

In some embodiments, R^{RG2} and R^{RG4} are taken together with their intervening atoms to form an optionally substituted ring as described herein. In some embodiments, a formed ring is an optionally substituted 3-10 membered monocyclic or bicyclic ring having 0-5 heteroatoms. In some embodiments, a formed ring is an optionally substituted 3-10 membered cycloaliphatic ring. In some embodiments, a formed ring is an optionally substituted 3-8 membered cycloaliphatic ring. In some embodiments, a formed ring is an optionally substituted 5-8 membered cycloaliphatic ring. In some embodiments, a formed ring is an optionally substituted 5-membered cycloaliphatic ring. In some embodiments, a formed ring is an optionally substituted 6-membered cycloaliphatic ring. In some embodiments, a formed ring is an optionally substituted 7-membered cycloaliphatic ring. In some embodiments, a formed ring is substituted. In some embodiments, a formed ring is not substituted. In some embodiments, a formed ring contains no additional unsaturation in addition to the double bond in C(═CHR^{RG2}) or C(═CR^{RG1}R^{RG2}).

In some embodiments, —C(═CHR^{RG2})—CHR^{RG4} or —C(═CR^{RG1}R^{RG2})—CR^{RG3}R^{RG4} is optionally substituted

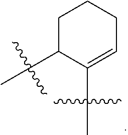 [SMILES: *C1=CCCCC1*]

That is —C(═CHR^{RG2})—CHR^{RG4} is a group of the formula

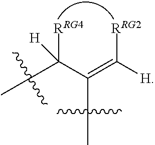

In some embodiments, —C(═CHR^{RG2})—CHR^{RG4} or —C(═CR^{RG1}R^{RG2})—CR^{RG3}R^{RG4} is

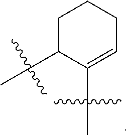 [SMILES: *C1=CCCCC1*]

In some embodiments, —[C(═CHR^{RG2})—CHR^{RG4}]-L^{RG3}- or —[C(═CR^{RG1}R^{RG2})—CR^{RG3}R^{RG4}]-L^{RG3}- is optionally substituted

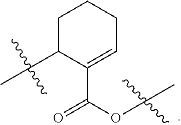 [SMILES: *OC(=O)C1=CCCCC1*]

In some embodiments, —[C(═CHR^{RG2})—CHR^{RG4}]-L^{RG3}- or —[C(═CR^{RG1}R^{RG2})—CR^{RG3}R^{RG4}]-L^{RG3}- is

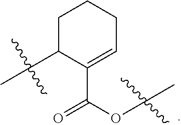 [SMILES: *OC(=O)C1=CCCCC1*]

In some embodiments, -L^{RG1}-[C(═CHR^{RG2})—CHR^{RG4}]-L^{RG3}- or -L^{RG1}-[C(═CR^{RG1}R^{RG2})—CR^{RG3}R^{RG4}]-L^{RG3}- is optionally substituted

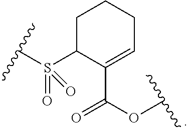 [SMILES: *OC(=O)C1=CCCCC1S(*)(=O)=O]

In some embodiments, -L^{RG1}-[C(═CHR^{RG2})—CHR^{RG4}]-L^{RG3}- or -L^{RG1}-[C(═CR^{RG1}R^{RG2})—CR^{RG3}R^{RG4}]-L^{RG3}- is optionally substituted

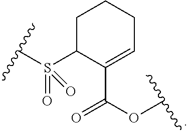 [SMILES: *OC(=O)C1=CCCCC1S(*)(=O)=O]

In some embodiments, a reactive group is a structure selected from the Table below. In some embodiments, -L^{LG2}-L^{LG3}-L^{LG4}-L^{RG1}-L^{RG2}- is a structure selected from Table below. In some embodiments, -L^{LG2}-L^{LG3}-L^{LG4}-L^{RG1}- is a structure selected from the Table below. Table RG-1. Certain structures as examples.

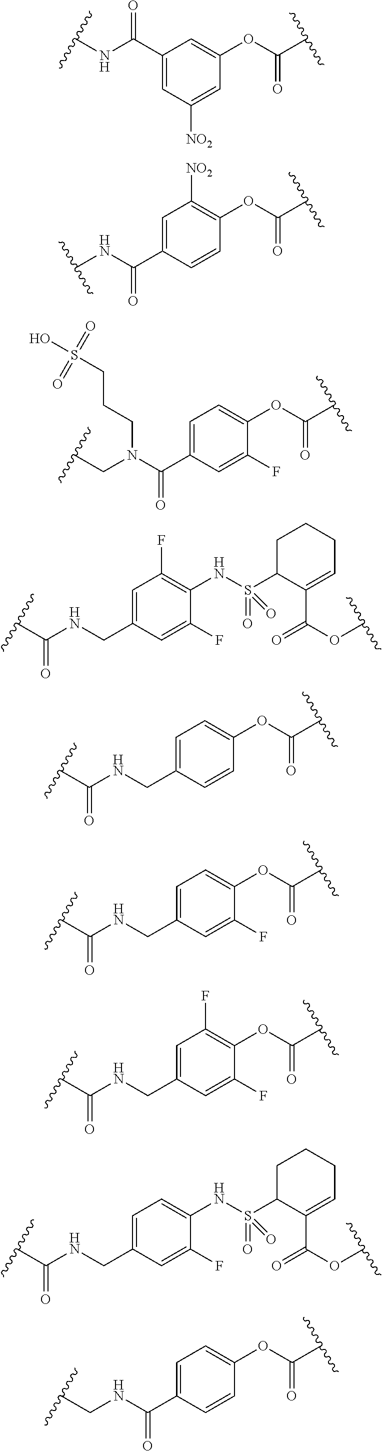

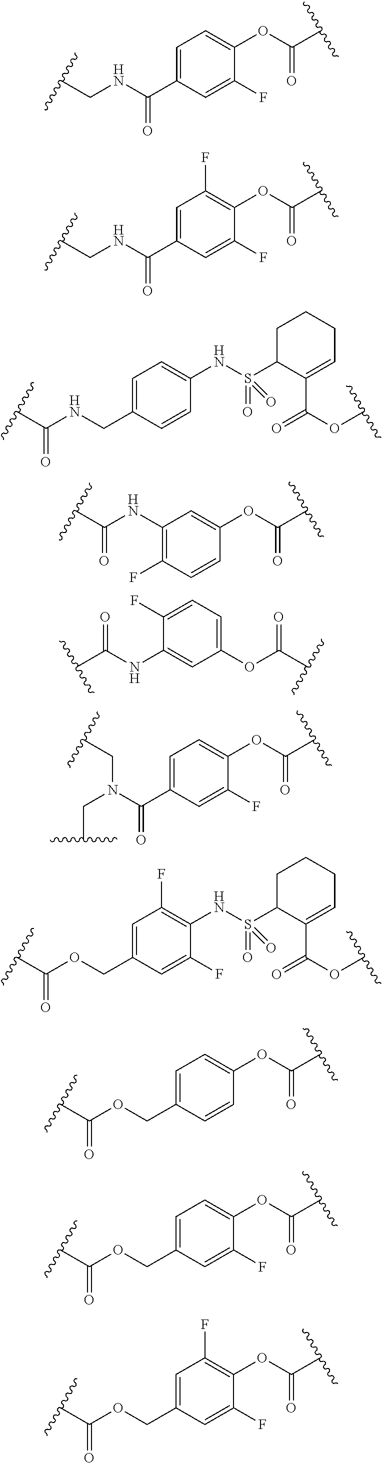

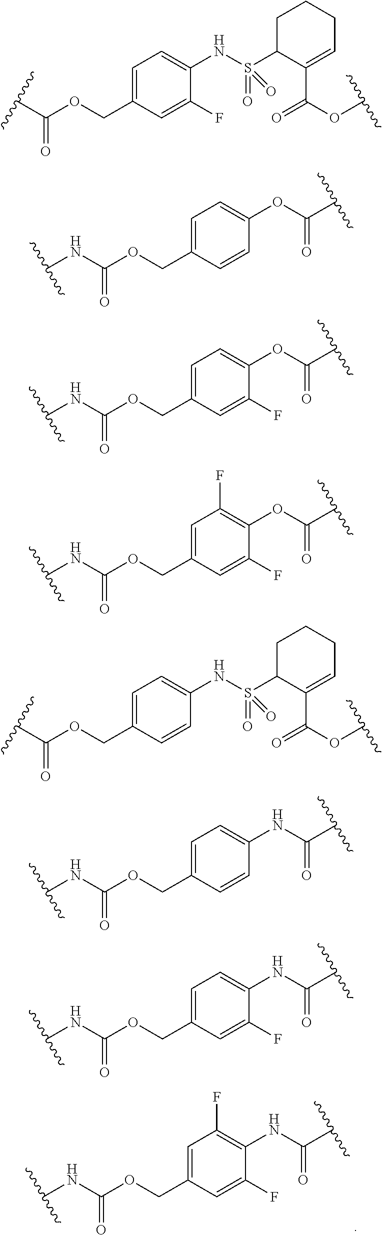

In some embodiments, -L^{LG4}-L^{RG2}- is —O—C(O)—. In some embodiments, -L^{LG4}-L^{RG2}- is —S—C(O)—. In some embodiments, -L^{LG4}-L^{RG1}-L^{RG2}- is —S—C(O)—.

In some embodiments, -L^{LG4}-L^{RG2}- is —N(−)—C(O)—, wherein N is a ring atom of an optionally substituted heteroaryl ring. In some embodiments, -L^{LG4}-L^{RG2}- is —N(−)—C(O)—, wherein N is a ring atom of L^{LG4} which is or comprises an optionally substituted heteroaryl ring. In some embodiments, -L^{LG4}-L^{RG2}- is —N(−)—C(O)—O—, wherein N is a ring atom of L^{LG4} which is or comprises an optionally substituted heteroaryl ring.

In some embodiments, L^{RG2} is optionally substituted —CH₂—C(O)—, wherein —CH₂— is bonded to an electron-withdrawing group comprising or connected to an antibody binding moiety. In some embodiments, L^{RG2} is optionally substituted —CH₂— bonded to an electron-withdrawing group comprising or connected to an antibody binding moiety. In some embodiments, L^{RG1} is an electron-withdrawing group. In some embodiments, L^{RG1} is —C(O)—. In some embodiments, L^{RG1} is —S(O)—. In some embodiments, L^{RG1} is —S(O)₂—. In some embodiments, L^{RG1} is —P(O(OR)—. In some embodiments, L^{RG1} is —P(O(SR)—. In some embodiments, L^{RG1} is —P(O(N(R)₂)—. In some embodiments, L^{RG1} is —OP(O(OR)—. In some embodiments, L^{RG1} is —OP(O(SR)—. In some embodiments, L^{RG1} is —OP(O(N(R)₂)—.

In some embodiments, L^{RG2} is optionally substituted —CH₂—C(O)—, wherein —CH₂— is bonded to a leaving group comprising or connected to an antibody binding moiety. In some embodiments, L^{RG2} is optionally substituted —CH₂— bonded to a leaving group comprising or connected to an antibody binding moiety. In some embodiments, L^{RG1} is —O—C(O)—. In some embodiments, L^{RG1} is —OS(O)₂—. In some embodiments, L^{RG1} is —OP(O(OR)—. In some embodiments, L^{RG1} is —OP(O(SR)—. In some embodiments, L^{RG1} is —OP(O(N(R)₂)—.

In some embodiments, a reactive group reacts with an amino group of a target agent. In some embodiments, an amino group is —NH₂ of the side chain of a lysine residue.

In some embodiments, a target agent is a protein agent. In some embodiments, a target agent is an antibody agent. In some embodiments, a reactive group reacts with an amino acid residue of such protein or antibody agent. In some embodiments, an amino acid residue is a lysine residue. In some embodiments, a reactive group reacts with —NH₂ of the side chain of a lysine residue. In some embodiments, a reactive group is or comprises —C(O)—O—, it reacts with —NH₂ (e.g., of the side chain of a lysine residue), and forms an amide group —C(O)—O— with the —NH₂.

In some embodiments, reactive groups, e.g., a first reactive group, a second reactive group, etc., are located at terminal locations. In some embodiments, agents such as first agents comprise first reactive groups linked to target binding moieties optionally through linker moieties, and do not contain antibody binding moieties.

In some embodiments, the present disclosure provides methods for preparing a composition comprising a plurality of agents, wherein each agent independently comprises:
an antibody moiety,
a target binding moiety, and
optionally a linker moiety linking an antibody moiety and a target binding moiety; which method comprise:
contacting a plurality of agents each of which independently comprises a reactive group with a plurality of antibody agents.

In some embodiments, an agent comprising a reactive group comprises an antibody binding moiety, a target binding moiety and optionally a linker. In some embodiments, agents comprising a reactive group share the same target binding moiety. In some embodiments, agents agent comprising a reactive group share the same structure. In some embodiments, antibody molecules are of such structures, properties and/or activities to provide antibody moieties in agents described herein. In some embodiments, a plurality of antibody molecules comprise two or more IgG subclasses. In some embodiments, a plurality of antibody molecules comprise IgG1. In some embodiments, a plurality of antibody molecules comprise IgG2. In some embodiments, a plurality of antibody molecules comprise IgG4. In some embodiments, a plurality of antibody molecules comprise IgG1 and IgG2. In some embodiments, a plurality of antibody molecules comprise IgG1, IgG2 and IgG4. In some embodiments, a plurality of antibody molecules comprise IgG1, IgG2, IgG3 and IgG4. In some embodiments, a plurality of antibody molecules are IVIG antibody molecules.

In some embodiments, provided agents comprise a reactive group, e.g.,

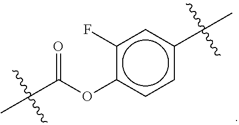

In some embodiments, —C(O)— is connected to a target binding moiety, or a moiety comprising -(Xaa)y-, optionally through a linker and the other end is connected to an antibody binding moiety. In some embodiments,

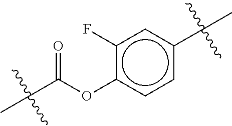

reacts with an amino group of another moiety, e.g., an antibody moiety, forming an amide group with the moiety and releasing a moiety which is or comprises antibody binding moiety. In some embodiments, an amino group is —NH₂ of a lysine side chain. In some embodiments, —C(O)— is connected to a target binding moiety, or a moiety comprising -(Xaa)y-, optionally through a linker and the other end is connected to R′ or an optional substituent. In some embodiments, provided agents comprise optionally substituted

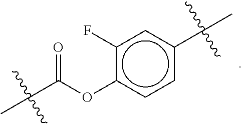

Such reactive groups may be useful for conjugation with detection, diagnosis or therapeutic agents. Those skilled in the art will appreciate that a variety of agents, and many technologies (e.g., click chemistry, reactions based on functional groups such as amino groups (e.g., amide formation), hydroxyl groups, carboxyl groups, etc.) can be utilized for conjugation in accordance with the present disclosure.

In some embodiments, antibody binding moieties bind to Fc regions of antibodies. In some embodiments, reactions occur at residues at Fc regions. In some embodiments, target binding moieties are conjugated to residues of Fc regions, optionally through linker moieties. In some embodiments, a residue is a Lys residue. In some embodiments, an antibody is or comprises IgG1. In some embodiments, an antibody is or comprises IgG2. In some embodiments, an antibody is or comprises IgG4. In some embodiments, an antibody composition utilized in a method comprises IgG1 and IgG2. In some embodiments, an antibody composition utilized in a method comprises IgG1, IgG2 and IgG4. In some embodiments, an antibody composition utilized in a method comprises IgG1, IgG2, IgG3 and IgG4.

In some embodiments, a product is or comprises IgG1. In some embodiments, a product is or comprises IgG2. In some embodiments, a product is or comprises IgG4. In some embodiments, a product composition comprises IgG1 and IgG2. In some embodiments, a product composition comprises IgG1, IgG2 and IgG4. In some embodiments, a product composition comprises IgG1, IgG2, IgG3 and IgG4.

In some embodiments, provided agents comprising antibody moieties provide one or more or substantially all antibody immune activities, e.g. for recruiting one or more types of immune cells and/or provide short-term and long-term immune activities. In some embodiments, provided agents comprising antibody moieties do not significantly reduce one or more or substantially all relevant antibody immune activities. In some embodiments, provided agents comprising antibody moieties improve one or more or substantially all relevant antibody immune activities (e.g., compared to antibody moieties by themselves). In some embodiments, provided agents provides comparable or better stability compared to antibody moieties by themselves (e.g., residence time in blood). In some embodiments, antibody moieties in provided agents can bind to FcRy of immune cells (e.g., various FcRy of immune effector cells for desired immune activities; typically at comparable or better levels). In some embodiments, antibody moieties in provided agents have comparable Fab/antigen binding capabilities. In some embodiments, antibody moieties in provided agents have comparable Fab/antigen binding capabilities. In some embodiments, antibody moieties in provided agents provide FcRn binding. In some embodiments, antibody moieties in provided agents provide FcRn binding, e.g., for antibody recycle and/or prolonged half-life. In some embodiments, provided technologies are particularly useful for modifying blood-derived IgG products as provided technologies are suitable for and can utilize all IgG subclasses.

In some embodiments, a provided method comprises one of the steps described below. In some embodiments,

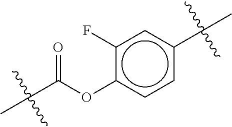

reacts with an amino group of a lysine side chain to form an amide bond with an antibody molecule, and releases

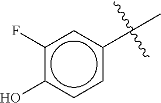

or a salt form thereof.

### Linker Moieties

In some embodiments, moieties are optionally connected to each other through linker moieties. For example, in some embodiments, a reactive group, e.g., RG, is connected to a moiety of interest, e.g., MOI, through a linker, e.g., L^{RM}. In some embodiments, a moiety, e.g., LG, may also comprise one or more linkers, e.g., L^{LG1}, L^{LG2}, L^{LG3}, L^{LG4}, etc., to link various portions. In some embodiments, L^{LG} is a linker moiety described herein. In some embodiments, L^{LG1} is a linker moiety described herein. In some embodiments, L^{LG2} is a linker moiety described herein. In some embodiments, L^{LG3} is a linker moiety described herein. In some embodiments, L^{LG4} is a linker moiety described herein. In some embodiments, L^{RM} is a linker moiety described herein. In some embodiments, L^{PM} is L as described herein. In some embodiments, L^{PM} is a linker moiety described herein. In some embodiments, L^{PM} is L as described herein.

Linker moieties of various types and/or for various purposes, e.g., those utilized in antibody-drug conjugates, etc., may be utilized in accordance with the present disclosure.

Linker moieties can be either bivalent or polyvalent depending on how they are used. In some embodiments, a linker moiety is bivalent. In some embodiments, a linker is polyvalent and connecting more than two moieties.

In some embodiments, a linker moiety, e.g., L^{z} (wherein z represents superscript text; e.g., L^{PM}, L^{RM}, L^{LG}, L^{LG1}, etc.), is or comprises L.

In some embodiments, L is a covalent bond, or a bivalent or polyvalent optionally substituted, linear or branched C₁₋₁₀₀ group comprising one or more aliphatic, aryl, heteroaliphatic having 1-20 heteroatoms, heteroaromatic having 1-20 heteroatoms, or any combinations thereof, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or —[(—O—C(R′)₂—C(R′)₂—)ₙ]—, wherein n is 1-20. The linker optionally contains a cyclic group, Cy, defined below, and a reactive group, RG as defined below. In some embodiments, each amino acid residue is independently a residue of an amino acid having the structure of formula A-I or a salt thereof. In some embodiments, each amino acid residue independently has the structure of —N(R^{a1})-L^{a1}-C(R^{a2})(R^{a3})-L^{a2}-CO— or a salt form thereof.

In some embodiments, L is bivalent. In some embodiments, L is a covalent bond.

In some embodiments, L is a bivalent or optionally substituted, linear or branched group selected from C₁₋₀₀ aliphatic and C₁₋₁₀₀ heteroaliphatic having 1-50 heteroatoms, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue or —[(—O—C(R′)₂—C(R′)₂—)ₙ]—. In some embodiments, L is a bivalent or optionally substituted, linear or branched group selected from C₁₋₂₀ aliphatic and C₁₋₂₀ heteroaliphatic having 1-10 heteroatoms, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue or —[(—O—C(R′)₂—C(R′)₂—)ₙ]—. In some embodiments, L is a bivalent or optionally substituted, linear or branched group selected from C₁₋₂₀ aliphatic wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue or —[(—O—C(R′)₂—C(R′)₂—)ₙ]—. In some embodiments, L is a bivalent or optionally substituted, linear or branched C₁₋₂₀ aliphatic wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, an amino acid residue or —[(—O—C(R′)₂—C(R′)₂—)ₙ]—. In some embodiments, L is a bivalent or optionally substituted, linear or branched group C₁₋₁₀₀ aliphatic wherein one or more methylene units of the group are optionally and independently replaced with —C≡C—, -Cy-, —C(R′)₂—, —O—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, an amino acid residue or —[(—O—C(R′)₂—C(R′)₂—)ₙ]—. In some embodiments, L is a bivalent or optionally substituted, linear or branched group C₁₋₅₀ aliphatic wherein one or more methylene units of the group are optionally and independently replaced as described herein. In some embodiments, L is a bivalent or optionally substituted, linear or branched group C₁₋₄₀ aliphatic wherein one or more methylene units of the group are optionally and independently replaced as described herein. In some embodiments, L is a bivalent or optionally substituted, linear or branched group C₁₋₂₀ aliphatic wherein one or more methylene units of the group are optionally and independently replaced as described herein. In some embodiments, L is a bivalent or optionally substituted, linear or branched group C₁₋₁₀ aliphatic wherein one or more methylene units of the group are optionally and independently replaced as described herein. In some embodiments, L is a bivalent or optionally substituted, linear or branched group C₁₋₁₀₀ alkylene wherein one or more methylene units of the group are optionally and independently replaced as described herein. In some embodiments, L is a bivalent or optionally substituted, linear or branched group C₁₋₅₀ alkylene wherein one or more methylene units of the group are optionally and independently replaced as described herein. In some embodiments, L is a bivalent or optionally substituted, linear or branched group C₁₋₄₀ alkylene wherein one or more methylene units of the group are optionally and independently replaced as described herein. In some embodiments, L is a bivalent or optionally substituted, linear or branched group C₁₋₂₀ alkylene wherein one or more methylene units of the group are optionally and independently replaced as described herein. In some embodiments, L is a bivalent or optionally substituted, linear or branched group C₁₋₁₀ alkylene wherein one or more methylene units of the group are optionally and independently replaced as described herein.

In some embodiments, a linker moiety, e.g., L, L^{PM}, L^{RM}, etc., comprises an acidic group, e.g., —S(O)₂OH.

In some embodiments, L is or comprises —[(—O—C(R′)₂—C(R′)₂—)ₙ]—. In some embodiments, L is or comprises —[(—O—CH₂—CH₂—),]-. In some embodiments, L is —[(—CH₂—CH₂—O)₆]—CH₂—CH₂—. In some embodiments, L is —[(—CH₂—CH₂—O)₈]—CH₂—CH₂—. In some embodiments, —CH₂—CH₂—O— is bonded to an antibody binding moiety at a —CH₂—. In some embodiments, —CH₂—CH₂—O— is bonded to a moiety of interest at a —CH₂—. In some embodiments, L^{PM} is such L as described herein. In some embodiments, L^{RM} is such L as described herein.

In some embodiments, a linker moiety, e.g., L, is or comprises, one or more —(CH2)n-O—, wherein each n is independently 1-20. In some embodiments, it is or comprises one or more —[(CH₂)n-O]m-, wherein each n is independently 1-20, and m is 1-100. In some embodiments, it comprises two or more —[(CH₂)n-O]m-, wherein each n is independently 1-20, and each m is 1-100. In some embodiments, it is or comprises one or more —(O)C—[(CH₂)nO]m(CH₂)nNH—, —[(CH₂)nO]mNHC(O)[(CH₂)nO]mNH—, —[(CH₂)nO]m{NHC(O)[(CH₂)nO]m}pNH—wherein each n is independently 1-20, and each m is independently 1-100, and where each p is independently 1 to 10. In some embodiments, n is 1-10. In some embodiments, n is 1-5. In some embodiments, each n is 2. In some embodiments, m is 1-50. In some embodiments, m is 1-40. In some embodiments, m is 1-30. In some embodiments, m is 1-20. In some embodiments, m is 1-10. In some embodiments. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, m is 12. In some embodiments, m is 13. In some embodiments, m is 14. In some embodiments, m is 15. In some embodiments, m is 16. In some embodiments, m is 17. In some embodiments, m is 18. In some embodiments, m is 19. In some embodiments, m is 20.

In some embodiments, a linker moiety, or L, is or comprises —(CH₂CH₂O)n-, wherein each —CH₂— is independently and optionally substituted and n is 1-20. In some embodiments, a linker moiety, or L, is or comprises —(CH₂)n-O—(CH₂CH₂O)n-(CH₂)n-, wherein each n is independently 1-10, and each —CH₂— is independently and optionally substituted.

In some embodiments, a linker moiety is trivalent or polyvalent. For example, in some embodiments, a linker moiety is L as described herein and L is trivalent or polyvalent. In some embodiments, L is trivalent. For example, in some embodiments, L is —CH₂—N(—CH₂—)—C(O)—.

In some embodiments, a linker moiety, e.g., L, comprises one or more amino acid residues or analogs thereof.

In some embodiments, a linker moiety, e.g., L, L^{RM}, etc., is or comprises a reactive group as described herein. In some embodiments, an agent comprises an antibody binding moiety and a target binding moiety linked through a linker which is or comprises a reactive group. In some embodiments, a reactive group can react with a lysine residue of an antibody in an aqueous buffer as described herein. In some embodiments, a reactive group is or comprises —C(O)—O—. In some embodiments, a reactive group is or comprises —C(O)—O—, wherein —O— is bonded to an optionally substituted aryl group. In some embodiments, a reactive group is or comprises —C(O)—O—, wherein —O— is bonded to an aryl group substituted with one or more electron-withdrawing groups. In some embodiments, one or more or each electron-withdrawing group is independently selected from —NO₂ and —F. In some embodiments, an aryl group has the structure of

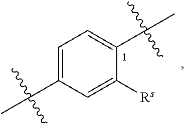

wherein R^{s} is halogen, —NO₂, —F, -L-R′, —C(O)-L-R′, —S(O)-L-R′, —S(O)₂-L-R′, or —P(O)(-L-R′)₂. In some embodiments, an aryl group has the structure of

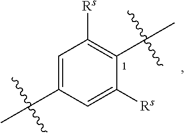

wherein each R^{s} is independently halogen, —NO₂, —F, -L-R′, —C(O)-L-R′, —S(O)-L-R′, —S(O)₂-L-R′, or —P(O)(-L-R′)₂. In some embodiments, an aryl group is

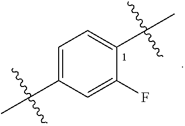 [SMILES: *c1ccc(*)c(F)c1]

In some embodiments, an aryl group is

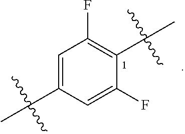 [SMILES: *c1cc(F)c(*)c(F)c1]

In some embodiments, Cl is bound to the —O— of —C(O)—O—. In some embodiments, a target binding moiety is at the side of —C(O)— and an antibody binding moiety is at the side of —O—.

In some embodiments, a linker moiety, e.g., L, L^{R}, etc., comprises a reactive group, wherein upon contact with an antibody, the reactive group reacts with a group of the antibody and conjugates a target binding moiety, or a moiety comprising -(Xaa)y-, to the antibody optionally through a linker. In some embodiments, a reactive group is or comprises

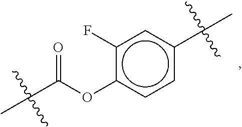

wherein the —C(O)— is connected to a target binding moiety, or a moiety comprising -(Xaa)y-, optionally through a linker. In some embodiments, a reactive group is or comprises

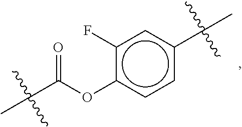

wherein the —C(O)— is connected to a target binding moiety, or a moiety comprising -(Xaa)y-, optionally through a linker and the other end of the reactive group is connected to an antibody binding moiety.

In some embodiments, L is or comprises a bioorthogonal or enzymatic reaction product moiety. In some embodiments, L is or comprise an optionally substituted triazole moiety (which is optionally part of a bi- or poly-cyclic ring system). In some embodiments, L is or comprises LPXTG. In some embodiments, L is or comprises LPETG. In some embodiments, L is or comprises LPXT(G)n, wherein n is 1, 2, 3, 4, 5, 6, 7, 8, 9, or 10. In some embodiments, L is or comprises LPET(G)n, wherein n is 1, 2, 3, 4, 5, 6, 7, 8, 9, or 10.

In some embodiments, a linker moiety, e.g., L, L^{RM}, etc., does not contain a reactive group. In some embodiments, a linker moiety, e.g., L, L^{RM}, etc., does not contain a reactive group that readily reacts with proteins under aqueous conditions with pH about 6-9 (e.g., physiological conditions). In some embodiments, a linker moiety, e.g., L, L^{RM}, etc., does not contain a reactive group that readily reacts with natural amino acid residues under aqueous conditions with pH about 6-9 (e.g., physiological conditions).

In some embodiments, a linker moiety, e.g., L, L^{RM}, etc., comprises no —S—, wherein none of the two atoms to which the —S— is bonded to is S. In some embodiments, a linker moiety, e.g., L, L^{RM}, etc., comprises no —S—S—. In some embodiments, a linker moiety, e.g., L, L^{RM}, etc., comprises no —S— bonded to a beta carbon of a carbonyl group or a double or triple bond conjugated to a carbonyl group. In some embodiments, a linker moiety, e.g., L, L^{RM}, etc., comprises no

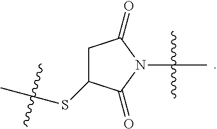 [SMILES: *SC1CC(=O)N(*)C1=O]

In some embodiments, a linker moiety, e.g., L, L^{RM}, etc., comprises no —S—.

In some embodiments, an agent comprises a linker which is not a covalent bond. In some embodiments, a linker has a length of (shortest path between linked moieties) about 1-200, 1-150, 1-100, 1-90, 1-80, 1-70, 1-60, 1-50, 1-40, 1-30, 10-200, 10-150, 10-100, 10-90, 10-80, 10-70, 10-60, 10-50, 10-40, 10-30, 20-200, 20-150, 20-100, 20-90, 20-80, 20-70, 20-60, 20-50, 20-40, 20-30, or about 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, or 200 atoms or bonds.

In some embodiments, provided compounds/agents (e.g., reaction partners, agents (e.g., products of provided methods and/or steps therein) comprise no cleavable groups (except one or more reactive groups and/or moieties therein) that could be cleaved under conditions that would not substantially damage or transform target agents and/or agents comprising target agent moieties (e.g., conjugation products comprising target agent moieties). In some embodiments, provided compounds/agents (e.g., reaction partners, agents (e.g., products of provided methods and/or steps therein) comprise no cleavable groups (except one or more reactive groups and/or moieties therein) that could be cleaved under conditions that would not render target agents and/or agents comprising target agent moieties (e.g., conjugation products comprising target agent moieties) for one or more uses (e.g., for use as diagnostic agents, therapeutic agents, etc.). In some embodiments, provided compounds/agents (e.g., reaction partners, agents (e.g., products of provided methods and/or steps therein) comprise no cleavable groups which can be cleaved under bioorthogonal conditions. In some embodiments, provided compounds/agents (e.g., reaction partners, agents (e.g., products of provided methods and/or steps therein) comprise no cleavable groups except those which can be cleaved without substantively damaging and/or transforming proteins. In some embodiments, a cleavable group is or comprises —S—, —S—S—, —S-Cy-, —C(O)—O—, —C(O)—S—, acetal moiety, —N═N—, imine moiety, —CH═N—, —P(O)(OR)O— moiety, —P(O)(OR)—N(R)— moiety, —C(O)—CH₂—C(COOH)═CHC(O)— moiety, —CHOH—CHOH— moiety, —Se— moiety, Si bonded to two oxygen atoms, —C(O)—CH₂— wherein the —CH₂— is bonded to a benzylic carbon wherein the phenyl ring of the benzyl group is substituted with —NO₂—, —C(O)—CH₂— wherein the —CH₂— is bonded to a benzylic carbon wherein the phenyl ring of the benzyl group is substituted with —NO₂— at o-position, or —C(O)—N(−)— moiety, wherein N is a ring atom of a heteroaryl ring. In some embodiments, a cleavable group is or comprises —S—S—, —S—CH₂—Cy-, —S-Cy-, —C(O)—O—, —C(O)—S—, acetal moiety, —N═N—, imine moiety, —CH═N—, —P(O)(OR)O— moiety, —P(O)(OR)—N(R)— moiety, —C(O)—CH₂—C(COOH)═CHC(O)— moiety, —CHOH—CHOH— moiety, —Se— moiety, Si bonded to two oxygen atoms, —C(O)—CH₂— wherein the —CH₂— is bonded to a benzylic carbon wherein the phenyl ring of the benzyl group is substituted with —NO₂—, —C(O)—CH₂— wherein the —CH₂— is bonded to a benzylic carbon wherein the phenyl ring of the benzyl group is substituted with —NO₂— at o-position, or —C(O)—N(−)— moiety, wherein N is a ring atom of a heteroaryl ring. In some embodiments, a cleavage group is a cleavable linker or a cleavable portion described in US 2020/0190165, the cleavable linkers and cleavable portions of each of which is incorporated herein by reference. In some embodiments, a cleavage group is:

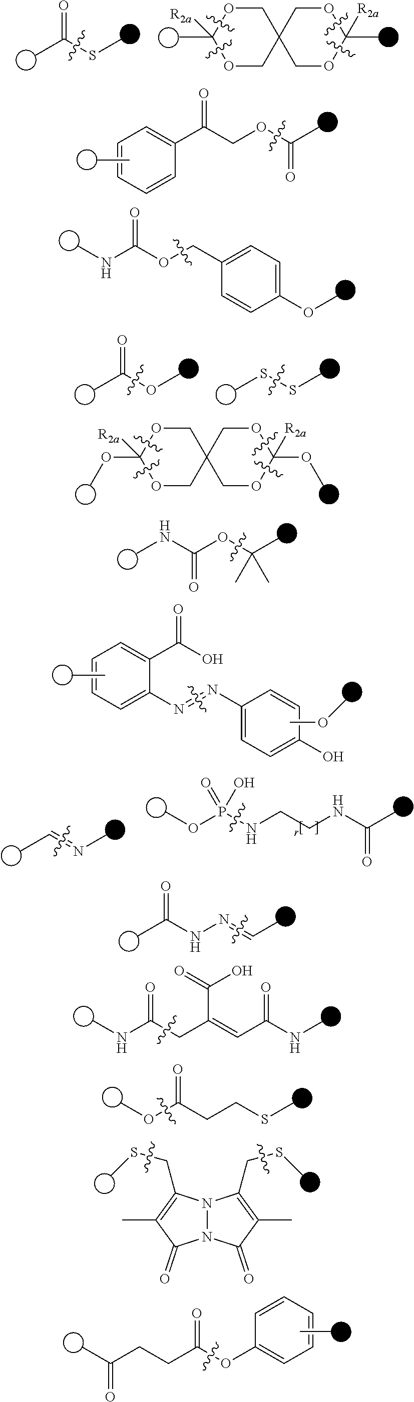

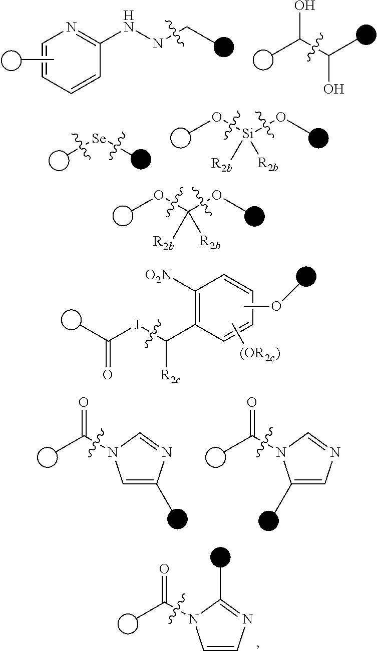

wherein:
a wavy line orthogonal to the bond indicates a potential cleavage site,
R^{2a}, R^{2b} and R^{2c} are the same or different and each is independently:
(i) a hydrogen atom or a halogen atom;
(ii) a monovalent hydrocarbon group;
(iii) aralkyl;
(iv) a monovalent heterocyclic group;
(v) R_{c}—O—, R_{c}—C(O)—, R_{c}—O—C(O)—, or R_{c}—C(O)—O—, wherein R_{c} is hydrogen or a monovalent hydrocarbon group;
(vi) —NR_{d}Rₑ, —NR_{d}Rₑ—C(O)—, —NR_{d}Rₑ—C(O)O—, —NR_{d}—C(O)—, —NR_{d}—C(O)O—, or R_{d}—C(O)—NRₑ—, wherein R_{d} and Rₑ are the same or different and each is a hydrogen atom or a monovalent hydrocarbon group; or
(vii) selected from a group consisting of a nitro group, a sulfuric acid group, a sulfonic acid group, a cyano group, and a carboxyl group;
J is —CH₂—, —O—, or —S—;
r is any integer of 1 to 4;
white circle and black circle are independently a bond connect to other moieties;

In some embodiments, a linker moiety does not contain a cleavage group as described above. In some embodiments, a linker moiety does not contain one or more or any of the following moieties: —S—, —S—S—, —S—CH₂—Cy-, —S-Cy-, —C(O)—O—, —C(O)—S—, acetal moiety, —N═N—, imine moiety, —CH═N—, —P(O)(OR)O— moiety, —P(O)(OR)—N(R)— moiety, —C(O)—CH₂—C(COOH)═CHC(O)— moiety, —CHOH—CHOH— moiety, —Se— moiety, Si bonded to two oxygen atoms, —C(O)—CH₂— wherein the —CH₂— is bonded to a benzylic carbon wherein the phenyl ring of the benzyl group is substituted with —NO₂—, —C(O)—CH₂— wherein the —CH₂— is bonded to a benzylic carbon wherein the phenyl ring of the benzyl group is substituted with —NO₂— at o-position, or —C(O)—N(−)— moiety, wherein N is a ring atom of a heteroaryl ring. In some embodiments, a linker moiety does not contain one or more or any of the following moieties: —S—S—, —S—CH₂—Cy-, —S-Cy-, —C(O)—O—, —C(O)—S—, acetal moiety, —N═N—, imine moiety, —CH═N—, —P(O)(OR)O— moiety, —P(O)(OR)—N(R)— moiety, —C(O)—CH₂—C(COOH)═CHC(O)— moiety, —CHOH—CHOH— moiety, —Se— moiety, Si bonded to two oxygen atoms, —C(O)—CH₂— wherein the —CH₂— is bonded to a benzylic carbon wherein the phenyl ring of the benzyl group is substituted with —NO₂—, —C(O)—CH₂— wherein the —CH₂— is bonded to a benzylic carbon wherein the phenyl ring of the benzyl group is substituted with —NO₂— at o-position, or —C(O)—N(−)— moiety, wherein N is a ring atom of a heteroaryl ring. In some embodiments, a linker moiety comprises no —S—. In some embodiments, a linker moiety comprises no —S—S— (optionally except a disulfide moiety formed by two amino acid residues, in some embodiments, optionally except a disulfide moiety formed by two cysteine residues). In some embodiments, a linker moiety comprises no —S-Cy-. In some embodiments, a linker moiety comprises no —S—CH₂—Cy-. In some embodiments, a linker moiety comprises no —C(O)—O—. In some embodiments, a linker moiety comprises no —C(O)—S—. In some embodiments, a linker moiety comprises no acetal moiety. In some embodiments, a linker moiety comprises no —N═N—. In some embodiments, a linker moiety comprises no imine moiety. In some embodiments, a linker moiety comprises no —CH═N— (optionally except in a ring, in some embodiments, optionally except in a heteroaryl ring). In some embodiments, a linker moiety comprises no —P(O)(OR)O— moiety. In some embodiments, a linker moiety comprises no —P(O)(OR)—N(R)— moiety. In some embodiments, a linker moiety comprises no —C(O)—CH₂—C(COOH)═CHC(O)— moiety. In some embodiments, a linker moiety comprises no —CHOH—CHOH— moiety. In some embodiments, a linker moiety comprises no —Se— moiety. In some embodiments, a linker moiety comprises no Si bonded to two oxygen atoms. In some embodiments, a linker moiety comprises no —C(O)—CH₂—, wherein the —CH₂— is bonded to a benzylic carbon, wherein the phenyl ring of the benzyl group is substituted with —NO₂—. In some embodiments, a linker moiety comprises no —C(O)—CH₂—, wherein the —CH₂— is bonded to a benzylic carbon, wherein the phenyl ring of the benzyl group is substituted with —NO₂— at o-position. In some embodiments, a linker moiety comprise no —C(O)—N(−)— moiety, wherein N is a ring atom of a heteroaryl ring. In some embodiments, a linker moiety does not contain any of these groups. In some embodiments, L^{RM} is such a linker moiety. In some embodiments, L^{PM} is such a linker moiety. In some embodiments, L^{LG} is such a linker moiety. In some embodiments, an agent of the present disclosure does not contain one or more or all of such moieties.

In some embodiments, an agent comprises no cleavable groups whose cleavage can release LG except one or more optionally in RG. In some embodiments, an agent comprises no —S—S—, acetal or imine groups except in RG or MOI. In some embodiments, an agent comprises no —S—S—, acetal or imine groups except that the agent may have —S—S— formed by two amino acid residues. In some embodiments, an agent comprises no —S—S—, acetal or imine groups except that the agent may have —S—S— formed by cysteine residues. In some embodiments, an agent comprises no —S—S—, acetal or imine groups.

In some embodiments, L is a covalent bond. In some embodiments, L is a bivalent optionally substituted, linear or branched C₁₋₁₀₀ aliphatic group wherein one or more methylene units of the group are optionally and independently replaced. In some embodiments, L is a bivalent optionally substituted, linear or branched C₆₋₁₀₀ arylaliphatic group wherein one or more methylene units of the group are optionally and independently replaced. In some embodiments, L is a bivalent optionally substituted, linear or branched C₅₋₁₀₀ heteroarylaliphatic group having 1-20 heteroatoms wherein one or more methylene units of the group are optionally and independently replaced. In some embodiments, L is a bivalent optionally substituted, linear or branched C₁₋₁₀₀ heteroaliphatic group having 1-20 heteroatoms wherein one or more methylene units of the group are optionally and independently replaced.

In some embodiments, a linker moiety (e.g., L) is or comprises one or more (e.g., 1, 2, 3, 4, 5, 6, 7, 8, 9, 10, 11, 12, 13, 14, 15, 16, 17, 18, 19, 20 or more) polyethylene glycol units. In some embodiments, a linker moiety is or comprises —(CH₂CH₂O)ₙ—, wherein n is as described in the present disclosure. In some embodiments, one or more methylene units of L are independently replaced with —(CH₂CH₂O)ₙ—.

As described herein, in some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, n is 11. In some embodiments, n is 12. In some embodiments, n is 13. In some embodiments, n is 14. In some embodiments, n is 15. In some embodiments, n is 16. In some embodiments, n is 17. In some embodiments, n is 18. In some embodiments, n is 19. In some embodiments, n is 20.

In some embodiments, a linker moiety (e.g., L) is or comprises one or more (e.g., 1, 2, 3, 4, 5, 6, 7, 8, 9, 10, 11, 12, 13, 14, 15, 16, 17, 18, 19, 20 or more) amino acid residues. As used in the present disclosure, “one or more” can be 1-100, 1-50, 1-40, 1-30, 1-20, 1-10, 1-5, 1, 2, 3, 4, 5, 6, 7, 8, 9, 10, 11, 12, 13, 14, 15, 16, 17, 18, 19, or 20 or more. In some embodiments, one or more methylene units of L are independently replaced with an amino acid residue. In some embodiments, one or more methylene units of L are independently replaced with an amino acid residue, wherein the amino acid residue is of an amino acid of formula A-I or a salt thereof. In some embodiments, one or more methylene units of L are independently replaced with an amino acid residue, wherein each amino acid residue independently has the structure of —N(R^{a1})-L^{a1}-C(R^{a2})(R^{a3})-L^{a2}-CO— or a salt form thereof.

In some embodiments, a linker moiety comprises one or more moieties, e.g., amino, carbonyl, etc., that can be utilized for connection with other moieties. In some embodiments, a linker moiety comprises one or more —NR′—, wherein R′ is as described in the present disclosure. In some embodiments, —NR′— improves solubility. In some embodiments, —NR′— serves as connection points to another moiety. In some embodiments, R′ is —H. In some embodiments, one or more methylene units of L are independently replaced with —NR′—, wherein R′ is as described in the present disclosure.

In some embodiments, a linker moiety, e.g., L, comprises a —C(O)— group, which can be utilized for connections with a moiety. In some embodiments, one or more methylene units of L are independently replaced with —C(O)—.

In some embodiments, a linker moiety, e.g., L, comprises a —NR′— group, which can be utilized for connections with a moiety. In some embodiments, one or more methylene units of L are independently replaced with —N(R′)—.

In some embodiments, a linker moiety, e.g., L, comprises a —C(O)NR′— group, which can be utilized for connections with a moiety. In some embodiments, one or more methylene units of L are independently replaced with —C(O)N(R′)—.

In some embodiments, a linker moiety, e.g., L, comprises a —C(R′)₂— group. In some embodiments, one or more methylene units of L are independently replaced with —C(R′)₂—. In some embodiments, —C(R′)₂— is —CHR′—. In some embodiments, R′ is —(CH₂)₂C(O)NH(CH₂)₁₁COOH. In some embodiments, R′ is —(CH₂)₂COOH. In some embodiments, R′ is —COOH.

In some embodiments, a linker moiety is or comprises one or more ring moieties, e.g., one or more methylene units of L are replaced with -Cy-. In some embodiments, a linker moiety, e.g., L, comprises an aryl ring. In some embodiments, a linker moiety, e.g., L, comprises an heteroaryl ring. In some embodiments, a linker moiety, e.g., L, comprises an aliphatic ring. In some embodiments, a linker moiety, e.g., L, comprises an heterocyclyl ring. In some embodiments, a linker moiety, e.g., L, comprises a polycyclic ring. In some embodiments, a ring in a linker moiety, e.g., L, is 3-20 membered. In some embodiments, a ring is 5-membered. In some embodiments, a ring is 6-membered. In some embodiments, a ring in a linker is product of a cycloaddition reaction (e.g., click chemistry, and variants thereof) utilized to link different moieties together.

In some embodiments, a linker moiety (e.g., L) is or comprises

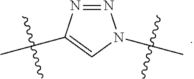 [SMILES: *c1cn(*)nn1]

In some embodiments, a methylene unit of L is replaced with

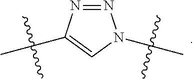 [SMILES: *c1cn(*)nn1]

In some embodiments, a methylene unit of L is replaced with -Cy-. In some embodiments, -Cy- is

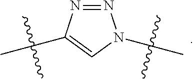 [SMILES: *c1cn(*)nn1]

In some embodiments, a linker moiety (e.g., L) is or comprises —CO)y-. In some embodiments L is or comprises —[(CH₂)nO]mCy[(CH₂)nO]mNH, or L is —[(CH₂)nO]mCy[(CH₂)nO]mNHC(O)[(CH₂)nO]mNH—, or L is —[(CH₂)nO]mCy[(CH₂)nO]m{NHC(O)[(CH₂)nO]m}pNH—, where n, m, and p are independently chosen at each occurrence from 1-20, from 1-12, or 2-10. In some embodiments each n is 2, m is independently chosen at each occurrence from an integer from 2-10, or in some embodiments m is independently chosen from an integer from 2-6 and Cy is

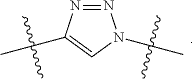 [SMILES: *c1cn(*)nn1]

In some embodiments, a methylene unit of L is replaced with -Cy-. In some embodiments, -Cy- is

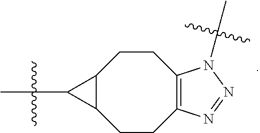 [SMILES: *C1C2CCc3nnn(*)c3CCC12]

In some embodiments, -Cy- is

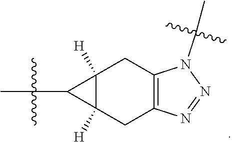 [SMILES: *C1[C@H]2Cc3c(nnn3*)C[C@@H]12]

In some embodiments, -Cy- is

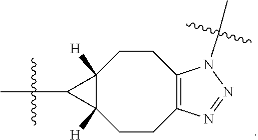 [SMILES: *C1[C@H]2CCc3nnn(*)c3CC[C@@H]12]

In some embodiments, a linker moiety, e.g., L, in a provided agent, e.g., a compound in Table 1, comprises

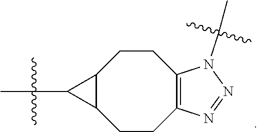 [SMILES: *C1C2CCc3nnn(*)c3CCC12]

In some embodiments,

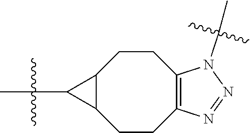 [SMILES: *C1C2CCc3nnn(*)c3CCC12]

is

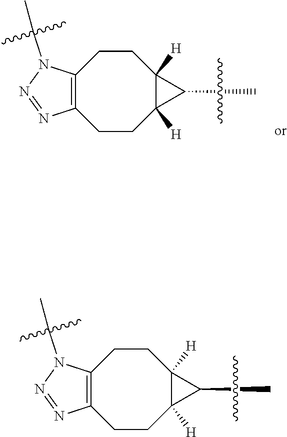

in the structure. In some embodiments,

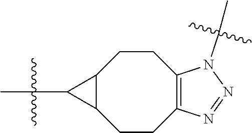 [SMILES: *C1C2CCc3nnn(*)c3CCC12]

is

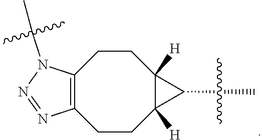

In some embodiments,

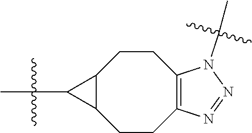 [SMILES: *C1C2CCc3nnn(*)c3CCC12]

is

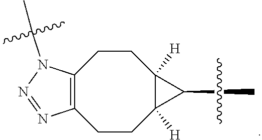

In some embodiments, a linker moiety is as described in Table 1. In some embodiments, L is L¹ as described in the present disclosure. In some embodiments, L is L^{b} as described in the present disclosure.

In some embodiments, L^{RM} is a covalent bond. In some embodiments, L^{RM} is not a covalent bond. In some embodiments, L^{RM} is or comprises —(CH₂CH₂O)n-. In some embodiments, L^{RM} is or comprises —(CH₂)n-O—(CH₂CH₂O)n-(CH₂)n-, wherein each n is independently as described herein, and each —CH₂— is independently optionally substituted. In some embodiments, L^{RM} is —(CH₂)n-O—(CH₂CH₂O)n-(CH₂)n-, wherein each n is independently as described herein, and each —CH₂— is independently optionally substituted. In some embodiments, L^{RM} is —(CH₂)₂—O—(CH₂CH₂O)n-(CH₂)₂—, wherein n is as described herein, and each —CH₂— is independently optionally substituted. In some embodiments, L^{RM} is —(CH₂)₂—O—(CH₂CH₂O)n-(CH₂)₂—, wherein n is as described herein.

In some embodiments, L^{PM} is a covalent bond. In some embodiments, L^{PM} is not a covalent bond. In some embodiments, L^{PM} is or comprises —(CH₂CH₂O)n-. In some embodiments, L^{PM} is or comprises —(CH₂)n-O—(CH₂CH₂O)n-(CH₂)n-, wherein each n is independently as described herein, and each —CH₂— is independently optionally substituted. In some embodiments, L^{PM} is —(CH₂)n-O—(CH₂CH₂O)n-(CH₂)n-, wherein each n is independently as described herein, and each —CH₂— is independently optionally substituted. In some embodiments, L^{PM} is —(CH₂)₂—O—(CH₂CH₂O)n-(CH₂)₂—, wherein n is as described herein, and each —CH₂— is independently optionally substituted. In some embodiments, L^{PM} is —(CH₂)₂—O—(CH₂CH₂O)n-(CH₂)₂—, wherein n is as described herein.

In some embodiments, L^{PM} (e.g., in a product of a first and a second agents) is or comprises a reaction product moiety formed a first reactive moiety and a second reactive moiety.

In some embodiments, a linker moiety (e.g., L^{PM} in a product of a first and a second agents) is or comprises

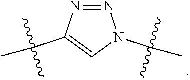 [SMILES: *c1cn(*)nn1]

In some embodiments, a methylene unit of a linker moiety, e.g., L or a linker moiety that can be L (e.g., L^{RM}, L^{PM}, etc.) is replaced with -Cy-. In some embodiments, -Cy- is optionally substituted

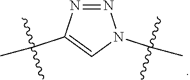 [SMILES: *c1cn(*)nn1]

In some embodiments, -Cy- is

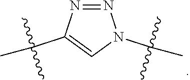 [SMILES: *c1cn(*)nn1]

In some embodiments L is —[(CH₂)nO]mCH₂Cy[(CH₂)nO]m- or In some embodiments, -Cy- is

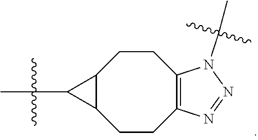 [SMILES: *C1C2CCc3nnn(*)c3CCC12]

In some embodiments, -Cy- is

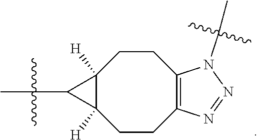 [SMILES: *C1[C@H]2CCc3c(nnn3*)CC[C@@H]12]

In some embodiments, -Cy- is

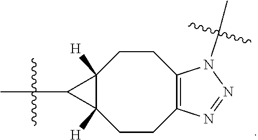 [SMILES: *C1[C@H]2CCc3nnn(*)c3CC[C@@H]12]

### Moieties of Interest

In some embodiments, a moiety of interest is a target binding moiety as described herein.

In some embodiments, moieties of interest are or comprise reactive groups, particularly those for bioorthogonal reactions. Suitable reactive moieties, including those for bioorthogonal reactions, are widely known in the art and can be utilized in accordance with the present disclosure. In some embodiments, a bioorthogonal reaction is a cycloaddition reaction, e.g., click chemistry. In some embodiments, a moiety of interest is or comprises —N₃. In some embodiments, a moiety of interest is or comprises an alkyne. In some embodiments, a moiety of interest is or comprises an alkyne suitable for metal-free click chemistry. For example, in some embodiments, a moiety of interest is or comprises optionally substituted

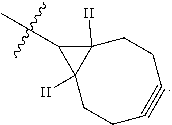 [SMILES: *C1C2CCC#CCCC12]

In some embodiments, a moiety of interest is or comprises

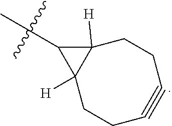 [SMILES: *C1C2CCC#CCCC12]

In some embodiments, a moiety of interest is or comprises

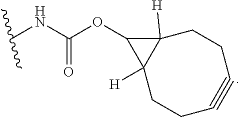 [SMILES: *NC(=O)OC1C2CCC#CCCC21]

In some embodiments, a moiety of interest is or comprises optionally substituted

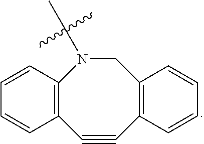 [SMILES: *N1Cc2ccccc2C#Cc2ccccc21]

In some embodiments, a moiety of interest is or comprises

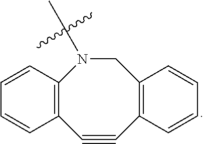 [SMILES: *N1Cc2ccccc2C#Cc2ccccc21]

In some embodiments, a moiety of interest is or comprises

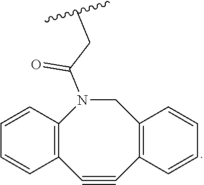 [SMILES: *CC(=O)N1Cc2ccccc2C#Cc2ccccc21]

In some embodiments, a moiety of interest is or comprises an aldehyde, ketone, alkoxyamine, or hydrazide moiety.

In some embodiments, a moiety of interest comprises a reactive group which is —SH or a salt form thereof.

In some embodiments, a moiety of interest is or comprises a peptide tag, e.g., for detection, transformation, reactions, etc. In some embodiments, a peptide tag is or comprises GGGGG (SEQ ID NO:84) and can serve as substrate for Sortase A mediated reaction with, e.g., LPETG (SEQ ID NO:85) tagged protein. In some embodiments, a peptide tag is or comprises LPXTG (SEQ ID NO:86). In some embodiments, a peptide tag is or comprises LPETG (SEQ ID NO:85). In some embodiments, a moiety of interest is or comprises (G)n, wherein n is 1-10. In some embodiments, a first G is the N-terminal residue. In some embodiments, a moiety of interest is or comprises LPXTG (SEQ ID NO:86), wherein X is an amino acid residue. In some embodiments, a moiety of interest is or comprises LPETG. In some embodiments, a moiety of interest is or comprises LPXTG-(X)n (SEQ ID NO:87), wherein each X is independently an amino acid residue, and n is 1-10. In some embodiments, a moiety of interest is or comprises LPETG-(X)n (SEQ ID NO:88), wherein each X is independently an amino acid residue, and n is 1-10. In some embodiments, n is 1, 2, 3, 4, 5, 6, 7, 8, 9 or 10. In some embodiments, n is 2. In some embodiments, n is 3. In some embodiments, n is 4. In some embodiments, n is 5. In some embodiments, n is 2-10. In some embodiments, n is 2-5. In some embodiments, n is 3-10. In some embodiments, n is 3-5.

In some embodiments, a moiety of interest is or comprises a reactive group (in some embodiments, is a tag for an, e.g., enzymatic reaction such as one promoted by a sortase). In some embodiments, such moiety of interests are useful for introducing second reactive groups to antibody moieties to provide second agents. In some embodiments, using provided technologies herein, reactive groups are selectively introduced to certain positions of antibody moieties, e.g., amino acid residues selected from K246 and K248 of an IgG1 heavy chain and amino acid residues corresponding thereto, K251 and K253 of an IgG2 heavy chain and amino acid residues corresponding thereto, and K239 and K241 of an IgG4 heavy chain and amino acid residues corresponding thereto.

In some embodiments, as described herein, a reaction between a first reactive group (e.g., of a first agent) and a second reactive group (e.g., of a second agent) is a bioorthogonal reaction. In some embodiments, a reaction is a cycloaddition reaction. In some embodiments, a reaction is a [3+2] reaction. Suitable such reactions and corresponding first and second reactive groups are widely known in the art and can be utilized in accordance with the present disclosure. In some embodiments, a first reactive group is or comprises —N₃, and a second reactive group is or comprises —C≡C— (e.g., an alkyne moiety suitable for click chemistry, including those suitable for metal-free click chemistry). In some embodiments, a second reactive group is or comprises —N₃, and a first reactive group is or comprises —C≡C— (e.g., an alkyne moiety suitable for click chemistry, including those suitable for metal-free click chemistry).

As described herein, in some embodiments, a reaction between a first reactive group and a second reactive group is an enzymatic reaction. In some embodiments, a reaction is a sortase-mediated reaction. In some embodiments, each of the first and second reactive group independently is or comprises a substrate moiety for a reaction, e.g., an enzymatic reaction. For example, in some embodiments, for a sortase-mediated conjugation, a first reactive group is or comprises (G)n (e.g., n is 3, 4, 5, etc.), and a second reactive group is or comprises LPXTG (SEQ ID NO:86)(e.g., LPETG, SEQ ID NO:85), or vice versa. In some embodiments, a first reactive group is or comprises (G)n (e.g., n is 3, 4, 5, etc.), and a second reactive group is or comprises LPXTG-(X)n (SEQ ID NO:87)(e.g., LPETG-(X)n (SEQ ID NO:88), LPETG-XX (SEQ ID NO:89), etc.) or vice versa. In some embodiments, a reactive group is or comprises ggsEQKLISEEDLGSGGGGSLPETGGggsggsSHFHHHHHHHH (SEQ ID NO:90). In some embodiments, a reactive group is or comprises GGG-. In some embodiments, a reactive group is or comprises GGGGG- (SEQ ID NO:84).

Those skilled in the art reading the present disclosure will appreciate that various reactive moieties can be utilized in accordance with the present disclosure for conjugation, via either enzymatic and/or non-enzymatic pathways.

In some embodiments, a linker moiety in a product agent comprises a reaction product moiety of a first and a second reactive groups. In some embodiments, L^{PM} of a product agent is or comprises a product moiety of a first reactive group and a second reactive group. For example, in some embodiments, when a reaction is or comprises a click reaction, L^{PM} in a product agent is or comprises a triazole moiety

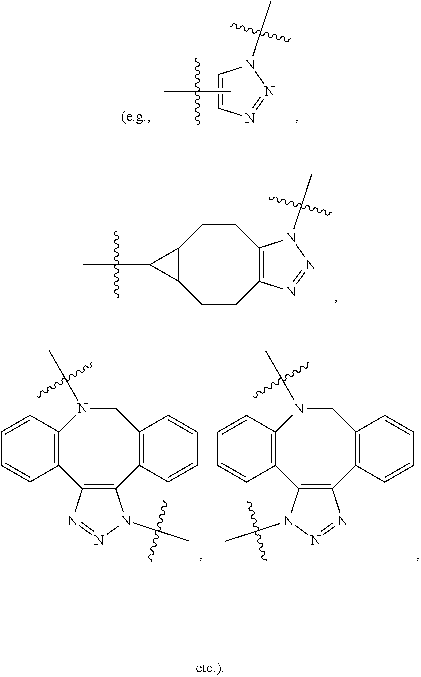

### Certain Embodiments of Variables

As examples, exemplified embodiments of variables are described throughout the present disclosure. As appreciated by those skilled in the art, embodiments for different variables may be optionally combined.

As defined above and described herein, ABT is an antibody binding moiety as described herein. In some embodiments, an ABT is a moiety selected from Table A-1. In some embodiments, an ABT is a moiety described in Table 1.

In some embodiments, L is a bivalent or multivalent linker moiety linking one or more antibody moieties with one or more target binding moieties. In some embodiments, L is a bivalent or multivalent linker moiety linking one or more antibody binding moieties with one or more target binding moieties. In some embodiments, L is a bivalent linker moiety that connects AT with TBT. In some embodiments, L is a multivalent linker moiety that connects AT with TBT. In some embodiments, L is a bivalent linker moiety that connects ABT with TBT. In some embodiments, L is a multivalent linker moiety that connects ABT with TBT.

In some embodiments, L is a linker moiety of a compound selected from those depicted in Table 1, below.

As defined above and described herein, AT is an antibody moiety as described herein.

As defined above and described herein, TBT is a target binding moiety as described herein.

In some embodiments, TBT is a target binding moiety of a compound selected from those depicted in Table 1, below. In some embodiments, an TBT is a moiety described in Table 1.

As defined above and described herein, each of R¹, R³ and R⁵ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or: R¹ and R^{1′} are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen, oxygen, or sulfur; R³ and R^{3′} are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen, oxygen, or sulfur; an R⁵ group and the R^{5′} group attached to the same carbon atom are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen, oxygen, or sulfur; or two R⁵ groups are optionally taken together with their intervening atoms to form a C₁₋₁₀ bivalent straight or branched saturated or unsaturated hydrocarbon chain wherein 1-3 methylene units of the chain are independently and optionally replaced with —S—, —SS—, —N(R)—, —O—, —C(O)—, —OC(O)—, —C(O)O—, —C(O)N(R)—, —N(R)C(O)—, —S(O)—, —S(O)₂—, or -Cy¹-, wherein each -Cy¹- is independently a 5-6 membered heteroarylenyl with 1-4 heteroatoms independently selected from nitrogen, oxygen or sulfur.

In some embodiments, R¹ is hydrogen. In some embodiments, R¹ is optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur. In some embodiments, R¹ is an optionally substituted C₁₋₆ aliphatic group. In some embodiments, R¹ is an optionally substituted 3-8 membered saturated or partially unsaturated monocyclic carbocyclic ring. In some embodiments, R¹ is an optionally substituted phenyl. In some embodiments, R¹ is an optionally substituted 8-10 membered bicyclic aromatic carbocyclic ring. In some embodiments, R¹ is an optionally substituted 4-8 membered saturated or partially unsaturated monocyclic heterocyclic ring having 1-2 heteroatoms independently selected from nitrogen, oxygen, or sulfur. In some embodiments, R¹ is an optionally substituted 5-6 membered monocyclic heteroaromatic ring having 1-4 heteroatoms independently selected from nitrogen, oxygen, or sulfur. In some embodiments, R¹ is an optionally substituted 8-10 membered bicyclic heteroaromatic ring having 1-5 heteroatoms independently selected from nitrogen, oxygen, or sulfur.

In some embodiments, R¹ is

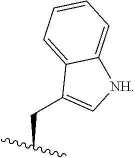

In some embodiments, R¹ is

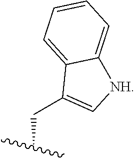

In some embodiments, R¹ is

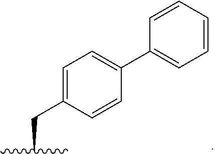

In some embodiments, R¹ is

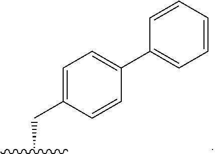

In some embodiments, R¹ is

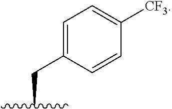

In some embodiments, R¹ is

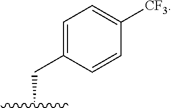

In some embodiments, R¹ is

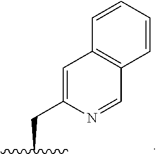

In some embodiments, R¹ is

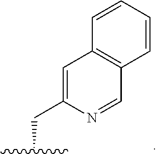

In some embodiments, R¹ is

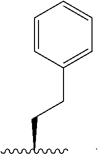

In some embodiments, R¹ is

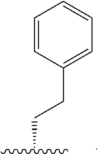

In some embodiments, R¹ is

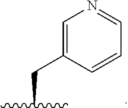

In some embodiments, R¹ is

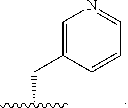

In some embodiments, R¹ is

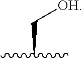

In some embodiments, R¹ is

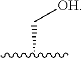

In some embodiments, R¹ is

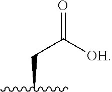

In some embodiments, R¹ is

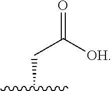

In some embodiments, R¹ is

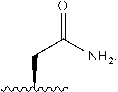

In some embodiments, R¹ is

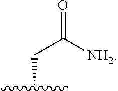

In some embodiments, R¹ is

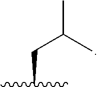

In some embodiments, R¹ is

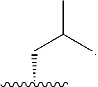

In some embodiments, R¹ and R^{1′} are optionally taken together with their intervening carbon atom to form a 3-8 membered saturated or partially unsaturated spirocyclic carbocyclic ring. In some embodiments, R¹ and R^{1′} are optionally taken together with their intervening carbon atom to form a 4-8 membered saturated or partially unsaturated spirocyclic heterocyclic ring having 1-2 heteroatoms independently selected from nitrogen, oxygen, or sulfur.

In some embodiments, R is R¹ as described in the present disclosure. In some embodiments, R² is R¹ as described in the present disclosure. In some embodiments, R³ is R¹ as described in the present disclosure.

In some embodiments, R³ is hydrogen. In some embodiments, R³ is optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur. In some embodiments, R³ is an optionally substituted C₁₋₆ aliphatic group. In some embodiments, R³ is an optionally substituted 3-8 membered saturated or partially unsaturated monocyclic carbocyclic ring. In some embodiments, R³ is an optionally substituted phenyl. In some embodiments, R³ is an optionally substituted 8-10 membered bicyclic aromatic carbocyclic ring. In some embodiments, R³ is an optionally substituted 4-8 membered saturated or partially unsaturated monocyclic heterocyclic ring having 1-2 heteroatoms independently selected from nitrogen, oxygen, or sulfur. In some embodiments, R³ is an optionally substituted 5-6 membered monocyclic heteroaromatic ring having 1-4 heteroatoms independently selected from nitrogen, oxygen, or sulfur. In some embodiments, R³ is an optionally substituted 8-10 membered bicyclic heteroaromatic ring having 1-5 heteroatoms independently selected from nitrogen, oxygen, or sulfur.

In some embodiments, R³ is methyl. In some embodiments, R³ is

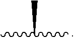

In some embodiments, R³ is

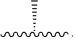

In some embodiments, R³ is

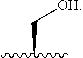

In some embodiments, R³ is

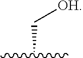

In some embodiments, R³ is

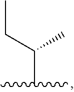 [SMILES: *[C@@H](C)CC]

wherein the site of attachment has (S) stereochemistry. In some embodiments, R³ is

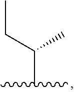 [SMILES: *[C@@H](C)CC]

wherein the site of attachment has (R) stereochemistry. In some embodiments, R³ is

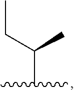 [SMILES: *[C@H](C)CC]

wherein the site of attachment has (S) stereochemistry. In some embodiments, R³ is

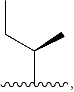 [SMILES: *[C@H](C)CC]

wherein the site of attachment has (R) stereochemistry.

In some embodiments, R³ is

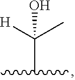 [SMILES: *[C@@H](C)O]

wherein the site of attachment has (S) stereochemistry. In some embodiments, R³ is

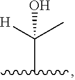 [SMILES: *[C@@H](C)O]

wherein the site of attachment has (R) stereochemistry.

In some embodiments, R³ and R^{3′} are optionally taken together with their intervening carbon atom to form a 3-8 membered saturated or partially unsaturated spirocyclic carbocyclic ring. In some embodiments, R³ and R^{3′} are optionally taken together with their intervening carbon atom to form a 4-8 membered saturated or partially unsaturated spirocyclic heterocyclic ring having 1-2 heteroatoms independently selected from nitrogen, oxygen, or sulfur.

In some embodiments, R is R² as described in the present disclosure. In some embodiments, R² is R² as described in the present disclosure. In some embodiments, R³ is R² as described in the present disclosure.

In some embodiments, R⁵ is hydrogen. In some embodiments, R⁵ is optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur. In some embodiments, R⁵ is an optionally substituted C₁₋₆ aliphatic group. In some embodiments, R⁵ is an optionally substituted 3-8 membered saturated or partially unsaturated monocyclic carbocyclic ring. In some embodiments, R⁵ is an optionally substituted phenyl. In some embodiments, R⁵ is an optionally substituted 8-10 membered bicyclic aromatic carbocyclic ring. In some embodiments, R⁵ is an optionally substituted 4-8 membered saturated or partially unsaturated monocyclic heterocyclic ring having 1-2 heteroatoms independently selected from nitrogen, oxygen, or sulfur. In some embodiments, R⁵ is an optionally substituted 5-6 membered monocyclic heteroaromatic ring having 1-4 heteroatoms independently selected from nitrogen, oxygen, or sulfur. In some embodiments, R⁵ is an optionally substituted 8-10 membered bicyclic heteroaromatic ring having 1-5 heteroatoms independently selected from nitrogen, oxygen, or sulfur.

In some embodiments, R⁵ is methyl. In some embodiments, R is

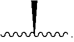

In some embodiments, R⁵ is

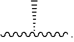

In some embodiments, R⁵ is

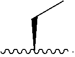

In some embodiments, R⁵ is

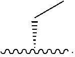

In some embodiments, R⁵ is

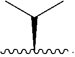

In some embodiments, R⁵ is

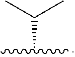

In some embodiments, R⁵ is

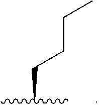

In some embodiments, R⁵ is

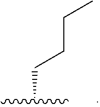

In some embodiments, R⁵ is

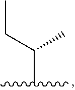 [SMILES: *[C@@H](C)CC]

wherein the site of attachment has (S) stereochemistry. In some embodiments, R⁵ is

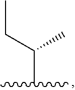 [SMILES: *[C@@H](C)CC]

wherein the site of attachment has (R) stereochemistry. In some embodiments, R⁵ is

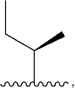 [SMILES: *[C@H](C)CC]

wherein the site of attachment has (S) stereochemistry. In some embodiments, R⁵ is

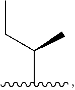 [SMILES: *[C@H](C)CC]

wherein the site of attachment has (R) stereochemistry. In some embodiments, R⁵ is

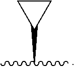

In some embodiments, R⁵ is

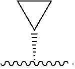

In some embodiments, R⁵ is

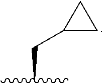

In some embodiments, R⁵ is

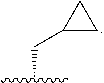

In some embodiments, R⁵ is

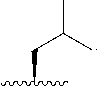

In some embodiments, R⁵ is

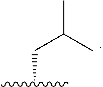

In some embodiments, R⁵ is

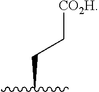

In some embodiments, R⁵ is

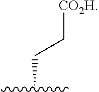

In some embodiments, R⁵ is

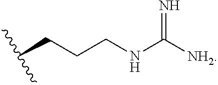

In some embodiments, R⁵ is

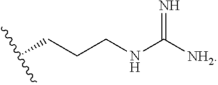

In some embodiments R⁵ is

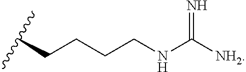

In some embodiments, R⁵ is

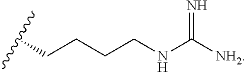

In some embodiments, R⁵ is

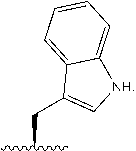

In some embodiments, R⁵ is

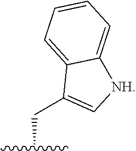

In some embodiments, R⁵ is

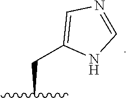

In some embodiments, R⁵ is

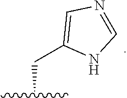

In some embodiments, R⁵ is

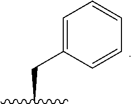

In some embodiments, R⁵ is

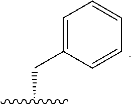

In some embodiments, R⁵ is

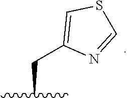

In some embodiments, R⁵ is

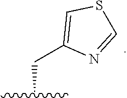

In some embodiments, R⁵ is

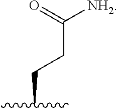

In some embodiments, R⁴ is5

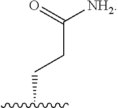

In some embodiments, R⁵ is

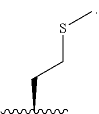

In some embodiments, R⁵ is

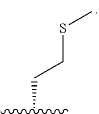

In some embodiments, R⁵ is

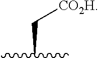

In some embodiments, R⁵ is

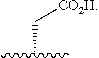

In some embodiments, R⁴ is

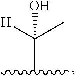 [SMILES: *[C@@H](C)O]

wherein the site of attachment has (S) stereochemistry. In some embodiments, R⁴ is

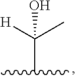 [SMILES: *[C@@H](C)O]

wherein the site of attachment has (R) stereochemistry.

In some embodiments, R⁵ and the R′ group attached to the same carbon atom are optionally taken together with their intervening carbon atom to form a 3-8 membered saturated or partially unsaturated spirocyclic carbocyclic ring. In some embodiments, R⁵ and the R^{5′} group attached to the same carbon atom are optionally taken together with their intervening carbon atom to form a 4-8 membered saturated or partially unsaturated spirocyclic heterocyclic ring having 1-2 heteroatoms independently selected from nitrogen, oxygen, or sulfur.

In some embodiments, two R⁵ groups are taken together with their intervening atoms to form a C₁₋₁₀ bivalent straight or branched saturated or unsaturated hydrocarbon chain wherein 1-3 methylene units of the chain are independently and optionally replaced with —S—, —SS—, —N(R)—, —O—, —C(O)—, —OC(O)—, —C(O)O—, —C(O)N(R)—, —N(R)C(O)—, —S(O)—, —S(O)₂—, or -Cy¹-, wherein each -Cy¹- is independently a 5-6 membered heteroarylenyl with 1-4 heteroatoms independently selected from nitrogen, oxygen or sulfur.

In some embodiments, two R⁵ groups are taken together with their intervening atoms to form

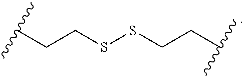 [SMILES: *CCSSCC*]

In some embodiments, two R⁵ groups are taken together with their intervening atoms to form

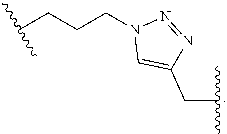 [SMILES: *CCCn1cc(C*)nn1]

In some embodiments, two R⁵ groups are taken together with their intervening atoms to form

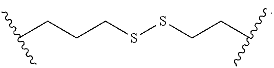 [SMILES: *CCCSSCC*]

In some embodiments, two R⁵ groups are taken together with their intervening atoms to form

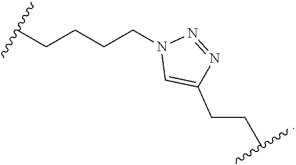 [SMILES: *CCCCn1cc(CC*)nn1]

In some embodiments, R is R⁵ as described in the present disclosure. In some embodiments, R^{a2} is R⁵ as described in the present disclosure. In some embodiments, R^{a3} is R⁵ as described in the present disclosure.

As defined above and described herein, each of R^{1′}, R^{3′} and R^{5′} is independently hydrogen or C₁₋₃ aliphatic.

In some embodiments, R^{1′} is hydrogen. In some embodiments, R^{1′} is C₁₋₃ aliphatic.

In some embodiments, R^{1′} is methyl. In some embodiments, R^{1′} is ethyl. In some embodiments, R^{1′} is n-propyl. In some embodiments, R^{1′} is isopropyl. In some embodiments, Rr is cyclopropyl.

In some embodiments, R^{3′} is hydrogen. In some embodiments, R^{3′} is C₁₋₃ aliphatic.

In some embodiments, R^{3′} is methyl. In some embodiments, R^{3′} is ethyl. In some embodiments, R^{3′} is n-propyl. In some embodiments, R^{3′} is isopropyl. In some embodiments, R^{3′} is cyclopropyl.

In some embodiments, R^{5′} is hydrogen. In some embodiments, R^{5′} is C₁₋₃ aliphatic.

In some embodiments, R^{5′} is methyl. In some embodiments, R⁵ is ethyl. In some embodiments, R^{5′} is n-propyl. In some embodiments, R^{5′} is isopropyl. In some embodiments, R^{5′} is cyclopropyl.

As defined above and described herein, each of R², R⁴ and R⁶ is independently hydrogen, or C₁₋₄ aliphatic, or: R² and R¹ are optionally taken together with their intervening atoms to form a 4-8 membered saturated or partially unsaturated monocyclic heterocyclic ring having 1-2 heteroatoms independently selected from nitrogen, oxygen, or sulfur; R⁴ and R³ are optionally taken together with their intervening atoms to form a 4-8 membered saturated or partially unsaturated monocyclic heterocyclic ring having 1-2 heteroatoms independently selected from nitrogen, oxygen, or sulfur; or an R⁶ group and its adjacent R⁵ group are optionally taken together with their intervening atoms to form a 4-8 membered saturated or partially unsaturated monocyclic heterocyclic ring having 1-2 heteroatoms independently selected from nitrogen, oxygen, or sulfur.

In some embodiments, R² is hydrogen. In some embodiments, R² is C₁₋₄ aliphatic. In some embodiments, R² is methyl. In some embodiments, R² is ethyl. In some embodiments, R² is n-propyl. In some embodiments, R² is isopropyl. In some embodiments, R² is n-butyl. In some embodiments, R² is isobutyl. In some embodiments, R² is tert-butyl.

In some embodiments, R² and R¹ are taken together with their intervening atoms to form a 4-8 membered saturated or partially unsaturated monocyclic heterocyclic ring having 1-2 heteroatoms independently selected from nitrogen, oxygen, or sulfur.

In some embodiments, R² and R¹ are taken together with their intervening atoms to form

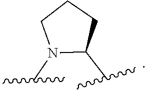 [SMILES: *[C@@H]1CCCN1*]

In some embodiments, R² and R¹ are taken together with their intervening atoms to form

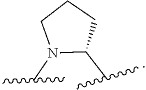 [SMILES: *[C@H]1CCCN1*]

In some embodiments, R⁴ is hydrogen. In some embodiments, R⁴ is C₁₋₄ aliphatic. In some embodiments, R⁴ is methyl. In some embodiments, R⁴ is ethyl. In some embodiments, R⁴ is n-propyl. In some embodiments, R⁴ is isopropyl. In some embodiments, R⁴ is n-butyl. In some embodiments, R⁴ is isobutyl. In some embodiments, R⁴ is tert-butyl.

In some embodiments, R⁴ and R³ are taken together with their intervening atoms to form a 4-8 membered saturated or partially unsaturated monocyclic heterocyclic ring having 1-2 heteroatoms independently selected from nitrogen, oxygen, or sulfur.

In some embodiments, R⁴ and R³ are taken together with their intervening atoms to form

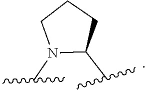 [SMILES: *[C@@H]1CCCN1*]

In some embodiments, R⁴ and R³ are taken together with their intervening atoms to form

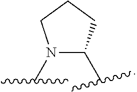 [SMILES: *[C@H]1CCCN1*]

In some embodiments, R⁶ is hydrogen. In some embodiments, R⁶ is C₁₋₄ aliphatic. In some embodiments, R⁶ is methyl. In some embodiments, R⁶ is ethyl. In some embodiments, R⁶ is n-propyl. In some embodiments, R⁶ is isopropyl. In some embodiments, R⁶ is n-butyl. In some embodiments, R⁶ is isobutyl. In some embodiments, R⁶ is tert-butyl.

In some embodiments, an R⁶ group and its adjacent R⁵ group are taken together with their intervening atoms to form a 4-8 membered saturated or partially unsaturated monocyclic heterocyclic ring having 1-2 heteroatoms independently selected from nitrogen, oxygen, or sulfur.

In some embodiments, an R⁶ group and its adjacent R⁵ group are taken together with their intervening atoms to form

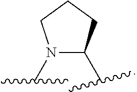 [SMILES: *[C@@H]1CCCN1*]

In some embodiments, an R⁶ group and its adjacent R⁵ group are taken together with their intervening atoms to form

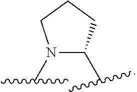 [SMILES: *[C@H]1CCCN1*]

In some embodiments, R is R^{1′} as described in the present disclosure. In some embodiments, R^{a2} is R^{1′} as described in the present disclosure. In some embodiments, R^{a3} is R^{1′} as described in the present disclosure. In some embodiments, R is R^{3′} as described in the present disclosure. In some embodiments, R^{a2} is R^{3′} as described in the present disclosure. In some embodiments, R^{a3} is R^{3′} as described in the present disclosure. In some embodiments, R is R² as described in the present disclosure. In some embodiments, R^{a2} is R² as described in the present disclosure. In some embodiments, R^{a3} is R² as described in the present disclosure. In some embodiments, R is R⁴ as described in the present disclosure. In some embodiments, R^{a2} is R⁴ as described in the present disclosure. In some embodiments, R^{a3} is R⁴ as described in the present disclosure. In some embodiments, R is R⁶ as described in the present disclosure. In some embodiments, R^{a2} is R⁶ as described in the present disclosure. In some embodiments, R^{a3} is R⁶ as described in the present disclosure.

As defined above and described herein, L¹ is a trivalent linker moiety that connects

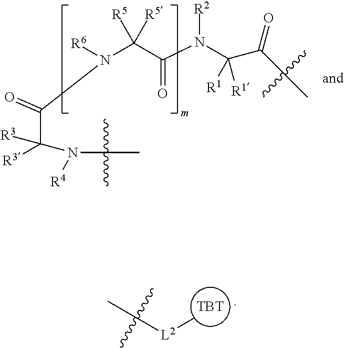

In some embodiments, L¹ is

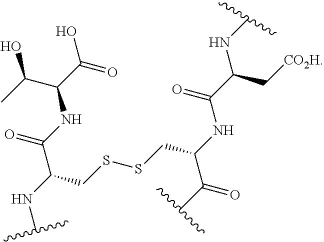 [SMILES: *N[C@@H](CC(=O)O)C(=O)N[C@@H](CSSC[C@H](N*)C(=O)N[C@H](C(=O)O)[C@@H](C)O)C(*)=O]

In some embodiments, L¹ is

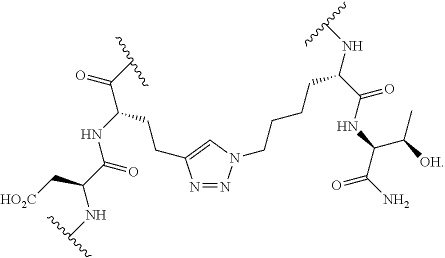 [SMILES: *N[C@@H](CC(=O)O)C(=O)N[C@@H](CCc1cn(CCCC[C@H](N*)C(=O)N[C@H](C(N)=O)[C@@H](C)O)nn1)C(*)=O]

In some embodiments, L¹ is

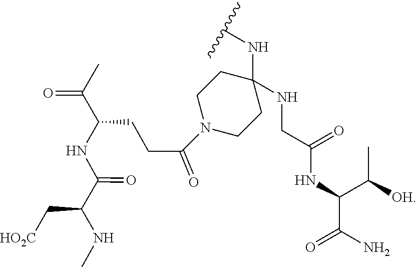 [SMILES: *NC1(NCC(=O)N[C@H](C(N)=O)[C@@H](C)O)CCN(C(=O)CC[C@H](NC(=O)[C@H](CC(=O)O)NC)C(C)=O)CC1]

In some embodiments, L³ is

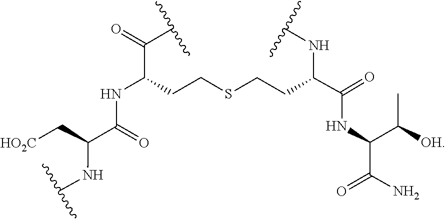 [SMILES: *N[C@@H](CC(=O)O)C(=O)N[C@@H](CCSCC[C@H](N*)C(=O)N[C@H](C(N)=O)[C@@H](C)O)C(*)=O]

In some embodiments, L¹ is

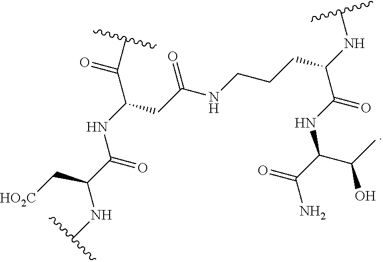 [SMILES: *N[C@@H](CC(=O)O)C(=O)N[C@@H](CC(=O)NCCC[C@H](N*)C(=O)N[C@H](C(N)=O)[C@@H](C)O)C(*)=O]

In some embodiments, L¹ is

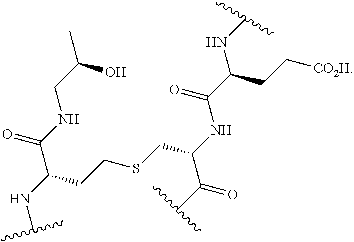 [SMILES: *N[C@@H](CCSC[C@H](NC(=O)[C@H](CCC(=O)O)N*)C(*)=O)C(=O)NC[C@@H](C)O]

In some embodiments, L¹ is

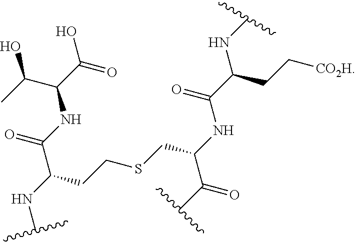 [SMILES: *N[C@@H](CCC(=O)O)C(=O)N[C@@H](CSCC[C@H](N*)C(=O)N[C@H](C(=O)O)[C@@H](C)O)C(*)=O]

In some embodiments, L¹ is

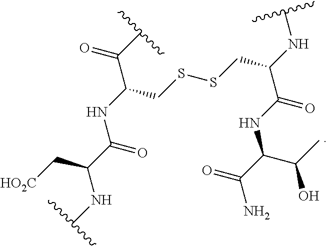 [SMILES: *N[C@@H](CC(=O)O)C(=O)N[C@@H](CSSC[C@H](N*)C(=O)N[C@H](C(N)=O)[C@@H](C)O)C(*)=O]

In some embodiments, L¹ is

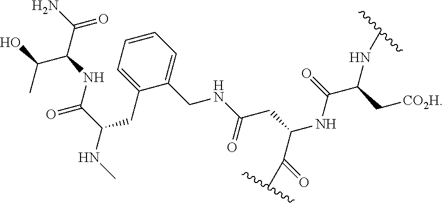 [SMILES: *N[C@@H](CC(=O)O)C(=O)N[C@@H](CC(=O)NCc1ccccc1C[C@H](NC)C(=O)N[C@H](C(N)=O)[C@@H](C)O)C(*)=O]

In some embodiments, L¹ is

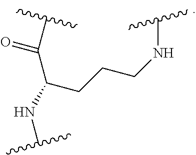 [SMILES: *NCCC[C@H](N*)C(*)=O]

In some embodiments, L¹ is

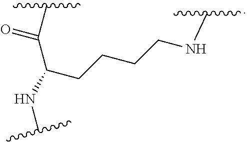 [SMILES: *NCCCC[C@H](N*)C(*)=O]

In some embodiments, L¹ is

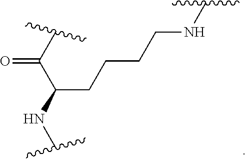 [SMILES: *NCCCC[C@@H](N*)C(*)=O]

In some embodiments, L¹ is

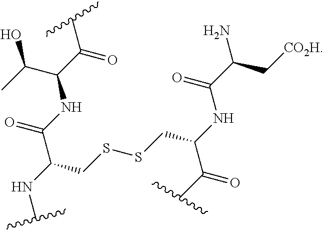 [SMILES: *N[C@@H](CSSC[C@H](NC(=O)[C@@H](N)CC(=O)O)C(*)=O)C(=O)N[C@H](C(*)=O)[C@@H](C)O]

In some embodiments, L¹ is

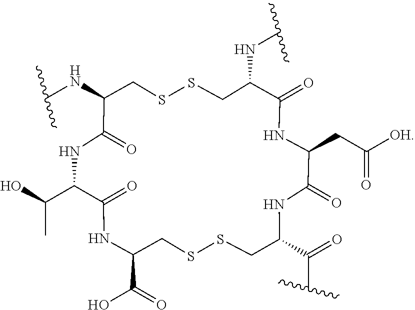 [SMILES: *N[C@H]1CSSC[C@H](N*)C(=O)N[C@@H]([C@@H](C)O)C(=O)N[C@H](C(=O)O)CSSC[C@@H](C(*)=O)NC(=O)[C@H](CC(=O)O)NC1=O]

In some embodiments, L¹ is

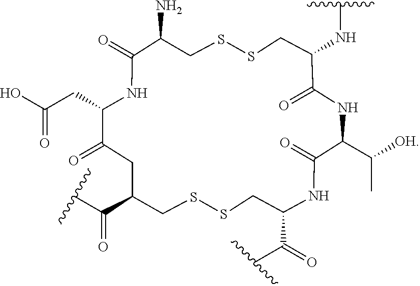 [SMILES: *N[C@H]1CSSC[C@H](N)C(=O)N[C@@H](CC(=O)O)C(=O)C[C@H](C(*)=O)CSSC[C@@H](C(*)=O)NC(=O)[C@H]([C@@H](C)O)NC1=O]

In some embodiments, L¹ is

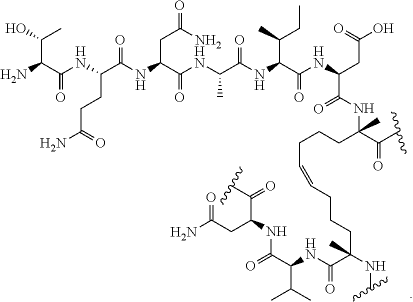 [SMILES: *N[C@@](C)(CCC/C=C\CCC[C@](C)(NC(=O)[C@H](CC(=O)O)NC(=O)[C@@H](NC(=O)[C@H](C)NC(=O)[C@H](CC(N)=O)NC(=O)[C@H](CCC(N)=O)NC(=O)[C@@H](N)[C@@H](C)O)[C@@H](C)CC)C(*)=O)C(=O)N[C@H](C(=O)N[C@@H](CC(N)=O)C(*)=O)C(C)C]

In some embodiments, L¹ is

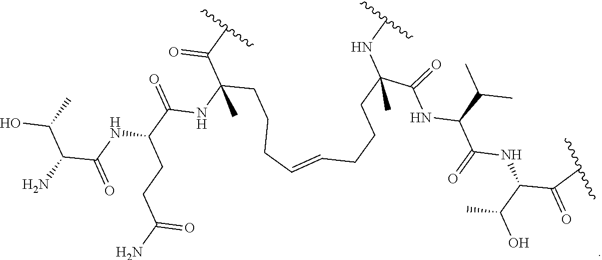 [SMILES: *N[C@@](C)(CCC/C=C/CCC[C@](C)(NC(=O)[C@H](CCC(N)=O)NC(=O)[C@H](N)[C@@H](C)O)C(*)=O)C(=O)N[C@H](C(=O)N[C@H](C(*)=O)[C@@H](C)O)C(C)C]

In some embodiments, L¹

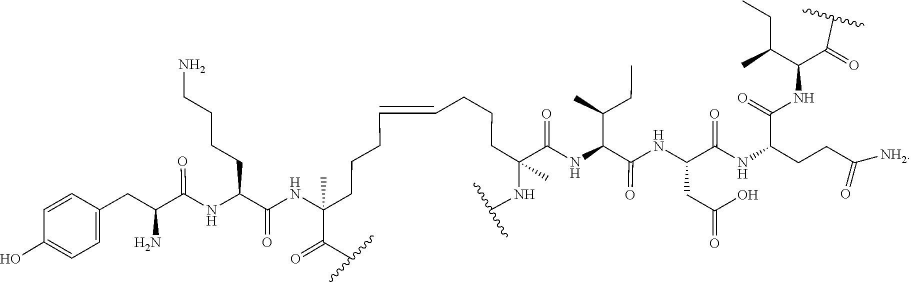 [SMILES: *N[C@@](C)(CCC/C=C/CCC[C@](C)(NC(=O)[C@H](CCCCN)NC(=O)[C@@H](N)Cc1ccc(O)cc1)C(*)=O)C(=O)N[C@H](C(=O)N[C@@H](CC(=O)O)C(=O)N[C@@H](CCC(N)=O)C(=O)N[C@H](C(*)=O)[C@@H](C)CC)[C@@H](C)CC]

In some embodiments, L¹ is

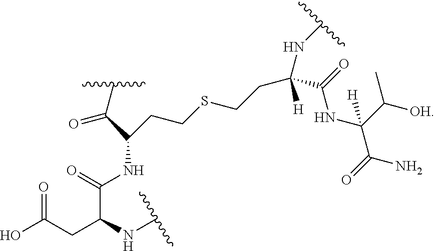

In some embodiments, L¹ is

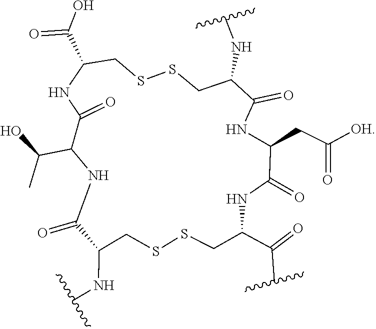 [SMILES: *N[C@H]1CSSC[C@@H](C(*)=O)NC(=O)[C@H](CC(=O)O)NC(=O)[C@@H](N*)CSSC[C@@H](C(=O)O)NC(=O)C([C@@H](C)O)NC1=O]

In some embodiments, L¹ is

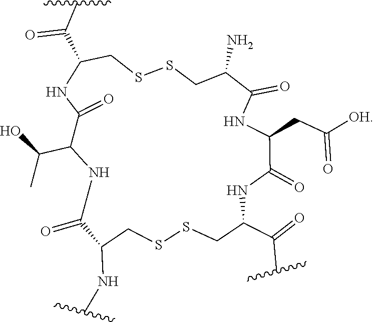 [SMILES: *N[C@H]1CSSC[C@@H](C(*)=O)NC(=O)[C@H](CC(=O)O)NC(=O)[C@@H](N)CSSC[C@@H](C(*)=O)NC(=O)C([C@@H](C)O)NC1=O]

In some embodiments, L¹ is

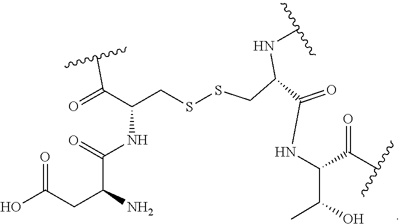 [SMILES: *N[C@@H](CSSC[C@H](NC(=O)[C@@H](N)CC(=O)O)C(*)=O)C(=O)N[C@H](C(*)=O)[C@@H](C)O]

In some embodiments, L¹ is selected from those depicted in Table 1, below.

As defined above and described herein, L² is a covalent bond or a C₁₋₁₀ bivalent straight or branched saturated or unsaturated hydrocarbon chain wherein 1-3 methylene units of the chain are independently and optionally replaced with —S—, —N(R)—, —O—, —C(O)—, —OC(O)—, —C(O)O—, —C(O)N(R)—, —N(R)C(O)—, —S(O)—, —S(O)₂—, —(CH₂OCH₂)n-, —(OCH₂CH₂)n-, or -Cy¹-, wherein each -Cy¹- is independently a 5-6 membered heteroarylenyl with 1-4 heteroatoms independently selected from nitrogen, oxygen or sulfur.

In some embodiments, L² is a covalent bond. In some embodiments, L² is a C₁₋₁₀ bivalent straight or branched saturated or unsaturated hydrocarbon chain wherein 1-3 methylene units of the chain are independently and optionally replaced with —S—, —N(R)—, —O—, —C(O)—, —OC(O)—, —C(O)O—, —C(O)N(R)—, —N(R)C(O)—, —S(O)—, —S(O)₂—, —(CH₂OCH₂)n-, —(OCH₂CH₂)n-, or -Cy¹-, wherein each -Cy¹- is independently a 5-6 membered heteroarylenyl with 1-4 heteroatoms independently selected from nitrogen, oxygen or sulfur.

In some embodiments, L² is

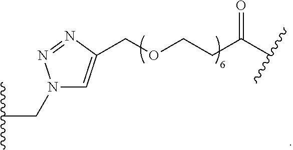

In some embodiments, L² is

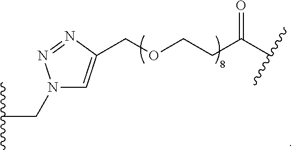

In some embodiments, L² is

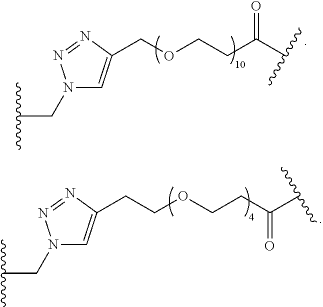

In some embodiments L² is

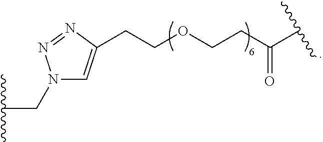

In some embodiments L² is

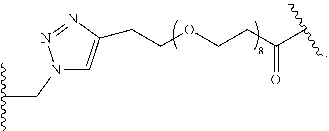

In some embodiments L² is

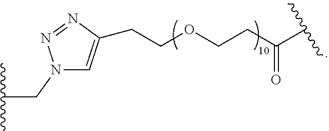

In some embodiments, L² is

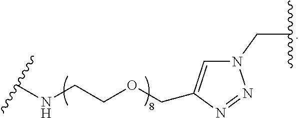

In some embodiments, L² is

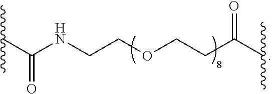

In some embodiments, L² is

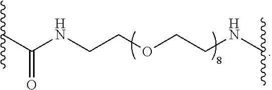

In some embodiments, L² is selected from those depicted in Table 1, below.

In some embodiments, L is L² as described in the present disclosure.

As defined above and described herein, TBT is a target binding moiety.

In some embodiments, TBT is a target binding moiety.

In some embodiments, TBT is

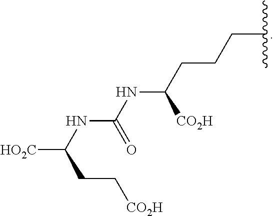 [SMILES: *CCC[C@H](NC(=O)N[C@@H](CCC(=O)O)C(=O)O)C(=O)O]

In some embodiments, TBT is

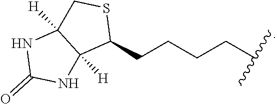 [SMILES: *CCCC[C@@H]1SC[C@@H]2NC(=O)N[C@@H]21]

In some embodiments, TBT is selected from those depicted in Table 1, below.

As defined above and described herein, each of m and n is independently 1, 2, 3, 4, 5, 6, 7, 8, 9, or 10.

In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10.

In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

As defined above and described herein, each of R⁷ is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; or: an R⁷ group and the R^{7′} group attached to the same carbon atom are optionally taken together with their intervening carbon atom to form a 3-8 membered saturated or partially unsaturated spirocyclic carbocyclic ring or a 4-8 membered saturated or partially unsaturated spirocyclic heterocyclic ring having 1-2 heteroatoms independently selected from nitrogen, oxygen, or sulfur.

In some embodiments, R⁷ is hydrogen. In some embodiments, R⁷ is optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur. In some embodiments, R⁷ is an optionally substituted C₁₋₆ aliphatic group. In some embodiments, R⁷ is an optionally substituted 3-8 membered saturated or partially unsaturated monocyclic carbocyclic ring. In some embodiments, R⁷ is an optionally substituted phenyl. In some embodiments, R⁷ is an optionally substituted 8-10 membered bicyclic aromatic carbocyclic ring. In some embodiments, R⁷ is an optionally substituted 4-8 membered saturated or partially unsaturated monocyclic heterocyclic ring having 1-2 heteroatoms independently selected from nitrogen, oxygen, or sulfur. In some embodiments, R⁷ is an optionally substituted 5-6 membered monocyclic heteroaromatic ring having 1-4 heteroatoms independently selected from nitrogen, oxygen, or sulfur. In some embodiments, R⁷ is an optionally substituted 8-10 membered bicyclic heteroaromatic ring having 1-5 heteroatoms independently selected from nitrogen, oxygen, or sulfur.

In some embodiments, R⁷ is methyl. In some embodiments, R⁷ is

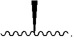

In some embodiments, R⁷ is

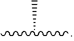

In some embodiments, R⁷ is

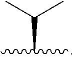

In some embodiments, R⁷ is

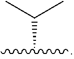

In some embodiments, R⁷ is

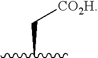

In some embodiments, R⁷ is

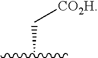

In some embodiments, R⁷ is

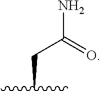

In some embodiments, R⁷ is

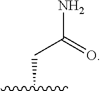

In some embodiments, R⁷ is

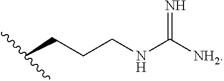

In some embodiments, R⁷ is

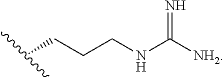

In some embodiments, R⁷ is

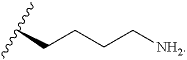

In some embodiments, R⁷ is

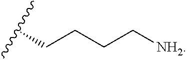

In some embodiments, R⁷ is

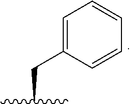

In some embodiments, R⁷ is

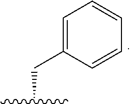

In some embodiments, R⁷ is

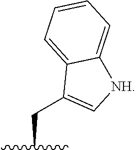

In some embodiments, R⁷ is

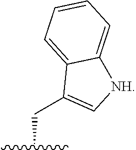

In some embodiments, R⁷ is

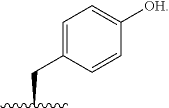

In some embodiments, R⁷ is

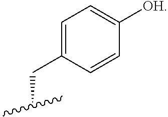

In some embodiments, R⁷ is

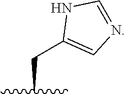

In some embodiments, R⁷ is

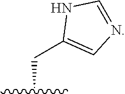

In some embodiments, an R⁷ group and the R^{7′} group attached to the same carbon atom are taken together with their intervening carbon atom to form a 3-8 membered saturated or partially unsaturated spirocyclic carbocyclic ring. In some embodiments, an R⁷ group and the R^{7′} group attached to the same carbon atom are taken together with their intervening carbon atom to form a 4-8 membered saturated or partially unsaturated spirocyclic heterocyclic ring having 1-2 heteroatoms independently selected from nitrogen, oxygen, or sulfur.

As defined above and described herein, each of R^{7′} is independently hydrogen or C₁₋₃ aliphatic.

In some embodiments, R^{7′} is hydrogen. In some embodiments, R^{7′} is methyl. In some embodiments, R^{7′} is ethyl. In some embodiments, R^{7′} is n-propyl. In some embodiments, R^{7′} is isopropyl.

As defined above and described herein, each of R⁸ is independently hydrogen, or C₁₋₄ aliphatic, or: an R⁸ group and its adjacent R⁷ group are optionally taken together with their intervening atoms to form a 4-8 membered saturated or partially unsaturated monocyclic heterocyclic ring having 1-2 heteroatoms independently selected from nitrogen, oxygen, or sulfur.

In some embodiments, R⁸ is hydrogen. In some embodiments, R⁸ is C₁₋₄ aliphatic. In some embodiments, R⁸ is methyl. In some embodiments, R⁸ is ethyl. In some embodiments, R⁸ is n-propyl. In some embodiments, R⁸ is isopropyl. In some embodiments, R⁸ is n-butyl. In some embodiments, R⁸ is isobutyl. In some embodiments, R⁸ is tert-butyl.

In some embodiments, an R⁸ group and its adjacent R⁷ group are taken together with their intervening atoms to form a 4-8 membered saturated or partially unsaturated monocyclic heterocyclic ring having 1-2 heteroatoms independently selected from nitrogen, oxygen, or sulfur.

In some embodiments, an R⁸ group and its adjacent R⁷ group are taken together with their intervening atoms to form

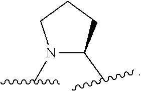 [SMILES: *[C@@H]1CCCN1*]

In some embodiments, an R⁸ group and its adjacent R⁷ group are taken together with their intervening atoms to form

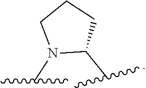 [SMILES: *[C@H]1CCCN1*]

As defined above and described herein, R⁹ is hydrogen, C₁₋₃ aliphatic, or —C(O)C₁₋₃ aliphatic.

In some embodiments, R⁹ is hydrogen. In some embodiments, R⁹ is C₁₋₃ aliphatic. In some embodiments, R⁹ is —C(O)C₁₋₃ aliphatic.

In some embodiments, R⁹ is methyl. In some embodiments, R⁹ is ethyl. In some embodiments, R⁹ is n-propyl. In some embodiments, R⁹ is isopropyl. In some embodiments, R⁹ is cyclopropyl.

In some embodiments, R⁹ is —C(O)Me. In some embodiments, R⁹ is —C(O)Et. In some embodiments, R⁹ is —C(O)CH₂CH₂CH₃. In some embodiments, R⁹ is —C(O)CH(CH₃)₂. In some embodiments, R⁹ is —C(O)cyclopropyl.

In some embodiments, R is R⁷ as described in the present disclosure. In some embodiments, R^{a2} is R⁷ as described in the present disclosure. In some embodiments, R³ is R^{7′} as described in the present disclosure. In some embodiments, R is R^{7′} as described in the present disclosure. In some embodiments, R^{a2} is R^{7′} as described in the present disclosure. In some embodiments, R³ is R^{7′} as described in the present disclosure. In some embodiments, R is R⁸ as described in the present disclosure. In some embodiments, R^{a2} is R⁸ as described in the present disclosure. In some embodiments, R^{a3} is R⁸ as described in the present disclosure. In some embodiments, R is R^{8′} as described in the present disclosure. In some embodiments, R^{a2} is R^{8′} as described in the present disclosure. In some embodiments, R^{a3} is R^{8′} as described in the present disclosure. In some embodiments, R is R⁹ as described in the present disclosure. In some embodiments, R^{a2} is R⁹ as described in the present disclosure. In some embodiments, R^{a3} is R⁹ as described in the present disclosure.

As defined above and described herein, L³ is a bivalent linker moiety that connects

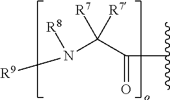

with TBT.

In some embodiments, L³ is a bivalent linker moiety that connects

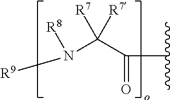

with TBT.

In some embodiments, L³ is

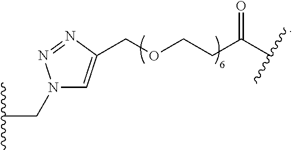

In some embodiments, L³ is

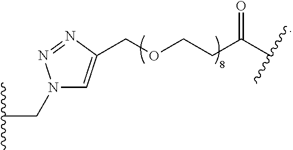

In some embodiments, L³ is

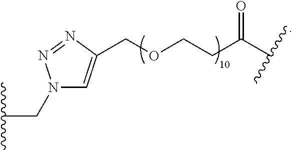

In some embodiments, L³ is

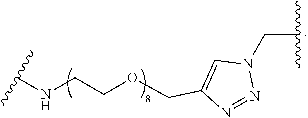

In some embodiments, L³ is

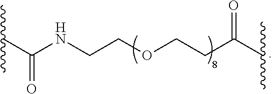

In some embodiments, L³ is

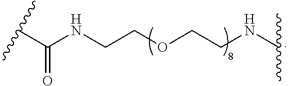

In some embodiments, L³ is selected from those depicted in Table 1, below.

In some embodiments, L is L³ as described in the present disclosure.

As defined above and described herein, o is 1, 2, 3, 4, 5, 6, 7, 8, 9, or 10.

In some embodiments, o is 1. In some embodiments, o is 2. In some embodiments, o is 3. In some embodiments, o is 4. In some embodiments, o is 5. In some embodiments, o is 6. In some embodiments, o is 7. In some embodiments, o is 8. In some embodiments, o is 9. In some embodiments, o is 10.

In some embodiments, R^{a1} is R as described in the present disclosure. In some embodiments, R^{a1} is optionally substituted C₁₋₄ aliphatic. In some embodiments, R^{a1} is optionally substituted C₁₋₄ alkyl. In some embodiments, R^{a1} is methyl.

In some embodiments, L^{a1} is L^{a} as described in the present disclosure. In some embodiments, L^{a1} is a covalent bond.

In some embodiments, L^{a2} is L^{a} as described in the present disclosure. In some embodiments, L^{a2} is a covalent bond.

In some embodiments, L^{T} is L^{a} as described herein. In some embodiments, L^{T} is L as described herein. In some embodiments, L^{T} is a covalent bond. In some embodiments, L^{T} is —CH₂—C(O)—. In some embodiments, L^{T} links a —S— of a side chain (e.g., through —CH₂) with the amino group of an amino acid residue (e.g., through —C(O)—).

In some embodiments, L^{a} is a covalent bond. In some embodiments, L^{a} is an optionally substituted bivalent group selected from C₁-C₁₀ aliphatic or C₁-C₁₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{a} is an optionally substituted bivalent group selected from C₁-C₅ aliphatic or C₁-C₅ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, -, -Cy-, —O—, —S—, —S—S—, —N(R′)—, —C(O)—, —C₂—, —S(O)₂N(R′)—, —C(O)S—, or —C(O)O—. In some embodiments, L^{a} is an optionally substituted bivalent C₁-C₅ aliphatic, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{a} is an optionally substituted bivalent C₁-C₅ aliphatic. In some embodiments, L^{a} is an optionally substituted bivalent C₁-C₅ heteroaliphatic having 1-3 heteroatoms independently selected from nitrogen, oxygen and sulfur.

In some embodiments, R^{a2} is R as described in the present disclosure. In some embodiments, R^{a2} is a side chain of a natural amino acid. In some embodiments, R^{a3} is R as described in the present disclosure. In some embodiments, R^{a3} is a side chain of a natural amino acid. In some embodiments, one of R^{2a} and R^{3a} is hydrogen. In some embodiments, R^{a2} and/or R^{a3} are R, wherein R is optionally substituted C₁₋₈ aliphatic or aryl. In some embodiments, R is optionally substituted linear C₂₋₈ alkyl. In some embodiments, R is linear C₂₋₈ alkyl. In some embodiments, R is optionally substituted branched C₂₋₈ alkyl. In some embodiments, R is branched C₂₋₈ alkyl. In some embodiments, R is n-pentyl. In some embodiments, R is optionally substituted phenyl. In some embodiments, R is optionally substituted —CH₂-phenyl. In some embodiments, R is 4-phenylphenyl-CH₂—.

In some embodiments, each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon. In some embodiments, each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon. In some embodiments, -Cy- is an optionally substituted ring as described in the present disclosure, for example, for R and Cy^{L}, but is bivalent.

In some embodiments, -Cy- is monocyclic. In some embodiments, -Cy- is bicyclic. In some embodiments, -Cy- is polycyclic. In some embodiments, -Cy- is saturated. In some embodiments, -Cy- is partially unsaturated. In some embodiments, -Cy- is aromatic. In some embodiments, -Cy- comprises a saturated monocyclic moiety. In some embodiments, -Cy- comprises a partially unsaturated monocyclic moiety. In some embodiments, -Cy- comprises an aromatic monocyclic moiety. In some embodiments, -Cy- comprises a combination of a saturated, a partially unsaturated, and/or an aromatic cyclic moiety. In some embodiments, -Cy- is or comprises 3-membered ring. In some embodiments, -Cy- is or comprises 4-membered ring. In some embodiments, -Cy- is or comprises 5-membered ring. In some embodiments, -Cy- is or comprises 6-membered ring. In some embodiments, -Cy- is or comprises 7-membered ring. In some embodiments, -Cy- is or comprises 8-membered ring. In some embodiments, -Cy- is or comprises 9-membered ring. In some embodiments, -Cy- is or comprises 10-membered ring. In some embodiments, -Cy- is or comprises 11-membered ring. In some embodiments, -Cy- is or comprises 12-membered ring. In some embodiments, -Cy- is or comprises 13-membered ring. In some embodiments, -Cy- is or comprises 14-membered ring. In some embodiments, -Cy- is or comprises 15-membered ring. In some embodiments, -Cy- is or comprises 16-membered ring. In some embodiments, -Cy- is or comprises 17-membered ring. In some embodiments, -Cy- is or comprises 18-membered ring. In some embodiments, -Cy- is or comprises 19-membered ring. In some embodiments, -Cy- is or comprises 20-membered ring.

In some embodiments, -Cy- is or comprises an optionally substituted bivalent C₃₋₂₀ cycloaliphatic ring. In some embodiments, -Cy- is or comprises an optionally substituted bivalent, saturated C₃₋₂₀ cycloaliphatic ring. In some embodiments, -Cy- is or comprises an optionally substituted bivalent, partially unsaturated C₃₋₂₀ cycloaliphatic ring. In some embodiments, -Cy-H is optionally substituted cycloaliphatic as described in the present disclosure, for example, cycloaliphatic embodiments for R.

In some embodiments, -Cy- is or comprises an optionally substituted C₆₋₂₀ aryl ring. In some embodiments, -Cy- is or comprises optionally substituted phenylene. In some embodiments, -Cy- is or comprises optionally substituted 1,2-phenylene. In some embodiments, -Cy- is or comprises optionally substituted 1,3-phenylene. In some embodiments, -Cy- is or comprises optionally substituted 1,4-phenylene. In some embodiments, -Cy- is or comprises an optionally substituted bivalent naphthalene ring. In some embodiments, -Cy-H is optionally substituted aryl as described in the present disclosure, for example, aryl embodiments for R.

In some embodiments, -Cy- is or comprises an optionally substituted bivalent 5-20 membered heteroaryl ring having 1-10 heteroatoms independently selected from oxygen, nitrogen, sulfur, phosphorus and silicon. In some embodiments, -Cy- is or comprises an optionally substituted bivalent 5-20 membered heteroaryl ring having 1-10 heteroatoms independently selected from oxygen, nitrogen, and sulfur. In some embodiments, -Cy- is or comprises an optionally substituted bivalent 5-6 membered heteroaryl ring having 1-4 heteroatoms independently selected from oxygen, nitrogen, sulfur. In some embodiments, -Cy- is or comprises an optionally substituted bivalent 5-6 membered heteroaryl ring having 1-3 heteroatoms independently selected from oxygen, nitrogen, sulfur. In some embodiments, -Cy- is or comprises an optionally substituted bivalent 5-6 membered heteroaryl ring having 1-2 heteroatoms independently selected from oxygen, nitrogen, sulfur. In some embodiments, -Cy- is or comprises an optionally substituted bivalent 5-6 membered heteroaryl ring having one heteroatom independently selected from oxygen, nitrogen, sulfur. In some embodiments, -Cy-H is optionally substituted heteroaryl as described in the present disclosure, for example, heteroaryl embodiments for R. In some embodiments, -Cy- is

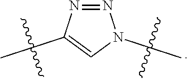 [SMILES: *c1cn(*)nn1]

In some embodiments, -Cy- is or comprises an optically substituted bivalent 3-20 membered heterocyclyl ring having 1-10 heteroatoms independently selected from oxygen, nitrogen, sulfur, phosphorus and silicon. In some embodiments, -Cy- is or comprises an optionally substituted bivalent 3-20 membered heterocyclyl ring having 1-10 heteroatoms independently selected from oxygen, nitrogen, and sulfur. In some embodiments, -Cy- is or comprises an optionally substituted bivalent 3-6 membered heterocyclyl ring having 1-4 heteroatoms independently selected from oxygen, nitrogen, sulfur. In some embodiments, -Cy- is or comprises an optionally substituted bivalent 5-6 membered heterocyclyl ring having 1-4 heteroatoms independently selected from oxygen, nitrogen, sulfur. In some embodiments, -Cy- is or comprises an optionally substituted bivalent 5-6 membered heterocyclyl ring having 1-3 heteroatoms independently selected from oxygen, nitrogen, sulfur. In some embodiments, -Cy- is or comprises an optionally substituted bivalent 5-6 membered heterocyclyl ring having 1-2 heteroatoms independently selected from oxygen, nitrogen, sulfur. In some embodiments, -Cy- is or comprises an optionally substituted bivalent 5-6 membered heterocyclyl ring having one heteroatom independently selected from oxygen, nitrogen, sulfur. In some embodiments, -Cy- is or comprises an optionally substituted saturated bivalent heterocyclyl group. In some embodiments, -Cy- is or comprises an optionally substituted partially unsaturated bivalent heterocyclyl group. In some embodiments, -Cy-H is optionally substituted heterocyclyl as described in the present disclosure, for example, heterocyclyl embodiments for R.

In some embodiments, -Cy- is

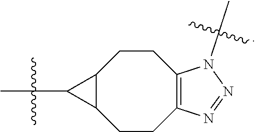 [SMILES: *C1C2CCc3nnn(*)c3CCC12]

In some embodiments, -Cy- is

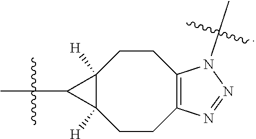 [SMILES: *C1[C@H]2CCc3c(nnn3*)CC[C@@H]12]

In some embodiments, -Cy- is

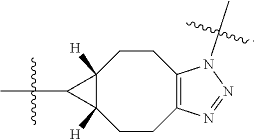 [SMILES: *C1[C@H]2CCc3nnn(*)c3CC[C@@H]12]

In some embodiments, -Cy- is

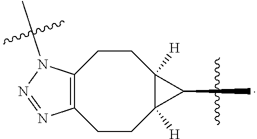 [SMILES: *n1nnc2c1CC[C@H]1[C@@H](C)[C@H]1CC2]

In some embodiments, -Cy- is

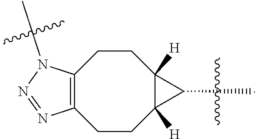 [SMILES: *n1nnc2c1CC[C@@H]1[C@H](C)[C@@H]1CC2]

In some embodiments, each Xaa is independently an amino acid residue. In some embodiments, each Xaa is independently an amino acid residue of an amino acid of formula A-I.

In some embodiments, t is 0. In some embodiments, t is 1-50. In some embodiments, t is z as described in the present disclosure.

In some embodiments, y is 1. In some embodiments, y is 2. In some embodiments, y is 3. In some embodiments, y is 4. In some embodiments, y is 5. In some embodiments, y is 6. In some embodiments, y is 7. In some embodiments, y is 8. In some embodiments, y is 9. In some embodiments, y is 10. In some embodiments, y is 11. In some embodiments, y is 12. In some embodiments, y is 13. In some embodiments, y is 14. In some embodiments, y is 15. In some embodiments, y is 16. In some embodiments, y is 17. In some embodiments, y is 18. In some embodiments, y is 19. In some embodiments, y is 20. In some embodiments, y is greater than 20.

In some embodiments, z is 1. In some embodiments, z is 2. In some embodiments, z is 3. In some embodiments, z is 4. In some embodiments, z is 5. In some embodiments, z is 6. In some embodiments, z is 7. In some embodiments, z is 8. In some embodiments, z is 9. In some embodiments, z is 10. In some embodiments, z is 11. In some embodiments, z is 12. In some embodiments, z is 13. In some embodiments, z is 14. In some embodiments, z is 15. In some embodiments, z is 16. In some embodiments, z is 17. In some embodiments, z is 18. In some embodiments, z is 19. In some embodiments, z is 20. In some embodiments, z is greater than 20.

In some embodiments, R^{c} is R′ as described in the present disclosure. In some embodiments, R^{c} is R as described in the present disclosure. In some embodiments, R^{c} is —N(R′)₂, wherein each R′ is independently as described in the present disclosure. In some embodiments, R^{c} is —NH₂. In some embodiments, R^{c} is R—C(O)—, wherein R is as described in the present disclosure. In some embodiments, R^{c} is —H.

In some embodiments, a is 1. In some embodiments, a is 1-100, 2-100, 1-50, 1-20, or 1-10. In some embodiments, a is 5. In some embodiments, a is 10. In some embodiments, a is 20. In some embodiments, a is 50.

In some embodiments, b is 1. In some embodiments, b is 1-100, 2-100, 1-50, 1-20, or 1-10. In some embodiments, b is 5. In some embodiments, b is 10. In some embodiments, b is 20. In some embodiments, b is 50.

In some embodiments, a1 is 0. In some embodiments, a1 is 1.

In some embodiments, a2 is 0. In some embodiments, a2 is 1.

In some embodiments, L^{b} is L^{a} as described in the present disclosure. In some embodiments, L^{b} comprises -Cy-. In some embodiments, L^{b} comprises a double bond. In some embodiments, L^{b} comprises —S—. In some embodiments, L^{b} comprises —S—S—. In some embodiments, L^{b} comprises —C(O)—N(R′)—.

In some embodiments, R′ is —R, —C(O)R, —C(O)OR, or —S(O)₂R, wherein R is as described in the present disclosure. In some embodiments, R′ is R, wherein R is as described in the present disclosure. In some embodiments, R′ is —C(O)R, wherein R is as described in the present disclosure. In some embodiments, R′ is —C(O)OR, wherein R is as described in the present disclosure. In some embodiments, R′ is —S(O)₂R, wherein R is as described in the present disclosure. In some embodiments, R′ is hydrogen. In some embodiments, R′ is not hydrogen. In some embodiments, R′ is R, wherein R is optionally substituted C₁₋₂₀ aliphatic as described in the present disclosure. In some embodiments, R′ is R, wherein R is optionally substituted C₁₋₂₀ heteroaliphatic as described in the present disclosure. In some embodiments, R′ is R, wherein R is optionally substituted C₆₋₂₀ aryl as described in the present disclosure. In some embodiments, R′ is R, wherein R is optionally substituted C₆₋₂₀ arylaliphatic as described in the present disclosure. In some embodiments, R′ is R, wherein R is optionally substituted C₆₋₂₀ arylheteroaliphatic as described in the present disclosure. In some embodiments, R′ is R, wherein R is optionally substituted 5-20 membered heteroaryl as described in the present disclosure. In some embodiments, R′ is R, wherein R is optionally substituted 3-20 membered heterocyclyl as described in the present disclosure. In some embodiments, two or more R′ are R, and are optionally and independently taken together to form an optionally substituted ring as described in the present disclosure.

In some embodiments, each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon.
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, each R is independently —H, or an optionally substituted group selected from C₁₋₂₀ aliphatic, C₁₋₂₀ heteroaliphatic having 1-10 heteroatoms independently selected from oxygen, nitrogen, sulfur, phosphorus and silicon, C₆₋₂₀ aryl, C₆₋₂₀ arylaliphatic, C₆₋₂₀ arylheteroaliphatic having 1-10 heteroatoms independently selected from oxygen, nitrogen, sulfur, phosphorus and silicon, 5-20 membered heteroaryl having 1-10 heteroatoms independently selected from oxygen, nitrogen, sulfur, phosphorus and silicon, and 3-20 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-20 membered monocyclic, bicyclic or polycyclic ring having, in addition to the atom, 0-10 heteroatoms independently selected from oxygen, nitrogen, sulfur, phosphorus and silicon.
two or more R groups on two or more atoms are optionally and independently taken together with their intervening atoms to form an optionally substituted, 3-20 membered monocyclic, bicyclic or polycyclic ring having, in addition to the intervening atoms, 0-10 heteroatoms independently selected from oxygen, nitrogen, sulfur, phosphorus and silicon.

In some embodiments, each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon.

In some embodiments, each R is independently —H, or an optionally substituted group selected from C₁₋₂₀ aliphatic, C₁₋₂₀ heteroaliphatic having 1-10 heteroatoms independently selected from oxygen, nitrogen, sulfur, phosphorus and silicon, C₆₋₂₀ aryl, C₆₋₂₀ arylaliphatic, C₆₋₂₀ arylheteroaliphatic having 1-10 heteroatoms independently selected from oxygen, nitrogen, sulfur, phosphorus and silicon, 5-20 membered heteroaryl having 1-10 heteroatoms independently selected from oxygen, nitrogen, sulfur, phosphorus and silicon, and 3-20 membered heterocyclyl having 1-10 heteroatoms independently selected from oxygen, nitrogen, sulfur, phosphorus and silicon.

In some embodiments, R is hydrogen. In some embodiments, R is not hydrogen. In some embodiments, R is an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, a 5-30 membered heteroaryl ring having 1-10 heteroatoms independently selected from oxygen, nitrogen, sulfur, phosphorus and silicon, and a 3-30 membered heterocyclic ring having 1-10 heteroatoms independently selected from oxygen, nitrogen, sulfur, phosphorus and silicon.

In some embodiments, R is hydrogen or an optionally substituted group selected from C₁₋₂₀ aliphatic, phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, and sulfur, a 4-7 membered saturated or partially unsaturated heterocyclic ring having 1-3 heteroatoms independently selected from nitrogen, oxygen, and sulfur, a 7-10 membered bicyclic saturated or partially unsaturated heterocyclic ring having 1-5 heteroatoms independently selected from nitrogen, oxygen, and sulfur, or an 8-10 membered bicyclic heteroaryl ring having 1-5 heteroatoms independently selected from nitrogen, oxygen, and sulfur.

In some embodiments, R is optionally substituted C₁₋₃₀ aliphatic. In some embodiments, R is optionally substituted C₁₋₂₀ aliphatic. In some embodiments, R is optionally substituted C₁₋₁₅ aliphatic. In some embodiments, R is optionally substituted C₁₋₁₀ aliphatic. In some embodiments, R is optionally substituted C₁₋₆ aliphatic. In some embodiments, R is optionally substituted C₁₋₆ alkyl. In some embodiments, R is optionally substituted hexyl, pentyl, butyl, propyl, ethyl or methyl. In some embodiments, R is optionally substituted hexyl. In some embodiments, R is optionally substituted pentyl. In some embodiments, R is optionally substituted butyl. In some embodiments, R is optionally substituted propyl. In some embodiments, R is optionally substituted ethyl. In some embodiments, R is optionally substituted methyl. In some embodiments, R is hexyl. In some embodiments, R is pentyl. In some embodiments, R is butyl. In some embodiments, R is propyl. In some embodiments, R is ethyl. In some embodiments, R is methyl. In some embodiments, R is isopropyl. In some embodiments, R is n-propyl. In some embodiments, R is tert-butyl. In some embodiments, R is sec-butyl. In some embodiments, R is n-butyl. In some embodiments, R is —(CH₂)₂CN.

In some embodiments, R is optionally substituted C₃₋₃₀ cycloaliphatic. In some embodiments, R is optionally substituted C₃₋₂₀ cycloaliphatic. In some embodiments, R is optionally substituted C₃₋₁₀ cycloaliphatic. In some embodiments, R is optionally substituted cyclohexyl. In some embodiments, R is cyclohexyl. In some embodiments, R is optionally substituted cyclopentyl. In some embodiments, R is cyclopentyl. In some embodiments, R is optionally substituted cyclobutyl. In some embodiments, R is cyclobutyl. In some embodiments, R is optionally substituted cyclopropyl. In some embodiments, R is cyclopropyl.

In some embodiments, R is an optionally substituted 3-30 membered saturated or partially unsaturated carbocyclic ring. In some embodiments, R is an optionally substituted 3-7 membered saturated or partially unsaturated carbocyclic ring. In some embodiments, R is an optionally substituted 3-membered saturated or partially unsaturated carbocyclic ring. In some embodiments, R is an optionally substituted 4-membered saturated or partially unsaturated carbocyclic ring. In some embodiments, R is an optionally substituted 5-membered saturated or partially unsaturated carbocyclic ring. In some embodiments, R is an optionally substituted 6-membered saturated or partially unsaturated carbocyclic ring. In some embodiments, R is an optionally substituted 7-membered saturated or partially unsaturated carbocyclic ring. In some embodiments, R is optionally substituted cycloheptyl. In some embodiments, R is cycloheptyl. In some embodiments, R is optionally substituted cyclohexyl. In some embodiments, R is cyclohexyl. In some embodiments, R is optionally substituted cyclopentyl. In some embodiments, R is cyclopentyl. In some embodiments, R is optionally substituted cyclobutyl. In some embodiments, R is cyclobutyl. In some embodiments, R is optionally substituted cyclopropyl. In some embodiments, R is cyclopropyl.

In some embodiments, when R is or comprises a ring structure, e.g., cycloaliphatic, cycloheteroaliphatic, aryl, heteroaryl, etc., the ring structure can be monocyclic, bicyclic or polycyclic. In some embodiments, R is or comprises a monocyclic structure. In some embodiments, R is or comprises a bicyclic structure. In some embodiments, R is or comprises a polycyclic structure.

In some embodiments, R is optionally substituted C₁₋₃₀ heteroaliphatic having 1-10 heteroatoms independently selected from oxygen, nitrogen, sulfur, phosphorus and silicon. In some embodiments, R is optionally substituted C₁₋₂₀ heteroaliphatic having 1-10 heteroatoms. In some embodiments, R is optionally substituted C₁₋₂₀ heteroaliphatic having 1-10 heteroatoms independently selected from oxygen, nitrogen, sulfur, phosphorus or silicon, optionally including one or more oxidized forms of nitrogen, sulfur, phosphorus or selenium. In some embodiments R is optionally substituted C₁₋₃₀ heteroaliphatic comprising 1-10 groups independently selected from

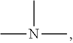

### —N═, ≡N, —S—, —S(O)—, —S(O)₂—, —O—, ═O,

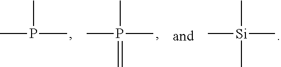

In some embodiments, R is optionally substituted C₆₋₃₀ aryl. In some embodiments, R is optionally substituted phenyl. In some embodiments, R is phenyl. In some embodiments, R is substituted phenyl.

In some embodiments, R is an optionally substituted 8-10 membered bicyclic saturated, partially unsaturated or aryl ring. In some embodiments, R is an optionally substituted 8-10 membered bicyclic saturated ring. In some embodiments, R is an optionally substituted 8-10 membered bicyclic partially unsaturated ring. In some embodiments, R is an optionally substituted 8-10 membered bicyclic aryl ring. In some embodiments, R is optionally substituted naphthyl.

In some embodiments, R is optionally substituted 5-30 membered heteroaryl ring having 1-10 heteroatoms independently selected from oxygen, nitrogen, sulfur, phosphorus and silicon. In some embodiments, R is optionally substituted 5-30 membered heteroaryl ring having 1-10 heteroatoms independently selected from oxygen, nitrogen, and sulfur. In some embodiments, R is optionally substituted 5-30 membered heteroaryl ring having 1-5 heteroatoms independently selected from oxygen, nitrogen, sulfur, phosphorus and silicon. In some embodiments, R is optionally substituted 5-30 membered heteroaryl ring having 1-5 heteroatoms independently selected from oxygen, nitrogen, and sulfur.

In some embodiments, R is an optionally substituted 5-6 membered monocyclic heteroaryl ring having 1-4 heteroatoms independently selected from nitrogen, oxygen, and sulfur. In some embodiments, R is a substituted 5-6 membered monocyclic heteroaryl ring having 1-4 heteroatoms independently selected from nitrogen, oxygen, and sulfur. In some embodiments, R is an unsubstituted 5-6 membered monocyclic heteroaryl ring having 1-4 heteroatoms independently selected from nitrogen, oxygen, and sulfur. In some embodiments, R is an optionally substituted 5-6 membered monocyclic heteroaryl ring having 1-3 heteroatoms independently selected from nitrogen, sulfur, and oxygen. In some embodiments, R is a substituted 5-6 membered monocyclic heteroaryl ring having 1-3 heteroatoms independently selected from nitrogen, oxygen, and sulfur. In some embodiments, R is an unsubstituted 5-6 membered monocyclic heteroaryl ring having 1-3 heteroatoms independently selected from nitrogen, sulfur, and oxygen.

In some embodiments, R is an optionally substituted 5-membered monocyclic heteroaryl ring having 1-4 heteroatoms independently selected from nitrogen, oxygen or sulfur. In some embodiments, R is an optionally substituted 6-membered monocyclic heteroaryl ring having 1-4 heteroatoms independently selected from nitrogen, oxygen, and sulfur.

In some embodiments, R is an optionally substituted 5-membered monocyclic heteroaryl ring having one heteroatom selected from nitrogen, oxygen, and sulfur. In some embodiments, R is optionally substituted pyrrolyl, furanyl, or thienyl.

In some embodiments, R is an optionally substituted 5-membered heteroaryl ring having two heteroatoms independently selected from nitrogen, oxygen, and sulfur. In certain embodiments, R is an optionally substituted 5-membered heteroaryl ring having one nitrogen atom, and an additional heteroatom selected from sulfur or oxygen. In some embodiments, R is an optionally substituted 5-membered heteroaryl ring having three heteroatoms independently selected from nitrogen, oxygen, and sulfur. In some embodiments, R is an optionally substituted 5-membered heteroaryl ring having four heteroatoms independently selected from nitrogen, oxygen, and sulfur.

In some embodiments, R is an optionally substituted 6-membered heteroaryl ring having 1-4 nitrogen atoms. In some embodiments, R is an optionally substituted 6-membered heteroaryl ring having 1-3 nitrogen atoms. In other embodiments, R is an optionally substituted 6-membered heteroaryl ring having 1-2 nitrogen atoms. In some embodiments, R is an optionally substituted 6-membered heteroaryl ring having four nitrogen atoms. In some embodiments, R is an optionally substituted 6-membered heteroaryl ring having three nitrogen atoms. In some embodiments, R is an optionally substituted 6-membered heteroaryl ring having two nitrogen atoms. In certain embodiments, R is an optionally substituted 6-membered heteroaryl ring having one nitrogen atom.

In certain embodiments, R is an optionally substituted 8-10 membered bicyclic heteroaryl ring having 1-4 heteroatoms independently selected from nitrogen, oxygen, and sulfur. In some embodiments, R is an optionally substituted 5,6-fused heteroaryl ring having 1-4 heteroatoms independently selected from nitrogen, oxygen, and sulfur. In certain embodiments, R is an optionally substituted 6,6-fused heteroaryl ring having 1-4 heteroatoms independently selected from nitrogen, oxygen, and sulfur.

In some embodiments, R is 3-30 membered heterocyclic ring having 1-10 heteroatoms independently selected from oxygen, nitrogen, sulfur, phosphorus and silicon. In some embodiments, R is 3-30 membered heterocyclic ring having 1-10 heteroatoms independently selected from oxygen, nitrogen, and sulfur. In some embodiments, R is 3-30 membered heterocyclic ring having 1-5 heteroatoms independently selected from oxygen, nitrogen, sulfur, phosphorus and silicon. In some embodiments, R is 3-30 membered heterocyclic ring having 1-5 heteroatoms independently selected from oxygen, nitrogen, and sulfur.

In some embodiments, R is an optionally substituted 3-7 membered saturated or partially unsaturated heterocyclic ring having 1-3 heteroatoms independently selected from nitrogen, oxygen, and sulfur. In some embodiments, R is a substituted 3-7 membered saturated or partially unsaturated heterocyclic ring having 1-3 heteroatoms independently selected from nitrogen, oxygen, and sulfur. In some embodiments, R is an unsubstituted 3-7 membered saturated or partially unsaturated heterocyclic ring having 1-3 heteroatoms independently selected from nitrogen, oxygen, and sulfur. In certain embodiments, R is an optionally substituted 5-7 membered partially unsaturated monocyclic ring having 1-3 heteroatoms independently selected from nitrogen, oxygen, and sulfur. In certain embodiments, R is an optionally substituted 5-6 membered partially unsaturated monocyclic ring having 1-3 heteroatoms independently selected from nitrogen, oxygen, and sulfur. In certain embodiments, R is an optionally substituted 5-membered partially unsaturated monocyclic ring having 1-3 heteroatoms independently selected from nitrogen, oxygen, and sulfur. In certain embodiments, R is an optionally substituted 6-membered partially unsaturated monocyclic ring having 1-3 heteroatoms independently selected from nitrogen, oxygen, and sulfur. In certain embodiments, R is an optionally substituted 7-membered partially unsaturated monocyclic ring having 1-3 heteroatoms independently selected from nitrogen, oxygen, and sulfur. In some embodiments, R is optionally substituted 3-membered heterocyclic ring having one heteroatom selected from nitrogen, oxygen or sulfur. In some embodiments, R is optionally substituted 4-membered heterocyclic ring having 1-3 heteroatoms independently selected from nitrogen, oxygen, and sulfur. In some embodiments, R is optionally substituted 5-membered heterocyclic ring having 1-3 heteroatoms independently selected from nitrogen, oxygen, and sulfur. In some embodiments, R is optionally substituted 6-membered heterocyclic ring having 1-3 heteroatoms independently selected from nitrogen, oxygen, and sulfur. In some embodiments, R is optionally substituted 7-membered heterocyclic ring having 1-3 heteroatoms independently selected from nitrogen, oxygen, and sulfur.

In some embodiments, R is an optionally substituted 3-membered saturated or partially unsaturated heterocyclic ring having 1-2 heteroatoms independently selected from nitrogen, oxygen, and sulfur. In some embodiments, R is an optionally substituted 4-membered saturated or partially unsaturated heterocyclic ring having 1-2 heteroatoms independently selected from nitrogen, oxygen, and sulfur. In some embodiments, R is an optionally substituted 5-membered saturated or partially unsaturated heterocyclic ring having 1-2 heteroatoms independently selected from nitrogen, oxygen, and sulfur. In some embodiments, R is an optionally substituted 6-membered saturated or partially unsaturated heterocyclic ring having 1-2 heteroatoms independently selected from nitrogen, oxygen, and sulfur. In some embodiments, R is an optionally substituted 7-membered saturated or partially unsaturated heterocyclic ring having 1-2 heteroatoms independently selected from nitrogen, oxygen, and sulfur.

In certain embodiments, R is an optionally substituted 5-6 membered partially unsaturated monocyclic ring having 1-2 heteroatoms independently selected from nitrogen, oxygen, and sulfur. In certain embodiments, R is an optionally substituted tetrahydropyridinyl, dihydrothiazolyl, dihydrooxazolyl, or oxazolinyl group.

In some embodiments, R is an optionally substituted 7-10 membered bicyclic saturated or partially unsaturated heterocyclic ring having 1-5 heteroatoms independently selected from nitrogen, oxygen, and sulfur. In some embodiments, R is optionally substituted indolinyl. In some embodiments, R is optionally substituted isoindolinyl. In some embodiments, R is optionally substituted 1, 2, 3, 4-tetrahydroquinolinyl. In some embodiments, R is optionally substituted 1, 2, 3, 4-tetrahydroisoquinolinyl. In some embodiments, R is an optionally substituted azabicyclo[3.2.1]octanyl.

In some embodiments, R is an optionally substituted 8-10 membered bicyclic heteroaryl ring having 1-5 heteroatoms independently selected from nitrogen, oxygen, and sulfur. In some embodiments, R is an optionally substituted 5,6-fused heteroaryl ring having 1-5 heteroatoms independently selected from nitrogen, oxygen, and sulfur.

In some embodiments, R is optionally substituted C₆₋₃₀ arylaliphatic. In some embodiments, R is optionally substituted C₆₋₂₀ arylaliphatic. In some embodiments, R is optionally substituted C₆₋₁₀ arylaliphatic. In some embodiments, an aryl moiety of the arylaliphatic has 6, 10, or 14 aryl carbon atoms. In some embodiments, an aryl moiety of the arylaliphatic has 6 aryl carbon atoms. In some embodiments, an aryl moiety of the arylaliphatic has 10 aryl carbon atoms. In some embodiments, an aryl moiety of the arylaliphatic has 14 aryl carbon atoms. In some embodiments, an aryl moiety is optionally substituted phenyl.

In some embodiments, R is optionally substituted C₆₋₃₀ arylheteroaliphatic having 1-10 heteroatoms independently selected from oxygen, nitrogen, sulfur, phosphorus and silicon. In some embodiments, R is optionally substituted C₆₋₃₀ arylheteroaliphatic having 1-10 heteroatoms independently selected from oxygen, nitrogen, and sulfur. In some embodiments, R is optionally substituted C₆₋₂₀ arylheteroaliphatic having 1-10 heteroatoms independently selected from oxygen, nitrogen, sulfur, phosphorus and silicon. In some embodiments, R is optionally substituted C₆₋₂₀ arylheteroaliphatic having 1-10 heteroatoms independently selected from oxygen, nitrogen, and sulfur. In some embodiments, R is optionally substituted C₆₋₁₀ arylheteroaliphatic having 1-5 heteroatoms independently selected from oxygen, nitrogen, sulfur, phosphorus and silicon. In some embodiments, R is optionally substituted C₆₋₁₀ arylheteroaliphatic having 1-5 heteroatoms independently selected from oxygen, nitrogen, and sulfur.

In some embodiments, two R groups are optionally and independently taken together to form a covalent bond. In some embodiments, —C═O is formed. In some embodiments, —C═C— is formed. In some embodiments, —C≡C— is formed.

In some embodiments, two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon. In some embodiments, two or more R groups on the same atom are optionally and independently taken together with the atom to form an optionally substituted, 3-20 membered monocyclic, bicyclic or polycyclic ring having, in addition to the atom, 0-10 heteroatoms independently selected from oxygen, nitrogen, sulfur, phosphorus and silicon. In some embodiments, two or more R groups on the same atom are optionally and independently taken together with the atom to form an optionally substituted, 3-10 membered monocyclic, bicyclic or polycyclic ring having, in addition to the atom, 0-5 heteroatoms independently selected from oxygen, nitrogen, sulfur, phosphorus and silicon. In some embodiments, two or more R groups on the same atom are optionally and independently taken together with the atom to form an optionally substituted, 3-6 membered monocyclic, bicyclic or polycyclic ring having, in addition to the atom, 0-3 heteroatoms independently selected from oxygen, nitrogen, sulfur, phosphorus and silicon. In some embodiments, two or more R groups on the same atom are optionally and independently taken together with the atom to form an optionally substituted, 3-5 membered monocyclic, bicyclic or polycyclic ring having, in addition to the atom, 0-3 heteroatoms independently selected from oxygen, nitrogen, sulfur, phosphorus and silicon.

In some embodiments, two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon. In some embodiments, two or more R groups on two or more atoms are optionally and independently taken together with their intervening atoms to form an optionally substituted, 3-20 membered monocyclic, bicyclic or polycyclic ring having, in addition to the intervening atoms, 0-10 heteroatoms independently selected from oxygen, nitrogen, sulfur, phosphorus and silicon. In some embodiments, two or more R groups on two or more atoms are optionally and independently taken together with their intervening atoms to form an optionally substituted, 3-10 membered monocyclic, bicyclic or polycyclic ring having, in addition to the intervening atoms, 0-10 heteroatoms independently selected from oxygen, nitrogen, sulfur, phosphorus and silicon. In some embodiments, two or more R groups on two or more atoms are optionally and independently taken together with their intervening atoms to form an optionally substituted, 3-10 membered monocyclic, bicyclic or polycyclic ring having, in addition to the intervening atoms, 0-5 heteroatoms independently selected from oxygen, nitrogen, sulfur, phosphorus and silicon. In some embodiments, two or more R groups on two or more atoms are optionally and independently taken together with their intervening atoms to form an optionally substituted, 3-6 membered monocyclic, bicyclic or polycyclic ring having, in addition to the intervening atoms, 0-3 heteroatoms independently selected from oxygen, nitrogen, sulfur, phosphorus and silicon. In some embodiments, two or more R groups on two or more atoms are optionally and independently taken together with their intervening atoms to form an optionally substituted, 3-5 membered monocyclic, bicyclic or polycyclic ring having, in addition to the intervening atoms, 0-3 heteroatoms independently selected from oxygen, nitrogen, sulfur, phosphorus and silicon.

In some embodiments, heteroatoms in R groups, or in the structures formed by two or more R groups taken together, are selected from oxygen, nitrogen, and sulfur. In some embodiments, a formed ring is 3, 4, 5, 6, 7, 8, 9, 10, 11, 12, 13, 14, 15, 16, 17, 18, 19, or 20-membered. In some embodiments, a formed ring is saturated. In some embodiments, a formed ring is partially saturated. In some embodiments, a formed ring is aromatic. In some embodiments, a formed ring comprises a saturated, partially saturated, or aromatic ring moiety. In some embodiments, a formed ring comprises 5, 6, 7, 8, 9, 10, 11, 12, 13, 14, 15, 16, 17, 18, 19, or 20 aromatic ring atoms. In some embodiments, a formed contains no more than 5, 6, 7, 8, 9, 10, 11, 12, 13, 14, 15, 16, 17, 18, 19, or 20 aromatic ring atoms. In some embodiments, aromatic ring atoms are selected from carbon, nitrogen, oxygen and sulfur.

In some embodiments, a ring formed by two or more R groups (or two or more groups selected from R and variables that can be R) taken together is a C₃₋₃₀ cycloaliphatic, C₆₋₃₀ aryl, 5-30 membered heteroaryl having 1-10 heteroatoms independently selected from oxygen, nitrogen, sulfur, phosphorus and silicon, or 3-30 membered heterocyclyl having 1-10 heteroatoms independently selected from oxygen, nitrogen, sulfur, phosphorus and silicon, ring as described for R, but bivalent or multivalent.

Exemplified compounds are set forth in Table 1, below. In some embodiments, an agent is or comprise a compound selected from Table 1 or a salt, e.g. pharmaceutically acceptable salt, thereof.

TABLE 1
Exemplified compounds, agents, and compositions.
Ac-DKEEILNKIYEIMRLLDELGNAEASMRVSDLILEFMKKGDERLLEEAERLLEEVER-NH₂
(SEQ ID NO: 91)
I-1

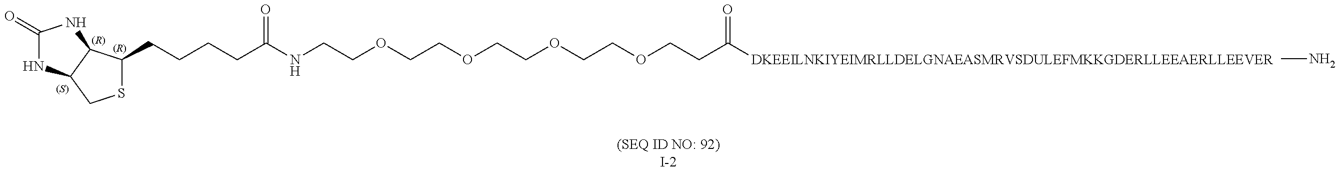

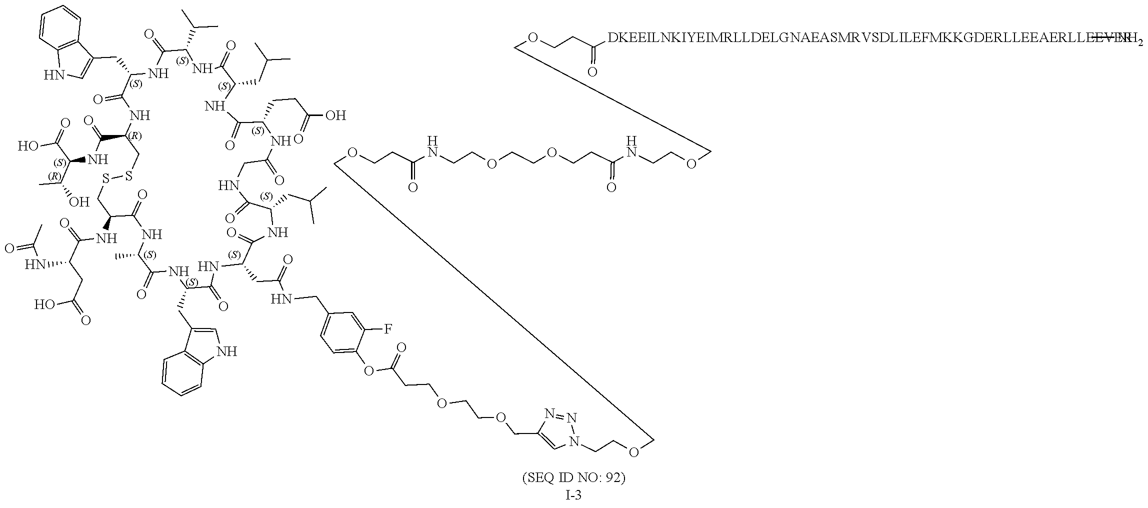

DKEWILQKIYEIMRLLDELGHAEASMRVSDLIYEFMKKGDERLLEEAERLLEEVER
(SEQ ID NO: 3)
I-4
Ac-DKEWILQKIYEIMRLLDELGHAEASMRVSDLIYEFMKKGDERLLEEAERLLEEVER
(SEQ ID NO: 93)
I-5

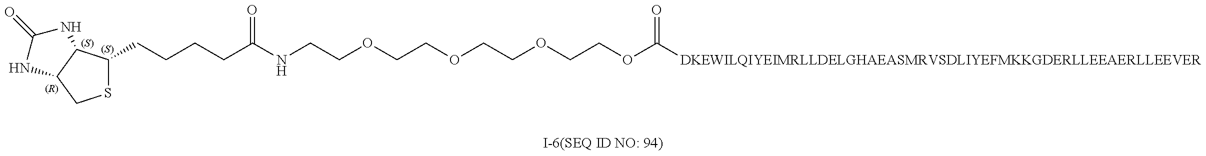

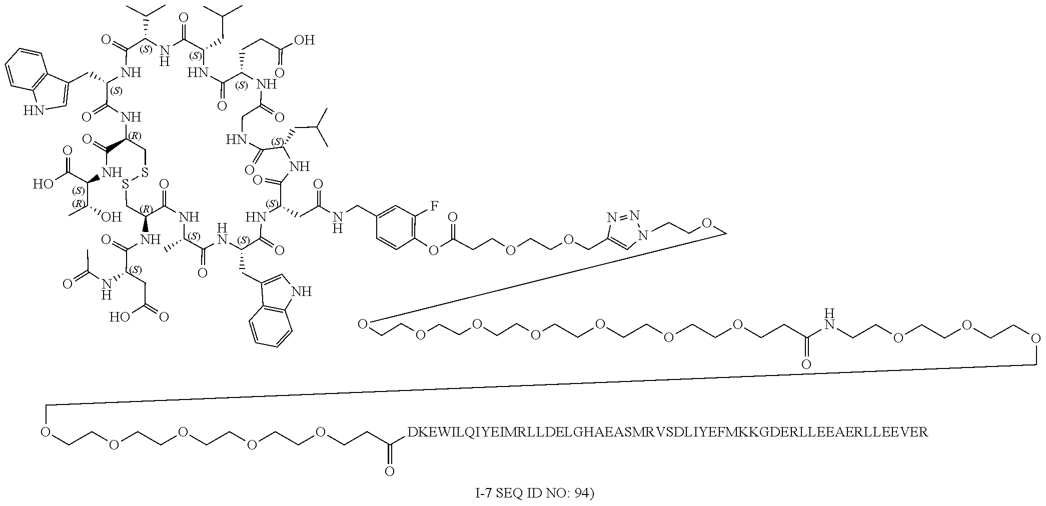

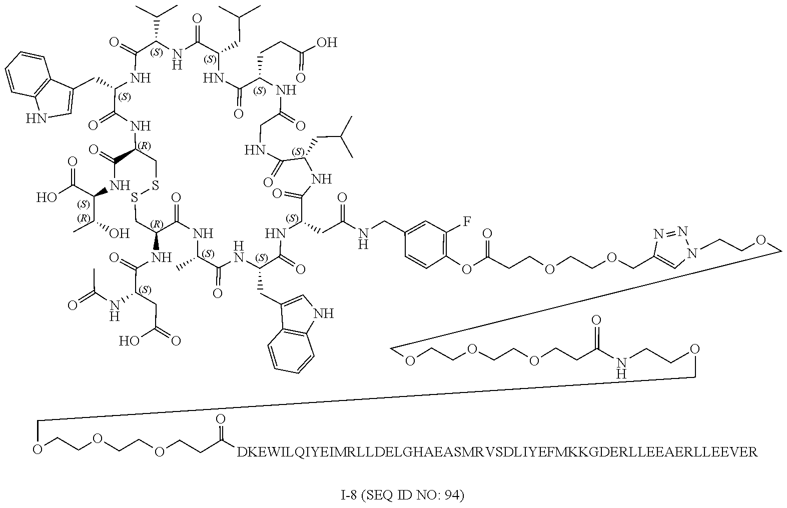

NDDELHMLMTDLVYEALHFAKDEEIKKRVFQLFELADKAYKNNDRQKLEKVVEELKELLERLL
S (SEQ ID NO: 6)
I-9
Ac-
NDDELHMLMTDLVYEALHFAKDEEIKKRVFQLFELADKAYKNNDRQKLEKVVEELKELLERLLS
(SEQ ID NO: 95)
I-10

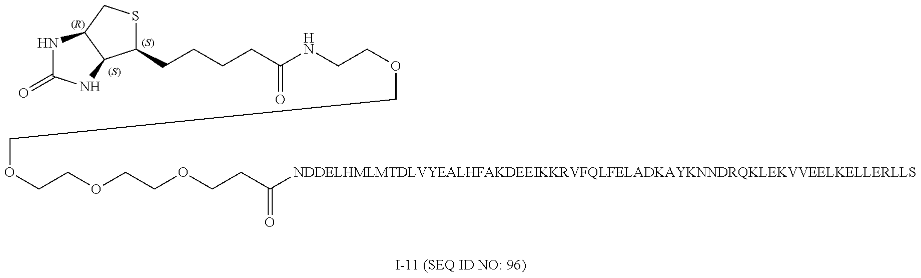

DKEWILQKIYEIMRLLDELGHAEASMRVSDLIYEFMKKGDERLLEEAERLLEEVER-NH2
I-12 (SEQ ID NO: 97)
Ac-DKEWILQKIYEIMRLLDELGHAEASMRVSDLIYEFMKKGDERLLEEAERLLEEVER-NH2
I-13 (SEQ ID NO: 98)

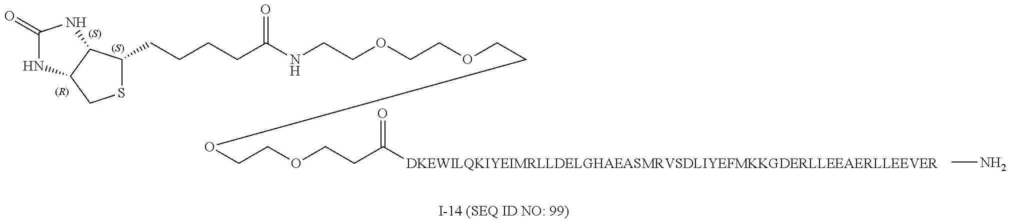

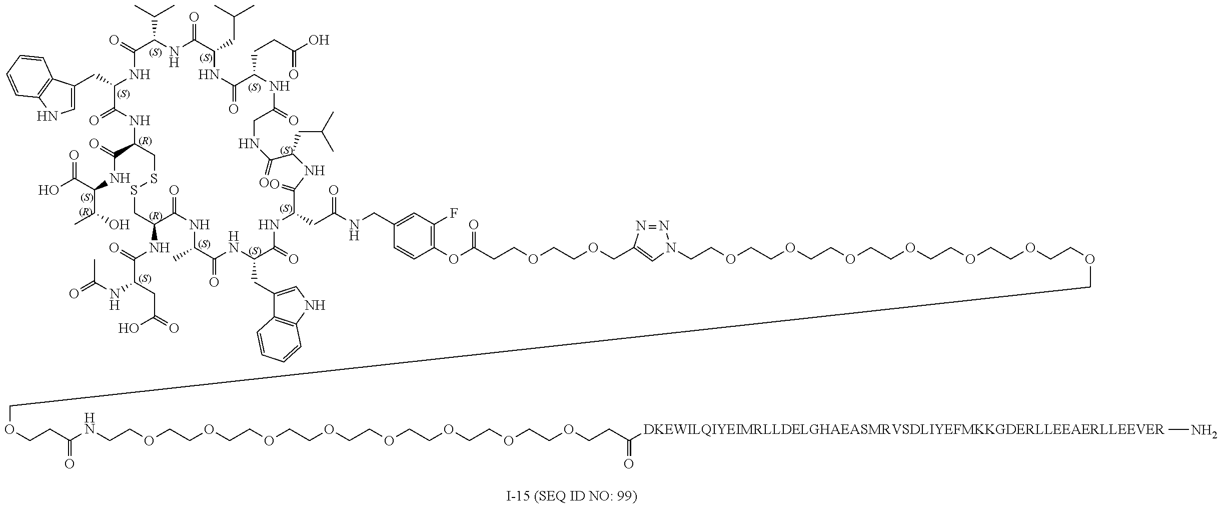

NDDELHMLMTDLVYEALHFAKDEEIKKRVFQLFELADKAYKNNDRQKLEKVVEELKELLERLLS-NH₂
I-16 (SEQ ID NO: 100)
Ac-NDDELHMLMTDLVYEALHFAKDEEIKKRVFQLFELADKAYKNNDRQKLEKVVEELKELLERLLS-NH₂
I-17 (SEQ ID NO: 101)

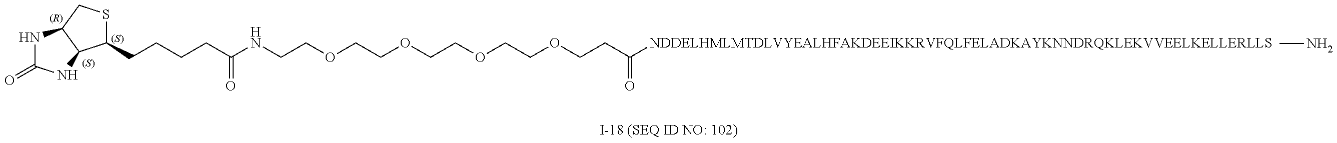

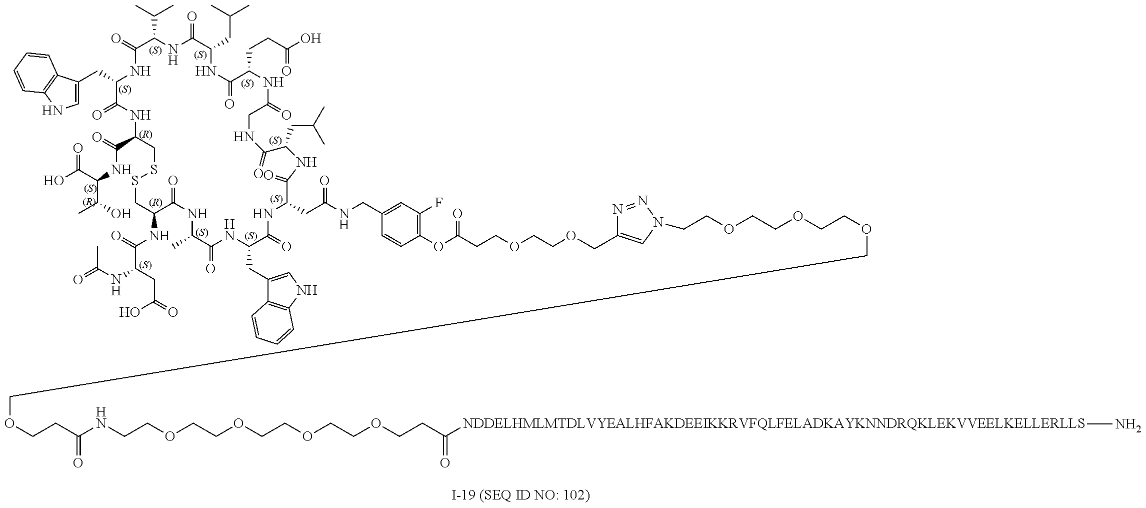

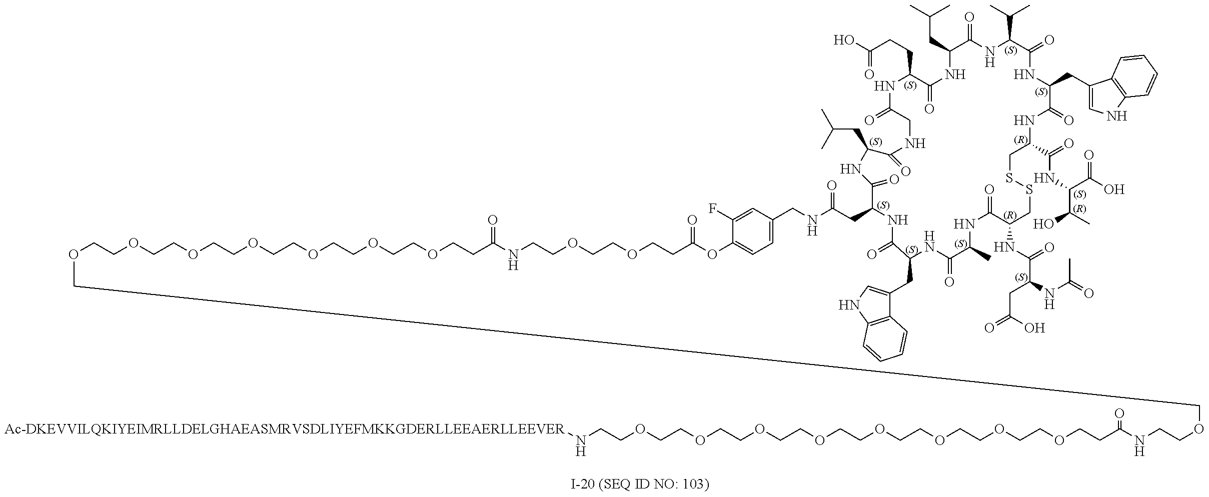

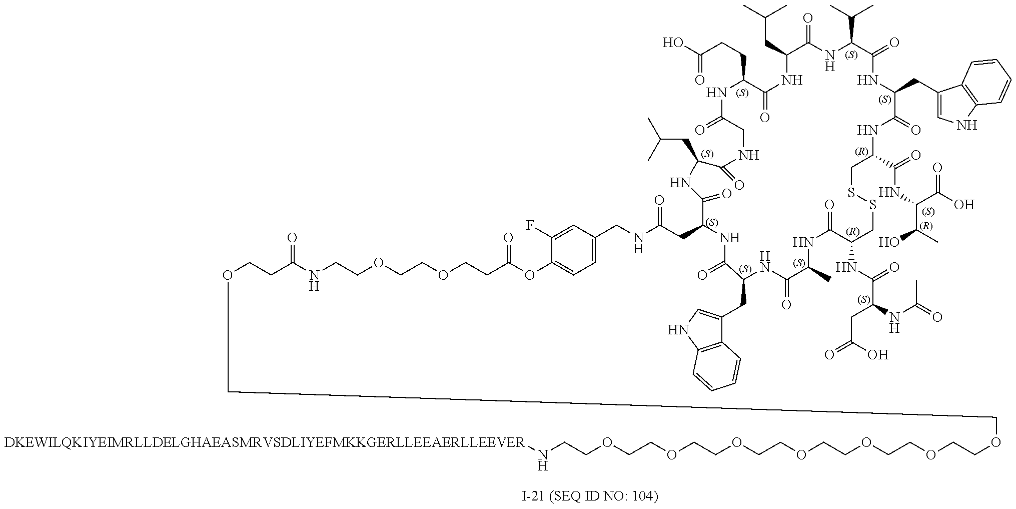

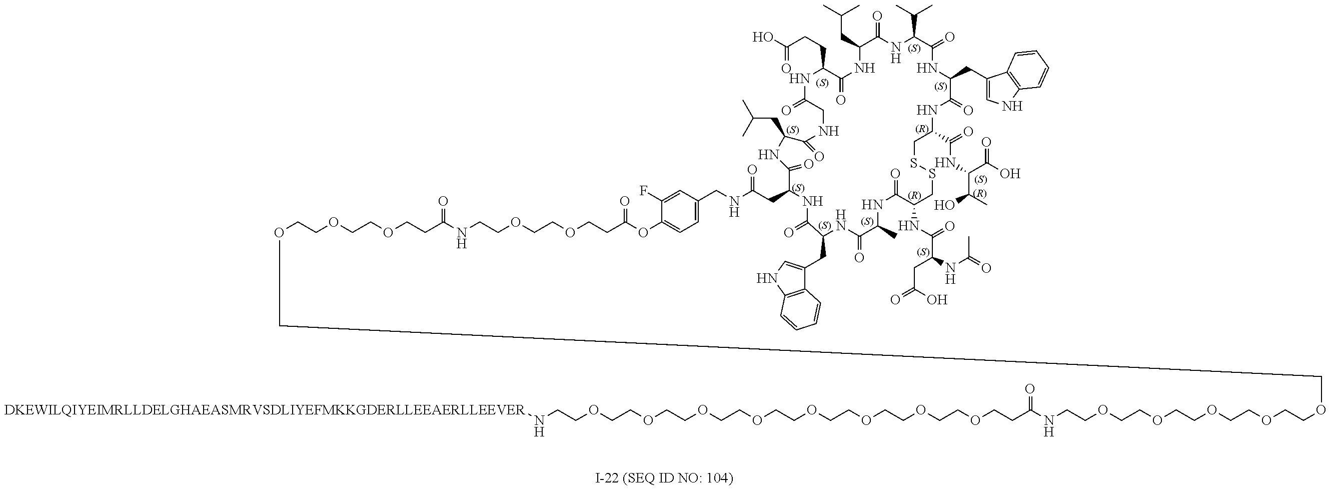

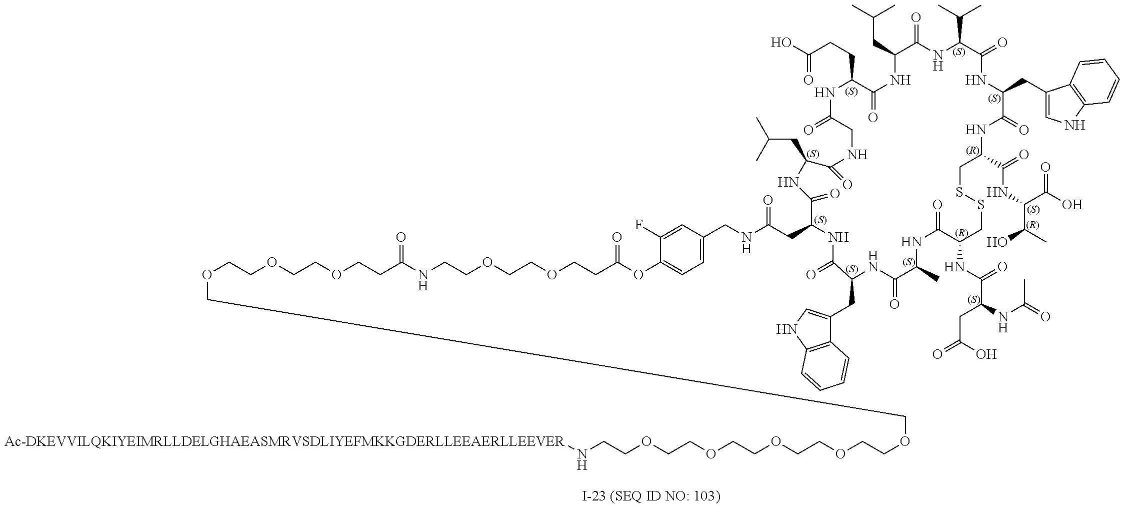

DKEEILNKIYEIMRLLDELGNAEASMRVSDLILEFMKKGDERLLEEAERLLEEVERggs EQKLISE
EDLGSGGGGSLPETGGggsggs SHHHHHHHHHH (SEQ ID NO: 105)
I-24
NDDELLMLVTDLVAEALLFAKDEEIKKRVFTLFELADKAYKNNDRDTLSKVVSELKELLERLQS
ggsEQKLISEEDLGSGGGGSLPETGGggsggs SHHHHHHHHHH (SEQ ID NO: 106)
I-25

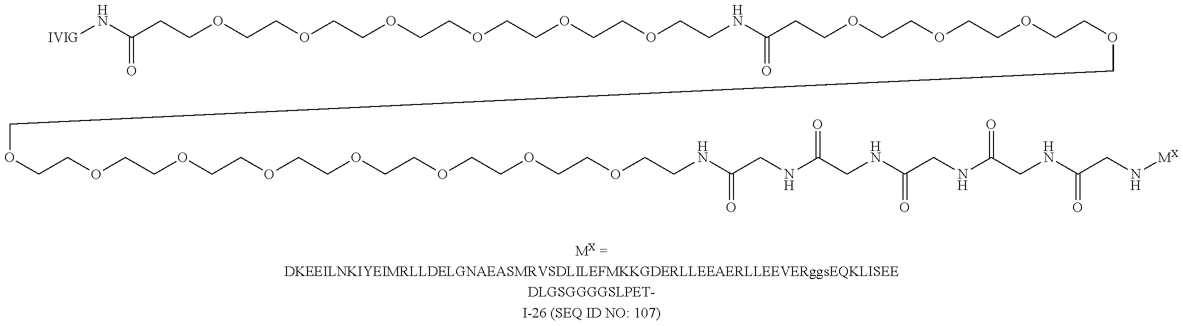

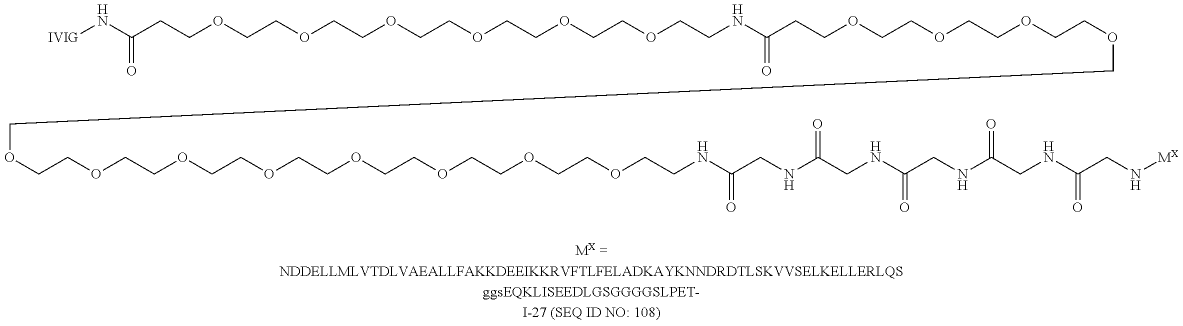

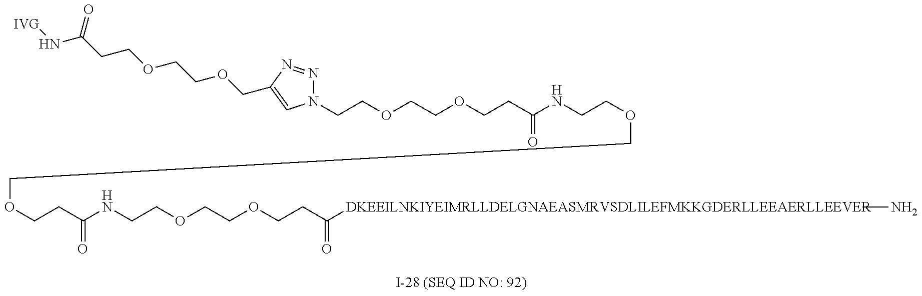

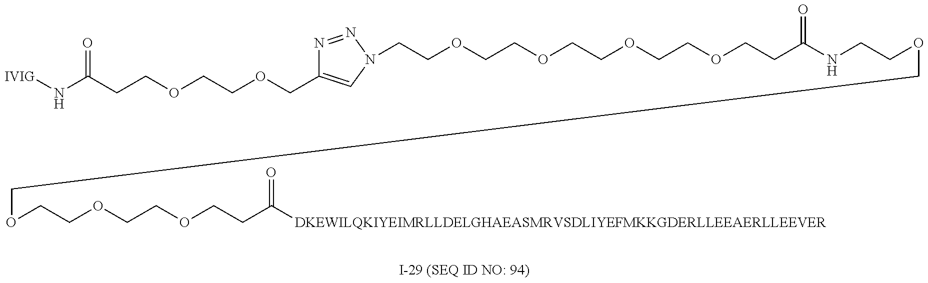

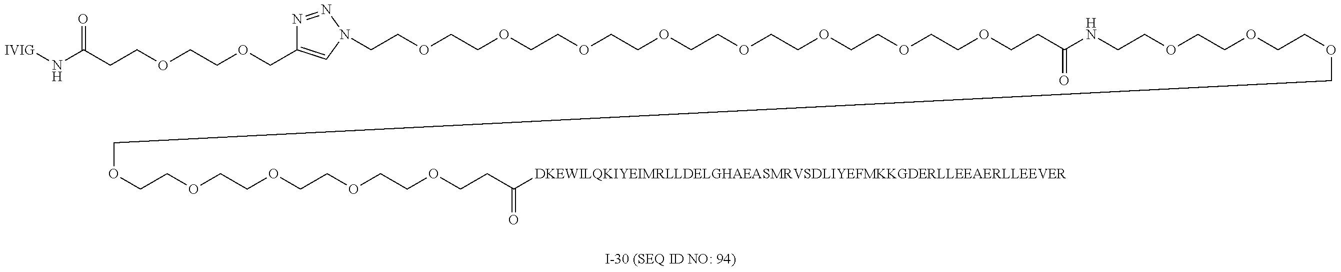

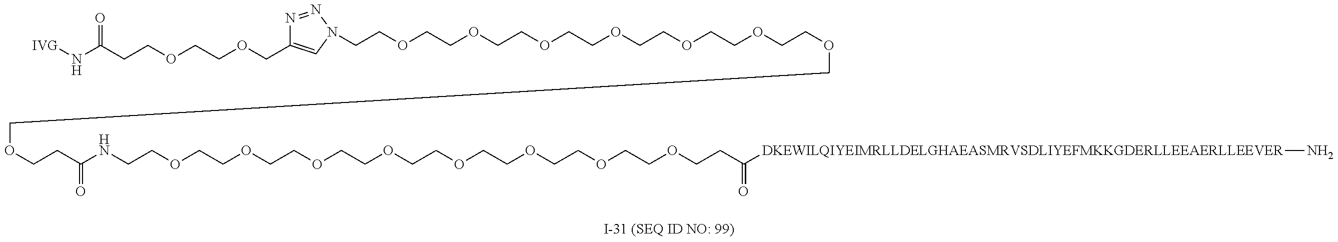

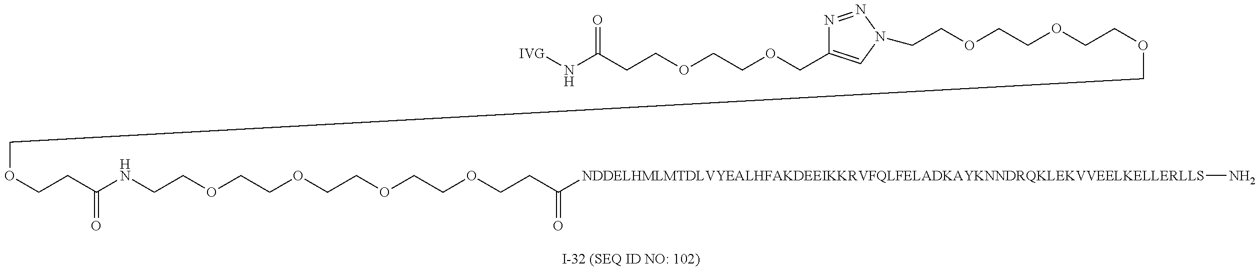

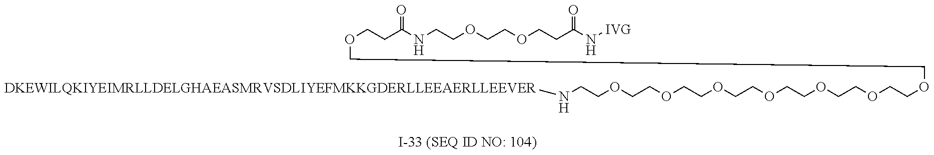

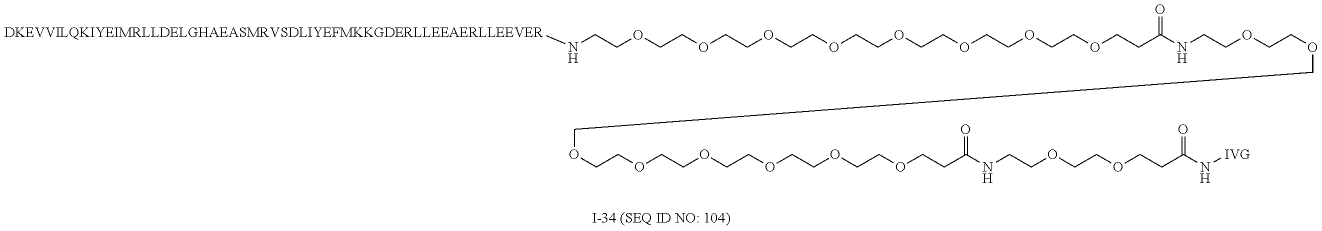

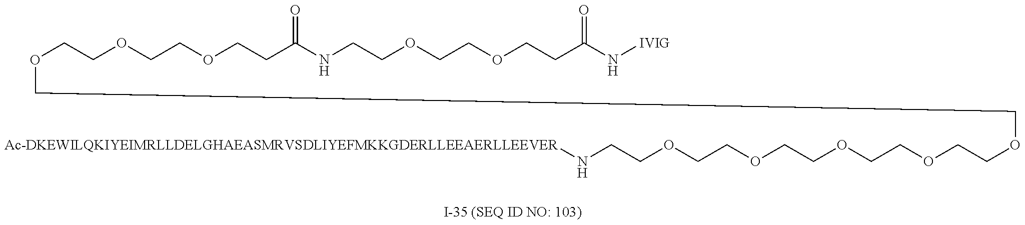

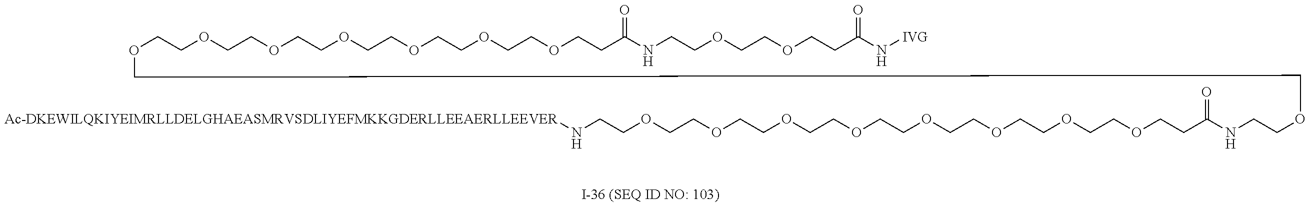

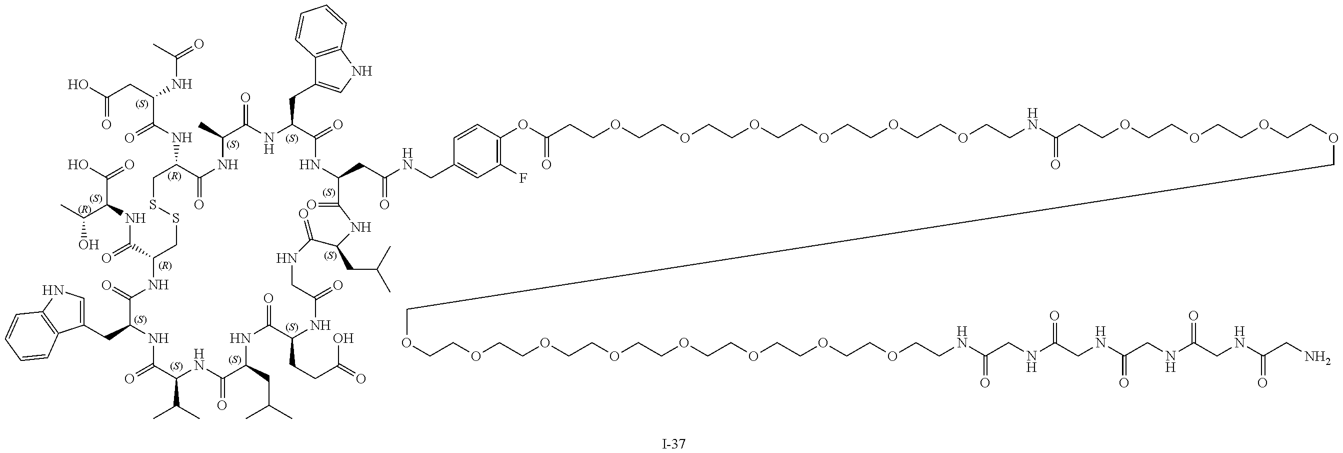

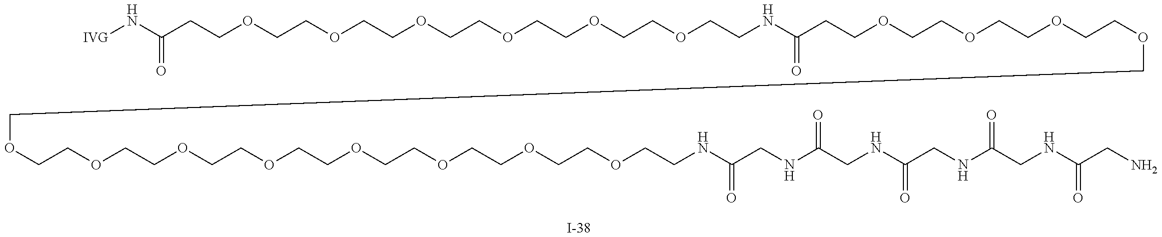

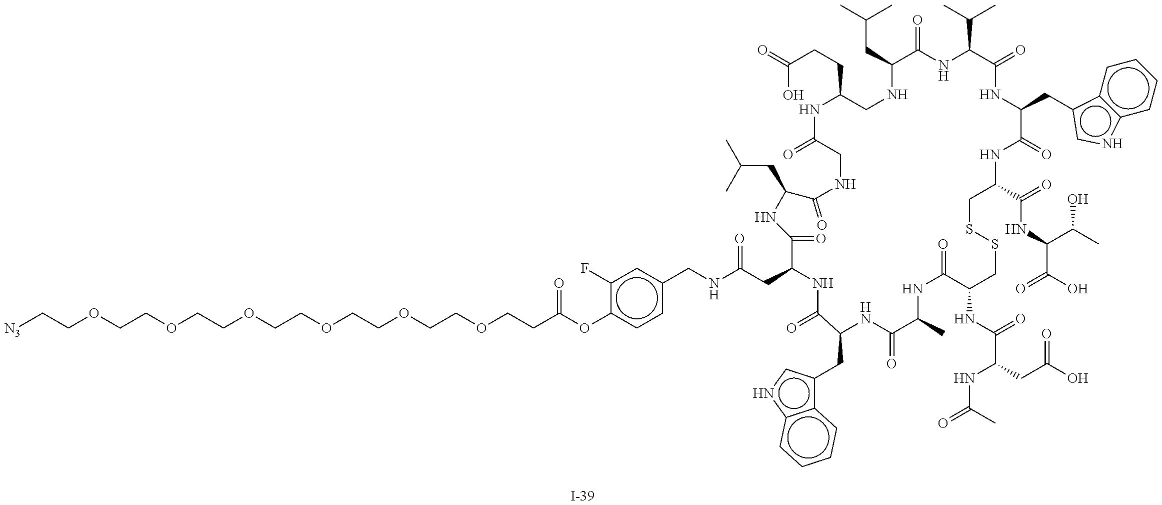

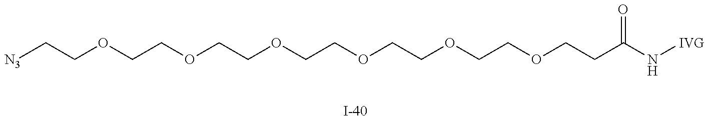

In various embodiments, for agents comprising IVIG, conjugation is through lysine side chains at K246 and/or K248 of an IgG1 heavy chain and amino acid residues corresponding thereto, K251 and/or K253 of an IgG2 heavy chain and amino acid residues corresponding thereto, and/or K239 and/or K241 of an IgG4 heavy chain and amino acid residues corresponding thereto. In some embodiments, IVIG is Gamunex-C. In some embodiments, IVIG is Flebogamma. In some embodiments, IgG1, IgG2, and/or IgG4 antibody moieties are from Gamunex-C. In some embodiments, IgG1, IgG2, and/or IgG4 antibody moieties are from Flebogamma. In some embodiments, for antibody moieties comprising two heavy chains, both chains are independently conjugated at one or more locations as described herein.

In some embodiments, the present disclosure provides a compound set forth in Table 1, above, or a pharmaceutically acceptable salt thereof.

In some embodiments, provided agents are conjugates of antibodies (e.g., IgG of a subject, pooled IgG, IVIG, etc.) with moieties comprising -(Xaa)y- (e.g., target binding moieties, agents of formula T-1, etc.) optionally through linker moieties (e.g., L). In some embodiments, provided agents are IVIG conjugates with target binding moieties optionally through linker moieties. In some embodiments, the present disclosure provides a plurality of such agents. In some embodiments, the present disclosure provides compositions comprising such agents. In some embodiments, the present disclosure provides compositions comprising a plurality of such agents.

In some embodiments, an antibody moiety is or comprises an IgG moiety (or a fragment thereof). In some embodiments, an antibody moiety is IVIG.

In some embodiments, the present disclosure provides a composition comprising a plurality of agents, wherein each agent independently comprises:
an antibody binding moiety,
a target binding moiety, and
optionally a linker moiety linking an antibody binding moiety and a target binding moiety.

In some embodiments, the present disclosure provides compositions comprising a plurality of such agents.

In some embodiments, each agent of the plurality is independently an agent described herein. In some embodiments, one or more agents of a plurality share the same target binding moiety. In some embodiments, agents of a plurality share the same target moiety. In some embodiments, one or more agents of a plurality share the same linker moiety. In some embodiments, agents of a plurality share the same linker moiety. In some embodiments, agents of a plurality recruit immune cells.

In some embodiments, one or more agents of a plurality each independently comprise an IgG moiety. In some embodiments, one or more agents of a plurality each independently comprise an IgG1 moiety. In some embodiments, one or more agents of a plurality each independently comprise an IgG2 moiety. In some embodiments, antibody moieties of one or more agents of a plurality each independently are or comprise IgG1 or a fragment thereof. In some embodiments, antibody moieties of one or more agents of a plurality each independently are or comprise IgG2 or a fragment thereof. In some embodiments, antibody moieties of one or more agents of a plurality each independently are or comprise IgG4 or a fragment thereof. In some embodiments, antibody moieties of agents of a plurality each independently are or comprise IgG1, IgG2, or IgG4, or a fragment thereof. In some embodiments, antibody moieties of agents of a plurality are independently of one or more antibodies in IVIG. In some embodiments, antibody moieties are of IgG1, IgG2 and/or IgG4 antibodies or fragments thereof in IVIG. In some embodiments, a plurality of agents is enriched (e.g., percentage) for certain antibody subclasses, e.g., IgG1, IgG2 and/or IgG4, compared to a reference composition. In some embodiments, a composition is enriched (e.g., percentage) for certain antibody subclasses, e.g., IgG1, IgG2 and/or IgG4, compared to a reference composition. In some embodiments, IgG1 is enriched. In some embodiments, IgG2 is enriched. In some embodiments, IgG3 is enriched. In some embodiments, IgG4 is enriched. In some embodiments, IgG1, IgG2 and IgG4 is enriched. In some embodiments, a reference composition is an IVIG composition.

In some embodiments, a fragment is or comprise a Fc region or a fragment thereof.

In some embodiments, one or more agents of a plurality can each independently bind to an Fc receptor. In some embodiments, one or more agents of a plurality can each independently interact hFcγRIIIA. In some embodiments, one or more agents of a plurality can each independently interact hFcγRIIIA on macrophages. In some embodiments, one or more agents of a plurality each independently comprise an antibody moiety that can interact hFcγRIIIA. In some embodiments, one or more agents of a plurality each independently comprise an antibody moiety that can interact hFcγRIIIA on macrophages. In some embodiments, one or more agents of a plurality can each independently interact hFcγRIIA. In some embodiments, one or more agents of a plurality can each independently interact hFcγRIIA on dendritic cells. In some embodiments, one or more agents of a plurality each independently comprise an antibody moiety that can interact hFcγRIIA. In some embodiments, one or more agents of a plurality each independently comprise an antibody moiety that can interact hFcγRIIA on dendritic cells. In some embodiments, agents of a plurality can recruit immune cells. In some embodiments, one or more agents of a plurality each independently comprise an antibody moiety that can recruit an immune cell. In some embodiments, one or more agents of a plurality can recruit immune cells that inhibit, kill or remove a target (e.g., a small molecule, lipid, sugar, nucleic acid, microbe, bacteria, virus, foreign objects, diseased cells, etc.). In some embodiments, a target is a microbe. In some embodiments, a target is a virus. In some embodiments, a target is a SARS-CoV-2 virus. In some embodiments, agents of a plurality recruit immune cells. In some embodiments, agents of a plurality recruit NK cells. In some embodiments, agents of a plurality recruit macrophages. In some embodiments, agents of a plurality recruit dendritic cells.

In some embodiments, an agent induces, promotes, encourages, enhances, triggers, or generates ADCC and/or ADCP. In some embodiments, an agent induces, promotes, encourages, enhances, triggers, or generates ADCC and/or ADCP against a virus, e.g., a SARS-CoV-2 virus. In some embodiments, one or more agents of a plurality can induce, promote, encourage, enhance trigger or generate ADCC and/or ADCP. Those skilled in the art appreciate that technologies of the present disclosure may provide various types of immune activities and/or responses, including those involved in inhibition, killing and/or removal of viruses and cells infected thereby (in some cases, alternative to or in addition to ADCC and/or ADCP). In some embodiments, an agent induces, promotes, encourages, enhances, triggers, or generates long-term immunity (e.g., one or more vaccination effects). In some embodiments, an agent induces, promotes, encourages, enhances, triggers, or generates long-term immunity (e.g., one or more vaccination effects) against SARS-CoV-2. In some embodiments, technologies of the present disclosure provide long-term immunity. In some embodiments, a long-term immunity comprises memory T cells. In some embodiments, a long-term immunity comprises memory B cells. In some embodiments, a long-term immunity comprises memory T or B cells. In some embodiments, technologies of the present disclosure provide memory T and/or B cells against a target. In some embodiments, technologies of the present disclosure provide memory T and/or B cells against SARS-CoV-2. In some embodiments, one or more agents of a plurality can induce, promote, encourage, enhance, trigger or generate ADCC and/or ADCP, e.g., against SARS-CoV-2. In some embodiments, one or more agents of a plurality can induce, promote, encourage, enhance, trigger or generate long-term immunity, e.g., against SARS-CoV-2. In some embodiments, one or more agents of a plurality can provide memory T and/or B cells against SARS-CoV-2 when administered to a subject through one or more immunological processes.

In some embodiments, agents of a plurality comprise enriched levels of one or more types of antibody moieties. In some embodiments, one or more IgG isotypes are enriched in the composition. In some embodiments, IgG1 is enriched. In some embodiments, IgG2 is enriched. In some embodiments, IgG3 is enriched. In some embodiments, IgG4 is enriched. In some embodiments, two or three of IgG1, IgG2, IgG3, and IgG4 are enriched. In some embodiments, IgG1 and IgG2 are enriched. In some embodiments, enrichment is relative to a suitable reference. In some embodiments, a reference is serum of a subject (e.g., to whom an agent or composition is to be administered). In some embodiments, a reference is relevant levels in a population, e.g., a human population. In some embodiments, a reference is IVIG.

In some embodiments, antibody moieties in agents and/or compositions are or comprise structure features of recruited antibodies by antibody binding moieties. In some embodiments, antibody moieties in agents and/or compositions have properties and/or activities of recruited antibodies by antibody binding moieties.

In some embodiments, at least about 50%, 55%, 60%, 65%, 70%, 75%, 80%, 85%, 90%, 95%, 96%, 97%, 98%, or 99% of all agents, or substantially all agents, that comprise an antibody moiety and a target binding moiety in a composition are agents of a plurality.

In some embodiments, provided agents comprising antibody moieties can provide comparable or better safety profiles and/or therapeutic effects compared to serum derived antibodies obtained from subjects infected by SARS-CoV-2, e.g., those who have recovered or are recovering from COVID-19. In some embodiments, provided agents can be prepared from readily available antibodies, e.g., “off-the-shelf” IVIG and target binding moieties, and can be manufactured at much larger scale and/or much lower cost.

In some embodiments, the present disclosure provides an agent having the structure of

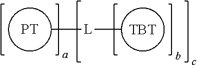

or a pharmaceutically acceptable salt thereof, wherein each PT is independently a partner moiety, and each other variable is independently as described herein (as appreciated by those skilled in the art, for each variable, an embodiment may be independently selected and then combined with embodiments independently selected for other variables). In some embodiments, each of a, b, and c is 1. In some embodiments, a partner moiety is or comprises a detection, diagnosis or therapeutic agent. In some embodiments, a partner moiety is a detectable moiety. In some embodiments, a detectable moiety is or comprises a fluorescent moiety. In some embodiments, a detectable moiety is or comprises a biotin moiety.

### 4. Uses, Formulation and Administration

### Pharmaceutically Acceptable Compositions

According to another embodiment, present disclosure provides a composition comprising an agent described herein or a pharmaceutically acceptable derivative thereof and a pharmaceutically acceptable carrier, adjuvant, or vehicle. In some embodiments, the present disclosure provides a pharmaceutical composition that comprises or delivers an agent, e.g., a MATE agent, an ARM agent, etc., of the present disclosure and a pharmaceutically acceptable carrier. In some embodiments, the present disclosure provides a pharmaceutical composition that comprises or delivers a therapeutically effective amount of an agent, e.g., a MATE agent, an ARM agent, etc., of the present disclosure and a pharmaceutically acceptable carrier. In some embodiments, an amount of an agent in a composition is such that it is effective to direct antibodies selectively to targets, e.g., diseased cells (e.g., SARS-CoV-2 infected cells), and/or induce antibody-directed activities, e.g., cell-mediated immunity such as cytotoxicity. In certain embodiments, an amount of an agent in a composition is such that is effective to direct antibodies selectively to cells expressing a SARS-CoV-2 spike protein or a fragment thereof, and induce antibody-directed activities, e.g., cell-mediated cytotoxicity, in a biological sample or in a subject (e.g., a SARS-CoV-2 infected patient). In certain embodiments, a composition is formulated for administration to a patient in need of such composition. In some embodiments, a composition is formulated for oral administration to a patient. In some embodiments, agents may be provided in various forms, e.g., various pharmaceutically acceptable salt forms. In some embodiments, a composition may comprise two or more forms of an agent.

In some embodiments, a pharmaceutically acceptable carrier, adjuvant, or vehicle is a non-toxic carrier, adjuvant, or vehicle that does not destroy the pharmacological activity of an agent with which it is formulated. Pharmaceutically acceptable carriers, adjuvants or vehicles that may be include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In some embodiments, a pharmaceutically acceptable derivative is a non-toxic salt, ester, salt of an ester or other derivative of an agent that, upon administration to a recipient, is capable of providing, either directly or indirectly, an agent or an active metabolite or residue thereof.

Compositions may be administered orally, parenterally, by inhalation spray, topically, rectally, nasally, buccally, vaginally or via an implanted reservoir. In some embodiments, parenteral administration includes subcutaneous, intravenous, intramuscular, intra-articular, intra-synovial, intrasternal, intrathecal, intrahepatic, intralesional and intracranial injection or infusion techniques. In some embodiments, compositions are administered orally, intraperitoneally or intravenously. Sterile injectable forms of compositions may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

In some embodiments, a bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

In some embodiments, pharmaceutically acceptable compositions may be administered in the form of suppositories for rectal administration. In some embodiments, these can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

In some embodiments, pharmaceutically acceptable compositions may be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, pharmaceutically acceptable compositions may be formulated in a suitable ointment containing the active component suspended or dissolved in one or more carriers. Carriers for topical administration of agents of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, pharmaceutically acceptable compositions are formulated for oral administration. Such formulations may be administered with or without food. In some embodiments, pharmaceutically acceptable compositions are administered without food. In other embodiments, pharmaceutically acceptable compositions are administered with food.

Amounts of agents that may be combined with the carrier materials to produce a composition in a single dosage form will vary depending upon the host treated, the particular mode of administration. In some embodiments, provided compositions are formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of an specific agent employed, the age, body weight, general health, sex, diet, time of administration, rate of excretion, drug combination, and the judgment of the treating physician and the severity of the particular disease being treated. The amount of an agent of the present disclosure in the composition will also depend upon a particular agent in the composition.

### Uses of Provided Agents and Compositions

In some embodiments, when contacted with its target, provided agents e.g., various MATE or ARM compounds, form complexes with antibodies and Fc receptors, e.g., those of various immune cells.

In some embodiments, the present disclosure provides a complex comprising:
an agent comprising:
   an antibody moiety,
   a target binding moiety, and
   optionally a linker moiety, and
an Fc receptor.
   In some embodiments, an antibody binding moiety is or comprises a Fc region. In some embodiments, an antibody moiety is or comprises IgG1. In some embodiments, an antibody moiety is or comprises IgG2. In some embodiments, an antibody moiety is or comprises IgG3. In some embodiments, an antibody moiety is or comprises IgG4.

In some embodiments, the present disclosure provides a complex comprising:
an agent comprising:
   an antibody binding moiety,
   a target binding moiety, and
   optionally a linker moiety,
an Fc region, and
an Fc receptor.
   In some embodiments, an antibody binding moiety is a universal antibody binding moiety.

In some embodiments, the present disclosure provides a plurality of complexes, each independently comprising:
an agent comprising:
   an antibody moiety,
   a target binding moiety, and
   optionally a linker moiety, and
an Fc receptor.

In some embodiments, the present disclosure provides a plurality of complexes, each independently comprising:
an agent comprising:
   an antibody moiety,
   a target binding moiety, and
   optionally a linker moiety, and
an Fc receptor,
   wherein Fc regions of the complexes are of antibodies and/or fragments thereof toward different antigens or proteins.

In some embodiments, the present disclosure provides a plurality of complexes, each independently comprising:
an agent comprising:
   an antibody binding moiety,
   a target binding moiety, and
   optionally a linker moiety,
an Fc region, and
an Fc receptor,
   wherein Fc regions of the complexes are of antibodies and/or fragments thereof toward different antigens or proteins.

In some embodiments, a plurality of complexes comprise a plurality of agents as described herein with a plurality of Fc receptors, and/or a plurality of Fc regions.

In some embodiments, a complex further comprises a target, e.g., a virus or a cell infected thereby. In some embodiments, a complex comprises a SARS-CoV-2 virus. In some embodiments, a complex comprise a cell infected by a SARS-CoV-2 virus. In some embodiments, a complex comprises a cell expressing a SARS-CoV-2 spike protein or a fragment thereof.

In some embodiments, Fc regions are of Fc regions of antibodies (e.g., antibodies recruited antibodies by agents comprising antibody binding moieties, antibody moieties in provided agents, etc.). In some embodiments, Fc regions of the complexes are of antibodies and/or fragments thereof toward different proteins. In some embodiments, one or more Fc regions are of endogenous antibodies and/or fragments thereof. In some embodiments, an Fc region is an Fc region of IgG1. In some embodiments, an Fc region is an Fc region of IgG2. In some embodiments, an Fc region is an Fc region of IgG3. In some embodiments, an Fc region is an Fc region of IgG4. In some embodiments, the present disclosure provides a plurality of complexes, wherein one or more complexes independently comprise an Fc region of IgG1. In some embodiments, the present disclosure provides a plurality of complexes, wherein one or more complexes independently comprise an Fc region of IgG2. In some embodiments, the present disclosure provides a plurality of complexes, wherein one or more complexes independently comprise an Fc region of IgG3. In some embodiments, the present disclosure provides a plurality of complexes, wherein one or more complexes independently comprise an Fc region of IgG4. In some embodiments, the present disclosure provides a plurality of complexes, wherein one or more complexes independently comprise an Fc region of IgG1, and one or more complexes independently comprise an Fc region of IgG2. In some embodiments, the present disclosure provides a plurality of complexes, wherein one or more complexes independently comprise an Fc region of IgG1, one or more complexes independently comprise an Fc region of IgG2, one or more complexes independently comprise an Fc region of IgG3, and one or more complexes independently comprise an Fc region of IgG4. In some embodiments, one or more complexes independently comprise a SARS-CoV-2 virus, and/or one or more complexes independently comprise a cell infected by SARS-CoV-2.

Without the intention to be bound by any theory, in some embodiments, provided technologies can deliver antibodies (e.g., through recruitment (e.g., antibody binding moieties) or by including antibody moieties) to an entity expressing a SARS-CoV-2 spike protein (unless otherwise indicated, including mutants thereof (e.g., those in viruses and/or infected cells)) or a fragment thereof (e.g., a SARS-CoV-2 virus, a cell infected by a SARS-CoV-2 virus, etc.). In some embodiments, antibodies reduce, inhibit or prevent interaction of SARS-CoV-2 viruses with other cells (e.g., mammalian cells that can be infected), in some embodiments, through disrupting, inhibiting or preventing interactions between SARS-CoV-2 spike proteins and cell proteins, e.g., receptors such as ACE2. In some embodiments, antibodies can induce, recruit, promote, encourage, or enhance one or more immune activities to inhibit, suppress, kill, or remove SARS-CoV-2 viruses and/or celled infected thereby. In some embodiments, antibodies can recruit dendritic cells. In some embodiments, a complex, e.g., a complex comprising a virus (e.g., a SARS-CoV-2 virus), an agent (e.g., an ARM agent comprising an antibody binding moiety, a target binding moiety and a linker as described herein) and an antibody moiety (either recruited antibody by an ARM agent or an antibody moiety in an agent; in some embodiment, such an antibody moiety is or comprises IgG2 which in some instances may have stronger binding to hFcyRIIA) binds hFcyRIIA on dendritic cells, and is internalized. Fragments of a virus, e.g., proteins and/or fragment thereof, are presented to immune cells (e.g., T cells) to provide long term immunity. In some embodiments, a complex comprising a virus-infected cell instead of a virus may similarly provide long term immunity. In some embodiments, provided technologies can provide long-term immunity (e.g., one or more vaccination effects). In some embodiments, provided technologies provide memory T and/or B cells against SARS-CoV-2.

In some embodiments, the present disclosure provides methods for inducing, promoting, encouraging, enhancing, triggering, or generating an immune response toward an infectious entity, e.g., a virus like SARS-CoV-2, comprising administering to a subject infected thereby an agent or a composition as described herein. In some embodiments, an immune response is or comprises ADCC. In some embodiments, an immune response is or comprises ADCP. In some embodiments, an immune response comprises ADCC and ADCP. In some embodiments, an immune response is or comprises long-term immunity. In some embodiments, an immune response is or comprises memory T and/or B cells. In some embodiments, a single dose is administered. In some embodiments, multiple doses are administered. In some embodiments, dosing intervals are about or not less than 1, 2 or 3 weeks, or about or not less than 1, 2, 3, 4, 5, 6, 7, 8, 9, 11 or 12 months, or about or not less than 1, 2, 3, 4, or 5 years. In some embodiments, at least one dosing interval is not less than 1, 2 or 3 weeks, or not less than 1, 2, 3, 4, 5, 6, 7, 8, 9, 11 or 12 months, or not less than 1, 2, 3, 4, or 5 years. In some embodiments, each dosing interval is independently not less than 1, 2 or 3 weeks, or not less than 1, 2, 3, 4, 5, 6, 7, 8, 9, 11 or 12 months, or not less than 1, 2, 3, 4, or 5 years. In some embodiments, a dosing interval is not less than 1 week. In some embodiments, a dosing interval is not less than 2 weeks. In some embodiments, a dosing interval is not less than 3 weeks. In some embodiments, a dosing interval is not less than 4 weeks. In some embodiments, a dosing interval is not less than 1 month. In some embodiments, a dosing interval is not less than 2 months. In some embodiments, a dosing interval is not less than 3 months. In some embodiments, a dosing interval is not less than 6 months. In some embodiments, a dosing interval is not less than or about 1 year. In some embodiments, a dosing interval is not less than or about 2 year. In some embodiments, a dosing interval is not less than or about 3 years. In some embodiments, a dosing interval is not less than or about 4 years. In some embodiments, a dosing interval is not less than or about 5 years.

In some embodiments, recruited antibodies or antibody moieties can induce, promote, encourage, enhance, trigger, or generate long-term immunity (e.g., after the initial ADCC and/or ADCP after an infection, or after 1, 2, 3, 4 or weeks, or after 1, 2, 3, 4, 5, 6, 7, 8, 9, 10, 11, 12 or more months after a last dose of an agent or a composition). In some embodiments, technologies of the present disclosure provide long-term immunity, e.g., toward SARS-CoV-2. In some embodiments, technologies of the present disclosure provide immunity in a period of time (e.g., 1, 2, 3, 4, 5, 6, 7, 8, 9, 10, 11, 12 or more months) after administration of an agent (in some embodiments, if multiple doses are administered as a regimen, after the first, the first several, or the last dose(s)) or a composition of the present disclosure. In some embodiments, a period of time is 6 months or more. In some embodiments, it is 7 months or more. In some embodiments, it is 8 months or more. In some embodiments, it is 9 months or more. In some embodiments, it is 10 months or more. In some embodiments, it is 11 months or more. In some embodiments, it is 1 year or more. In some embodiments, it is 2 years or more. In some embodiments, it is 3 years or more. In some embodiments, it is 4 years or more. In some embodiments, it is 5 years or more. In some embodiments, provided technologies can provide memory T or B cells against a target, e.g., SARS-CoV-2.

In some embodiments, the present disclosure provides a method for inhibiting, killing or removing a virus, e.g., SARS-CoV-2 virus, comprising administering to a subject infected thereby an effective amount of an agent or a composition. As appreciated by those skilled in the art, in some embodiments, an infected subject may not display a symptom (asymptomatic). In some embodiments, an agent or composition is administered before an infected subject displays a relevant symptom and/or when symptoms are considered mild (e.g., to prevent virus spreading, to prevent development of symptoms, and/or to prevent worsening of infection and/or overall condition of a subject). In some embodiments, a subject displays one or more symptoms considered medically “mild.” It is reported that common symptoms of SARS-CoV-2 infection/COVID-19 may be fever, tiredness, difficulty breathing, and/or dry cough. It is also reported that some subjects may have aches and pains, nasal congestion, runny nose, sore throat, loss of taste, loss of smell, and/or diarrhea. In some embodiments, symptoms are mild and begin gradually. In some embodiments, some subjects become infected but don't develop any symptoms and don't feel unwell. In some embodiments, a subject is seriously ill and develops difficulty breathing. In some embodiments, a subject is hospitalized. In some embodiments, provided agents and/or compositions are administered to subjects without symptoms, with mild symptoms, not hospitalized and/or hospitalized.

In some embodiments, the present disclosure provides a method for preventing and/or treating a condition, disorder or disease associated with an infection, e.g., a SARS-CoV-2 infection, comprising administering to a subject suffering therefrom a provided agent or composition. In some embodiments, the present disclosure provides a method for treating COVID-19, comprising administering to a subject suffering therefrom a provided agent or composition. In some embodiments, the present disclosure provides a method for inhibiting, killing or removing a virus, e.g., a SARS-CoV-2 virus, comprising contacting a virus, e.g., a SARS-CoV-2 virus, with a provided agent or composition. In some embodiments, the present disclosure provides a method for disrupting or reducing an interaction between a cell and a virus, e.g., a SARS-CoV-2 virus, comprising contacting a virus, e.g., a SARS-CoV-2 virus, with a provided agent or composition. In some embodiments, the present disclosure provides a method for disrupting or reducing an infection of a virus, e.g., a SARS-CoV-2 virus, of a cell, comprising contacting a virus, e.g., a SARS-CoV-2 virus, with a provided agent or composition. In some embodiments, the present disclosure provides a method for inhibiting, killing or removing a cell infected by a virus, e.g., a SARS-CoV-2 virus, comprising contacting the cell with a provided agent or composition. In some embodiments, provided agents or compositions are utilized in amounts effective to provide desired effects. As described herein, in some embodiments, immune cells, such as various NK cells, may be utilized together with provided agents and/or compositions, and may be administered prior to, concurrently with, or subsequently to provided agents and/or compositions. In some embodiments, a provided method is performed/started during an early phase of an infection or an associated condition, disorder or disease (e.g., COVID-19). In some embodiments, a provided method is performed/started before a subject generates strong immune activities. In some embodiments, a provided method is performed/started before a subject has acute respiratory distress syndrome (ARDS). In some embodiments, an agent is administered during an early phase of an infection or an associated condition, disorder or disease (e.g., COVID-19). In some embodiments, an agent is administered before a subject generates strong immune activities. In some embodiments, an agent is administered before a subject has ARDS.

In some embodiments, the present disclosure provides prophylactic methods for disrupting, reducing or preventing infection. In some embodiments, the present disclosure provides a method for disrupting, reducing or preventing a viral infection, e.g., an SARS-CoV-2 infection, comprising contacting a virus, e.g., a SARS-CoV-2 virus, with an effective amount of an agent or a composition of the present disclosure. In some embodiments, the present disclosure provides prophylactic methods for disrupting, reducing or preventing infection in advance of exposure. In some embodiments, the present disclosure provides a method for disrupting, reducing or preventing an SARS-CoV-2 infection, comprising contacting a SARS-CoV-2 virus with an agent or a composition as described herein. In some embodiments, the present disclosure provides a method for disrupting, reducing or preventing SARS-CoV-2 infection in a population, comprising administering to individual subjects in the population a therapeutically effective amount of an agent or a composition as described herein. In some embodiments, an agent or composition is administered to a subject before the subject is exposed to or contacts an infectious entity, e.g., before the subject is exposed to a virus like a SARS-CoV-2 virus. In some embodiments, an agent or composition is administered to a subject before the subject is infected. As appreciated by those skilled in the art, various technologies are available for assessing viral infection, e.g. SARS-CoV-2 infection, and/or conditions, disorders or diseases associated therewith (e.g., those based on nucleic acid and/or protein detection, imaging (e.g., X-ray, CT, etc.), those according to guidelines of various government and/or private organizations (e.g., US CDC, WHO, etc.), etc.). In some embodiments, the present disclosure provides a method for disrupting, reducing or preventing a viral infection, e.g., SARS-CoV-2 infection in a population, comprising administering to individual subjects in the population an effective amount of agent or a composition of the present disclosure. In some embodiments, the present disclosure provides a method for disrupting, reducing or preventing a viral infection, e.g., SARS-CoV-2 infection, comprising administering to a subject susceptible thereto an effective amount of an agent or a composition of the present disclosure. In some embodiments, the present disclosure provides a method for disrupting, reducing or preventing a viral infection, e.g., a SARS-CoV-2 infection, comprising administering to a subject susceptible thereto an effective amount of an agent or a composition of the present disclosure. In some embodiments, an infection is a re-infection. In some embodiments, a subject, e.g., a subject in a population, is more susceptible to infection, at higher risk of infection, or is more likely to develop serious illness when infected (e.g., senior people (e.g., with age of 60, 70, 80 or more), or those with underlying medical problems (e.g., high blood pressure, heart problems, diabetes, etc.)). In some embodiments, a subject is a healthcare provider. In some embodiments, a subject is a frontline healthcare worker. In some embodiments, a subject is in contact or is in close proximity to an infected subject. In some embodiments, a subject is a healthcare worker who treats an infected patient. In some embodiments, a subject is of age 50, 55, 60, 65, 70, 75, 80, 85, 90 or more. In some embodiments, a subject is a person who lives in a nursing home or long-term care facility. In some embodiments, a subject has one or more underlying medical conditions, e.g., asthma, diabetes, high blood pressure, heart disease, etc. In some embodiments, a condition is chronic lung disease. In some embodiments, a condition is moderate to severe asthma. In some embodiments, a condition is a heart condition. In some embodiments, a subject is immunocompromised (as appreciated by those skilled in the art, can be caused by many conditions/factors, e.g., a medical treatment (a cancer treatment), smoking, bone marrow or organ transplantation, immune deficiencies, HIV or AIDS (particularly if poorly controlled), prolonged use of certain medications (e.g., corticosteroids and other immune weakening medications), etc.). In some embodiments, a subject is a cancer patient (e.g., immunocompromised). In some embodiments, a condition is obesity. In some embodiments, a condition is severe obesity (BMI>=40). In some embodiments, a condition is renal failure. In some embodiments, a condition is a liver disease. In some embodiments, prophylactic uses may comprise one, two or more doses. In some embodiments, multiple doses are administered. In some embodiments, one or more dose intervals are not more than 1, 2, 3, 4, 5, 6, 7, 8, 9, or 10 times of the half-life of an administered agent (which, as appreciated by those skilled in the art, can be assessed using a number of technologies). In some embodiments, one or more dose intervals are about or not more than 1, 2, 3, 4, 5, 6, 7, 8, 9, 10, 11, 12, 13, 14, 15, 16, 17, 18, 19, 20, 21, 22, 23, or 24 hours, or about or no more than 1, 2, 3, 4, 5, 6, 7, 8, 9, or 10 days. In some embodiments, each dose interval is independently about or not more than 1, 2, 3, 4, 5, 6, 7, 8, 9, 10, 11, 12, 13, 14, 15, 16, 17, 18, 19, 20, 21, 22, 23, or 24 hours, or about or no more than 1, 2, 3, 4, 5, 6, 7, 8, 9, or 10 days. In some embodiments, an agent or composition is administered once, twice, or thrice per day, or once every 2, 3, 4, 5, 6, or 7 days.

In some embodiments, the present disclosure provide technologies are useful against various viruses, e.g., for providing immunity, inhibiting, killing or removing viruses and/or cells infected thereby, preventing and/or treating conditions, disorders or diseases associated with viral infections, disrupting, reducing or preventing infections, etc. as described herein. In some embodiments, provided technologies can target two or more viruses. In some embodiments, provided technologies can target two or more or all coronaviruses that infect humans as described herein, e.g., SARS-CoV, SARS-CoV-2 and/or MERS-CoV. In some embodiments, provided technologies are useful for against SARS-CoV. In some embodiments, provided technologies are useful for against SARS-CoV-2. In some embodiments, provided technologies are useful for against MERS-CoV. In some embodiments, provided technologies are useful for against SARS-CoV and SARS-CoV-2. In some embodiments, provided technologies are useful for against SARS-CoV, SARS-CoV-2 and MERS-CoV.

In some embodiments, cells are mammalian cells. In some embodiments, cells are human cells. In some embodiments, cells are of the respiratory system.

In some embodiments, the present disclosure provides pharmaceutical compositions comprising or delivering a provided agent or a pharmaceutically acceptable salt thereof and a pharmaceutically acceptable carrier. In some embodiments, provided technologies are administered to subjects in pharmaceutical compositions.

### Combination Therapies

In some embodiments, provided technologies are administered together with one or more additional therapeutic agents and/or technologies. In some embodiments, useful additional therapeutic agents and/or technologies for combination are those that have been utilized to treat a condition, disorder or disease associated with viral infection, particularly infection by SARS-CoV-2.

In some embodiments, an additional therapeutic agent is or comprises immune cells. In some embodiments, immune cells are or comprise macrophages. In some embodiments, immune cells are or comprise NK cells. In some embodiments, immune cells are engineered cells. In some embodiments, immune cells are prepared in vitro. For example, in some embodiments, NK cells are or comprise engineered cells. In some embodiments, NK cells are or comprise allogeneic NK cells. In some embodiments, NK cells are or comprises peripheral blood-derived NK cells. In some embodiments, NK cells are or comprises cord blood-derived NK cells. In some embodiments, immune cells are or comprise MG4101 cells. In some embodiments, immune cells are or comprises CB-NK cells.

In some embodiments, immune cells are administered concurrently with provided agents; in certain embodiments, in the same composition. In some embodiments, immune cells are administered prior to provided agents. In some embodiments, immune cells are administered subsequently to provided agents.

In some embodiments, the present disclosure provides a composition comprising an agent and a population of immune cells. In some embodiments, such a composition is a pharmaceutical composition. In some embodiments, a population of immune cells comprises NK cells. In some embodiments, a population of immune cells are a population of NK cells. In some embodiments, NK cells are allogeneic NK cells. In some embodiments, NK cells are or comprise peripheral blood-derived NK cells. In some embodiments, NK cells are or comprise MG4101 NK cells. In some embodiments, NK cells are or comprise CB-NK cells. In some embodiments, NK cells are or comprise cord blood-derived NK cells.

Various immune cells, particularly NK cells, may be utilized together with agents described herein to treat various conditions, disorders or diseases including COVID-19. Such cells may be administered prior to, concurrently with, and/or subsequent to agents described herein, e.g., MATEs, ARMs, etc. In some embodiments, such cells, e.g., NK cells, are administered concurrently with an agent, e.g., a MATE agent, an ARM agent, etc., in the same composition comprising both NK cells and an agent. In some embodiments, such cells, e.g., NK cells, are administered concurrently with an agent, e.g., a MATE agent, an ARM agent, etc., in separate compositions, e.g., one composition comprising NK cells but no agents, and one composition comprising an agent but no NK cells.

As appreciated by those skilled in the art, useful immune cells such as NK cells may be from various sources and/or be engineered in a number of ways. For example, in some embodiments, NK cells are derived from stem cells. In some embodiments, NK cells are derived from iPSC lines. In some embodiments, NK cells are derived from a clonal master iPSC line. In some embodiments, NK cells are engineered to express certain receptors, e.g., a high-affinity, optionally non-cleavable CD16 receptor. In some embodiments, NK cells are engineered to express chimeric antigen receptors (CARs). In some embodiments, NK cells are CAR-NK cells. In some embodiments, NK cells are engineered to express cytokine receptor. In some embodiments, NK cells comprise a IL-15 receptor fusion that enhances the persistence and expansion capabilities without requiring co-administration of cytokine support. In some embodiments, NK cells are engineered to prevent expression of certain cell proteins, e.g., certain cell surface proteins. In some embodiments, NK cells are or comprise memory-like NK cells. In some embodiments, NK cells are or comprise pre-activated, memory-like NK cells enriched for CD56 and depleted from CD3 expressing cells. In some embodiments, NK cells are derived from placenta. In some embodiments, NK cells are donor NK cells. In some embodiments, NK cells are haploidentical donor NK cells. In some embodiments, NK cells are mismatched donor NK cells. In some embodiments, NK cells are related donor NK cells, e.g., mismatched related donor NK cells. In some embodiments, NK cells are unrelated donor NK cells. In some embodiments, NK cells are derived from a subject, e.g., a patient. In some embodiments, provided technologies comprise an innate cell engager, e.g., an innate cell engager binding to innate cells (e.g., NK cells and macrophages) while binding simultaneously to specific virally infected cells. In some embodiments, NK cells are derived from cord blood stem and progenitor cells. In some embodiments, NK cells are derived with modulation of a signaling pathway, e.g., the Notch signaling pathway. In some embodiments, nanoparticles are utilized to improve and/or sustain growth of NK cells. In some embodiments, as described herein, NK cells are generated ex vivo. In some embodiments, NK cells may be cryopreserved and stored in multiple doses as off-the-shelf cell therapy. Examples of certain immune cell technologies (e.g., NK cell technologies) include those utilized by Fate Therapeutics, NantKwest Inc., Celularity, Inc., GC Pharma, Sorrento Therapeutics, Inc., Affimed GmbH/MD Anderson Cancer Center, Gamida Cell Ltd., Nohla Therapeutics, Kiadis Pharma N.V., NKMax, Glycostem Therapeutics BV, GC LabCell, etc. Those skilled in the art will appreciate that, which they can be optionally utilized, antibodies and/or CARs toward specific antigens utilized in certain such technologies may not be required in provided technologies comprising agents as described herein.

### EXEMPLIFICATION

Various agents are prepared utilizing chemical and/or biological technologies in accordance with the present disclosure. Agents are assessed using available assays, e.g., those described in Zhang et al., https://doi.org/10.1101/2020.03.19.999318; Xia, S., Zhu, Y., Liu, M. et al. Fusion mechanism of 2019-nCoV and fusion inhibitors targeting HR1 domain in spike protein. Cell Mol Immunol (2020). https://doi.org/10.1038/s41423-020-0374-2; Cite as: L. Cao et al., De novo design of picomolar SARS-CoV-2 mini-protein inhibitors. Science 370, 426-431 (2020); etc. for their properties and activities, including binding to SARS-CoV-2 spike proteins, inhibition, reduction and prevention of binding and/or infection of cells, inhibition, killing, and removal of SARS-CoV-2 viruses and/or cells infected thereby, etc. As demonstrated herein, provided agents can provide various useful properties and/or activities. Certain technologies for assessing/characterizing agents, compounds, compositions, methods, etc. are described below as examples.

### Example 1. Provided Agents can Bind to SARS-CoV-2 Spike Protein

In some embodiments, interactions were assessed utilizing ELISA technologies. In some embodiments, binding affinity of various agents to COVID-19 Spike RBD and Trimer protein was determined by ELISA. A binding assay was performed comprising the following steps (All wash steps are 4×1 minute with 200 uL PBST; appreciated by those skilled in the art, one or more steps and/or parameters and/or conditions may be adjusted): (1) Coat assay plates with 100 uL Anti-His (for 1-2) at 5 ug/mL in PBS, incubate 1 hour at 37° C. without shaking. Wash. (2) Block plates with 200 uL 5% BSA in PBSTB, 1 hour (RT), shake at 400 rpm on plate shaker. Wash. (3) His-tagged (for 1-2) protein is added, 100 uL at 5 nM in PBSTB, 1 hour (RT), shake at 200 rpm. Wash. (4) an agent was added, 100 uL at a concentration range of 1-1,000 nM (10-10,000 nM for I-2) in PBSTB (I-26 or I-28 in its original solution as prepared), 1 hour (RT). Shake at 200 rpm. Wash. (5) Binding was detected with the addition of Anti-Myc antibody. Wash. Addition of Anti-mouse IgG2a-HRP (for I-2), Neutravidin-HRP; for I-26 or I-28, Anti-F(ab) HRP) (1:20,000), 100 uL in PBSTB, 1 hour (RT). Shake at 200 rpm. Wash. (5) Chemiluminescent substrate was added for 5 minutes. Shake at 200 rpm. The plate was then read on the BioTech plate reader.

In some embodiments, interactions are assessed by competition assays. In some embodiments, competition of ACE2 binding to Spike RBD by various agents was examined by ELISA. In some embodiments, a binding assay was performed using the following steps (All wash steps are 4×1 minute with 200 ul PBST; appreciated by those skilled in the art, one or more steps and/or parameters and/or conditions may be adjusted): (1) Assay plates were coated with 100 ul Neutravidin (1-24) or Anti-His at 5 ug/mL in PBS and incubated 1 hour at 37° C. without shaking. Wash. (2) Block plates with 200 ul 5% BSA in PBSTB, 1 hour (RT), shake at 400 rpm on plate shaker. Wash. (3) Biotin-(I-24) or His-tagged RBD protein is added, 100 uL at 5 nM in PBSTB, 1 hour (RT), shake at 200 rpm. Wash. (4) Fc-tagged ACE2 was added, 50 uL at a 2× concentration of 2 nM (1 nM final) in PBSTB along with 1-24, 50 uL of 2× at a concentration range of 2-2,000 nM (1-1,000 nM final) in PBSTB, 1 hour (RT), shake at 200 rpm. Wash. (5) Binding of Fc-tagged ACE2 was detected with the addition of Anti-Fc-HRP (1:15,000), 100 uL in PBSTB, 1 hour (RT), shake at 200 rpm. Wash. (5) Chemiluminescent substrate is added for 5 minutes, shake at 200 rpm. The plate was then read on the BioTech plate reader.

Among other things, the present disclosure demonstrates that certain agents of the disclosure can bind to COVID-19 spike proteins. In some embodiments, it was observed that MATE agents can bind to spike proteins and/or RBD domains thereof with much higher affinities than the corresponding target binding moiety polypeptides (either synthetic or recombinant) alone. Certain data were presented below:

| Agent | Description | Results (nM) |
|---|---|---|
| I-26 | Binding to Anchored Spike RBD | EC50 = 15.8 |
| I-26 | Binding to Anchored Spike Trimer | EC50 = 32.3 |
| I-27 | Binding to Anchored Spike RBD | EC50 = 4.0 |
| I-27 | Binding to Anchored Spike Trimer | EC50 = 15.9 |
| I-1 | Inhibition of ACE2 Binding to | IC50 >1000 nM, |
| | Spike RBD | 42% Inhibition @ |
| | | 1,000 nM |
| I-2 | Binding to Anchored Spike RBD | EC50 >10,000; |
| | | >10,000 |
| I-2 | Binding to Anchored Spike Trimer | EC50 = 1379, 592 |
| I-2 | Inhibition of ACE2 Binding to | IC50 >1000 nM, |
| | Spike RBD | 30% Inhibition @ |
| | | 1,000 nM |
| I-28-1 | Binding to Anchored Spike RBD | EC50 = 2.9 |
| I-28-1 | Binding to Anchored Spike Trimer | EC50 = 4.5 |
| I-28-2 | Binding to Anchored Spike RBD | EC50 = 3.4 |
| I-28-2 | Binding to Anchored Spike Trimer | EC50 = 4.6 |

In some embodiments, one or more of the following reagents and equipment were utilized. Those skilled in the art will appreciate that other suitable reagents and equipment may also be utilized in addition to or as an alternative to those listed below in accordance with the present disclosure.

### Materials

Bovine Serum Albumin (BSA) Heat Shock, Fraction V—American Bio AB01088-00100
Phosphate Buffered Saline (PBS)—Gibco 10010, (−)Ca (−)Mg, pH 7.4
Tween 20—Fisher Bioreagents BP337-500
PBST—PBS+0.05% (w/v) Tween 20, pH 7.4
PBSTB—PBST+0.1% (w/v) BSA, pH 7.4
Assay Plate, PE B&W Isoplate 96-well Flat Bottom, High Binding—Perkin Elmer 6005580
Neutravidin—Thermo 31000, 1 mg/ml
Anti-6× His tag Antibody—Abcam ab15149, Mouse Monoclonal, 1 mg/mL
Anti-Myc Antibody [9E11]—Abcam ab56, Mouse mAb 1 mg/mL
Biotinylated SARS-CoV-2 (COVID-19) S Protein RBD, His, Avitag—Acro Biosystems SPD-C82E9
SARS-CoV-2 (COVID-19) S Protein RBD, His Tag—Acro Biosystems SPD-C52H3
Human ACE2/ACEH Protein, Fc Tag—Acro Biosystems AC2-H5257
Biotinylated SARS-CoV-2 (COVID-19) S Protein, His, Avitag Tag, Super Stable Active Trimer—Acro
Biosystems SPN-C82E9
Biotinylated Human CD38 Protein, Avi Tag—Acro Biosystems CD8-H82E7
Goat Anti-Mouse IgG2a-HRP—SouthemBiotech 1080-05
SARS-CoV-2 (COVID-19) S Protein (R683A, R685A), His Tag, Super Stable Active Trimer—Acro Biosystems SPN-C52H9
SARS-CoV-2 (COVID-19) S Protein RBD, His Tag—Acro Biosystems SPD-C52H3
His-tagged Human CD38 Protein—Acro Biosystems CD8-H5224
Neutravidin-HRP—Invitrogen 31030
IVIG Human Immune Globulin Intravenous, Flebogamma—Grifols NDC 61935-0005-5, 100 mg/ml—Dilute to 1 mg/ml in PBS
F(ab)2 Fragment Goat Anti-Human IgG1 F(ab)2 HRP (Jackson Immuno Research, 109-036-097
F(ab)2 Fragment Goat Anti-Human IgG1 Fc gamma HRP—Jackson Immuno Research, 109-036-170
SuperSignal ELISA Pico Chemiluminescent Substrate—Thermo 37069

### Equipment

Benchmark OrbiShaker MP, Model BT1502
BioTek Synergy H1 Microplate Reader, Model H1MFG

Binding of agents were also demonstrated using Octet technologies. In some embodiments, provided technologies (e.g., agents, compositions, methods, etc.) are assessed using Bio-Layer Interferometry (BLI) technologies in accordance with the present disclosure. In some embodiments, provided MATE agents provided higher affinity compared to corresponding target binding moiety agents.

In some embodiments, binding of provided agents, e.g., 1-2, 1-28, etc., to COVID-19 Spike Trimer/RBD was measured by Bio-Layer Interferometry (BLI) using the Fortebio Octet 96e platform. In some embodiments, a binding assay was performed using the Anti-Penta His (HIS1K) biosensors with the following steps (appreciated by those skilled in the art, one or more steps and/or parameters and/or conditions may be adjusted): (1) 60 seconds of baseline; (2) 600 seconds of loading with 25 nM Trimer/RBD; (3) 60 seconds of baseline; (4) 300 seconds of quenching with 50 ug/mL of Biocytin (for I-2); (5) 60 seconds of baseline (for I-2); (6) 300 seconds of association with a dilution series of I-2 or I-28; (8) 300 seconds of dissociation in assay buffer. In some embodiments, the dilution series included seven concentrations of I-2: 10, 30, 100, 300, 1000, 3000, 10,000, or I-28: 3.9, 7.8, 15.625, 31.25, 62.5, 125 and 250 nM. The eighth tip in the column was run in assay buffer to serve as a reference sensor, the signal of which was subtracted from the sample sensors. Binding of I-2 or I-28 in the absence of Trimer/RBD protein was also run and subtracted as non-specific binding of I-2 or I-28 to the Anti-Penta-His tips. Data analysis was done using the Octet Data Analysis HT software. Processed data was globally fit to a 1:1 binding model for determination of the equilibrium dissociation constant (K_{D}).

In some embodiments, one or more of the following reagents and equipment were utilized. Those skilled in the art will appreciate that other suitable reagents and equipment may also be utilized in addition to or as an alternative to those listed below in accordance with the present disclosure.

### Materials

Bovine Serum Albumin (BSA) Heat Shock, Fraction V—American Bio AB01088-00100
Phosphate Buffered Saline (PBS)—Gibco 10010, (−) Ca (−) Mg, pH 7.4
Tween 20—Fisher Bioreagents BP337-500
Assay buffer—PBS, 0.1% (w/v) BSA, 0.05% (v/v) Tween 20, pH 7.4
SARS-CoV-2 (COVID-19) S Protein (R683A, R685A), His Tag, Super Stable Active Trimer—Acro Biosystems SPN-C52H9
SARS-CoV-2 (COVID-19) S Protein RBD, His Tag—Acro Biosystems SPD-C52H3
Anti-Penta-His (HIS1K) Dip and Read Biosensors—Forte'Bio 18-5120
Biocytin—Thermo B1592
Assay Plate—96-well black flat bottom, Greiner bio-one 655209

### Equipment

Fortebio Octet 96e

In some embodiments, binding of COVID-19 Spike Trimer/RBD to provided agents, e.g., I-24, I-2, etc., was measured by Bio-Layer Interferometry (BLI) using the Fortebio Octet 96e platform. In some embodiments, a binding assay was performed using the Protein A (ProA) (I-24) or Streptavidin (SA) (1-2) biosensors with the following steps: (1) 60 seconds of baseline; (2) 600 seconds of loading with 10 ug/ml Anti-Myc (1-24) or 300 seconds of loading with 100 nM 1-2; (3) 60 seconds of baseline; (4) 600 seconds of loading of 5 uM I-24 or 300 seconds of quenching with 50 ug/mL of Biocytin; (5) 60 seconds of baseline; (6) 180 seconds of association with a dilution series of Spike Trimer (1-24) or 300 seconds of association with a dilution series of Spike Trimer/RBD (1-2); (7) 180 seconds of dissociation in assay buffer. The dilution series consisted of seven concentrations of Trimer: 3.9, 7.8, 15.625, 31.25, 62.5, 125, and 250 nM; or seven concentrations of RBD: 7.8, 15.625, 31.25, 62.5, 125, 250 and 500 nM. The eighth tip in the column was run in assay buffer to serve as a reference sensor, the signal of which was subtracted from the sample sensors. Binding of Trimer/RBD in the absence of I-24 or I-2 was also run and subtracted as non-specific binding of Trimer/RBD to the Anti-Myc antibody or Streptavidin tips. Data analysis was done using the Octet Data Analysis HT software. Processed data was globally fit to a 1:1 binding model for determination of the equilibrium dissociation constant (K_{D}).

In some embodiments, one or more of the following reagents and equipment were utilized. Those skilled in the art will appreciate that other suitable reagents and equipment may also be utilized in addition to or as an alternative to those listed below in accordance with the present disclosure.

### Materials

Bovine Serum Albumin (BSA) Heat Shock, Fraction V—American Bio AB01088-00100
Phosphate Buffered Saline (PBS)—Gibco 10010, (−) Ca (−) Mg, pH 7.4
Tween 20—Fisher Bioreagents BP337-500
Assay buffer—PBS, 0.1% (w/v) BSA, 0.05% (v/v) Tween 20, pH 7.4
SARS-CoV-2 (COVID-19) S Protein (R683A, R685A), His Tag, Super Stable Active Trimer—Acro Biosystems SPN-C52H9
Protein A (ProA) Dip and Read Biosensors—Forte'Bio 18-5010
Anti-Myc Antibody [9E11]—Abcam ab56, Mouse mAb 1 mg/ml
SARS-CoV-2 (COVID-19) S Protein RBD, His Tag—Acro Biosystems SPD-C52H3
Streptavidin (SA) Dip and Read Biosensors—Forte'Bio 18-5019
Biocytin—Thermo B1592
Assay Plate—96-well black flat bottom, Greiner bio-one 655209

### Equipment

Fortebio Octet 96e

Certain results were presented below. As demonstrated herein, among other things provided MATE agents can provide surprisingly high-affinity binding to spike proteins compared to target (spike protein) binding peptide moiety which also demonstrated binding in some cases.

| | | K_{D} | Max Signal |
|---|---|---|---|
| Agent | Description | (nM) | (nm) |
| I-24 | Spike Trimer Binding to Anchored P227 | 2.26 | 1.15 |
| I-24 | LCB1 Binding to Anchored Spike RBD | No Binding | |
| I-25 | Spike Trimer Binding to Anchored P228 | 1.58 | 1.3 |
| I-25 | LCB3 Binding to Anchored Spike RBD | No Binding | |
| I-1 | Binding to Anchored Spike RBD | No Binding | |
| I-1 | Binding to Anchored Spike Trimer | No Binding | |
| I-2 | Binding to Anchored Spike RBD | 13.2, 5.1, 5.2 | 0.12, 0.09, 0.10 |
| I-2 | Binding to Anchored Spike Trimer | Weak Binding × 2 | |
| I-2 | Spike RBD Binding to Anchored KP2209 | 73.0 | 1.7 |
| I-2 | Spike Trimer Binding to Anchored KP2209 | 23.0 | 3.0 |
| I-28-1 | Binding to Anchored Spike RBD | 4.26 | 2.6 |
| (Batch No. 1) | | | |
| I-28-1 | Binding to Anchored Spike Trimer | <1.0 pM | 0.16 |
| I-28-2 | Binding to Anchored Spike RBD | 4.08 | 2.6 |
| (Batch No. 2) | | | |
| I-28-2 | Binding to Anchored Spike Trimer | <1.0 pM | 0.15 |

Additional data confirming binding to spike protein or fragment thereof are presented below:

| Agent | A | B | C |
|---|---|---|---|
| I-4 | 3.47 nM | | |
| I-5 | 1.35 nM | | |
| I-6 | | <1.0 pM | 0.38 nM |
| I-9 | 3.26 nM | | |
| I-10 | 4.27 nM | | |
| I-11 | | 0.34 nM | 0.79 nM |
| I-12-1 | 2.48 nM | | |
| (Batch 1) | | | |
| I-12-2 | 1.98 nM | | |
| (Batch 2) | | | |
| I-13 | 2.29 nM | | |
| I-14 | | <1.0 pM | 0.52 nM |
| I-16 | 3.05 nM | | |
| I-17 | 3.36 nM | | |
| I-18 | | <1.0 pM | 0.67 nM |

A: Octet, binding to immobilized Spike RBD, K_{D}; non-biotinylated ligand
B: Octet, binding to immobilized Spike RBD, K_{D}; biotinylated ligand
C: Elisa, binding to anchored Spike RBD, EC₅₀; biotinylated ligand

| | Elisa, binding to anchored |
|---|---|
| Agent | Spike trimer, EC₅₀ |
| I-29 | 0.50 nM |
| I-30 | 0.41 nM |
| I-31 | 0.53 nM |
| I-33 | 0.36 nM |
| I-34 | 0.46 nM |
| I35 | 0.54 nM |
| I-36 | 0.49 nM |
| I-32 | 0.87 nM |
| I-28-3 (Batch 3) | 3.8 nM |

### Example 2. Provided Technologies can Bind to Entities Comprising Spike Proteins

Among other things, provided technologies can bind to entities expressing spike proteins. For example, in some embodiments, provided agents recognize and bind to human cells expressing spike proteins. Certain useful procedures for assessing binding to spike protein-expressing cells by various agents are described below as an example. Those skilled in the art appreciate that various other suitable conditions, parameters, reagents, instruments, etc. may be utilized in accordance with the present disclosure.

Biotinylated compounds. For biotinylated agents 293 T cells stably expressing SARS-CoV2 Spike-GFP protein were incubated with a concentration range of compounds or biotinylated ACE2 as a control for 60 min at 37° C. Cells were washed twice RPMI 5% low IgG serum. Cells were then incubated with 10 ug/mL of Streptavidin APC and incubated for 30 min at 4° C. Cells were subsequently washed two times in RPMI 5% low IgG serum, resuspended in PBS 1% BSA, and acquired on Attune NxT flow cytometer. In some embodiments, mean fluorescence intensity of APC as well as % APC positive in GFP positive gate are reported as readouts in this assay.

MATE agents. 293T cells stably expressing SARS Cov2 Spike GFP protein on their surface were incubated with a concentration range of MATES for 60 min at 37° C. in RPMI 5% low IgG serum. Cells were washed twice using the same medium, and incubated with 1.6 ug/mL of polyclonal anti-human IVIG-Dylight 650 as a secondary reagent to detect antibody binding for 30 min at 4° C. Cells were washed twice in RPMI 5% low IgG serum, resuspended in PBS 1% BSA and acquired on Attune NxT flow cytometer. In some embodiments, mean fluorescence intensity of APC as well as % APC positive in GFP positive gate are reported as readouts in this assay.

Certain data were presented below, demonstrating that provided agents can bind to entities comprising spike proteins:

| Agent | I-29 | I-30 | I-31 | I-32 | I-33 | I-34 | I-35 |
|---|---|---|---|---|---|---|---|
| IC50% gated | 2.1 | 0.64 | 0.58 | 2.2 | 1.4 | 1.1 | 3.3 |
| [nM] | | | | | | | |
| IC50 Median | 6.7 | 3.7 | 4.7 | 9.5 | 6.3 | 4 | 6.7 |
| [nM] | | | | | | | |

| Agent | I-36 | I-28-1 | I-28-2 | I-28-3 | I-26 | I-27 | |
|---|---|---|---|---|---|---|---|
| IC50% gated | 2.9 | n/a | n/a | 0.75 | 1.3 | 0.28 | |
| [nM] | | | | | | | |
| IC50 Median | 4.9 | 0.22 | 0.77 | 1.6 | 1.6 | 0.13 | 0.55 |
| [nM] | | | | | | | |
| | | | | | | | 5.6 |

*n/a = not measured

### Example 3. Provided Technologies can Provide Immune Activities

In some embodiments, agents are assessed and confirmed their immune activities. For example, in some embodiments, agents are assessed in ADCP assays. In some embodiments, certain agents were assessed using the following protocol as examples; those skilled in the art appreciate that one or more steps and/or parameters and/or conditions may be adjusted. As demonstrated herein, provided technologies can recruit various immune activities including macrophages.

MATE agents were diluted in PBS (VWR Cat. #20012043) to 10× the starting concentration used in the assay into a 96 well polypropylene plate (Corning 3357). Agents were diluted in DMSO (MP 191418) to 1000× the starting concentration used in the assay into a 96 well polypropylene plate (Corning 3357). They were then serially diluted in ½ log increments to generate 8-12 concentrations in PBS (assay dependent). Biotinylated Human ACE2/ACEH Protein, His, Avitag™ (Acro AC2-H82E6) was diluted to 10× the starting concentration in PBS and serially diluted in ½ log increments to generate 8-12 concentrations in PBS.

SARS-CoV-2 spike protein RBD beads (Acro MBS-K002-2 mg) were counted and diluted to 30,000/10 uL in DMEM (Thermo Fisher Scientific 11995040) with 10% FBS and Penicillin Streptomycin (media). RBD beads were added to each well of a 96 well polypropylene plate (Corning 3357) 10 uL/well. Diluted test agents (MATE agents, compounds or Ace-2) were added to the beads, 10 uL/well, in duplicates. This mixture was incubated 15 min at 37° C.

Raw 264.7 macrophages (InvivoGen raw-sp) were grown in media. Cells were harvested by manual scraping (VWR 10062-908), counted and diluted to 635000/mL. To fluorescently detect binding, mouse anti-human IgG1 Hinge antibody (Southern Biotech 9052-30) was added to the macrophages 1.25 ug/mL. To fluorescently detect ACE-2 biotin, mouse anti-biotin (Jackson 200-542-211)) was added to the macrophages 1.25 ug/ml.

Cells with the appropriated detection antibody. Anti-Hinge was used for MATE agents; anti-biotin for biotinylated ACE2 and other compounds were added to complexed RBD beads & test agents, 80 uL/well. The wells were mixed by manually pipetting up and down. The plates were covered and incubated at 37° C. in an incubator for 2h.

At the end of the incubation period, assay was run on an Attune (Thermo Fisher) flow cytometer set to acquire 50 uL. PMT settings SSC 160, FSC 350; BL1 400. Events were captured using three gated regions: Free beads, Macrophages, and SSC shifted Macrophages. Data were graphed using Graphpad Prism and curves were fit using log(inhibitor) vs. response—Variable slope (four parameters).

Certain results from various runs were presented below (10 runs, EC50 (nM) and % free beads for each). As demonstrated herein, provided technologies can provide immune activities including ADCP activities, e.g., through recruiting macrophages to targets comprising spike proteins.

| | EC50 | % free | EC50 | % free | EC50 | % free | EC50 | % free | EC50 | % free |
|---|---|---|---|---|---|---|---|---|---|---|
| Agent | (nM) | beads | (nM) | beads | (nM) | beads | (nM) | beads | (nM) | beads |
| I-26 | 89 | 10 | 23 | 20 | | | | | | |
| I-27 | 4.5 | 5 | 15 | 25 | | | | | | |
| I-2 | 3.8 | 5 | 8.2 | 5 | | | | | | |
| I-28-3 | | | 10 | 20 | 56 | 0 | 0.12 | 5 | 1.5 | 20 |
| Ace2 | 4.8 | 5 | nd | | | | | | | |
| I-2 | | | 1.2 | 8.1 | 2.8 | 10.0 | | | | |
| I-28-3 | 2.4 | 10 | | | | | | | 0.4 | 0 |
| ACE2 | | | 0.64 | 8.2 | 0.5 | 14.4 | 3.1 | 16.3 | | |
| I-6 | | | 0.2 | 7.7 | 0.16 | 17.8 | | | | |
| I-11 | | | 1.2 | 7.4 | 0.43 | 19.1 | | | | |
| I-14 | | | 0.59 | 7.0 | 0.63 | 10.9 | | | | |
| I-18 | | | 1.4 | 7.3 | 19 | 15.2 | | | | |
| I-29 | | | | | | | 0.74 | 98 | 0.82 | 3.6 |
| I-30 | | | | | | | 0.71 | 6.2 | 1.1 | 1.5 |
| I-31 | | | | | | | 0.79 | 5.7 | 1.1 | 1.7 |
| I-32 | | | | | | | 1.2 | 5.9 | 1.2 | 3.1 |
| I-33 | | | | | | | 0.9 | 7.7 | 1 | 3.4 |
| I-34 | | | | | | | 0.84 | 7.8 | 1.1 | 2.4 |
| I-35 | | | | | | | 1.1 | 7.8 | 1.3 | 1.5 |
| I-36 | | | | | | | 1.1 | 8.5 | 0.8 | 2.6 |

### Example 4. Provided Technologies can Neutralize SARS-Cov-2 Virus

In some embodiments, provided technologies can neutralize viruses expressing spike proteins. In some embodiments, provided technologies neutralize SARS-Cov-2 virus. In some embodiments, provided technologies reduce, inhibit or prevent infection of entities, e.g., cells, by SARS-Cov-2 virus. Various technologies are available for assessing provided technologies for neutralizing viruses. A procedure is described below as an example. Those skilled in the art appreciate that one or more steps and/or parameters and/or conditions may be adjusted in accordance with the present disclosure.

For agents in DMSO, a 1000× stock of test compound is used to prepare working concentrations. Working concentrations are first diluted in DMSO using 2-fold dilutions, then diluted 1:100 in cell culture media. A 2× dilution is then made in cell culture media for addition of virus.

Control and agents in aqueous solutions are serially diluted 10-fold and ACE2 control agent diluted 3-fold in cell culture media to a 2× working concentration for addition of virus. Agents are typically prepared immediately prior to the start of experiments. Dilutions are not tested for homogeneity or concentration. Dose-dependent anti-viral effect are studied. Cells are infected with USA-WA1/2020 (SARS-CoV-2) virus at a MOI of 0.001 TCID₅₀/cell.

African green monkey kidney Vero E6 cells are cultured in 96 well plates prior to the day of the assay. Vero E6 cells are at greater than 90% confluency at the start of the study. Each of the agent concentrations are evaluated in triplicate. Agent concentrations to be tested are prepared and then incubated with an equal volume of virus for 60-90 minutes. Virus:test agent mixture is then transferred to Vero E6 cells and incubated for 60-90 minutes. Following the incubation, virus:test agent is removed, cells are washed and wells overlaid with 0.2 mL DMEM2 (DMEM with 2% FBS with compounds) and incubated in a humidified chamber at 37° C.±2° C. in 5±2% CO₂. At 48±6 hours post inoculation, cells are fixed and evaluated for the presence of virus.

Vero E6 cells are typically maintained in Dulbecco's Minimum Essential Medium with 10% fetal calf serum. All growth media contains heat-inactivated fetal calf serum and antibiotics. 2019 Novel Coronavirus, Isolate USA-WA1/2020 (SARS-CoV-2) are typically stored at approximately ≤−65° C. prior to use.

In some embodiments, agents are tested against wild type USA-WA1/2020 (SARS-CoV-2) in triplicate. Test and control agents are serially diluted then incubated with a standardized virus concentration of 0.001 TCID₅₀/cell at 37° C.±2° C. in 5.0%+1-1% CO₂ for 60-90 min. 100 μL of virus:test agent mixture is then transferred into respective wells of a 96-well plate which contains a monolayer of Vero E6 cells and incubated in a humidified chamber at 37° C.±2° C. in 5±2% CO2. for 60-90 min. Following the incubation, virus:test article is removed, cells are washed and wells overlaid with 0.2 mL DMEM2 (DMEM with 2% FBS with agents) and incubated in a humidified chamber at 37° C.±2° C. in 5±1% CO₂. After 24 or 48±6 hours, cells are fixed with paraformaldehyde and stained by anti-SARS-2 nucleoprotein monoclonal antibody (Sino Biological) followed by peroxidase-conjugated goat anti-mouse IgG (SeraCare). Wells are developed using TMB Substrate Solution and the reaction stopped by acidification. The ELISA plate is read at 450 nm on a spectrophotometer by ELISA plate reader. For each well, inhibition of virus is calculated as the percentage of reduction of the absorbance value in respect of the virus control by the following formula: percent inhibition=100−[(A450 of antibody dilution−A450 of cell control)/(A450 of virus control−A450 of cell control)]×100. Neutralizing titers/concentrations are defined as the reciprocal dilution that caused 50% reduction of the absorbance value of the virus control (50% A450 reduction).

In some embodiments, the following agents are utilized/assessed.

### Aqueous Solutions

| | | Amount of | Starting |
|---|---|---|---|
| | | aqueous solution | concentration |
| Agents | FW/MW | (volume, mg/mL) | on test plate |
| I-28-3 | 160,000 | 150.0 uL of 1.77 mg/mL | 100 μg/mL |
| I | 150,000 | 100.0 uL of 2 mg/mL | 100 μg/mL |
| 3 | 150,000 | 100.0 uL of 2 mg/mL | 100 μg/mL |
| 4 | 150,000 | 100.0 uL of 2 mg/mL | 100 μg/mL |
| 5 | 150,000 | 100.0 uL of 2 mg/mL | 100 μg/mL |
| 6 | 180,000 | 250.0 uL of 0.9 mg/mL | 100 μg/mL |
| ACE2 | 80,000 | Control 0.34 mg/mL 30 uL | 30 μg/mL |
| I-28-3 | 160,000 | 150.0 uL of 1.77 mg/mL | 100 μg/mL |
| I-26 | 160,000 | 33 ul 2.9 mg/ml = 95.7 ug | 50 μg/mL |
| I-27 | 160,000 | 35 ul 2.9 mg/ml = 101 ug | 50 μg/mL |
| I-28-1 | 150,000 | 5 ul of 0.99 mg/ml = 5 ug | 3.0 μg/mL |
| I-28-2 | 150,000 | 70 ul of 1.36 mg/ml = 95.2 ug | 50 μg/mL |

*Run twice on 2 separate plates in some cases.

### Supplied as Powder to be Dissolved in DMSO

| | Formula | | Volume (μl) of | Starting |
|---|---|---|---|---|
| | Weight (with | Amount | DMSO to add | concentration on |
| Agent | salts) | (mg) | for [10 mM] | test plate |
| I-1 | 6822 | 2.45 | 36 | 50 μM |
| I-4 | 6925 | 2.4 | 31.2 | 50 μM |
| I-5 | 6967 | 2.1 | 26.0 | 50 μM |
| I-6 | 7398 | 2.8 | 34.3 | 50 μM |
| I-9 | 7851 | 2.6 | 29.0 | 50 μM |
| I-10 | 7893 | 2.5 | 28.5 | 50 μM |
| I-11 | 8324 | 2.4 | 25.6 | 50 μM |
| I-12 | 6924 | 2.7 | 35.8 | 50 μM |

Data from various runs are presented below (IC50, nM unless indicated otherwise). Among other things, the present disclosure demonstrates that provided technologies can effectively neutralize SARS-Cov-2 viruses and reduce, inhibit or prevent infection by SARS-Cov-2 viruses.

| Agent | Run 1 | Run 2 | Run 3 | Run 4 | Run 5 | Run 6 |
|---|---|---|---|---|---|---|
| I-1 | | | 1.4 μM | 1.1 μM | | 0.68 nM |
| | | | | | | (potential |
| | | | | | | error) |
| I-2 | | | 5.6 μM | 1.6 μM | | 1,362 nM, 3,706 nM |
| I-28-1 | | | | <4.9 | | |
| I-28-2 | | | | | 264.5 | |
| I-28-3 | | | | | n = 1 31.8; | 2.7, 7.9 |
| | | | | | n = 2 > 100 | |
| I-26 | | | | inactive | <0.00018 | |
| I-27 | | | | 100% | <0.00018 | |
| | | | | suppression at | | |
| | | | | lowest conc 4.9 nM | | |
| I-29 | | | | | 2.14 | 0.027 |
| I-30 | | | | | 0.48 | 0.0276 |
| I-31 | | | | | 0.96 | 0.21 |
| I-32 | | | | | 13.1 | 0.44 |
| I-33 | | | | | 0.46 | 0.0069 |
| I-34 | | | | | 2.1 | 0.0229 |
| I-35 | | | | | 1.3 | 0.032 |
| I-36 | | | | | 2.1 | 0.0265; 0.069 |
| I-11 | | | | | <4.6 | 3.9 |
| I-6 | | | | | <4.6 | 1.7 |
| I-14 | | | | | <4.6 | 0.37 |
| I-18 | | | | | <4.6 | 0.25 |

### Example 5. Certain Technologies for Preparing Agents: I-24 and I-25

As appreciated by those skilled in the art, various chemical and biological procedures may be utilized in accordance with the present disclosure to provide agents and compositions of the present disclosure. Certain useful technologies are described below as examples.

In some embodiments, certain agents can be prepared by recombinant expression. For example, I-24 and I-25 were genetically engineered for expression in Freestyle293 cells. Each construct had expression plasmids made by Genescript in pCDNA3.1 and codon optimized for humans. The plasmids had the agents engineered with secretion signal from IgG-kappa and with C terminus tags of ggsEQKLISEEDLGSGGGGSLPETGGggsggsSHHHHHHHHHH (SEQ ID NO:90). I-24's amino acid sequence is DKEEILNKIYEIMRLLDELGNAEASMRVSDLILEFMKKGDERLLEEAERLLEEVER (SEQ ID NO:4). I-25's amino acid sequence is NDDELLMLVTDLVAEALLFAKDEEIKKRVFTLFELADKAYKNNDRDTLSKVVSELKELLERLQS (SEQ ID NO: 109).

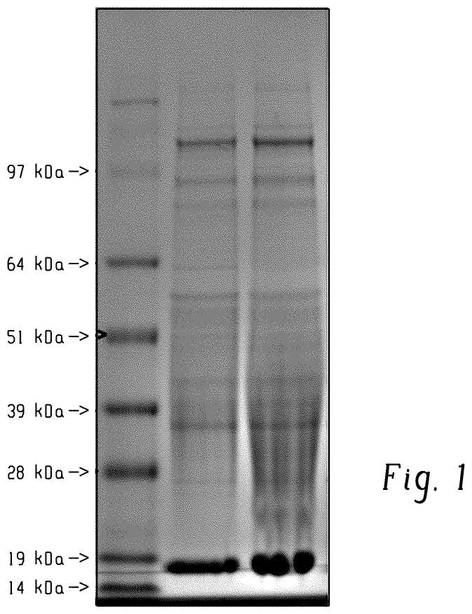

Transfection was in Freeestyle293 expression system following manufacture's protocol. Supernatant was harvested by spinning into a cell pellet and filtering supernatant with 0.45 uM filter system. NiNTA purification was used to purify the peptides from supernatant. Supernatant was incubated overnight in with HisPur NiNTA beads. The following day the supernatant and beads were pumped by parasitotic pump. The column was washed 10× with PBS and eluted with 0.25M imidazole in PBS. Certain results were presented in FIG. 1. Among other things, as demonstrated herein, 1-24 and 1-25 can bind to spike proteins or fragments thereof.

### Example 6. Certain Technologies for Preparing Agents: I-26, and I-27

Among other things, the present disclosure provides technologies for preparing various agents. For example, in some embodiments, the present disclosure provides technologies for preparing agents comprising antibody moieties and target binding moieties which are optionally linked by linker moieties (e.g., I-26 and I-27) from first agents comprising target binding moieties and first reactive groups which are optionally linked by first linker moieties (e.g., I-24, I-25, etc.) and second agents comprising antibody moieties and second reactive groups which are optionally linked by second linker moieties. In some embodiments, the present disclosure provides technologies for preparing second agents from antibody agents (e.g., Gamunex-C) and agents comprising antibody binding moieties and reactive groups which are optionally linked by linker moieties (e.g., compounds of formula R-I or salts thereof such as I-37).

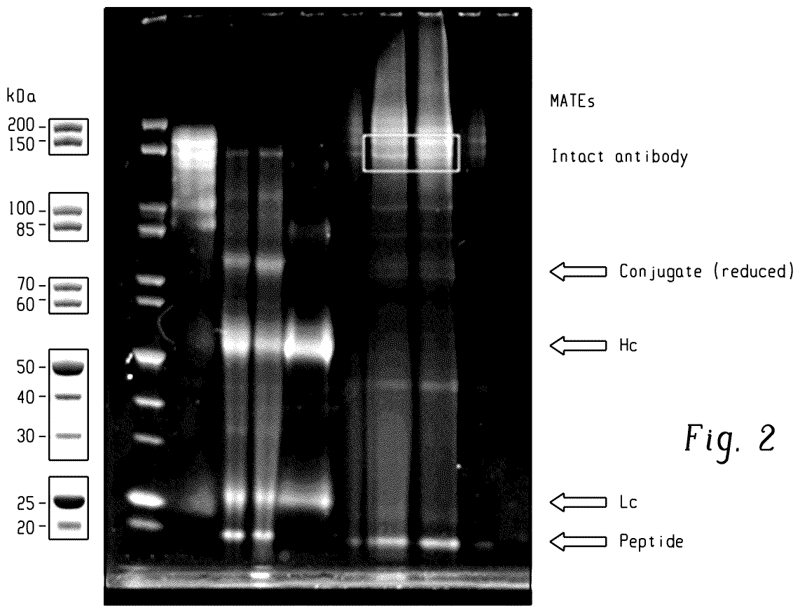

In some embodiments, antibody agents in Gamunex-C were conjugated with I-37 through chemically directed site specific conjugation technologies as described herein to provide I-38, a second agent composition wherein IVIG represents antibody moieties from Gamunex-C, and reactive groups are linked through a linker to lysine residues (through lysine side chains; in some embodiments, primarily K246 and/or K248 of an IgG1 heavy chain and amino acid residues corresponding thereto, K251 and/or K253 of an IgG2 heavy chain and amino acid residues corresponding thereto, and/or K239 and/or K241 of an IgG4 heavy chain and amino acid residues corresponding thereto). In some embodiments, conjugation conditions were incubating 2.5M equivalents of I-37 to Gamunex-C at 13 mg/mL in borate pH 8.2 buffer at room temperature for 16-22 hours. It was then followed by buffer exchanged into TBS pH 7.4 by Amicon spin concentrator, 30K MWCO. I-24 and I-25 were buffer exchanged into TBS pH 7.4 using Amicon spin concentrator, 3K MWCO. Sortase reaction was set up for overnight reaction between Gamunex-C modified with I-37 with I-24 and I-25, respectively. Sortase reactions also included 10 uM of CaCl₂ and sortase enzyme. Sortase conjugation incubated overnight shaking at 800 rpm. I-26 is sortase conjugation of I-24 and I-27 is sortase conjugation of I-25. Certain results were presented in FIG. 2.

### Example 7. Certain Technologies for Preparing Agents: I-28-1, I-28-2, and I-28-3

Among other things, the present disclosure provides technologies for preparing various agents. For example, in some embodiments, the present disclosure provides technologies for preparing agents comprising antibody moieties and target binding moieties which are optionally linked by linker moieties (e.g., I-28-1, I-28-2 and I-28-3) from first agents comprising target binding moieties and first reactive groups which are optionally linked by first linker moieties (e.g., I-3) and second agents comprising antibody moieties, such as antibody agents (e.g., Gamunex-C (I-28-1), Flebogamma (I-28-2), Gamunex-C (I-28-3), etc.).

In some embodiments, antibody agents, e.g., in Gamunex-C, Flebogamma, etc., were conjugated with I-3 through chemically directed site specific conjugation technologies as described herein to provide I-28-1, I-28-2, and I-28-3, second agent compositions wherein IVIG represents antibody moieties from Gamunex-C, and reactive groups are linked through a linker to lysine residues (through lysine side chains; in some embodiments, primarily K246 and/or K248 of an IgG1 heavy chain and amino acid residues corresponding thereto, K251 and/or K253 of an IgG2 heavy chain and amino acid residues corresponding thereto, and/or K239 and/or K241 of an IgG4 heavy chain and amino acid residues corresponding thereto). In some embodiments, conjugation conditions were incubating 2.5M equivalents of I-37 to Gamunex-C at 13 mg/mL in borate pH 8.2 buffer at room temperature for 16-22 hours. It was then followed by buffer exchanged into TBS pH 7.4 by Amicon spin concentrator, 30K MWCO. I-24 and I-25 were buffer exchanged into TBS pH 7.4 using Amicon spin concentrator, 3K MWCO. Sortase reaction was set up for overnight reaction between Gamunex-C modified with I-37 with I-24 and I-25, respectively. Sortase reactions also included 10 uM of CaCl₂ and sortase enzyme. Sortase conjugation incubated overnight shaking at 800 rpm. I-26 is sortase conjugation of I-24 and I-27 is sortase conjugation of I-25. Certain results were presented in FIG. 2. In some embodiments, Gammunex was buffer exchanged into borate buffer before conjugation. In some embodiments, conjugation conditions were incubating 2.5M equivalents of I-3 to IVIG (Gamunex-C or Flebogamma) at 20 mg/mL in borate pH 8.2 buffer at 25° C. for 16-22 hours. It was then followed by buffer exchanged into PBS by Amicon spin concentrator, 50K MWCO.

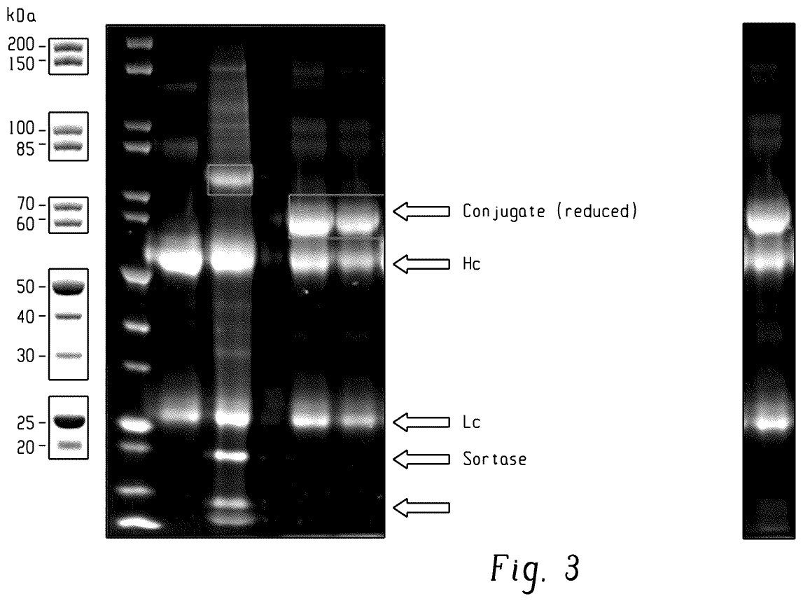

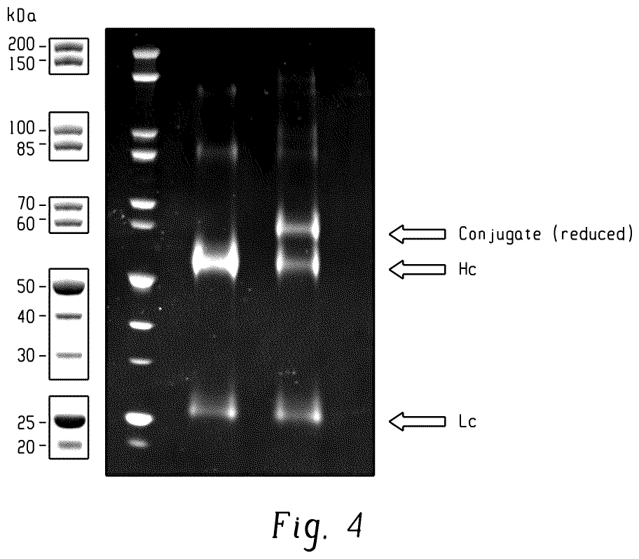

Certain results were presented in FIG. 3 and FIG. 4. In some embodiments, MATE product agents were cleaned up with 50 kDa concentrators. In some embodiments, follow-through was collected and concentrated using 3 kDa concentrators. In some embodiments, low molecular agents, which might contain unconjugated peptide moieties, might pass through 3 kDa filter. In some embodiments, some levels of losses, e.g., about 10% of MATE/antibody agents, was observed (see FIG. 3). Product compositions were further characterized and confirmed using other technologies, e.g., LCMS.

In some embodiments, instead of I-3, I-39 was utilized in reactions to provide I-40. Certain results were presented in FIG. 4.

In various embodiments, products from various sources of IVIG can provide the same or similar properties and/or activities.

### Example 8. Certain Technologies for Preparing Agents: I-1 and I-2

Various technologies including many peptide synthesis technologies can be utilized to prepare agents in accordance with the present disclosure, particularly those polypeptide agents or agents comprising polypeptide moieties. For example, in some embodiments, I-1 was prepared using the following procedure.

A solid phase peptide synthesis using standard Fmoc chemistry:
1) Resin preparation: DMF (10.0 mL) was added to the vessel containing Rink amide AM Resin (sub: 0.236 mmol/g, 0.1 mmol, 0.42 g) and swelled for 2 hrs.
2) Deprotection: 20% piperidine/DMF was added to the resin with N₂ bubbling for about 0.5 hr. The deprotection reaction was monitored by ninhydrin test, if it showed blue or other brownish red, the reaction was completed. The resin was then washed with DMF (20.0 mL)*5.
3) Coupling: Fmoc-amino acid solution in DMF (10.0 mL) was added to resin with N₂ bubbling, then activation buffer was added to the resin with N₂ bubbling for about 1 hr. The coupling reaction was monitored by ninhydrin test, if it showed blue, double coupling would be performed; if it showed colorless, the coupling was completed. The resin was then washed with DMF (20.0 mL)*5.
4) Step 2 to 3 was repeated for all other amino acids: (2-56 in Table below), double coupling was performed for amino acids Glu4, Glu9, Glu11, Glu12, Leu14, Asp17, Gly18, Lys19, Phe22, Leu24, Leu42, Arg43, Ile45, Glu46, Lys49, Glu53, Glu54, Lys55, and Asp56.
5) Acetylation: A solution of 10% Ac₂O/5% NMM/85% DMF (20.0 mL) was added to resin and the mixture was bubbled with N₂ for 20 mins. The coupling reaction was monitored by ninhydrin test, if it showed colorless, the coupling was completed. The resin was then washed with DMF (20.0 mL)*5, MeOH (20.0 mL)*5, and then dried under vacuum.

| # | Materials | Coupling reagents |
|---|---|---|
| 1 | Fmoc—Arg(Pbf)—OH (3.00 eg) | HATU (2.85 eq) and DIEA (6.00 eg) |
| 2 | Fmoc—Glu(OtBu)—OH (3.00 eg) | HATU (2.85 eg) and DIEA (6.00 eq) |
| 3 | Fmoc—Val—OH (3.00 eg) | HATU (2.85 eq) and DIEA (6.00 eg) |
| 4 | Fmoc—Glu(OtBu)—OH (3.00 eg) | HATU (2.85 eq) and DIEA (6.00 eg) |
| 5 | Fmoc—Glu(OtBu)—OH (3.00 eg) | HATU (2.85 eq) and DIEA (6.00 eg) |
| 6 | Fmoc—Leu—OH (3.00 eq) | HATU (2.85 eq) and DIEA (6.00 eg) |
| 7 | Fmoc—Leu—OH (3.00 eq) | HATU (2.85 eq) and DIEA (6.00 eq) |
| 8 | Fmoc—Arg(Pbf)—OH (3.00 eg) | HATU (2.85 eq) and DIEA (6.00 eg) |
| 9 | Fmoc—Glu(OtBu)—OH (3.00 eg) | HATU (2.85 eq) and DIEA (6.00 eg) |
| 10 | Fmoc—Ala—OH (3.00 eg) | HATU (2.85 eg) and DIEA (6.00 eq) |
| 11 | Fmoc—Glu(OtBu)—OH (3.00 eg) | HATU (2.85 eg) and DIEA (6.00 eg) |
| 12 | Fmoc—Glu(OtBu)—OH (3.00 eg) | HATU (2.85 eq) and DIEA (6.00 eg) |
| 13 | Fmoc—Leu—OH (6.00 eg) | HATU (5.70 eq) and DIEA (12.00 eq) |
| 14 | Fmoc—Leu—OH (6.00 eg) | HATU (5.70 eq) and DIEA (12.00 eg) |
| 15 | Fmoc—Arg(Pbf)—OH (6.00 eq) | HATU (5.70 eq) and DIEA (12.00 eg) |
| 16 | Fmoc—Glu(OtBu)—OH (6.00 eg) | HATU (5.70 eq) and DIEA (12.00 eq) |
| 17 | Fmoc—Asp(OtBu)—OH (6.00 eq) | HATU (5.70 eq) and DIEA (12.00 eq) |
| 18 | Fmoc—Gly—OH (6.00 eq) | HATU (5.70 eq) and DIEA (12.00 eg) |
| 19 | Fmoc—Lys(Boc)—OH (6.00 eg) | HATU (5.70 eq) and DIEA (12.00 eq) |
| 20 | Fmoc—Lys(Boc)—OH (6.00 eg) | HATU (5.70 eq) and DIEA (12.00 eg) |
| 21 | Fmoc—Met—OH (9.00 eg) | HATU (8.55 eq) and DIEA (18.00 eg) |
| 22 | Fmoc—Phe—OH (9.00 eg) | HATU (8.55 eq) and DIEA (18.00 eq) |
| 23 | Fmoc—Glu(tBu)—OH (9.00 eg) | HATU (8.55 eq) and DIEA (18.00 eg) |
| 24 | Fmoc—Leu—OH (9.00 eq) | HATU (8.55 eq) and DIEA (18.00 eq) |
| 25 | Fmoc—Ile—OH (9.00 eg) | HATU (8.55 eq) and DIEA (18.00 eq) |
| 26 | Fmoc—Leu—OH (9.00 eg) | HATU (8.55 eq) and DIEA (18.00 eg) |
| 27 | Fmoc—Asp(OtBu)—OH (9.00 eg) | HATU (8.55 eq) and DIEA (18.00 eg) |
| 28 | Fmoc—Ser(tBu)—OH (9.00 eg) | HATU (8.55 eq) and DIEA (18.00 eq) |
| 29 | Fmoc—Val—OH (9.00 eg) | HATU (8.55 eq) and DIEA (18.00 eq) |
| 30 | Fmoc—Arg(Pbf)—OH (9.00 eg) | HATU (8.55 eq) and DIEA (18.00 eg) |
| 31 | Fmoc—Met—OH (9.00 eq) | HATU (8.55 eq) and DIEA (18.00 eq) |
| 32 | Fmoc—Ser(tBu)—OH (9.00 eg) | HATU (8.55 eg) and DIEA (18.00 eq) |
| 33 | Fmoc—Ala—OH (9.00 eq) | HATU (8.55 eq) and DIEA (18.00 eq) |
| 34 | Fmoc—Glu(OtBu)—OH (9.00 eg) | HATU (8.55 eq) and DIEA (18.00 eg) |
| 35 | Fmoc—Ala—OH (9.00 eg) | HATU (8.55 eq) and DIEA (18.00 eg) |
| 36 | Fmoc—Asn(Trt)—OH (9.00 eg) | HATU (8.55 eq) and DIEA (18.00 eq) |
| 37 | Fmoc—Gly—O—H (9.00 eg) | HATU (8.55 eq) and DIEA (18.00 eq) |
| 38 | Fmoc—Leu—OH (9.00 eg) | HATU (8.55 eq) and DIEA (18.00 eg) |
| 39 | Fmoc—Glu(OtBu)—OH (9.00 eg) | HATU (8.55 eq) and DIEA (18.00 eq) |
| 40 | Fmoc—Asp(OtBu)—OH (9.00 eq) | HATU (8.55 eq) and DIEA (18.00 eq) |
| 41 | Fmoc—Leu—OH (9.00 eg) | HATU (8.55 eq) and DIEA (18.00 eg) |
| 42 | Fmoc—Leu—OH (9.00 eg) | HATU (8.55 eq) and DIEA (18.00 eq) |
| 43 | Fmoc—Arg(Pbf)—OH (9.00 eq) | HATU (8.55 eq) and DIEA (18.00 eg) |
| 44 | Fmoc—Met—OH (9.00 eq) | HATU (8.55 eq) and DIEA (18.00 eg) |
| 45 | Fmoc—Ile—OH (9.00 eg) | HATU (8.55 eq) and DIEA (18.00 eg) |
| 46 | Fmoc—Glu(OtBu)—OH (9.00 eq) | HATU (8.55 eg) and DIEA (18.00 eg) |
| 47 | Fmoc—Tyr(tBu)—OH (9.00 eq) | HATU (8.55 eq) and DIEA (18.00 eq) |
| 48 | Fmoc—Ile—OH (9.00 eg) | HATU (8.55 eg) and DIEA (18.00 eq) |
| 49 | Fmoc—Lys(Boc)—OH (9.00 eg) | HATU (8.55 eq) and DIEA (18.00 eq) |
| 50 | Fmoc—Asn(Trt)—OH (9.00 eg) | HATU (8.55 eq) and DIEA (18.00 eq) |
| 51 | Fmoc—Leu—OH (9.00 eq) | HATU (8.55 eq) and DIEA (18.00 eq) |
| 52 | Fmoc—Ile—OH (9.00 eg) | HATU (8.55 eq) and DIEA (18.00 eq) |
| 53 | Fmoc—Glu(OtBu)—OH (9.00 eq) | HATU (8.55 eq) and DIEA (18.00 eg) |
| 54 | Fmoc—Glu(OtBu)—OH (9.00 eg) | HATU (8.55 eq) and DIEA (18.00 eq) |
| 55 | Fmoc—Lys(Boc)—OH (9.00 eg) | HATU (8.55 eq) and DIEA (18.00 eq) |
| 56 | Fmoc—Asp(OtBu)—OH (9.00 eq) | HATU (8.55 eq) and DIEA (18.00 eq) |
| 57 | Ac₂O | Ac₂O/NMM/DMF (10/5/85, 20.0 mL) |

### Peptide Cleavage and Purification:

1) Cleavage buffer (95% TFA/2.5% TIS/2.5% H₂O, 20 mL) was added to the flask containing the side chain protected peptide resin at room temperature and stirred for 2 hrs.
2) The filtrate was collected.
3) Peptide was precipitated with cold isopropyl ether and centrifuged (3 mins at 3000 rpm).
4) Isopropyl ether washed two additional times.
5) The crude peptide was dried under vacuum 2 hrs.
6) The crude peptide was purified by Prep_HPLC (A: 0.075% TFA in H₂O, B: ACN), followed by lyophilization to give compound I-1 (36.0 mg, 91.0% purity, 5.370 yield, TFA salt) as a white solid.

### Purification Conditions:

| | Separation condition |
|---|---|
| Dissolution | Dissolve in ACN/H₂O |
| condition | |
| Instrument | GX-281 |
| Mobile Phase | A: H₂O (TFA 0.075%) |
| | B: CH₃CN |
| Gradient | 30-60%-51 min. Retention time: 32 min |
| Column | Luna 100*25 mm, C18, 10 um, 100Å + Gemini ® |
| | 150*30 mm, C18, 5 um, 110Å |
| Flow Rate | 20 mL/min |
| Wavelength | 220/254 nm |
| Oven Temp. | Room temperature |

I-2 was prepared using similar peptide synthesis technologies.

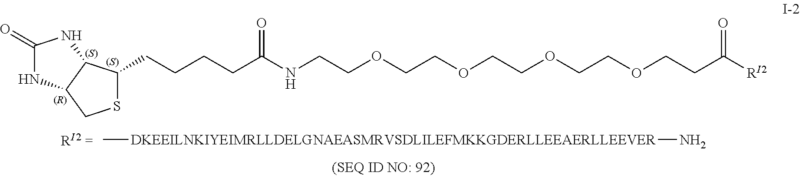

For one preparation, a purity of about 91% was observed for I-1. For one preparation, a purity of about 91% was observed for I-2. Expected masses were observed in MS for both compounds.

### Example 9. Certain Technologies for Preparing Agents: I-3

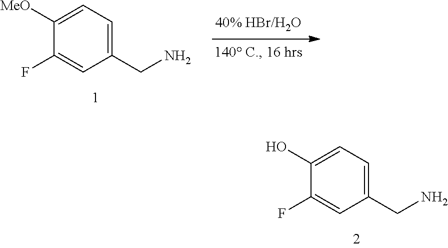 [SMILES: COc1ccc(CN)cc1F ; NCc1ccc(O)c(F)c1]

A mixture of compound 1 (10 g, 64.45 mmol) in HBr/H₂O (40% HBr, 300 mL in total) was stirred at 140° C. for 16 hrs. The solvent was removed at 70° C. under reduced pressure, the residue was triturated in MeCN (50 mL) for 10 mins. After filtered, the solid was dried under lyophilization to get compound 2 (13.0 g, 58.5 mmol, 90.8% yield, HBr salt) as a brown solid. ¹H NMR: (400 MHz DMSO-d₆) δ ppm 10.04 (s, 1H) 8.18 (s, 3H) 7.32 (dd, J=12.17, 1.88 Hz, 1H) 7.11 (dd, J=8.28, 1.51 Hz, 1H) 6.96-7.03 (m, 1H) 3.93 (q, J=5.52 Hz, 2H).

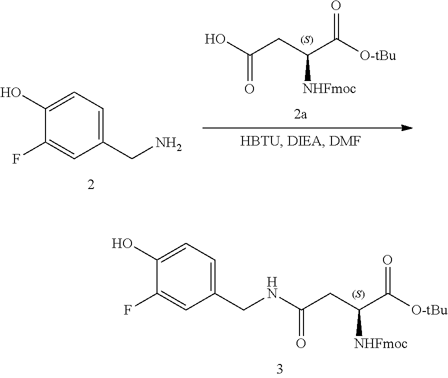

To a mixture of compound 2 (13.0 g, 58.5 mmol, 1 eq, HBr), compound 2a (24.1 g, 58.5 mmol, 1 eq), DIEA (3.78 g, 29.2 mmol, 5.10 mL, 0.5 eq) and HOBt (11.87 g, 87.8 mmol, 1.5 eq) in DMF (200 mL) was added EDCI (12.35 g, 64.4 mmol, 1.1 eq) at 15° C. The mixture was stirred at 15° C. for 3 hr. The mixture was dropwise added to 0.5 M HCl (cold, 1 L) and white solid was precipitated. After filtering, the solid was dried under lyophilization to afford compound 3 (31 g, crude) as a white solid.

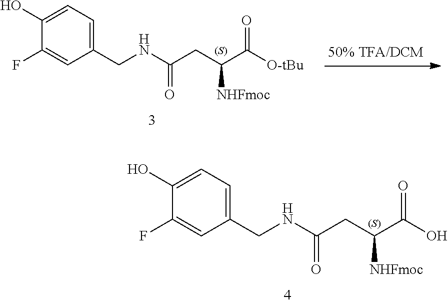

A mixture of compound 3 (30 g, 56.12 mmol, 1.0 eq) in TFA (300 mL) and DCM (300 mL) was stirred at 15° C. for 0.5 hr. The solvent was removed under reduced pressure. The residue was purified by flash C18 (ISCO®; 120 g SepaFlash® C18 Flash Column, Eluent of 0˜90% MeCN/H₂O gradient @ 75 mL/min) directly to get compound 4 (18 g, 37.6 mmol, 67.0% yield) as a white solid. Expected mass was observed in MS.

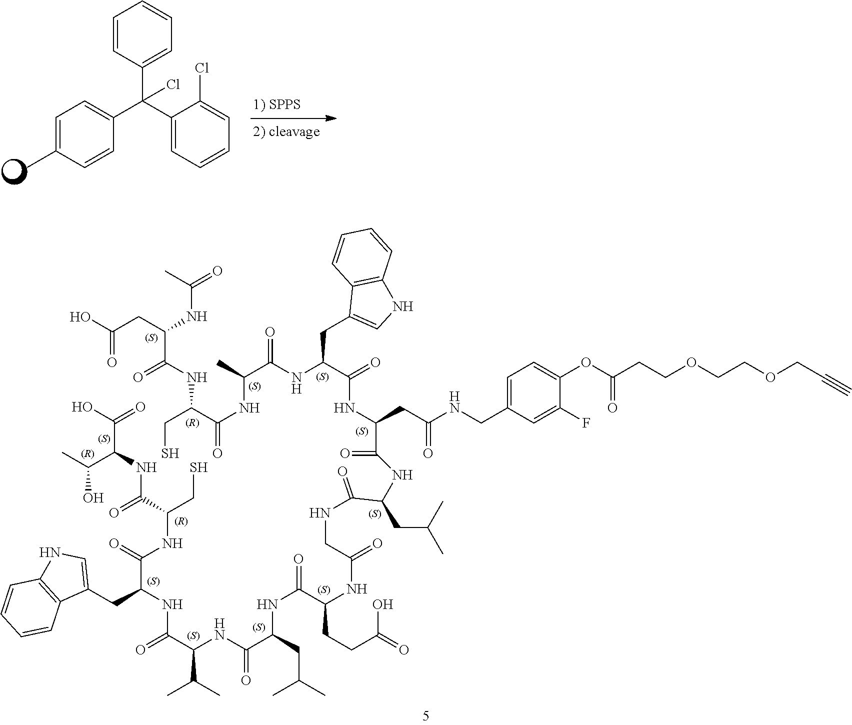

### Peptide was Synthesized Using Standard Fmoc Chemistry:

1) Resin preparation: To the vessel containing CTC Resin (3.0 mmol, 3.0 g, 1.00 mmol/g) and Fmoc-Thr(tBu)-OH (1.19 g, 3.0 mmol, 1.00 eq) in DCM (30 mL) was added DIEA (4.00 eq) dropwise and mixed for 2 hrs with N₂ bubbling at 15° C. Then MeOH (3.0 mL) was added and bubbled with N₂ for another 30 mins. The resin was washed with DMF (60 mL)*5, followed by adding 20% piperidine in DMF (60 mL) and bubbled with N₂ for 30 mins at 15° C. for Fmoc deprotection. The mixture was filtered and the resin was washed with DMF (60 mL)*5 before proceeding to next step.
2) Coupling: A solution of Fmoc-Cys(Trt)-OH (5.25 g, 3.00 eq), HBTU (3.24 g, 2.85 eq) in DMF (30 mL) was added to the resin with N₂ bubbling. Then DIEA (6.00 eq) was added to the mixture dropwise and bubbled with N₂ for 30 mins at 15° C. The coupling reaction was monitored by ninhydrin test, if it showed colorless, the coupling was completed. The resin was then washed with DMF (60 mL)*5.
3) De-protection: 20% piperidine in DMF (60 mL) was added to the resin and the mixture was bubbled with N₂ for 30 mins at 15° C. The deprotection reaction was monitored by ninhydrin test, if it showed blue or other brownish red, the reaction was completed. The resin was then washed with DMF (60 mL)*5.
4) Step 2 and 3 were repeated for amino acids: (2-8 in table below).
5) Coupling: A solution of compound 4 (2.87 g, 2.00 eq), DIC (0.76 g, 2.00 eq) and HOBt (0.82 g, 2.00 eq) in DMF (30 mL) was added to the resin with N₂ bubbling for 60 mins at 15° C. The coupling reaction was monitored by ninhydrin test, if it showed colorless, the coupling was completed. The resin was then washed with DMF (60 mL)*5.
6) Step 3 was repeated for Fmoc deprotection.
7) Step 5 and 6 were repeated for amino acids (10-13 in table below).
8) Acetylation: A solution of 10% Ac₂O/5% NMM/85% DMF (60 mL) was added to resin and the mixture was bubbled with N₂ for 20 mins. The acetylation reaction was monitored by ninhydrin test, if it showed colorless, the coupling was completed. The resin was then washed with DMF (60 mL)*5.
9) 3% hydrazine in DMF (60 mL) was added to resin with N₂ bubbling for 15 min to liberate hydroxyl group on compound 4, the resin was then washed with DMF (60 mL)*5.
10) Coupling for the last position: A solution of 3-(2-(prop-2-yn-1-yloxy)ethoxy)propanoic acid (1.54 g, 9.0 mmol, 3.00 eq), DIC (1.13 g, 3.00 eq), HOBt (1.23 g, 3.00 eq) and DMAP (1.10 g, 3.00 eq) was added to resin and the mixture was bubbled with N₂ for 36 hrs. The coupling reaction was monitored by LCMS after a mini-cleavage, almost 50% was desired MS. The resin was then washed with DMF (60 mL)*5, MeOH (60 mL)*5, and then dried under vacuum.

| | | |
|---|---|---|
| # | Materials | Coupling reagents |

| | | | |
|---|---|---|---|
| 1 | Fmoc-Thr(tBu)—OH | (1.00 eq) | DIEA (4.00 eg) |
| 2 | Fmoc-Cys(Trt)-OH | (3.00 eg) | HBTU (2.85 eq) and DIEA (6.00 eq) |
| 3 | Fmoc-Trp-OH | (3.00 eq) | HBTU (2.85 eq) and DIEA (6.00 eq) |
| 4 | Fmoc-Val-OH | (3.00 eq) | HBTU (2.85 eq) and DIEA (6.00 eq) |
| 5 | Fmoc-Leu-OH | (3.00 eq) | HBTU (2.85 eq) and DIEA (6.00 eq) |
| 6 | Fmoc-Glu(OtBu)—OH | (3.00 eq) | HBTU (2.85 eq) and DIEA (6.00 eq) |
| 7 | Fmoc-Gly-OH | (3.00 eg) | HBTU (2.85 eq) and DIEA (6.00 eg) |
| 8 | Fmoc-Leu-OH | (3.00 eq) | HBTU (2.85 eq) and DIEA (6.00 eg) |
| 9 | compound 4 | (2.00 eq) | DIC (2.00 eq) and HOBt (2.00 eq) |
| 10 | Fmoc-Trp-OH | (3.00 eg) | DIC (3.00 eq) and HOBt (3.00 eq) |
| 11 | Fmoc-Ala-OH | (3.00 eg) | DIC (3.00 eq) and HOBt (3.00 eg) |
| 12 | Fmoc-Cys(Trt)-OH | (3.00 eg) | DIC (3.00 eq) and HOBt (3.00 eg) |
| 13 | Fmoc-Asp(OtBu)—OH | (3.00 eg) | DIC (3.00 eg) and HOBt (3.00 eq) |

| | | |
|---|---|---|
| 14 | Acetylation | Ac₂O/NMM/DMF (10/5/85, 60 mL) |

| | | | |
|---|---|---|---|
| 15 | 3-(2-(prop-2-yn-1-yloxy) | (3.00 eg) | DIC (3.00 eq), HOBt (3.00 eq) |
| | ethoxy)propanoic acid | | and DMAP (3.00 eq) |

### Peptide Cleavage and Purification:

1) Cleavage buffer (95% TFA/2.5% Tis/2.5% H₂O, 60 mL) was added to the flask containing the side chain protected peptide resin at room temperature and stirred for 1 hr.
2) The filtrate was collected.
3) The peptide was precipitated with cold isopropyl ether (300 mL) and centrifuged (3 mins at 3000 rpm).
4) Isopropyl ether washed two additional times, and the crude peptide was dried under vacuum for 2 hrs.
5) Compound 5 (4.2 g, crude) was obtained as a white solid.

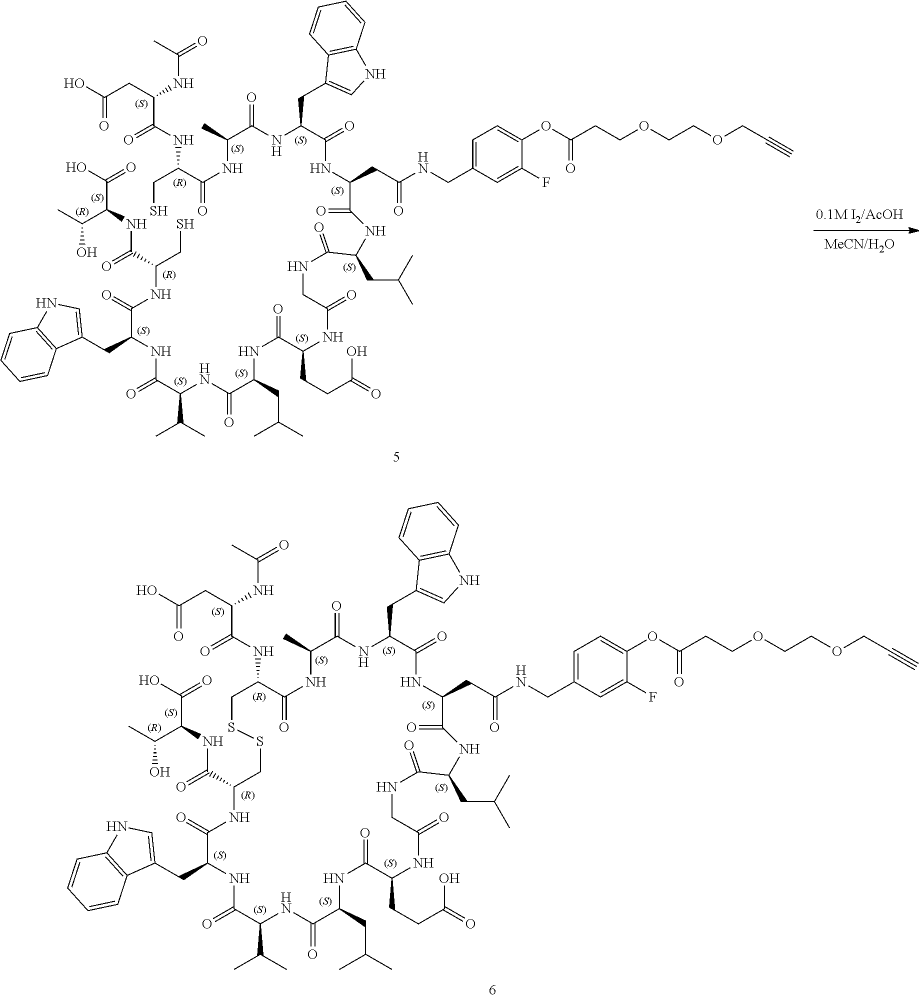

To a mixture of compound 5 (4.2 g, crude) in MeCN/H₂O (1/1, 3.0 L) was added 0.1 M I₂/HOAc dropwise until the yellow color persisted, then the mixture was stirred at 15° C. for 5 mins. The mixture was quenched with 0.1 M Na₂S₂O₃ dropwise until yellow color disappeared. The mixture was lyophilized to give the crude powder. The crude peptide was purified by prep-HPLC (acid condition, TFA), followed by lyophilization to afford compound 6 (500 mg, 90.1% purity, 9.11% yield) as a white solid. Expected mass was observed in MS.

In some embodiments, compound 6 is utilized as a compound of formula R-I (SEQ ID NO:92).

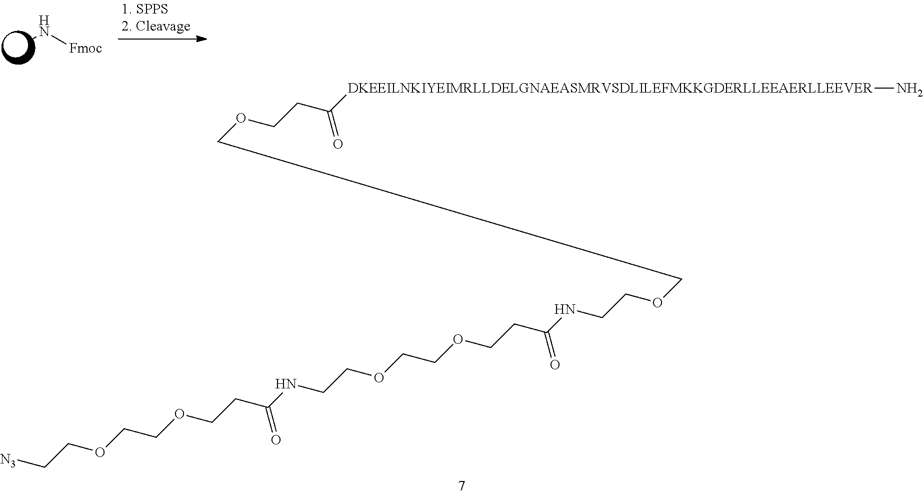

### Peptide was Synthesized Using Standard Fmoc Chemistry.

1) Resin preparation: DMF (10.0 mL) was added to the vessel containing Rink amide AM Resin (sub: 0.236 mmol/g, 0.1 mmol, 0.42 g) and swelled for 2 hrs.
2) Deprotection: 20% piperidine/DMF was added to the resin with N₂ bubbling for about 0.5 hr. The deprotection reaction was monitored by ninhydrin test, if it showed blue or other brownish red, the reaction was completed. The resin was then washed with DMF (20.0 mL)*5.
3) Coupling: Fmoc-amino acid solution in DMF (10.0 mL) was added to resin with N₂ bubbling, then activation buffer was added to the resin with N₂ bubbling for about 1 hr. The coupling reaction was monitored by ninhydrin test, if it showed blue, double coupling would be performed; if it showed colorless, the coupling was completed. The resin was then washed with DMF (20.0 mL)*5.
4) Repeat step 2 to 3 for all other amino acids: (2-56 in table below). Double coupling was performed for amino acids (Glu4, Glu9, Glu11, Glu12, Leu14, Asp17, Gly18, Lys19, Phe22, Leu24, Leu42, Arg43, Ile45, Glu46, Lys49, Glu53, Glu54, Lys55, and Asp56 in table below).

| # | Materials | | Coupling reagents |
|---|---|---|---|
| 1 | Fmoc-Arg(Pbf)-OH | (3.00 eq) | HATU (2.85 eq) and DIEA (6.00 eq) |
| 2 | Fmoc-Glu(OtBu)—OH | (3.00 eg) | HATU (2.85 eq) and DIEA (6.00 eq) |
| 3 | Fmoc-Val-OH | (3.00 eg) | HATU (2.85 eq) and DIEA (6.00 eg) |
| 4 | Fmoc-Glu(OtBu)—OH | (3.00 eq) | HATU (2.85 eq) and DIEA (6.00 eq) |
| 5 | Fmoc-Glu(OtBu)—OH | (3.00 eg) | HATU (2.85 eq) and DIEA (6.00 eq) |
| 6 | Fmoc-Leu-OH | (3.00 eq) | HATU (2.85 eq) and DIEA (6.00 eq) |
| 7 | Fmoc-Leu-OH | (3.00 eg) | HATU (2.85 eq) and DIEA (6.00 eq) |
| 8 | Fmoc-Arg(Pbf)-OH | (3.00 eg) | HATU (2.85 eq) and DIEA (6.00 eq) |
| 9 | Fmoc-Glu(OtBu)—OH | (3.00 eg) | HATU (2.85 eq) and DIEA (6.00 eg) |
| 10 | Fmoc-Ala-OH | (3.00 eg) | HATU (2.85 eq) and DIEA (6.00 eq) |
| 11 | Fmoc-Glu(OtBu)—OH | (3.00 eg) | HATU (2.85 eq) and DIEA (6.00 eg) |
| 12 | Fmoc-Glu(OtBu)—OH | (3.00 eq) | HATU (2.85 eq) and DIEA (6.00 eq) |
| 13 | Fmoc-Leu-OH | (6.00 eg) | HATU (5.70 eq) and DIEA (12.00 eq) |
| 14 | Fmoc-Leu-OH | (6.00 eg) | HATU (5.70 eg) and DIEA (12.00 eq) |
| 15 | Fmoc-Arg(Pbf)-OH | (6.00 eg) | HATU (5.70 eq) and DIEA (12.00 eg) |
| 16 | Fmoc-Glu(OtBu)—OH | (6.00 eg) | HATU (5.70 eq) and DIEA (12.00 eq) |
| 17 | Fmoc-Asp(OtBu)—OH | (6.00 eg) | HATU (5.70 eq) and DIEA (12.00 eq) |
| 18 | Fmoc-Gly-OH | (6.00 eg) | HATU (5.70 eq) and DIEA (12.00 eg) |
| 19 | Fmoc-Lys(Boc)-OH | (6.00 eg) | HATU (5.70 eq) and DIEA (12.00 eg) |
| 20 | Fmoc-Lys(Boc)-OH | (6.00 eq) | HATU (5.70 eq) and DIEA (12.00 eg) |
| 21 | Fmoc-Met-OH | (9.00 eg) | HATU (8.55 eq) and DIEA (18.00 eg) |
| 22 | Fmoc-Phe-OH | (9.00 eg) | HATU (8.55 eq) and DIEA (18.00 eq) |
| 23 | Fmoc-Glu(tBu)—OH | (9.00 eg) | HATU (8.55 eq) and DIEA (18.00 eg) |
| 24 | Fmoc-Leu-OH | (9.00 eg) | HATU (8.55 eq) and DIEA (18.00 eq) |
| 25 | Fmoc-Ile-OH | (9.00 eq) | HATU (8.55 eq) and DIEA (18.00 eq) |
| 26 | Fmoc-Leu-OH | (9.00 eq) | HATU (8.55 eq) and DIEA (18.00 eg) |
| 27 | Fmoc-Asp(OtBu)—OH | (9.00 eq) | HATU (8.55 eq) and DIEA (18.00 eg) |
| 28 | Fmoc-Ser(tBu)—OH | (9.00 eg) | HATU (8.55 eq) and DIEA (18.00 eg) |
| 29 | Fmoc-Val-OH | (9.00 eg) | HATU (8.55 eq) and DIEA (18.00 eg) |
| 30 | Fmoc-Arg(Pbf)-OH | (9.00 eg) | HATU (8.55 eq) and DIEA (18.00 eg) |
| 31 | Fmoc-Met-OH | (9.00 eg) | HATU (8.55 eq) and DIEA (18.00 eg) |
| 32 | Fmoc-Ser(Bu)—OH | (9.00 eg) | HATU (8.55 eq) and DIEA (18.00 eq) |
| 33 | Fmoc-Ala-OH | (9.00 eq) | HATU (8.55 eq) and DIEA (18.00 eg) |
| 34 | Fmoc-Glu(OtBu)—OH | (9.00 eq) | HATU (8.55 eq) and DIEA (18.00 eg) |
| 35 | Fmoc-Ala-OH | (9.00 eg) | HATU (8.55 eq) and DIEA (18.00 eq) |
| 36 | Fmoc-Asn(Trt)-OH | (9.00 eg) | HATU (8.55 eq) and DIEA (18.00 eg) |
| 37 | Fmoc-Gly-OH | (9.00 eg) | HATU (8.55 eq) and DIEA (18.00 eq) |
| 38 | Fmoc-Leu-OH | (9.00 eg) | HATU (8.55 eq) and DIEA (18.00 eg) |
| 39 | Fmoc-Glu(OtBu)—OH | (9.00 eq) | HATU (8.55 eq) and DIEA (18.00 eg) |
| 40 | Fmoc-Asp(OtBu)—OH | (9.00 eq) | HATU (8.55 eq) and DIEA (18.00 eg) |
| 41 | Fmoc-Leu-OH | (9.00 eg) | HATU (8.55 eq) and DIEA (18.00 eg) |
| 42 | Fmoc-Leu-OH | (9.00 eq) | HATU (8.55 eq) and DIEA (18.00 eg) |
| 43 | Fmoc-Arg(Pbf)-OH | (9.00 eg) | HATU (8.55 eq) and DIEA (18.00 eg) |
| 44 | Fmoc-Met-OH | (9.00 eq) | HATU (8.55 eq) and DIEA (18.00 eq) |
| 45 | Fmoc-Ile-OH | (9.00 eg) | HATU (8.55 eq) and DIEA (18.00 eg) |
| 46 | Fmoc-Glu(OtBu)—OH | (9.00 eg) | HATU (8.55 eq) and DIEA (18.00 eg) |
| 47 | Fmoc-Tyr(tBu)—OH | (9.00 eg) | HATU (8.55 eq) and DIEA (18.00 eg) |
| 48 | Fmoc-Ile-OH | (9.00 eg) | HATU (8.55 eq) and DIEA (18.00 eg) |
| 49 | Fmoc-Lys(Boc)-OH | (9.00 eq) | HATU (8.55 eq) and DIEA (18.00 eg) |
| 50 | Fmoc-Asn(Trt)-OH | (9.00 eg) | HATU (8.55 eq) and DIEA (18.00 eg) |
| 51 | Fmoc-Leu-OH | (9.00 eg) | HATU (8.55 eq) and DIEA (18.00 eq) |
| 52 | Fmoc-Ile-OH | (9.00 eq) | HATU (8.55 eq) and DIEA (18.00 eq) |
| 53 | Fmoc-Glu(OtBu)—OH | (9.00 eg) | HATU (8.55 eq) and DIEA (18.00 eg) |
| 54 | Fmoc-Glu(OtBu)—OH | (9.00 eg) | HATU (8.55 eq) and DIEA (18.00 eg) |
| 55 | Fmoc-Lys(Boc)-OH | (9.00 eg) | HATU (8.55 eq) and DIEA (18.00 eg) |
| 56 | Fmoc-Asp(OtBu)—OH | (9.00 eg) | HATU (8.55 eq) and DIEA (18.00 eq) |
| 57 | Fmoc-PEG₂-CH₂CH₂COOH | (3.00 eg) | HATU (2.85 eq) and DIEA (6.00 eg) |
| 58 | Fmoc-PEG₂-CH₂CH₂COOH | (3.00 eq) | HATU (2.85 eq) and DIEA (6.00 eg) |
| 59 | 3-(2-(2-azidoethoxy)ethoxy)propanoic acid | (3.00 eq) | HATU (2.85 eq) and DIEA (6.00 eq) |

### Peptide Cleavage and Purification:

1) Cleavage buffer (95% TFA/2.5% TIS/2.5% H₂O, 20 mL) was added to the flask containing the side chain protected peptide resin at room temperature and stirred for 2 hrs.
2) The filtrate was collected.
3) The peptide was precipitated with cold isopropyl ether and centrifuged (3 mins at 3000 rpm).
4) Isopropyl ether washed two additional times.
5) The crude peptide was dried under vacuum 2 hrs.
6) The crude peptide was purified by Prep_HPLC (A: 0.075% TFA in H₂O, B: ACN), followed by lyophilization to afford compound 7 (26.0 mg, 93.0% purity, 3.63% yield, TFA salt) as a white solid. Expected mass was observed in MS.

In some embodiments, compound 7 is utilized as an agent comprising a target binding moiety and a reactive group (e.g., N₃).

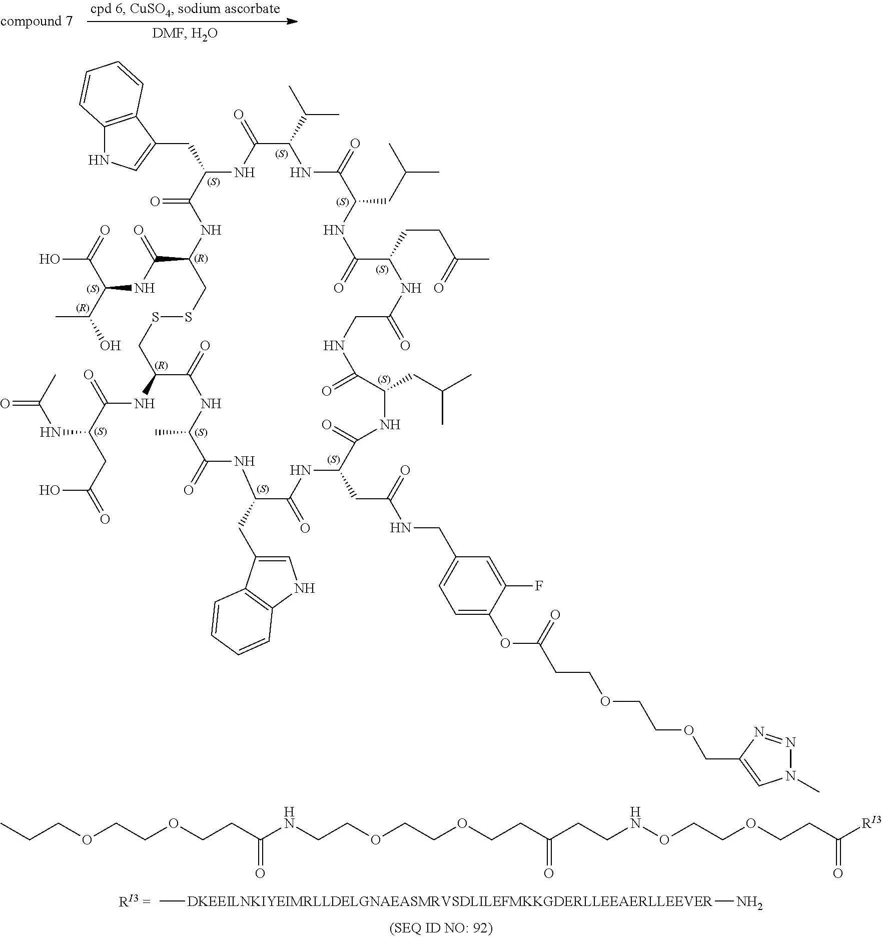

A mixture of compound 6 (6.63 mg, 3.63 umol, 1.00 eq), compound 7 (26.0 mg, 3.63 umol, 1.00 eq), CuSO₄ (0.4 M, 9.07 uL, 1.00 eq) and sodium ascorbate (0.4 M, 36.27 uL, 4.00 eq) in DMF (0.3 mL) was stirred at 15° C. for 1 hr under N₂ atmosphere. The mixture was purified by prep-HPLC (acid condition, TFA) to afford compound I-3 (1.8 mg, 85.1% purity, 4.69% yield, TFA salt) as a white solid. Expected mass was observed. Among other things, compound 1-3 may be utilized as an agent comprising a target binding moiety, a reactive group and optionally a linker moiety linking the two.

### Purification Conditions:

Separation condition

| Dissolution | Dissolve in ACN/H₂O | |
|---|---|---|
| condition | | |
| Instrument | GX-281 | |
| Mobile | A: | H₂O (TFA 0.075%) |
| Phase | B: | CH₃CN |
| Gradient | 30-60%-51 min. Retention time: 35 min | |
| Column | Luna 100*25 mm, C18, 10 um, 100 Å + | |
| | Gemini ® 150*30 mm, C18, 5 um, 110 Å | |
| Flow Rate | 20 | mL/min |
| Wavelength | 220/254 | nm |

| | |
|---|---|
| Oven Temp. | Room temperature |

### Example 10. Certain Technologies for Preparing Agents: I-4, I-5 and I-6

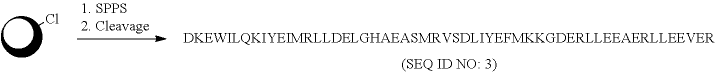

### Peptide was Synthesized Using Standard Fmoc Chemistry.

1) Resin preparation: To the vessel containing CTC Resin (0.10 mmol, 0.13 g, 0.78 mmol/g) and Fmoc-Arg(Pbf)-OH (64.91 mg, 0.10 mmol, 1.00 eq) in DCM (2.0 mL) was dropwise added DIEA (4.00 eq) and mixed for 2 hrs with N₂ bubbling at 15° C. Then MeOH (0.10 mL) was added and bubbled with N₂ for another 30 mins. The resin was washed with DMF (10.0 mL)*5, followed by adding 20% piperidine in DMF (10.0 mL) and bubbled with N₂ for 15 mins at 15° C. for Fmoc deprotection. The mixture was filtered and the resin was washed with DMF (10.0 mL)*5 before proceeding to next step.
2) Coupling: A solution of Fmoc-Glu(OtBu)-OH (0.13 g, 3.00 eq), HBTU (0.11 g, 2.85 eq) in DMF (2.0 mL) was added to the resin with N₂ bubbling. Then DIEA (6.00 eq) was added to the mixture dropwise and bubbled with N₂ for 30 mins at 15° C. The coupling reaction was monitored by ninhydrin test, if the resin showed blue or brownish red, double coupling should be performed; if it showed colorless, the coupling was completed. The resin was then washed with DMF (10.0 mL)*5.
3) Deprotection: 20% piperidine in DMF (10.0 mL) was added to the resin and the mixture was bubbled with N₂ for 15 mins at 15° C. The De-protection reaction was monitored by ninhydrin test, if it showed blue or brownish red, the reaction was completed. The resin was then washed with DMF (10.0 mL)*5.
4) Step 2 to 3 was repeated for all other amino acids: (2-56 in table below), double coupling was performed for amino acids (Arg8, Glu12, Leu14, Asp17, Phe22, Arg43, Ile45, Glu53, and Glu54 in table below).
5) After de-Fmoc of the last amino acid, the resin was washed with DMF (10.0 mL)*5, MeOH (10.0 mL)*5, and dried under vacuum.

| | | |
|---|---|---|
| # | Materials | Coupling reagents |

| | | | |
|---|---|---|---|
| 1 | Fmoc-Arg(Pbf)-OH | (1.00 eg) | DIEA (4.00 eg) |
| 2 | Fmoc-Glu(OtBu)—OH | (3.00 eg) | HBTU (2.85 eg) and DIEA (6.00 eg) |
| 3 | Fmoc-Val-OH | (3.00 eg) | HBTU (2.85 eq) and DIEA (6.00 eg) |
| 4 | Fmoc-Glu(OtBu)—OH | (3.00 eg) | HBTU (2.85 eq) and DIEA (6.00 eg) |
| 5 | Fmoc-Glu(OtBu)-OH | (3.00 eq) | HBTU (2.85 eq) and DIEA (6.00 eq) |
| 6 | Fmoc-Leu-OH | (3.00 eq) | HBTU (2.85 eq) and DIEA (6.00 eg) |
| 7 | Fmoc-Leu-OH | (3.00 eg) | HBTU (2.85 eq) and DIEA (6.00 eg) |
| 8 | Fmoc-Arg(Pbf)-OH | (3.00 eq) | HBTU (2.85 eq) and DIEA (6.00 eg) |
| 9 | Fmoc-Glu(OtBu)—OH | (6.00 eg) | HATU (5.70 eq) and DIEA (12.00 eq) |
| 10 | Fmoc-Ala-OH | (6.00 eq) | HATU (5.70 eq) and DIEA (12.00 eq) |
| 11 | Fmoc-Glu(OtBu)—OH | (6.00 eq) | HATU (5.70 eq) and DIEA (12.00 eq) |
| 12 | Fmoc-Glu(OtBu)—OH | (6.00 eq) | HATU (5.70 eq) and DIEA (12.00 eq) |
| 13 | Fmoc-Leu-OH | (6.00 eg) | HATU (5.70 eq) and DIEA (12.00 eg) |
| 14 | Fmoc-Leu-OH | (6.00 eq) | HATU (5.70 eq) and DIEA (12.00 eq) |
| 15 | Fmoc-Arg(Pbf)-OH | (6.00 eq) | HATU (5.70 eq) and DIEA (12.00 eq) |
| 16 | Fmoc-Glu(OtBu)—OH | (6.00 eg) | HATU (5.70 eq) and DIEA (12.00 eq) |
| 17 | Fmoc-Asp(OtBu)—OH | (6.00 eq) | HATU (5.70 eq) and DIEA (12.00 eq) |
| 18 | Fmoc-Gly-OH | (6.00 eg) | HATU (5.70 eq) and DIEA (12.00 eq) |
| 19 | Fmoc-Lys(Boc)-OH | (6.00 eq) | HATU (5.70 eq) and DIEA (12.00 eq) |
| 20 | Fmoc-Lys(Boc)-OH | (6.00 eg) | HATU (5.70 eq) and DIEA (12.00 eq) |
| 21 | Fmoc-Met-OH | (6.00 eg) | HATU (5.70 eq) and DIEA (12.00 eg) |
| 22 | Fmoc-Phe-OH | (6.00 eg) | HATU (5.70 eq) and DIEA (12.00 eq) |
| 23 | Fmoc-Glu(tBu)—OH | (6.00 eq) | HATU (5.70 eq) and DIEA (12.00 eg) |
| 24 | Fmoc-Tyr(tBu)—OH | (6.00 eg) | HATU (5.70 eq) and DIEA (12.00 eq) |
| 25 | Fmoc-Ile-OH | (6.00 eg) | HATU (5.70 eq) and DIEA (12.00 eg) |
| 26 | Fmoc-Leu-OH | (6.00 eg) | HATU (5.70 eq) and DIEA (12.00 eq) |
| 27 | Fmoc-Asp(OtBu)—OH | (6.00 eg) | HATU (5.70 eq) and DIEA (12.00 eq) |
| 28 | Fmoc-Ser(tBu)—OH | (6.00 eg) | HATU (5.70 eg) and DIEA (12.00 eq) |
| 29 | Fmoc-Val-OH | (6.00 eq) | HATU (5.70 eq) and DIEA (12.00 eq) |
| 30 | Fmoc-Arg(Pbf)-OH | (6.00 eq) | HATU (5.70 eq) and DIEA (12.00 eg) |
| 31 | Fmoc-Met-OH | (6.00 eg) | HATU (5.70 eq) and DIEA (12.00 eq) |
| 32 | Fmoc-Ser(tBu)—OH | (6.00 eq) | HATU (5.70 eq) and DIEA (12.00 eg) |
| 33 | Fmoc-Ala-OH | (6.00 eq) | HATU (5.70 eq) and DIEA (12.00 eq) |
| 34 | Fmoc-Glu(OtBu)—OH | (6.00 eq) | HATU (5.70 eq) and DIEA (12.00 eq) |
| 35 | Fmoc-Ala-OH | (6.00 eq) | HATU (5.70 eq) and DIEA (12.00 eg) |
| 36 | Fmoc-His(Trt)-OH | (6.00 eg) | HATU (5.70 eq) and DIEA (12.00 eg) |
| 37 | Fmoc-Gly-OH | (6.00 eq) | HATU (5.70 eq) and DIEA (12.00 eg) |
| 38 | Fmoc-Leu-OH | (6.00 eg) | HATU (5.70 eq) and DIEA (12.00 eg) |
| 39 | Fmoc-Glu(OtBu)—OH | (6.00 eq) | HATU (5.70 eq) and DIEA (12.00 eq) |
| 40 | Fmoc-Asp(OtBu)—OH | (6.00 eg) | HATU (5.70 eq) and DIEA (12.00 eg) |
| 41 | Fmoc-Leu-OH | (6.00 eg) | HATU (5.70 eq) and DIEA (12.00 eq) |
| 42 | Fmoc-Leu-OH | (6.00 eg) | HATU (5.70 eq) and DIEA (12.00 eq) |
| 43 | Fmoc-Arg(Pbf)-OH | (6.00 eq) | HATU (5.70 eg) and DIEA (12.00 eq) |
| 44 | Fmoc-Met-OH | (6.00 eg) | HATU (5.70 eq) and DIEA (12.00 eq) |
| 45 | Fmoc-Ile-OH | (6.00 eg) | HATU (5.70 eg) and DIEA (12.00 eg) |
| 46 | Fmoc-Glu(OtBu)—OH | (6.00 eg) | HATU (5.70 eq) and DIEA (12.00 eg) |
| 47 | Fmoc-Tyr(tBu)—OH | (6.00 eq) | HATU (5.70 eq) and DIEA (12.00 eq) |
| 48 | Fmoc-Ile-OH | (6.00 eg) | HATU (5.70 eq) and DIEA (12.00 eq) |
| 49 | Fmoc-Lys(Boc)-OH | (6.00 eg) | HATU (5.70 eq) and DIEA (12.00 eq) |
| 50 | Fmoc-Gln(Trt)-OH | (6.00 eq) | HATU (5.70 eq) and DIEA (12.00 eq) |
| 51 | Fmoc-Leu-OH | (6.00 eq) | HATU (5.70 eq) and DIEA (12.00 eq) |
| 52 | Fmoc-Ile-OH | (6.00 eg) | HATU (5.70 eq) and DIEA (12.00 eq) |
| 53 | Fmoc-Trp-OH | (6.00 eg) | HATU (5.70 eq) and DIEA (12.00 eq) |
| 54 | Fmoc-Glu(OtBu)—OH | (6.00 eg) | HATU (5.70 eq) and DIEA (12.00 eq) |
| 55 | Fmoc-Lys(Boc)-OH | (6.00 eg) | HATU (5.70 eq) and DIEA (12.00 eg) |
| 56 | Fmoc-Asp(OtBu)—OH | (6.00 eq) | HATU (5.70 eq) and DIEA (12.00 eq) |

### Peptide Cleavage and Purification:

1) Cleavage buffer (95% TFA/2.5 TIS/2.5H₂O, 20.0 mL) was added to the flask containing the side chain protected peptide resin at room temperature and stirred for 2 hrs.
2) The filtrate was collected.
3) The peptide was precipitated with cold isopropyl ether and centrifuged (3 mins at 3000 rpm).
4) Solid was washed with isopropyl ether for another two times.
5) The crude peptide was dried under vacuum 2 hrs.
6) The crude peptide was purified by Prep_HPLC (A: 0.075% TFA in H₂O, B: ACN), followed by lyophilization to give compound I-4 (20.5 mg, 90.10 purity, 3.0 yield, TFA salt) as a white solid.

### Purification Conditions:

Separation condition

| Dissolution | Dissolve in ACN/H₂O | |
|---|---|---|
| condition | | |
| Instrument | GX-281 | |
| Mobile | A: | H₂O (0.075% TFA) |
| Phase | B: | CH₃CN |
| Gradient | 30-60%-51 min. Retention time: 33 min | |
| Column | Luna 100*25 mm, C18, 10 um, 100 Å + | |
| | Gemini ® 150*30 mm, C18, 5 um, 110 Å | |
| Flow Rate | 20 | mL/min |
| Wavelength | 220/254 | nm |

| | |
|---|---|
| Oven Temp. | Room temperature |

Compound I-5 is the N-terminus acetylation version of compound 1-4, while compound 1-6 is Biotin modification on N-terminus NH—₂ group. Similar SPPS methodology was utilized. N-terminus acetylation was achieved by reacting with Ac₂O/NMM/DCM (10/5/85; v/v/v) for 15 min. Biotin modification was achieved by Fmoc-PEG4-COOH coupling on NH—₂ first (Fmoc-PEG4-COOH/HBTU/DIEA, 2.0 eq/1.85 eq/4.0 eq, 30 min), then remove the Fmoc by 20% piperidine, following by Biotin-OSu (2.0 eq) reacting with this exposed NH₂. For each of I-4, I-5 and I-6, expected mass was observed in MS.

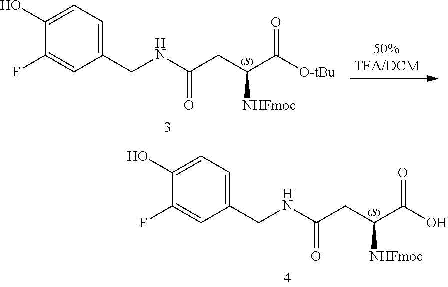

### Example 11. Certain Technologies for Preparing Agents: I-7 and I-8

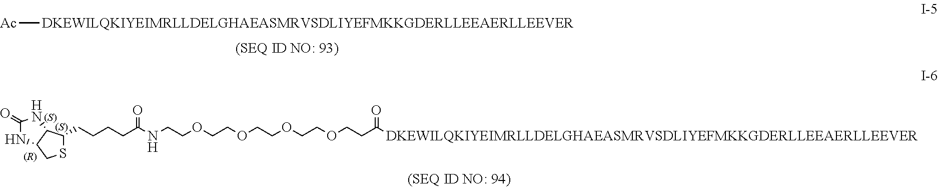

A mixture of compound 1 (10 g, 64.45 mmol) in HBr/H₂O (40% HBr, 300 mL in total) was stirred at 140° C. for 16 hrs. The solvent was removed at 70° C. under reduced pressure, the residue was triturated in MeCN (50 mL) for 10 mins. After filtered, the solid was dried under lyophilization to get compound 2 (13.0 g, 58.5 mmol, 90.8% yield, HBr salt) as a brown solid. ¹H NMR: (400 MHz DMSO-d₆) δ ppm 10.04 (s, 1H) 8.18 (s, 3H) 7.32 (dd, J=12.17, 1.88 Hz, 1H) 7.11 (dd, J=8.28, 1.51 Hz, 1H) 6.96-7.03 (m, 1H) 3.93 (q, J=5.52 Hz, 2H).

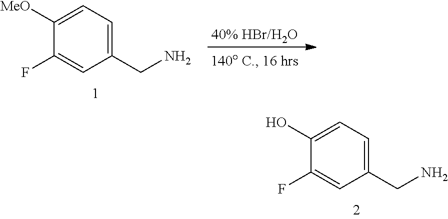 [SMILES: COc1ccc(CN)cc1F ; NCc1ccc(O)c(F)c1]

To a mixture of compound 2 (13.0 g, 58.5 mmol, 1 eq, HBr), compound 2a (24.1 g, 58.5 mmol, 1 eq), DIEA (3.78 g, 29.2 mmol, 5.10 mL, 0.5 eq) and HOBt (11.87 g, 87.8 mmol, 1.5 eq) in DMF (200 mL) was added EDCI (12.35 g, 64.4 mmol, 1.1 eq) at 15° C., the mixture was stirred at 15° C. for 3 hr. The mixture was dropwise added to 0.5 M HCl (cold, 1 L) and white solid was precipitated. After filtered, the solid was dried under lyophilization to afford compound 3 (31 g, crude) as a white solid.

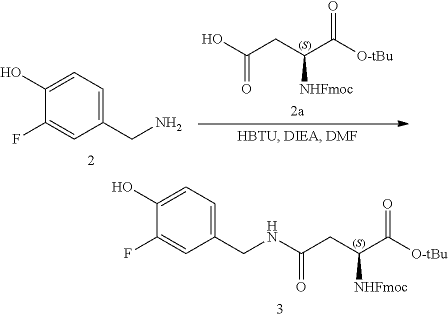

A mixture of compound 3 (30 g, 56.12 mmol, 1.0 eq) in TFA (300 mL) and DCM (300 mL) was stirred at 15° C. for 0.5 hr. The solvent was removed under reduced pressure. The residue was purified by flash C18 (ISCO®; 120 g SepaFlash® C18 Flash Column, Eluent of 0˜90% MeCN/H₂O gradient @ 75 mL/min) directly to get compound 4 (18 g, 37.6 mmol, 67.0% yield) as a white solid.

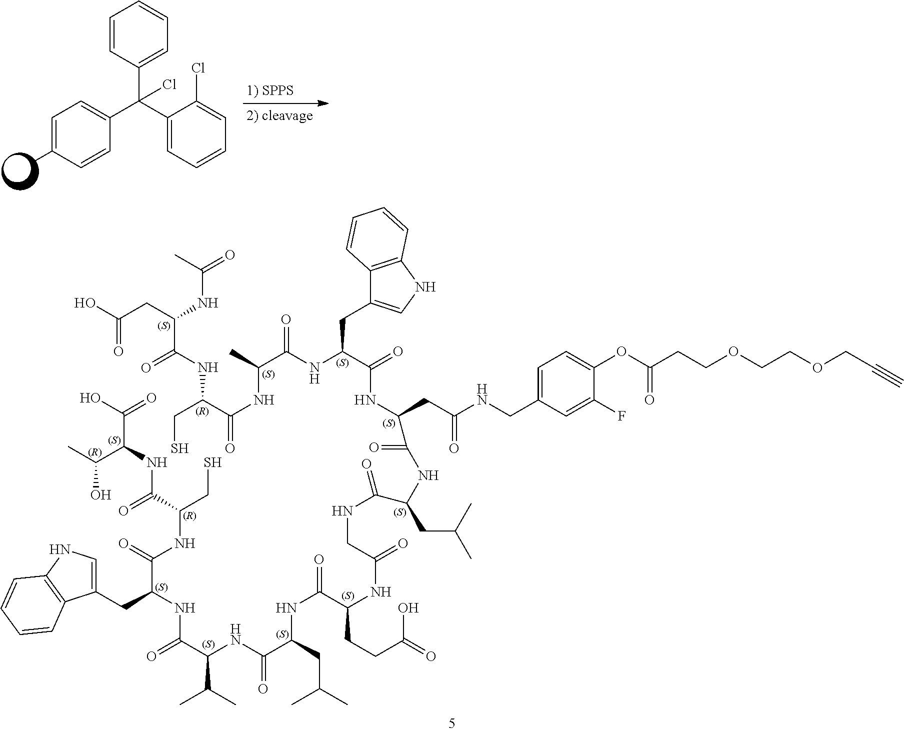

### Peptide was Synthesized Using Standard Fmoc Chemistry.

1) Resin preparation: To the vessel containing CTC Resin (3.0 mmol, 3.0 g, 1.00 mmol/g) and Fmoc-Thr(tBu)-OH (1.19 g, 3.0 mmol, 1.00 eq) in DCM (30 mL) was added DIEA (4.00 eq) dropwise and mixed for 2 hrs with N₂ bubbling at 15° C. Then MeOH (3.0 mL) was added and bubbled with N₂ for another 30 mins. The resin was washed with DMF (60 mL)*5, followed by adding 20% piperidine in DMF (60 mL) and bubbled with N₂ for 30 mins at 15° C. for Fmoc deprotection. The mixture was filtered and the resin was washed with DMF (60 mL)*5 before proceeding to next step.
2) Coupling: A solution of Fmoc-Cys(Trt)-OH (5.25 g, 3.00 eq), HBTU (3.24 g, 2.85 eq) in DMF (30 mL) was added to the resin with N₂ bubbling. Then DIEA (6.00 eq) was added to the mixture dropwise and bubbled with N₂ for 30 mins at 15° C. The coupling reaction was monitored by ninhydrin test, if it showed colorless, the coupling was completed. The resin was then washed with DMF (60 mL)*5.
3) Deprotection: 20% piperidine in DMF (60 mL) was added to the resin and the mixture was bubbled with N₂ for 30 mins at 15° C. The deprotection reaction was monitored by ninhydrin test, if it showed blue or other brownish red, the reaction was completed. The resin was then washed with DMF (60 mL)*5.
4) Step 2 and 3 were repeated for amino acids: (2-8 in table below).
5) Coupling for compound 4: A solution of compound 4 (2.87 g, 2.00 eq), DIC (0.76 g, 2.00 eq) and HOBt (0.82 g, 2.00 eq) in DMF (30 mL) was added to the resin with N₂ bubbling for 60 mins at 15° C. The coupling reaction was monitored by ninhydrin test, if it showed colorless, the coupling was completed. The resin was then washed with DMF (60 mL)*5.
6) Repeat step 3 for Fmoc deprotection.
7) Step 5 and 6 were repeated for amino acids (10-13 in table below).
8) Acetylation: A solution of 10% Ac₂O/5% NMM/85% DMF (60 mL) was added to resin and the mixture was bubbled with N₂ for 20 mins. The acetylation reaction was monitored by ninhydrin test, if it showed colorless, the coupling was completed. The resin was then washed with DMF (60 mL)*5.
9) 3% hydrazine in DMF (60 mL) was added to resin with N₂ bubbling for 15 min to liberate hydroxyl group on compound 4, and resin was then washed with DMF (60 mL)*5.
10) Coupling on hydroxyl group of compound 4: A solution of 3-(2-(prop-2-yn-1-yloxy)ethoxy)propanoic acid (1.54 g, 9.0 mmol, 3.00 eq), DIC (1.13 g, 3.00 eq), HOBt (1.23 g, 3.00 eq) and DMAP (1.10 g, 3.00 eq) was added to resin and the mixture was bubbled with N₂ for 36 hrs. The coupling reaction was monitored by LCMS after a mini-cleavage, where 50% was desired MS. The resin was then washed with DMF (60 mL)*5, MeOH (60 mL)*5, and then dried under vacuum.

| # | Materials | | Coupling reagents |
|---|---|---|---|
| 1 | Fmoc-Thr(tBu)—OH | (1.00 eg) | DIEA (4.00 eg) |
| 2 | Fmoc-Cys(Trt)-OH | (3.00 eg) | HBTU (2.85 eq) and DIEA (6.00 eq) |
| 3 | Fmoc-Trp-OH | (3.00 eq) | HBTU (2.85 eq) and DIEA (6.00 eg) |
| 4 | Fmoc-Val-OH | (3.00 eg) | HBTU (2.85 eq) and DIEA (6.00 eq) |
| 5 | Fmoc-Leu-OH | (3.00 eq) | HBTU (2.85 eq) and DIEA (6.00 eq) |
| 6 | Fmoc-Glu(OtBu)—OH | (3.00 eg) | HBTU (2.85 eq) and DIEA (6.00 eg) |
| 7 | Fmoc-Gly-OH | (3.00 eq) | HBTU (2.85 eq) and DIEA (6.00 eg) |
| 8 | Fmoc-Leu-OH | (3.00 eg) | HBTU (2.85 eq) and DIEA (6.00 eq) |
| 9 | compound 4 | (2.00 eq) | DIC (2.00 eq) and HOBt (2.00 eq) |
| 10 | Fmoc-Trp-OH | (3.00 eq) | DIC (3.00 eq) and HOBt (3.00 eq) |
| 11 | Fmoc-Ala-OH | (3.00 eq) | DIC (3.00 eq) and HOBt (3.00 eq) |
| 12 | Fmoc-Cys(Trt)-OH | (3.00 eq) | DIC (3.00 eq) and HOBt (3.00 eq) |
| 13 | Fmoc-Asp(OtBu)—OH | (3.00 eg) | DIC (3.00 eq) and HOBt (3.00 eq) |
| 14 | Acetylation | | Ac₂O/NMM/DMF (10/5/85, 60 mL) |
| 15 | 3-(2-(prop-2-yn-1-yloxy) | (3.00 eq) | DIC (3.00 eq), HOBt (3.00 eq) and |
| | ethoxy)propanoic acid | | DMAP (3.00 eq) |

### Peptide Cleavage and Purification:

1) Cleavage buffer (95% TFA/2.5% TIS/2.5% H₂O, 60.0 mL) was added to the flask containing the side chain protected peptide resin at room temperature and stirred for 1 hr.
2) The filtrate was collected.
3) The peptide was precipitated with cold isopropyl ether (300 mL) and centrifuged (3 mins at 3000 rpm).
4) Isopropyl ether washed two additional times, and the crude peptide was dried under vacuum for 2 hrs.
5) Compound 5 (4.2 g, crude) was obtained as a white solid.

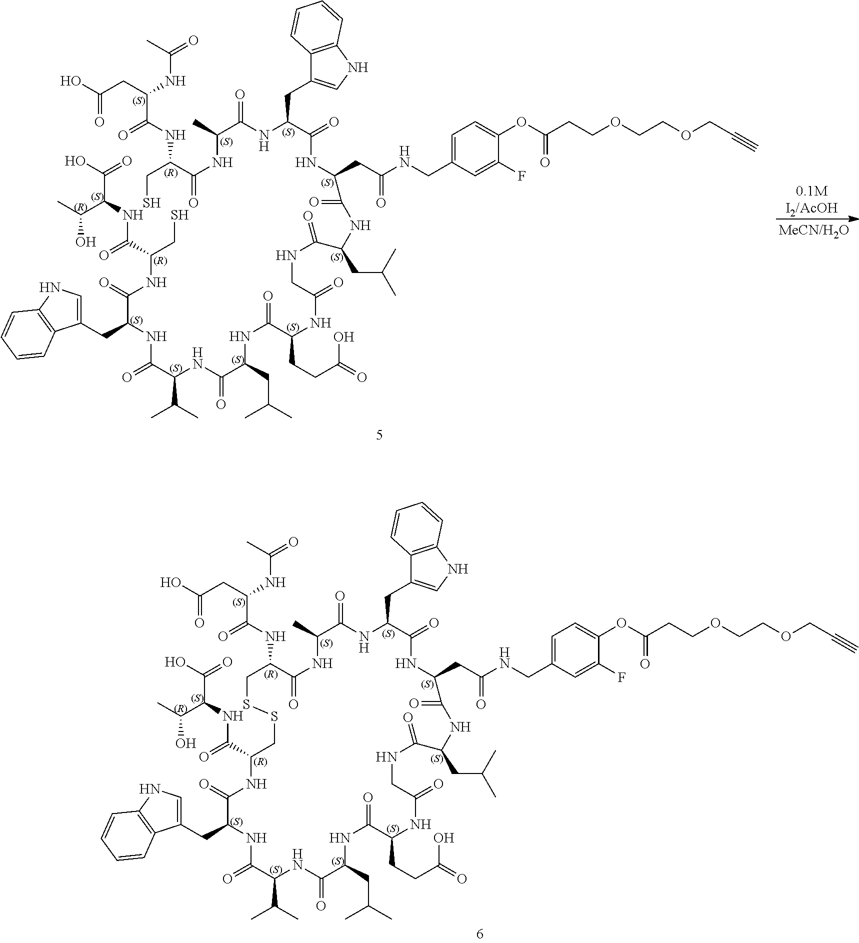

To a mixture of compound 5 (4.2 g, crude) in MeCN/H₂O (1/1, 3.0 L) was added 0.1 M I₂/HOAc dropwise until the yellow color persisted, then the mixture was stirred at 15° C. for 5 mins. The mixture was quenched with 0.1 M Na₂S₂O₃ dropwise until yellow color disappeared. The mixture was lyophilized to give the crude powder. The crude peptide was purified by prep-HPLC (acid condition, TFA), followed by lyophilization to afford compound 6 (500 mg, 90.1% purity, 9.11% yield) as a white solid.

In some embodiments, compound 6 may be utilized as a compound of formula R-I. In some embodiments, it may be utilized to introduce a reactive group comprising —N₃ to another agent, e.g., an antibody agent. In some embodiments, it is useful to prepare an agent comprising an antibody binding moiety, a target binding moiety, and a reactive group.

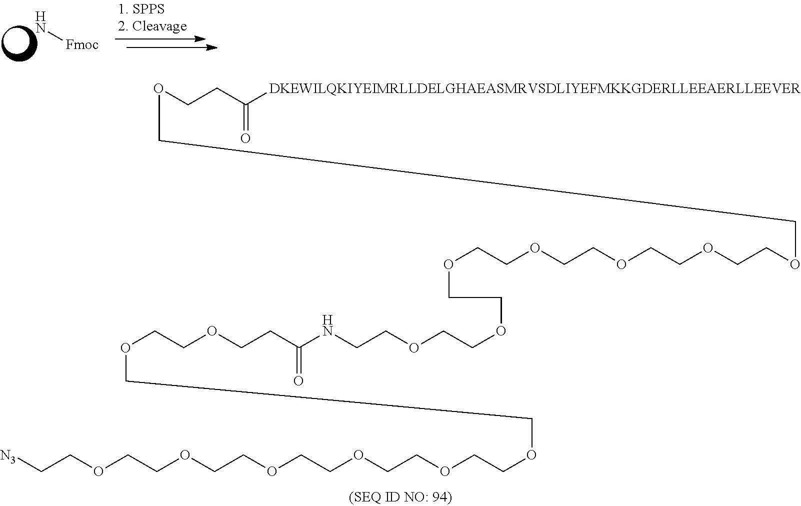

### Peptide was Synthesized Using Standard Fmoc Chemistry.

1) Resin preparation: To the vessel containing CTC Resin (0.10 mmol, 0.13 g, 0.78 mmol/g) and Fmoc-Arg(Pbf)-OH (64.9 mg, 0.10 mmol, 1.00 eq) in DCM (2.0 mL) was added DIEA (4.00 eq) dropwise and mixed for 2 hrs with N₂ bubbling at 15° C. Then MeOH (0.10 mL) was added and bubbled with N₂ for another 30 mins. The resin was washed with DMF (10.0 mL)*5, followed by adding 20% piperidine in DMF (10.0 mL) and bubbled with N₂ for 30 mins at 15° C. for Fmoc deprotection. The mixture was filtered and the resin was washed with DMF (10.0 mL)*5 before proceeding to next step.
2) Coupling: A solution of Fmoc-Glu(OtBu)-OH (0.13 g, 3.00 eq), HBTU (0.11 g, 2.85 eq) in DMF (2.0 mL) was added to the resin with N₂ bubbling. Then DIEA (6.00 eq) was added to the mixture dropwise and bubbled with N₂ for 30 mins at 15° C. The coupling reaction was monitored by ninhydrin test, if the resin showed blue or brownish red, double coupling should be performed; if it showed colorless, the coupling was completed. The resin was then washed with DMF (10.0 mL)*5.
3) Deprotection: 20% piperidine in DMF (10.0 mL) was added to the resin and the mixture was bubbled with N₂ for 30 mins at 15° C. The deprotection reaction was monitored by ninhydrin test, if it showed blue or other brownish red, the reaction was completed. The resin was then washed with DMF (10.0 mL)*5.
4) Step 2 to 3 was repeated for all other amino acids: (2-58 in table below), double coupling was performed for amino acids (Arg8, Glu12, Leu14, Asp17, Phe22, Arg43, Ile45, Glu53, Glu54 in table below).
5) After the last N₃-PEG₈-CH₂CH₂COOH coupled, the resin was then washed with DMF (10.0 mL)*5 MeOH (10.0 mL)*5, and then dried under vacuum.

| # | Materials | | Coupling reagents |
|---|---|---|---|
| 1 | Fmoc-Arg(Pbf)-OH | (1.00 eq) | DIEA (4.00 eg) |
| 2 | Fmoc-Glu(OtBu)—OH | (3.00 eq) | HBTU (2.85 eq) and DIEA (6.00 eg) |
| 3 | Fmoc-Val-OH | (3.00 eq) | HBTU (2.85 eq) and DIEA (6.00 eq) |
| 4 | Fmoc-Glu(OtBu)—OH | (3.00 eg) | HBTU (2.85 eq) and DIEA (6.00 eg) |
| 5 | Fmoc-Glu(OtBu)—OH | (3.00 eg) | HBTU (2.85 eq) and DIEA (6.00 eq) |
| 6 | Fmoc-Leu-OH | (3.00 eq) | HBTU (2.85 eq) and DIEA (6.00 eq) |
| 7 | Fmoc-Leu-OH | (3.00 eg) | HBTU (2.85 eq) and DIEA (6.00 eg) |
| 8 | Fmoc-Arg(Pbf)-OH | (3.00 eg) | HBTU (2.85 eq) and DIEA (6.00 eg) |
| 9 | Fmoc-Glu(OtBu)—OH | (6.00 eg) | HATU (5.70 eq) and DIEA (12.00 eq) |
| 10 | Fmoc-Ala-OH | (6.00 eg) | HATU (5.70 eq) and DIEA (12.00 eq) |
| 11 | Fmoc-Glu(OtBu)—OH | (6.00 eg) | HATU (5.70 eg) and DIEA (12.00 eq) |
| 12 | Fmoc-Glu(OtBu)—OH | (6.00 eq) | HATU (5.70 eq) and DIEA (12.00 eq) |
| 13 | Fmoc-Leu-OH | (6.00 eg) | HATU (5.70 eq) and DIEA (12.00 eq) |
| 14 | Fmoc-Leu-OH | (6.00 eg) | HATU (5.70 eg) and DIEA (12.00 eq) |
| 15 | Fmoc-Arg(Pbf)-OH | (6.00 eq) | HATU (5.70 eq) and DIEA (12.00 eq) |
| 16 | Fmoc-Glu(OtBu)—OH | (6.00 eg) | HATU (5.70 eq) and DIEA (12.00 eg) |
| 17 | Fmoc-Asp(OtBu)—OH | (6.00 eq) | HATU (5.70 eg) and DIEA (12.00 eg) |
| 18 | Fmoc-Gly-OH | (6.00 eg) | HATU (5.70 eg) and DIEA (12.00 eg) |
| 19 | Fmoc-Lys(Boc)-OH | (6.00 eg) | HATU (5.70 eq) and DIEA (12.00 eg) |
| 20 | Fmoc-Lys(Boc)-OH | (6.00 eg) | HATU (5.70 eq) and DIEA (12.00 eg) |
| 21 | Fmoc-Met-OH | (6.00 eq) | HATU (5.70 eq) and DIEA (12.00 eg) |
| 22 | Fmoc-Phe-OH | (6.00 eg) | HATU (5.70 eq) and DIEA (12.00 eg) |
| 23 | Fmoc-Glu(tBu)—OH | (6.00 eg) | HATU (5.70 eq) and DIEA (12.00 eg) |
| 24 | Fmoc-Tyr(tBu)—OH | (6.00 eg) | HATU (5.70 eq) and DIEA (12.00 eq) |
| 25 | Fmoc-Ile-OH | (6.00 eg) | HATU (5.70 eq) and DIEA (12.00 eg) |
| 26 | Fmoc-Leu-OH | (6.00 eg) | HATU (5.70 eq) and DIEA (12.00 eq) |
| 27 | Fmoc-Asp(OtBu)—OH | (6.00 eg) | HATU (5.70 eq) and DIEA (12.00 eg) |
| 28 | Fmoc-Ser(tBu)—OH | (6.00 eg) | HATU (5.70 eq) and DIEA (12.00 eq) |
| 29 | Fmoc-Val-OH | (6.00 eg) | HATU (5.70 eg) and DIEA (12.00 eg) |
| 30 | Fmoc-Arg(Pbf)-OH | (6.00 eg) | HATU (5.70 eq) and DIEA (12.00 eg) |
| 31 | Fmoc-Met-OH | (6.00 eq) | HATU (5.70 eq) and DIEA (12.00 eg) |
| 32 | Fmoc-Ser(Bu)—OH | (6.00 eg) | HATU (5.70 eq) and DIEA (12.00 eq) |
| 33 | Fmoc-Ala-OH | (6.00 eg) | HATU (5.70 eq) and DIEA (12.00 eg) |
| 34 | Fmoc-Glu(OtBu)—OH | (6.00 eg) | HATU (5.70 eq) and DIEA (12.00 eq) |
| 35 | Fmoc-Ala-OH | (6.00 eg) | HATU (5.70 eq) and DIEA (12.00 eg) |
| 36 | Fmoc-His(Trt)-OH | (6.00 eq) | HATU (5.70 eg) and DIEA (12.00 eg) |
| 37 | Fmoc-Gly-OH | (6.00 eq) | HATU (5.70 eq) and DIEA (12.00 eg) |
| 38 | Fmoc-Leu-OH | (6.00 eq) | HATU (5.70 eq) and DIEA (12.00 eg) |
| 39 | Fmoc-Glu(OtBu)—OH | (6.00 eg) | HATU (5.70 eq) and DIEA (12.00 eg) |
| 40 | Fmoc-Asp(OtBu)—OH | (6.00 eq) | HATU (5.70 eq) and DIEA (12.00 eq) |
| 41 | Fmoc-Leu-OH | (6.00 eg) | HATU (5.70 eq) and DIEA (12.00 eg) |
| 42 | Fmoc-Leu-OH | (6.00 eg) | HATU (5.70 eq) and DIEA (12.00 eq) |
| 43 | Fmoc-Arg(Pbf)-OH | (6.00 eq) | HATU (5.70 eq) and DIEA (12.00 eg) |
| 44 | Fmoc-Met-OH | (6.00 eq) | HATU (5.70 eg) and DIEA (12.00 eq) |
| 45 | Fmoc-Ile-OH | (6.00 eg) | HATU (5.70 eq) and DIEA (12.00 eg) |
| 46 | Fmoc-Glu(OtBu)—OH | (6.00 eg) | HATU (5.70 eq) and DIEA (12.00 eg) |
| 47 | Fmoc-Tyr(Bu)—OH | (6.00 eg) | HATU (5.70 eq) and DIEA (12.00 eq) |
| 48 | Fmoc-Ile-OH | (6.00 eg) | HATU (5.70 eq) and DIEA (12.00 eg) |
| 49 | Fmoc-Lys(Boc)-OH | (6.00 eq) | HATU (5.70 eq) and DIEA (12.00 eg) |
| 50 | Fmoc-Gln(Trt)-OH | (6.00 eg) | HATU (5.70 eq) and DIEA (12.00 eg) |
| 51 | Fmoc-Leu-OH | (6.00 eg) | HATU (5.70 eq) and DIEA (12.00 eq) |
| 52 | Fmoc-Ile-OH | (6.00 eg) | HATU (5.70 eq) and DIEA (12.00 eg) |
| 53 | Fmoc-Trp-OH | (6.00 eg) | HATU (5.70 eq) and DIEA (12.00 eq) |
| 54 | Fmoc-Glu(OtBu)—OH | (6.00 eq) | HATU (5.70 eq) and DIEA (12.00 eq) |
| 55 | Fmoc-Lys(Boc)-OH | (6.00 eg) | HATU (5.70 eq) and DIEA (12.00 eq) |
| 56 | Fmoc-Asp(OtBu)—OH | (6.00 eg) | HATU (5.70 eq) and DIEA (12.00 eq) |
| 57 | FmocNH-PEG₈-CH₂CH₂COOH | (3.00 eg) | HATU (2.85 eq) and DIEA (6.00 eg) |
| 58 | N₃-PEG₈-CH₂CH₂COOH | (3.00 eq) | HATU (2.85 eq) and DIEA (6.00 eq) |

### Peptide Cleavage and Purification:

1) Cleavage buffer (95% TFA/2.5% TIS/2.5% H₂O, 20.0 mL) was added to the flask containing the side chain protected peptide resin at room temperature and stirred for 2 hrs.
2) The filtrate was collected.
3) The peptide was precipitated with cold isopropyl ether and centrifuged (3 mins at 3000 rpm).
4) Isopropyl ether washed two additional times.
5) The crude peptide was dried under vacuum 2 hrs.
6) The crude peptide was purified by Prep_HPLC (A: 0.075% TFA in H₂O, B: ACN), followed by lyophilization to give compound 7 (57.0 mg, 95.0% purity, 7.42% yield) as a white solid.

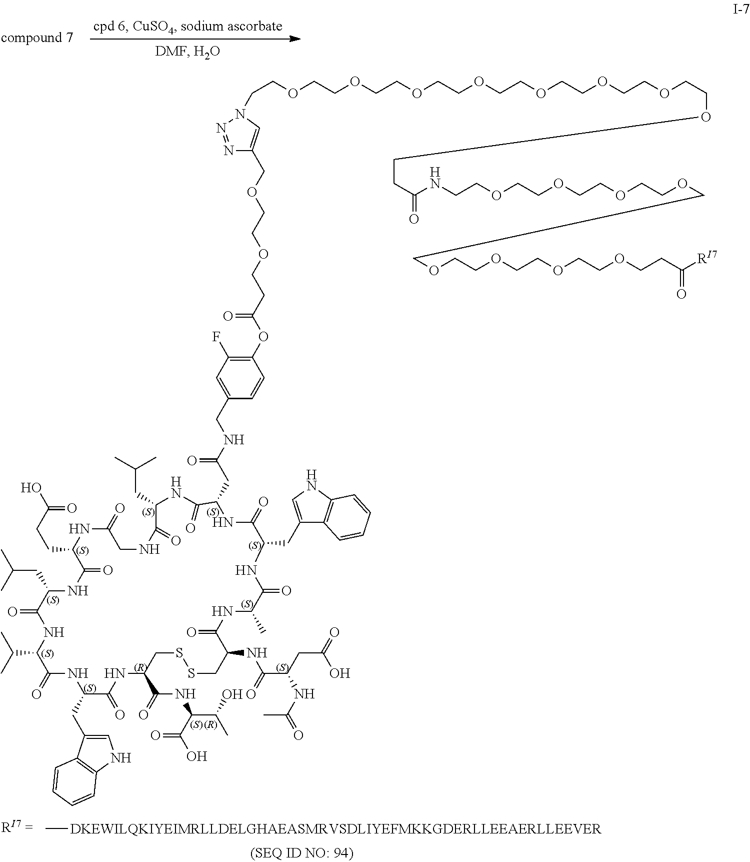

To a mixture of compound 7 (57.0 mg, 7.05 umol, 95.0% purity, 1.00 eq) and compound 6 (15.4 mg, 8.46 umol, 1.20 eq) in DMF (0.50 mL) was added a mixture of CuSO₄ (0.40 M, 17.6 uL, 1.00 eq), sodium ascorbate (5.6 mg, 28.1 umol, 4.00 eq), and THPTA (6.1 mg, 14.09 umol, 2.00 eq) in NH₄HCO₃ (0.20 M, 105.7 uL, 3.00 eq) at 15° C. under N₂ atmosphere. Then the mixture was stirred at 15° C. for 1 hr under N₂ atmosphere. The mixture was purified by prep-HPLC (acid condition, TFA) directly to afford compound I-7 (8.6 mg, 0.78 umol, 87.1% purity, 11.17% yield for click step, TFA salt) as a white solid.

### Purification Conditions:

Separation condition

| Dissolution | Dissolve in ACN/H₂O | |
|---|---|---|
| condition | | |
| Instrument | GX-281 | |
| Mobile | A: | H₂O (0.075% TFA) |
| Phase | B: | CH₃CN |
| Gradient | 30-60%-51 min. Retention time: 30 min | |
| Column | Luna 100*25 mm, C18, 10 um, 100 Å + | |
| | Gemini ® 150*30 mm, C18, 5 um, 110 Å | |
| Flow Rate | 20 | mL/min |
| Wavelength | 220/254 | nm |

| | |
|---|---|
| Oven Temp. | Room temperature |

Compound I-8 was synthesized via the same methodology, wherein a corresponding PEG4 material was used in cycle 57 and 58 in the table above. For each of I-7 and I-8, expected MS was observed.

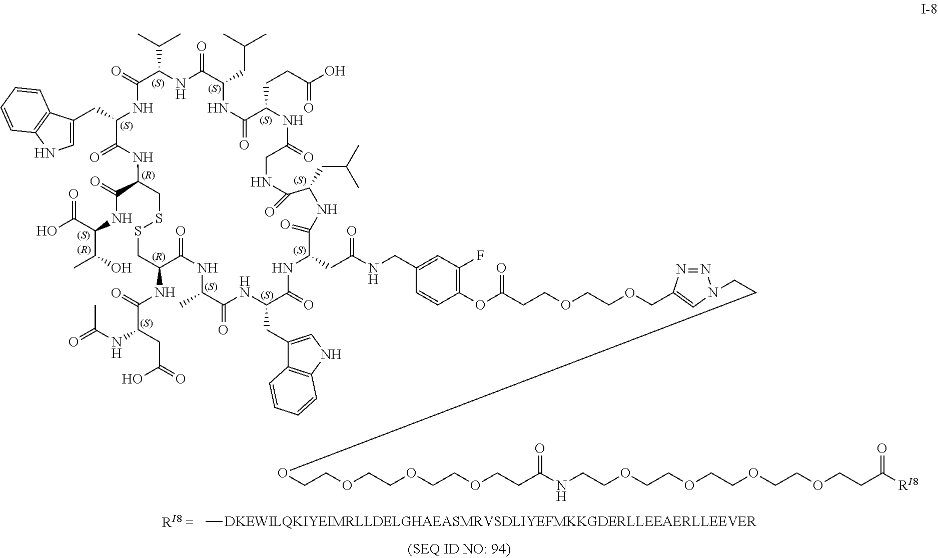

### Example 12. Certain Technologies for Preparing Agents: I-9, I-10, and I-11

I-9
(SEQ ID NO: 6)
NDDELHMLMTDLVYEALHFAKDEEIKKRVFQLFELADKAYKNNDRQKLE
KVVEELKELLERLLS

### Peptide was Synthesized Using Standard Fmoc Chemistry.

1) Resin preparation: To the vessel containing CTC Resin (0.10 mmol, 0.13 g, 0.78 mmol/g) and Fmoc-Ser(tBu)-OH (38.3 mg, 0.10 mmol, 1.00 eq) in DCM (2.0 mL) was added DIEA (4.00 eq) dropwise and mixed for 2 hrs with N₂ bubbling at 15° C. Then MeOH (0.10 mL) was added and bubbled with N₂ for another 30 mins. The resin was washed with DMF (10.0 mL)*5, followed by adding 20% piperidine in DMF (10.0 mL) and bubbled with N₂ for 30 mins at 15° C. for Fmoc deprotection. The mixture was filtered and the resin was washed with DMF (10.0 mL)*5 before proceeding to next step.
2) Coupling: A solution of Fmoc-Leu-OH (0.11 g, 3.00 eq), HBTU (0.11 g, 2.85 eq) in DMF (2.0 mL) was added to the resin with N₂ bubbling. Then DIEA (6.00 eq) was added to the mixture dropwise and bubbled with N₂ for 30 mins at 15° C. The coupling reaction was monitored by ninhydrin test, if the resin showed blue or brownish red, double coupling should be performed; if it showed colorless, the coupling was completed. The resin was then washed with DMF (10.0 mL)*5.
3) Deprotection: 20% piperidine in DMF (10.0 mL) was added to the resin and the mixture was bubbled with N₂ for 30 mins at 15° C. The deprotection reaction was monitored by ninhydrin test, if it showed blue or other brownish red, the reaction was completed. The resin was then washed with DMF (10.0 mL)*5.
4) Step 2 and 3 were repeated for amino acids: (2-64 in table below), double coupling was performed for amino acids (Glu8, Arg20, Ala29, Phe35, Arg37, Lys44, His47, Tyr51, Tyr55, His59, Asp62, Asp63 and Asn64 in table below).
5) After de-Fmoc of the last amino acid, the resin was then washed with DMF (10.0 mL)*5, MeOH (10.0 mL)*5, and then dried under vacuum.

| # | Materials | | Coupling reagents |
|---|---|---|---|
| 1 | Fmoc-Ser(tBu)—OH | (1.00 eq) | DIEA (4.00 eq) |
| 2 | Fmoc-Leu-OH | (3.00 eq) | HBTU (2.85 eq) and DIEA (6.00 eq) |
| 3 | Fmoc-Leu-OH | (3.00 eg) | HBTU (2.85 eq) and DIEA (6.00 eq) |
| 4 | Fmoc-Arg(Pbf)-OH | (3.00 eg) | HBTU (2.85 eq) and DIEA (6.00 eq) |
| 5 | Fmoc-Glu(OtBu)—OH | (3.00 eq) | HBTU (2.85 eq) and DIEA (6.00 eq) |
| 6 | Fmoc-Leu-OH | (3.00 eg) | HBTU (2.85 eq) and DIEA (6.00 eq) |
| 7 | Fmoc-Leu-OH | (3.00 eg) | HBTU (2.85 eq) and DIEA (6.00 eq) |
| 8 | Fmoc-Glu(OtBu)—OH | (3.00 eg) | HBTU (2.85 eq) and DIEA (6.00 eg) |
| 9 | Fmoc-Lys(Boc)-OH | (6.00 eg) | HATU (5.70 eq) and DIEA (12.00 eg) |
| 10 | Fmoc-Leu-OH | (6.00 eq) | HATU (5.70 eq) and DIEA (12.00 eq) |
| 11 | Fmoc-Glu(OtBu)—OH | (6.00 eg) | HATU (5.70 eq) and DIEA (12.00 eq) |
| 12 | Fmoc-Glu(OtBu)—OH | (6.00 eg) | HATU (5.70 eq) and DIEA (12.00 eq) |
| 13 | Fmoc-Val-OH | (6.00 eg) | HATU (5.70 eq) and DIEA (12.00 eg) |
| 14 | Fmoc-Val-OH | (6.00 eg) | HATU (5.70 eq) and DIEA (12.00 eq) |
| 15 | Fmoc-Lys(Boc)-OH | (6.00 eq) | HATU (5.70 eq) and DIEA (12.00 eq) |
| 16 | Fmoc-Glu(OtBu)—OH | (6.00 eg) | HATU (5.70 eq) and DIEA (12.00 eg) |
| 17 | Fmoc-Leu-OH | (6.00 eq) | HATU (5.70 eq) and DIEA (12.00 eq) |
| 18 | Fmoc-Lys(Boc)-OH | (6.00 eg) | HATU (5.70 eq) and DIEA (12.00 eg) |
| 19 | Fmoc-Gln(Trt)-OH | (6.00 eq) | HATU (5.70 eq) and DIEA (12.00 eq) |
| 20 | Fmoc-Arg(Pbf)-OH | (6.00 eq) | HATU (5.70 eq) and DIEA (12.00 eg) |
| 21 | Fmoc-Asp(OtBu)—OH | (6.00 eg) | HATU (5.70 eq) and DIEA (12.00 eq) |
| 22 | Fmoc-Asn(Trt)-OH | (6.00 eg) | HATU (5.70 eq) and DIEA (12.00 eg) |
| 23 | Fmoc-Asn(Trt)-OH | (6.00 eg) | HATU (5.70 eq) and DIEA (12.00 eq) |
| 24 | Fmoc-Lys(Boc)-OH | (6.00 eq) | HATU (5.70 eq) and DIEA (12.00 eg) |
| 25 | Fmoc-Tyr(tBu)—OH | (6.00 eg) | HATU (5.70 eq) and DIEA (12.00 eq) |
| 26 | Fmoc-Ala-OH | (6.00 eg) | HATU (5.70 eq) and DIEA (12.00 eg) |
| 27 | Fmoc-Lys(Boc)-OH | (6.00 eg) | HATU (5.70 eq) and DIEA (12.00 eq) |
| 28 | Fmoc-Asp(OtBu)—OH | (6.00 eg) | HATU (5.70 eq) and DIEA (12.00 eg) |
| 29 | Fmoc-Ala-OH | (6.00 eq) | HATU (5.70 eq) and DIEA (12.00 eg) |
| 30 | Fmoc-Leu-OH | (6.00 eq) | HATU (5.70 eq) and DIEA (12.00 eq) |
| 31 | Fmoc-Glu(OtBu)—OH | (6.00 eg) | HATU (5.70 eq) and DIEA (12.00 eg) |
| 32 | Fmoc-Phe-OH | (6.00 eg) | HATU (5.70 eq) and DIEA (12.00 eg) |
| 33 | Fmoc-Leu-OH | (6.00 eq) | HATU (5.70 eq) and DIEA (12.00 eq) |
| 34 | Fmoc-Gln(Trt)-OH | (6.00 eq) | HATU (5.70 eg) and DIEA (12.00 eq) |
| 35 | Fmoc-Phe-OH | (6.00 eg) | HATU (5.70 eq) and DIEA (12.00 eq) |
| 36 | Fmoc-Val-OH | (6.00 eq) | HATU (5.70 eg) and DIEA (12.00 eg) |
| 37 | Fmoc-Arg(Pbf)-OH | (6.00 eg) | HATU (5.70 eq) and DIEA (12.00 eq) |
| 38 | Fmoc-Lys(Boc)-OH | (6.00 eg) | HATU (5.70 eq) and DIEA (12.00 eg) |
| 39 | Fmoc-Lys(Boc)-OH | (6.00 eg) | HATU (5.70 eq) and DIEA (12.00 eq) |
| 40 | Fmoc-Ile-OH | (6.00 eg) | HATU (5.70 eg) and DIEA (12.00 eg) |
| 41 | Fmoc-Glu(OtBu)—OH | (6.00 eg) | HATU (5.70 eq) and DIEA (12.00 eq) |
| 42 | Fmoc-Glu(OtBu)—OH | (6.00 eg) | HATU (5.70 eq) and DIEA (12.00 eg) |
| 43 | Fmoc-Asp(OtBu)—OH | (6.00 eg) | HATU (5.70 eq) and DIEA (12.00 eq) |
| 44 | Fmoc-Lys(Boc)-OH | (6.00 eg) | HATU (5.70 eq) and DIEA (12.00 eq) |
| 45 | Fmoc-Ala-OH | (6.00 eq) | HATU (5.70 eq) and DIEA (12.00 eq) |
| 46 | Fmoc-Phe-OH | (6.00 eg) | HATU (5.70 eq) and DIEA (12.00 eq) |
| 47 | Fmoc-His(Trt)-OH | (6.00 eg) | HATU (5.70 eq) and DIEA (12.00 eq) |
| 48 | Fmoc-Leu-OH | (6.00 eq) | HATU (5.70 eq) and DIEA (12.00 eq) |
| 49 | Fmoc-Ala-OH | (6.00 eg) | HATU (5.70 eq) and DIEA (12.00 eq) |
| 50 | Fmoc-Glu(OtBu)—OH | (6.00 eq) | HATU (5.70 eq) and DIEA (12.00 eq) |
| 51 | Fmoc-Tyr(tBu)—OH | (6.00 eq) | HATU (5.70 eq) and DIEA (12.00 eq) |
| 52 | Fmoc-Val-OH | (6.00 eq) | HATU (5.70 eq) and DIEA (12.00 eq) |
| 53 | Fmoc-Leu-OH | (6.00 eg) | HATU (5.70 eq) and DIEA (12.00 eg) |
| 54 | Fmoc-Asp(OtBu)—OH | (6.00 eq) | HATU (5.70 eq) and DIEA (12.00 eq) |
| 55 | Fmoc-Thr(tBu)—OH | (6.00 eg) | HATU (5.70 eq) and DIEA (12.00 eg) |
| 56 | Fmoc-Met-OH | (6.00 eq) | HATU (5.70 eq) and DIEA (12.00 eq) |
| 57 | Fmoc-Leu-OH | (6.00 eq) | HATU (5.70 eq) and DIEA (12.00 eq) |
| 58 | Fmoc-Met-OH | (6.00 eg) | HATU (5.70 eg) and DIEA (12.00 eq) |
| 59 | Fmoc-His(Trt)-OH | (6.00 eg) | HATU (5.70 eq) and DIEA (12.00 eg) |
| 60 | Fmoc-Leu-OH | (6.00 eq) | HATU (5.70 eq) and DIEA (12.00 eg) |
| 61 | Fmoc-Glu(OtBu)—OH | (6.00 eg) | HATU (5.70 eq) and DIEA (12.00 eg) |
| 62 | Fmoc-Asp(OtBu)—OH | (6.00 eg) | HATU (5.70 eq) and DIEA (12.00 eq) |
| 63 | Fmoc-Asp(OtBu)—OH | (6.00 eq) | HATU (5.70 eq) and DIEA (12.00 eg) |
| 64 | Fmoc-Asn(Trt)-OH | (6.00 eq) | HATU (5.70 eq) and DIEA (12.00 eq) |

### Peptide Cleavage and Purification:

1) Cleavage buffer (95% TFA/2.5% TIS/2.5% H₂O, 20.0 mL) was added to the flask containing the side chain protected peptide resin at room temperature and stirred for 2 hrs.
2) The filtrate was collected.
3) The peptide was precipitated with cold isopropyl ether and centrifuged (3 mins at 3000 rpm).
4) The solid was washed with isopropyl ether for another two times.
5) The crude peptide was dried under vacuum 2 hrs.
6) The crude peptide was purified by Prep_HPLC (A: 0.075% TFA in H₂O, B: ACN), followed by lyophilization to give compound I-9 (47.8 mg, 87.5% purity, 6.18% yield, TFA salt) as a white solid.

### Purification Conditions:

Separation condition

| Dissolution | Dissolve in ACN/H₂O | |
|---|---|---|
| condition | | |
| Instrument | GX-281 | |
| Mobile | A: | H₂O (0.075% TFA) |
| Phase | B: | CH₃CN |
| Gradient | 30-60%-51 min. Retention time: 35 min | |
| Column | Luna 100*25 mm, C18, 10 um, 100 Å + | |
| | Gemini ® 150*30 mm, C18, 5 um, 110 Å | |
| Flow Rate | 20 | mL/min |
| Wavelength | 220/254 | nm |

| | |
|---|---|
| Oven Temp. | Room temperature |

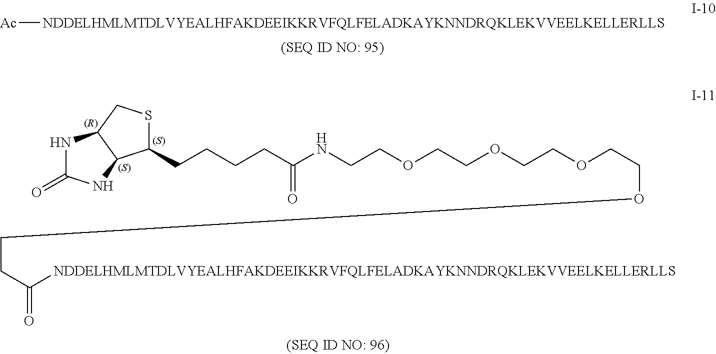

Compound I-10 was the N-terminus acetylation version of compound I-9, while compound I-11 has biotin modification on N-terminus NH₂ group. SPPS methodology is the same. N-terminus acetylation was achieved by reacting with Ac₂O/NMM/DCM (10/5/85; v/v/v) for 15 min. Biotin modification was achieved by Fmoc-PEG4-COOH coupling on NH₂ first (Fmoc-PEG4-COOH/HBTU/DIEA, 2.0 eq/1.85 eq/4.0 eq, 30 min), then removing the Fmoc by 20% piperidine, followed by Biotin-OSu (2.0 eq) reacting with this exposed NH₂. In one preparation of I-10, a purity of 90% was observed. In one preparation of I-11, a purity of 89% was observed. For each of I-9, I-10, and I-11, expected mass was observed.

### Example 13. Certain Technologies for Preparing Agents: I-12, I-13, and I-14

I-12
(SEQ ID NO: 97)
DKEWILQKIYEIMRLLDELGHAEASMRVSDLIYEFMKKGDERLLEEAER
LLEEVER-NH2

### Peptide was Synthesized Using Standard Fmoc Chemistry.

1) Resin preparation: DMF (10.0 mL) was added to the vessel containing Rink amide AM Resin (sub: 0.263 mmol/g, 0.10 mmol, 0.38 g) and swelled for 2 hrs.
2) Deprotection: 20% piperidine/DMF was added to the resin with N₂ bubbling for about 0.5 hr. The deprotection reaction was monitored by ninhydrin test, if it showed blue or other brownish red, the reaction was completed. The resin was then washed with DMF (20.0 mL)*5.
3) Coupling: Fmoc-amino acid solution in DMF (10.0 mL) was added to resin with N₂ bubbling, then activation buffer was added to the resin with N₂ bubbling for about 1 hr. The coupling reaction was monitored by ninhydrin test, if it showed blue or brownish red, double coupling would be performed; if it showed colorless, the coupling was completed. The resin was then washed with DMF (20.0 mL)*5.
4) Step 2 and 3 were repeated for amino acids: (2-56 in table below), double coupling was performed for amino acids (Arg8, Asp17, Ile25, Ser28, Arg30, His36, Asp40, Arg43, Glu46, Leu51, Lys55, and Asp56 in table below).
5) After de-Fmoc of the last amino acid, the resin was then washed with DMF (20.0 mL)*5, MeOH (20.0 mL)*5, and then dried under vacuum.

| # | Materials | | Coupling reagents |
|---|---|---|---|
| 1 | Fmoc-Arg(Pbf)-OH | (3.00 eg) | HBTU (2.85 eq) and DIEA (6.00 eq) |
| 2 | Fmoc-Glu(OtBu)—OH | (3.00 eg) | HBTU (2.85 eq) and DIEA (6.00 eq) |
| 3 | Fmoc-Val-OH | (3.00 eq) | HBTU (2.85 eq) and DIEA (6.00 eq) |
| 4 | Fmoc-Glu(OtBu)—OH | (3.00 eq) | HBTU (2.85 eq) and DIEA (6.00 eq) |
| 5 | Fmoc-Glu(OtBu)—OH | (3.00 eq) | HBTU (2.85 eq) and DIEA (6.00 eq) |
| 6 | Fmoc-Leu-OH | (3.00 eg) | HBTU (2.85 eg) and DIEA (6.00 eq) |
| 7 | Fmoc-Leu-OH | (3.00 eq) | HBTU (2.85 eq) and DIEA (6.00 eq) |
| 8 | Fmoc-Arg(Pbf)-OH | (3.00 eg) | HBTU (2.85 eg) and DIEA (6.00 eg) |
| 9 | Fmoc-Glu(OtBu)—OH | (6.00 eg) | HATU (5.70 eq) and DIEA (12.00 eq) |
| 10 | Fmoc-Ala-OH | (6.00 eg) | HATU (5.70 eq) and DIEA (12.00 eg) |
| 11 | Fmoc-Glu(OtBu)—OH | (6.00 eq) | HATU (5.70 eq) and DIEA (12.00 eq) |
| 12 | Fmoc-Glu(OtBu)—OH | (6.00 eg) | HATU (5.70 eq) and DIEA (12.00 eq) |
| 13 | Fmoc-Leu-OH | (6.00 eq) | HATU (5.70 eq) and DIEA (12.00 eq) |
| 14 | Fmoc-Leu-OH | (6.00 eq) | HATU (5.70 eq) and DIEA (12.00 eg) |
| 15 | Fmoc-Arg(Pbf)-OH | (6.00 eg) | HATU (5.70 eq) and DIEA (12.00 eq) |
| 16 | Fmoc-Glu(OtBu)—OH | (6.00 eg) | HATU (5.70 eq) and DIEA (12.00 eq) |
| 17 | Fmoc-Asp(OtBu)—OH | (6.00 eg) | HATU (5.70 eq) and DIEA (12.00 eq) |
| 18 | Fmoc-Gly-OH | (6.00 eg) | HATU (5.70 eq) and DIEA (12.00 eq) |
| 19 | Fmoc-Lys(Boc)-OH | (6.00 eg) | HATU (5.70 eq) and DIEA (12.00 eq) |
| 20 | Fmoc-Lys(Boc)-OH | (6.00 eg) | HATU (5.70 eq) and DIEA (12.00 eq) |
| 21 | Fmoc-Met-OH | (6.00 eg) | HATU (5.70 eq) and DIEA (12.00 eg) |
| 22 | Fmoc-Phe-OH | (6.00 eq) | HATU (5.70 eq) and DIEA (12.00 eq) |
| 23 | Fmoc-Glu(tBu)—OH | (6.00 eg) | HATU (5.70 eq) and DIEA (12.00 eg) |
| 24 | Fmoc-Tyr(tBu)—OH | (6.00 eg) | HATU (5.70 eq) and DIEA (12.00 eq) |
| 25 | Fmoc-Ile-OH | (6.00 eg) | HATU (5.70 eq) and DIEA (12.00 eg) |
| 26 | Fmoc-Leu-OH | (6.00 eq) | HATU (5.70 eq) and DIEA (12.00 eq) |
| 27 | Fmoc-Asp(OtBu)—OH | (6.00 eg) | HATU (5.70 eq) and DIEA (12.00 eq) |
| 28 | Fmoc-Ser(tBu)—OH | (6.00 eg) | HATU (5.70 eq) and DIEA (12.00 eq) |
| 29 | Fmoc-Val-OH | (6.00 eg) | HATU (5.70 eq) and DIEA (12.00 eg) |
| 30 | Fmoc-Arg(Pbf)-OH | (6.00 eq) | HATU (5.70 eq) and DIEA (12.00 eg) |
| 31 | Fmoc-Met-OH | (6.00 eg) | HATU (5.70 eq) and DIEA (12.00 eq) |
| 32 | Fmoc-Ser(Bu)—OH | (6.00 eg) | HATU (5.70 eq) and DIEA (12.00 eg) |
| 33 | Fmoc-Ala-OH | (6.00 eq) | HATU (5.70 eq) and DIEA (12.00 eq) |
| 34 | Fmoc-Glu(OtBu)—OH | (6.00 eq) | HATU (5.70 eq) and DIEA (12.00 eq) |
| 35 | Fmoc-Ala-OH | (6.00 eq) | HATU (5.70 eq) and DIEA (12.00 eg) |
| 36 | Fmoc-His(Trt)-OH | (6.00 eg) | HATU (5.70 eq) and DIEA (12.00 eg) |
| 37 | Fmoc-Gly-OH | (6.00 eq) | HATU (5.70 eq) and DIEA (12.00 eq) |
| 38 | Fmoc-Leu-OH | (6.00 eg) | HATU (5.70 eq) and DIEA (12.00 eg) |
| 39 | Fmoc-Glu(OtBu)—OH | (6.00 eg) | HATU (5.70 eq) and DIEA (12.00 eq) |
| 40 | Fmoc-Asp(OtBu)—OH | (6.00 eg) | HATU (5.70 eq) and DIEA (12.00 eq) |
| 41 | Fmoc-Leu-OH | (6.00 eq) | HATU (5.70 eq) and DIEA (12.00 eq) |
| 42 | Fmoc-Leu-OH | (6.00 eg) | HATU (5.70 eq) and DIEA (12.00 eg) |
| 43 | Fmoc-Arg(Pbf)-OH | (6.00 eg) | HATU (5.70 eq) and DIEA (12.00 eq) |
| 44 | Fmoc-Met-OH | (6.00 eg) | HATU (5.70 eq) and DIEA (12.00 eq) |
| 45 | Fmoc-Ile-OH | (6.00 eg) | HATU (5.70 eq) and DIEA (12.00 eg) |
| 46 | Fmoc-Glu(OtBu)—OH | (6.00 eg) | HATU (5.70 eq) and DIEA (12.00 eg) |
| 47 | Fmoc-Tyr(tBu)—OH | (6.00 eg) | HATU (5.70 eq) and DIEA (12.00 eq) |
| 48 | Fmoc-Ile-OH | (6.00 eg) | HATU (5.70 eq) and DIEA (12.00 eq) |
| 49 | Fmoc-Lys(Boc)-OH | (6.00 eg) | HATU (5.70 eq) and DIEA (12.00 eg) |
| 50 | Fmoc-Gln(Trt)-OH | (6.00 eg) | HATU (5.70 eg) and DIEA (12.00 eg) |
| 51 | Fmoc-Leu-OH | (6.00 eq) | HATU (5.70 eq) and DIEA (12.00 eg) |
| 52 | Fmoc-Ile-OH | (6.00 eq) | HATU (5.70 eq) and DIEA (12.00 eq) |
| 53 | Fmoc-Trp-OH | (6.00 eg) | HATU (5.70 eq) and DIEA (12.00 eg) |
| 54 | Fmoc-Glu(OtBu)—OH | (6.00 eq) | HATU (5.70 eq) and DIEA (12.00 eq) |
| 55 | Fmoc-Lys(Boc)-OH | (6.00 eq) | HATU (5.70 eg) and DIEA (12.00 eq) |
| 56 | Fmoc-Asp(OtBu—OH | (6.00 eg) | HATU (5.70 eg) and DIEA (12.00 eg) |

### Peptide Cleavage and Purification:

1) Cleavage buffer (95% TFA/2.5% TIS/2.5% H₂O, 30.0 mL) was added to the flask containing the side chain protected peptide resin at room temperature and stirred for 2 hrs.
2) The filtrate was collected.
3) The peptide was precipitated with cold isopropyl ether and centrifuged (3 mins at 3000 rpm).
4) The solid was washed with isopropyl ether for another two times.
5) The crude peptide was dried under vacuum 2 hrs.
6) The crude peptide was purified by Prep_HPLC (A: 0.075% TFA in H₂O, B: ACN), followed by lyophilization to give compound I-12 (44.5 mg, 91.8% purity, 6.53% yield, TFA salt) as a white solid.

### Purification Conditions:

Separation condition

| Dissolution | Dissolve in ACN/H₂O | |
|---|---|---|
| condition | | |
| Instrument | GX-281 | |
| Mobile | A: | H₂O (0.075% TFA) |
| Phase | B: | CH₃CN |
| Gradient | 30-60%-51 min. Retention time: 33 min | |
| Column | Luna 100*25 mm, C18, 10 um, 100 Å + | |
| | Gemini ® 150*30 mm, C18, 5 um, 110 Å | |
| Flow Rate | 20 | mL/min |
| Wavelength | 220/254 | nm |

| | |
|---|---|
| Oven Temp. | Room temperature |

Compound I-13 is the N-terminus acetylation version of compound I-12, while compound I-14 is Biotin modification on N-terminus NH₂ group. SPPS methodology was the same. N-terminus acetylation was achieved by reacting with Ac₂O/NMM/DCM (10/5/85; v/v/v) for 15 min. Biotin modification was achieved by Fmoc-PEG4-COOH coupling on NH₂ first (Fmoc-PEG4-COOH/HBTU/DIEA, 2.0 eq/1.85 eq/4.0 eq, 30 min), then removing the Fmoc by 20% piperidine, followed by Biotin-OSu (2.0 eq) reacting with this exposed NH₂. In one preparation of I-13, 95% purity was observed. In one preparation of I-14, 95% purity was observed. For each of I-12, I-13 and I-14, expected mass was observed.

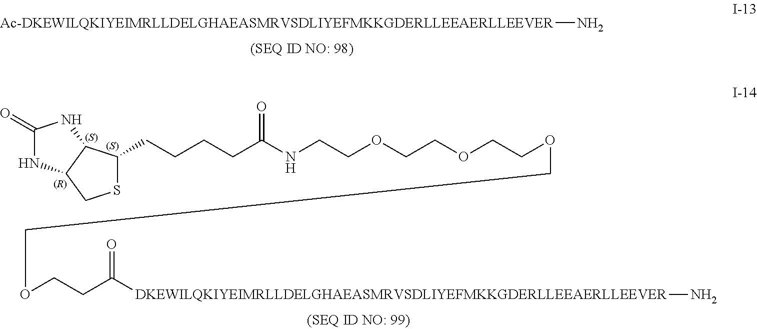

### Example 14. Certain Technologies for Preparing Agents: I-15

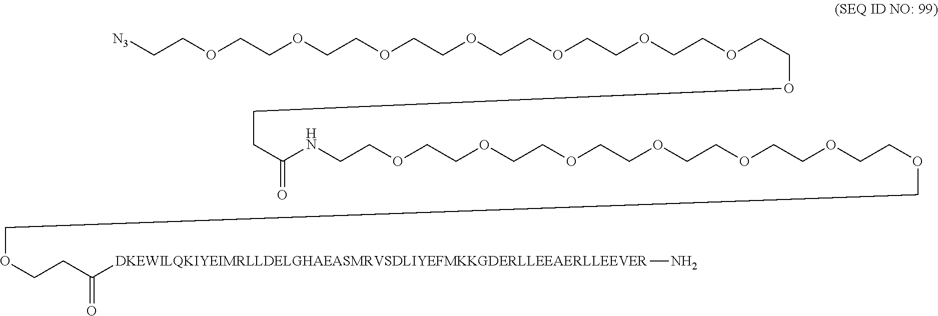

### Compound 7 was Synthesized Using Standard Fmoc Chemistry.

1) Resin preparation: DMF (10.0 mL) was added to the vessel containing Rink amide AM Resin (sub: 0.263 mmol/g, 0.10 mmol, 0.38 g) and swelled for 2 hrs.
2) Deprotection: 20% piperidine/DMF was added to the resin with N₂ bubbling for about 0.5 hr. The de-protection reaction was monitored by ninhydrin test, if it showed blue or other brownish red, the reaction was completed. The resin was then washed with DMF (20.0 mL)*5.
3) Coupling: Fmoc-amino acid solution in DMF (10.0 mL) was added to resin with N₂ bubbling, then activation buffer was added to the resin with N₂ bubbling for about 1 hr. The coupling reaction was monitored by ninhydrin test, if it showed blue or brownish red, double coupling would be performed; if it showed colorless, the coupling was completed. The resin was then washed with DMF (20.0 mL)*5.
4) Step 2 and 3 were repeated for amino acids: (2-56 in table below), double coupling was performed for amino acids (Arg8, Asp17, Ile25, Ser28, Arg30, His36, Asp40, Arg43, Glu46, Leu51, Lys55, and Asp56 in table below).
5) After the last N₃-PEG₈-CH₂CH₂COOH coupled, the resin was then washed with DMF (20.0 mL)*5, MeOH (20.0 mL)*5, and then dried under vacuum.

| # | Materials | | Coupling reagents |
|---|---|---|---|
| 1 | Fmoc-Arg(Pbf)-OH | (3.00 eg) | HBTU (2.85 eq) and DIEA (6.00 eg) |
| 2 | Fmoc-Glu(OtBu)—OH | (3.00 eg) | HBTU (2.85 eg) and DIEA (6.00 eg) |
| 3 | Fmoc-Val-OH | (3.00 eq) | HBTU (2.85 eq) and DIEA (6.00 eg) |
| 4 | Fmoc-Glu(OtBu)—OH | (3.00 eg) | HBTU (2.85 eq) and DIEA (6.00 eg) |
| 5 | Fmoc-Glu(OtBu)—OH | (3.00 eg) | HBTU (2.85 eq) and DIEA (6.00 eg) |
| 6 | Fmoc-Leu-OH | (3.00 eg) | HBTU (2.85 eq) and DIEA (6.00 eg) |
| 7 | Fmoc-Leu-OH | (3.00 eq) | HBTU (2.85 eq) and DIEA (6.00 eg) |
| 8 | Fmoc-Arg(Pbf)-OH | (3.00 eg) | HBTU (2.85 eq) and DIEA (6.00 eg) |
| 9 | Fmoc-Glu(OtBu)—OH | (6.00 eq) | HATU (5.70 eq) and DIEA (12.00 eq) |
| 10 | Fmoc-Ala-OH | (6.00 eg) | HATU (5.70 eq) and DIEA (12.00 eq) |
| 11 | Fmoc-Glu(OtBu)—OH | (6.00 eq) | HATU (5.70 eq) and DIEA (12.00 eq) |
| 12 | Fmoc-Glu(OtBu)—OH | (6.00 eg) | HATU (5.70 eq) and DIEA (12.00 eq) |
| 13 | Fmoc-Leu-OH | (6.00 eg) | HATU (5.70 eq) and DIEA (12.00 eq) |
| 14 | Fmoc-Leu-OH | (6.00 eg) | HATU (5.70 eg) and DIEA (12.00 eg) |
| 15 | Fmoc-Arg(Pbf)-OH | (6.00 eg) | HATU (5.70 eq) and DIEA (12.00 eq) |
| 16 | Fmoc-Glu(OtBu)—OH | (6.00 eg) | HATU (5.70 eq) and DIEA (12.00 eg) |
| 17 | Fmoc-Asp(OtBu)—OH | (6.00 eg) | HATU (5.70 eq) and DIEA (12.00 eg) |
| 18 | Fmoc-Gly-OH | (6.00 eg) | HATU (5.70 eq) and DIEA (12.00 eg) |
| 19 | Fmoc-Lys(Boc)-OH | (6.00 eg) | HATU (5.70 eq) and DIEA (12.00 eg) |
| 20 | Fmoc-Lys(Boc)-OH | (6.00 eg) | HATU (5.70 eq) and DIEA (12.00 eg) |
| 21 | Fmoc-Met-OH | (6.00 eg) | HATU (5.70 eq) and DIEA (12.00 eg) |
| 22 | Fmoc-Phe-OH | (6.00 eg) | HATU (5.70 eq) and DIEA (12.00 eg) |
| 23 | Fmoc-Glu(Bu)—OH | (6.00 eg) | HATU (5.70 eq) and DIEA (12.00 eg) |
| 24 | Fmoc-Tyr(tBu)—OH | (6.00 eg) | HATU (5.70 eq) and DIEA (12.00 eg) |
| 25 | Fmoc-Ile-OH | (6.00 eq) | HATU (5.70 eq) and DIEA (12.00 eg) |
| 26 | Fmoc-Leu-OH | (6.00 eg) | HATU (5.70 eq) and DIEA (12.00 eq) |
| 27 | Fmoc-Asp(OtBu)—OH | (6.00 eq) | HATU (5.70 eq) and DIEA (12.00 eg) |
| 28 | Fmoc-Ser(tBu)—OH | (6.00 eg) | HATU (5.70 eq) and DIEA (12.00 eq) |
| 29 | Fmoc-Val-OH | (6.00 eg) | HATU (5.70 eq) and DIEA (12.00 eg) |
| 30 | Fmoc-Arg(Pbf)-OH | (6.00 eg) | HATU (5.70 eq) and DIEA (12.00 eq) |
| 31 | Fmoc-Met-OH | (6.00 eg) | HATU (5.70 eq) and DIEA (12.00 eg) |
| 32 | Fmoc-Ser(tBu)—OH | (6.00 eg) | HATU (5.70 eq) and DIEA (12.00 eg) |
| 33 | Fmoc-Ala-OH | (6.00 eq) | HATU (5.70 eq) and DIEA (12.00 eg) |
| 34 | Fmoc-Glu(OtBu)—OH | (6.00 eg) | HATU (5.70 eq) and DIEA (12.00 eq) |
| 35 | Fmoc-Ala-OH | (6.00 eg) | HATU (5.70 eq) and DIEA (12.00 eg) |
| 36 | Fmoc-His(Trt)-OH | (6.00 eg) | HATU (5.70 eq) and DIEA (12.00 eq) |
| 37 | Fmoc-Gly-OH | (6.00 eg) | HATU (5.70 eq) and DIEA (12.00 eg) |
| 38 | Fmoc-Leu-OH | (6.00 eg) | HATU (5.70 eq) and DIEA (12.00 eq) |
| 39 | Fmoc-Glu(OtBu)—OH | (6.00 eg) | HATU (5.70 eq) and DIEA (12.00 eq) |
| 40 | Fmoc-Asp(OtBu)—OH | (6.00 eg) | HATU (5.70 eq) and DIEA (12.00 eq) |
| 41 | Fmoc-Leu-OH | (6.00 eg) | HATU (5.70 eq) and DIEA (12.00 eq) |
| 42 | Fmoc-Leu-OH | (6.00 eg) | HATU (5.70 eq) and DIEA (12.00 eq) |
| 43 | Fmoc-Arg(Pbf)-OH | (6.00 eg) | HATU (5.70 eq) and DIEA (12.00 eq) |
| 44 | Fmoc-Met-OH | (6.00 eq) | HATU (5.70 eq) and DIEA (12.00 eq) |
| 45 | Fmoc-Ile-OH | (6.00 eg) | HATU (5.70 eq) and DIEA (12.00 eq) |
| 46 | Fmoc-Glu(OtBu)—OH | (6.00 eq) | HATU (5.70 eq) and DIEA (12.00 eq) |
| 47 | Fmoc-Tyr(tBu)—OH | (6.00 eg) | HATU (5.70 eq) and DIEA (12.00 eq) |
| 48 | Fmoc-Ile-OH | (6.00 eq) | HATU (5.70 eq) and DIEA (12.00 eg) |
| 49 | Fmoc-Lys(Boc)-OH | (6.00 eq) | HATU (5.70 eg) and DIEA (12.00 eq) |
| 50 | Fmoc-Gln(Trt)-OH | (6.00 eq) | HATU (5.70 eq) and DIEA (12.00 eq) |
| 51 | Fmoc-Leu-OH | (6.00 eg) | HATU (5.70 eq) and DIEA (12.00 eq) |
| 52 | Fmoc-Ile-OH | (6.00 eq) | HATU (5.70 eq) and DIEA (12.00 eq) |
| 53 | Fmoc-Trp-OH | (6.00 eq) | HATU (5.70 eq) and DIEA (12.00 eg) |
| 54 | Fmoc-Glu(OtBu)—OH | (6.00 eg) | HATU (5.70 eq) and DIEA (12.00 eq) |
| 55 | Fmoc-Lys(Boc)-OH | (6.00 eq) | HATU (5.70 eq) and DIEA (12.00 eq) |
| 56 | Fmoc-Asp(OtBu)—OH | (6.00 eq) | HATU (5.70 eq) and DIEA (12.00 eq) |
| 57 | Fmoc-NH-PEG₈-CH₂CH₂COOH | (3.00 eq) | HATU (2.85 eq) and DIEA (6.00 eg) |
| 58 | N₃-PEG8-CH₂CH₂COOH | (3.00 eg) | HATU (2.85 eq) and DIEA (6.00 eg) |

### Peptide Cleavage and Purification:

1) Cleavage buffer (95% TFA/2.5% TIS/2.5% H₂O, 30.0 mL) was added to the flask containing the side chain protected peptide resin at room temperature and stirred for 2 hrs.
2) The filtrate was collected.
3) The peptide was precipitated with cold isopropyl ether and centrifuged (3 mins at 3000 rpm).
4) Isopropyl ether washed two additional times.
5) The crude peptide was dried under vacuum 2 hrs.
6) The crude peptide was purified by Prep_HPLC (A: 0.075% TFA in H₂O, B: ACN), followed by lyophilization to give compound 7 (50.5 mg, 98.8% purity, 6.57% yield, TFA salt) as a white solid.

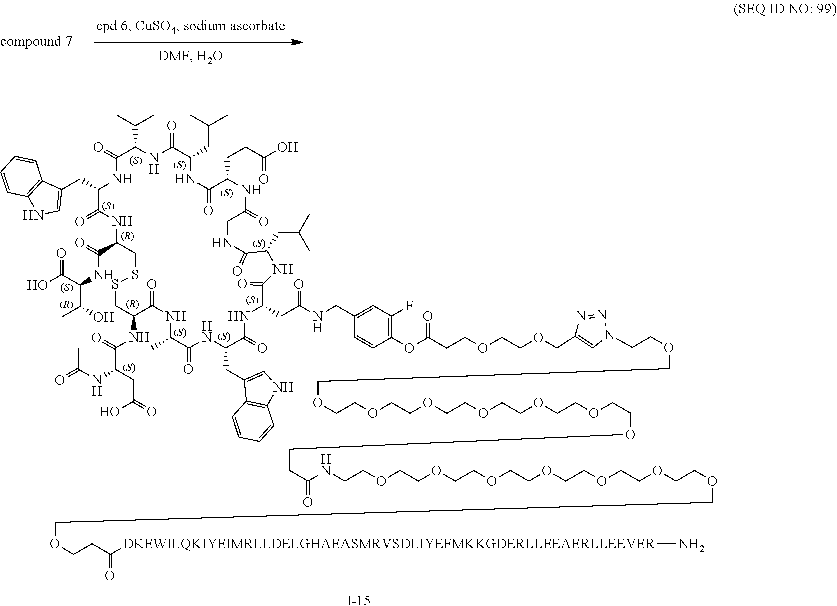

To a mixture of compound 7 (50.0 mg, 6.40 umol, 98.8% purity, 1.00 eq) and compound 6 (19.5 mg, 9.60 umol, 90% purity, 1.50 eq) in DMF (0.50 mL) was added a mixture of CuSO₄ (0.40 M, 16.0 uL, 1.00 eq), sodium ascorbate (5.1 mg, 25.72 umol, 4.00 eq), and THPTA (5.6 mg, 12.8 umol, 2.00 eq) in NH₄HCO₃ (0.20 M, 96.4 uL, 3.00 eq) at 15° C. under N₂ atmosphere. Then the mixture was stirred at 15° C. for 1 hr under N₂ atmosphere. The mixture was purified by prep-HPLC (acid condition, TFA) directly to afford compound I-15 (1.9 mg, 0.17 umol, 86.0% purity, 2.67% yield, TFA salt) as a white solid. Expected mass was observed in MS.

### Purification Conditions:

Separation condition

| Dissolution | Dissolve in ACN/H₂O | |
|---|---|---|
| condition | | |
| Instrument | GX-281 | |
| Mobile | A: | H₂O (TFA 0.075%) |
| Phase | B: | CH₃CN |
| Gradient | 30-60%-51 min. Retention time: 30 min | |
| Column | Luna 100*25 mm, C18, 10 um, 100 Å + | |
| | Gemini ® 150*30 mm, C18, 5 um, 110 Å | |
| Flow Rate | 20 | mL/min |
| Wavelength | 220/254 | nm |

| | |
|---|---|
| Oven Temp. | Room temperature |

### Example 15. Certain Technologies for Preparing Agents: I-16, I-17 and I-18

I-16
(SEQ ID NO: 100)
NDDELHMLMTDLVYEALHFAKDEEIKKRVFQLFELADKAYKNNDRQKLE
KVVEELKELLERLLS-NH2

### I-16 was Synthesized Using Standard Fmoc Chemistry.

1) Resin preparation: DMF (10.0 mL) was added to the vessel containing Rink amide AM Resin (sub: 0.263 mmol/g, 0.1 mmol, 0.38 g) and swelled for 2 hrs.
2) Deprotection: 20% piperidine/DMF was added to the resin with N₂ bubbling for about 0.5 hr. The de-protection reaction was monitored by ninhydrin test, if it showed blue or other brownish red, the reaction was completed. The resin was then washed with DMF (20.0 mL)*5.
3) Coupling: Fmoc-amino acid solution in DMF (10.0 mL) was added to resin with N₂ bubbling, then activation buffer was added to the resin with N₂ bubbling for about 1 hr. The coupling reaction was monitored by ninhydrin test, if it showed blue or brownish red, double coupling would be performed; if it showed colorless, the coupling was completed. The resin was then washed with DMF (20.0 mL)*5.
4) Step 2 and 3 were repeated for amino acids: (2-64 in table below), double coupling was performed for amino acids (Glu8, Arg20, Asp28, Phe35, Arg37, Glu42, His47, Asp54, His59, Asp63, and Asn64 in table below).
5) After de-Fmoc of the last amino acid, the resin was then washed with DMF (20.0 mL)*5, MeOH (20.0 mL)*5, and then dried under vacuum.

| # | Materials | | Coupling reagents |
|---|---|---|---|
| 1 | Fmoc-Ser(tBu)—OH | (3.00 eg) | HBTU (2.85 eq) and DIEA (6.00 eq) |
| 2 | Fmoc-Leu-OH | (3.00 eq) | HBTU (2.85 eq) and DIEA (6.00 eg) |
| 3 | Fmoc-Leu-OH | (3.00 eq) | HBTU (2.85 eq) and DIEA (6.00 eq) |
| 4 | Fmoc-Arg(Pbf)-OH | (3.00 eg) | HBTU (2.85 eq) and DIEA (6.00 eg) |
| 5 | Fmoc-Glu(OtBu)—OH | (3.00 eq) | HBTU (2.85 eq) and DIEA (6.00 eg) |
| 6 | Fmoc-Leu-OH | (3.00 eq) | HBTU (2.85 eq) and DIEA (6.00 eg) |
| 7 | Fmoc-Leu-OH | (3.00 eg) | HBTU (2.85 eq) and DIEA (6.00 eg) |
| 8 | Fmoc-Glu(OtBu)—OH | (3.00 eq) | HBTU (2.85 eq) and DIEA (6.00 eg) |
| 9 | Fmoc-Lys(Boc)-OH | (6.00 eg) | HATU (5.70 eq) and DIEA (12.00 eq) |
| 10 | Fmoc-Leu-OH | (6.00 eq) | HATU (5.70 eg) and DIEA (12.00 eq) |
| 11 | Fmoc-Glu(OtBu)—OH | (6.00 eq) | HATU (5.70 eq) and DIEA (12.00 eq) |
| 12 | Fmoc-Glu(OtBu)—OH | (6.00 eq) | HATU (5.70 eg) and DIEA (12.00 eq) |
| 13 | Fmoc-Val-OH | (6.00 eq) | HATU (5.70 eq) and DIEA (12.00 eq) |
| 14 | Fmoc-Val-OH | (6.00 eg) | HATU (5.70 eq) and DIEA (12.00 eq) |
| 15 | Fmoc-Lys(Boc)-OH | (6.00 eg) | HATU (5.70 eq) and DIEA (12.00 eg) |
| 16 | Fmoc-Glu(OtBu)—OH | (6.00 eq) | HATU (5.70 eq) and DIEA (12.00 eg) |
| 17 | Fmoc-Leu-OH | (6.00 eg) | HATU (5.70 eq) and DIEA (12.00 eq) |
| 18 | Fmoc-Lys(Boc)-OH | (6.00 eg) | HATU (5.70 eq) and DIEA (12.00 eq) |
| 19 | Fmoc-Gln(Trt)-OH | (6.00 eg) | HATU (5.70 eq) and DIEA (12.00 eq) |
| 20 | Fmoc-Arg(Pbf)-OH | (6.00 eg) | HATU (5.70 eq) and DIEA (12.00 eg) |
| 21 | Fmoc-Asp(OtBu)—OH | (6.00 eg) | HATU (5.70 eq) and DIEA (12.00 eg) |
| 22 | Fmoc-Asn(Trt)-OH | (6.00 eg) | HATU (5.70 eq) and DIEA (12.00 eq) |
| 23 | Fmoc-Asn(Trt)-OH | (6.00 eq) | HATU (5.70 eg) and DIEA (12.00 eg) |
| 24 | Fmoc-Lys(Boc)-OH | (6.00 eq) | HATU (5.70 eq) and DIEA (12.00 eq) |
| 25 | Fmoc-Tyr(tBu)—OH | (6.00 eg) | HATU (5.70 eg) and DIEA (12.00 eq) |
| 26 | Fmoc-Ala-OH | (6.00 eq) | HATU (5.70 eq) and DIEA (12.00 eq) |
| 27 | Fmoc-Lys(Boc)-OH | (6.00 eg) | HATU (5.70 eq) and DIEA (12.00 eq) |
| 28 | Fmoc-Asp(OtBu)—OH | (6.00 eg) | HATU (5.70 eg) and DIEA (12.00 eq) |
| 29 | Fmoc-Ala-OH | (6.00 eq) | HATU (5.70 eg) and DIEA (12.00 eg) |
| 30 | Fmoc-Leu-OH | (6.00 eq) | HATU (5.70 eq) and DIEA (12.00 eq) |
| 31 | Fmoc-Glu(OtBu)—OH | (6.00 eg) | HATU (5.70 eg) and DIEA (12.00 eq) |
| 32 | Fmoc-Phe-OH | (6.00 eq) | HATU (5.70 eq) and DIEA (12.00 eg) |
| 33 | Fmoc-Leu-OH | (6.00 eg) | HATU (5.70 eg) and DIEA (12.00 eq) |
| 34 | Fmoc-Gln(Trt)-OH | (6.00 eq) | HATU (5.70 eq) and DIEA (12.00 eg) |
| 35 | Fmoc-Phe-OH | (6.00 eq) | HATU (5.70 eq) and DIEA (12.00 eq) |
| 36 | Fmoc-Val-OH | (6.00 eg) | HATU (5.70 eq) and DIEA (12.00 eq) |
| 37 | Fmoc-Arg(Pbf)-OH | (6.00 eq) | HATU (5.70 eg) and DIEA (12.00 eg) |
| 38 | Fmoc-Lys(Boc)-OH | (6.00 eq) | HATU (5.70 eq) and DIEA (12.00 eq) |
| 39 | Fmoc-Lys(Boc)-OH | (6.00 eg) | HATU (5.70 eq) and DIEA (12.00 eg) |
| 40 | Fmoc-Ile-OH | (6.00 eg) | HATU (5.70 eq) and DIEA (12.00 eq) |
| 41 | Fmoc-Glu(OtBu)—OH | (6.00 eq) | HATU (5.70 eg) and DIEA (12.00 eg) |
| 42 | Fmoc-Glu(OtBu)—OH | (6.00 eq) | HATU (5.70 eq) and DIEA (12.00 eq) |
| 43 | Fmoc-Asp(OtBu)—OH | (6.00 eg) | HATU (5.70 eq) and DIEA (12.00 eq) |
| 44 | Fmoc-Lys(Boc)-OH | (6.00 eg) | HATU (5.70 eq) and DIEA (12.00 eg) |
| 45 | Fmoc-Ala-OH | (6.00 eq) | HATU (5.70 eq) and DIEA (12.00 eq) |
| 46 | Fmoc-Phe-OH | (6.00 eg) | HATU (5.70 eq) and DIEA (12.00 eq) |
| 47 | Fmoc-His(Trt)-OH | (6.00 eq) | HATU (5.70 eq) and DIEA (12.00 eq) |
| 48 | Fmoc-Leu-OH | (6.00 eg) | HATU (5.70 eq) and DIEA (12.00 eg) |
| 49 | Fmoc-Ala-OH | (6.00 eg) | HATU (5.70 eq) and DIEA (12.00 eq) |
| 50 | Fmoc-Glu(OtBu)—OH | (6.00 eq) | HATU (5.70 eq) and DIEA (12.00 eg) |
| 51 | Fmoc-Tyr(tBu)—OH | (6.00 eq) | HATU (5.70 eq) and DIEA (12.00 eq) |
| 52 | Fmoc-Val-OH | (6.00 eq) | HATU (5.70 eq) and DIEA (12.00 eq) |
| 53 | Fmoc-Leu-OH | (6.00 eg) | HATU (5.70 eq) and DIEA (12.00 eg) |
| 54 | Fmoc-Asp(OtBu)—OH | (6.00 eg) | HATU (5.70 eq) and DIEA (12.00 eq) |
| 55 | Fmoc-Thr(tBu)—OH | (6.00 eq) | HATU (5.70 eq) and DIEA (12.00 eq) |
| 56 | Fmoc-Met-OH | (6.00 eg) | HATU (5.70 eq) and DIEA (12.00 eg) |
| 57 | Fmoc-Leu-OH | (6.00 eg) | HATU (5.70 eq) and DIEA (12.00 eg) |
| 58 | Fmoc-Met-OH | (6.00 eq) | HATU (5.70 eq) and DIEA (12.00 eq) |
| 59 | Fmoc-His(Trt)-OH | (6.00 eq) | HATU (5.70 eq) and DIEA (12.00 eq) |
| 60 | Fmoc-Leu-OH | (6.00 eg) | HATU (5.70 eq) and DIEA (12.00 eg) |
| 61 | Fmoc-Glu(OtBu)—OH | (6.00 eq) | HATU (5.70 eq) and DIEA (12.00 eq) |
| 62 | Fmoc-Asp(OtBu)—OH | (6.00 eg) | HATU (5.70 eq) and DIEA (12.00 eg) |
| 63 | Fmoc-Asp(OtBu)—OH | (6.00 eq) | HATU (5.70 eq) and DIEA (12.00 eq) |
| 64 | Fmoc-Asn(Trt)-OH | (6.00 eq) | HATU (5.70 eg) and DIEA (12.00 eq) |

### Peptide Cleavage and Purification:

1) Cleavage buffer (95% TFA/2.5% TIS/2.5% H₂O, 30.0 mL) was added to the flask containing the side chain protected peptide resin at room temperature and stirred for 2 hrs.
2) The filtrate was collected.
3) The peptide was precipitated with cold isopropyl ether and centrifuged (3 mins at 3000 rpm).
4) The solid was washed with isopropyl ether for another two times.
5) The crude peptide was dried under vacuum 2 hrs.
6) The crude peptide was purified by Prep_HPLC (A: 0.075% TFA in H₂O, B: ACN), followed by lyophilization to give compound I-16 (71.5 mg, 88.7% purity, 9.24% yield, TFA salt) as a white solid.

### Purification Conditions:

| | Separation condition |
|---|---|
| Dissolution | Dissolve in ACN/H₂O |
| condition | |
| Instrument | GX-281 |
| Mobile Phase | A: H₂O (0.075% TFA) |
| | B: CH₃CN |
| Gradient | 30-60%-51 min. Retention time: 35 min |
| Column | Luna 100*25 mm, C18, 10 um, 100Å + Gemini ® |
| | 150*30 mm, C18, 5 um, 110Å |
| Flow Rate | 20 mL/min |
| Wavelength | 220/254 nm |
| Oven Temp. | Room temperature |

Compound I-17 is the N-terminus acetylation version of compound I-16, while compound I-18 is biotin modification on N-terminus NH₂ group. SPPS methodology was the same. N-terminus acetylation was achieved by reacting with Ac₂O/NMM/DCM (10/5/85; v/v/v) for 15 min. Biotin modification was achieved by Fmoc-PEG4-COOH coupling on NH₂ first (Fmoc-PEG4-COOH/HBTU/DIEA, 2.0 eq/1.85 eq/4.0 eq, 30 min), then removing the Fmoc by 20% piperidine, followed by Biotin-OSu (2.0 eq) reacting with this exposed NH₂. In one preparation of I-17, 87% purity was observed. In one preparation of I-18, 90% purity was observed. For each of I-16, I-17 and I-18, expected mass was observed.

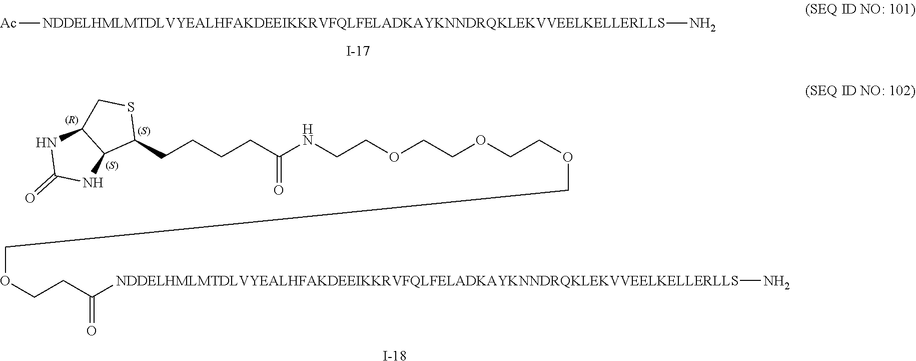

### Example 16. Certain Technologies for Preparing Agents: I-19

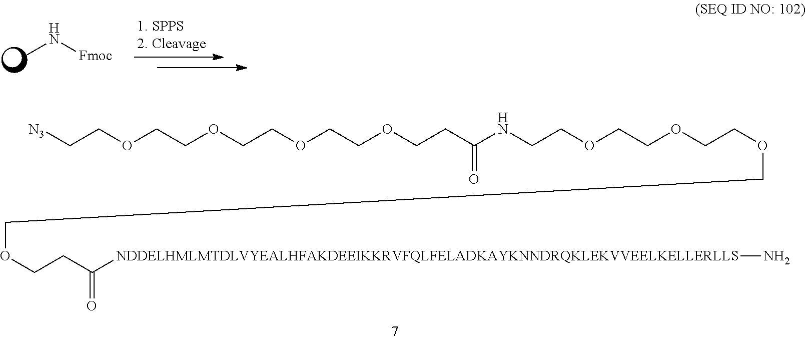

### Compound 7 was Synthesized Using Standard Fmoc Chemistry.

1) Resin preparation: DMF (10.0 mL) was added to the vessel containing Rink amide AM Resin (sub: 0.263 mmol/g, 0.1 mmol, 0.38 g) and swelled for 2 hrs.
2) Deprotection: 20% piperidine/DMF was added to the resin with N₂ bubbling for about 0.5 hr. The de-protection reaction was monitored by ninhydrin test, if it showed blue or other brownish red, the reaction was completed. The resin was then washed with DMF (20.0 mL)*5.
3) Coupling: Fmoc-amino acid solution in DMF (10.0 mL) was added to resin with N₂ bubbling, then activation buffer was added to the resin with N₂ bubbling for about 1 hr. The coupling reaction was monitored by ninhydrin test, if it showed blue, double coupling would be performed; if it showed colorless, the coupling was completed. The resin was then washed with DMF (20.0 mL)*5.
4) Step 2 and 3 were repeated for amino acids: (2-66 in table below), double coupling was performed for amino acids (Glu8, Arg20, Asp28, Phe35, Arg37, Glu42, His47, Asp54, His59, Asp63, and Asn64 in table below).
5) After the last N₃-PEG₈-CH₂CH₂COOH coupled, the resin was then washed with DMF (20.0 mL)*5, MeOH (20.0 mL)*5, and then dried under vacuum.

| # | Materials | Coupling reagents |
|---|---|---|
| 1 | Fmoc—Ser(tBu)—OH (3.00 eg) | HBTU (2.85 eg) and DIEA (6.00 eq) |
| 2 | Fmoc—Leu—OH (3.00 eg) | HBTU (2.85 eq) and DIEA (6.00 eq) |
| 3 | Fmoc—Leu—OH (3.00 eg) | HBTU (2.85 eg) and DIEA (6.00 eq) |
| 4 | Fmoc—Arg(Pbf)—OH (3.00 eg) | HBTU (2.85 eq) and DIEA (6.00 eq) |
| 5 | Fmoc—Glu(OtBu)—OH (3.00 eg) | HBTU (2.85 eq) and DIEA (6.00 eg) |
| 6 | Fmoc—Leu—OH (3.00 eg) | HBTU (2.85 eq) and DIEA (6.00 eg) |
| 7 | Fmoc—Leu—OH (3.00 eg) | HBTU (2.85 eq) and DIEA (6.00 eq) |
| 8 | Fmoc—Glu(OtBu)—OH (3.00 eg) | HBTU (2.85 eq) and DIEA (6.00 eg) |
| 9 | Fmoc—Lys(Boc)—OH (6.00 eq) | HATU (5.70 eq) and DIEA (12.00 eg) |
| 10 | Fmoc—Leu—OH (6.00 eq) | HATU (5.70 eq) and DIEA (12.00 eq) |
| 11 | Fmoc—Glu(OtBu)—OH (6.00 eq) | HATU (5.70 eq) and DIEA (12.00 eq) |
| 12 | Fmoc—Glu(OtBu)—OH (6.00 eq) | HATU (5.70 eg) and DIEA (12.00 eq) |
| 13 | Fmoc—Val—OH (6.00 eg) | HATU (5.70 eg) and DIEA (12.00 eg) |
| 14 | Fmoc—Val—OH (6.00 eg) | HATU (5.70 eq) and DIEA (12.00 eg) |
| 15 | Fmoc—Lys(Boc)—OH (6.00 eq) | HATU (5.70 eq) and DIEA (12.00 eq) |
| 16 | Fmoc—Glu(OtBu)—OH (6.00 eg) | HATU (5.70 eq) and DIEA (12.00 eq) |
| 17 | Fmoc—Leu—OH (6.00 eg) | HATU (5.70 eq) and DIEA (12.00 eq) |
| 18 | Fmoc—Lys(Boc)—OH (6.00 eg) | HATU (5.70 eq) and DIEA (12.00 eg) |
| 19 | Fmoc—Gln(Trt)—OH (6.00 eq) | HATU (5.70 eq) and DIEA (12.00 eg) |
| 20 | Fmoc—Arg(Pbf)—OH (6.00 eg) | HATU (5.70 eg) and DIEA (12.00 eq) |
| 21 | Fmoc—Asp(OtBu)—OH (6.00 eg) | HATU (5.70 eq) and DIEA (12.00 eq) |
| 22 | Fmoc—Asn(Trt)—OH (6.00 eg) | HATU (5.70 eq) and DIEA (12.00 eg) |
| 23 | Fmoc—Asn(Trt)—OH (6.00 eg) | HATU (5.70 eq) and DIEA (12.00 eq) |
| 24 | Fmoc—Lys(Boc)—OH (6.00 eg) | HATU (5.70 eq) and DIEA (12.00 eg) |
| 25 | Fmoc—Tyr(Bu)—OH (6.00 eq) | HATU (5.70 eq) and DIEA (12.00 eq) |
| 26 | Fmoc—Ala—OH (6.00 eg) | HATU (5.70 eq) and DIEA (12.00 eq) |
| 27 | Fmoc—Lys(Boc)—OH (6.00 eq) | HATU (5.70 eq) and DIEA (12.00 eq) |
| 28 | Fmoc—Asp(OtBu)—OH (6.00 eg) | HATU (5.70 eq) and DIEA (12.00 eq) |
| 29 | Fmoc—Ala—OH (6.00 eg) | HATU (5.70 eq) and DIEA (12.00 eq) |
| 30 | Fmoc—Leu—OH (6.00 eq) | HATU (5.70 eq) and DIEA (12.00 eq) |
| 31 | Fmoc—Glu(OtBu)—OH (6.00 eg) | HATU (5.70 eq) and DIEA (12.00 eg) |
| 32 | Fmoc—Phe—OH (6.00 eq) | HATU (5.70 eq) and DIEA (12.00 eq) |
| 33 | Fmoc—Leu—OH (6.00 eg) | HATU (5.70 eq) and DIEA (12.00 eq) |
| 34 | Fmoc—Gln(Trt)—OH (6.00 eq) | HATU (5.70 eq) and DIEA (12.00 eq) |
| 35 | Fmoc—Phe—OH (6.00 eg) | HATU (5.70 eq) and DIEA (12.00 eq) |
| 36 | Fmoc—Val—OH (6.00 eg) | HATU (5.70 eq) and DIEA (12.00 eg) |
| 37 | Fmoc—Arg(Pbf)—OH (6.00 eq) | HATU (5.70 eq) and DIEA (12.00 eq) |
| 38 | Fmoc—Lys(Boc)—OH (6.00 eg) | HATU (5.70 eq) and DIEA (12.00 eq) |
| 39 | Fmoc—Lys(Boc)—OH (6.00 eq) | HATU (5.70 eq) and DIEA (12.00 eq) |
| 40 | Fmoc—Ile—OH (6.00 eq) | HATU (5.70 eq) and DIEA (12.00 eg) |
| 41 | Fmoc—Glu(OtBu)—OH (6.00 eq) | HATU (5.70 eq) and DIEA (12.00 eq) |
| 42 | Fmoc—Glu(OtBu)—OH (6.00 eg) | HATU (5.70 eq) and DIEA (12.00 eq) |
| 43 | Fmoc—Asp(OtBu)—OH (6.00 eg) | HATU (5.70 eq) and DIEA (12.00 eq) |
| 44 | Fmoc—Lys(Boc)—OH (6.00 eg) | HATU (5.70 eq) and DIEA (12.00 eq) |
| 45 | Fmoc—Ala—OH (6.00 eq) | HATU (5.70 eq) and DIEA (12.00 eg) |
| 46 | Fmoc—Phe—OH (6.00 eg) | HATU (5.70 eq) and DIEA (12.00 eg) |
| 47 | Fmoc—His(Trt)—OH (6.00 eg) | HATU (5.70 eq) and DIEA (12.00 eq) |
| 48 | Fmoc—Leu—OH (6.00 eq) | HATU (5.70 eq) and DIEA (12.00 eq) |
| 49 | Fmoc—Ala—OH (6.00 eg) | HATU (5.70 eq) and DIEA (12.00 eg) |
| 50 | Fmoc—Glu(OtBu)—OH (6.00 eq) | HATU (5.70 eq) and DIEA (12.00 eq) |
| 51 | Fmoc—Tyr(Bu)—OH (6.00 eg) | HATU (5.70 eq) and DIEA (12.00 eg) |
| 52 | Fmoc—Val—OH (6.00 eq) | HATU (5.70 eq) and DIEA (12.00 eq) |
| 53 | Fmoc—Leu—OH (6.00 eg) | HATU (5.70 eq) and DIEA (12.00 eg) |
| 54 | Fmoc—Asp(OtBu)—OH (6.00 eg) | HATU (5.70 eq) and DIEA (12.00 eq) |
| 55 | Fmoc—Thr(tBu)—OH (6.00 eg) | HATU (5.70 eq) and DIEA (12.00 eg) |
| 56 | Fmoc—Met—OH (6.00 eq) | HATU (5.70 eq) and DIEA (12.00 eg) |
| 57 | Fmoc—Leu—OH (6.00 eg) | HATU (5.70 eq) and DIEA (12.00 eq) |
| 58 | Fmoc—Met—OH (6.00 eq) | HATU (5.70 eq) and DIEA (12.00 eg) |
| 59 | Fmoc—His(Trt)—OH (6.00 eg) | HATU (5.70 eq) and DIEA (12.00 eq) |
| 60 | Fmoc—Leu—OH (6.00 eg) | HATU (5.70 eg) and DIEA (12.00 eg) |
| 61 | Fmoc—Glu(OtBu)—OH (6.00 eq) | HATU (5.70 eq) and DIEA (12.00 eq) |
| 62 | Fmoc—Asp(OtBu)—OH (6.00 eg) | HATU (5.70 eq) and DIEA (12.00 eq) |
| 63 | Fmoc—Asp(OtBu)—OH (6.00 eq) | HATU (5.70 eq) and DIEA (12.00 eq) |
| 64 | Fmoc—Asn(Trt)—OH (6.00 eg) | HATU (5.70 eq) and DIEA (12.00 eq) |
| 65 | FmocNH—PEG₈—CH₂CH₂COOH (3.00 eq) | HATU (2.85 eq) and DIEA (6.00 eq) |
| 66 | N₃—PEG₈—CH₂CH₂COOH (3.00 eq) | HATU (2.85 eq) and DIEA (6.00 eq) |

### Peptide Cleavage and Purification:

1) Cleavage buffer (95% TFA/2.5% TIS/2.5% H₂O, 30.0 mL) was added to the flask containing the side chain protected peptide resin at room temperature and stirred for 2 hrs.
2) The filtrate was collected.
3) The peptide was precipitated with cold isopropyl ether and centrifuged (3 mins at 3000 rpm).
4) Isopropyl ether washed two additional times.
5) The crude peptide was dried under vacuum 2 hrs.
6) The crude peptide was purified by Prep_HPLC (A: 0.075% TFA in H₂O, B: ACN), followed by lyophilization to give compound 7 (55.0 mg, 90.0% purity, 6.66% yield, TFA salt) as a white solid.

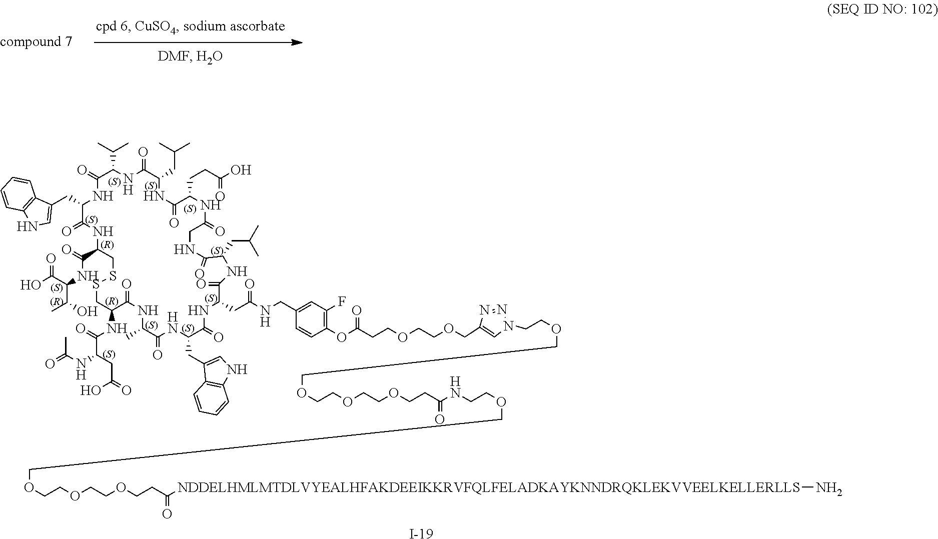

To a mixture of compound 7 (55.0 mg, 6.00 umol, 90.9% purity, 1.00 eq) and compound 6 (18.2 mg, 8.99 umol, 90.0% purity, 1.50 eq) in DMF (0.50 mL) was added a mixture of CuSO₄ (0.40 M, 14.9 uL, 1.00 eq), sodium ascorbate (4.75 mg, 23.98 umol, 4 eq), and THPTA (6.1 mg, 14.09 umol, 2.00 eq) in NH₄HCO₃ (0.2 M, 89.93 uL, 3.00 eq) at 15° C. under N₂ atmosphere. Then the mixture was stirred at 15° C. for 1 hr under N₂ atmosphere. The mixture was purified by prep-HPLC (acid condition, TFA) to afford compound I-19 (1.2 mg, 0.10 umol, 86.4% purity, 1.71% yield, TFA salt) (6.5 mg, 0.55 umol, 89.2% purity, 9.59% yield, TFA salt) as a white solid.

### Purification Conditions:

| | Separation condition |
|---|---|
| Dissolution | Dissolve in ACN/H₂O |
| condition | |
| Instrument | GX-281 |
| Mobile Phase | A: H₂O (TFA 0.075%) |
| | B: CH₃CN |
| Gradient | 25-55%-51 min. Retention time: 30 min |
| Column | Luna 100*25 mm, C18, 10 um, 100Å + Gemini ® |
| | 150*30 mm, C18, 5 um, 110Å |
| Flow Rate | 20 mL/min |
| Wavelength | 220/254 nm |
| Oven Temp. | Room temperature |

### Example 17. Certain Technologies for Preparing Agents: I-20, I-21, I-22 and I-23

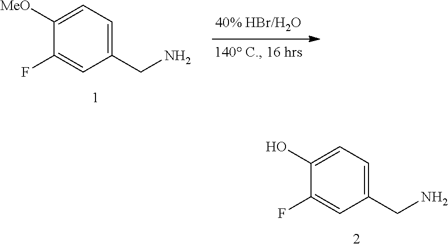 [SMILES: COc1ccc(CN)cc1F ; NCc1ccc(O)c(F)c1]

A mixture of compound 1 (75.0 g, 64.4 mmol) in HBr/H₂O (40% HBr, 1000 mL in total) was stirred at 140° C. for 16 hrs. The solvent was removed at 70° C. under reduced pressure, the residue was triturated in MeCN (50 mL) for 10 mins. After filtration, the solid was dried under lyophilization to get compound 2 (100.0 g, 427.9 mmol, 88.4% yield, HBr salt) as a brown solid. ¹H NMR: (400 MHz DMSO-d₆) δ ppm 10.03 (s, 1H) 8.20 (s, 3H) 7.32 (dd, J=12.17, 1.88 Hz, 1H) 7.11 (dd, J=8.28, 1.51 Hz, 1H) 6.95-7.03 (m, 1H) 3.93 (q, J=5.27 Hz, 2H).

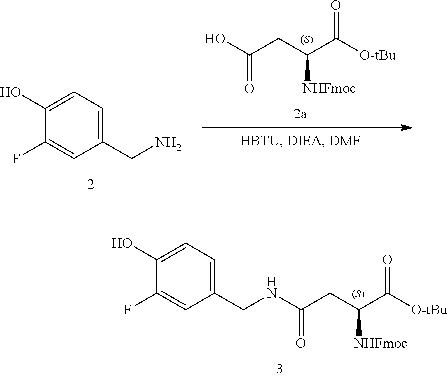

To a mixture of compound 2 (60.0 g, 270.2 mmol, 1.00 eq, HBr), compound 2a (111.1 g, 270.2 mmol, 1.00 eq), DIEA (34.9 g, 270.2 mmol, 47.0 mL, 1.00 eq) and HOBt (54.7 g, 405.3 mmol, 1.50 eq) in DMF (1000 mL) was added EDCI (56.7 g, 297.2 mmol, 1.10 eq) at 15° C., the mixture was stirred at 15° C. for 3 hrs. The mixture was dropwise added to 0.5 M HCl (cold, 10 L) and white solid was precipitated. After filtration, the solid was dissolved in DCM (2000 mL), washed with 0.5 M HCl (800 mL), H₂O (800 mL), brine (800 mL), dried over anhydrous Na₂SO₄ and concentrated under reduced pressure. The residue was purified by silica gel column (DCM:MeOH=20:1) to afford compound 3 (120.0 g, 90% purity, containing a little DMF, 83.3% yield) as a white solid. H NMR: (400 MHz DMSO-d₆) δ ppm 9.70 (s, 1H) 8.34 (t, J=5.77 Hz, 1H) 7.90 (d, J=7.53 Hz, 2H) 7.71 (d, J=7.53 Hz, 2H) 7.61 (d, J=8.28 Hz, 1H) 7.39-7.47 (m, 2H) 7.29-7.36 (m, 2H) 7.02 (d, J=12.30 Hz, 1H) 6.85-6.92 (m, 2H) 4.20-4.39 (m, 4H) 4.11-4.19 (m, 2H) 1.36 (s, 9H).

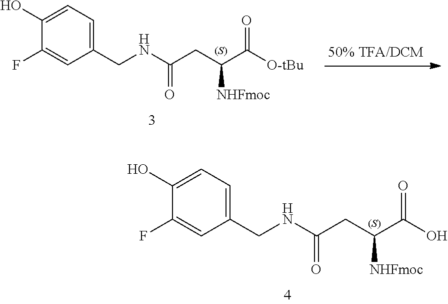

A mixture of compound 3 (120.0 g, 224.4 mmol, 1.00 eq) in TFA (600 mL) and DCM (600 mL) was stirred at 15° C. for 0.5 hr. The solvent was removed under reduced pressure. The residue was purified by silica gel column (DCM:MeOH=10:1) to get compound 4 (100 g, 93.5% purity, 93.1% yield) as a white solid. ¹H NMR: (400 MHz DMSO-d₆) δ ppm 9.69 (s, 1H) 8.34 (t, J=5.90 Hz, 1H) 7.90 (d, J=7.28 Hz, 2H) 7.71 (d, J=7.53 Hz, 2H) 7.54 (d, J=6.53 Hz, 1H) 7.42 (t, J=7.40 Hz, 2H) 7.27-7.37 (m, 1H) 7.27-7.37 (m, 1H) 7.02 (d, J=12.05 Hz, 1H) 6.82-6.93 (m, 2H) 4.35-4.43 (m, 1H) 4.20-4.31 (m, 3H) 4.13-4.19 (m, 2H).

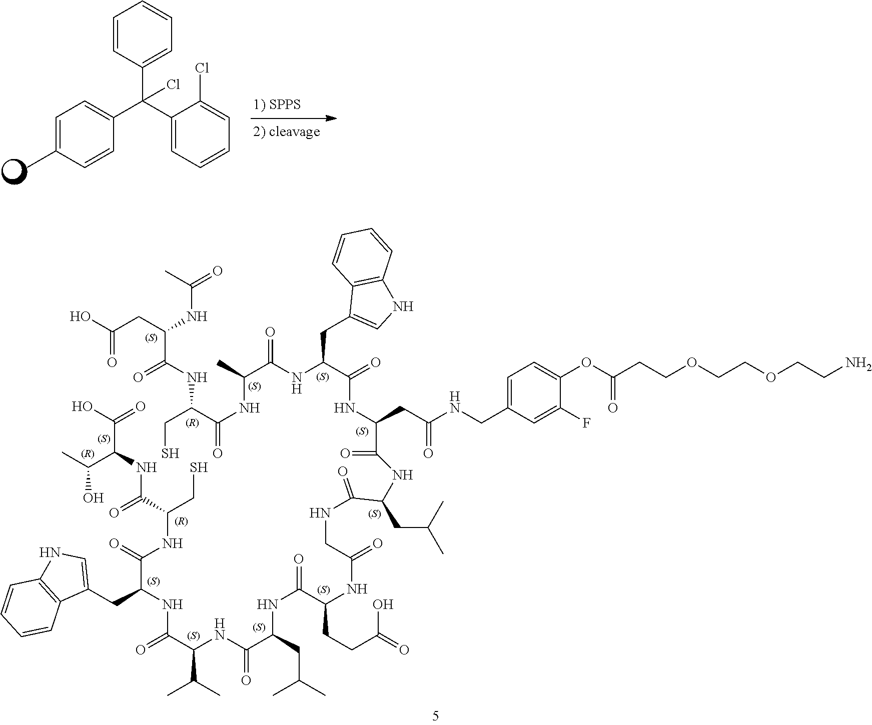

Compound 5 was synthesized using standard Fmoc chemistry.
1) Resin preparation: To the vessel containing CTC Resin (100.0 mmol, 100.0 g, 1.00 mmol/g) and Fmoc-Thr(tBu)-OH (39.7 g, 100.0 mmol, 1.00 eq) in DCM (1.5 L) was added DIEA (4.00 eq) dropwise and mixed for 2 hrs with N₂ bubbling at 15° C. Then MeOH (100.0 mL) was added and bubbled with N₂ for another 30 mins. The resin was washed with DMF (3.0 L)*5, followed by adding 20% piperidine in DMF (3.0 L) and bubbled with N₂ for 30 mins at 15° C. for Fmoc deprotection. The mixture was filtered and the resin was washed with DMF (2.0 L)*5 before proceeding to next step.
2) Coupling: A solution of Fmoc-Cys(Trt)-OH (175.5 g, 3.00 eq), HBTU (109.7 g, 2.85 eq) in DMF (1.5 L) was added to the resin with N₂ bubbling. Then DIEA (6.00 eq) was added to the mixture dropwise and bubbled with N₂ for 30 mins at 15° C. The coupling reaction was monitored by ninhydrin test, if the resin showed blue or brownish red, double coupling should be performed; if it showed colorless, the coupling was completed. The resin was then washed with DMF (3.0 L)*5.
3) Deprotection: 20% piperidine in DMF (3.0 L) was added to the resin and the mixture was bubbled with N₂ for 30 mins at 15° C. The deprotection reaction was monitored by ninhydrin test, if it showed blue or other brownish red, the reaction was completed. The resin was then washed with DMF (3.0 L)*5.
4) Step 2 and 3 were repeated for amino acids: (2-8 in table below).
5) Coupling: A solution of compound 4 (71.7 g, 1.50 eq), DIC (18.9 g, 1.50 eq) and HOBt (20.2 g, 1.50 eq) in DMF (1.2 L) was added to the resin with N₂ bubbling for 60 mins at 15° C. The coupling reaction was monitored by ninhydrin test, if it showed colorless, the coupling was completed. The resin was then washed with DMF (3.0 L)*5.
6) Step 3 for Fmoc deprotection.
7) Step 5 and 6 were repeated for amino acids (10-13 in table below).
8) Acetylation: A solution of 10% Ac₂O/5% NMM/85% DMF (1.5 L) was added to resin and the mixture was bubbled with N₂ for 20 mins. The acetylation reaction was monitored by ninhydrin test, if it showed colorless, the coupling was completed. The resin was then washed with DMF (3.0 L)*5.
9) 3% hydrazine in DMF (3.0 L) was added to resin with N₂ bubbling for 15 min to liberate hydroxyl group on compound 4, the resin was then washed with DMF (3.0 L)*5.
10) Modification on hydroxyl group on compound 4: A solution of Boc-NH-PEG₂-CH₂CH₂COOH (55.5 g, 200.0 mmol, 2.00 eq), DIC (2.00 eq), HOBt (2.00 eq) and DMAP (2.00 eq) in DMF (1.2 L) was added to resin and the mixture was bubbled with N₂ for 19 hrs. The coupling reaction was monitored by LCMS after a mini-cleavage, the main peak was desired MS. The resin was then washed with DMF (3.0 L)*5, MeOH (3.0 L)*5, and then dried under vacuum to afford peptide resin 340.0 g.

| # | Materials | Coupling reagents |
|---|---|---|
| 1 | Fmoc—Thr(tBu)—OH (1.00 eg) | DIEA (4.00 eg) |
| 2 | Fmoc—Cys(Trt)—OH (3.00 eg) | HBTU (2.85 eq) and DIEA (6.00 eq) |
| 3 | Fmoc—Trp—OH (3.00 eq) | HBTU (2.85 eq) and DIEA (6.00 eq) |
| 4 | Fmoc—Val—OH (3.00 eq) | HBTU (2.85 eq) and DIEA (6.00 eq) |
| 5 | Fmoc—Leu—OH (3.00 eq) | HBTU (2.85 eq) and DIEA (6.00 eq) |
| 6 | Fmoc—Glu(OtBu)—OH (3.00 eg) | HBTU (2.85 eq) and DIEA (6.00 eq) |
| 7 | Fmoc—Gly—OH (3.00 eg) | HBTU (2.85 eq) and DIEA (6.00 eg) |
| 8 | Fmoc—Leu—OH (3.00 eq) | HBTU (2.85 eq) and DIEA (6.00 eq) |
| 9 | compound 4 (1.50 eq) | DIC (1.50 eq) and HOBt (1.50 eq) |
| 10 | Fmoc—Trp—OH (3.00 eg) | DIC (3.00 eq) and HOBt (3.00 eg) |
| 11 | Fmoc—Ala—OH (3.00 eg) | DIC (3.00 eg) and HOBt (3.00 eg) |
| 12 | Fmoc—Cys(Trt)—OH (3.00 eq) | DIC (3.00 eq) and HOBt (3.00 eq) |
| 13 | Fmoc—Asp(OtBu)—OH (3.00 eq) | DIC (3.00 eg) and HOBt (3.00 eq) |
| 14 | Acetylation | Ac₂O/NMM/DMF (10/5/85, 1.5 L) |
| 15 | 3-(2-(prop-2-yn-1-yloxy)ethoxy)propanoic | DIC (2.00 eq), HOBt (2.00 eq) and |
| | acid (2.00 eq) | DMAP (2.00 eg) |

### Peptide Cleavage and Purification:

1) Cleavage buffer (95% TFA/2.5% TIS/2.5% H₂O, 3.0 L) was added to the flask containing the side chain protected peptide resin at room temperature and stirred for 1 hr.
2) The filtrate was collected.
3) The peptide was precipitated with cold isopropyl ether (20.0 L). After filtration, the crude peptide was washed with cold isopropyl ether (5.0 L)*2, and the solid was dried under vacuum for 2 hrs.
4) Compound 5 (152.5 g, crude) was obtained as a white solid.

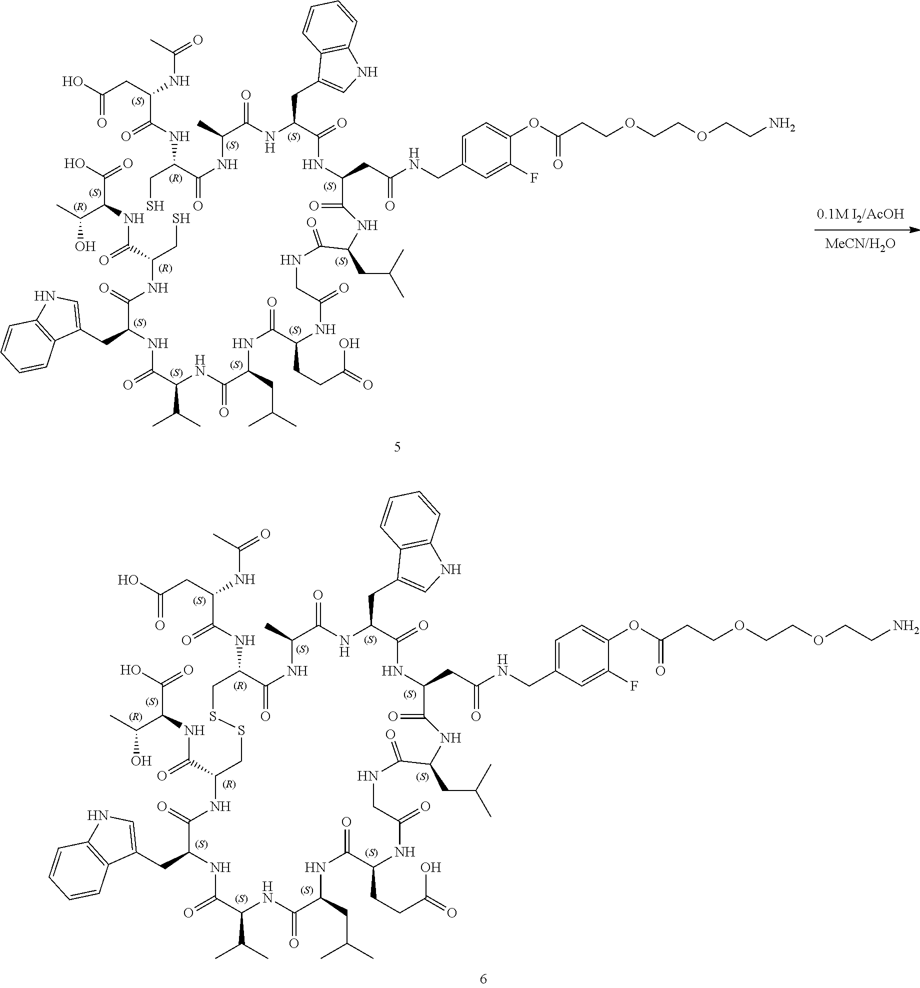

To a mixture of compound 5 (10.0 g, crude) in TFA/MeCN/H₂O (1/3/6, 5 L) was added 0.1 M I₂/HOAc dropwise until the yellow color persisted, then the mixture was stirred at 15° C. for 5 mins. The mixture was quenched with 0.1 M Na₂S₂O₃ dropwise until yellow color disappeared. The mixture was filtered, and the filtrate was purified by prep-HPLC (acid condition, TFA) directly, followed by lyophilization to afford compound 6 (24.8 g, 93.9% purity, 15.3% yield, 15 injection combined) as a white solid.

### Compound 7 was Synthesized Using Standard Fmoc Chemistry.

1) Resin preparation: To the vessel containing CTC Resin (0.30 mmol, 0.38 g, 0.78 mmol/g) and Fmoc-NH-PEG₈-CH₂CH₂COOH (159.1 mg, 0.24 mmol, 0.80 eq) in DCM (5.0 mL) was added DIEA (4.00 eq) dropwise and mixed for 2 hrs with N₂ bubbling at 15° C. Then MeOH (0.50 mL) was added and bubbled with N₂ for another 30 mins. The resin was washed with DMF (20.0 mL)*5, followed by adding 20% piperidine in DMF (20.0 mL) and bubbled with N₂ for 30 mins at 15° C. for Fmoc de-protection. The mixture was filtered and the resin was washed with DMF (20.0 mL)*5 before proceeding to next step.
2) Coupling: A solution of Fmoc-NH-PEG₈-CH₂CH₂COOH (318.2 mg, 0.60 mmol, 2.00 eq), HBTU (219.4 mg, 1.90 eq) in DMF (5.0 mL) was added to the resin with N₂ bubbling. Then DIEA (4.00 eq) was added to the mixture dropwise and bubbled with N₂ for 30 mins at 15° C. The coupling reaction was monitored by ninhydrin test, if resin showed blue or brownish red, double coupling should be performed; if it showed colorless, the coupling was completed. The resin was then washed with DMF (20.0 mL)*5.
3) Deprotection: 20% piperidine in DMF (20.0 mL) was added to the resin and the mixture was bubbled with N₂ for 30 mins at 15° C. The deprotection reaction was monitored by ninhydrin test, if it showed blue or other brownish red, the reaction was completed. The resin was then washed with DMF (20.0 mL)*5.
4) Step 2 and 3 were repeated for amino acids: (2-57 in table below), double coupling was performed for amino acids (Leu8, Asp19, Lys21, Met23, Leu28, His38, Glu41, Met46, Gln52 in table below).
5) Acetylation: A solution of 10% Ac₂O/5% NMM/85% DMF (20.0 mL) was added to resin and the mixture was bubbled with N₂ for 20 mins. The acetylation reaction was monitored by ninhydrin test, if it showed colorless, the coupling was completed. The resin was then washed with DMF (20.0 mL)*5 MeOH (20.0 mL), and dried under vacuum.

| # | Materials | Coupling reagents |
|---|---|---|
| 1 | Fmoc—NH—PEG₈—CH₂CH₂COOH (0.80 eq) | DIEA (4.00 eq) |
| 2 | Fmoc—NH—PEG₈—CH₂CH₂COOH (2.00 eq) | HBTU (1.90 eq) and DIEA (4.00 eg) |
| 3 | Fmoc—Arg(Pbf)—OH (1.00 eq) | HATU (2.85 eq) and DIEA (6.00 eg) |
| 4 | Fmoc—Glu(OtBu)—OH (3.00 eq) | HOBT (3.00 eq) and DIC (6.00 eq) |
| 5 | Fmoc—Val—OH (3.00 eq) | HBTU (2.85 eq) and DIEA (6.00 eq) |
| 6 | Fmoc—Glu(OtBu)—OH (3.00 eq) | HBTU (2.85 eq) and DIEA (6.00 eg) |
| 7 | Fmoc—Glu(OtBu)—OH (3.00 eq) | HBTU (2.85 eq) and DIEA (6.00 eg) |
| 8 | Fmoc—Leu—OH (3.00 eq) | HBTU (2.85 eq) and DIEA (6.00 eq) |
| 9 | Fmoc—Leu—OH (3.00 eq) | HBTU (2.85 eq) and DIEA (6.00 eq) |
| 10 | Fmoc—Arg(Pbf)—OH (3.00 eg) | HBTU (2.85 eq) and DIEA (6.00 eq) |
| 11 | Fmoc—Glu(OtBu)—OH (6.00 eg) | HBTU (2.85 eq) and DIEA (6.00 eq) |
| 12 | Fmoc—Ala—OH (6.00 eg) | HBTU (2.85 eq) and DIEA (6.00 eq) |
| 13 | Fmoc—Glu(OtBu)—OH (6.00 eq) | HBTU (2.85 eq) and DIEA (6.00 eq) |
| 14 | Fmoc—Glu(OtBu)—OH (6.00 eg) | HOBT (3.00 eq) and DIC (6-3.00 eg) |
| 15 | Fmoc—Leu—OH (6.00 eg) | HBTU (2.85 eq) and DIEA (6.00 eq) |
| 16 | Fmoc—Leu—OH (6.00 eg) | HBTU (2.85 eq) and DIEA (6.00 eg) |
| 17 | Fmoc—Arg(Pbf)—OH (6.00 eq) | HBTU (2.85 eq) and DIEA (6.00 eq) |
| 18 | Fmoc—Glu(OtBu)—OH (6.00 eg) | HBTU (2.85 eq) and DIEA (6.00 eq) |
| 19 | Fmoc—Asp(OtBu)—OH (6.00 eq) | HATU (2.85 eq) and DIEA (6.00 eq) |
| 20 | Fmoc—Gly—OH (6.00 eg) | HATU (2.85 eq) and DIEA (6.00 eq) |
| 21 | Fmoc—Lys(Alloc)—OH (6.00 eq) | HATU (2.85 eq) and DIEA (6.00 eq) |
| 22 | Fmoc—Lys(Alloc)—OH (6.00 eq) | HATU (2.85 eq) and DIEA (6.00 eq) |
| 23 | Fmoc—Met—OH (6.00 eg) | HATU (2.85 eq) and DIEA (6.00 eq) |
| 24 | Fmoc—Phe—OH (6.00 eq) | HATU (2.85 eg) and DIEA (6.00 eg) |
| 25 | Fmoc—Glu(OtBu)—OH (6.00 eg) | HATU (2.85 eq) and DIEA (6.00 eq) |
| 26 | Fmoc—Tyr(tBu)—OH (6.00 eg) | HATU (2.85 eq) and DIEA (6.00 eq) |
| 27 | Fmoc—Ile—OH (6.00 eq) | HOBT (3.00 eq) and DIC (6.00 eq) |
| 28 | Fmoc—Leu—OH (6.00 eg) | HATU (2.85 eq) and DIEA (6.00 eg) |
| 29 | Fmoc—Asp(OtBu)—OH (6.00 eq) | HATU (2.85 eq) and DIEA (6.00 eq) |
| 30 | Fmoc—Ser(OtBu)—OH (6.00 eg) | HATU (2.85 eq) and DIEA (6.00 eg) |
| 31 | Fmoc—Val—OH (6.00 eg) | HATU (2.85 eq) and DIEA (6.00 eg) |
| 32 | Fmoc—Arg(Pbf)—OH (6.00 eg) | HATU (2.85 eq) and DIEA (6.00 eq) |
| 33 | Fmoc—Met—OH (6.00 eg) | HATU (2.85 eq) and DIEA (6.00 eq) |
| 34 | Fmoc—Ser(tBu)—OH (6.00 eg) | HATU (2.85 eq) and DIEA (6.00 eq) |
| 35 | Fmoc—Ala—OH (6.00 eg) | HATU (2.85 eq) and DIEA (6.00 eg) |
| 36 | Fmoc—Glu(OtBu)—OH (6.00 eq) | HATU (2.85 eq) and DIEA (6.00 eq) |
| 37 | Fmoc—Ala—OH (6.00 eg) | HATU (2.85 eq) and DIEA (6.00 eg) |
| 38 | Fmoc—His(Trt)—OH (6.00 eg) | HATU (2.85 eq) and DIEA (6.00 eg) |
| 39 | Fmoc—Gly—OH (6.00 eg) | HATU (2.85 eq) and DIEA (6.00 eq) |
| 40 | Fmoc—Leu—OH (6.00 eg) | HATU (2.85 eq) and DIEA (6.00 eg) |
| 41 | Fmoc—Glu(OtBu)—OH (6.00 eg) | HATU (2.85 eq) and DIEA (6.00 eq) |
| 42 | Fmoc—Asp(OtBu)—OH (6.00 eq) | HATU (2.85 eq) and DIEA (6.00 eq) |
| 43 | Fmoc—Leu—OH (6.00 eg) | HATU (2.85 eq) and DIEA (6.00 eq) |
| 44 | Fmoc—Leu—OH (6.00 eq) | HATU (2.85 eq) and DIEA (6.00 eg) |
| 45 | Fmoc—Arg(Pbf)—OH (6.00 eq) | HATU (2.85 eq) and DIEA (6.00 eq) |
| 46 | Fmoc—Met—OH (6.00 eq) | HATU (2.85 eq) and DIEA (6.00 eq) |
| 47 | Fmoc—Ile—OH (6.00 eg) | HATU (2.85 eq) and DIEA (6.00 eq) |
| 48 | Fmoc—Glu(OtBu)—OH (6.00 eq) | HATU (2.85 eq) and DIEA (6.00 eg) |
| 49 | Fmoc—Tyr(tBu)—OH (6.00 eg) | HATU (2.85 eq) and DIEA (6.00 eq) |
| 50 | Fmoc—Ile—OH (6.00 eq) | HATU (2.85 eq) and DIEA (6.00 eq) |
| 51 | Fmoc—Lys(Alloc)—OH (6.00 eq) | HATU (2.85 eq) and DIEA (6.00 eq) |
| 52 | Fmoc—Gln(Trt)—OH (6.00 eg) | HATU (2.85 eq) and DIEA (6.00 eq) |
| 53 | Fmoc—Leu—OH (6.00 eq) | HATU (2.85 eq) and DIEA (6.00 eq) |
| 54 | Fmoc—Ile—OH (6.00 eg) | HATU (2.85 eq) and DIEA (6.00 eg) |
| 55 | Fmoc—Trp(Boc)—OH (6.00 eq) | HATU (2.85 eq) and DIEA (6.00 eq) |
| 56 | Fmoc—Glu(OtBu)—OH (6.00 eg) | HATU (2.85 eq) and DIEA (6.00 eg) |
| 57 | Fmoc—Lys(Alloc)—OH (6.00 eq) | HATU (2.85 eq) and DIEA (6.00 eg) |
| 58 | Fmoc—Asp(OtBu)—OH (6.00 eq) | HATU (2.85 eq) and DIEA (6.00 eq) |
| 59 | Acetylation | Ac₂O/NMM/DMF (10/5/85, 20.0 mL) |

### Peptide Cleavage and Purification:

1) Cleavage buffer (20% HFIP/DCM, 30.0 mL) was added into the flask containing the side chain protected crude peptide. The mixture was stirred for 0.5 hr at room temperature and the solution was collected after filtration. The cleavage step was repeated for another time.
2) The combined solution was concentrated by rotary evaporation.
3) The crude peptide was dried under lyophilization.
4) Compound 7 (3.0 g, crude) was obtained as a white solid.

To a mixture of compound 7 (1.5 g, 144.6 umol, 1.00 eq), TFP (192.1 mg, 1.16 mmol, 8.00 eq) and EDCI (110.9 mg, 578.5 umol, 4.00 eq) in DMF (20.0 mL) was stirred at 15° C. for 16 hrs. The mixture was added to 1 M HCl (cold, 200 mL) to precipitate out crude product. After filtration, the solid was collected to give compound 8 (theoretical amount<1.6 g, crude, wet solid), compound 8 is used for next step directly without further work-up or purification.

A mixture of compound 8 (theoretical amount<1.6 g, crude, wet solid) in TFA/TIS/H₂O (95/2.5/2.5, 30 mL) was stirred at 15° C. for 1 hr. The mixture was precipitated with cold isopropyl ether (300 mL), after filtration, the solid was washed with cold isopropyl ether (300 mL), followed by drying under vacuum for 2 hrs. The residue was purified by prep-HPLC (TFA condition) to get compound 9 (50.0 mg, 91.0% purity, 4.2% yield) as a white solid.

A mixture of compound 9 (50.0 mg, 6.41 umol, 1.00 eq) and compound 6 (14.10 mg, 7.69 umol, 1.20 eq), DIEA (8.2 mg, 64.0 umol, 11.1 uL, 10.00 eq) in DMF (0.6 mL) was stirred at 15° C. for 1 hr. The mixture was purified by prep-HPLC (acid condition, TFA) to afford compound 10 (40.0 mg, 4.06 umol, 65.9% yield) as a white solid.

A mixture of compound 10 (40.0 mg, 4.06 umol, 1.00 eq), Pd(PPh₃)₄ (2.35 mg, 2.03 umol, 0.50 eq), and phenylsilane (8.7 mg, 81.2 umol, 10.0 uL, 20.00 eq) in DMF (0.50 mL) was stirred at 15° C. for 1 hr under N₂ atmosphere. The mixture was purified by prep-HPLC (acid condition, TFA) to afford compound I-20 (23.1 mg, 2.40 umol, 59.0% yield, TFA salt) as a white solid.

### Purification Conditions:

Separation condition

| | |
|---|---|
| Dissolution | Dissolve in ACN/H₂O |
| condition | |
| Instrument | GX-281 |
| Mobile Phase | A: H₂O (TFA 0.075%) |
| | B: CH₃CN |
| Gradient | 30-60%-51 min. Retention time: 40 min |
| Column | Luna 100*25 mm, C18, 10 um, 100Å + Gemini ® |
| | 150*30 mm, C18, 5 um, 110Å |
| Flow Rate | 20 mL/min |
| Wavelength | 220/254 nm |
| Oven Temp. | Room temperature |

Compound I-21, I-22, and I-23 were synthesized via the same methodology. Compound I-23 used one Fmoc-PEG8-OH for SPPS. The last amino acid for compounds I-21 and I-22 is Alloc-Asp(OtBu)-OH. After fragments condensation, the N-terminal Alloc was removed to give free amine in the last de-Alloc step. For one preparation of I-21, 92% purity was observed. For one preparation of I-22, 92% purity was observed. For one preparation of I-23, 98% purity was observed. For each of I-20, I-21, I-22 and I-23, expected mass was observed in MS.

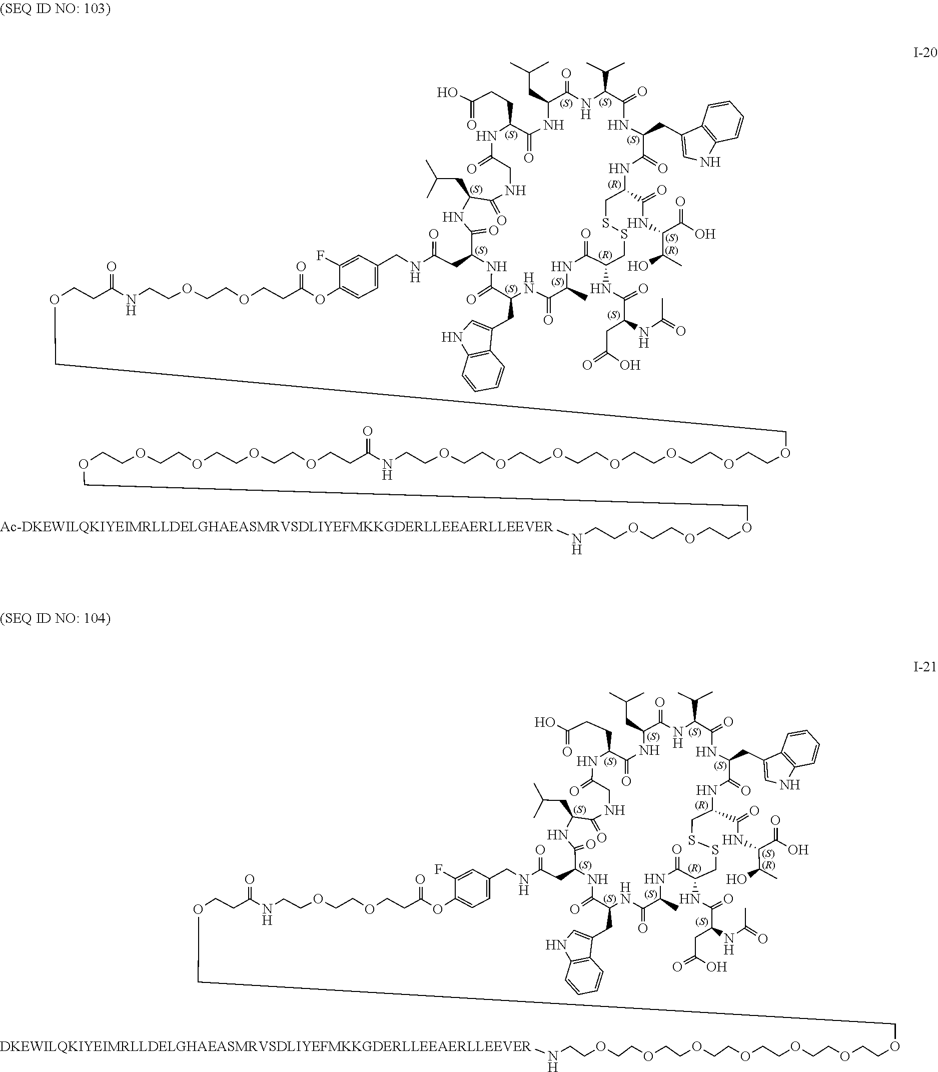

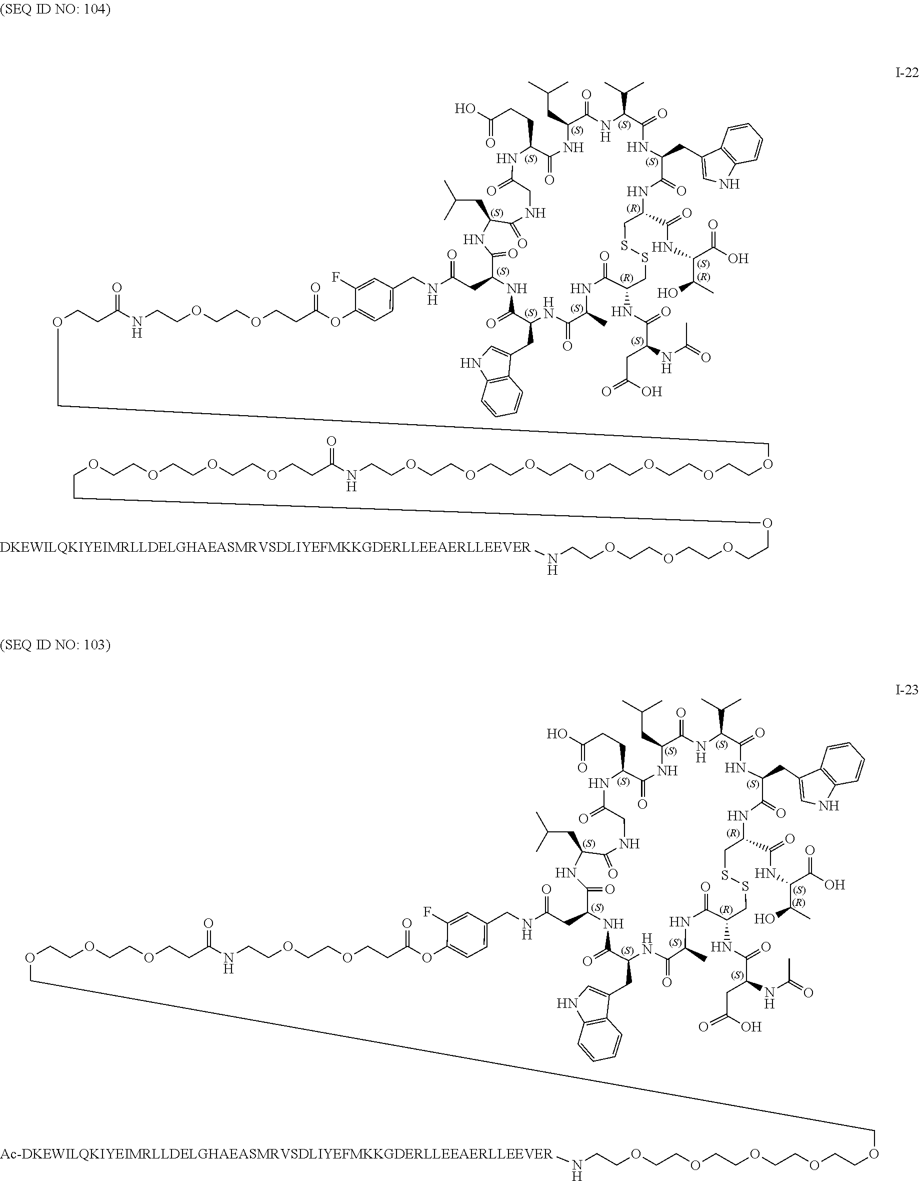

### Example 18. Certain Technologies for Preparing Agents: I-20, I-21, I-22 and I-23

In some embodiments, the present disclosure provides technologies for preparing MATE agents and compositions thereof. In some embodiments, provided technologies comprise reacting a composition comprising a plurality of antibody agents (e.g., IVIG compositions such as Gamunex-C) with a composition comprising a plurality of agents each comprising a target binding moiety, an antibody binding moiety, and a reactive group in between (and optional linker moieties linking such moieties) (e.g., I-3, I-7, I-8, I-15, I-19, I-20, I-21, I-22, I-23, etc.). Described below as examples are preparations of certain MATE agents and compositions thereof.

### 1. A Reaction/Conjugation Protocol.

### 1.1 IVIG Buffer Exchange

Gamunex-C (1 mL, ˜100 mg/mL) was buffer exchanged into 50 mM borate pH 8.2 using Amicon-15 mL unit with MWCO 30 kDa. The protein concentration of the buffer exchanged IVIG was determined by UV-Vis using extinction coefficient of 1.41 mL·mg⁻¹cm⁻¹ at 280 nm. The buffer exchanged IVIG was adjusted to 20 mg/mL using 50 mM borate buffer pH 8.2.

### 1.2 Reagent Stock Solution Preparation

Reagents were weighed individually and the DMSO volume used to prepare the stock solution is calculated as in the following:

$$DMSO volume ( mL ) = ( Solid weight ( mg ) / Molecular weight ) \times purity ( \% ) / 5 mM \times {10}^{6}$$

### Data for Certain Preparations of Reagents are Presented Below:

| | Solid | | | DMSO | Final |
|---|---|---|---|---|---|
| | amount | Purity | Molecular | volume | reagent |
| Reagent | (mg) | (%) | weight | (μL) | concentration |
| I-8 | 4.05 | 77.1 | 9159.31 | 68.2 | 5 mM |
| I-7 | 2.17 | 87.1 | 9511.73 | 39.7 | |
| I-15 | 1.85 | 86.0 | 9510.75 | 33.4 | |
| I-19 | 1.44 | 86.4 | 10084.38 | 24.7 | |
| I-21 | 0.65 | 60.3 | 9049.24 | 8.7 | |
| I-22 | 2.42 | 91.8 | 9472.74 | 46.9 | |
| I-23 | 1.50 | 91.5 | 9091.28 | 30.2 | |
| I-20 | 2.39 | 97.9 | 9514.78 | 49.2 | |

### 1.3 Conjugation Reaction Setup

Procedure for I-29, I-30, I-31, I-32, I-34, I-35, I-36: To IVIG in 50 mM borate pH 8.2 (4 mg, 20 mg/mL, 200 μL) was added reagent (5 mM in DMSO, 13.3 μL, 2.5 eq.). The reaction was mixed thoroughly and rotated at room temperature overnight. Conjugations of a MATE reagent with immunoglobin antibody can accomplished using a subcutaneous, IM injectable, or intravenous immunoglobin preparation. For example the immunoglobin can be subcutaneous Ig (IGSC, such as the human subcutaneous immunoglobin products, including the brands CUTAQUIG (16.5% Ig solution, from Octapharma, Lachen, Switzerland), HIZENTRA (human subcutaneous immunoglobin, 20% Ig solution from CSL Bering, King of Prussia, NJ, USA), and XEMBIFY (human subcutaneous immunoglobin, 20% Ig solution from Grifols, Barcelona, Spain) or intravenous Immunoglobin (IVIG, such as human intravenous immunoglobin products, including the brands BIVIGAM (human intravenous Ig, 10% solution, from ADMA Biologics, Ramsey NJ, USA) and GAMUNEX-C (human injectable Ig, 10% solution, from Grifols, Barcelona, Spain).

### Procedure for I-33:

To IVIG in 50 mM borate pH 8.2 (2.62 mg, 20 mg/mL, 130.8 μL) was added reagent (5 mM in DMSO, 8.7 μL, 2.5 eq.). The reaction was mixed thoroughly and rotated at room temperature overnight.

### 2. Purification

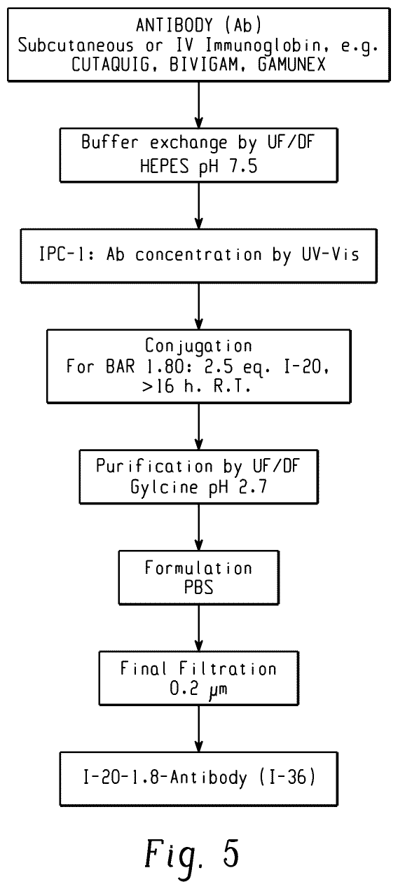

A crude mixture was buffer exchanged with 50 mM glycine pH 2.2 in Amicon-4 mL MWCO 30 kDa for 2 cycles. Then it was buffer exchanged into PBS in Amicon-4 mL MWCO 30 kDa for 2 cycles. Insoluble white precipitates were observed at the bottom of the filter. In some embodiments, released antibody binding moieties were removed under acidic conditions. Solution and precipitates from the eight samples were collected in 1.5 mL tube, respectively, and centrifuged at 16000 g for 5 min. The clear supernatant was carefully collected. The concentration of protein conjugates in supernatant was determined by UV-Vis Nanodrop instrument using extinction coefficient 1.41 mL·mg-1cm-1. A schematic showing the preparation of I-36 from MATE reagent I-20 is provided in FIG. 5. The conjugation and purification procedure is carried out as set forth for the preparation of I-33. The following preparation details should be noted. Following the addition of 2.5 equivalents of I-20 to immunoglobin (CUTAQUIG in the FIG. 5 schematic) the reaction solution is incubated for more than 16 hours at room temperature. The antibody concentration is 30 g/L. Purification is via ultrafiltration/diafiltration (UF/DF) in glycine, pH 2.7, followed by formulation in PBS, and a final filtration using a 0.2μ filter. The 2.5 MATE reagent (I-20) to antibody ratio is used to provide a final 1.8 binder to antibody ratio. The same procedure was also carried out using BIVIGAM and Gamunex as the Ig.

Concentration, volume, and yield of protein conjugates from certain preparations are shown in the table below.

| | Concentration | Volume | Product | Yield |
|---|---|---|---|---|
| Agent | (mg/mL) | (mL) | mass (mg) | (%) |
| I-29 | 6.29 | 0.501 | 3.15 | 79 |
| I-30 | 6.86 | 0.45 | 3.09 | 77 |
| I-31 | 2.25 | 0.667 | 1.5 | 38 |
| I-32 | 4.83 | 0.695 | 3.36 | 84 |
| I-33 | 5.55 | 0.548 | 3.04 | 116 |
| I-34 | 7.35 | 0.365 | 2.68 | 67 |
| I-35 | 10.7 | 0.326 | 3.49 | 87 |
| I-36 | 3.03 | 0.707 | 2.14 | 53 |

To adjust the final MATE agent concentration to 1.5-3.0 mg/mL range, all the conjugates were diluted with PBS to target for 3.0 mg/mL. In some embodiments, it was observed that, upon adding PBS, white precipitation quickly formed in some or all samples. To recover the conjugates, those samples were centrifuged at 16000 g for 3 minutes. The clear supernatants were carefully collected. The concentration of protein conjugates in supernatant was determined by UV-Vis Nanodrop instrument using extinction coefficient 1.41 mL·mg⁻¹cm⁻¹. The concentration, volume, and yield of the final conjugates from certain preparations are summarized in the table below.

| Agent | Concentration (mg/mL) | Volume (mL) | Product mass (mg) | Yield (%) |
|---|---|---|---|---|
| I-29 | 1.58 | 0.66 | 1.04 | 26 |
| I-30 | 1.52 | 0.92 | 1.39 | 35 |
| I-31 | 2.25 | 0.667 | 1.5 | 38 |
| I-32 | 2.15 | 1.00 | 2.15 | 54 |
| I-33 | 2.15 | 0.69 | 1.16 | 44 |
| I-34 | 1.62 | 0.79 | 1.28 | 27 |
| I-35 | 2.48 | 0.64 | 1.58 | 40 |
| I-36 | 3.03 | 0.707 | 2.14 | 53 |

### 3. Analytical Method for DAR (Ratio of Target Binding Moiety/Antibody Moiety) Determination

MATE conjugate (50 μg) was incubated with 10× glycol-buffer 2 (0.1× total volume), 1 μL IdeZ and 1 μL PNGase F at 37° C. for 1 hour. Then 8 μg of the sample was taken out and diluted 50× with water. The injection volume was 2 μL.

### Instrument:

LC: Waters Acquity UPLC protein BEH C4 column
MS: Waters Xevo G2-QTOF
Software: MassLynx
Mobile A: water+0.10% formic acid
Mobile B: acetonitrile+0.1% formic acid
Data analysis: deconvolute m/z 8000-30000, resolution 1.50. The results of DAR by LC-MS analysis are shown below.

| | DAR of crude | DAR of purified |
|---|---|---|
| Agent | conjugates | conjugates |
| I-29 | 1.08 | 1.47 |
| I-30 | 1.64 | 1.78 |
| I-31 | 1.75 | 1.72 |
| I-32 | 0.90 | 1.21 |
| I-33 | 1.81 | 1.73 |
| I-34 | 1.58 | 1.77 |
| I-35 | 1.76 | 1.76 |
| I-36 | 1.49 | 1.79 |

While we have described a number of embodiments, it is apparent that our basic examples may be altered to provide other embodiments that technologies (e.g., compounds, agents, compositions, methods, etc.) of the present disclosure. Therefore, it will be appreciated that the scope of an invention is to be defined by claims rather than by the specific embodiments that have been represented by way of example.

1. An agent comprising:
an antibody moiety or antibody binding moiety,
a target binding moiety, and
optionally a linker moiety linking the antibody moiety and the target binding moiety,
wherein the agent has the structure of formula M-II: 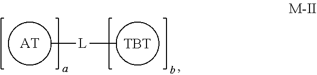
or a pharmaceutically acceptable salt thereof, wherein:
each of a and b is 1,
each AT is an antibody moiety or antibody binding moiety;
L is a linker moiety, and
the antibody moiety or antibody binding moiety comprises
(i) an antibody or a fragment thereof from an IVIG preparation; or
(ii) 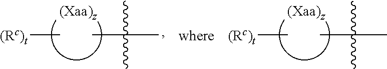
is chosen from A-1 to A-50 as shown in Table A-1.

2. (canceled)

3. The agent of claim 1, wherein the antibody moiety comprises IgG1 or a fragment thereof, IgG2 or a fragment thereof, or IgG4 or a fragment thereof.

4. The agent of claim 3, wherein the antibody moiety comprises IgG1 or a fragment thereof linked to the linker L an amino acid residue selected from K246 and K248 of an IgG1 heavy chain and amino acid residues corresponding thereto; or
the antibody moiety comprises IgG2 or a fragment thereof IgG2 or a fragment thereof is linked to the linker, at an amino acid residue selected from K251 and K253 of an IgG2 heavy chain and amino acid residues corresponding thereto; or
the antibody moiety comprises IgG4 or a fragment thereof is linked to the linker, at an amino acid residue selected from K239 and K241 of an IgG4 heavy chain and amino acid residues corresponding thereto.

5. The agent of claim 4, wherein L is a covalent bond, or a bivalent or polyvalent optionally substituted, linear or branched C₁₋₁₀₀ group comprising one or more aliphatic, aryl, heteroaromatic having 1-20 heteroatoms, or any combinations thereof, wherein one or more methylene units of the group are optionally and independently replaced with C₁₋₆ alkylene, C₁₋₆ alkenylene, —C≡C—, -Cy-, —C(R′)₂—, —O—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or —[(—O—C(R′)₂—C(R′)₂—)ₙ]—, wherein n is 1-20;
Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
each R′ is independently —R, —C(O)R, —CO₂R, or —SO₂R;
each R is independently —H, or an optionally substituted.

6. The agent of claim 4, wherein the linker comprises one or more —[(CH₂)n-O]m-, wherein each n is independently 1-20, and m is 1-100.

7. The agent of claim 6, additionally comprising a reactive group, RG, bound to the linker group, where RG is a group of the formula -L^{LG2}, -L^{LG2}-L^{LG3}-L^{LG4}-L^{RG1}- or -L^{RG1}-L^{RG2}-, where
L^{LG2} is —NH—, —NHC(O)—, —(CH₂)n-NHC(O)—, —(CH₂)n-OC(O)—, —(CH₂)n-OC(O)NH—, —C(O)—NHCH₂—, —C(O)—NHCH₂CH₂—, —C(O)O—CH₂—, or NH—C(O)O—CH₂—;
L^{LG3} is an optionally substituted aryl ring;
L^{LG4-} is a bond, —NH— or —O—;
L^{RG1} is —O—C(O)—, —CO)—, —S(O)— —OS(O)₂—, or —OP(O(OR)—;
L^{RG2} is —CH₂—C(O)—, —C(O)— or —CH₂—;
L^{LG} is —(O)C—[(CH₂)nO]m(CH₂)nNH—, —(O)C—[(CH₂)nO]m(CH₂)nNH—, —[(CH₂)nO]mNHC(O)[(CH₂)nO]mNH—, —[(CH₂)nO]m{NHC(O)[(CH₂)nO]m}pNH—, —[(CH₂)nO]mCy [(CH₂)nO]mNH, —[(CH₂)nO]mCy [(CH₂)nO]mNH C(O)[(CH₂)nO]mNH—, or —[(CH₂)nO]mCy [(CH₂)nO]m{NH C(O)[(CH₂)nO]m}pNH—,
where n, m, and p are integers independently chosen at each occurrence from 1-12, and Cy is an optionally substituted cyclic group.

8. The agent of claim 7, wherein RG is a group of the formula -L^{LG2}-L^{LG3}-L^{LG4}-L^{RG1} and is selected from:

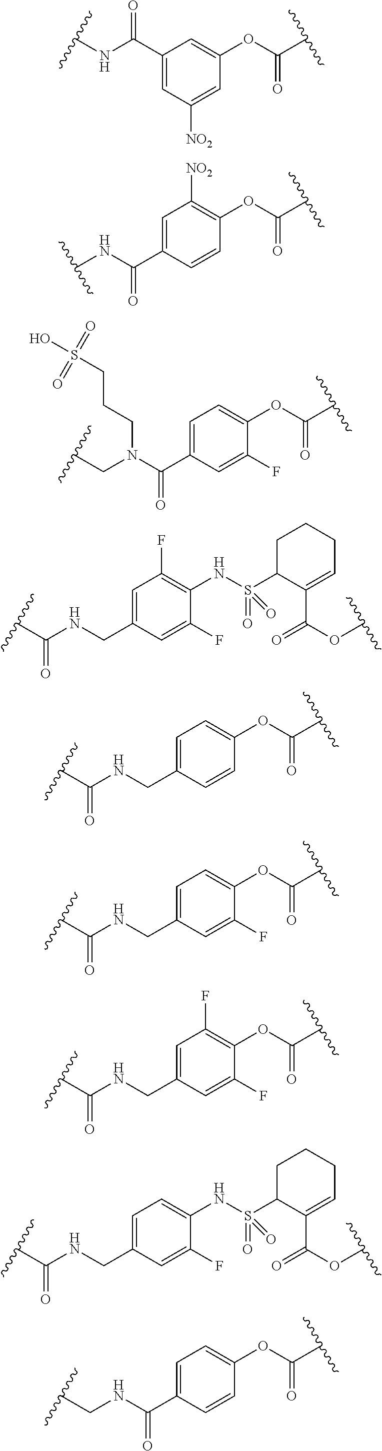

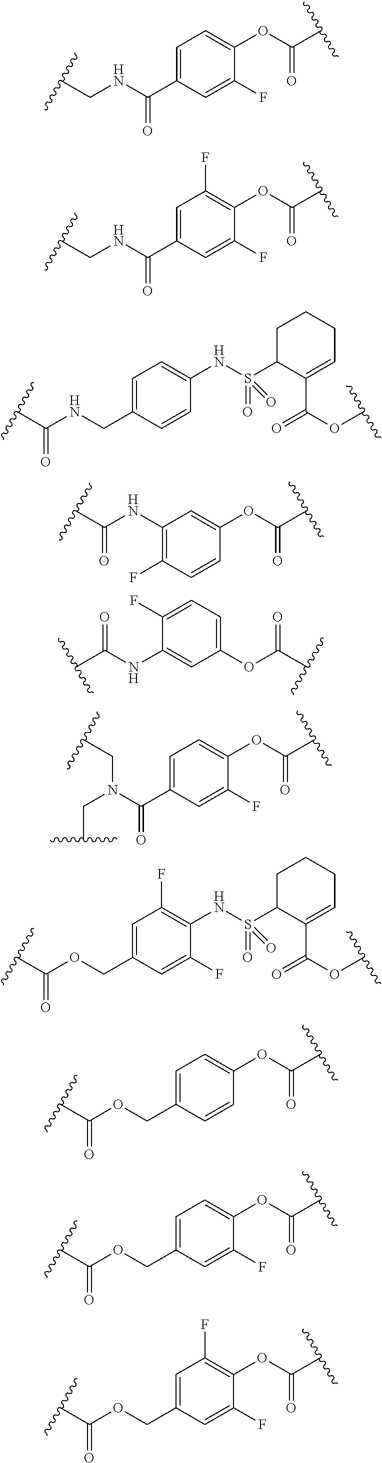

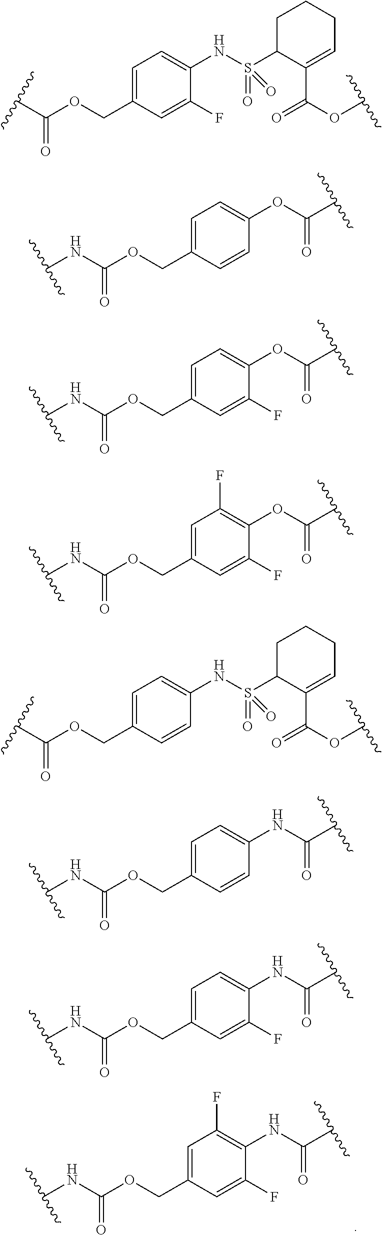

9-12. (canceled)

13. The agent of comprises a reactive group, wherein upon contact with an antibody, the reactive group reacts with a group of the antibody and conjugates a target binding moiety, or a moiety comprising -(Xaa)y-, to the antibody optionally through a linker, where -(Xaa)y is a peptide having at least 90% sequence homology to one of the following sequences:
(SEQ ID NO: 1)
DEDLEELERLYRKAEEVAKEAKDASRRGDDERAKEQMERAMRLFDQVFE
LAQELQEKQTDGNRQKATHLDKAVKEAADELYQRVR,
(SEQ ID NO: 2)
ELEEQVMHVLDQVSELAHELLHKLTGEELERAAYFNWWATEMMLELIKS
DDEREIREIEEEARRILEHLEELARK,
(SEQ ID NO: 3)
DKEWILQKIYEIMRLLDELGHAEASMRVSDLIYEFMKKGDERLLEEAER
LLEEVER,
(SEQ ID NO: 4)
DKEEILNKIYEIMRLLDELGNAEASMRVSDLILEFMKKGDERLLEEAER
LLEEVER,
(SEQ ID NO: 5)
SDDEDSVRYLLYMAELRYEQGNPEKAKKILEMAEFIAKRNNNEELERLV
REVKKRL,
(SEQ ID NO: 6)
NDDELHMLMTDLVYEALHFAKDEEIKKRVFQLFELADKAYKNNDRQKLE
KVVEELKELLERLLS,
(SEQ ID NO: 7)
QREKRLKQLEMLLEYAIERNDPYLMFDVAVEMLRLAEENNDERIIERAK
RILEEYE,
(SEQ ID NO: 8)
SLEELKEQVKELKKELSPEMRRLIEEALRFLEEGNPAMAMMVLSDLVYQ
LGDPRVIDLYMLVTKT,
(SEQ ID NO: 9)
DREQRLVRFLVRLASKFNLSPEQILQLFEVLEELLERGVSEEEIRKQLE
EVAKELG,
(SEQ ID NO: 10)
DDDIRYLIYMAKLRLEQGNPEEAEKVLEMARFLAERLGMEELLKEVREL
LRKIEELR,
and
(SEQ ID NO: 11)
PIIELLREAKEKNDEFAISDALYLVNELLQRTGDPRLEEVLYLIWRALK
EKDPRLLDRAIELFER.

14. (canceled)

15. The agent of claim 7, wherein the reactive group is or comprises 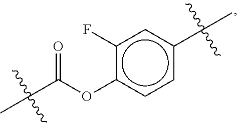
wherein —C(O)— is connected to a target binding moiety, or a moiety comprising -(Xaa)y-, optionally through a linker.

16. (canceled)

17. An agent, wherein the agent has the structure of formula R-I:
LG-RG-L^{RM}-MOI, (R-I)
or a salt thereof, wherein:
LG is R^{LG}-L^{LG};
R^{LG} is 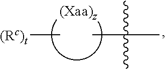
R^{c}-(Xaa)z-, IVIG, a nucleic acid moiety, or a small molecule moiety;
each Xaa is independently a residue of an amino acid or an amino acid analog;
t is 0-50;
z is 1-50;
each R^{c} is independently -L^{a}-R′;
each of a and b is independently 1-200;
each L^{a} is independently a covalent bond, or an optionally substituted bivalent group selected from C₁-C₂₀ aliphatic or C₁-C₂₀ heteroaliphatic having 1-5 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent monocyclic, bicyclic or polycyclic group wherein each monocyclic ring is independently selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
L^{LG} is -L^{LG1}-, -L^{LG1}-L^{LG2}-, -L^{LG1}-L^{LG2}-L^{LG3}-, or -L^{LG1}-L^{LG2}-L^{LG3}-L^{LG4}-;
RG is -L^{RG1}-L^{RG2}-, -L^{LG4}-L^{RG1}-L^{RG2}-, -L^{LG3}-L^{LG4}-L^{RG1}-L^{RG2}-, -L^{LG2}-L^{LG3}-L^{LG4}-L^{RG1}-L^{RG2}-;
each of L^{LG1}, L^{LG2}, L^{LG3}, L^{LG4}, L^{RG1}, L^{RG2}, and L^{RM} is independently L;
each L is independently a covalent bond, or a bivalent optionally substituted, linear or branched C₁₋₁₀₀ group comprising one or more aliphatic moieties, aryl moieties, heteroaliphatic moieties each independently having 1-20 heteroatoms, heteroaromatic moieties each independently having 1-20 heteroatoms, or any combinations of any one or more of such moieties, wherein one or more methylene units of the group are optionally and independently replaced with C₁₋₆ alkylene, C₁₋₆ alkenylene, a bivalent C₁₋₆ heteroaliphatic group having 1-5 heteroatoms, —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or —[(—O—C(R′)₂—C(R′)₂—)n]—, wherein n is 1-20;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms; and
MOI is peptide having at least 90% identity with a sequence selected from:
(SEQ ID NO: 1)
DEDLEELERLYRKAEEVAKEAKDASRRGDDERAKEQMERAMRLFDQVFE
LAQELQEKQTDGNRQKATHLDKAVKEAADELYQRVR,
(SEQ ID NO: 2)
ELEEQVMHVLDQVSELAHELLHKLTGEELERAAYFNWWATEMMLELIKS
DDEREIREIEEEARRILEHLEELARK,
(SEQ ID NO: 3)
DKEWILQKIYEIMRLLDELGHAEASMRVSDLIYEFMKKGDERLLEEAER
LLEEVER,
(SEQ ID NO: 4)
DKEEILNKIYEIMRLLDELGNAEASMRVSDLILEFMKKGDERLLEEAER
LLEEVER,
(SEQ ID NO: 5)
SDDEDSVRYLLYMAELRYEQGNPEKAKKILEMAEFIAKRNNNEELERLV
REVKKRL,
(SEQ ID NO: 6)
NDDELHMLMTDLVYEALHFAKDEEIKKRVFQLFELADKAYKNNDRQKLE
KVVEELKELLERLLS,
(SEQ ID NO: 7)
QREKRLKQLEMLLEYAIERNDPYLMFDVAVEMLRLAEENNDERIIERAK
RILEEYE,
(SEQ ID NO: 8)
SLEELKEQVKELKKELSPEMRRLIEEALRFLEEGNPAMAMMVLSDLVYQ
LGDPRVIDLYMLVTKT,
(SEQ ID NO: 9)
DREQRLVRFLVRLASKFNLSPEQILQLFEVLEELLERGVSEEEIRKQLE
EVAKELG,
(SEQ ID NO: 10)
DDDIRYLIYMAKLRLEQGNPEEAEKVLEMARFLAERLGMEELLKEVREL
LRKIEELR,
and
(SEQ ID NO: 11)
PIIELLREAKEKNDEFAISDALYLVNELLQRTGDPRLEEVLYLIWRALK
EKDPRLLDRAIELFER.

18. The agent of claim 17, wherein the antibody binding moiety comprises or has the structure of
(i) DCAWHLGELVWCT (SEQ ID NO:35) or a salt form thereof, wherein the two C residues are linked by a —S—S—;
(ii) DCAWHLGELVWCT (SEQ ID NO:35) or a salt form thereof, wherein the N-terminus is capped with R—C(O)—; or
(iii) DCAWHLGELVWCT (SEQ ID NO:35) or a salt form thereof, wherein the N-terminus is capped with R—C(O)—, wherein R is methyl;
(iv) DCAWHLGELVWCT (SEQ ID NO:35) or a salt form thereof, wherein the antibody binding moiety is connected to the rest of a molecule through its C-terminus.

19. The agent of claim 17, wherein the antibody binding moiety comprises or has the structure selected from A-1 to A-50, or a salt form thereof:

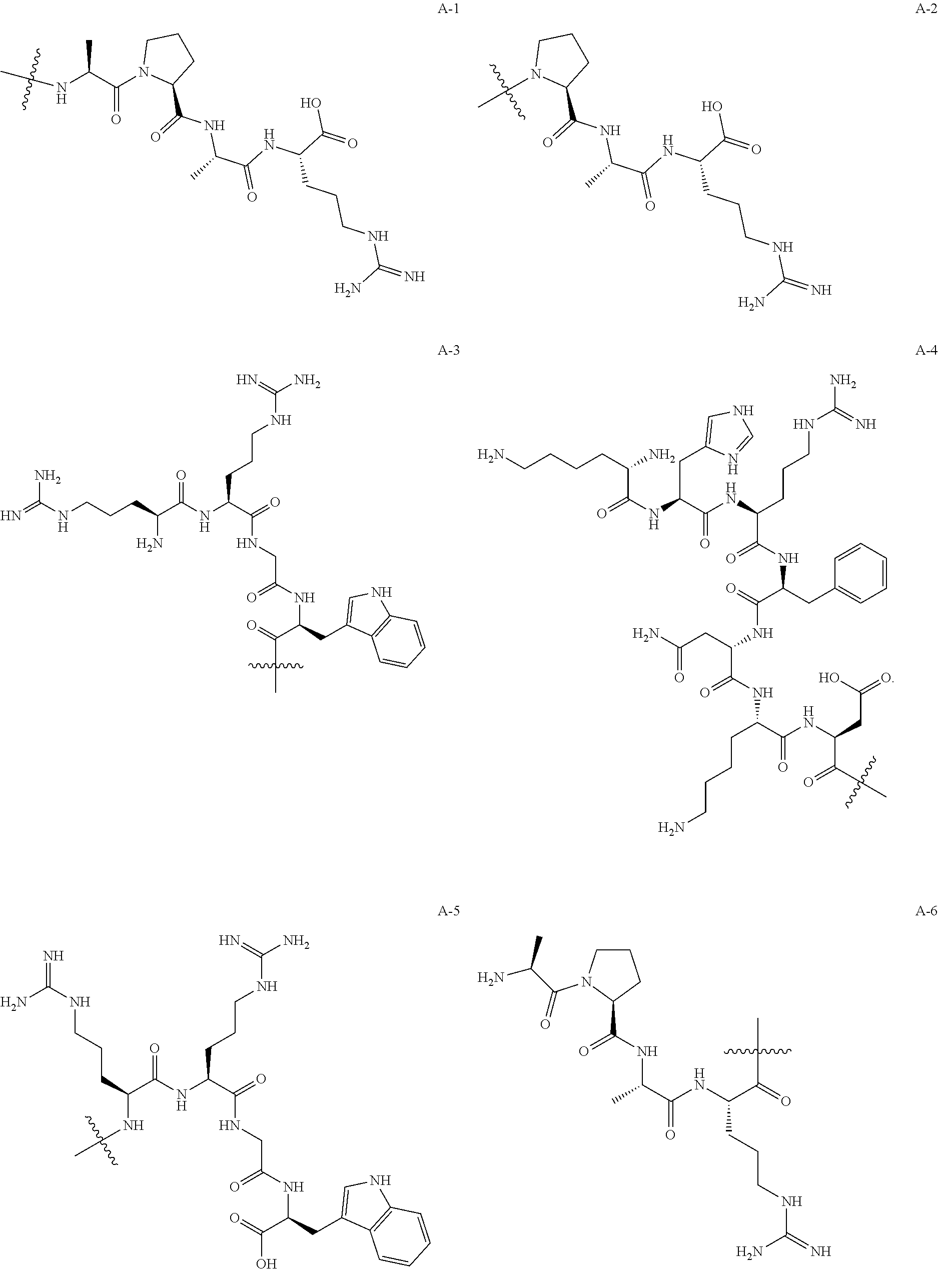

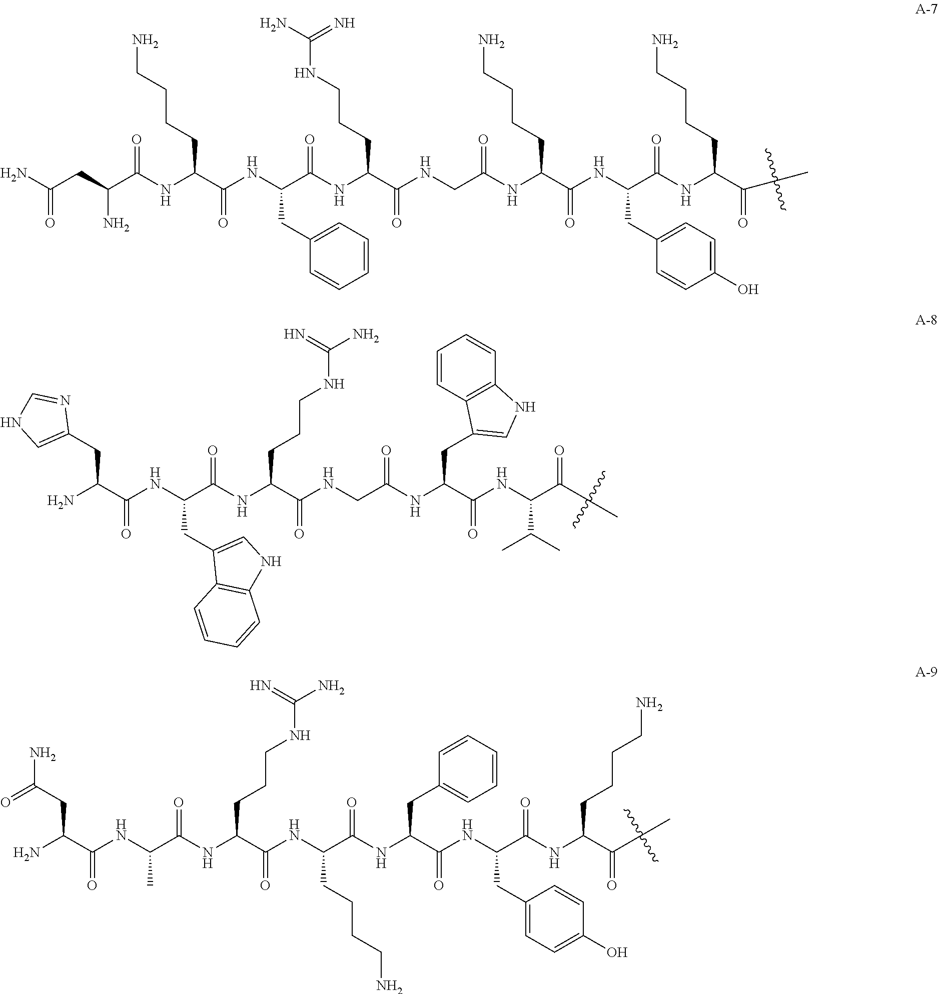 [SMILES: *C(=O)[C@H](CCCCN)NC(=O)[C@H](Cc1ccc(O)cc1)NC(=O)[C@H](CCCCN)NC(=O)CNC(=O)[C@H](CCCNC(=N)N)NC(=O)[C@H](Cc1ccccc1)NC(=O)[C@H](CCCCN)NC(=O)[C@@H](N)CC(N)=O ; *C(=O)[C@@H](NC(=O)[C@H](Cc1c[nH]c2ccccc12)NC(=O)CNC(=O)[C@H](CCCNC(=N)N)NC(=O)[C@H](Cc1c[nH]c2ccccc12)NC(=O)[C@@H](N)Cc1c[nH]cn1)C(C)C ; *C(=O)[C@H](CCCCN)NC(=O)[C@H](Cc1ccc(O)cc1)NC(=O)[C@H](Cc1ccccc1)NC(=O)[C@H](CCCCN)NC(=O)[C@H](CCCNC(=N)N)NC(=O)[C@H](C)NC(=O)[C@@H](N)CC(N)=O]

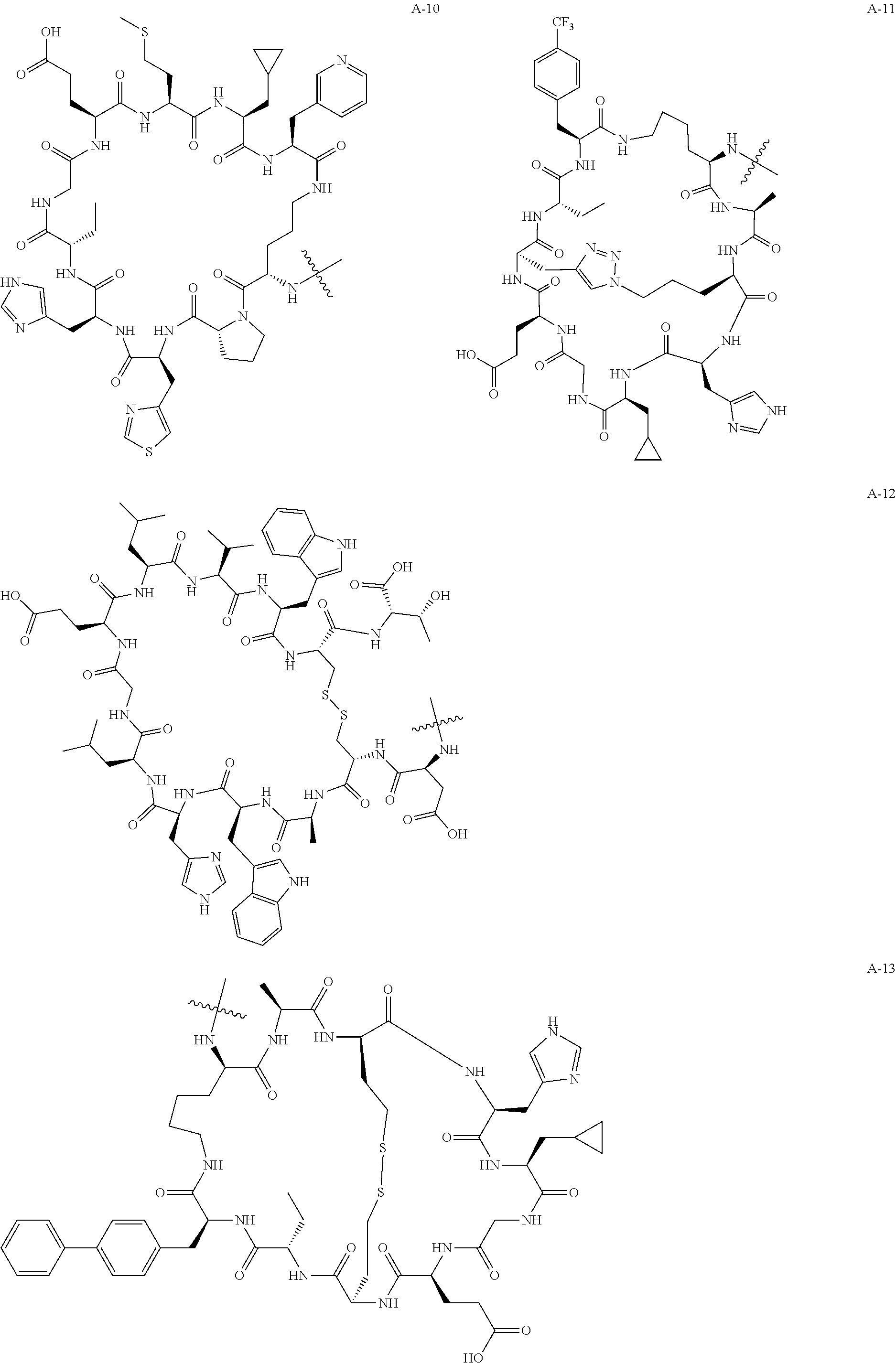

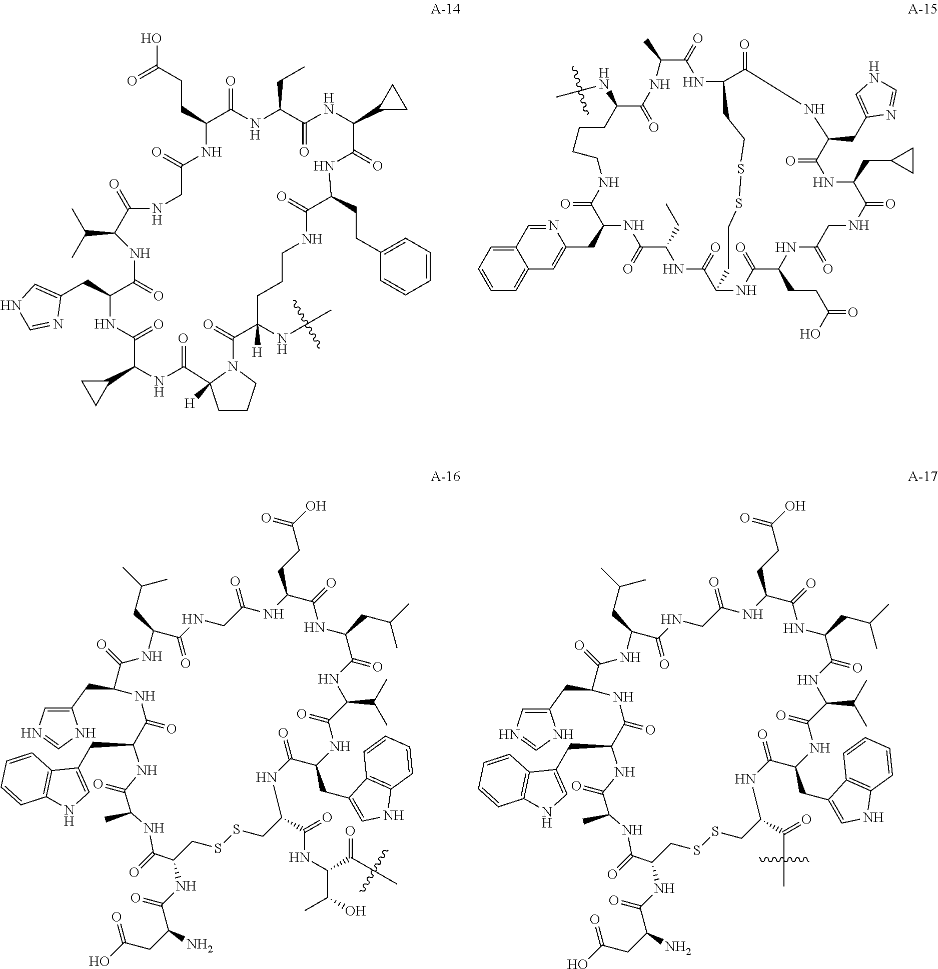

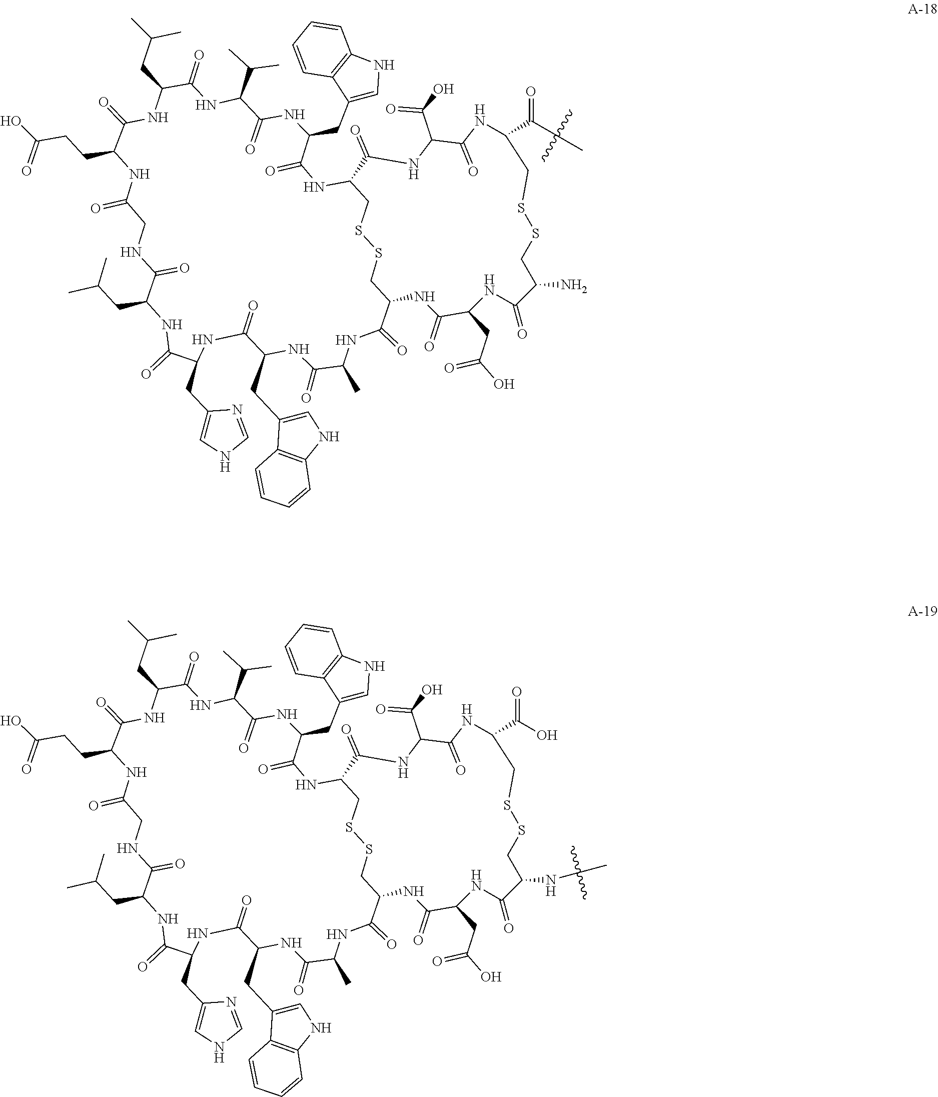

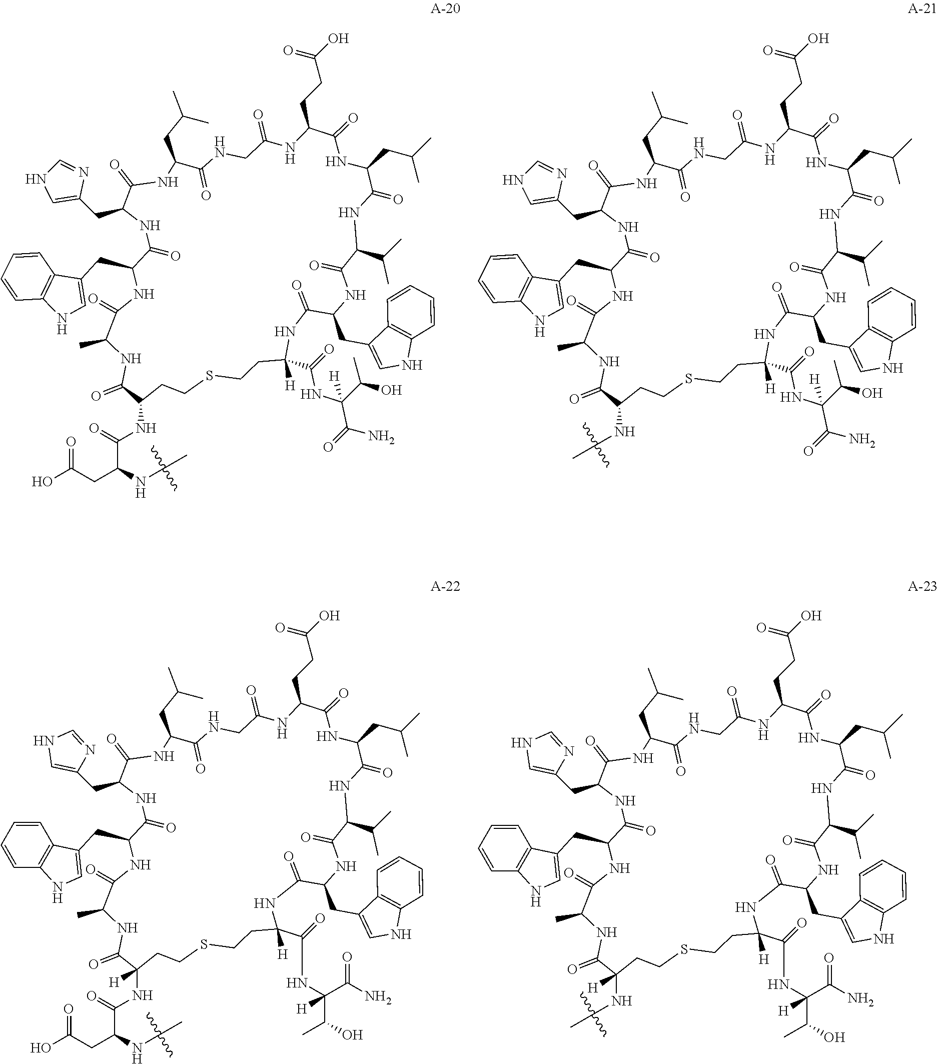

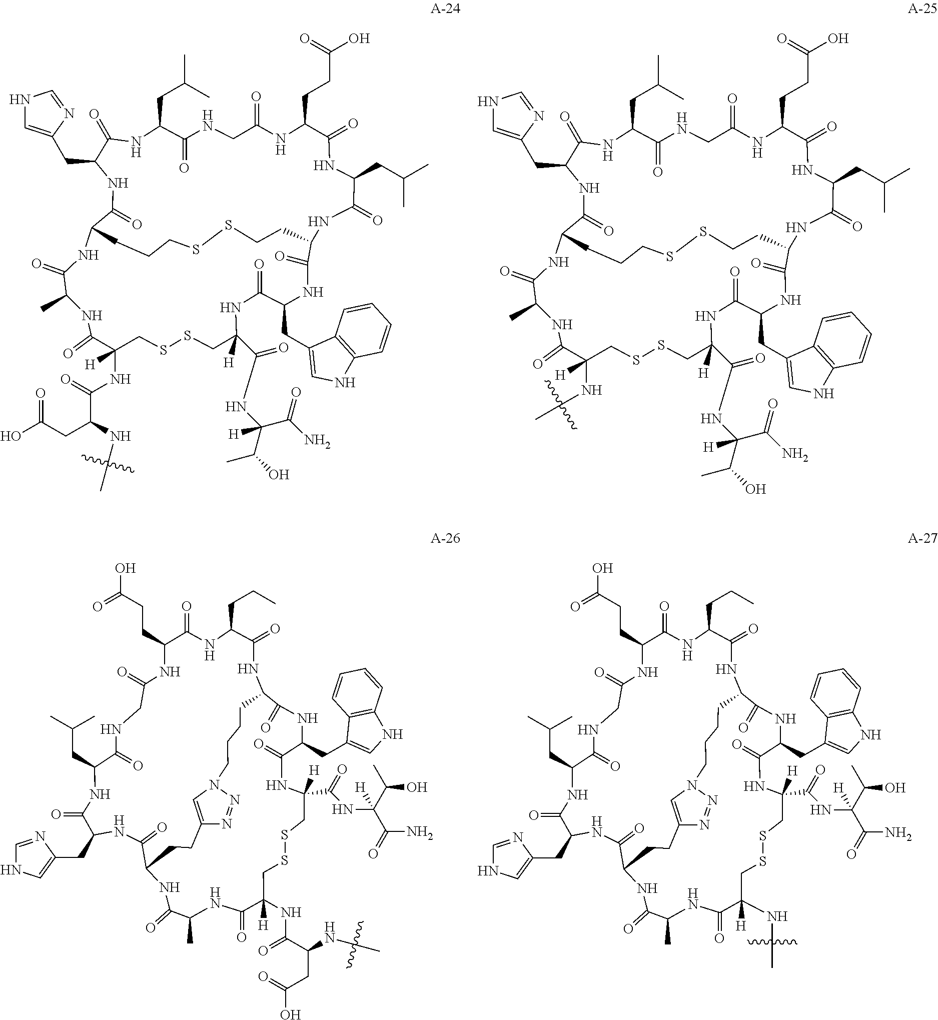

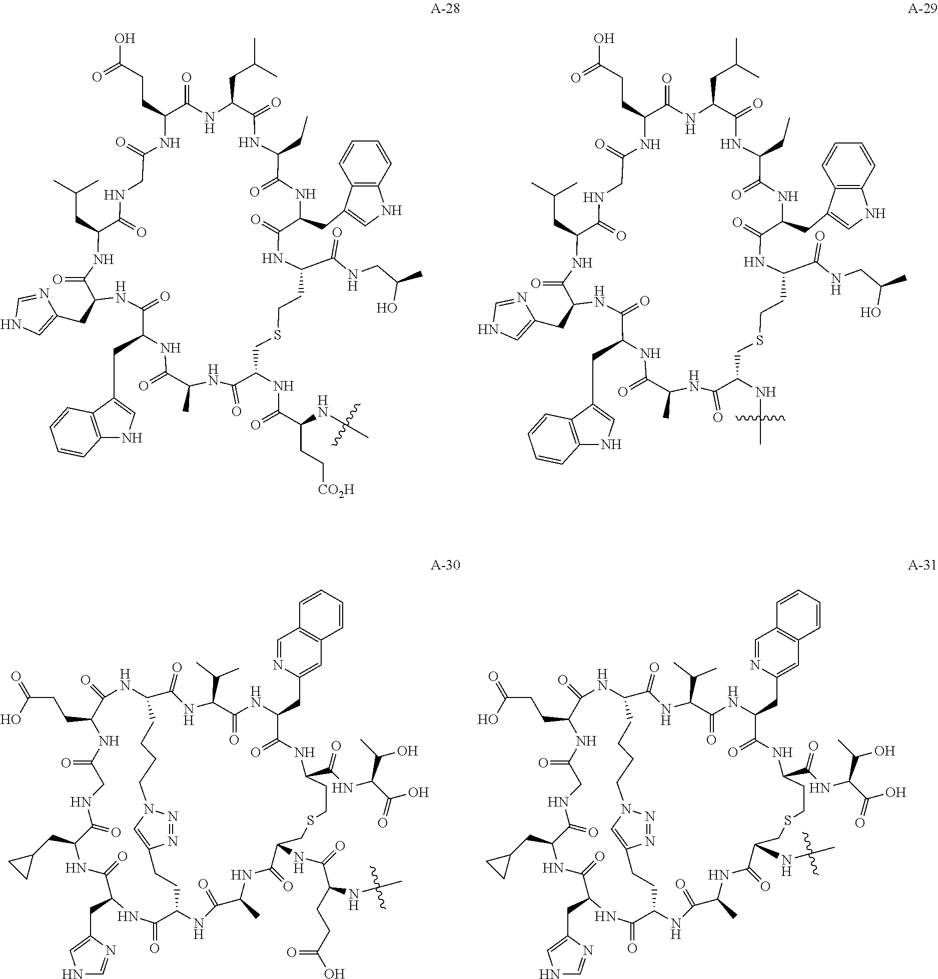

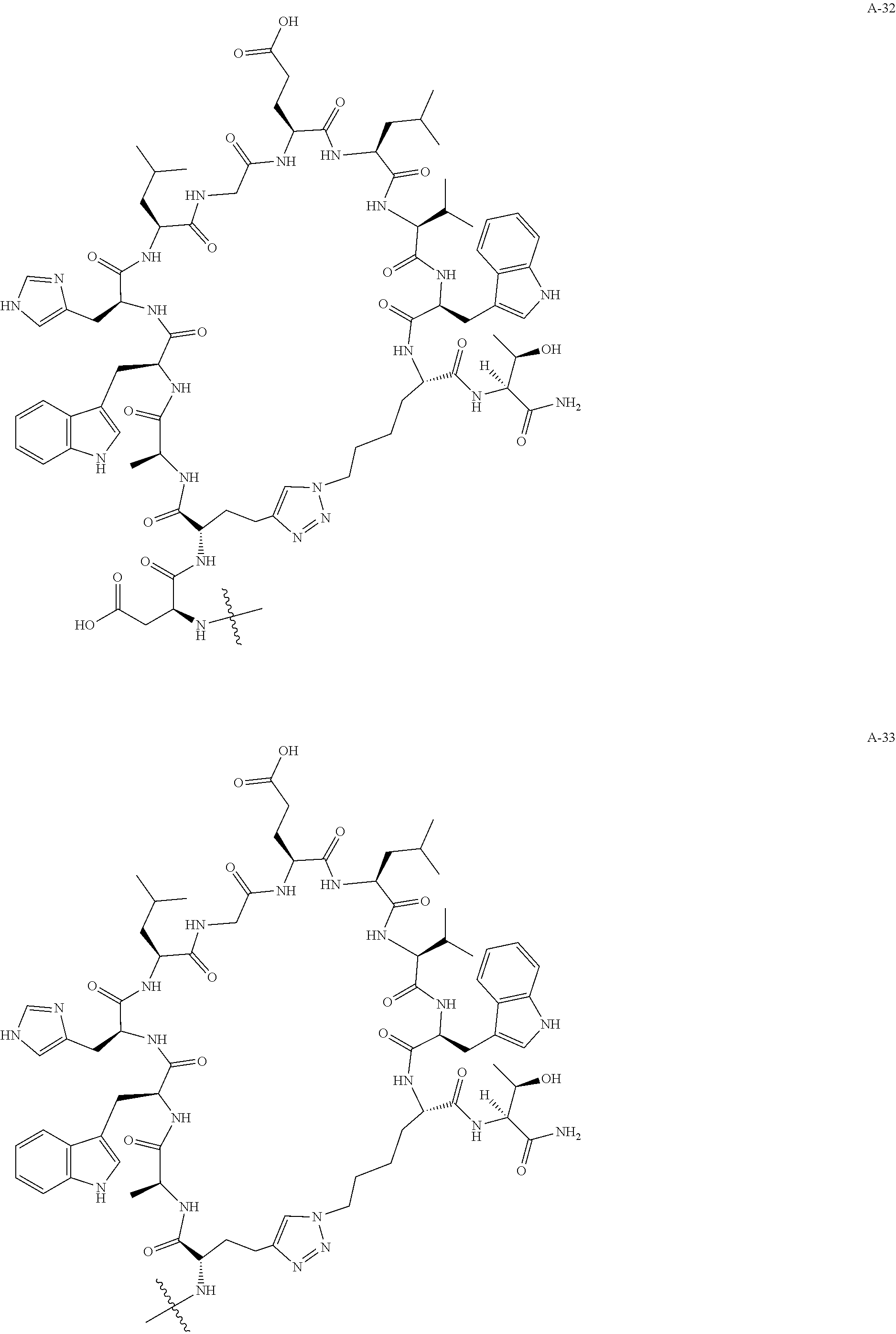

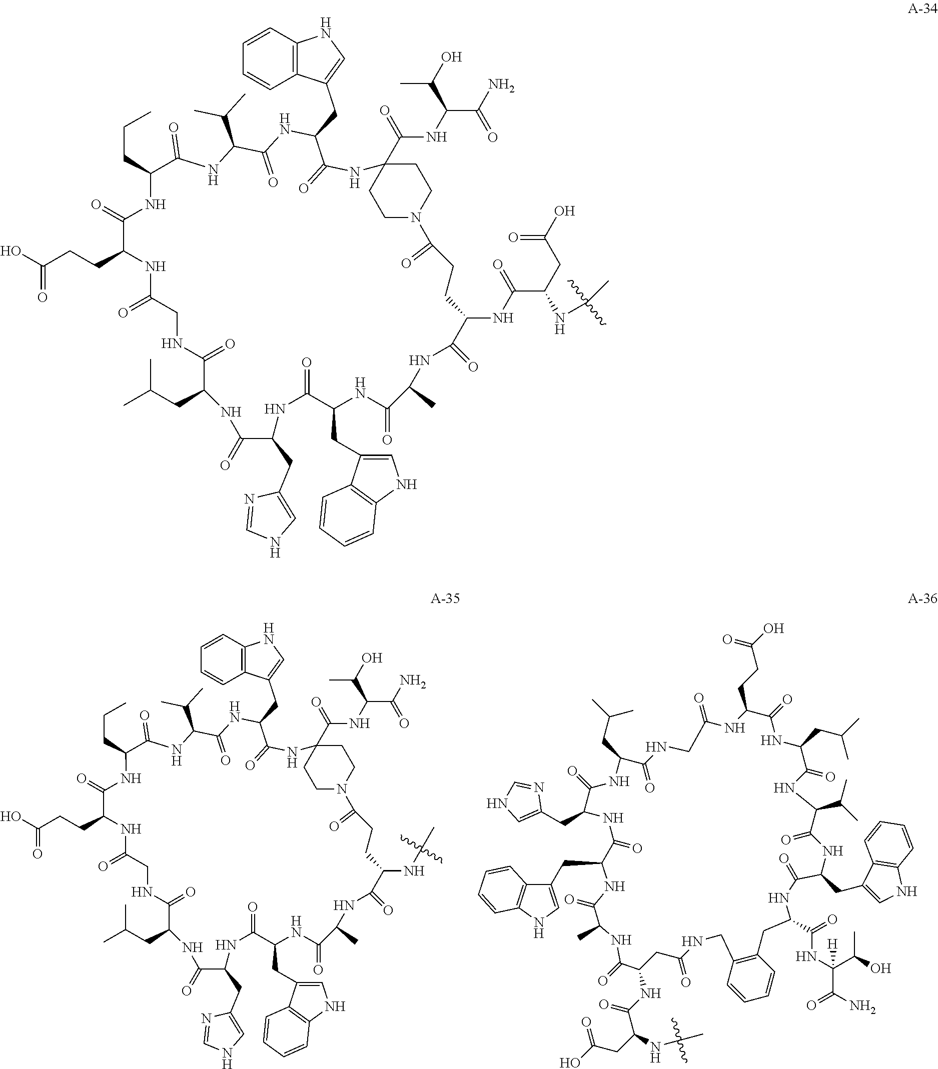

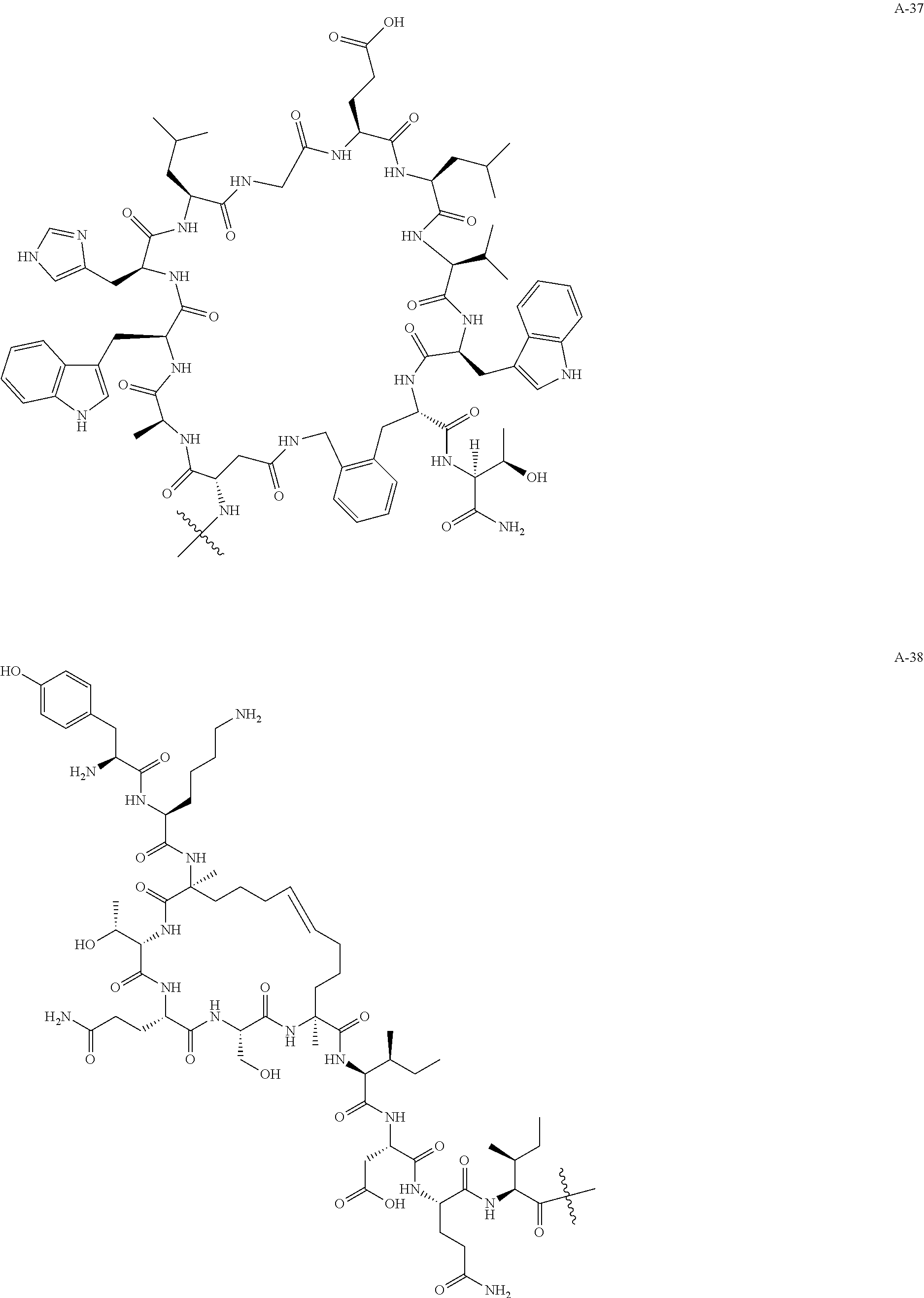

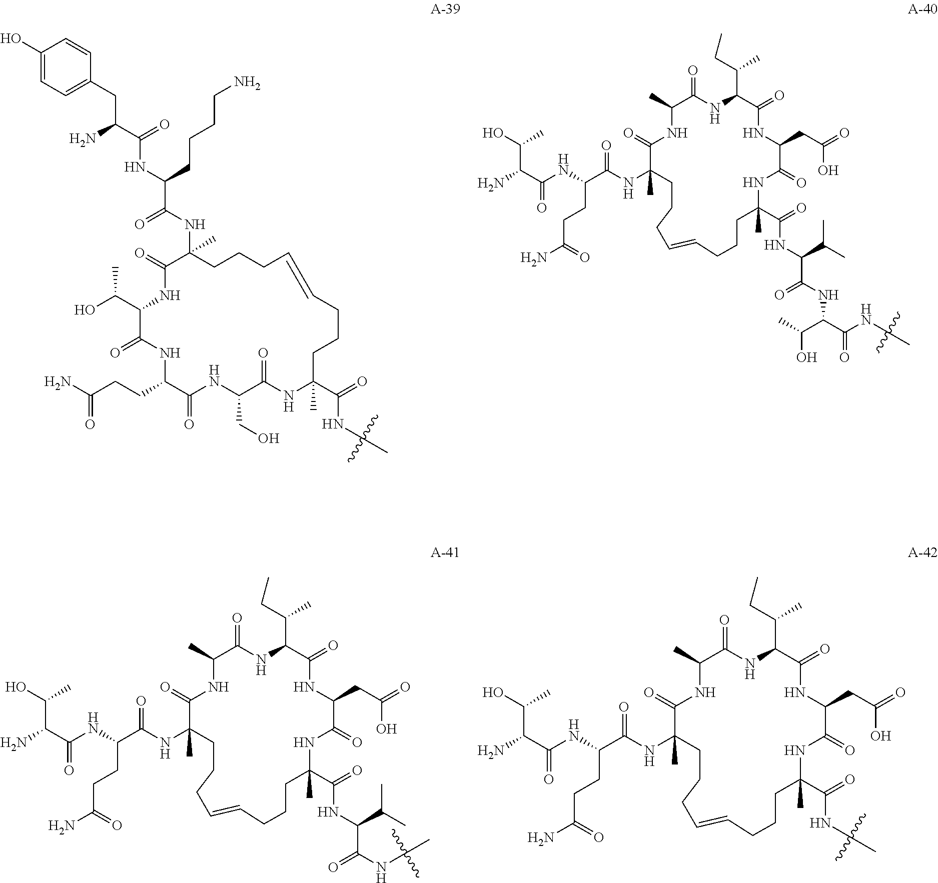

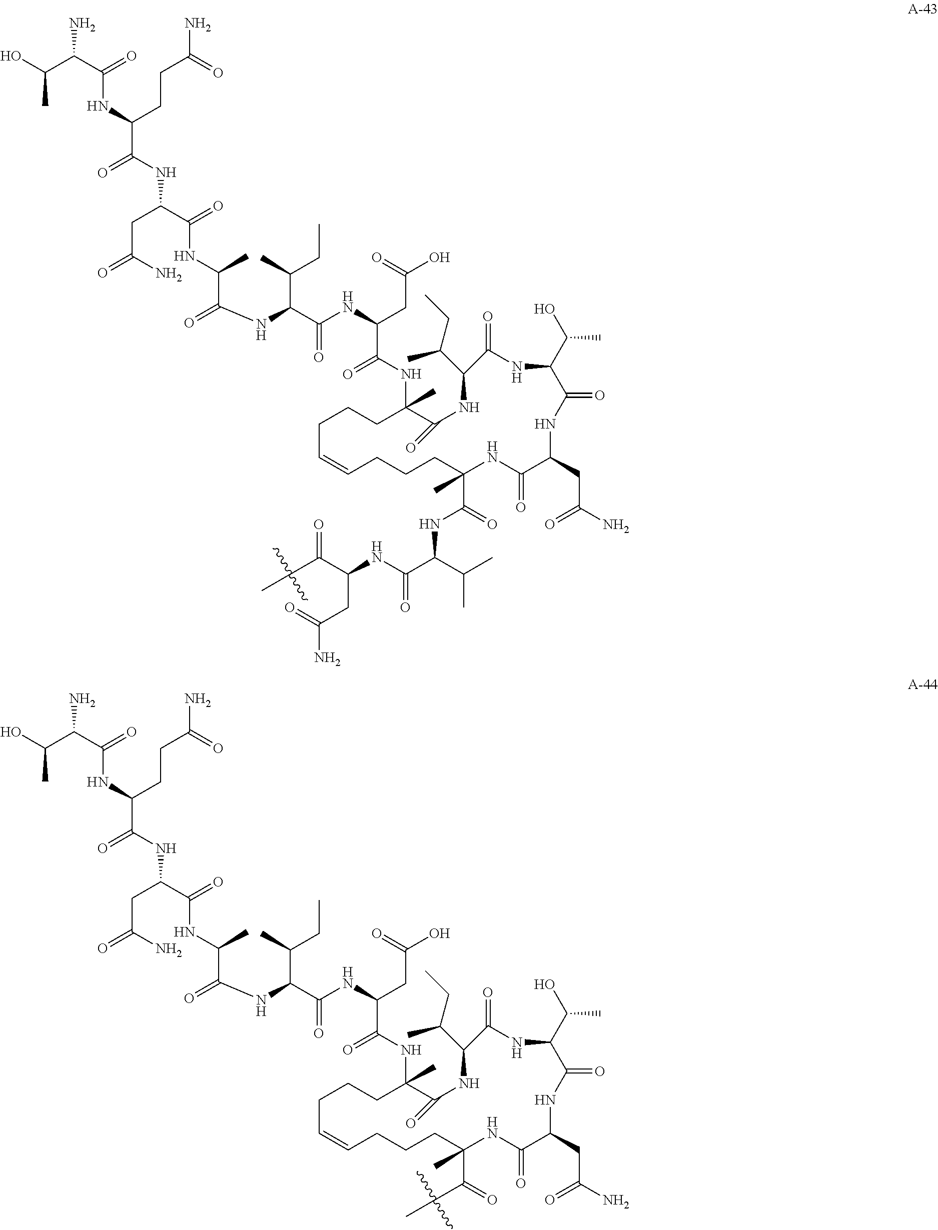

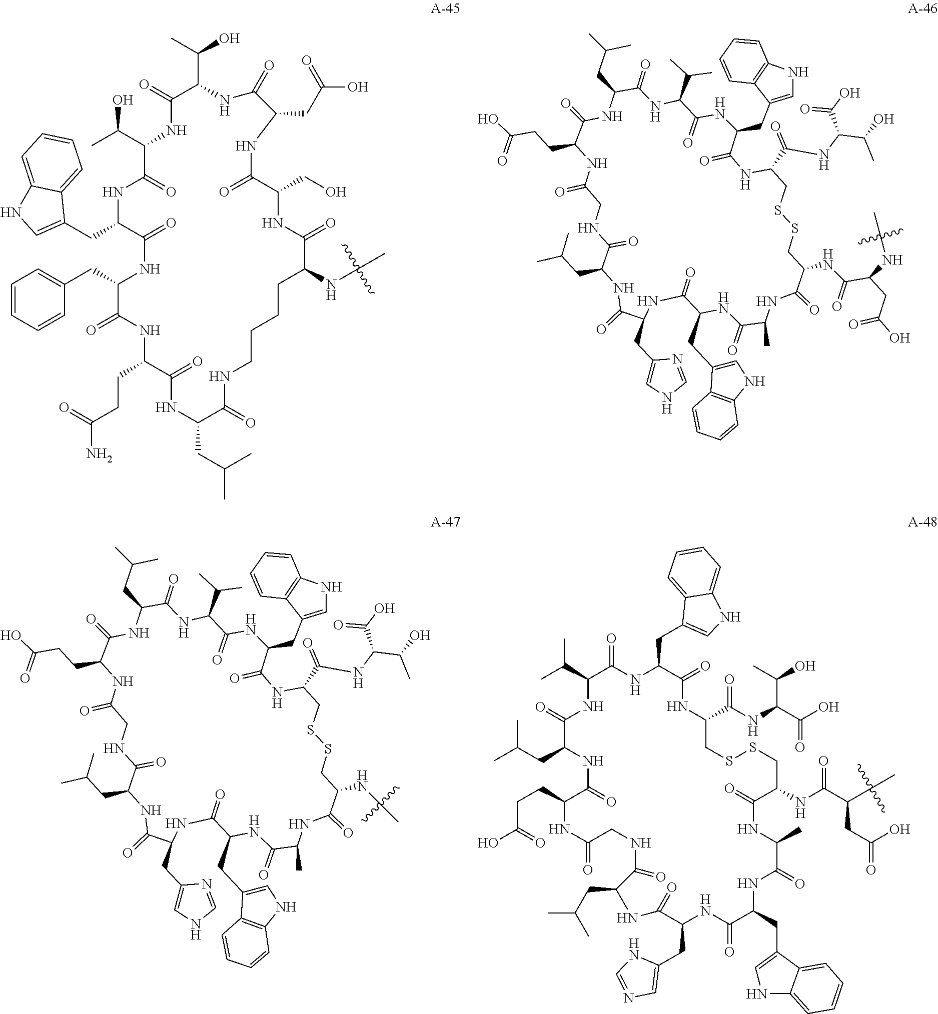

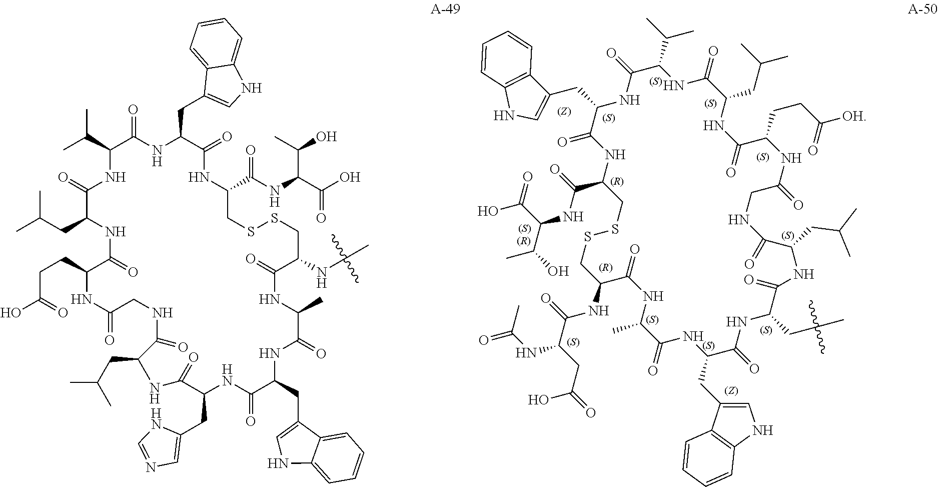

20. The agent of claim 1, wherein each L is independently a covalent bond, or a bivalent optionally substituted, linear or branched aliphatic group or heteroaliphatic group having 1-10 heteroatoms, wherein one or more methylene units of the group are optionally and independently replaced with —C≡C—, -Cy-, —C(R′)₂—, —O—, —S—, —S—S—, —N(R′)—, —C(O)—, —C(S)—, —C(NR′)—, —C(O)N(R′)—, —C(O)C(R′)₂N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, an amino acid residue, or —[(—O—C(R′)₂—C(R′)₂—)ₙ]—, wherein n is 1-20.

21. The agent of claim 17, wherein
(LG is R^{LG}-L^{LG}-, wherein R^{LG} is or comprises a target binding moiety and L^{LG} is L^{LG1}, L^{LG1}-L^{LG2}-, L^{LG1}-L^{LG2}-L^{LG3}-, or L^{LG1}-L^{LG2}-L^{LG3}-L^{LG4}-, where each L^{LG} is independently chosen from L.

22. The agent of claim 17, wherein RG is or comprises -L^{LG2}-L^{LG3}-L^{LG4}-L^{RG1}-L^{RG1}, -L^{LG2}-L^{LG3}-L^{LG4}-L^{RG1}-L^{RG2}-, -L^{LG3}-L^{LG4}-L^{RG1}-L^{RG2}-, L^{LG4}-L^{RG1}-L^{RG2}-, or -L^{RG1}-L^{RG2}-, where
L^{LG1} is a covalent bond, —(CH₂CH₂O)n-, or —(CH₂)n-O—(CH₂CH₂O)n-(CH₂)n-;
L^{LG2} is or comprises a covalent bond, —NR′—, —C(O)—, —NR′C(O)—, —(CH₂)n-OC(O)N(R′)—, —CH₂N(CH₂CH₂CH₂S(O)₂OH)—C(O)—, —C(O)—NHCH₂—, —C(O)O—CH₂—, or —NH—C(O)O—CH₂—; R′ is H or C₁-C₆alkyl;
L^{LG3} is optionally bonded to —C(O)— and L^{LG3} a covalent bond or a substituted phenyl ring, substituted with one or more substituents, and one or more substituents are independently an electron-withdrawing group;
L^{LG3} is 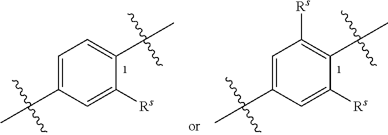
where R^{S} is F or NO₂;
L^{LG4} is a covalent bond, —O—, —NR′—;
L^{RG1} is a covalent bond, —S(O)₂— or —C(O)—;
L^{RG2} is or comprises —C(O)—, —C(O)O—, —C(O)N(R′)—, —S(O)—, —S(O)₂—, —P(O)(OR′)—, —P(O)(SR′)—, or —P(O)(N(R′)₂)—;
wherein each n is independently 1-10, and each —CH₂— is independently optionally substituted.

23-24. (canceled)

25. The agent of claim 19, wherein -L^{LG2}-L^{LG3}-L^{LG4}-L^{RG1}- is a structure selected from:

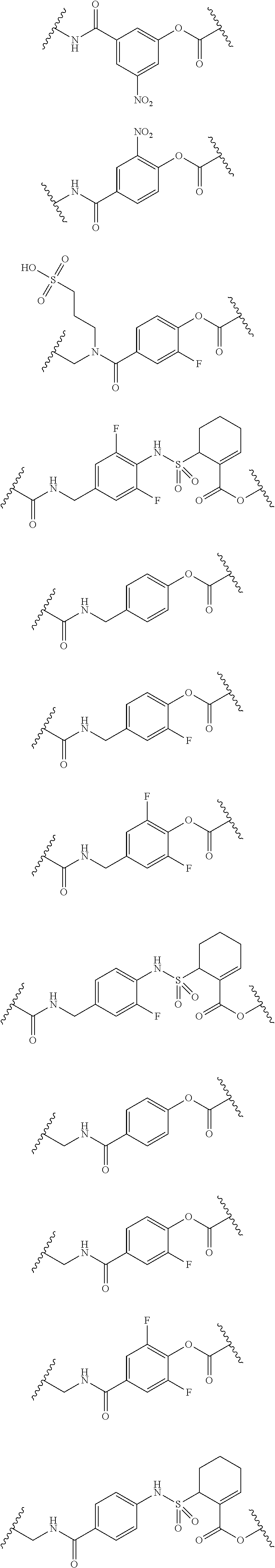

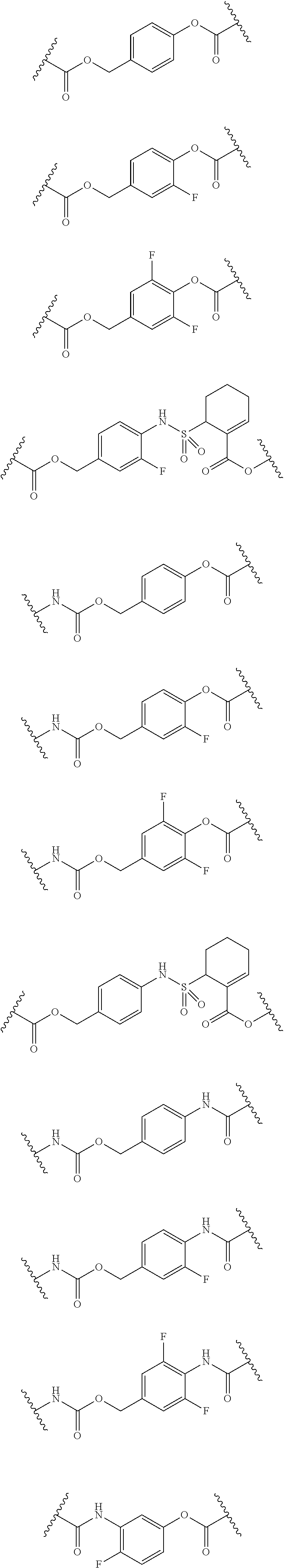

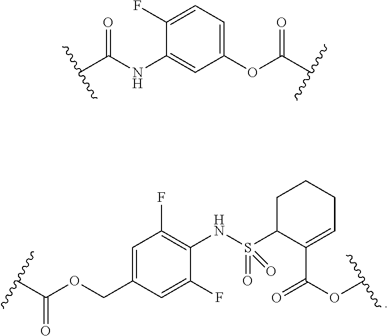 [SMILES: *C(=O)Nc1cc(OC(*)=O)ccc1F ; *OC(=O)C1=CCCCC1S(=O)(=O)Nc1c(F)cc(COC(*)=O)cc1F]

26. The agent of claim 1, wherein the target binding moiety binds to a SARS-CoV-2 virus particle, such as a protein of a SARS-CoV-2 virus, such as a spike protein of a SARS-Co-V-2 virus, such as spike protein or a fragment thereof of a SARS-CoV-2 virus expressed by an infected cell, or such as SARS-CoV-2 spike receptor binding domain.

27-30. (canceled)

31. A method of treating SARS-CoV-2 in a mammal comprising administering to the mammal a concentration of the agent of claim 1 sufficient to agent of any one of the provides long term immunity to SARS-CoV-2 to the mammal.

32. (canceled)

33. A composition comprising a plurality of agents, wherein each agent independently comprises:
an antibody moiety,
a target binding moiety, and
optionally a linker moiety linking an antibody binding moiety and a target binding moiety, wherein the agents of the plurality share the same antibody moiety, linker moiety, and target binding moiety;
wherein the target binding moiety is a peptide having at least 90% identify with a sequence selected from:
(SEQ ID NO: 1)
DEDLEELERLYRKAEEVAKEAKDASRRGDDERAKEQMERAMRLFDQVFE
LAQELQEKQTDGNRQKATHLDKAVKEAADELYQRVR,
(SEQ ID NO: 2)
ELEEQVMHVLDQVSELAHELLHKLTGEELERAAYFNWWATEMMLELIKS
DDEREIREIEEEARRILEHLEELARK,
(SEQ ID NO: 3)
DKEWILQKIYEIMRLLDELGHAEASMRVSDLIYEFMKKGDERLLEEAER
LLEEVER,
(SEQ ID NO: 4)
DKEEILNKIYEIMRLLDELGNAEASMRVSDLILEFMKKGDERLLEEAER
LLEEVER,
(SEQ ID NO: 5)
SDDEDSVRYLLYMAELRYEQGNPEKAKKILEMAEFIAKRNNNEELERLV
REVKKRL,
(SEQ ID NO: 6)
NDDELHMLMTDLVYEALHFAKDEEIKKRVFQLFELADKAYKNNDRQKLE
KVVEELKELLERLLS,
(SEQ ID NO: 7)
QREKRLKQLEMLLEYAIERNDPYLMFDVAVEMLRLAEENNDERIIERAK
RILEEYE,
(SEQ ID NO: 8)
SLEELKEQVKELKKELSPEMRRLIEEALRFLEEGNPAMAMMVLSDLVYQ
LGDPRVIDLYMLVTKT,
(SEQ ID NO: 9)
DREQRLVRFLVRLASKFNLSPEQILQLFEVLEELLERGVSEEEIRKQLE
EVAKELG,
(SEQ ID NO: 10)
DDDIRYLIYMAKLRLEQGNPEEAEKVLEMARFLAERLGMEELLKEVREL
LRKIEELR,
and
(SEQ ID NO: 11)
PIIELLREAKEKNDEFAISDALYLVNELLQRTGDPRLEEVLYLIWRALK
EKDPRLLDRAIELFER;
and
the antibody comprises IgG1, IgG2, IgG4, an Fc region, or a fragment of any of the foregoing.

34-36. (canceled)

37. The composition of claim 33, wherein one or more agents of the plurality can each independently interact hFcγRIIIA.

38-41. (canceled)

42. The agent of claim 1, where the agent is selected from 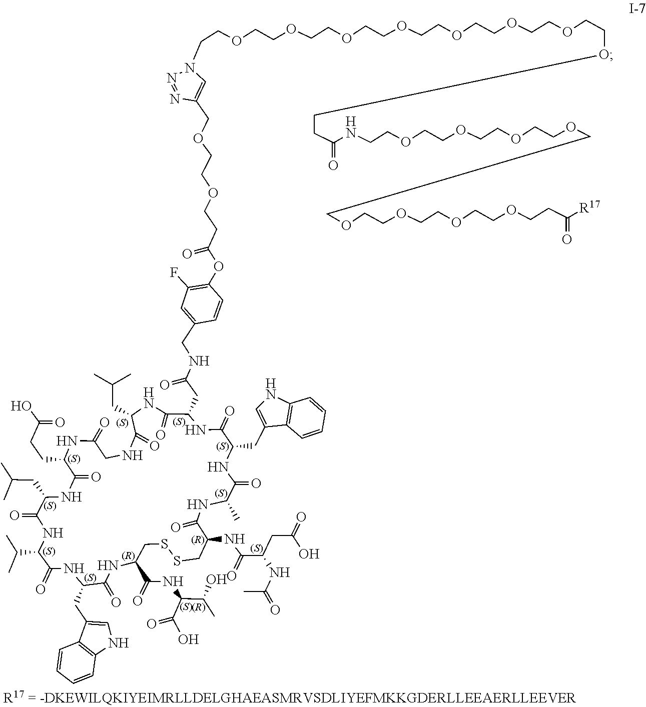 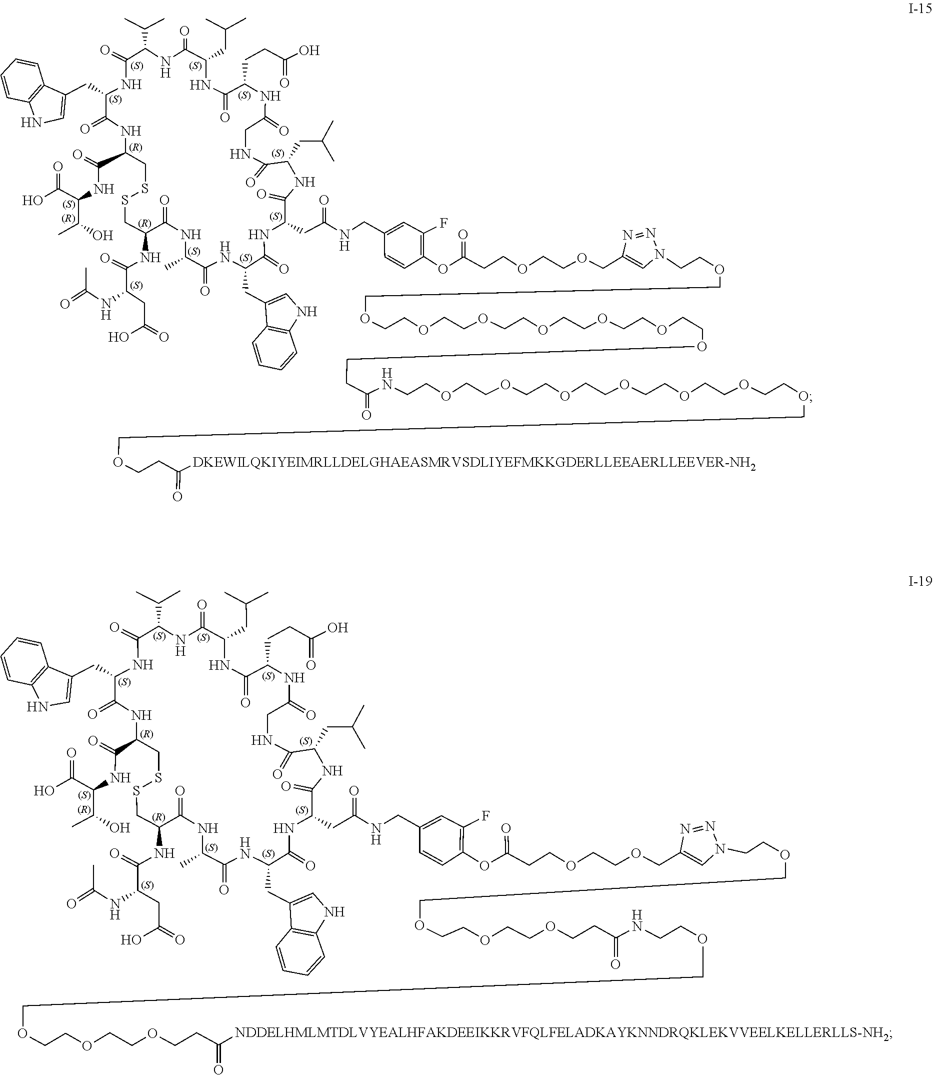 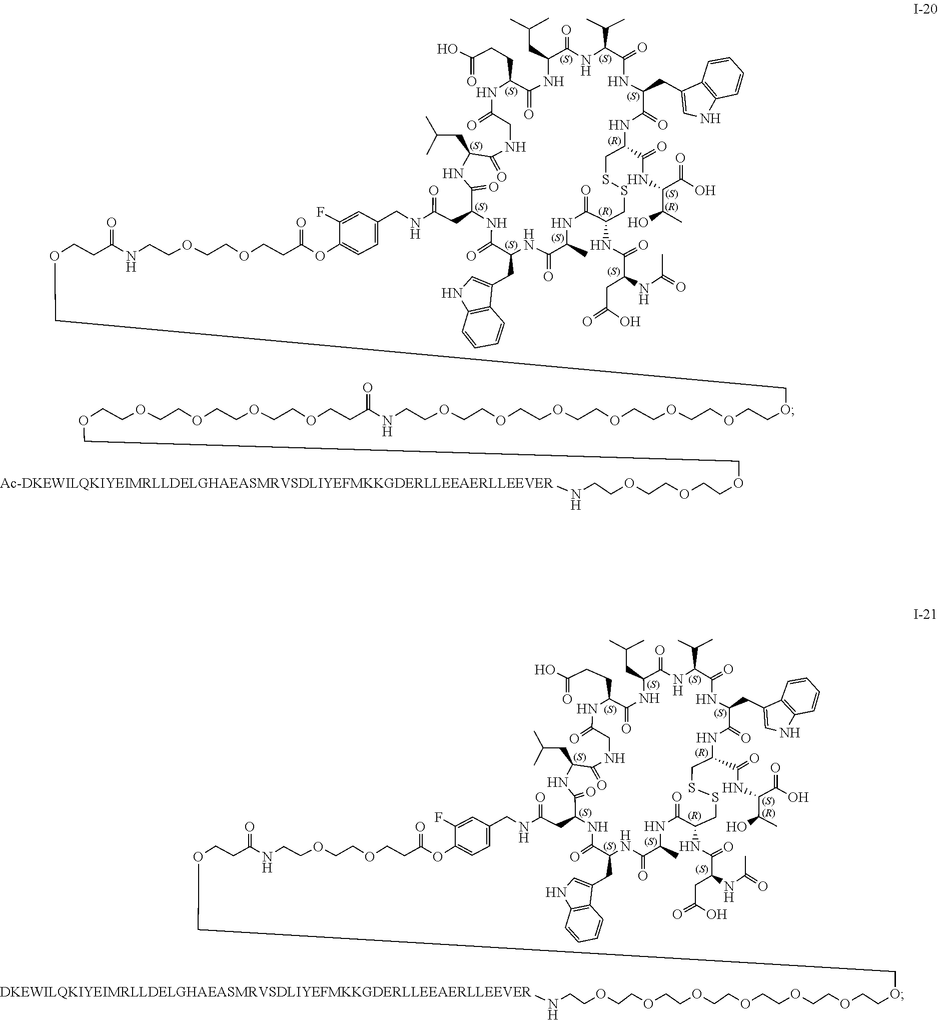 [chemical structure]
